(19) United States
(12) Patent Application Publication
(10) Pub. No.: US 2025/0276066 A1
(43) Pub. Date: Sep. 4, 2025

(54) METHODS, COMPOSITIONS AND COMPOUNDS FOR TREATING AGE-RELATED DISEASES AND CONDITIONS

(71) Applicant: GERO PTE. LTD., Singapore (SG)

(72) Inventors: Andrei Evgenevich TARKHOV; Konstantin Aleksandrovich AVKHACHEV; Olga Andreevna BURMISTOROVA; Maksim Nikolaevich KHOLIN; Petr Olegovich FEDICHEV

(21) Appl. No.: 18/264,805

(22) PCT Filed: Feb. 9, 2022
(86) PCT No.: PCT/RU2022/050044

§ 371 Date: Aug. 9, 2023

(30) Foreign Application Priority Data
Oct. 2, 2021 (RU) 2021103259

(51) Int. Cl. A61K 45/06; A61K 31/365; A61K 31/4045; A61K 31/4406; A61K 31/454; A61K 31/475; A61K 31/5377; A61P 43/00

(52) U.S. Cl. CPC A61K 45/06; A61K 31/365; A61K 31/4045; A61K 31/4406; A61K 31/454; A61K 31/475; A61K 31/5377; A61P 43/00

(57) ABSTRACT

Disease target identification is among the most difficult problems in biomedicine. The sheer size of genetic signal (number of observed features per genome) poses a nearly insurmountable challenge of the required number of samples for association studies. Here, we applied a state-of-the-art deep neural network for feature selection (dimensionality reduction) and non-biological variance (batch) removal in human cells. By integrating genome-wide shRNA targeting, exome-wide ultra-rare mutations, and large cohort survival data, we obtained gene expression signatures associated with extended lifespan. In turn, these signatures allowed us to identify small molecules based on their association with longevity and prioritize known and novel targets of anti-aging therapeutics.

### FIELD

We disclose the anti-aging, senolytic and other therapeutic effects of several compounds and their analogs and combinations described herein as well as related methods.

### BACKGROUND

The development of methods, medications for increasing health span and/or treating aging or age-related diseases would benefit many.

New targets, compounds, compositions for treating aging or age-related conditions are desired.

### SUMMARY

The present inventors identified therapeutic effects of several compounds and their analogs and combinations described herein as well as related methods.

Accordingly, the present application includes new anti-aging and other therapeutic uses disclosed herein of modulators of target selected from the group of targets consisting of: HTR2A, SLC6A3, SLC6A2, SCN2A, BMX, ADRA1B, FGFR1, EGFR, HRH1, HSP90AA1, HTR2C, CNR1, HDAC10, ERBB2, MAP3K19, BRD2, BRD4, PIK3CD, NR3C1, HTR2B, ERBB3, BLK, KDR, EPHA8, ADRA1A, EPHA6, HDAC9, PIK3CA, FLT3, ERBB4, DRD5, PIK3R1, HTR7, HDAC1, SCN3A, EBP, BRD3, DRD3, HDAC4, HDAC2, STK24, HDAC8, HDAC6, SCN1A, FLT1, IGF1R, HDAC11, MAP4K5, KCNH2, HDAC7, STK10, HMGCR, SLC6A4, ABL1, PDGFRB, HDAC3, LCK, ADRA1D, HTR6, DRD2, KIT, HDAC5, DDR1, ABL2.

In some embodiments, the term “Target” means any one of the proteins selected from the group consisting of: HTR2A, SLC6A3, SLC6A2, SCN2A, BMX, ADRA1B, FGFR1, EGFR, HRH1, HSP90AA1, HTR2C, CNR1, HDAC10, ERBB2, MAP3K19, BRD2, BRD4, PIK3CD, NR3C1, HTR2B, ERBB3, BLK, KDR, EPHA8, ADRA1A, EPHA6, HDAC9, PIK3CA, FLT3, ERBB4, DRD5, PIK3R1, HTR7, HDAC1, SCN3A, EBP, BRD3, DRD3, HDAC4, HDAC2, STK24, HDAC8, HDAC6, SCN1A, FLT1, IGF1R, HDAC11, MAP4K5, KCNH2, HDAC7, STK10, HMGCR, SLC6A4, ABL1, PDGFRB, HDAC3, LCK, ADRA1D, HTR6, DRD2, KIT, HDAC5, DDR1, ABL2.

In some embodiments, the term “Targets” means all of the proteins selected from the group consisting of: HTR2A, SLC6A3, SLC6A2, SCN2A, BMX, ADRA1B, FGFR1, EGFR, HRH1, HSP90AA1, HTR2C, CNR1, HDAC10, ERBB2, MAP3K19, BRD2, BRD4, PIK3CD, NR3C1, HTR2B, ERBB3, BLK, KDR, EPHA8, ADRA1A, EPHA6, HDAC9, PIK3CA, FLT3, ERBB4, DRD5, PIK3R1, HTR7, HDAC1, SCN3A, EBP, BRD3, DRD3, HDAC4, HDAC2, STK24, HDAC8, HDAC6, SCN1A, FLT1, IGF1R, HDAC11, MAP4K5, KCNH2, HDAC7, STK10, HMGCR, SLC6A4, ABL1, PDGFRB, HDAC3, LCK, ADRA1D, HTR6, DRD2, KIT, HDAC5, DDR1, ABL2.

In some embodiments, the term “Targets” means any combination of the proteins selected from the group consisting of: HTR2A, SLC6A3, SLC6A2, SCN2A, BMX, ADRA1B, FGFR1, EGFR, HRH1, HSP90AA1, HTR2C, CNR1, HDAC10, ERBB2, MAP3K19, BRD2, BRD4, PIK3CD, NR3C1, HTR2B, ERBB3, BLK, KDR, EPHA8, ADRA1A, EPHA6, HDAC9, PIK3CA, FLT3, ERBB4, DRD5, PIK3R1, HTR7, HDAC1, SCN3A, EBP, BRD3, DRD3, HDAC4, HDAC2, STK24, HDAC8, HDAC6, SCN1A, FLT1, IGF1R, HDAC11, MAP4K5, KCNH2, HDAC7, STK10, HMGCR, SLC6A4, ABL1, PDGFRB, HDAC3, LCK, ADRA1D, HTR6, DRD2, KIT, HDAC5, DDR1, ABL2.

In some embodiments, modulators of any one of the Targets are inhibitors. In some embodiments, modulators of Targets are small molecules. In some embodiments, modulators of any one of the Targets are small molecule inhibitors. In some embodiments such modulators of any one of the Targets are selected from Table Table 1 ab and Table 1c or their pharmaceutically acceptable salt or any combination of it or compounds or, optionally, having the similar SAR characteristics salts and/or solvates of any one of inhibitors of any of the Targets.

In another embodiment, the anti-aging compound binds and/or inhibits at least one of the Targets. In some embodiments, this invention is a method of anti-aging treatment, comprising administering of inhibitor of Target in therapeutically effective amount.

The application also provides an anti-aging therapy, comprising a compound selected from the group of all compounds of Table 1 ab and Table 1c.

In some embodiments, the application provides an anti-aging therapy, comprising an inhibitor of the protein, wherein protein is selected from the group consisting of: CDK7, CHEK2, CAPN1, CTSB, HSP90AB1, HSP90AA1, HSPA5, NFKB1, NPC1, PABPC1, ABCB11, ABCB1, PLK1, PLK3, ADRA2A, ADRA2C, ADRA2B, TUBB6, SENP8, SENP6, SENP7, MMP14, MMP13, MMP8, MMP3, MMP1, MAPKAPK2, CTSL, CTSK, CTSS, ATXN2, TBXAS1, CREBBP, PDGFRA, FLT4, FLT1, KDR, PDGFRB, FLT3, KIT, CSF1R, IKBKB, IKBKE, PGGT1B, FAAH, GFER, MAPK14, MAPK12, PRKAA1, ABCG2, ADAM17, BACE2, BACE1, TUBB6, CA7, CA2, CA1, CA5A, CA12, CA9, CA4, CTSB, FEN1, HDAC1, HDAC2, HDAC3, CBX1, SLC6A4, SLC6A2, SLC6A3, PPARD, PPARG, PPARA, PSEN2, HTR7, ADRA1B, SMN2, FYN, SRC, LCK, LYN, TARDBP, CLK2, CLK4, USP2, GBA, RECQL, BLM, WRN, CDK2, PRKAG3, TFPI, LTA4H, DPP7, BACE2, BACE1, CAPN1, CTSB, IGF1R, INSR, EYA2, HSPA5, NFKB1, TCF7L2, TARDBP, BRD4, ABCB1, ADRA1A, ADRA1B, ADRA1D, BRD2, BRD3, BRD4, DRD2, DRD3, DRD4, DRD5, EBP, EGFR, EPHA6, EPHA8, ERBB2, HDAC1, HDAC10, HDAC11, HDAC2, HDAC3, HDAC4, HDAC5, HDAC6, HDAC7, HDAC8, HDAC9, HRH1, HSP90AA1, HTR2A, HTR2B, HTR2C, HTR5B, HTR6, HTR7, KCNH2, MAP4K5, PIK3C2A, PIK3C2B, PIK3CA, PIK3CB, PIK3CD, PIK3CG, PIK3R1, PRKDC, SLC6A2, SLC6A3, SLC6A4.

In some embodiments, the application provides an anti-aging therapy, comprising an inhibitor of the gene, wherein gene is selected from the group consisting of: CDK7, CHEK2, CAPN1, CTSB, HSP90AB1, HSP90AA1, HSPA5, NFKB1, NPC1, PABPC1, ABCB11, ABCB1, PLK1, PLK3, ADRA2A, ADRA2C, ADRA2B, TUBB6, SENP8, SENP6, SENP7, MMP14, MMP13, MMP8, MMP3, MMP1, MAPKAPK2, CTSL, CTSK, CTSS, ATXN2, TBXAS1, CREBBP, PDGFRA, FLT4, FLT1, KDR, PDGFRB, FLT3, KIT, CSF1R, IKBKB, IKBKE PGGT1B, FAAH, GFER, MAPK14, MAPK12, PRKAA1, ABCG2, ADAM17, BACE2, BACE1, TUBB6, CA7, CA2, CA1, CA5A, CA12, CA9, CA4, CTSB, FEN1, HDAC1, HDAC2, HDAC3, CBX1, SLC6A4, SLC6A2, SLC6A3, PPARD, PPARG, PPARA, PSEN2, HTR7, ADRA1B, SMN2, FYN, SRC, LCK, LYN, TARDBP, CLK2, CLK4, USP2, GBA, RECQL, BLM, WRN, CDK2, PRKAG3, TFPI, LTA4H, DPP7, BACE2, BACE1, CAPN1, CTSB, IGF1R, INSR, EYA2, HSPA5, NFKB1, TCF7L2, TARDBP, BRD4, ABCB1, ADRA1A, ADRA1B, ADRA1D, BRD2, BRD3, BRD4, DRD2, DRD3, DRD4, DRD5, EBP, EGFR, EPHA6, EPHA8, ERBB2, HDAC1, HDAC10, HDAC11, HDAC2, HDAC3, HDAC4, HDAC5, HDAC6, HDAC7, HDAC8, HDAC9, HRH1, HSP90AA1, HTR2A, HTR2B, HTR2C, HTR5B, HTR6, HTR7, KCNH2, MAP4K5, PIK3C2A, PIK3C2B, PIK3CA, PIK3CB, PIK3CD, PIK3CG, PIK3R1, PRKDC, SLC6A2, SLC6A3, SLC6A4.

In some embodiments, the application provides an anti-aging therapy, comprising a compound selected from the group consisting of: CYCLOSPORINE, TARIQUIDAR, MARIMASTAT, PRINOMASTAT, APRATASTAT, YOHIMBINE, QUETIAPINE, DOXEPIN, MIANSERIN, ERGOTAMINE, PIPAMAZINE, PHENTOLAMINE, LISURIDE, TAMSULOSIN, DEXMEDETOMIDINE, INDORAMIN, RISPERIDONE, SERTINDOLE, XYLOMETAZOLINE, NAPHAZOLINE, TETRAHYDROZOLINE, DESIPRAMINE, ALFUZOSIN, SILODOSIN, TERAZOSIN, OLANZAPINE, ZOTEPINE, DROPERIDOL, THIORIDAZINE, CHLORPROMAZINE, FLUPHENAZINE, CARVEDILOL, OXYMETAZOLINE, HALOPERIDOL, PHENOXYBENZAMINE, NAFTOPIDIL, DOXAZOSIN, BROMOCRIPTINE, CLOZAPINE, PRAZOSIN, DIHYDROERGOTAMINE, CLOMIPRAMINE, AMITRIPTYLINE, IMIPRAMINE, NORTRIPTYLINE, ASTEMIZOLE, PROMAZINE, ZIPRASIDONE, PHENYLEPHRINE, CISAPRIDE, DAPIPRAZOLE, ILOPERIDONE, MORPHINE, ZOLMITRIPTAN, LOFEXIDINE, FIPAMEZOLE, FLUOXETINE, ATIPAMEZOLE, APRACLONIDINE, LEVONORDEFRIN, IDAZOXAN, BRIMONIDINE, GUANABENZ EPINEPHRINE, NOREPINEPHRINE, CLONIDINE, CLONIDINE, METERGOLINE, PROCHLORPERAZINE, LY-2811376, LANABECESTAT, AZD-3839, VERUBECESTAT, BIRABRESIB, MIVEBRESIB, VORINOSTAT, ALOBRESIB, AZD-5153, PANOBINOSTAT, ACETAZOLAMIDE, BRINZOLAMIDE, MAFENIDE, INDISULAM, INDAPAMIDE, DTP-348, TOPIRAMATE, LEVOSULPIRIDE, DORZOLAMIDE, SULPIRIDE, CHLORTHALIDONE, METHAZOLAMIDE, NILOTINIB, ETHOXZOLAMIDE, ZONISAMIDE, DICHLORPHENAMIDE, SULTHIAME, METOLAZONE, TRICHLORMETHIAZIDE, SACCHARIN, LUTEOLIN, ISOQUERCETIN, DAIDZEIN, FAMOTIDINE, AZD-5438, TRILACICLIB, RGB-286638, ZOTIRACICLIB, LEROCICLIB, PHA-793887, PALBOCICLIB, HEXAMETHYL PARAROSANILINE, RG-547, DINACICLIB, FORETINIB, AST-487, AT-7519, AZD-7762, UCN-01, SUNITINIB, SILMITASERTIB, LESTAURTINIB, EDICOTINIB, ILORASERTIB, PEXIDARTINIB, TAK-593, PAZOPANIB, TANDUTINIB, DOVITINIB, CEP-32496, QUIZARTINIB, LINIFANIB, MOTESANIB, MASITINIB, DASATINIB, IMATINIB, SU-014813, RELACATIB, BALICATIB, ODANACATIB, ROTIGOTINE, CLEBOPRIDE, RACLOPRIDE, SUMANIROLE, PRAMIPEXOLE, PF-00217830, PALIPERIDONE, BENPERIDOL, BREXPIPRAZOLE, BIFEPRUNOX, HALOPERIDOL DECANOATE, ARMODAFINIL, MODAFINIL, MESORIDAZINE, ASENAPINE, BLONANSERIN, CLOTHIAPINE, FALLYPRIDE, ROPINIROLE, SARIZOTAN, PERPHENAZINE, DOPAMINE, APOMORPHINE, PIMOZIDE, ARIPIPRAZOLE, RITANSERIN, CHLORPROTHIXENE, E-FLUPENTIXOL, TRIFLUOPERAZINE, DOMPERIDONE, Adoprazine, AMISULPRIDE, PERGOLIDE, CARIPRAZINE, FLUPENTIXOL, DRONABINOL, CLEMASTINE, KETANSERIN, LOXAPINE, ECOPIPAM, RALOXIFENE, TAMOXIFEN, CLOMIPHENE, ENCLOMIPHENE, IFENPRODIL, TRIPARANOL, TESEVATINIB, ROCILETINIB, CP-724714, AEE-788, CUDC-101, FALNIDAMOL, AZD-3759, DOCETAXEL, SAPITINIB, NAQUOTINIB, NAZARTINIB, OLMUTINIB, MAVELERTINIB, PF-06459988, DACOMITINIB, GEFITINIB, ICOTINIB, NERATINIB, TAK-285, MIDOSTAURIN, AFATINIB, VANDETANIB, LAPATINIB, ERLOTINIB, CANERTINIB, IBRUTINIB, POZIOTINIB, VARLITINIB, OSIMERTINIB, PELITINIB, SORAFENIB, BOSUTINIB, PF-04457845, MK-3168, ORLISTAT, URB-597, AT-9283, TAFETINIB, CEP-11981, LUCITANIB, KRN-633, MK-2461, TIVOZANIB, SEMAXANIB, AXITINIB, BRIVANIB, VATALANIB, CEDIRANIB, BRIGATINIB, CEP-5214, BEMCENTINIB, CABOZANTINIB, CRENOLANIB, PACRITINIB, PONATINIB, BARASERTIB, ENMD-2076, CEP-1347, R-406, TOZASERTIB, KW-2449, FEDRATINIB, NINTEDANIB, GILTERITINIB, TELATINIB, LENVATINIB, CHLORAMBUCIL, FIMEPINOSTAT, QUISINOSTAT, ABEXINOSTAT, Givinostat, NANATINOSTAT, PYROXAMIDE, ROMIDEPSIN, MOCETINOSTAT, ENTINOSTAT, TRICHOSTATIN, BELINOSTAT, R-306465, BENDAMUSTINE, RICOLINOSTAT, CITARINOSTAT, AR-42, MAPROTILINE, PYRILAMINE, METHAPYRILENE, AZELASTINE, MIRTAZAPINE, TRIPROLIDINE, GSK-1004723, MIZOLASTINE, RUPATADINE, AZATADINE, CYPROHEPTADINE, HYDROXYZINE, CINNARIZINE, DESLORATADINE, CYCLIZINE, EBASTINE, BENZTROPINE, DIMETHINDENE LEVOCETIRIZINE, CETIRIZINE, MEPAZINE, TERFENADINE, PROMETHAZINE, DEXCHLORPHENIRAMINE CHLORPHENIRAMINE, AMOXAPINE, KETOTIFEN, PU-H71, TANESPIMYCIN, LUMINESPIB, GANETESPIB, BIIB021, ALVESPIMYCIN, ALVESPIMYCIN, GELDANAMYCIN, TRAZODONE, PRUVANSERIN, NELOTANSERIN, VOLINANSERIN, TEGASEROD, VELUSETRAG, TEMANOGREL, SEROTONIN, LYSERGIDE, METHYSERGIDE, ERGONOVINE, NEFAZODONE, METHYLERGONOVINE, OXITRIPTAN, LORCASERIN, CABERGOLINE, SUMATRIPTAN, CHLOROPHENYLPIPERAZINE VABICASERIN, IDALOPIRDINE, CERLAPIRDINE, INTEPIRDINE, LANDIPIRDINE, LATREPIRDINE, JNJ-18038683, BMS-754807, CERITINIB, XL-228, LINSITINIB, AS-602868, DOFETILIDE, IBUTILIDE, HALOFANTRINE, VERAPAMIL, ANAGLIPTIN, CITALOPRAM, QUINIDINE, ESCITALOPRAM, QUININE, OSI-632, OSI-930, RIVOCERANIB, RG-1530, AG-13958, SITRAVATINIB, ALTIRATINIB, REGORAFENIB, CRIZOTINIB, SIROLIMUS, SARACATINIB, BAFETINIB, TALMAPIMOD, NEFLAMAPIMOD, QUERCETIN, CI-1040, DORAMAPIMOD, ARRY-797, PH-797804, VX-702, LOSMAPIMOD, TAK-715, PAMAPIMOD, R-1487, JNJ-49095397, CTS-1027, ZOTAROLIMUS, BGT-226, RG-7603, TACROLIMUS, EVEROLIMUS, PKI-179, AZD-8055, DACTOLISIB, GEDATOLISIB, OMIPALISIB, VISTUSERTIB, PF-04691502, APITOLISIB, SAPANISERTIB, BORTEZOMIB, IXAZOMIB, PD-0166285, RAF-265, TG100-115, TASELISIB, PICTILISIB, BUPARLISIB, COPANLISIB, ALPELISIB, BIMIRALISIB, AZD-8835, AZD-6482, GSK-2636771, NEMIRALISIB, GS-9901, LENIOLISIB, IDELALISIB, DUVELISIB, IPI-549, TAK-960, ONVANSERTIB, RIGOSERTIB, VOLASERTIB, GSK-461364, BI-2536, ADAVOSERTIB, LINOLEIC ACID, PEMAFIBRATE, NAMODENOSON, GW501516, INT131, EFATUTAZONE, MK-0533, ROSIGLITAZONE, MURAGLITAZAR, FARGLITAZAR, SEMAGACESTAT, BEGACESTAT, AVAGACESTAT, NIROGACESTAT, LIAFENSINE, NOMIFENSINE, 1-(3,4-DICHLOROPHENYL)-6-(METHOXYMETHYL)-3-AZABICYCLO[4.1.0]HEPTANE (ENANTIOMERIC MIX), ESREBOXETINE, REBOXETINE, DULOXETINE, ATOMOXETINE, MAZINDOL, AMPHETAMINE, DEXTROAMPHETAMINE, MILNACIPRAN, LEVOMILNACIPRAN, PROTRIPTYLINE, IOFLUPANE, FLUVOXAMINE, PYROVALERONE, VANOXERINE, PAROXETINE, LITOXETINE, AMITIFADINE, UK-390957, INDALPINE, VORTIOXETINE, VILAZODONE, MIDOMAFETAMINE, PRIMAQUINE, COCAINE, DEXTROMETHORPHAN, SERTRALINE, VENLAFAXINE, OZAGREL, TERBOGREL, DOLASTATIN-10, PATUPILONE, pharmaceutically acceptable salt of any one of the compounds of the table 1 ab and Table 1c, solvate of any one of the compounds of the table 1 ab and Table 1c, hydrate of any one of the compounds of the table 1 ab and Table 1c, tautomer of any one of the compounds of the table 1 ab and Table 1c, geometric isomer of any one of the compounds of the table 1 ab and Table 1c, optical isomer of any one of the compounds of the table 1 ab and Table 1c, stereoisomer of any one of the compounds of the table 1 ab and Table 1c, structural analog of any one of the compounds of the table 1 ab and Table 1c, functional analog of any one of the compounds of the table 1 ab and Table 1c, derivative of any one of the compounds of the table 1 ab and Table 1c, prodrug of any one of the compounds of the table 1 ab and Table 1c.

The application also provides an anti-aging therapy, comprising a compound selected from the group of all compounds of Table 1 ab and Table 1c.

In some embodiments, the application also provides an senolytic therapy, comprising a compound selected from the group of all compounds of Table 1 ab and Table 1c.

The application also provides an senostatic therapy, comprising a compound selected from the group of all compounds of Table 1 ab and Table 1c.

In some embodiments, the application also provides adjuvant tumour therapy, comprising a compound selected from the group of all compounds of Table 1 ab and Table 1c.

The application also provides an anti-aging therapy, comprising the compound selected from Table 1 ab and Table 1c and an enhancer moiety. Optionally, the enhancer moiety is a permeability enhancer, stability enhancer or bioavailability enhancer.

The application also provides a composition comprising a compound as described herein or a compound as described herein; and a carrier.

The application also provides a pharmaceutical composition comprising a compound as described herein; and a pharmaceutically acceptable carrier.

In some embodiments, any one of the inhibitors of any one of the Targets, including but not limited to any one of the compounds selected from Table 1 ab and Table 1c has at least one of the effects selected from the following group consisting of: ameliorates at least one symptom of the age-related disease or condition in the subject, delays mortality, delays onset of at least one aging associated diseases, decreases frailty, e.g. the Frailty Index (e.g. 31 phenotypes that are indicators of age-associated health deterioration), improves motor function, increases expression of PSD95 and synaptophysin (markers of pre- and post-synaptic density, respectively), or lowers the number of senescent cells in lymphocyte subpopulations

In some embodiments, the term “anti-aging” is used interchangeably with the term senolytic”. In some embodiments, the term “anti-aging therapy” is used interchangeably with the term “Senostatic”.

Accordingly, the application includes a use of a compound selected from Table 1 ab and Table 1c for treating or preventing of the condition selected from the group consisting of: aging, frailty, an age-related disease or condition in a subject; or for delaying or reversing one or more signs or symptoms of aging in a subject and/or for increasing longevity in a subject.

The application also provides a use of the inhibitor of Target, including but not limited to any one compound selected from Table 1 ab and Table 1c for treating or preventing a senescence associated disease or disorder in a subject and/or for selectively killing one or more senescent cells in a subject.

The application further provides a use of the compound selected from Table 1 ab and Table 1c for treating or preventing viral disease, optionally COVID-19 or influenza, or infectious disease, or pneumonia in a subject or for alleviating signs and symptoms of viral disease, optionally COVID-19 or influenza, or infectious disease in a subject.

In one embodiment, the anti-aging drug is a molecule that binds any at least one of the Targets or/and inhibits activity of such Target.

In another embodiment, the anti-aging therapy includes the compound selected from the Table 1 ab and Table 1c conjugated to an enhancer moiety or a composition comprising the compound or compound or an antibody or antibody fragment that binds target selected from the group of Targets.

In one embodiment, the age-related disease or condition is selected from the group consisting of age-related tissue decline, age-related organ decline, degenerative disease, function-decreasing disorder, cancer, Alzheimer's disease, Parkinson's disease, cataracts, macular degeneration, glaucoma, atherosclerosis, acute coronary syndrome, myocardial infarction, stroke, recovery after stroke, PSD-95 decrease, hypertension, idiopathic pulmonary fibrosis (IPF), chronic obstructive pulmonary disease (COPD), type 1 diabetes, obesity, fat dysfunction, coronary artery disease, cerebrovascular disease, periodontal disease, cancer treatment-related disability, chemotherapy treatment-related disability, chemotherapy treatment-related frailty, frailty, radiotherapy and other radiation related disability, chemotherapy treatment-related frailty, cancer treatment-related atrophy, cancer treatment-related fibrosis, brain injury, heart injury, and therapy-related myelodysplastic syndrome, accelerated aging, accelerated aging disease, Hutchinson-Gilford progeria syndrome, Werner syndrome, Cockayne syndrome, exroderma pigmentosum, ataxia telangiectasia, Fanconi anemia, dyskeratosis congenital, aplastic anemia, idiopathic pulmonary fibrosis, atherosclerosis, cardiovascular disease, adult cancer, arthritis, cataracts, osteoporosis, type 2 diabetes, diabetes, hypertension, neurodegeneration, stroke, atrophic gastritis, osteoarthritis, NASH, camptocormia, chronic obstructive pulmonary disease, coronary artery disease, dopamine dysregulation syndrome, metabolic syndrome, effort incontinence, Hashimoto's thyroiditis, heart failure, late life depression, immunosenescence, myocardial infarction, acute coronary syndrome, sarcopenia, sarcopenic obesity, senile osteoporosis and urinary incontinence.

In another embodiment, the subject is a mammal, optionally a human, optionally a human of at least 60, 65, 70, 75, 80, 85, 90, 95 or 100 years of age.

Other features and advantages of the present application will become apparent from the following detailed description. It should be understood, however, that the detailed description and the specific examples while indicating embodiments of the application are given by way of illustration only, since various changes and modifications within the spirit and scope of the application will become apparent to those skilled in the art from this detailed description.

In some embodiments, the wording “the compound selected from Table 1 ab and Table 1c” means “the compound selected from the group consisting of all compounds listed in the Table 1 ab and Table 1c”.

In some embodiments, the wording “the compound selected from Table 1 ab and Table 1c” or “the compound selected from the group consisting of all compounds listed in the Table 1 ab and Table 1c” means “the pharmaceutical composition selected from the group consisting of all pharmaceutical compositions listed in the Table 1 ab and Table 1c”.

In some embodiments, the wording “the compound selected from Table 1 ab and Table 1c” means compound selected from the group, consisting of: CYCLOSPORINE, TARIQUIDAR, MARIMASTAT, PRINOMASTAT, APRATASTAT, YOHIMBINE, QUETIAPINE, DOXEPIN, MIANSERIN, ERGOTAMINE, PIPAMAZINE, PHENTOLAMINE, LISURIDE, TAMSULOSIN, DEXMEDETOMIDINE, INDORAMIN, RISPERIDONE, SERTINDOLE, XYLOMETAZOLINE, NAPHAZOLINE, TETRAHYDROZOLINE, DESIPRAMINE, ALFUZOSIN, SILODOSIN, TERAZOSIN, OLANZAPINE, ZOTEPINE, DROPERIDOL, THIORIDAZINE, CHLORPROMAZINE, FLUPHENAZINE, CARVEDILOL, OXYMETAZOLINE, HALOPERIDOL, PHENOXYBENZAMINE, NAFTOPIDIL, DOXAZOSIN, BROMOCRIPTINE, CLOZAPINE, PRAZOSIN, DIHYDROERGOTAMINE, CLOMIPRAMINE, AMITRIPTYLINE, IMIPRAMINE, NORTRIPTYLINE, ASTEMIZOLE, PROMAZINE, ZIPRASIDONE, PHENYLEPHRINE, CISAPRIDE, DAPIPRAZOLE, ILOPERIDONE, MORPHINE, ZOLMITRIPTAN, LOFEXIDINE, FIPAMEZOLE, FLUOXETINE, ATIPAMEZOLE, APRACLONIDINE, LEVONORDEFRIN, IDAZOXAN, BRIMONIDINE, GUANABENZ, EPINEPHRINE, NOREPINEPHRINE, CLONIDINE, CLONIDINE, METERGOLINE, PROCHLORPERAZINE, LY-2811376, LANABECESTAT, AZD-3839, VERUBECESTAT, BIRABRESIB, MIVEBRESIB, VORINOSTAT, ALOBRESIB, AZD-5153, PANOBINOSTAT, ACETAZOLAMIDE, BRINZOLAMIDE, MAFENIDE, INDISULAM, INDAPAMIDE, DTP-348, TOPIRAMATE, LEVOSULPIRIDE, DORZOLAMIDE, SULPIRIDE, CHLORTHALIDONE, METHAZOLAMIDE, NILOTINIB, ETHOXZOLAMIDE, ZONISAMIDE, DICHLORPHENAMIDE, SULTHIAME, METOLAZONE, TRICHLORMETHIAZIDE, SACCHARIN, LUTEOLIN, ISOQUERCETIN, DAIDZEIN, FAMOTIDINE, AZD-5438, TRILACICLIB, RGB-286638, ZOTIRACICLIB, LEROCICLIB, PHA-793887, PALBOCICLIB, HEXAMETHYL PARAROSANILINE, RG-547, DINACICLIB, FORETINIB, AST-487, AT-7519, AZD-7762, UCN-01, SUNITINIB, SILMITASERTIB, LESTAURTINIB, EDICOTINIB, ILORASERTIB, PEXIDARTINIB, TAK-593, PAZOPANIB, TANDUTINIB, DOVITINIB, CEP-32496, QUIZARTINIB, LINIFANIB, MOTESANIB, MASITINIB, DASATINIB, IMATINIB, SU-014813, RELACATIB, BALICATIB, ODANACATIB, ROTIGOTINE, CLEBOPRIDE, RACLOPRIDE, SUMANIROLE, PRAMIPEXOLE, PF-00217830, PALIPERIDONE, BENPERIDOL, BREXPIPRAZOLE, BIFEPRUNOX, HALOPERIDOL DECANOATE, ARMODAFINIL, MODAFINIL, MESORIDAZINE, ASENAPINE, BLONANSERIN, CLOTHIAPINE, FALLYPRIDE, ROPINIROLE, SARIZOTAN, PERPHENAZINE, DOPAMINE, APOMORPHINE, PIMOZIDE, ARIPIPRAZOLE, RITANSERIN, CHLORPROTHIXENE, E-FLUPENTIXOL, TRIFLUOPERAZINE, DOMPERIDONE, Adoprazine, AMISULPRIDE, PERGOLIDE, CARIPRAZINE, FLUPENTIXOL, DRONABINOL, CLEMASTINE, KETANSERIN, LOXAPINE, ECOPIPAM, RALOXIFENE, TAMOXIFEN, CLOMIPHENE, ENCLOMIPHENE, IFENPRODIL, TRIPARANOL, TESEVATINIB, ROCILETINIB, CP-724714, AEE-788, CUDC-101, FALNIDAMOL, AZD-3759, DOCETAXEL, SAPITINIB, NAQUOTINIB, NAZARTINIB, OLMUTINIB, MAVELERTINIB, PF-06459988, DACOMITINIB, GEFITINIB, ICOTINIB, NERATINIB, TAK-285, MIDOSTAURIN, AFATINIB, VANDETANIB, LAPATINIB, ERLOTINIB, CANERTINIB, IBRUTINIB, POZIOTINIB, VARLITINIB, OSIMERTINIB, PELITINIB, SORAFENIB, BOSUTINIB, PF-04457845, MK-3168, ORLISTAT, URB-597, AT-9283, TAFETINIB, CEP-11981, LUCITANIB, KRN-633, MK-2461, TIVOZANIB, SEMAXANIB, AXITINIB, BRIVANIB, VATALANIB, CEDIRANIB, BRIGATINIB, CEP-5214, BEMCENTINIB, CABOZANTINIB, CRENOLANIB, PACRITINIB, PONATINIB, BARASERTIB, ENMD-2076, CEP-1347, R-406, TOZASERTIB, KW-2449, FEDRATINIB, NINTEDANIB, GILTERITINIB, TELATINIB, LENVATINIB, CHLORAMBUCIL, FIMEPINOSTAT, QUISINOSTAT, ABEXINOSTAT, Givinostat, NANATINOSTAT, PYROXAMIDE, ROMIDEPSIN, MOCETINOSTAT, ENTINOSTAT, TRICHOSTATIN, BELINOSTAT, R-306465, BENDAMUSTINE, RICOLINOSTAT, CITARINOSTAT, AR-42, MAPROTILINE, PYRILAMINE, METHAPYRILENE, AZELASTINE, MIRTAZAPINE, TRIPROLIDINE, GSK-1004723, MIZOLASTINE, RUPATADINE, AZATADINE, CYPROHEPTADINE, HYDROXYZINE, CINNARIZINE, DESLORATADINE, CYCLIZINE, EBASTINE, BENZTROPINE, DIMETHINDENE LEVOCETIRIZINE, CETIRIZINE MEPAZINE, TERFENADINE, PROMETHAZINE, DEXCHLORPHENIRAMINE, CHLORPHENIRAMINE, AMOXAPINE, KETOTIFEN, PU-H71, TANESPIMYCIN, LUMINESPIB, GANETESPIB, BIIB021, ALVESPIMYCIN, ALVESPIMYCIN, GELDANAMYCIN, TRAZODONE, PRUVANSERIN, NELOTANSERIN, VOLINANSERIN, TEGASEROD, VELUSETRAG, TEMANOGREL, SEROTONIN, LYSERGIDE, METHYSERGIDE, ERGONOVINE, NEFAZODONE, METHYLERGONOVINE, OXITRIPTAN, LORCASERIN, CABERGOLINE, SUMATRIPTAN, CHLOROPHENYLPIPERAZINE, VABICASERIN, IDALOPIRDINE, CERLAPIRDINE, INTEPIRDINE, LANDIPIRDINE, LATREPIRDINE, JNJ-18038683, BMS-754807, CERITINIB, XL-228, LINSITINIB, AS-602868, DOFETILIDE, IBUTILIDE, HALOFANTRINE, VERAPAMIL, ANAGLIPTIN, CITALOPRAM, QUINIDINE, ESCITALOPRAM, QUININE, OSI-632, OSI-930, RIVOCERANIB, RG-1530, AG-13958, SITRAVATINIB, ALTIRATINIB, REGORAFENIB, CRIZOTINIB, SIROLIMUS, SARACATINIB, BAFETINIB, TALMAPIMOD, NEFLAMAPIMOD, QUERCETIN, CI-1040, DORAMAPIMOD, ARRY-797, PH-797804, VX-702, LOSMAPIMOD, TAK-715, PAMAPIMOD, R-1487, JNJ-49095397, CTS-1027, ZOTAROLIMUS, BGT-226, RG-7603, TACROLIMUS, EVEROLIMUS, PKI-179, AZD-8055, DACTOLISIB, GEDATOLISIB, OMIPALISIB, VISTUSERTIB, PF-04691502, APITOLISIB, SAPANISERTIB, BORTEZOMIB, IXAZOMIB, PD-0166285, RAF-265, TG100-115, TASELISIB, PICTILISIB, BUPARLISIB, COPANLISIB, ALPELISIB, BIMIRALISIB, AZD-8835, AZD-6482, GSK-2636771, NEMIRALISIB, GS-9901, LENIOLISIB, IDELALISIB, DUVELISIB, IPI-549, TAK-960, ONVANSERTIB, RIGOSERTIB, VOLASERTIB, GSK-461364, BI-2536, ADAVOSERTIB, LINOLEIC ACID, PEMAFIBRATE, NAMODENOSON, GW501516, INT131, EFATUTAZONE, MK-0533, ROSIGLITAZONE, MURAGLITAZAR, FARGLITAZAR, SEMAGACESTAT, BEGACESTAT, AVAGACESTAT, NIROGACESTAT, LIAFENSINE, NOMIFENSINE, 1-(3,4-DICHLOROPHENYL)-6-(METHOXYMETHYL)-3-AZABICYCLO[4.1.0]HEPTANE (ENANTIOMERIC MIX), ESREBOXETINE, REBOXETINE, DULOXETINE, ATOMOXETINE, MAZINDOL, AMPHETAMINE, DEXTROAMPHETAMINE, MILNACIPRAN, LEVOMILNACIPRAN, PROTRIPTYLINE, IOFLUPANE, FLUVOXAMINE, PYROVALERONE, VANOXERINE, PAROXETINE, LITOXETINE, AMITIFADINE, UK-390957, INDALPINE, VORTIOXETINE, VILAZODONE, MIDOMAFETAMINE, PRIMAQUINE, COCAINE, DEXTROMETHORPHAN, SERTRALINE, VENLAFAXINE, OZAGREL, TERBOGREL, DOLASTATIN-10, PATUPILONE, pharmaceutically acceptable salt of any one of the compounds of the table 1 ab and Table 1c, solvate of any one of the compounds of the table 1 ab and Table 1c, hydrate of any one of the compounds of the table 1 ab and Table 1c, tautomer of any one of the compounds of the table 1 ab and Table 1c, geometric isomer of any one of the compounds of the table 1 ab and Table 1c, optical isomer of any one of the compounds of the table 1 ab and Table 1c, stereoisomer of any one of the compounds of the table 1 ab and Table 1c, structural analog of any one of the compounds of the table 1 ab and Table 1c, functional analog of any one of the compounds of the table 1 ab and Table 1c, derivative of any one of the compounds of the table 1 ab and Table 1c, prodrug of any one of the compounds of the table 1 ab and Table 1c.

In some embodiments, the wording “the compound selected from Table 1 ab and Table 1c” or “the compound selected from the group consisting of all compounds listed in the Table 1 ab and Table 1c” means Active pharmaceutical ingredient (API), selected from the group consisting of all Active pharmaceutical ingredients of all of the drugs, drug candidates, pharmaceutical compositions and other agents listed in the Table 1 ab and Table 1c.

In some embodiments, the wordings “the compound selected from Table 1 ab and Table 1c”, “the compound selected from the group consisting of all compounds listed in the Table 1 ab and Table 1c”, “the pharmaceutical composition selected from the group consisting of all pharmaceutical compositions listed in the Table 1 ab and Table 1c” and alike can be used interchangeably with the wording “the agent reducing, inhibiting, or degrading a protein selected from the group consisting of: HTR2A, SLC6A3, SLC6A2, SCN2A, BMX, ADRA1B, FGFR1, EGFR, HRH1, HSP90AA1, HTR2C, CNR1, HDAC10, ERBB2, MAP3K19, BRD2, BRD4, PIK3CD, NR3C1, HTR2B, ERBB3, BLK, KDR, EPHA8, ADRA1A, EPHA6, HDAC9, PIK3CA, FLT3, ERBB4, DRD5, PIK3R1, HTR7, HDAC1, SCN3A, EBP, BRD3, DRD3, HDAC4, HDAC2, STK24, HDAC8, HDAC6, SCN1A, FLT1, IGF1R, HDAC11, MAP4K5, KCNH2, HDAC7, STK10, HMGCR, SLC6A4, ABL1, PDGFRB, HDAC3, LCK, ADRA1D, HTR6, DRD2, KIT, HDAC5, DDR1, ABL2.

In some embodiments, compound selected from the table 1 ab and Table 1c is used for Simultaneous Alleviation of Co-morbidities. For example, in elderly subjects with multi-morbidity, a compound selected from the table 1 ab and Table 1c alleviates one or more of: mild diabetes, atherosclerosis, hypertension, mild cognitive impairment, sarcopenia, osteoarthritis, mild renal insufficiency, etc. occurring within a subject. In some embodiments, a compound selected from the table 1 ab and Table 1c is used for improving parameter selected from the group consisting of: glucose tolerance, carotid flow velocity, blood pressure, timed walking ability, walking speed, joint pain, joint pain inventories, creatinine clearance. In such a scenario, short-term outcomes such as glucose tolerance tests, carotid flow velocity, blood pressure, timed walking ability, joint pain inventories, and creatinine clearance could be measured following administration of the compound selected from the table 1 ab and Table 1c or placebo.

In some embodiments, compound selected from the table 1 ab and Table 1c is used for Delaying Accelerated Aging-like Conditions. In some embodiments, compound selected from the table 1 ab and Table 1c is used for one of the selected from the group: treatment of the frailty, improving of at least one of endurance parametered, improving of at least one of metabolic parameters, improving of at least one of cardiovascular functions, improving of at least one of cognitive functions, alleviating at least one symptom in childhood cancer survivors, alleviating at least one symptom caused by chemotherapy in childhood cancer survivors, alleviating at least one symptom caused by chemotherapy, alleviating at least one symptom caused by radiotherapy, alleviating at least one symptom in bone marrow transplant survivors, alleviating at least one symptom caused by bone marrow transplantation, alleviating at least one progeroid syndrome, patients alleviating at least one symptom of diabetes due to obesity, alleviating at least one symptom of diabetes, alleviating at least one symptom of HIV or AIDS, alleviating at least one of conditions related to latent viruses, such as HIV.

In this scenario, positive effects of a compound selected from the table 1 ab and Table 1c on frailty and endurance measures, metabolic parameters, assays of cardiovascular and cognitive function, and other outcomes could be used in: childhood cancer survivors, bone marrow transplant survivors, subjects with progeroid syndromes, patients with diabetes due to obesity, or in conditions related to latent viruses, such as HIV. In some embodiments, for all clinical trials scenarios, assays of senescent cell burden would be needed.

In some embodiments, compound selected from the table 1 ab and Table 1c is used for treating Conditions with Localized Cellular Senescence. In some embodiments, to kill senescent cells selectively, high local concentrations could be achieved by injections, drops, aerosols, or topical solutions. In some embodiments, compound selected from the table 1 ab and Table 1c is used for treating disease selected from the group consisting of: osteoarthritis, fracture non-union, glaucoma and macular degeneration, sites subjected to therapeutic radiation, the lungs in idiopathic pulmonary fibrosis, chronic obstructive pulmonary disease, or damage due to tobacco, or atherosclerotic plaques (by catheterization), among others.

In some embodiments, compound selected from the table 1 ab and Table 1c is used for treating of Otherwise Fatal Conditions, Such conditions include: idiopathic pulmonary fibrosis, primary sclerosing cholangitis, cancers, or HIV dementia, among others.

In some embodiments, compound selected from the table 1 ab and Table 1c is used for increasing Resilience or Clinical Stresses in Pre-frail Subjects. Capacity to recover following a medical or physiological stress generally declines with aging. In some embodiments, compound selected from the table 1 ab and Table 1c is used to increase resilience. An example might include the use of such agents before chemotherapy in an effort to improve recovery and to allow providing higher, more effective doses of chemotherapy to elderly frail subjects with high initial burdens of senescent cells. In some embodiments, compound selected from the table 1 ab and Table 1c is used for targeting basic aging processes, to accelerate recovery after elective surgery, bone marrow transplantation, therapeutic radiation, pneumonia, or myocardial infarction or to enhance immune response to influenza vaccination. In some embodiments, compound selected from the table 1 ab and Table 1c is used for two weeks before influenza vaccination results in better influenza antibody generation in elderly subjects (for example, the same protocol as described Mannick et al., 2014). In some embodiments, compound selected from the table 1 ab and Table 1c administered as a single course before vaccination is used for the same.

In some embodiments, compound selected from the table 1 ab and Table 1c is used for reducing frailty, including use to alleviate slow gait, decreased strength, or sarcopenia or delay loss of independence. In some embodiments, compound selected from the table 1 ab and Table 1c is used for reducing frailty in progeroid patients In other particular embodiments, a method is provided for treating, reducing the likelihood of occurrence of, or delaying onset of a senescent cell-associated disease or disorder in a subject who has a senescent cell-associated disease or disorder or who has at least one predisposing factor for developing the senescent cell-associated disease or disorder, comprising administering to the subject a compound selected from the table 1 ab and Table 1c that alters either one or both of a cell survival signaling pathway and an inflammatory pathway in the senescent cell, thereby promoting death of the senescent cell, with the proviso that if the subject has a cancer, the compound selected from the table 1 ab and Table 1c is not a primary therapy for treating the cancer, wherein the compound selected from the table 1 ab and Table 1c is administered once every 0.5-12 months, and wherein the senescent cell-associated disease or disorder is a cardiovascular disease or disorder, inflammatory disease or disorder, a pulmonary disease or disorder, a neurological disease or disorder, a chemotherapeutic side effect, a radiotherapy side effect, or metastasis. In another specific embodiment, a method is provided for treating, reducing the likelihood of occurrence of, or delaying onset of a senescent cell-associated disease or disorder in a subject who has a senescent cell-associated disease or disorder or who has at least one predisposing factor for developing the senescent cell-associated disease or disorder, comprising administering to the subject a compound selected from the table 1 ab and Table 1c that alters either one or both of a cell survival signaling pathway and an inflammatory pathway in the senescent cell, thereby promoting death of the senescent cell, wherein the compound selected from the table 1 ab and Table 1c is administered once every 4-12 months.

The treatment regimen of the methods for treating a senescence associated disease or disorder, comprises administering a compound selected from the table 1 ab and Table 1c for a time sufficient and in an amount sufficient that selectively kills senescent cells. In certain embodiments, the compound selected from the table 1 ab and Table 1c is administered within a treatment cycle, which treatment cycle comprises a treatment course followed by a non-treatment interval. A treatment course of administration refers herein to a finite time frame over which one or more doses of the compound selected from the table 1 ab and Table 1c on one or more days are administered. The finite time frame may be also called herein a treatment window.

In one embodiment, a method is provided herein for treating a senescence-associated disease or disorder, which is not a cancer, and which method comprises administering to a subject in need thereof a small molecule compound selected from the table 1 ab and Table 1c that selectively kills senescent cells and is administered within a treatment cycle. In a particular embodiment, the methods comprise administering the compound selected from the table 1 ab and Table 1c in at least two treatment cycles. In a specific embodiment, the non-treatment interval may be at least about 2 weeks or between at least about 0.5-12 months, such as at least about one month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months (i.e., 1 year). In other certain particular embodiments, the non-treatment interval is between 1-2 years or between 1-3 years, or longer. In certain embodiments, each treatment course is no longer than about 1 month, no longer than about 2 months, or no longer than about 3 months; or is no longer than 1, 2, 3, 4, 5, 6, 7, 8, 9, 10, 11, 12, 13, 14, 15, 16, 17, 18, 19, 20, 21, 22, 23, 24, 25, 26, 26, 27, 28, 29, 30, or 31 days.

In certain embodiments, the treatment window (i.e., treatment course) is only one day. In other certain embodiments, a single treatment course occurs over no longer than 2, 3, 4, 5, 6, 7, 8, 9, 10, 11, 12, 13, 14, 15, 16, 17, 18, 19, 20, 21, 22, 23, 24, 25, 26, 26, 27, 28, 29, 30, or 31 days, During such treatment windows, the compound selected from the table 1 ab and Table 1c may be administered at least on two days (i.e., two days or more) with a variable number of days on which the agent is not administered between the at least two days of administration. Stated another way, within a treatment course when the compound selected from the table 1 ab and Table 1c is administered on two or more days, the treatment course may have one or more intervals of one or more days when the Compound selected from the table 1 ab and Table 1c, is not administered. By way of non-limiting example, when the compound selected from the table 1 ab and Table 1c is administered on 2 or more days during a treatment course not to exceed 21 days, the agent may be administered on any total number of days between from 2, 3, 4, 5, 6, 7, 8, 9, 10, 11, 12, 13, 14, 15, 16, 17, 18, 19, 20, 21, 22, 23, 24, 25, 26, 26, 27, 28, 29, 30, or 31 days. In certain embodiments, the compound selected from the table 1 ab and Table 1c is administered to a subject during a treatment course of 3 days or more, and the agent may be administered every 2^{nd} day (i.e., every other day). In other certain embodiments when the compound selected from the table 1 ab and Table 1c is administered to a subject for a treatment window of 4 days or more, the compound selected from the table 1 ab and Table 1c may be administered every 3^{rd} day (i.e., every other third day). In one embodiment, the compound selected from the table 1 ab and Table 1c is administered on at least two days (i.e., 2 or more) during a treatment course that is at least 2 days and no more than about 21 days (i.e., from about 2-21 days); at least 2 days and no longer than about 14 days (i.e., from about 2-14 days); at least 2 days and no longer than about 10 days (i.e., from about 2-10 days); or at least 2 days and no longer than about 9 days (i.e., from about 2-9 days); or at least 2 days and no longer than about 8 days (i.e., from about 2-8 days). In other specific embodiments, the compound selected from the table 1 ab and Table 1c is administered on at least two days (i.e., 2 or more) during a treatment window is at least 2 days and no longer than about 7 days (i.e., from about 2-7 days); at least 2 days and no longer than about 6 days (i.e., from about 2-6 days) or at least 2 days and no more than about 5 days (i.e., from about 2-5 days) or at least 2 days and no longer than about 4 days (i.e., from about 2-4 days). In yet another embodiment, the treatment window is at least 2 days and no longer than 3 days (i.e., 2-3 days), or 2 days. In certain particular embodiments, the treatment course is no longer than 3 days. In other embodiments, the treatment course is no longer than 5 days. In still other specific embodiments, the treatment course is no longer than 7 days, 10 days, or 14 days or 21 days. In certain embodiments, the compound selected from the table 1 ab and Table 1c is administered on at least two days (i.e., 2 or more days) during a treatment window that is at least 2 days and no longer than about 11 days (i.e., 2-11 days); or the compound selected from the table 1 ab and Table 1c is administered on at least two days (i.e., 2 or more days) during a treatment window that is at least 2 days and no longer than about 12 days (i.e., 2-12 days); or the compound selected from the table 1 ab and Table 1c is administered on at least two days (i.e., 2 or more days) during a treatment window that is at least 2 days and no more than about 13 days (i.e., 2-13 days); or the compound selected from the table 1 ab and Table 1c is administered on at least two days (i.e., 2 or more days) during a treatment course that is at least 2 days and no more than about 15 days (i.e., 2-15 days); or the compound selected from the table 1 ab and Table 1c is administered on at least two days (i.e., 2 or more days) during a treatment course that is at least 2 days and no longer than about 16 days, 17 days, 18 days, 19 days, or 20 days (i.e., 2-16, 2-17, 2-18, 2-19, 2-20 days, respectively). In other embodiments, the compound selected from the table 1 ab and Table 1c may be administered on at least 3 days over a treatment course of at least 3 days and no longer than any number of days between 3 and 21 days; or is administered on at least 4 days over a treatment course of at least 4 days and no longer than any number of days between 4 and 21 days; or is administered on at least 5 days over a treatment course of at least 5 days and no longer than any number of days between 5 and 21 days; or is administered on at least 6 days over a treatment course of at least 6 days and no longer than any number of days between 6 and 21 days; or is administered at least 7 days over a treatment course of at least 7 days and no longer than any number of days between 7 and 21 days; or is administered at least 8 or 9 days over a treatment course of at least 8 or 9 days, respectively, and no longer than any number of days between 8 or 9 days, respectively, and 21 days; or is administered at least 10 days over a treatment course of at least 10 days and no longer than any number of days between 10 and 21 days; or is administered at least 14 days over a treatment course of at least 14 days and no longer than any number of days between 14 and 21 days; or is administered at least 11 or 12 days over a treatment course of at least 11 or 12 days, respectively, and no longer than any number of days between 11 or 12 days, respectively, and 21 days; or is administered at least 15 or 16 days over a treatment course of at least 15 or 16 days, respectively, and no longer than any number of days between 15 or 16 days, respectively, and 21 days. By way of additional example, when the treatment course is no longer than 14 days, a compound selected from the table 1 ab and Table 1c may be administered on at least 2, 3, 4, 5, 6, 7, 8, 9, 10, 11, 12, 13, and 14 days over a treatment of window of at least 2, 3, 4, 5, 6, 7, 8, 9, 10, 11, 12, 13, and 14 days, respectively, and no longer than 14 days. When the treatment course is no longer than 10 days, a compound selected from the table 1 ab and Table 1c may be administered on at least 2, 3, 4, 5, 6, 7, 8, 9, or 10 days over a treatment of window of at least 2, 3, 4, 5, 6, 7, 8, 9, or 10 days, respectively, and no longer than 10 days. Similarly, when the treatment course is no longer than 7 days, a compound selected from the table 1 ab and Table 1c may be administered on at least 2, 3, 4, 5, 6, or 7 days over a treatment window of at least 2, 3, 4, 5, 6, or 7 days, respectively, and no longer than 7 days. In still another example, when the treatment course is no longer than 5 days, a compound selected from the table 1 ab and Table 1c may be administered on at least 2, 3, 4, or 5 days over a treatment of window of at least 2, 3, 4, or 5 days, respectively, and no longer than 5 days.

With respect to a treatment course of three or more days, doses of the compound selected from the table 1 ab and Table 1c may be administered for a lesser number of days than the total number of days within the particular treatment window. By way of non-limiting example, when a course of treatment has a treatment course of no more than 7, 10, 14, or 21 days, the number of days on which the compound selected from the table 1 ab and Table 1c may be administered is any number of days between 2 days and 7, 10, 14, or 21 days, respectively, and at any interval appropriate for the particular disease being treated, the compound selected from the table 1 ab and Table 1c being administered, the health status of the patient and other relevant factors, which are discussed in greater detail herein. A person skilled in the art will readily appreciate that when the compound selected from the table 1 ab and Table 1c is administered on two or more days over a treatment window, the agent may be delivered on the minimum number days of the window, the maximum number of days of the window, or on any number of days between the minimum and the maximum.

In certain specific embodiments, a treatment course is one day or the treatment course is of a length not to exceed 2, 3, 4, 5, 6, 7, 8, 9, 10, 11, 12, 13, or 14 days, which are examples of a course wherein the compound selected from the table 1 ab and Table 1c is administered on two or more days over a treatment course not to exceed (i.e., no longer than) 2, 3, 4, 5, 6, 7, 8, 9, 10, 11, 12, 13, or 14 days, respectively. In other certain embodiments, the treatment course is about 2 weeks (about 14 days or 0.5 months), about 3 weeks (about 21 days), about 4 weeks (about one month), about 5 weeks, about 6 weeks (about 1.5 months), about 2 months (or about 60 days), or about 3 months (or about 90 days). In a particular embodiment, a treatment course is a single daily dosing of the Compound selected from the table 1 ab and Table 1c. In other embodiments, with respect to any treatment course a daily dose of the compound selected from the table 1 ab and Table 1c may be as a single administration or the dose may be divided into 2, 3, 4, or 5 separate administrations to provide the total daily dose of the agent.

As described herein, in certain specific embodiments, within a treatment window when the compound selected from the table 1 ab and Table 1c is administered on two are more days, the treatment course may have one or more intervals of one or more days when the Compound selected from the table 1 ab and Table 1c, is not administered. Solely as a non-limiting example, when a treatment window is between two and seven days, a first dose may be administered on the first day of the treatment window and a second dose may be administered on the third day of the course, and a third dose may be administered on the seventh day of the treatment window. A person skilled in the art will appreciate that varying dosing schedules may be used during a particular treatment window. In other specific embodiments, the compound selected from the table 1 ab and Table 1c is administered daily on each consecutive day for the duration of the treatment course. A daily dose may be administered as a single dose or the daily dose may be divided into 2, 3, or 4, or 5 separate administrations to provide the total daily dose of the Compound selected from the table 1 ab and Table 1c.

In certain embodiments, the treatment course comprises a length of time during which the compound selected from the table 1 ab and Table 1c is administered daily. In one specific embodiment, the compound selected from the table 1 ab and Table 1c is administered daily for 2 days. In another specific embodiment, the compound selected from the table 1 ab and Table 1c is administered daily for 3 days. In yet another particular embodiment, the compound selected from the table 1 ab and Table 1c is administered daily for 4 days. In one specific embodiment, the compound selected from the table 1 ab and Table 1c is administered daily for 5 days. In yet another particular embodiment, the compound selected from the table 1 ab and Table 1c is administered daily for 6 days. In another specific embodiment, the compound selected from the table 1 ab and Table 1c is administered daily for 7 days. In yet another particular embodiment, the compound selected from the table 1 ab and Table 1c is administered daily for 8 days. In still another specific embodiment, the compound selected from the table 1 ab and Table 1c is administered daily for 9 days. In yet another particular embodiment, the compound selected from the table 1 ab and Table 1c is administered daily for 10 days. In yet another particular embodiment, the compound selected from the table 1 ab and Table 1c is administered daily for 11 days. In yet another particular embodiment, the compound selected from the table 1 ab and Table 1c is administered daily for 12 days. In yet another particular embodiment, the compound selected from the table 1 ab and Table 1c is administered daily for 13 days. In yet another particular embodiment, the compound selected from the table 1 ab and Table 1c is administered daily for 14 days. The treatment window (i.e., course) for each of the above examples is no longer than 2, 3, 4, 5, 6, 7, 8, 9, 10, 11, 12, 13, 14 days, respectively.

In other specific embodiments, the compound selected from the table 1 ab and Table 1c is administered every 2^{nd} day (i.e., every other day) for 3, 4, 5, 6, 7, 8, 9, 10, 11, 12, 13, or 14 days. In still other specific embodiments, the compound selected from the table 1 ab and Table 1c is administered every 3^{rd} day (i.e., one day receiving the agent followed by two days without receiving the agent) for 4, 5, 6, 7, 8, 9, 10, 11, 12, 13, or 14 days. In still other specific embodiments, the compound selected from the table 1 ab and Table 1c may be administered on every 2^{nd}-3^{rd} day during a treatment window of 3, 4, 5, 6, 7, 8, 9, 10, 11, 12, 13, or 14 days. In yet other embodiments, the compound selected from the table 1 ab and Table 1c may be administered every 4^{th} day during a treatment course of 5, 6, 7, 8, 9, 10, 11, 12, 13, or 14 days; or every 5^{th} day during a treatment course of 6, 7, 8, 9, 10, 11, 12, 13, or 14 days. A person skilled in the art can readily appreciate the minimum numbers of days in a treatment window when the compound selected from the table 1 ab and Table 1c is administered every 6^{th}, 7^{th}, etc. day over a treatment window of a finite number of days as described herein.

In certain particular embodiments, a compound selected from the table 1 ab and Table 1c may be administered daily for a longer duration than 14 days and may be administered at least 15, 16, 17, 18, 19, 20, or at least 21 days. In other specific embodiments, the compound selected from the table 1 ab and Table 1c may be administered daily on each of the 15, 16, 17, 18, 19, 20, or 21 days. In another specific embodiment, the compound selected from the table 1 ab and Table 1c may be administered every second day during a treatment window of 15, 16, 17, 18, 19, 20, or 21 days. In another specific embodiment, the compound selected from the table 1 ab and Table 1c may be administered every third day during a treatment window of 15, 16, 17, 18, 19, 20, or 21 days. In still other specific embodiments, the compound selected from the table 1 ab and Table 1c may be administered on every 2^{nd}-3^{rd} day during a treatment window of 15, 16, 17, 18, 19, 20, or 21 days. In yet other embodiments, the compound selected from the table 1 ab and Table 1c may be administered every 4th day during a treatment course of 15, 16, 17, 18, 19, 20, or 21 days; or every 5^{th} day during a treatment course of 15, 16, 17, 18, 19, 20, or 21 days. A person skilled in the art can readily appreciate the minimum numbers of days in a treatment window when the compound selected from the table 1 ab and Table 1c is administered every 6^{th}, 7^{th}, etc. day over a treatment window of a finite number of days as described herein.

In another certain particular embodiment, a compound selected from the table 1 ab and Table 1c may be administered daily for a longer duration than 14 days and may be administered at least 15, 16, 17, 18, 19, 20, or at least 21 days. In other specific embodiments, the compound selected from the table 1 ab and Table 1c may be administered daily on each of the 15, 16, 17, 18, 19, 20, or 21 days. In another specific embodiment, the compound selected from the table 1 ab and Table 1c may be administered every second day during a treatment window of 15, 16, 17, 18, 19, 20, or 21 days. In another specific embodiment, the compound selected from the table 1 ab and Table 1c may be administered every third day during a treatment window of 15, 16, 17, 18, 19, 20, or 21 days. In still other specific embodiments, the compound selected from the table 1 ab and Table 1c may be administered on every 2^{nd}-3^{rd} day during a treatment window of 15, 16, 17, 18, 19, 20, or 21 days. In yet other embodiments, the compound selected from the table 1 ab and Table 1c may be administered every 4^{th} day during a treatment course of 15, 16, 17, 18, 19, 20, or 21 days; or every 5^{th} day during a treatment course of 15, 16, 17, 18, 19, 20, or 21 days. A person skilled in the art can readily appreciate the minimum numbers of days in a treatment window when the compound selected from the table 1 ab and Table 1c is administered every 6^{th}, 7^{th}, etc. day over a treatment window of a finite number of days as described herein.

In another certain particular embodiment, a compound selected from the table 1 ab and Table 1c may be administered in a treatment course daily for a longer duration than 14 days or 21 days and may be administered in a treatment course of about one month, about two months, or about three months. In other specific embodiments, the compound selected from the table 1 ab and Table 1c may be administered daily on each of a one month, two month, or three month treatment course. In another specific embodiment, the compound selected from the table 1 ab and Table 1c may be administered every second day during a treatment course of about one month, about two months, or about three months. In another specific embodiment, the compound selected from the table 1 ab and Table 1c may be administered every third day during a treatment course of about one month, about two months, or about three months. In still other specific embodiments, the compound selected from the table 1 ab and Table 1c may be administered on every 2^{nd}-3^{rd} day during a treatment course of about one month, about two months, or about three months. In yet other embodiments, the compound selected from the table 1 ab and Table 1c may be administered every 4^{th} day during a treatment course of about one month, about two months, or about three months; or every 5^{th} day during a treatment course of about one month, about two months, or about three months s. A person skilled in the art can readily appreciate the minimum numbers of days in a treatment course when the compound selected from the table 1 ab and Table 1c is administered every 6^{th}, 7^{th}, etc. day over a treatment window of a finite number of days as described herein.

By way of non-limiting example, a longer treatment window with a decreased dose per day may be a treatment option for a subject. In other particular embodiments and by way of example, the stage or severity of the senescence associated disease or disorder or other clinical factor may indicate that a longer term course may provide clinical benefit. In certain embodiments, the compound selected from the table 1 ab and Table 1c is administered daily, or optionally, every other day (every 2^{nd} day) or every 3^{rd} day, or greater interval (i.e., every 4^{th} day, 5^{th} day, 6^{th} day) during a treatment course of about 1-2 weeks (e.g., about 5-14 days), about 1-3 weeks (e.g., about 5-21 days), about 1-4 weeks (e.g., about 5-28 days, about 5-36 days, or about 5-42 days, 7-14 days, 7-21 days, 7-28 days, 7-36 days, or 7-42 days; or 9-14 days, 9-21 days, 9-28 days, 9-36 days, or 9-42 days. In other certain embodiments, the treatment course is between about 1-3 months. In a specific embodiment, the compound selected from the table 1 ab and Table 1c is administered daily for at least five days, and in another particular embodiment, the compound selected from the table 1 ab and Table 1c is administered daily for 5-14 days. In other particular embodiments, the compound selected from the table 1 ab and Table 1c is administered for at least seven days, for example, for 7-14, 7-21, 7-28 days, 7-36 days, or 7-42 days. In other particular embodiments, the compound selected from the table 1 ab and Table 1c is administered for at least nine days, for example, for 9-14 days, 9-21 days, 9-28 days, 9-36 days, or 9-42 days.

Even though as discussed herein and above, a treatment course comprising administering a compound selected from the table 1 ab and Table 1c provides clinical benefit, in other certain embodiments, a treatment course is repeated with a time interval between each treatment course when the compound selected from the table 1 ab and Table 1c is not administered (i.e., non-treatment interval, off-drug treatment). A treatment cycle as described herein and in the art comprises a treatment course followed by a non-treatment interval. A treatment cycle may be repeated as often as needed. For example, a treatment cycle may be repeated at least once, at least twice, at least three times, at least four times, at least five times, or more often as needed. In certain specific embodiments, a treatment cycle is repeated once (i.e., administration of the compound selected from the table 1 ab and Table 1c comprises 2 treatment cycles). In other certain embodiments, the treatment cycle is repeated twice or repeated 3 or more times. Accordingly, in certain embodiments, one, two, three, four, five, six, seven, eight, nine, ten, or more treatment cycles of treatment with a compound selected from the table 1 ab and Table 1c are performed. In particular embodiments, a treatment course or a treatment cycle may be repeated, such as when the senescence associated disease or disorder recurs, or when symptoms or sequelae of the disease or disorder that were significantly diminished by one treatment course as described above have increased or are detectable, or when the symptoms or sequelae of the disease or disorder are exacerbated, a treatment course may be repeated. In other embodiments when the compound selected from the table 1 ab and Table 1c is administered to a subject to prevent (i.e., reduce likelihood of occurrence or development) or to delay onset, progression, or severity of senescence associated disease or disorder, a subject may receive the compound selected from the table 1 ab and Table 1c over two or more treatment cycles. Accordingly, in certain embodiments, one cycle of treatment is followed by a subsequent cycle of treatment. Each treatment course of a treatment cycle or each treatment course of two or more treatment cycles are typically the same in duration and dosing of the Compound selected from the table 1 ab and Table 1c. In other embodiments, the duration and dosing of the compound selected from the table 1 ab and Table 1c during each treatment course of a treatment cycle may be adjusted as determined by a person skilled in the medical art depending, for example, on the particular disease or disorder being treated, the compound selected from the table 1 ab and Table 1c being administered, the health status of the patient and other relevant factors, which are discussed in greater detail herein. Accordingly, a treatment course of a second or any subsequent treatment cycle may be shortened or lengthened as deemed medically necessary or prudent. In other words, as would be appreciated by a person skilled in the art, each treatment course of two or more treatment cycles are independent and the same or different; and each non-treatment interval of each treatment cycle is independent and the same or different.

As described herein, in some embodiments, each course of treatment in a treatment cycle is separated by a time interval of days, weeks, or months without treatment with a compound selected from the table 1 ab and Table 1c (i.e., non-treatment time interval or off-drug interval; called non-treatment interval herein). The non-treatment interval (such as days, weeks, months) between one treatment course and a subsequent treatment course is typically greater than the longest time interval (i.e., number of days) between any two days of administration in the treatment course. By way of example, if a treatment course is no longer than 14 days and the agent is administered every other day during this treatment course, the non-treatment interval between two treatment courses is greater than 2 days, such as 3, 4, 5, 6, 7, 8, 9, 10, 11, 12, 13, or 14 days or about 3 weeks, about 4 weeks, about 6 weeks, or about 2 months or longer as described herein. In particular embodiments, the non-treatment interval between two treatment courses is about 5 days, about 1 week, about 2 weeks, about 3 weeks, about 1 month, about 6 weeks, about 2 months (8 weeks), about 3 months, about 4 months, about 5 months, about 6 months, about 7 months, about 8 months, about 9 months, about 10 months, about 11 months, about 12 months (about 1 year), about 18 months (about 1.5 years), or longer. In certain specific embodiments, the non-treatment interval is about 2 years or about 3 years. In certain specific embodiments, the non-treatment time interval is at least about 14 days, at least about 21 days, at least about 1 month, at least about 2 months, at least about 3 months, at least about 4 months, at least about 5 months, at least about 6 months, or at least about 1 year. In certain embodiments, a course of treatment (whether daily, every other day, every 3^{rd} day, or other interval between administrations within the treatment course as described above (e.g., 1-14 days, 2-14 days, 2-21 days, or 1-21 days)) is administered about every 14 days (i.e., about every 2 weeks) (i.e., 14 days without compound selected from the table 1 ab and Table 1c treatment), about every 21 days (i.e., about every 3 weeks), about every 28 days (i.e., about every 4 weeks), about every one month, about every 36 days, about every 42 days, about every 54 days, about every 60 days, or about every month (about every 30 days), about every two months (about every 60 days), about every quarter (about every 90 days), or about semi-annually (about every 180 days). In other certain embodiments, a course of treatments (e.g., byway of non-limiting example, administration on at least one day or on at least two days during a course for about 2-21 days, about 2-14, days, about 5-14 days, about 7-14 days, about 9-14 days, about 5-21 days, about 7-21 days, about 9-21 days) is administered every 28 days, every 36 days, every 42 days, every 54 days, every 60 days, or every month (about every 30 days), every two months (about every 60 days), every quarter (about every 90 days), or semi-annually (about every 180 days), or about every year (about 12 months). In other embodiments, a course of treatment (such as by way of non-limiting examples, e.g., for about 5-28 days, about 7-28 days, or about 9-28 days whether daily, every other day, every 3^{rd} day, or other interval between administrations within the treatment course) is administered every 36 days, 42 days, 54 days, 60 days, or every month (about every 30 days), every two months (about every 60 days), every quarter (about every 90 days), or semi-annually (about every 180 days). In other particular embodiments, a course of treatment (e.g., for about 5-36 days, 7-36 days, or 9-36 days whether daily, every other day, every 3^{rd} day, or other interval between administrations within the treatment course) is administered every 42 days, 54 days, 60 days, or every month (about every 30 days), every two months (about every 60 days), every quarter (about every 90 days), or semi-annually (about every 180 days), or about every year (about 12 months).

In a particular embodiment, the treatment course is one day and the non-treatment interval is at least about 14 days, about 21 days, about 1 month, about 2 months (8 weeks), about 3 months, about 4 months, about 5 months, about 6 months, about 7 months, about 8 months, about 9 months, about 10 months, about 11 months, about 12 months (about 1 year), about 18 months (about 1.5 years), or longer. In other certain embodiments, the treatment course is at least two days or is at least 3 days and no longer than 10 days, and the non-treatment interval is at least about 14 days, about 21 days, about 1 month, about 2 months (8 weeks), about 3 months, about 4 months, about 5 months, about 6 months, about 7 months, about 8 months, about 9 months, about 10 months, about 11 months, about 12 months (about 1 year), about 18 months (about 1.5 years), or longer. In still another embodiment, the treatment course is at least three days and no longer than 10 days, no longer than 14 days, or no longer than 21 days, and the non-treatment interval is at least about 14 days, about 21 days, about 1 month, about 2 months (8 weeks), about 3 months, about 4 months, about 5 months, about 6 months, about 7 months, about 8 months, about 9 months, about 10 months, about 11 months, about 12 months (about 1 year), about 18 months (about 1.5 years), or longer. In still another embodiment, a treatment course (e.g., for about 5-42, 7-42, or 9-42 days whether daily, every other day, every 3^{rd} day, or other interval between administrations within the treatment course) is administered every 42 days, 60 days, or every month (about every 30 days), every two months (about every 60 days), every quarter (about every 90 days), or semi-annually (about every 180 days), or about every year (about 12 months). In a particular embodiment, the compound selected from the table 1 ab and Table 1c is administered daily for 5-14 days every 14 days (about every 2 weeks), or every 21-42 days. In another particular embodiment, the compound selected from the table 1 ab and Table 1c is administered daily for 5-14 days quarterly. In another particular embodiment, the compound selected from the table 1 ab and Table 1c is administered daily for 7-14 days every 21-42 days. In another particular embodiment, the compound selected from the table 1 ab and Table 1c is administered daily for 7.14 days quarterly. In still other particular embodiments, the compound selected from the table 1 ab and Table 1c is administered daily for 9-14 days every 21-42 days or every 9-14 days quarterly. In still other embodiments, the non-treatment interval may vary between treatment courses. By way of non-limiting example, the non-treatment interval may be 14 days after the first course of treatment and may be 21 days or longer after the second, third, or fourth (or more) course of treatment. In other particular embodiments, the compound selected from the table 1 ab and Table 1c is administered to the subject in need thereof once every 0.5-12 months. In other certain embodiments, the compound selected from the table 1 ab and Table 1c is administered to the subject in need once every 4-12 months.

In certain embodiments, a compound selected from the table 1 ab and Table 1c is administered to a subject to reduce the likelihood or the risk that the subject will develop a particular disorder or to delay onset of one or more symptoms of a senescence-associated disease or disorder. In certain embodiments, the compound selected from the table 1 ab and Table 1c is administered for one or more days (e.g., any number of consecutives days between and including 2-3, -4, -5, -6, -7, -8, -9, -10, -11, -12, -13, -14, -15, -16, -17, -18, -19, -20, and 2-21 days) every 3, 4, 5, 6, 7, 8, 9, 10, 11, or 12 months. In a particular embodiment, the compound selected from the table 1 ab and Table 1c is administered for one or more days (e.g., any number of consecutives days between and including 1-9 days) every 5 or 6 months.

Without wishing to be bound by any particular theory, in some embodiments, periodic administration of the compound selected from the table 1 ab and Table 1c kills newly formed senescent cells and thereby reduces (decreases, diminishes) the total number of senescent cells accumulating in the subject. In another embodiment, the total number of senescent cells accumulating in the subject is decreased or inhibited by administering the compound selected from the table 1 ab and Table 1c once or twice weekly or according to any of the other treatment courses described above. The total daily dose of a compound selected from the table 1 ab and Table 1c may be delivered as a single dose or as multiple doses on each day of administration. In other certain particular embodiments, when multiple cycles of the compound selected from the table 1 ab and Table 1c are administered, the dose of a compound selected from the table 1 ab and Table 1c administered on a single day may be less than the daily dose administered if only a single treatment course is intended to be administered.

In certain embodiments, method for treating a senescence-associated disease or disorder comprising administering to a subject in need thereof a small molecule compound selected from the table 1 ab and Table 1c that selectively kills senescent cells; wherein the senescence-associated disease of disorder is not a cancer, and wherein the compound selected from the table 1 ab and Table 1c is administered within one or two treatment cycles, typically two treatment cycles. In certain specific embodiments, the non-treatment interval is at least 2 weeks and each treatment course is no longer than 3 months.

Also provided herein are methods for selectively killing a senescent cell comprising contacting the senescent cell with a compound selected from the table 1 ab and Table 1c described herein (i.e., facilitating interaction or in some manner allowing the senescent cell and compound selected from the table 1 ab and Table 1c to interact) under conditions and for a time sufficient to kill the senescent cell. In such embodiments, the agent selectively kills senescent cells over non-senescent cells (i.e., the agent selectively kills senescent cells compared with killing of non-senescent cells), In certain embodiments, the senescent cell to be killed is present in a subject (e.g., a human or non-human animal). The Compound selected from the table 1 ab and Table 1c(s) may be administered to the subject according to the treatment cycles, treatment courses, and non-treatment intervals described above and herein.

In particular embodiments, a single (i.e., only, sole) compound selected from the table 1 ab and Table 1c is administered to the subject for treating a senescence-associated disease or disorder. In certain embodiments, administration of a single compound selected from the table 1 ab and Table 1c may be sufficient and clinically beneficial to treat a senescence-associated disease or disorder. Accordingly, in certain particular embodiments, a compound selected from the table 1 ab and Table 1c is administered as a monotherapy and is the single (i.e., only, sole) active agent administered to the subject for treating the condition or disease. Medications that are not necessarily excluded from administration to the subject when a compound selected from the table 1 ab and Table 1c is administered as a monotherapy include, by way of non-limiting examples, medications for other purposes such as palliative care or comfort (e.g., aspirin, acetominophen, ibuprofen, or prescription pain-killers; anti-itching topical medications) or for treating a different disease or condition, especially if the other medications are not senolytic agents, such as drugs for lowering cholesterol, statins, eye wetting agents, and other such medications familiar to a person skilled in the medical art.

By way of example, in certain embodiments, when the compound selected from the table 1 ab and Table 1c is an agent that can be cytotoxic to cancer cells and may be used in the oncology art in a manner for treating a cancer, the methods for treating a senescence associated disease or disorder comprise administering the compound selected from the table 1 ab and Table 1c in one or two or more treatment cycles, and the total dose of the compound selected from the table 1 ab and Table 1c administered during each treatment course, each treatment cycle, and/or cumulatively over two or more treatment cycles is an amount less than the amount effective for a cancer treatment. The amount of such a compound selected from the table 1 ab and Table 1c administered to a subject over a given time period (such as one week, two weeks, one month, six months, one year) for treating a senescence associated disease or disorder, for example, may be about from a 20-fold decrease to about a 5000-fold decrease in total amount compared with the total amount of the same agent administered to a subject who is receiving the agent for treatment of a cancer. The fold decrease in the amount (i.e., lesser amount) of the compound selected from the table 1 ab and Table 1c administered over a given time period (i.e., number of days, months, years) for treating a senescence associated disease or disorder may be about a 20-fold decrease, about a 25-fold decrease, about a 30-fold decrease, about a 40-fold decrease, about a 50-fold decrease, about a 60-fold decrease, about a 75-fold decrease, about a 100-fold decrease, about a 125-fold decrease, about a 150-fold decrease, about a 175-fold decrease, about a 200-fold decrease, about a 300-fold decrease, about a 400-fold decrease, about a 500-fold decrease, about a 750-fold decrease, about a 1000-fold decrease, about a 1250-fold decrease, about a 1500-fold decrease, about a 1750-fold decrease, about a 2000-fold decrease, about a 2250-fold decrease, about a 2500-fold decrease, about a 2750-fold decrease, about a 3000-fold decrease, about a 3250-fold decrease, about a 3500-fold decrease, about a 3750-fold decrease, about a 3000-fold decrease, about a 3500-fold decrease, about a 4000-fold decrease, about a 4500-fold decrease, or about a 5000-fold decrease compared with the amount of the agent administered to a subject for treating a cancer over the same length of time. A lower dose required for treating a senescence associated disease may also be attributable to the route of administration. For example, when a compound selected from the table 1 ab and Table 1c is used for treating a senescence-associated pulmonary disease or disorder (e.g., COPD, IPF), the compound selected from the table 1 ab and Table 1c may be delivered directly to the lungs (e.g., by inhalation, by intubation, intranasally, or intratracheally), and a lower dose per day and/or per treatment course is required than if the agent were administered orally. Also, by way of another example, when a compound selected from the table 1 ab and Table 1c is used for treating osteoarthritis or a senescence-associated dermatological disease or disorder, the compound selected from the table 1 ab and Table 1c may be delivered directly to the osteoarthritic joint (e.g., intra-articularly, intradermally, topically, transdermally) or to the skin (e.g., topically, subcutaneously, intradermally, transdermally), respectively, at a lower does per day and/or per treatment course than if the compound selected from the table 1 ab and Table 1c were administered orally. When a compound selected from the table 1 ab and Table 1c is delivered orally, for example, the dose of the compound selected from the table 1 ab and Table 1c per day may be the same amount as administered to a patient for treating a cancer; however, the amount of the agent that is delivered over a treatment course or treatment cycle is significantly less than the amount administered to a subject who receives the appropriate amount of the agent for treating a cancer.

In certain embodiments, the methods described herein comprise using the compound selected from the table 1 ab and Table 1c in an amount that is a reduced amount compared with the amount that may be delivered systemically, for example, orally or intravenously to a subject who receives the compound selected from the table 1 ab and Table 1c when the agent is used for treating a cancer. In certain specific embodiments, methods of treating a senescence-associated disease or disorder by selectively killing senescent cells comprises administering the compound selected from the table 1 ab and Table 1c at a dose that is at least 10% (i.e., one-tenth), at least 20% (one-fifth), 25% (one-fourth), 30%-33% (about one-third), 40% (two-fifths), or at least 50% (half) of the dose that is administered to a subject who has cancer for killing cancer cells during a treatment course, a treatment cycle, or two or more treatment cycles that form the cancer therapy protocol (i.e., regimen). In other particular embodiments, the dose of the Compound selected from the table 1 ab and Table 1c(s) used in the methods described herein is at least 60%, 70%, 80%, 85%, 90%, or 95% of the dose that is administered to a subject who has cancer. The therapeutic regimen, comprising the dose of compound selected from the table 1 ab and Table 1c and schedule and manner of administration that may be used for treating a senescence-associated disorder or disease is also a regimen insufficient to be significantly cytotoxic to non-senescent cells.

In other particular embodiments, the dose of the Compound selected from the table 1 ab and Table 1c used in the methods described herein is at least 1%, 5%, 10%, 20%, 30%, 40%, 50%, 60%, 70%, 80%, 85%, 90%, 95%, 115%, 115%, 130%, 150%, 175%, 200%, 250%, 300%, 350%, 400%, 450%, 500%, 550%, 600%, 650%, 700%, 750%, 800%, 850%, 900%, 950%, or 1000% of the lowest dose that is administered to a subject in Phase 1 clinical trial and in formulation, root of administration and regimen as it were in such Phase 1 clinical trial.

In other particular embodiments, the dose of the Compound selected from the table 1 ab and Table 1c used in the methods described herein is at least 1%, 5%, 10%, 20%, 30%, 40%, 50%, 60%, 70%, 80%, 85%, 90%, 95%, 115%, 115%, 130%, 150%, 175%, 200%, 250%, 300%, 350%, 400%, 450%, 500%, 550%, 600%, 650%, 700%, 750%, 800%, 850%, 900%, 950%, or 1000% of the highest dose that is administered to a subject in Phase 2 clinical trial and in formulation, root of administration and regimen as it were in such Phase 2 clinical trial for any one of the others indications

In other particular embodiments, the dose of the Compound selected from the table 1 ab and Table 1c used in the methods described herein is at least 1%, 5%, 10%, 20%, 30%, 40%, 50%, 60%, 70%, 80%, 85%, 90%, 95%, 115%, 115%, 130%, 150%, 175%, 200%, 250%, 300%, 350%, 400%, 450%, 500%, 550%, 600%, 650%, 700%, 750%, 800%, 850%, 900%, 950%, or 1000% of the highest dose that is administered to a subject in Phase 3 clinical trial and in formulation, root of administration and regimen as it were in such Phase 3 clinical trial for any one of the others indications.

In other particular embodiments, the dose of the Compound selected from the table 1 ab and Table 1c used in the methods described herein is at least 1%, 5%, 10%, 20%, 30%, 40%, 50%, 60%, 70%, 80%, 85%, 90%, 95%, 115%, 115%, 130%, 150%, 175%, 200%, 250%, 300%, 350%, 400%, 450%, 500%, 550%, 600%, 650%, 700%, 750%, 800%, 850%, 900%, 950%, or 1000% of the highest dose that is approved and in formulation, root of administration and regimen as it were approved for other any one of others indications.

In certain embodiments, a method for treating a senescence-associated disease or disorder that is not a cancer comprises administering to a subject in need thereof a therapeutically effective amount of a small molecule compound selected from the table 1 ab and Table 1c that selectively kills senescent cells (i.e., selectively kills senescent cells over non-senescent cells or compared with non-senescent cells) and which agent is cytotoxic to cancer cells, wherein the compound selected from the table 1 ab and Table 1c is administered within at least one treatment cycle, which treatment cycle comprises a treatment course followed by a non-treatment interval. The total dose of the compound selected from the table 1 ab and Table 1c administered during the treatment course, and/or the total dose of the compound selected from the table 1 ab and Table 1c administered during the treatment cycle, and/or the total dose of the compound selected from the table 1 ab and Table 1c administered during two or more treatment cycles is an amount less than the amount effective for a cancer treatment. In other certain embodiments, the compound selected from the table 1 ab and Table 1c is administered as a monotherapy, and is the single active compound selected from the table 1 ab and Table 1c administered to the subject for treating the disease or disorder. The number of days in the treatment course and the treatment interval are described in detail herein.

In one embodiment, a method is provided herein for treating a senescence-associated disease or disorder, wherein the senescence-associated disease is not cancer and the method comprises administering to a subject in need thereof a compound selected from the table 1 ab and Table 1c or small molecule senolytic compound that selectively kills senescent cells, and the administration is for a short duration (e.g., shorter than may be used for a particular agent for treating a cancer), such as a single day, 2 days, 3 days, 4 days, 5 days, 6 days, 7 days, 8 days, 9 days, 10 days, 11 days, 12 days, 13 days, 14 days, or 15 days. In these particular embodiments, this treatment course on any number of days between 1-15 days is a single treatment course and is not repeated. In another particular embodiment, a compound selected from the table 1 ab and Table 1c is administered for 16 days, 17 days, 18 days, 19 days, 20 days, 21 days, 22 days, 23 days, 24 days, 25 days, 26 days, 27 days, 28 days, 29 days, 30 days, or 31 days as a single treatment course that is not repeated.

### Senescence-Associated Diseases and Disorders

Methods are provided herein for treating conditions, diseases, or disorders related to, associated with, or caused by cellular senescence, including age-related diseases and disorders in a subject in need thereof. A senescence-associated disease or disorder may also be called herein a senescent cell-associated disease or disorder. Senescence-associated diseases and disorders include, for example, cardiovascular diseases and disorders, inflammatory diseases and disorders, autoimmune diseases and disorders, pulmonary diseases and disorders, eye diseases and disorders, metabolic diseases and disorders, neurological diseases and disorders (e.g., neurodegenerative diseases and disorders); age-related diseases and disorders induced by senescence; skin conditions; age-related diseases; dermatological diseases and disorders; and transplant related diseases and disorders. A prominent feature of aging is a gradual loss of function, or degeneration that occurs at the molecular, cellular, tissue, and organismal levels. Methods are provided herein for treatment of this gradual loss and degeneration by administering a compound selected from the table 1 ab and Table 1c. Age-related degeneration gives rise to well-recognized pathologies, such as sarcopenia, atherosclerosis and heart failure, osteoporosis, pulmonary insufficiency, renal failure, neurodegeneration (including macular degeneration, Alzheimer's disease, and Parkinson's disease), and many others. Although different mammalian species vary in their susceptibilities to specific age-related pathologies, collectively, age-related pathologies generally rise with approximately exponential kinetics beginning at about the mid-point of the species-specific life span (e.g., 50-60 years of age for humans). Methods are provided herein for reducing these susceptibilities by administering a compound selected from the table 1 ab and Table 1c.

Examples of senescence-associated conditions, disorders, or diseases that may be treated by administering any one of the Compounds selected from the table 1 ab and Table 1c described herein according to the methods described herein include, cognitive diseases (e.g., mild cognitive impairment (MCI), Alzheimer's disease and other dementias; Huntington's disease); cardiovascular disease (e.g., atherosclerosis, cardiac diastolic dysfunction, aortic aneurysm, angina, arrhythmia, cardiomyopathy, congestive heart failure, coronary artery disease, myocardial infarction, endocarditis, hypertension, carotid artery disease, peripheral vascular diseases, cardiac stress resistance, cardiac fibrosis); metabolic diseases and disorders (e.g., obesity, diabetes, metabolic syndrome); motor function diseases and disorders (e.g., Parkinson's disease, motor neuron dysfunction (MND); Huntington's disease); cerebrovascular disease; emphysema; osteoarthritis; benign prostatic hypertrophy; pulmonary diseases (e.g., idiopathic pulmonary fibrosis, chronic obstructive pulmonary disease (COPD), emphysema, obstructive bronchiolitis, asthma); inflammatory/autoimmune diseases and disorders (e.g., osteoarthritis, eczema, psoriasis, osteoporosis, mucositis, transplantation related diseases and disorders); ophthalmic diseases or disorders (e.g., age-related macular degeneration, cataracts, glaucoma, vision loss, presbyopia); diabetic ulcer; metastasis; a chemotherapeutic side effect, a radiotherapy side effect; aging-related diseases and disorders (e.g., kyphosis, renal dysfunction, frailty, hair loss, hearing loss, muscle fatigue, skin conditions, sarcopenia, and herniated intervertebral disc) and other age-related diseases that are induced by senescence (e.g., diseases/disorders resulting from irradiation, chemotherapy, smoking tobacco, eating a high fat/high sugar diet, and environmental factors); wound healing; skin nevi; fibrotic diseases and disorders (e.g., cystic fibrosis, renal fibrosis, liver fibrosis, pulmonary fibrosis, oral submucous fibrosis, cardiac fibrosis, and pancreatic fibrosis). In certain embodiments, any one or more of the diseases or disorders described above or herein may be excluded.

In a more specific embodiment, methods are provided for treating a senescence-associated disease or disorder by killing senescent cells (i.e., established senescent cells) associated with the disease or disorder in a subject who has the disease or disorder by administering a Compound selected from the table 1 ab and Table 1c, wherein the disease or disorder is osteoarthritis; idiopathic pulmonary fibrosis; chronic obstructive pulmonary disease (COPD); or atherosclerosis.

Subjects (i.e., patients, individuals (human or non-human animals)) who may benefit from use of the methods described herein that comprise administering a compound selected from the table 1 ab and Table 1c include those who may also have a cancer. The subject treated by these methods may be considered to be in partial or complete remission (also called cancer remission). As discussed in detail herein, the Compounds selected from the table 1 ab and Table 1c for use in methods for selective killing of senescent cells are not intended to be used as a treatment for cancer, that is, in a manner that kills or destroys the cancer cells in a statistically significant manner. Therefore, in some embodiments, the methods disclosed herein do not encompass use of the Compounds selected from the table 1 ab and Table 1c in a manner that would be considered a primary therapy for the treatment of a cancer. Even though a Compound selected from the table 1 ab and Table 1c, alone or with other chemotherapeutic or radiotherapy agents, are not used in a manner that is sufficient to be considered as a primary cancer therapy, the methods and Compounds selected from the table 1 ab and Table 1c described herein may be used in a manner (e.g., a short term course of therapy) that is useful for inhibiting metastases. In other certain embodiments, the subject to be treated with the compound selected from the table 1 ab and Table 1c does not have a cancer (i.e., the subject has not been diagnosed as having a cancer by a person skilled in the medical art).

### Cardiovascular Diseases and Disorders.

In another embodiment, the senescence-associated disease or disorder treated by the methods, comprising administering a compound selected from the table 1 ab and Table 1c, described herein is a cardiovascular disease. The cardiovascular disease may be any one or more of angina, arrhythmia, atherosclerosis, cardiomyopathy, congestive heart failure, coronary artery disease (CAD), carotid artery disease, endocarditis, heart attack (coronary thrombosis, myocardial infarction [MI]), high blood pressure/hypertension, aortic aneurysm, brain aneurysm, cardiac fibrosis, cardiac diastolic dysfunction, hypercholesterolemia/hyperlipidemia, mitral valve prolapse, peripheral vascular disease (e.g., peripheral artery disease (PAD)), cardiac stress resistance, and stroke.

In certain embodiments, methods are provided for treating senescence-associated cardiovascular disease that is associated with or caused by arteriosclerosis (i.e., hardening of the arteries). In certain embodiments, such methods comprise administering a compound selected from the table 1 ab and Table 1c. The cardiovascular disease may be any one or more of atherosclerosis (e.g., coronary artery disease (CAD) and carotid artery disease); angina, congestive heart failure, and peripheral vascular disease (e.g., peripheral artery disease (PAD)), wherein in some embodiments such methods comprise administering a compound selected from the table 1 ab and Table 1c. The methods for treating a cardiovascular disease that is associated with or caused by arteriosclerosis may reduce the likelihood of occurrence of high blood pressure/hypertension, angina, stroke, and heart attack (i.e., coronary thrombosis, myocardial infarction (MI)). In certain embodiments, methods are provided for stabilizing atherosclerotic plaque(s) in a blood vessel (e.g., artery) of a subject, thereby reducing the likelihood of occurrence or delaying the occurrence of a thrombotic event, such as stroke or MI. In certain embodiments, these methods, some of them comprising administration of a compound selected from the table 1 ab and Table 1c reduce (i.e., cause decrease of) the lipid content of an atherosclerotic plaque in a blood vessel (e.g., artery) of the subject and/or increase the fibrous cap thickness (i.e., cause an increase, enhance or promote thickening of the fibrous cap).

In one embodiment, methods are provided for inhibiting the formation of atherosclerotic plaques (or reducing, diminishing, causing decrease in formation of atherosclerotic plaques) by administering a Compound selected from the table 1 ab and Table 1c. In other embodiments, methods are provided for reducing (decreasing, diminishing) the amount (i.e., level) of plaque. Reduction in the amount of plaque in a blood vessel (e.g., artery) may be determined, for example, by a decrease in surface area of the plaque, or by a decrease in the extent or degree (e.g., percent) of occlusion of a blood vessel (e.g., artery), which can be determined by angiography or other visualizing methods used in the cardiovascular art. Also provided herein are methods for increasing the stability (or improving, promoting, enhancing stability) of atherosclerotic plaques that are present in one or more blood vessels (e.g., one or more arteries) of a subject, which methods comprise administering to the subject any one of the Compounds selected from the table 1 ab and Table 1c described herein.

In some embodiment, this invention suggested a method of atheromatous plaque stabilization comprising administering to the subject any one of the Compounds selected from the table 1 ab and Table 1c.

Subjects suffering from cardiovascular disease can be identified using standard diagnostic methods known in the art for cardiovascular disease. Generally, diagnosis of atherosclerosis and other cardiovascular disease is based on symptoms (e.g., chest pain or pressure (angina), numbness or weakness in arms or legs, difficulty speaking or slurred speech, drooping muscles in face, leg pain, high blood pressure, kidney failure and/or erectile dysfunction), medical history, and/or physical examination of a patient. Diagnosis may be confirmed by angiography, ultrasonography, or other imaging tests. Subjects at risk of developing cardiovascular disease include those having any one or more of predisposing factors, such as a family history of cardiovascular disease and those having other risk factors (i.e., predisposing factors) such as high blood pressure, dyslipidemia, high cholesterol, diabetes, obesity and cigarette smoking, sedentary lifestyle, and hypertension. In a certain embodiment, the cardiovascular disease that is a senescence cell associated disease/disorder is atherosclerosis.

The effectiveness of one or more Compounds selected from the table 1 ab and Table 1c for treating or preventing (i.e., reducing or decreasing the likelihood of developing or occurrence of) a cardiovascular disease (e.g., atherosclerosis) can readily be determined by a person skilled in the medical and clinical arts. One or any combination of diagnostic methods, including physical examination, assessment and monitoring of clinical symptoms, and performance of analytical tests and methods described herein and practiced in the art (e.g., angiography, electrocardiography, stress test, non-stress test), may be used for monitoring the health status of the subject. The effects of the treatment of a compound selected from the table 1 ab and Table 1c or pharmaceutical composition comprising same can be analyzed using techniques known in the art, such as comparing symptoms of patients suffering from or at risk of cardiovascular disease that have received the treatment with those of patients without such a treatment or with placebo treatment.

### Inflammatory and Autoimmune Diseases and Disorders.

In certain embodiments, a senescence-associated disease or disorder is an inflammatory disease or disorder, such as by way of non-limiting example, osteoarthritis, that may be treated or prevented (i.e., likelihood of occurrence is reduced) according to the methods described herein that comprise administration of a Compound selected from the table 1 ab and Table 1c. Other inflammatory of autoimmune diseases or disorders that may be treated by administering a compound selected from the table 1 ab and Table 1c such as the inhibitors and antagonists described herein include osteoporosis, psoriasis, oral mucositis, rheumatoid arthritis, inflammatory bowel disease, eczema, kyphosis, herniated intervertebral disc, and the pulmonary diseases, COPD and idiopathic pulmonary fibrosis.

In certain embodiments, a senescence-associated disease or disorder is age- and high fat-diet-induced vascular calcification and hyporeactivity, and radiation-induced muscle dysfunction.

Chronic inflammation is thought to be the main age-related factor that contributes to osteoarthritis. In combination with aging, joint overuse and obesity appear to promote osteoarthritis.

In certain embodiments, a compound selected from the table 1 ab and Table 1c prevents (i.e., reduces the likelihood of occurrence), reduces or inhibits loss or erosion of proteoglycan layers in a joint, reduces inflammation in the affected joint, and promotes (i.e., stimulates, enhances, induces) production of collagen (e.g., type 2 collagen). In certain embodiments, administration of a compound selected from the table 1 ab and Table 1c causes a reduction in the amount (i.e., level) of inflammatory cytokines, such as IL-6, produced in a joint and inflammation is reduced. Methods are provided herein for treating osteoarthritis, and/or for selectively killing senescent cells in an osteoarthritic joint of a subject, and/or inducing collagen (such as Type 2 collagen) production in the joint of a subject in need thereof by administering at least one compound selected from the table 1 ab and Table 1c (which may be combined with at least one pharmaceutically acceptable excipient to form a pharmaceutical composition) to the subject. A compound selected from the table 1 ab and Table 1c also may be used for decreasing (inhibiting, reducing) production of metalloproteinase 13 (MMP-13), which degrades collagen in a joint, and for restoring proteoglycan layer or inhibiting loss and/or degradation of the proteoglycan layer. Treatment with the compound selected from the table 1 ab and Table 1c thereby also prevents (i.e., reduces likelihood of occurrence of), inhibits, or decreases erosion, or slows (i.e., decreases rate) erosion of the bone. As described in detail herein, in certain embodiments, the compound selected from the table 1 ab and Table 1c is administered directly to an osteoarthritic joint (e.g., by intra-articularly, topical, transdermal, intradermal, or subcutaneous delivery). Treatment with a compound selected from the table 1 ab and Table 1c can also restore, improve, or inhibit deterioration of strength of a joint. In addition, the methods comprising administering a compound selected from the table 1 ab and Table 1c can reduce joint pain and are therefore useful for pain management of osteoarthritic joints.

The effectiveness of one or more Compounds selected from the table 1 ab and Table 1c for treatment or prophylaxis of osteoarthritis in a subject and monitoring of a subject who receives one or more Compounds selected from the table 1 ab and Table 1c can readily be determined by a person skilled in the medical and clinical arts. One or any combination of diagnostic methods, including physical examination (such as determining tenderness, swelling or redness of the affected joint), assessment and monitoring of clinical symptoms (such as pain, stiffness, mobility), and performance of analytical tests and methods described herein and practiced in the art (e.g., determining the level of inflammatory cytokines or chemokines; X-ray images to determine loss of cartilage as shown by a narrowing of space between the bones in a joint; magnetic resonance imaging (MRI), providing detailed images of bone and soft tissues, including cartilage), may be used for monitoring the health status of the subject. The effects of the treatment of one or more Compounds selected from the table 1 ab and Table 1c can be analyzed by comparing symptoms of patients suffering from or at risk of an inflammatory disease or disorder, such as osteoarthritis, who have received the treatment with those of patients who have not received such a treatment or who have received a placebo treatment.

In certain embodiments, Compounds selected from the table 1 ab and Table 1c may be used for treating and/or preventing (i.e., decreasing or reducing the likelihood of occurrence) rheumatoid arthritis (RA). Dysregulation of innate and adaptive immune responses characterize rheumatoid arthritis (RA), which is an autoimmune disease the incidence of which increases with age. Rheumatoid arthritis is a chronic inflammatory disorder that typically affects the small joints in hands and feet. Whereas osteoarthritis results from, at least in part, wear and tear of a joint, rheumatoid arthritis affects the lining of joints, resulting in a painful swelling that can lead to bone erosion and joint deformity. RA can sometimes also affect other organs of the body, such as the skin, eyes, lungs and blood vessels. RA can occur in a subject at any age; however, RA usually begins to develop after age 40. The disorder is much more common in women. In certain embodiments of the methods described herein, RA is excluded.

Chronic inflammation may also contribute to other age-related or aging related diseases and disorders, such as kyphosis and osteoporosis. Kyphosis is a severe curvature in the spinal column, and it is frequently seen with normal and premature aging. Age-related kyphosis often occurs after osteoporosis weakens spinal bones to the point that they crack and compress. A few types of kyphosis target infants or teens. Severe kyphosis can affect lungs, nerves, and other tissues and organs, causing pain and other problems. Kyphosis has been associated with cellular senescence. Characterizing the capability of a compound selected from the table 1 ab and Table 1c for treating kyphosis may be determined in pre-clinical animal models used in the art. By way of example, TTD mice develop kyphosis (see, e.g., de Boer et al. (2002) Science 296: 1276-1279); other mice that may be used include BubR1^{H/H} mice, which are also known to develop kyphosis (see, e.g., Baker et al. (2011) Nature 479: 232-36). Kyphosis formation is visually measured over time. The level of senescent cells decreased by treatment with the compound selected from the table 1 ab and Table 1c can be determined by detecting the presence of one or more senescent cell associated markers such as by SA-β-GAL staining.

Osteoporosis is a progressive bone disease that is characterized by a decrease in bone mass and density that may lead to an increased risk of fracture. Bone mineral density (BMD) is reduced, bone microarchitecture deteriorates, and the amount and variety of proteins in bone are altered. Osteoporosis is typically diagnosed and monitored by a bone mineral density test. Post-menopausal women or women who have reduced estrogen are most at risk. While both men and women over 75 are at risk, women are twice as likely to develop osteoporosis than men. The level of senescent cells decreased by treatment with the compound selected from the table 1 ab and Table 1c can be determined by detecting the presence of one or more senescent cell associated markers such as by SA-β-GAL staining.

In still other embodiments, an inflammatory/autoimmune disorder that may be treated or prevented (i.e., likelihood of occurrence is reduced) with the Compounds selected from the table 1 ab and Table 1c described herein includes irritable bowel syndrome (IBS) and inflammatory bowel diseases, such as ulcerative colitis and Crohn's disease. Inflammatory bowel disease (IBD) involves chronic inflammation of all or part of the digestive tract. In addition to life-threatening complications arising from IBD, the disease can be painful and debilitating. Ulcerative colitis is an inflammatory bowel disease that causes long-lasting inflammation in part of the digestive tract. Symptoms usually develop over time, rather than suddenly. Ulcerative colitis usually affects only the innermost lining of the large intestine (colon) and rectum. Crohn's disease is an inflammatory bowel disease that causes inflammation anywhere along the lining of your digestive tract, and often extends deep into affected tissues. This can lead to abdominal pain, severe diarrhea, and malnutrition. The inflammation caused by Crohn's disease can involve different areas of the digestive tract. Diagnosis and monitoring of the diseases is performed according to methods and diagnostic tests routinely practiced in the art, including blood tests, colonoscopy, flexible sigmoidoscopy, barium enema, CT scan, MRI, endoscopy, and small intestine imaging.

In other embodiments, the methods described herein may be useful for treating a subject who has herniated intervertebral discs. In some embodiments, such methods comprise administering Compounds selected from the table 1 ab and Table 1c. Subjects with these herniated discs exhibit elevated presence of cell senescence in the blood and in vessel walls. Symptoms of a herniated intervertebral disc may include pain, numbness or tingling, or weakness in an arm or leg. Increased levels of proinflammatory molecules and matrix metalloproteases are also found in aging and degenerating discs tissues, suggesting a role for senescence cells. Animal models may be used to characterize the effectiveness of a compound selected from the table 1 ab and Table 1c in treating herniated intervertebral discs; degeneration of the intervertebral disc is induced in mice by compression and disc strength evaluated (see e.g., Lotz et al. (1998) Spine (Philadelphia Pa. 1976). 23:2493-506).

Other inflammatory or autoimmune diseases that may be treated or prevented (i.e., likelihood of occurrence is reduced) by using a compound selected from the table 1 ab and Table 1c include eczema, psoriasis, osteoporosis, and pulmonary diseases (e.g., chronic obstructive pulmonary disease (COPD), idiopathic pulmonary fibrosis (IPF), asthma), inflammatory bowel disease, and mucositis (including oral mucositis, which in some instances is induced by radiation). Certain fibrosis or fibrotic conditions of organs such as renal fibrosis, liver fibrosis, pancreatic fibrosis, cardiac fibrosis, skin wound healing, and oral submucous fibrosis may be treated with using the Compound selected from the table 1 ab and Table 1c.

In certain embodiments, the senescent cell associated disorder is an inflammatory disorder of the skin, such as by way of a non-limiting examples, psoriasis and eczema that may be treated or prevented (i.e., likelihood of occurrence is reduced) according to the methods described herein that comprise administration of a Compound selected from the table 1 ab and Table 1c. Psoriasis is characterized by abnormally excessive and rapid growth of the epidermal layer of the skin. A diagnosis of psoriasis is usually based on the appearance of the skin. Skin characteristics typical for psoriasis are scaly red plaques, papules, or patches of skin that may be painful and itch. In psoriasis, cutaneous and systemic overexpression of various proinflammatory cytokines is observed such as IL-6, a key component of the SASP. Eczema is an inflammation of the skin that is characterized by redness, skin swelling, itching and dryness, crusting, flaking, blistering, cracking, oozing, or bleeding. The effectiveness of Compounds selected from the table 1 ab and Table 1c for treatment of psoriasis and eczema and monitoring of a subject who receives such a compound selected from the table 1 ab and Table 1c can be readily determined by a person skilled in the medical or clinical arts. One or any combination of diagnostic methods, including physical examination (such as skin appearance), assessment of monitoring of clinical symptoms (such as itching, swelling, and pain), and performance of analytical tests and methods described herein and practiced in the art (i.e., determining the level of pro-inflammatory cytokines). In some embodiments, Compound selected from the table 1 ab and Table 1c is effective in alleviating of at least one symptoms of at least one inflammatory disorder.

Other immune disorders or conditions that may be treated or prevented (i.e., likelihood of occurrence is reduced) with a compound selected from the table 1 ab and Table 1c include conditions resulting from a host immune response to an organ transplant (e.g., kidney, bone marrow, liver, lung, or heart transplant), such as rejection of the transplanted organ. The compound selected from the table 1 ab and Table 1c may be used for treating or reducing the likelihood of occurrence of graft-vs-host disease.

### Pulmonary Diseases and Disorders.

In one embodiment, methods are provided for treating ore preventing (i.e., reducing the likelihood of occurrence of) a senescence-associated disease or disorder that is a pulmonary disease or disorder by killing senescent cells (i.e., established senescent cells) associated with the disease or disorder in a subject who has the disease or disorder by administering a Compound selected from the table 1 ab and Table 1c. Senescence associated pulmonary diseases and disorders include, for example, idiopathic pulmonary fibrosis (IPF), chronic obstructive pulmonary disease (COPD), asthma, cystic fibrosis, bronchiectasis, and emphysema. In some embodiments, such methods comprise administration of a Compound selected from the table 1 ab and Table 1c.

COPD is a lung disease defined by persistently poor airflow resulting from the breakdown of lung tissue (emphysema) and the dysfunction of the small airways (obstructive bronchiolitis). Primary symptoms of COPD include shortness of breath, wheezing, chest tightness, chronic cough, and excess sputum production. Elastase from cigarette smoke-activated neutrophils and macrophages disintegrates the extracellular matrix of alveolar structures, resulting in enlarged air spaces and loss of respiratory capacity. COPD is most commonly caused by tobacco smoke (including cigarette smoke, cigar smoke, secondhand smoke, pipe smoke), occupational exposure (e.g., exposure to dust, smoke or fumes), and pollution, occurring over decades thereby implicating aging as a risk factor for developing COPD. In some embodiments, Compound selected from the table 1 ab and Table 1c is effective in alleviating of at least one symptoms of COPD.

Pulmonary fibrosis is a chronic and progressive lung disease characterized by stiffening and scarring of the lung, which may lead to respiratory failure, lung cancer, and heart failure. Fibrosis is associated with repair of epithelium. Fibroblasts are activated, production of extracellular matrix proteins is increased, and transdifferentiation to contractile myofibroblasts contribute to wound contraction. A provisional matrix plugs the injured epithelium and provides a scaffold for epithelial cell migration, involving an epithelial-mesenchymal transition (EMT). Blood loss associated with epithelial injury induces platelet activation, production of growth factors, and an acute inflammatory response. Normally, the epithelial barrier heals and the inflammatory response resolves. However, in fibrotic disease the fibroblast response continues, resulting in unresolved wound healing. Formation of fibroblastic foci is a feature of the disease, reflecting locations of ongoing fibrogenesis. As the name connotes, the etiology of IPF is unknown. The involvement of cellular senescence in IPF is suggested by the observations that the incidence of the disease increases with age and that lung tissue in IPF patients is enriched for SA-β-Gal-positive cells and contains elevated levels of the senescence marker p21. Without wishing to be bound by theory, the contribution of cellular senescence to IPF is suggested by the report that SASP components of senescent cells, such as IL-6, IL-8, and IL-1β, promote fibroblast-to-myofibroblast differentiation and epithelial-mesenchymal transition, resulting in extensive remodeling of the extracellular matrix of the alveolar and interstitial spaces (see, e.g., Minagawa et al., supra).

Subjects at risk of developing pulmonary fibrosis include those exposed to environmental or occupational pollutants, such as asbestosis and silicosis; who smoke cigarettes; having some typical connective tissue diseases such as rheumatoid arthritis, SLE and scleroderma; having other diseases that involve connective tissue, such as sarcoidosis and Wegener's granulomatosis; having infections; taking certain medications (e.g., amiodarone, bleomycin, busufan, methotrexate, and nitrofurantoin); those subject to radiation therapy to the chest; and those whose family member has pulmonary fibrosis.

Symptoms of COPD may include any one of shortness of breath, especially during physical activities; wheezing; chest tightness; having to clear your throat first thing in the morning because of excess mucus in the lungs; a chronic cough that produces sputum that may be clear, white, yellow or greenish; blueness of the lips or fingernail beds (cyanosis); frequent respiratory infections; lack of energy; unintended weight loss (observed in later stages of disease). Subjects with COPD may also experience exacerbations, during which symptoms worsen and persist for days or longer. Symptoms of pulmonary fibrosis are known in the art and include shortness of breath, particularly during exercise; dry, hacking cough; fast, shallow breathing; gradual unintended weight loss; tiredness; aching joints and muscles; and clubbing (widening and rounding of the tips of the fingers or toes).

Subjects suffering from COPD or pulmonary fibrosis can be identified using standard diagnostic methods routinely practiced in the art. Monitoring the effect of one or more Compounds selected from the table 1 ab and Table 1c administered to a subject who has or who is at risk of developing a pulmonary disease may be performed using the methods typically used for diagnosis. Generally, one or more of the following exams or tests may be performed: physical exam, patient's medical history, patient's family's medical history, chest X-ray, lung function tests (such as spirometry), blood test (e.g., arterial blood gas analysis), bronchoalveolar lavage, lung biopsy, CT scan, and exercise testing.

Other pulmonary diseases or disorders that may be treated by using a compound selected from the table 1 ab and Table 1c include, for example, emphysema, asthma, bronchiectasis, and cystic fibrosis. These diseases may also be exacerbated by tobacco smoke (including cigarette smoke, cigar smoke, secondhand smoke, pipe smoke), occupational exposure (e.g., exposure to dust, smoke or fumes), infection, and/or pollutants that induce cells into senescence and thereby contribute to inflammation. Emphysema is sometimes considered as a subgroup of COPD.

Bronchiectasis is results from damage to the airways that causes them to widen and become flabby and scarred. Bronchiectasis usually is caused by a medical condition that injures the airway walls or inhibits the airways from clearing mucus. Examples of such conditions include cystic fibrosis and primary ciliary dyskinesia (PCD). When only one part of the lung is affected, the disorder may be caused by a blockage rather than a medical condition.

The methods described herein for treating or preventing (i.e., reducing the likelihood of occurrence of) a senescence associate pulmonary disease or disorder may also be used for treating a subject who is aging and has loss (or degeneration) of pulmonary function (i.e., declining or impaired pulmonary function compared with a younger subject) and/or degeneration of pulmonary tissue. The respiratory system undergoes various anatomical, physiological and immunological changes with age. The structural changes include chest wall and thoracic spine deformities that can impair the total respiratory system compliance resulting in increased effort to breathe. The respiratory system undergoes structural, physiological, and immunological changes with age. An increased proportion of neutrophils and lower percentage of macrophages can be found in bronchoalveolar lavage (BAL) of older adults compared with younger adults. Persistent low grade inflammation in the lower respiratory tract can cause proteolytic and oxidant-mediated injury to the lung matrix resulting in loss of alveolar unit and impaired gas exchange across the alveolar membrane seen with aging. Sustained inflammation of the lower respiratory tract may predispose older adults to increased susceptibility to toxic environmental exposure and accelerated lung function decline. Oxidative stress exacerbates inflammation during aging. Alterations in redox balance and increased oxidative stress during aging precipitate the expression of cytokines, chemokines, and adhesion molecules, and enzymes. Constitutive activation and recruitment of macrophages, T cells, and mast cells foster release of proteases leading to extracellular matrix degradation, cell death, remodeling, and other events that can cause tissue and organ damage during chronic inflammation. By administering a compound selected from the table 1 ab and Table 1c to an aging subject (which includes a middle-aged adult who is asymptomatic), the decline in pulmonary function may be decelerated or inhibited by killing and removing senescent cells from the respiratory tract.

The effectiveness of a compound selected from the table 1 ab and Table 1c can readily be determined by a person skilled in the medical and clinical arts. One or any combination of diagnostic methods, including physical examination, assessment and monitoring of clinical symptoms, and performance of analytical tests and methods described herein, may be used for monitoring the health status of the subject. The effects of the treatment of a compound selected from the table 1 ab and Table 1c or pharmaceutical composition comprising the agent can be analyzed using techniques known in the art, such as comparing symptoms of patients suffering from or at risk of the pulmonary disease that have received the treatment with those of patients without such a treatment or with placebo treatment. In addition, methods and techniques that evaluate mechanical functioning of the lung, for example, techniques that measure lung capacitance, elastance, and airway hypersensitivity may be performed. To determine lung function and to monitor lung function throughout treatment, any one of numerous measurements may be obtained, expiratory reserve volume (ERV), forced vital capacity (FVC), forced expiratory volume (FEV) (e.g., FEV in one second, FEV1), FEV1/FBV ratio, forced expiratory flow 25% to 75%, and maximum voluntary ventilation (MVV), peak expiratory flow (PEF), slow vital capacity (SVC). Total lung volumes include total lung capacity (TLC), vital capacity (VC), residual volume (RV), and functional residual capacity (FRC). Gas exchange across alveolar capillary membrane can be measured using diffusion capacity for carbon monoxide (DLCO). Peripheral capillary oxygen saturation (SpO₂) can also be measured; normal oxygen levels are typically between 95% and 100%. An SpO₂ level below 90% suggests the subject has hypoxemia. Values below 80% are considered critical and requiring intervention to maintain brain and cardiac function and avoid cardiac or respiratory arrest.

### Neurological Diseases and Disorders.

Senescence-associated diseases or disorders treatable by administering a compound selected from the table 1 ab and Table 1c described herein include neurological diseases or disorders. Such senescence-associated diseases and disorders include Parkinson's disease, Alzheimer's disease (and other dementias), motor neuron dysfunction (MND), mild cognitive impairment (MCI), Huntington's disease, and diseases and disorders of the eyes, such as age-related macular degeneration. Other diseases of the eye that are associated with increasing age are glaucoma, vision loss, presbyopia, and cataracts.

Parkinson's disease (PD) is the second most common neurodegenerative disease. It is a disabling condition of the brain characterized by slowness of movement (bradykinesia), shaking, stiffness, and in the later stages, loss of balance. Many of these symptoms are due to the loss of certain nerves in the brain, which results in the lack of dopamine. This disease is characterized by neurodegeneration, such as the loss of about 50% to 70% of the dopaminergic neurons in the substantia nigra pars compacta, a profound loss of dopamine in the striatum, and/or the presence of intracytoplasmic inclusions (Lewy bodies), which are composed mainly of alpha-synuclein and ubiquitin. Parkinson's disease also features locomotor deficits, such as tremor, rigidity, bradykinesia, and/or postural instability. Subjects at risk of developing Parkinson's disease include those having a family history of Parkinson's disease and those exposed to pesticides (e.g., rotenone or paraquat), herbicides (e.g., agent orange), or heavy metals. Senescence of dopamine-producing neurons is thought to contribute to the observed cell death in PD through the production of reactive oxygen species; therefore, the methods and Compounds selected from the table 1 ab and Table 1c described herein are useful for treatment and prophylaxis of Parkinson's disease.

Methods for detecting, monitoring or quantifying neurodegenerative deficiencies and/or locomotor deficits associated with Parkinson's diseases are known in the art, such as histological studies, biochemical studies, and behavioral assessment (see, e.g., U.S. Application Publication No. 2012/0005765). Symptoms of Parkinson's disease are known in the art and include, but are not limited to, difficulty starting or finishing voluntary movements, jerky, stiff movements, muscle atrophy, shaking (tremors), and changes in heart rate, but normal reflexes, bradykinesia, and postural instability. There is a growing recognition that people diagnosed with Parkinson's disease may have cognitive impairment, including mild cognitive impairment, in addition to their physical symptoms.

Alzheimer's disease (AD) is a neurodegenerative disease that shows a slowly progressive mental deterioration with failure of memory, disorientation, and confusion, leading to profound dementia. Age is the single greatest predisposing risk factor for developing AD, which is the leading cause of dementia in the elderly. Early clinical symptoms show remarkable similarity to mild cognitive impairment (see below). As the disease progresses, impaired judgment, confusion, behavioral changes, disorientation, and difficulty in walking and swallowing occur.

Alzheimer's disease is characterized by the presence of neurofibrillary tangles and amyloid (senile) plaques in histological specimens. The disease predominantly involves the limbic and cortical regions of the brain. The argyrophilic plaques containing the amyloidogenic Aβ fragment of amyloid precursor protein (APP) are scattered throughout the cerebral cortex and hippocampus. Neurofibrillary tangles are found in pyramidal neurons predominantly located in the neocortex, hippocampus, and nucleus basalis of Meynert. Other changes, such as granulovacuolar degeneration in the pyramidal cells of the hippocampus, and neuron loss and gliosis in the cortex and hippocampus, are observed. Subjects at risk of developing Alzheimer's disease include those of advanced age, those with a family history of Alzheimer's disease, those with genetic risk genes (e.g., ApoE4) or deterministic gene mutations (e.g., APP, PS1, or PS2), and those with history of head trauma or heart/vascular conditions (e.g., high blood pressure, heart disease, stroke, diabetes, high cholesterol).

A number of behavioral and histopathological assays are known in the art for evaluating Alzheimer's disease phenotype, for characterizing therapeutic agents, and assessing treatment. Histological analyses are typically performed postmortem. Histological analysis of Aβ levels may be performed using Thioflavin-S. Congo red, or anti-AB staining (e.g., 4G8, 10D5, or 6E10 antibodies) to visualize AB deposition on sectioned brain tissues (see, e.g., Holcomb et al., 1998, Nat. Med. 4:97-100; Borchelt et al., 1997, Neuron 19:939-945; Dickson et al., 1988, Am. J. Path. 132:86-101). In vivo methods of visualizing Aβ deposition in transgenic mice have been also described. BSB ((trans, trans)-1-bromo-2,5-bis-(3-hydroxycarbonyl-4-hydroxy) styrylbenzene) and PET tracer ¹¹C-labelled Pittsburgh Compound-B (PIB) bind to Aβ plaques (see, e.g., Skovronsky et al., 2000, Proc. Natl. Acad. Sci. USA 97:7609-7614; Klunk et al., 2004, Ann. Neurol. 55:306-319). F-containing amyloidophilic Congo red-type compound FSB ((E,E)-1-fluoro-2,5-bis-(3-hydroxycarbonyl-4-hydroxy) styrylbenzene) allows visualization of Aβ plaques by MRI (see, e.g., Higuchi et al., 2005, Nature Neurosci. 8:527-533). Radiolabeled, putrescine-modified amyloid-beta peptide labels amyloid deposits in vivo in a mouse model of Alzheimer's disease (see, e.g., Wengenack et al., 2000, Nat. Biotechnol. 18:868-872).

Increased glial fibrillary acidic protein (GFAP) by astrocytes is a marker for astroglial activation and gliosis during neurodegeneration. AB plaques are associated with GFAP-positive activated astrocytes, and may be visualized via GFAP staining (see, e.g., Nagele et al. 2004, Neurobiol. Aging 25:663-674; Mandybur et al., 1990, Neurology 40:635-639; Liang et al., 2010, J. Biol. Chem. 285:27737-27744). Neurofibrillary tangles may be identified by immunohistochemistry using thioflavin-S fluorescent microscopy and Gallyas silver stains (see, e.g., Gotz et al., 2001, J. Biol. Chem. 276:529-534; U.S. Pat. No. 6,664,443). Axon staining with electron microscopy and axonal transport studies may be used to neuronal degeneration (see, e.g., Ishihara et al., 1999, Neuron 24:751-762).

Subjects suffering from Alzheimer's disease can be identified using standard diagnostic methods known in the art for Alzheimer's disease. Generally, diagnosis of Alzheimer's disease is based on symptoms (e.g., progressive decline in memory function, gradual retreat from and frustration with normal activities, apathy, agitation or irritability, aggression, anxiety, sleep disturbance, dysphoria, aberrant motor behavior, disinhibition, social withdrawal, decreased appetite, hallucinations, dementia), medical history, neuropsychological tests, neurological and/or physical examination of a patient. Cerebrospinal fluid may also be for tested for various proteins that have been associated with Alzheimer pathology, including tau, amyloid beta peptide, and AD7C-NTP. Genetic testing is also available for early-onset familial Alzheimer disease (eFAD), an autosomal-dominant genetic disease. Clinical genetic testing is available for individuals with AD symptoms or at-risk family members of patients with early-onset disease. In the U.S., mutations for PS2, and APP may be tested in a clinical or federally approved laboratory under the Clinical Laboratory Improvement Amendments. A commercial test for PSI mutations is also available (Elan Pharmaceuticals).

The effectiveness of one or more Compounds selected from the table 1 ab and Table 1c described herein and monitoring of a subject who receives one or more Compounds selected from the table 1 ab and Table 1c can readily be determined by a person skilled in the medical and clinical arts. One or any combination of diagnostic methods, including physical examination, assessment and monitoring of clinical symptoms, and performance of analytical tests and methods described herein, may be used for monitoring the health status of the subject. The effects of administering one or more Compounds selected from the table 1 ab and Table 1c can be analyzed using techniques known in the art, such as comparing symptoms of patients suffering from or at risk of Alzheimer's disease that have received the treatment with those of patients without such a treatment or with placebo treatment.

### Mild Cognitive Impairment (MCI).

MCI is a brain-function syndrome involving the onset and evolution of cognitive impairments beyond those expected based on age and education of the individual, but which are not significant enough to interfere with this individual's daily activities. MCI is an aspect of cognitive aging that is considered to be a transitional state between normal aging and the dementia into which it may convert (see, Pepeu, Dialogues in Clinical Neuroscience 6:369-377, 2004). MCI that primarily affects memory is known as “amnestic MCI.” A person with amnestie MCI may start to forget important information that he or she would previously have recalled easily, such as recent events. Amnestic MCI is frequently seen as prodromal stage of Alzheimer's disease. MCI that affects thinking skills other than memory is known as “non-amnestic MCL.” This type of MCI affect thinking skills such as the ability to make sound decisions, judge the time or sequence of steps needed to complete a complex task, or visual perception. Individuals with non-amnestic MCI are believed to be more likely to convert to other types of dementias (e.g., dementia with Lewy bodies).

Persons in the medical art have a growing recognition that people diagnosed with Parkinson's disease may have MCI in addition to their physical symptoms. Recent studies show 20-30% of people with Parkinson's disease have MCI, and that their MCI tends to be non-amnestic. Parkinson's disease patients with MCI sometimes go on to develop full blown dementia (Parkinson's disease with dementia).

Methods for detecting, monitoring, quantifying or assessing neuropathological deficiencies associated with MCI are known in the art, including astrocyte morphological analyses, release of acetylcholine, silver staining for assessing neurodegeneration, and PiB PET imaging to detect beta amyloid deposits (see, e.g., U.S. Application Publication No. 2012/0071468; Pepeu, 2004, supra). Methods for detecting, monitoring, quantifying or assessing behavioral deficiencies associated with MCI are also known in the art, including eight-arm radial maze paradigm, non-matching-to-sample task, allocentric place determination task in a water maze, Morris maze test, visuospatial tasks, and delayed response spatial memory task, olfactory novelty test (see, id.).

### Motor Neuron Dysfunction (MND).

MND is a group of progressive neurological disorders that destroy motor neurons, the cells that control essential voluntary muscle activity such as speaking, walking, breathing and swallowing. It is classified according to whether degeneration affects upper motor neurons, lower motor neurons, or both. Examples of MNDs include, but are not limited to Amyotrophic Lateral Sclerosis (ALS), also known as Lou Gehrig's Disease, progressive bulbar palsy, pseudobulbar palsy, primary lateral sclerosis, progressive muscular atrophy, lower motor neuron disease, and spinal muscular atrophy (SMA) (e.g., SMA1 also called Werdnig-Hoffman Disease, SMA2, SMA3 also called Kugelberg-Welander Disease, and Kennedy's disease), post-polio syndrome, and hereditary spastic paraplegia. In adults, the most common MND is amyotrophic lateral sclerosis (ALS), which affects both upper and lower motor neurons. It can affect the arms, legs, or facial muscles. Primary lateral sclerosis is a disease of the upper motor neurons, while progressive muscular atrophy affects only lower motor neurons in the spinal cord. In progressive bulbar palsy, the lowest motor neurons of the brain stem are most affected, causing slurred speech and difficulty chewing and swallowing. There are almost always mildly abnormal signs in the arms and legs. Patients with MND exhibit a phenotype of Parkinson's disease (e.g., having tremor, rigidity, bradykinesia, and/or postural instability). Methods for detecting, monitoring or quantifying locomotor and/or other deficits associated with Parkinson's diseases, such as MND, are known in the art (see, e.g., U.S. Application Publication No. 20120005765).

Methods for detecting, monitoring, quantifying or assessing motor deficits and histopathological deficiencies associated with MND are known in the art, including histopathological, biochemical, and electrophysiological studies and motor activity analysis (see, e.g., Rich et al., J Neurophysiol 88:3293-3304, 2002; Appel et al., Proc. Natl. Acad. Sci. USA 88:647-51, 1991). Histopathologically, MNDs are characterized by death of motor neurons, progressive accumulation of detergent-resistant aggregates containing SOD1 and ubiquitin and aberrant neurofilament accumulations in degenerating motor neurons. In addition, reactive astroglia and microglia are often detected in diseased tissue. Patients with an MND show one or more motor deficits, including muscle weakness and wasting, uncontrollable twitching, spasticity, slow and effortful movements, and overactive tendon reflexes.

### Ophthalmic Diseases and Disorders:

In certain embodiments, a senescence-associated disease or disorder is an ocular disease, disorder, or condition, for example, presbyopia, macular degeneration, or cataracts. In other certain embodiments, the senescence-associated disease or disorder is glaucoma. Macular degeneration is a neurodegenerative disease that causes the loss of photoreceptor cells in the central part of retina, called the macula. Macular degeneration generally is classified into two types: dry type and wet type. The dry form is more common than the wet, with about 90% of age-related macular degeneration (ARMD of AMD) patients diagnosed with the dry form. The wet form of the disease usually leads to more serious vision loss. While the exact causes of age-related macular degeneration are still unknown, the number of senescent retinal pigmented epithelial (RPE) cells increases with age. Age and certain genetic factors and environmental factors are risk factors for developing ARMD. Environment predisposing factors include omega-3 fatty acids intake; estrogen exposure; and increased serum levels of vitamin D. Genetic predisposing risk factors include reduced levels Dicer1 (enzyme involved in maturation of micro RNA) in eyes of patients with dry AMD, and decreased micro RNAs contributes to a senescent cell profile; and DICER1 ablation induces premature senescence.

Dry ARMD is associated with atrophy of RPE layer, which causes loss of photoreceptor cells. The dry form of ARMD may result from aging and thinning of macular tissues and from deposition of pigment in the macula. Senescence appears to inhibit both replication and migration of RPE, resulting in permanent RPE depletion in the macula of dry AMD patients. With wet ARMD, new blood vessels grow beneath the retina and leak blood and fluid. This abnormal leaky choroidal neovascularization causes the retinal cells to die, creating blind spots in central vision. Different forms of macular degeneration may also occur in younger patients. Non-age related etiology may be linked to heredity, diabetes, nutritional deficits, bead injury, infection, or other factors.

Declining vision noticed by the patient or by an ophthalmologist during a routine eye exam may be the first indicator of macular degeneration. The formation of exudates, or “drusen,” underneath the Bruch's membrane of the macula is often the first physical sign that macular degeneration may develop. Symptoms include perceived distortion of straight lines and, in some cases, the center of vision appears more distorted than the rest of a scene; a dark, blurry area or “white-out” appears in the center of vision; and/or color perception changes or diminishes. Diagnosing and monitoring of a subject with macular degeneration may be accomplished by a person skilled in the ophthalmic art according to art-accepted periodic eye examination procedures and report of symptoms by the subject.

Presbyopia is an age-related condition where the eye exhibits a progressively diminished ability to focus on near objects as the speed and amplitude of accommodation of a normal eye decreases with advancing age. Loss of elasticity of the crystalline lens and loss of contractility of the ciliary muscles have been postulated as its cause. Age-related changes in the mechanical properties of the anterior lens capsule and posterior lens capsule suggest that the mechanical strength of the posterior lens capsule decreases significantly with age.

The laminated structure of the capsule also changes and may result, at least in part, from a change in the composition of the tissue. The major structural component of the lens capsule is basement membrane type IV collagen that is organized into a three-dimensional molecular network. Type IV collagen is composed of six homologous α chains (α1-6) that associate into heterotrimeric collagen IV protomers with each comprising a specific chain combination of α112, α345, or α556. Protomers share structural similarities of a triple-helical collagenous domain with the triplet peptide sequence of Gly-X-Y, ending in a globular C-terminal region termed the non-collagenous 1 (NC1) domain. The N-termini are composed of a helical domain termed the 7S domain, which is also involved in protomer-protomer interactions.

Research has suggested that collagen IV influences cellular function which is inferred from the positioning of basement membranes underneath epithelial layers, and data support the role of collagen IV in tissue stabilization. Posterior capsule opacification (PCO) develops as a complication in approximately 20-40% of patients in subsequent years after cataract surgery. PCO results from proliferation and activity of residual lens epithelial cells along the posterior capsule in a response akin to wound healing. Growth factors, such as fibroblast growth factor, transforming growth factor β, epidermal growth factor, hepatocyte growth factor, insulin-like growth factor, and interleukins IL-1 and IL-6 may also promote epithelial cell migration. As discussed herein, production of these factors and cytokines by senescent cells contribute to the SASP. In contrast, in vitro studies show that collagen IV promotes adherence of lens epithelial cells. Adhesion of the collagen IV, fibronectin, and laminin to the intraocular lens inhibits cell migration and may reduce the risk of PCO.

Without wishing to be bound by any particular theory, selective killing of senescent cells by the and/or other properties of Compounds selected from the table 1 ab and Table 1c may slow or impede (delay, inhibit, retard) the disorganization of the type IV collagen network. Removal of senescence cells and thereby removing the inflammatory effects of SASP may decrease or inhibit epithelial cell migration and may also delay (suppress) the onset of presbyopia or decrease or slow the progressive severity of the condition (such as slow the advancement from mild to moderate or moderate to severe). The Compounds selected from the table 1 ab and Table 1c described herein may also be useful for post-cataract surgery to reduce the likelihood of occurrence of PCO.

While no direct evidence for the involvement of cellular senescence with the development of cataracts has been obtained from human studies, BubR1 hypomorphic mice develop posterior subcapsular cataracts bilaterally early in life, suggesting that senescence may play a role. Cataracts are a clouding of the lens of an eye, causing blurred vision, and if left untreated can result in blindness. Surgery is effective and routinely performed to remove cataracts. Administration of one or more of the Compounds selected from the table 1 ab and Table 1c described herein may result in decreasing the likelihood of occurrence of a cataract or may slow or inhibit progression of a cataract. The presence and severity of a cataract can be monitored by eye exams using methods routinely performed by a person skilled in the ophthalmology art.

In certain embodiments, at least one compound selected from the table 1 ab and Table 1c that selectively kills senescent cells may be administered to a subject who is at risk of developing presbyopia, cataracts, or macular degeneration. Treatment with a compound selected from the table 1 ab and Table 1c may be initiated when a human subject is at least 40 years of age to delay or inhibit onset or development of cataracts, presbyopia, and macular degeneration. Because almost all humans develop presbyopia, in certain embodiments, the compound selected from the table 1 ab and Table 1c may be administered in a manner as described herein to a human subject after the subject reaches the age of 40 to delay or inhibit onset or development of presbyopia.

In certain embodiments, the senescence associated disease or disorder is glaucoma. Glaucoma is a broad term used to describe a group of diseases that causes visual field loss, often without any other prevailing symptoms. The lack of symptoms often leads to a delayed diagnosis of glaucoma until the terminal stages of the disease. Even if subjects afflicted with glaucoma do not become blind, their vision is often severely impaired. Normally, clear fluid flows into and out of the front part of the eye, known as the anterior chamber. In individuals who have open/wide-angle glaucoma, this fluid drains too slowly, leading to increased pressure within the eye. If left untreated, this high pressure subsequently damages the optic nerve and can lead to complete blindness. The loss of peripheral vision is caused by the death of ganglion cells in the retina. Ganglion cells are a specific type of projection neuron that connects the eye to the brain. When the cellular network required for the outflow of fluid was subjected to SA-β-Gal staining, a fourfold increase in senescence has been observed in glaucoma patients.

For monitoring the effect of a therapy on inhibiting progression of glaucoma, standard automated perimetry (visual field test) is the most widely used technique. In addition, several algorithms for progression detection have been developed (see, e.g., Wesselink et al., Arch Ophthalmol. 127(3):270-274 (2009), and references therein). Additional methods include gonioscopy (examines the trabecular meshwork and the angle where fluid drains out of the eye); imaging technology, for example scanning laser tomography (e.g., HRT3), laser polarimetry (e.g., GDX), and ocular coherence tomography); ophthalmoscopy; and pachymeter measurements that determine central corneal thickness.

### Metabolic Disease or Disorder.

Senescence-associated diseases or disorders treatable by administering a compound selected from the table 1 ab and Table 1c include metabolic diseases or disorders. Such senescent cell associated diseases and disorders include diabetes, metabolic syndrome, diabetic ulcers, and obesity.

Diabetes is characterized by high levels of blood glucose caused by defects in insulin production, insulin action, or both. The great majority (90 to 95%) of all diagnosed cases of diabetes in adults are type 2 diabetes, characterized by the gradual loss of insulin production by the pancreas. Diabetes is the leading cause of kidney failure, nontraumatic lower-limb amputations, and new cases of blindness among adults in the U.S. Diabetes is a major cause of heart disease and stroke and is the seventh leading cause of death in the U.S. (see, e.g., Centers for Disease Control and Prevention, National diabetes fact sheet: national estimates and general information on diabetes and pre-diabetes in the United States, 2011 (“Diabetes fact sheet”). Compounds selected from the table 1 ab and Table 1c described herein may be used for treating type 2 diabetes, particularly age-, diet- and obesity-associated type 2 diabetes.

Involvement of senescent cells in metabolic disease, such as obesity and type 2 diabetes, has been suggested as a response to injury or metabolic dysfunction. Fat tissue from obese mice showed induction of the senescence markers SA-β-Gal, p53, and p21. A concomitant up-regulation of pro-inflammatory cytokines, such as tumor necrosis factor-α and Ccl2/MCP1, was observed in the same fat tissue. Induction of senescent cells in obesity potentially has clinical implications because pro-inflammatory SASP components are also suggested to contribute to type 2 diabetes. A similar pattern of up-regulation of senescence markers and SASP components are associated with diabetes, both in mice and in humans. Accordingly, the methods described herein that comprise administering a compound selected from the table 1 ab and Table 1c may be useful for treatment or prophylaxis of type 2 diabetes, as well as obesity and metabolic syndrome. Without wishing to be bound by theory, contact of senescent pre-adipocytes with a compound selected from the table 1 ab and Table 1c thereby killing the senescent pre-adipocytes may provide clinical and health benefit to a person who has any one of diabetes, obesity, or metabolic syndrome.

Subjects suffering from type 2 diabetes can be identified using standard diagnostic methods known in the art for type 2 diabetes. Generally, diagnosis of type 2 diabetes is based on symptoms (e.g., increased thirst and frequent urination, increased hunger, weight loss, fatigue, blurred vision, slow-healing sores or frequent infections, and/or areas of darkened skin), medical history, and/or physical examination of a patient. Subjects at risk of developing type 2 diabetes include those who have a family history of type 2 diabetes and those who have other risk factors such as excess weight, fat distribution, inactivity, race, age, prediabetes, and/or gestational diabetes.

The effectiveness of a compound selected from the table 1 ab and Table 1c can readily be determined by a person skilled in the medical and clinical arts. One or any combination of diagnostic methods, including physical examination, assessment and monitoring of clinical symptoms, and performance of analytical tests and methods, such as those described herein, may be used for monitoring the health status of the subject. A subject who is receiving one or more Compounds selected from the table 1 ab and Table 1c described herein for treatment or prophylaxis of diabetes can be monitored, for example, by assaying glucose and insulin tolerance, energy expenditure, body composition, fat tissue, skeletal muscle, and liver inflammation, and/or lipotoxicity (muscle and liver lipid by imaging in vivo and muscle, liver, bone marrow, and pancreatic β-cell lipid accumulation and inflammation by histology). Other characteristic features or phenotypes of type 2 diabetes are known and can be assayed as described herein and by using other methods and techniques known and routinely practiced in the art.

Obesity and obesity-related disorders are used to refer to conditions of subjects who have a body mass that is measurably greater than ideal for their height and frame. Body Mass index (BMI) is a measurement tool used to determine excess body weight, and is calculated from the height and weight of a subject. A human is considered overweight when the person has a BMI of 25-29; a person is considered obese when the person has a BMI of 30-39, and a person is considered severely obese when the person has a BMI of ≥40. Accordingly, the terms obesity and obesity-related refer to human subjects with body mass index values of greater than 30, greater than 35, or greater than 40. A category of obesity not captured by BMI is called “abdominal obesity” in the art, which relates to the extra fat found around a subject's middle, which is an important factor in health, even independent of BMI. The simplest and most often used measure of abdominal obesity is waist size. Generally abdominal obesity in women is defined as a waist size 35 inches or higher, and in men as a waist size of 40 inches or higher. More complex methods for determining obesity require specialized equipment, such as magnetic resonance imaging or dual energy X-ray absorptioimetry machines.

A condition or disorder associated with diabetes and senescence is a diabetic ulcer (i.e., diabetic wound). An ulcer is a breakdown in the skin, which may extend to involve the subcutaneous tissue or even muscle or bone. These lesions occur, particularly, on the lower extremities. Patients with diabetic venous ulcer exhibit elevated presence of cellular senescence at sites of chronic wounds. Chronic inflammation is also observed at sites of chronic wounds, such as diabetic ulcers, suggesting that the proinflammatory cytokine phenotype of senescent cells has a role in the pathology.

Subjects who have type 2 diabetes or who are at risk of developing type 2 diabetes may have metabolic syndrome. Metabolic syndrome in humans is typically associated with obesity and characterized by one or more of cardiovascular disease, liver steatosis, hyperlipidemia, diabetes, and insulin resistance. A subject with metabolic syndrome may present with a cluster of metabolic disorders or abnormalities which may include, for example, one or more of hypertension, type-2 diabetes, hyperlipidemia, dyslipidemia (e.g., hypertriglyceridemia, hypercholesterolemia), insulin resistance, liver steatosis (steatohepatitis), hypertension, atherosclerosis, and other metabolic disorders.

### Renal Dysfunction:

Nephrological pathologies, such as glomerular disease, arise in the elderly. Glomerulonephritis is characterized by inflammation of the kidney and by the expression of two proteins, IL1α and IL1β. Glomerular disease is associated with elevated presence of senescent cells, especially in fibrotic kidneys.

### Dermatological Disease or Disorder.

Senescence-associated diseases or disorders treatable by administering a compound selected from the table 1 ab and Table 1c described herein include dermatological diseases or disorders. Such senescent cell associated diseases and disorders include psoriasis and eczema, which are also inflammatory diseases and are discussed in greater detail above. Other dermatological diseases and disorders that are associated with senescence include rhytides (wrinkles due to aging); pruritis (linked to diabetes and aging); dysesthesia (chemotherapy side effect that is linked to diabetes and multiple sclerosis); psoriasis (as noted) and other papulosquamous disorders, for example, erythroderma, lichen planus, and lichenoid dermatosis; atopic dermatitis (a form of eczema and associated with inflammation); eczematous eruptions (often observed in aging patients and linked to side effects of certain drugs). Other dermatological diseases and disorders associated with senescence include eosinophilic dermatosis (linked to certain kinds of hemotologic cancers); reactive neutrophilic dermatosis (associated with underlying diseases such as inflammatory bowel syndrome); pemphigus (an autoimmune disease in which autoantibodies form against desmoglein); pemphigoid and other immunobullous dermatosis (autoimmune blistering of skin); fibrohistocytic proliferations of skin, which is linked to aging; and cutaneous lymphomas that are more common in older populations. Another dermatological disease that may be treatable according to the methods described herein includes cutaneous lupus, which is a symptom of lupus erythematosus. Late onset lupus may be linked to decreased (i.e., reduced) function of T-cell and B-cells and cytokines (immunosenescence) associated with aging.

### Metastasis.

In a particular embodiment, methods are provided for treating or preventing (i.e., reducing the likelihood of occurrence of development of) a senescence cell associated disease (or disorder or condition), which is metastasis. The Compounds selected from the table 1 ab and Table 1c described herein may also be used according to the methods described herein for treating or preventing (i.e., reducing the likelihood of occurrence of) metastasis (i.e., the spreading and dissemination of cancer or tumor cells) from one organ or tissue to another organ or tissue in the body.

A senescent cell-associated disease or disorder includes metastasis, and a subject who has a cancer may benefit from administration of a compound selected from the table 1 ab and Table 1c as described herein for inhibiting metastasis. Such a compound selected from the table 1 ab and Table 1c when administered to a subject who has a cancer according to the methods described herein may inhibit tumor proliferation. Metastasis of a cancer occurs when the cancer cells (i.e., tumor cells) spread beyond the anatomical site of origin and initial colonization to other areas throughout the body of the subject. Tumor proliferation may be determined by tumor size, which can be measured in various ways familiar to a person skilled in the art, such as by PET scanning, MRI, CAT scan, biopsy, for example. The effect of the therapeutic agent on tumor proliferation may also be evaluated by examining differentiation of the tumor cells.

As used herein and in the art, the terms cancer or tumor are clinically descriptive terms that encompass diseases typically characterized by cells exhibiting abnormal cellular proliferation. The term cancer is generally used to describe a malignant tumor or the disease state arising from the tumor. Alternatively, an abnormal growth may be referred to in the art as a neoplasm. The term tumor, such as in reference to a tissue, generally refers to any abnormal tissue growth that is characterized, at least in part, by excessive and abnormal cellular proliferation. A tumor may be metastatic and capable of spreading beyond its anatomical site of origin and initial colonization to other areas throughout the body of the subject. A cancer may comprise a solid tumor or may comprise a “liquid” tumor (e.g., leukemia and other blood cancers).

Cells are induced to senesce by cancer therapies, such as radiation and certain chemotherapy drugs. The presence of senescent cells increases secretion of inflammatory molecules (see description herein of senescent cells), promotes tumor progression, which may include promoting tumor growth and increasing tumor size, promoting metastasis, and altering differentiation. When senescent cells are destroyed, tumor progression is significantly inhibited, resulting in tumors of small size and with little or no observed metastatic growth (see, e.g., Int'l Appl. Publication No. WO 2013/090645).

In one embodiment, methods are provided for preventing (i.e., reducing the likelihood of occurrence of), inhibiting, or retarding metastasis in a subject who has a cancer by administering a compound selected from the table 1 ab and Table 1c as described herein. In a particular embodiment, the compound selected from the table 1 ab and Table 1c is administered on one or more days within a treatment window (i.e., treatment course) of no longer than 7 days or 14 days. In other embodiments, the treatment course is no longer than 2, 3, 4, 5, 6, 7, 8, 9, 10, 11, 12, 13, 14, 15, 16, 17, 18, 19, 20, or no longer than 21 days. In other embodiments, the treatment course is a single day. In certain embodiments, the compound selected from the table 1 ab and Table 1c is administered on two or more days within a treatment window of no longer than 7 days or 14 days, on 3 or more days within a treatment window of no longer than 7 days or 14 days; on 4 or more days within a treatment window of no longer than 7 days or 14 days; on 5 or more days within a treatment window of no longer than 7 days or 14 days; on 6, 7, 8, 9, 10, 11, 12, 13, or 14 days within treatment window of no longer than 7 days or 14 days. In certain embodiments, when the at least one compound selected from the table 1 ab and Table 1c is administered to a subject for a treatment window of 3 days or more, the agent may be administered every 2ªd day (i.e., every other day), In other certain embodiments when the at least one compound selected from the table 1 ab and Table 1c is administered to a subject for a treatment window of 4 days or more, the agent may be administered every 350 day (i.e., every other third day).

Because cells may be induced to senesce by cancer therapies, such as radiation and certain chemotherapy drugs (e.g., doxorubicin; paclitaxel; gemcitabine; pomalidomide; lenalidomide), a compound selected from the table 1 ab and Table 1c described herein may be administered after the chemotherapy or radiotherapy to kill (or facilitate killing) of these senescent cells. As discussed herein and understood in the art, establishment of senescence, such as shown by the presence of a senescence-associated secretory phenotype (SASP), occurs over several days; therefore, administering a compound selected from the table 1 ab and Table 1c to kill senescent cells, and thereby reduce the likelihood of occurrence or reduce the extent of metastasis, is initiated when senescence has been established. As discussed herein, the following treatment courses for administration of the compound selected from the table 1 ab and Table 1c may be used in methods described herein for treating or preventing (i.e., reducing the likelihood of occurrence, or reducing the severity) a chemotherapy or radiotherapy side effect.

In certain embodiments, when chemotherapy or radiotherapy is administered in a treatment cycle of at least one day on-therapy (i.e., chemotherapy or radiotherapy)) followed by at least 3, 4, 5, 6, 7, 8, 9, 10, 11, 12, 13, 14 (or about 2 weeks), 15, 16, 17, 18, 19, 20, 21 (or about 3 weeks) days, or about 4 weeks (about one month) off-therapy (i.e., off chemo- or radio-therapy), the compound selected from the table 1 ab and Table 1c is administered on one or more days during the off-therapy time interval (time period) beginning on or after the second day of the off-therapy time interval and ending on or before the last day of the off-therapy time interval. By way of illustrative example, if n is the number of days off-therapy, then the compound selected from the table 1 ab and Table 1c is administered on at least one day and no more than n−1 days of the off-therapy time interval. In a certain particular embodiment when chemotherapy or radiotherapy is administered in a treatment cycle of at least one day on-therapy (i.e., chemotherapy or radiotherapy)) followed by at least one week off-therapy, the compound selected from the table 1 ab and Table 1c is administered on one or more days during the off-therapy time interval beginning on or after the second day of the off-therapy time interval and ending on or before the last day of the off-therapy time interval. In a more specific embodiment, when chemotherapy or radiotherapy is administered in a treatment cycle of at least one day on-therapy (i.e., chemotherapy or radiotherapy) followed by at least one week off therapy, the compound selected from the table 1 ab and Table 1c is administered on one day that is the sixth day of the off-therapy time interval. In other specific embodiments, when chemotherapy or radiotherapy is administered in a treatment cycle of at least one day on-therapy (i.e., chemotherapy or radiotherapy)) followed by at least two weeks off-therapy, the compound selected from the table 1 ab and Table 1c is administered beginning on the sixth day of the off-chemo- or radio-therapy time interval and ending at least one day or at least two days prior to the first day of a subsequent chemotherapy of radiation therapy treatment course. By way of example, if the off-chemo- or radio-therapy time interval is two weeks, a compound selected from the table 1 ab and Table 1c may be administered on at least one and on no more than 7 days (i.e., 1, 2, 3, 4, 5, 6, or 7 days) of the off-therapy time interval beginning on the sixth day after the chemotherapy or radiotherapy course ends (i.e., the sixth day of the off chemo-radio-therapy interval). When the off-chemo- or radio-therapy time interval is at least three weeks, a compound selected from the table 1 ab and Table 1c may be administered on at least one day and on no more than 14 days (i.e., 1-14 days: 1, 2, 3, 4, 5, 6, 7, 8, 9, 10, 11, 12, 13, or 14 days) of the off-therapy time interval beginning on the sixth day after the chemotherapy or radiotherapy course ends. In other embodiments, depending on the off-chemo-radio-therapy interval, the compound selected from the table 1 ab and Table 1c treatment course is at least one day and no longer than 2, 3, 4, 5, 6, 7, 8, 9, 10, 11, 12, 13, 14, 15, 16, 17, 18, 19, 20, or no more than 21 days (i.e., 1-21 days), provided that administration of the compound selected from the table 1 ab and Table 1c is not concurrent with the chemotherapy or radiotherapy. In certain embodiments, the compound selected from the table 1 ab and Table 1c treatment course is a single day. In certain embodiments, the compound selected from the table 1 ab and Table 1c is administered on two or more days within a treatment window of no longer than 14 days, on 3 or more days within a treatment window of no longer than 14 days; on 4 or more days within a treatment window of no longer than 14 days; on 5 or more days within a treatment window of no longer than 14 days; on 6, 7, 8, 9, 10, 11, 12, 13, or 14 days within treatment window of no longer than 14 days. In certain embodiments, when the at least one compound selected from the table 1 ab and Table 1c is administered to a subject during a treatment course of 3 days or more, the agent may be administered every 2ªd day (i.e., every other day), In other certain embodiments when the at least one compound selected from the table 1 ab and Table 1c is administered to a subject during a treatment course of 4 days or more, the agent may be administered every 3rd day (i.e., every other third day).

Many chemotherapy and radiotherapy treatment regimens comprise a finite number of cycles of on-drug therapy followed by off-drug therapy or comprise a finite timeframe in which the chemotherapy or radiotherapy is administered. Such cancer treatment regimens may also be called treatment protocols. The protocols are determined by clinical trials, drug labels, and clinical staff in conjunction with the subject to be treated. The number of cycles of a chemotherapy or radiotherapy or the total length of time of a chemotherapy or radiotherapy regimen can vary depending on the patient's response to the cancer therapy. The timeframe for such treatment regimens is readily determined by a person skilled in the oncology art. In another embodiment for treating metastasis, a compound selected from the table 1 ab and Table 1c may be administered after the treatment regimen of chemotherapy or radiotherapy has been completed. In a particular embodiment, the compound selected from the table 1 ab and Table 1c is administered after the chemotherapy or radiotherapy has been completed on one or more days within treatment window (i.e., compound selected from the table 1 ab and Table 1c treatment course) of no longer than 14 days. In other embodiments, the compound selected from the table 1 ab and Table 1c treatment course is no longer than 2, 3, 4, 5, 6, 7, 8, 9, 10, 11, 12, 13, 14, 15, 16, 17, 18, 19, 20, or no more than 21 days. In other embodiments, the treatment course is a single day. In certain embodiments, the compound selected from the table 1 ab and Table 1c is administered on two or more days within a treatment window of no longer than 14 days, on 3 or more days within a treatment window of no longer than 14 days; on 4 or more days within a treatment window of no longer than 14 days; on 5 or more days within a treatment window of no longer than 14 days; on 6, 7, 8, 9, 10, 11, 12, 13, or 14 days within treatment window of no longer than 14 days. In certain embodiments, when the at least one compound selected from the table 1 ab and Table 1c is administered to a subject after chemotherapy or radiotherapy for a treatment window of 3 days or more, the agent may be administered every 2^{nd} day (i.e., every other day). In other certain embodiments when the at least one compound selected from the table 1 ab and Table 1c is administered to a subject for a treatment window of 4 days or more, the agent may be administered every 3^{rd} day (i.e., every other third day). In one embodiment, the treatment with the compound selected from the table 1 ab and Table 1c may be initiated at least 1, 2, 3, 4, 5, 6, 7, 8, 9, 10, 11, 12, 13, or 14 days or later after the cancer treatment regimen has been completed. In a more particular embodiment, the treatment with the compound selected from the table 1 ab and Table 1c may be initiated at least 6, 7, 8, 9, 10, 11, 12, 13, or 14 days or later after the cancer treatment regimen has been completed. Any of the additional treatment courses and treatment cycles for administration of a compound selected from the table 1 ab and Table 1c described herein may be followed for inhibiting metastasis in a subject after a chemotherapy or radiotherapy protocol has been completed.

A chemotherapy may be referred to as a chemotherapy, chemotherapeutic, or chemotherapeutic drug. Many chemotherapeutics are compounds referred to as small organic molecules. Chemotherapy is a term that is also used to describe a combination chemotherapeutic drugs that are administered to treat a particular cancer. As understood by a person skilled in the art, a chemotherapy may also refer to a combination of two or more chemotherapeutic molecules that are administered coordinately and which may be referred to as combination chemotherapy. Numerous chemotherapeutic drugs are used in the oncology art and include, without limitation, alkylating agents; antimetabolites; anthracyclines, plant alkaloids; and topoisomerase inhibitors,

A cancer that may metastasize may be a solid tumor or may be a liquid tumor (e.g., a blood cancer, for example, a leukemia). Cancers that are liquid tumors are classified in the art as those that occur in blood, bone marrow, and lymph nodes and include generally, leukemias (myeloid and lymphocytic), lymphomas (e.g., Hodgkin lymphoma), and melanoma (including multiple myeloma). Leukemias include for example, acute lymphoblastic leukemia (ALL), acute myeloid leukemia (AML), chronic lymphocytic leukemia (CLL), chronic myelogenous leukemia (CML), and hairy cell leukemia. Cancers that are solid tumors and occur in greater frequency in humans include, for example, prostate cancer, testicular cancer, breast cancer, brain cancer, pancreatic cancer, colon cancer, thyroid cancer, stomach cancer, lung cancer, ovarian cancer, Kaposi's sarcoma, skin cancer (including squamous cell skin cancer), renal cancer, head and neck cancers, throat cancer, squamous carcinomas that form on the moist mucosal linings of the nose, mouth, throat, etc.), bladder cancer, osteosarcoma (bone cancer), cervical cancer, endometrial cancer, esophageal cancer, liver cancer, and kidney cancer. In certain specific embodiments, the senescent cell-associated disease or disorder treated or prevented (i.e., likelihood of occurrence or development is reduced) by the methods described herein is metastasis of melanoma cells, prostate cancer cells, testicular cancer cells, breast cancer cells, brain cancer cells, pancreatic cancer cells, colon cancer cells, thyroid cancer cells, stomach cancer cells, lung cancer cells, ovarian cancer cells, Kaposi's sarcoma cells, skin cancer cells, renal cancer cells, head or neck cancer cells, throat cancer cells, squamous carcinoma cells, bladder cancer cells, osteosarcoma cells, cervical cancer cells, endometrial cancer cells, esophageal cancer cells, liver cancer cells, or kidney cancer cells.

The methods described herein are also useful for inhibiting, retarding or slowing progression of metastatic cancer of any one of the types of tumors described in the medical art. Types of cancers (tumors) include the following: adrenocortical carcinoma, childhood adrenocortical carcinoma, aids-related cancers, anal cancer, appendix cancer, basal cell carcinoma, childhood basal cell carcinoma, bladder cancer, childhood bladder cancer, bone cancer, brain tumor, childhood astrocytomas, childhood brain stem glioma, childhood central nervous system atypical teratoid/rhabdoid tumor, childhood central nervous system embryonal tumors, childhood central nervous system germ cell tumors, childhood craniopharyngioma brain tumor, childhood ependymoma brain tumor, breast cancer, childhood bronchial tumors, carcinoid tumor, childhood carcinoid tumor, gastrointestinal carcinoid tumor, carcinoma of unknown primary, childhood carcinoma of unknown primary, childhood cardiac (heart) tumors, cervical cancer, childhood cervical cancer, childhood chordoma, chronic myeloproliferative disorders, colon cancer, colorectal cancer, childhood colorectal cancer, extrahepatic bile duct cancer, ductal carcinoma in situ (DCIS), endometrial cancer, esophageal cancer, childhood esophageal cancer, childhood esthesioneuroblastoma, eye cancer, malignant fibrous histiocytoma of bone, gallbladder cancer, gastric (stomach) cancer, childhood gastric (stomach) cancer, gastrointestinal stromal tumors (GIST), childhood gastrointestinal stromal tumors (GIST), childhood extracranial germ cell tumor, extragonadal germ cell tumor, gestational trophoblastic tumor, glioma, head and neck cancer, childhood head and neck cancer, hepatocellular (liver) cancer, hypopharyngeal cancer, kidney cancer, renal cell kidney cancer, Wilms tumor, childhood kidney tumors, Langerhans cell histiocytosis, laryngeal cancer, childhood laryngeal cancer, leukemia, acute lymphoblastic leukemia (ALL), acute myeloid leukemia (AML), chronic lymphocytic leukemia (CLL), chronic myelogenous leukemia (cml), hairy cell leukemia, lip cancer, liver cancer (primary), childhood liver cancer (primary), lobular carcinoma in situ (LCIS), lung cancer, non-small cell lung cancer, small cell lung cancer, lymphoma, aids-related lymphoma, burkitt lymphoma, cutaneous t-cell lymphoma, Hodgkin lymphoma, non-Hodgkin lymphoma, primary central nervous system lymphoma (CNS), melanoma, childhood melanoma, intraocular (eye) melanoma, Merkel cell carcinoma, malignant mesothelioma, childhood malignant mesothelioma, metastatic squamous neck cancer with occult primary, midline tract carcinoma involving NUTgene, mouth cancer, childhood multiple endocrine neoplasia syndromes, mycosis fungoides, myelodysplastic syndromes, myelodysplastic neoplasms, myeloproliferative neoplasms, multiple myeloma, nasal cavity cancer, nasopharyngeal cancer, childhood nasopharyngeal cancer, neuroblastoma, oral cancer, childhood oral cancer, oropharyngeal cancer, ovarian cancer, childhood ovarian cancer, epithelial ovarian cancer, low malignant potential tumor ovarian cancer, pancreatic cancer, childhood pancreatic cancer, pancreatic neuroendocrine tumors (islet cell tumors), childhood papillomatosis, paraganglioma, paranasal sinus cancer, parathyroid cancer, penile cancer, pharyngeal cancer, pheochromocytoma, pituitary tumor, plasma cell neoplasm, childhood pleuropulmonary blastoma, prostate cancer, rectal cancer, renal pelvis transitional cell cancer, retinoblastoma, salivary gland cancer, childhood salivary gland cancer, Ewing sarcoma family of tumors, Kaposi Sarcoma, osteosarcoma, rhabdomyosarcoma, childhood rhabdomyosarcoma, soft tissue sarcoma, uterine sarcoma, Sezary syndrome, childhood skin cancer, nonmelanoma skin cancer, small intestine cancer, squamous cell carcinoma, childhood squamous cell carcinoma, testicular cancer, childhood testicular cancer, throat cancer, thymoma and thymic carcinoma, childhood thymoma and thymic carcinoma, thyroid cancer, childhood thyroid cancer, ureter transitional cell cancer, urethral cancer, endometrial uterine cancer, vaginal cancer, vulvar cancer, Waldenström macroglobulinemia.

### Chemotherapy and Radiotherapy Side Effects.

In another embodiment, the senescence cell associated disorder or condition is a chemotherapeutic side effect or a radiotherapy side effect. Examples of chemotherapeutic agents that include non-cancer cells to senesce include anthracyclines (such as doxorubicin, daunorubicin); taxols (e.g., paclitaxel); gemcitabine; pomalidomide; and lenalidomide. One or more of the Compounds selected from the table 1 ab and Table 1c administered as described herein may be used for treating and/or preventing (i.e., reducing the likelihood of occurrence of) a chemotherapeutic side effect or a radiotherapy side effect. Removal or destruction of senescent cells may ameliorate acute toxicity, including acute toxicity comprising energy imbalance, of a chemotherapy or radiotherapy. Acute toxic side effects include but are not limited to gastrointestinal toxicity (e.g., nausea, vomiting, constipation, anorexia, diarrhea), peripheral neuropathy, fatigue, malaise, low physical activity, hematological toxicity (e.g., anemia), hepatotoxicity, alopecia (hair loss), pain, infection, mucositis, fluid retention, dermatological toxicity (e.g., rashes, dermatitis, hyperpigmentation, urticaria, photosensitivity, nail changes), mouth (e.g., oral mucositis), gum or throat problems, or any toxic side effect caused by a chemotherapy or radiotherapy. For example, toxic side effects caused by radiotherapy or chemotherapy (see, e.g., National Cancer Institute web site) may be ameliorated by the methods described herein. Accordingly, in certain embodiments, methods are provided herein for ameliorating (reducing, inhibiting, or preventing occurrence (i.e., reducing the likelihood of occurrence)) acute toxicity or reducing severity of a toxic side effect (i.e., deleterious side effect) of a chemotherapy or radiotherapy or both in a subject who receives the therapy, wherein the method comprises administering to the subject an agent that selectively kills, removes, or destroys or facilitates selective destruction of senescent cells. Administration of a compound selected from the table 1 ab and Table 1c for treating or reducing the likelihood of occurrence, or reducing the severity of a chemotherapy or radiotherapy side effect may be accomplished by the same treatment courses described above for treatment/prevention of metastasis. As described for treating or preventing (i.e., reducing the likelihood of occurrence of) metastasis, the compound selected from the table 1 ab and Table 1c is administered during the off-chemotherapy or off-radiotherapy time interval or after the chemotherapy or radiotherapy treatment regimen has been completed.

In a more specific embodiment, the acute toxicity is an acute toxicity comprising energy imbalance and may comprise one or more of weight loss, endocrine change(s) (e.g., hormone imbalance, change in hormone signaling), and change(s) in body composition. In certain embodiments, an acute toxicity comprising energy imbalance relates to decreased or reduced ability of the subject to be physically active, as indicated by decreased or diminished expenditure of energy than would be observed in a subject who did not receive the medical therapy. By way of non-limiting example, such an acute toxic effect that comprises energy imbalance includes low physical activity. In other particular embodiments, energy imbalance comprises fatigue or malaise.

In one embodiment, a chemotherapy side effect to be treated or prevented (i.e., likelihood of occurrence is reduced) by a compound selected from the table 1 ab and Table 1c is cardiotoxicity. A subject who has a cancer that is being treated with an anthracycline (such as doxorubicin, daunorubicin) may be treated with one or more Compounds selected from the table 1 ab and Table 1c described herein that reduce, ameliorate, or decrease the cardiotoxicity of the anthracycline. As is well understood in the medical art, because of the cardiotoxicity associated with anthracyclines, the maximum lifetime dose that a subject can receive is limited even if the cancer is responsive to the drug. Administration of one or more of the Compounds selected from the table 1 ab and Table 1c may reduce the cardiotoxicity such that additional amounts of the anthracycline can be administered to the subject, resulting in an improved prognosis related to cancer disease. In one embodiment, the cardiotoxicity results from administration of an anthracyline, such as doxorubicin. Doxorubicin is an anthracycline topoisomerase that is approved for treating patients who have ovarian cancer after failure of a platinum based therapy; Kaposi's sarcoma after failure of primary systemic chemotherapy or intolerance to the therapy; or multiple myeloma in combination with bortezomib in patients who have not previously received bortezomib or who have received at least one prior therapy. Doxorubicin may cause myocardial damage that could lead to congestive heart failure if the total lifetime dose to a patient exceeds 550 mg/m². Cardiotoxicity may occur at even lower doses if the patient also receives mediastinal irradiation or another cardiotoxic drug. See drug product inserts (e.g., DOXIL, ADRIAMYCIN).

In other embodiments, a compound selected from the table 1 ab and Table 1c may be used in the methods as provided herein for ameliorating chronic or long term side effects. Chronic toxic side effects typically result from multiple exposures to or administrations of a chemotherapy or radiotherapy over a longer period of time. Certain toxic effects appear long after treatment (also called late toxic effects) and result from damage to an organ or system by the therapy. Organ dysfunction (e.g., neurological, pulmonary, cardiovascular, and endocrine dysfunction) has been observed in patients who were treated for cancers during childhood. Without wishing to be bound by any particular theory, by destroying senescent cells, particular normal cells that have been induced to senescence by chemotherapy or radiotherapy, the likelihood of occurrence of a chronic side effect may be reduced, or the severity of a chronic side effect may be reduced or diminished, or the time of onset of a chronic side effect may be delayed. Chronic and/or late toxic side effects that occur in subjects who received chemotherapy or radiation therapy include by way of non-limiting example, cardiomyopathy, congestive heart disease, inflammation, early menopause, osteoporosis, infertility, impaired cognitive function, peripheral neuropathy, secondary cancers, cataracts and other vision problems, hearing loss, chronic fatigue, reduced lung capacity, and lung disease.

In addition, by killing or removing senescent cells in a subject who has a cancer by administering a Compound selected from the table 1 ab and Table 1c, the sensitivity to the chemotherapy or the radiotherapy may be enhanced in a clinically or statistically significant manner than if the compound selected from the table 1 ab and Table 1c was not administered. In some embodiments, development of chemotherapy or radiotherapy resistance may be inhibited when a compound selected from the table 1 ab and Table 1c is administered to a subject treated with the respective chemotherapy or radiotherapy.

### Age-Related Diseases and Disorders.

A compound selected from the table 1 ab and Table 1c may also be useful for treating or preventing (i.e., reducing the likelihood of occurrence) of an age-related disease or disorder that occurs as part of the natural aging process or that occurs when the subject is exposed to a senescence inducing agent or factor (e.g., irradiation, chemotherapy, smoking tobacco, high-fat/high sugar diet, other environmental factors). An age-related disorder or disease or an age-sensitive trait may be associated with a senescence-inducing stimulus. The efficacy of a method of treatment described herein may be manifested by reducing the number of symptoms of an age-related disorder or age-sensitive trait associated with a senescence-inducing stimulus, decreasing the severity of one or more symptoms, or delaying the progression of an age-related disorder or age-sensitive trait associated with a senescence-inducing stimulus. In other particular embodiments, preventing an age-related disorder or age-sensitive trait associated with a senescence-inducing stimulus refers to preventing (i.e., reducing the likelihood of occurrence) or delaying onset of an age-related disorder or age-sensitive trait associated with a senescence-inducing stimulus, or reoccurrence of one or more age-related disorder or age-sensitive trait associated with a senescence-inducing stimulus. Age related diseases or conditions include, for example, renal dysfunction, kyphosis, herniated intervertebral disc, frailty, hair loss, hearing loss, vision loss (blindness or impaired vision), muscle fatigue, skin conditions, skin nevi, diabetes, metabolic syndrome, and sarcopenia. Vision loss refers to the absence of vision when a subject previously had vision. Various scales have been developed to describe the extent of vision and vision loss based on visual acuity. Age-related diseases and conditions also include dermatological conditions, for example without limitation, treating one or more of the following conditions: wrinkles, including superficial fine wrinkles; hyperpigmentation; scars; keloid; dermatitis; psoriasis; eczema (including seborrheic eczema); rosacea; vitiligo; ichthyosis vulgaris; dermatomyositis; and actinic keratosis.

In some embodiments, frailty is defined as a clinically recognizable state of increased vulnerability resulting from aging-associated decline in reserve and function across multiple physiologic systems that compromise a subject's ability to cope with every day or acute stressors. Frailty has been may be characterized by compromised energetics characteristics such as low grip strength, low energy, slowed waking speed, low physical activity, and/or unintentional weight loss. Studies have suggested that a patient may be diagnosed with frailty when three of five of the foregoing characteristics are observed (see, e.g., Fried et al., J. Gerontol. A Biol. Sci. Med. Sci. 2001; 56(3):M146-M156; Xue, Clin. Geriatr. Med. 2011; 27(1):1-15). In certain embodiments, aging and diseases and disorders related to aging may be treated or prevented (i.e., the likelihood of occurrence of is reduced) by administering a Compound selected from the table 1 ab and Table 1c. In some embodiments, the compound selected from the table 1 ab and Table 1c may inhibit senescence of adult stem cells or inhibit accumulation, kill, or facilitate removal of adult stem cells that have become senescent. See, e.g., Park et al., J. Clin. Invest. 113:175-79 (2004) and Sousa-Victor, Nature 506:316-21 (2014) describing importance of preventing senescence in stem cells to maintain regenerative capacity of tissues.

The effectiveness of a compound selected from the table 1 ab and Table 1c with respect to treating a senescence-associated disease or disorder described herein can readily be determined by a person skilled in the medical and clinical arts. One or any combination of diagnostic methods appropriate for the particular disease or disorder, which methods are well known to a person skilled in the art, including physical examination, patient self-assessment, assessment and monitoring of clinical symptoms, performance of analytical tests and methods, including clinical laboratory tests, physical tests, and exploratory surgery, for example, may be used for monitoring the health status of the subject and the effectiveness of the Compound selected from the table 1 ab and Table 1c. The effects of the methods of treatment described herein can be analyzed using techniques known in the art, such as comparing symptoms of patients suffering from or at risk of a particular disease or disorder that have received the pharmaceutical composition comprising a compound selected from the table 1 ab and Table 1c with those of patients who were not treated with the compound selected from the table 1 ab and Table 1c or who received a placebo treatment.

As understood by a person skilled in the medical art, the terms, “treat” and “treatment,” refer to medical management of a disease, disorder, or condition of a subject (i.e., patient) (see, e.g., Stedman's Medical Dictionary). In general, an appropriate dose and treatment regimen provide the compound selected from the table 1 ab and Table 1c in an amount sufficient to provide therapeutic and/or prophylactic benefit. Therapeutic benefit for subjects to whom the Compounds selected from the table 1 ab and Table 1c described herein are administered, includes, for example, an improved clinical outcome, wherein the object is to prevent or slow or retard (lessen) an undesired physiological change associated with the disease, or to prevent or slow or retard (lessen) the expansion of severity of such disease. As discussed herein, effectiveness of the one or more Compounds selected from the table 1 ab and Table 1c may include beneficial or desired clinical results that comprise, but are not limited to, abatement, lessening, or alleviation of symptoms that result from or are associated with the disease to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease progression; amelioration or palliation of the disease state; and remission (whether partial or total), whether detectable or undetectable; and/or overall survival. The effectiveness of the Compounds selected from the table 1 ab and Table 1c described herein may also mean prolonging survival when compared to expected survival if a subject were not receiving the compound selected from the table 1 ab and Table 1c that selectively kills senescent cells.

Administration of a compound selected from the table 1 ab and Table 1c described herein can prolong prolonging survival when compared to expected survival if a subject were not receiving treatment. Subjects in need of treatment include those who already have the disease or disorder as well as subjects prone to have or at risk of developing the disease or disorder, and those in which the disease, condition, or disorder is to be treated prophylactically. A subject may have a genetic predisposition for developing a disease or disorder that would benefit from clearance of senescent cells or may be of a certain age wherein receiving a compound selected from the table 1 ab and Table 1c would provide clinical benefit to delay development or reduce severity of a disease, including an age-related disease or disorder.

In some embodiments, since there are variety of the evidence that senolytes are effective treatment in many disease models due to the their senolytic properties, the fact that compound selected from the table 1 ab and Table 1c is senolytic is enough to prove that it is effective in all and any one of senescence related diseases and conditions as mentioned in this application, as well as in diseases and conditions not mentioned here.

Accordingly, the present invention also relates to the following items

### Items:

1. The compound selected from the group consisting of all compounds listed in the Table 1 ab and Table 1c,
   or its pharmaceutically acceptable salt or solvate, hydrate; tautomer, geometric, optical and stereoisomer thereof, structural analog, functional analog, derivative, prodrug, or compound, having the similar SAR characteristics, mixture thereof in all ratios or combination thereof in all ratios for the use as anti-aging therapy.
2. The compound selected from the group consisting of all compounds listed in the Table 1 ab and Table 1c,
   for ameliorating at list one symptom of the disorder selected from the group, consisting of aging, frailty, senescence, aging related disease, aging related condition, senescence related disease.
3. An anti-aging pharmaceutical composition, comprising a compound selected from the group consisting of all compounds listed in the Table 1 ab and Table 1c.
4. A pharmaceutical composition provided as part of an anti-aging treatment or for treating or preventing an age-related disease or disorder comprising an agent configured to bind to, inhibit, or degrade a protein selected from the group consisting of CDK7, CHEK2, CAPN1, CTSB, HSP90AB1, HSP90AA1, HSPA5, NFKB1, NPC1, PABPC1, ABCB11, ABCB1, PLK1, PLK3, ADRA2A, ADRA2C, ADRA2B, TUBB6, SENP8, SENP6, SENP7, MMP14, MMP13, MMP8, MMP3, MMP1, MAPKAPK2, CTSL, CTSK, CTSS, ATXN2, TBXAS1, CREBBP, PDGFRA, FLT4, FLT1, KDR, PDGFRB, FLT3, KIT, CSF1R, IKBKB, IKBKE, PGGT1B, FAAH, GFER, MAPK14, MAPK12, PRKAA1, ABCG2, ADAM17, BACE2, BACE1, TUBB6, CA7, CA2, CA1, CA5A, CA12, CA9, CA4, CTSB, FEN1, HDAC1, HDAC2, HDAC3, CBX1, SLC6A4, SLC6A2, SLC6A3, PPARD, PPARG, PPARA, PSEN2, HTR7, ADRA1B, SMN2, FYN, SRC, LCK, LYN, TARDBP, CLK2, CLK4, USP2, GBA, RECQL, BLM, WRN, CDK2, PRKAG3, TFPI, LTA4H, DPP7, BACE2, BACE1, CAPN1, CTSB, IGF1R, INSR, EYA2, HSPA5, NFKB1, TCF7L2, TARDBP, BRD4, ABCB1, ADRA1A, ADRA1B, ADRA1D, BRD2, BRD3, BRD4, DRD2, DRD3, DRD4, DRD5, EBP, EGFR, EPHA6, EPHA8, ERBB2, HDAC1, HDAC10, HDAC11, HDAC2, HDAC3, HDAC4, HDAC5, HDAC6, HDAC7, HDAC8, HDAC9, HRH1, HSP90AA1, HTR2A, HTR2B, HTR2C, HTR5B, HTR6, HTR7, KCNH2, MAP4K5, PIK3C2A, PIK3C2B, PIK3CA, PIK3CB, PIK3CD, PIK3CG, PIK3R1, PRKDC, SLC6A2, SLC6A3, SLC6A4 and at least one pharmaceutically acceptable excipient.
5. A pharmaceutical composition provided as part of an anti-aging treatment or for treating or preventing an age-related disease or disorder comprising a compound selected from the group consisting of all compounds listed in the Table 1 ab and Table 1c and at least one pharmaceutically acceptable excipient.
6. An anti-aging pharmaceutical composition, comprising a human cell, modified by the compound selected from the group consisting of all compounds listed in the Table 1 ab and Table 1c. In some embodiments, an anti-aging pharmaceutical composition, comprising a human cell, modified by the compound selected from the group consisting of all compounds listed in the Table 1 ab and Table 1c.
7. A method of providing an anti-aging treatment or of treating or preventing an age-related disease or disorder of a subject comprising reducing, inhibiting, or degrading a gene or protein selected from the group consisting of: CDK7, CHEK2, CAPN1, CTSB, HSP90AB1, HSP90AA1, HSPA5, NFKB1, NPC1, PABPC1, ABCB11, ABCB1, PLK1, PLK3, ADRA2A, ADRA2C, ADRA2B, TUBB6, SENP8, SENP6, SENP7, MMP14, MMP13, MMP8, MMP3, MMP1, MAPKAPK2, CTSL, CTSK, CTSS, ATXN2, TBXAS1, CREBBP, PDGFRA, FLT4, FLT1, KDR, PDGFRB, FLT3, KIT, CSF1R, IKBKB, IKBKE, PGGT1B, FAAH, GFER, MAPK14, MAPK12, PRKAA1, ABCG2, ADAM17, BACE2, BACE1, TUBB6, CA7, CA2, CA1, CA5A, CA12, CA9, CA4, CTSB, FEN1, HDAC1, HDAC2, HDAC3, CBX1, SLC6A4, SLC6A2, SLC6A3, PPARD, PPARG, PPARA, PSEN2, HTR7, ADRA1B, SMN2, FYN, SRC, LCK, LYN, TARDBP, CLK2, CLK4, USP2, GBA, RECQL, BLM, WRN, CDK2, PRKAG3, TFPI, LTA4H, DPP7, BACE2, BACE1, CAPN1, CTSB, IGF1R, INSR EYA2, HSPA5, NFKB1, TCF7L2, TARDBP, BRD4, ABCB1, ADRA1A, ADRA1B, ADRA1D, BRD2, BRD3, BRD4, DRD2, DRD3, DRD4, DRD5, EBP, EGFR, EPHA6, EPHA8, ERBB2, HDAC1, HDAC10, HDAC11, HDAC2, HDAC3, HDAC4, HDAC5, HDAC6, HDAC7, HDAC8, HDAC9, HRH1, HSP90AA1, HTR2A, HTR2B, HTR2C, HTR5B, HTR6, HTR7, KCNH2, MAP4K5, PIK3C2A, PIK3C2B, PIK3CA, PIK3CB, PIK3CD, PIK3CG, PIK3R1, PRKDC, SLC6A2, SLC6A3, SLC6A4 in the organism of the subject, including but not limited to the compound selected from the group consisting of all compounds listed in the Table 1 ab and Table 1c.
8. The method of any one of preceding items, further comprising administering to the subject a gene therapy.
9. The method of any one of preceding items, wherein the age-related disease or disorder is associated with an alleviated level of a protein selected from the group consisting of: CDK7, CHEK2, CAPN1, CTSB, HSP90AB1, HSP90AA1, HSPA5, NFKB1, NPC1, PABPC1, ABCB11, ABCB1, PLK1, PLK3, ADRA2A, ADRA2C, ADRA2B, TUBB6, SENP8, SENP6, SENP7, MMP14, MMP13, MMP8, MMP3, MMP1, MAPKAPK2, CTSL, CTSK, CTSS, ATXN2, TBXAS1, CREBBP, PDGFRA, FLT4, FLT1, KDR, PDGFRB, FLT3, KIT, CSF1R, IKBKB, IKBKE, PGGT1B, FAAH, GFER, MAPK14, MAPK12, PRKAA1, ABCG2, ADAM17, BACE2, BACE1, TUBB6, CA7, CA2, CA1, CA5A, CA12, CA9, CA4, CTSB, FEN1, HDAC1, HDAC2, HDAC3, CBX1, SLC6A4, SLC6A2, SLC6A3, PPARD, PPARG, PPARA, PSEN2, HTR7, ADRA1B, SMN2, FYN, SRC, LCK, LYN, TARDBP, CLK2, CLK4, USP2, GBA, RECQL, BLM, WRN, CDK2, PRKAG3, TFPI, LTA4H, DPP7, BACE2, BACE1, CAPN1, CTSB, IGF1R, INSR, EYA2, HSPA5, NFKB1, TCF7L2, TARDBP, BRD4, ABCB1, ADRA1A, ADRA1B, ADRA1D, BRD2, BRD3, BRD4, DRD2, DRD3, DRD4, DRD5, EBP, EGFR, EPHA6, EPHA8, ERBB2, HDAC1, HDAC10, HDAC11, HDAC2, HDAC3, HDAC4, HDAC5, HDAC6, HDAC7, HDAC8, HDAC9, HRH1, HSP90AA1, HTR2A, HTR2B, HTR2C, HTR5B, HTR6, HTR7, KCNH2, MAP4K5, PIK3C2A, PIK3C2B, PIK3CA, PIK3CB, PIK3CD, PIK3CG, PIK3R1, PRKDC, SLC6A2, SLC6A3, SLC6A4 in an organism of the subject or with the increased activity of such protein.
10. The method of any one of preceding items, wherein the age-related disease or disorder is selected from the group consisting of frailty, Alzheimer's disease, Parkinson's disease, Huntington's diseases, cardiovascular disease, renal failure, muscle wasting or cachexia, osteopenia or osteoporosis, obesity, insulin resistance or diabetes, diverse adult-onset cancers, atherosclerosis, cardiovascular disease, adult cancer, arthritis, cataracts, osteoporosis, type 2 diabetes, hypertension, age-progressive dementia; amyotrophic lateral sclerosis, stroke, atrophic gastritis, osteoarthritis, NASH, camptocormia, chronic obstructive pulmonary disease, coronary artery disease, dopamine dysregulation syndrome, metabolic syndrome, effort incontinence, Hashimoto's thyroiditis, heart failure, late life depression, immunosenescence, age related decline in immune response to vaccines, age related decline in response to immunotherapy, myocardial infarction, acute coronary syndrome, sarcopenia, sarcopenic obesity, senile osteoporosis, urinary incontinence, stroke, atrophic gastritis, camptocormia, chronic obstructive pulmonary disease, coronary artery disease, dopamine dysregulation syndrome, late life depression, osteoarthritis, chronic fatigue syndrome, senile dementia, mild cognitive impairment due to aging, Creutzfeldt-Jakob disease, stroke, CNS cerebral senility, pre-diabetes, diabetes, peripheral arterial disease, aortic valve disease, stroke, Lewy body disease, progressive subcortical gliosis, progressive supranuclear palsy, thalamic degeneration syndrome, hereditary aphasia, myoclonus epilepsy, macular degeneration, pressure ulcers, delirium, progressive subcortical gliosis, progressive supranuclear palsy, thalamic degeneration syndrome, hereditary aphasia, myoclonus epilepsy, and metabolic disorder.
11. The method of any one of preceding items, wherein the anti-aging treatment is selected from the group consisting of a treatment leading to prevention, amelioration or lessening at least one effect of aging; prevention, amelioration or lessening at least one symptom of aging, prevention, amelioration or lessening at least one symptom of age related disease or condition, decreasing or delaying an increase in a biological age of the subject; slowing a rate of aging of the subject; prevention, amelioration or lessening the effects of frailty; prevention, amelioration or lessening the effects of at least one of an aging-related disease or conditions; increasing a health span or lifespan of the subject; increasing a stress resistance or resilience of the subject; increasing a rate or other enhancement of recovery after surgery, radiotherapy, disease and/or any other stress; prevention, amelioration or lesion the effects of menopausal syndrome; restoring reproductive function; elimination or lessening the spread of senescent cells; modulation of at least one biomarker of aging into the healthier state; a decrease in a rate of wrinkle development; and decrease in a rate of hair greying.
12. The method of any one of preceding items, wherein the anti-aging treatment is a treatment leading to changing to a healthier state a parameter selected from the group consisting of a blood parameter, a heart rate, a cognitive function, a bone density, a basal metabolic rate, a systolic blood pressure, a heel bone mineral density (BMD), a heel quantitative ultrasound index (QUI), a heel broadband ultrasound attenuation, a forced expiratory volume in 1-second (FEV1), forced vital capacity (FVC), a peak expiratory flow (PEF), a duration to first press of snap-button in each round, a reaction time, a mean time to correctly identify matches, a right or left hand grip strength, a whole body fat-free mass, a leg fat-free mass, a time for recovery after a stress-inducing event, a resistance to radiation, a morbidity risk, and a mortality risk of the subject.
13. A method of providing an anti-aging treatment or of treating or preventing an age-related disease or disorder of a subject, comprising administering to the subject a pharmaceutical composition comprising an inhibitor of a protein selected from the group consisting of: CDK7, CHEK2, CAPN1, CTSB, HSP90AB1, HSP90AA1, HSPA5, NFKB1, NPC1, PABPC1, ABCB11, ABCB1, PLK1, PLK3, ADRA2A, ADRA2C, ADRA2B, TUBB6, SENP8, SENP6, SENP7, MMP14, MMP13, MMP8, MMP3, MMP1, MAPKAPK2, CTSL, CTSK, CTSS, ATXN2, TBXAS1, CREBBP, PDGFRA, FLT4, FLT1, KDR, PDGFRB, FLT3, KIT, CSF1R, IKBKB, IKBKE, PGGT1B, FAAH, GFER, MAPK14, MAPK12, PRKAA1, ABCG2, ADAM17, BACE2, BACE1, TUBB6, CA7, CA2, CA1, CA5A, CA12, CA9, CA4, CTSB, FEN1, HDAC1, HDAC2, HDAC3, CBX1, SLC6A4, SLC6A2, SLC6A3, PPARD, PPARG, PPARA, PSEN2, HTR7, ADRA1B, SMN2, FYN, SRC, LCK, LYN, TARDBP, CLK2, CLK4, USP2, GBA, RECQL, BLM, WRN, CDK2, PRKAG3, TFPI, LTA4H, DPP7, BACE2, BACE1, CAPN1, CTSB, IGF1R, INSR, EYA2, HSPA5, NFKB1, TCF7L2, TARDBP, BRD4, ABCB1, ADRA1A, ADRA1B, ADRA1D, BRD2, BRD3, BRD4, DRD2, DRD3, DRD4, DRD5, EBP, EGFR, EPHA6, EPHA8, ERBB2, HDAC1, HDAC10, HDAC11, HDAC2, HDAC3, HDAC4, HDAC5, HDAC6, HDAC7, HDAC8, HDAC9, HRH1, HSP90AA1, HTR2A, HTR2B, HTR2C, HTR5B, HTR6, HTR7, KCNH2, MAP4K5, PIK3C2A, PIK3C2B, PIK3CA, PIK3CB, PIK3CD, PIK3CG, PIK3R1, PRKDC, SLC6A2, SLC6A3, SLC6A4 and at least one pharmaceutically acceptable excipient.
14. An anti-aging pharmaceutical composition comprising a compound selected from the group consisting of all compounds listed in the Table 1 ab and Table 1c and an enhancer moiety.
15. The composition of item 14, wherein the enhancer moiety is a permeability enhancer, stability enhancer or bioavailability enhancer.
16. A composition of any one of the items 14 or 15; further comprising a carrier.
17. A kit, comprising:
   a pharmaceutical composition comprising:
   an agent configured to bind to, inhibit, or degrade a protein selected from the group consisting of: Targets, wherein the agent is selected from the group consisting of a protein, a polymer, an aptamer, a SOMAmer, a peptide, a virus, a small molecule, a nanoparticle, an antibody, a monoclonal antibody, a polyclonal antibody, a humanized monoclonal antibody, a human monoclonal antibody, a human or humanized polyclonal antibody, and
   at least one pharmaceutically acceptable excipient; and
   an instruction for using the pharmaceutical composition as part of the anti-aging treatment or to treat or prevent an age-related disease or disorder.
18. Use of the compound selected from the group consisting of all compounds listed in the Table 1 ab and Table 1c for treating or preventing aging, frailty, an age-related disease or condition in a subject, for delaying or reversing one or more signs or symptoms of aging in a subject and/or for increasing longevity in a subject.
19. Use of the compound selected from the group consisting of all compounds listed in the Table 1 ab and Table 1c for treating or preventing a senescence associated disease or disorder in a subject and/or for selectively killing one or more senescent cells in a subject.
20. Use of the compound selected from the group consisting of all compounds listed in the Table 1 ab and Table 1c for treating or preventing viral disease, optionally COVID-19 or influenza, or infectious disease, or pneumonia in a subject or for alleviating signs and symptoms of viral disease, optionally COVID-19 or influenza, or infectious disease, or pneumonia in a subject.
21. The method, use, compound, composition or kit of any one of preceding items, wherein an age-related disease or condition is cancer.
22. Use of the compound selected from the group consisting of all compounds listed in the Table 1 ab and Table 1c for treating or preventing aging, frailty, an age-related disease or condition or for delaying or reversing one or more signs or symptoms of aging in a subject and/or for increasing longevity in a subject, wherein the aging, frailty, an age-related disease or condition is selected from the group consisting of age-related tissue decline, age-related organ decline, degenerative disease, function-decreasing disorder, Alzheimer's disease, Parkinson's disease, cataracts, macular degeneration, glaucoma, atherosclerosis, acute coronary syndrome, myocardial infarction, stroke, recovery after stroke, PSD-95 decrease, hypertension, idiopathic pulmonary fibrosis (IPF), chronic obstructive pulmonary disease (COPD), osteoarthritis, type 1 diabetes, obesity, fat dysfunction, coronary artery disease, cerebrovascular disease, periodontal disease, cancer treatment-related disability, chemotherapy treatment-related disability, chemotherapy treatment-related frailty, frailty, radiotherapy and other radiation related disability, chemotherapy treatment-related frailty, cancer treatment-related atrophy, cancer treatment-related fibrosis, brain injury, heart injury, and therapy-related myelodysplastic syndrome, accelerated aging, accelerated aging disease, Hutchinson-Gilford progeria syndrome, Werner syndrome, Cockayne syndrome, exroderma pigmentosum, ataxia telangiectasia, Fanconi anemia, dyskeratosis congenital, aplastic anemia, idiopathic pulmonary fibrosis, atherosclerosis, cardiovascular disease, adult cancer, arthritis, cataracts, osteoporosis, type 2 diabetes, diabetes, hypertension, neurodegeneration, stroke, atrophic gastritis, osteoarthritis, NASH, camptocormia, chronic obstructive pulmonary disease, coronary artery disease, dopamine dysregulation syndrome, metabolic syndrome, effort incontinence, Hashimoto's thyroiditis, heart failure, late life depression, immunosenescence, myocardial infarction, acute coronary syndrome, sarcopenia, sarcopenic obesity, senile osteoporosis and urinary incontinence.
23. The use of any one of preceding items, wherein the subject is a mammal.
24. The use of item 23, wherein the subject is a human, optionally a human of at least 60, 65, 70, 75, 80, 85, 90, 95 or 100 years of age.
25. The compound selected from the group consisting of all compounds listed in the Table 1 ab and Table 1c for use as a medicament.
26. The compound selected from the group consisting of all compounds listed in the Table 1 ab and Table 1c for use as a therapy.
27. Composition, comprising the compound selected from Table 1 ab and Table 1c and at least one pharmaceutically acceptable excipient for use as a medicament.
28. The compound selected from the group consisting of all compounds listed in the Table 1 ab and Table 1c for use as a food supplement.
29. A method for treating a senescence associated disease or disorder in a subject comprising administering to the subject a the compound selected from the group consisting of all compounds listed in table 1 ab and Table 1c or combination, comprising such compound; wherein the senescence associated disease or disorder is not a cancer, and wherein the such compound or combination is administered during a treatment course of 1-7 days every 0.5-12 months; provided that if the senescence associated disease or disorder is a senescence associated metabolic disorder, the senolytic combination is administered during a treatment course of 1-7 days every 4-12 months.
30. The method, use, compound, composition or kit of any one of preceding items, wherein such compound or combination is administered once every 0.5-12 months;
31. The method, use, compound, composition or kit of any one of preceding items, provided that if the senescence associated disease or disorder is a senescence associated metabolic disorder, the such compound or combination is administered once every 4-12 months.
32. The method, use, compound, composition or kit of any one of preceding items, wherein the age related disease or the senescent cell-associated disease or disorder is a cardiovascular disease or disorder, inflammatory disease or disorder, a pulmonary disease or disorder, a neurological disease or disorder.
33. The method, use, compound, composition or kit of any one of preceding items, wherein the cardiovascular disease or disorder is atherosclerosis.
34. The method, use, compound, composition or kit of any one of preceding items, wherein the inflammatory disease or disorder is osteoarthritis.
35. The method, use, compound, composition or kit of any one of preceding items, wherein the pulmonary disease or disorder is idiopathic pulmonary fibrosis or chronic obstructive pulmonary disease.
36. The method, use, compound, composition or kit of any one of preceding items, wherein the neurological disease or disorder is selected from mild cognitive impairment; motor neuron dysfunction; Alzheimer's disease; Parkinson's disease; and macular degeneration.
37. The method, use, compound, composition or kit of any one of preceding items, wherein the senescence associated metabolic disease or disorder is selected from diabetes, metabolic syndrome, and obesity.
38. The method, use, compound, composition or kit of any one of preceding items, wherein the senescence-associated disease or disorder is a dermatological disease or disorder is selected from eczema, psoriasis, hyperpigmentation, nevi, rashes, atopic dermatitis, urticaria, diseases and disorders related to photosensitivity or photoaging, rhytides; pruritis; dysesthesia; eczematous eruptions; eosinophilic dermatosis; reactive neutrophilic dermatosis; pemphigus; pemphigoid; immunobullous dermatosis; fibrohistocytic proliferations of skin; cutaneous lymphomas; and cutaneous lupus.
39. The method, use, compound, composition or kit of any one of preceding items, comprising administering to the subject the compound selected from the group consisting of all compounds listed in table 1 ab and Table 1c or combination, comprising such compound, wherein the senolytic combination is administered during a treatment course of 1-7 days every 4-12 months, and wherein the metabolic disease or disorder is selected from diabetes, metabolic syndrome, and obesity.
40. A method for treating a senescence-associated metabolic disease or disorder in a subject comprising administering to the subject the compound selected from the group consisting of all compounds listed in table 1 ab and Table 1c or combination, comprising such compound, wherein the senolytic combination is administered during a treatment course of 1-7 days every 4-12 months, and wherein the metabolic disease or disorder is selected from diabetes, metabolic syndrome, and obesity.
41. The method, use, compound, composition or kit of any one of preceding items, wherein the such compound or combination is administered once every 4-12 months.
42. The method, use, compound, composition or kit of any one of preceding items, wherein the senescent cell is selected from a senescent fibroblast, a senescent pre-adipocyte, a senescent epithelial cell, a senescent chondrocyte, a senescent neuron, and a senescent endothelial cell.
43. The method, use, compound, composition or kit of any one of preceding items, wherein the senescent cell is a senescent pre-adipocyte
44. The method, use, compound, composition or kit of any one of preceding items, comprising administering in or around an eye of the subject a pharmaceutical composition that contains an effective amount of a compound selected from the table 1 ab and Table 1c or a salt thereof.
45. A method of treating an ophthalmic disease or disorder that is not a cancer in a subject, the method comprising administering in or around an eye of the subject a pharmaceutical composition that contains an effective amount of a compound selected from the table 1 ab and Table 1c or a salt thereof.
46. The method of any one of preceding items, which is a method of treating age-related macular degeneration (AMD).
47. The method of any one of preceding items, which is a method of treating glaucoma.
48. The method of any one of preceding items, which is a method for preventing or delaying vision loss.
49. The method, use, compound, composition or kit of any one of preceding items, wherein the such method, use, composition or kit is for preventing or delaying vision loss.
50. The method, use, compound, composition or kit of any one of preceding items, wherein the composition is administered to the eye by intraocular injection.
51. The method, use, compound, composition or kit of any one of preceding items, wherein the composition is administered to the eye by intravitreal injection.
52. The method, use, compound, composition or kit of any one of preceding items, whereby p16 positive senescent cells in the eye that are causing one or more symptoms of the ophthalmic disease or disorder are removed from the eye.
53. The method, use, compound, composition or kit of any one of preceding items, wherein the pharmaceutical composition delays disorganization of type IV collagen in the eye.
54. The method, use, compound, composition or kit of any one of preceding items, wherein the pharmaceutical composition is administered in a therapeutically effective course of therapy that includes a period of treatment followed by a non-treatment interval of at least two weeks.
55. The method, use, compound, composition or kit of any one of preceding items, wherein administration of the pharmaceutical composition to the eye as a single dose is effective in decreasing severity or delaying progression of one or more symptoms of the ophthalmic disease for at least two weeks.
56. A method of treating an ophthalmic disease or disorder in a subject who does not have cancer, the method comprising administering intraocularly into an eye of the subject that is affected with the disease or disorder an effective amount of the compound selected from the table 1 ab and Table 1c, or a pharmaceutically acceptable salt thereof
57. A method of removing senescent cells from an eye of a subject in need thereof, wherein the senescent cells are identifiable as p16 positive cells that are not cancer cells, the method comprising contacting the senescent cells in the eye with an effective amount of compound selected from the table 1 ab and Table 1c, or a pharmaceutically acceptable salt thereof.
58. The method, use, compound, composition or kit of any one of preceding items, wherein the p16 positive senescent cells are causing or mediating one or more symptoms of an ophthalmic disease or disorder in the eye.
59. The method, use, compound, composition or kit of any one of preceding items, whereby one or more symptoms of an ophthalmic disease or disorder in the eye are decreased in severity or delayed in progression consequent to treating the eye to remove senescent cells.
60. The method, use, compound, composition or kit of any one of preceding items, further comprising determining whether positive senescent cells have been removed from the eye of the subject in which senescent cells have been contacted with said benzenesulfonamide
61. The method, use, compound, composition or kit of any one of preceding items, further comprising examining the subject to determine whether symptoms of an ophthalmic disease or disorder are decreased in severity or delayed in progression consequent to treating the eye to remove senescent cells.
62. A pharmaceutical composition, comprising the compound selected from the group consisting of all compounds listed in table 1 ab and Table 1c or their pharmaceutically acceptable salt or any combination of it or compounds or, optionally, having the similar SAR characteristics or any it's pharmaceutically acceptable salt, any it's hydrate, solvate, tautomer, geometric, optical and stereoisomer thereof, structurally related molecule, structural analog, functional analog, derivative, prodrug, or mixtures thereof in all ratios in therapeutically effective amount and at least one pharmaceutically acceptable excipient for anti-aging use.
63. The method, use, compound, composition or kit of any one of preceding items, comprising from 10 mg to 100 mg of compound selected from the table 1 ab and Table 1c as an active ingredient.
64. The method, use, compound, composition or kit of any one of preceding items, comprising from 1 mg to 50 mg of active ingredient.
65. The method, use, compound, composition or kit of any one of preceding items, comprising from 1 mg to 75 mg of active ingredient.
66. The method, use, compound, composition or kit of any one of preceding items, comprising from 1 mg to 100 mg of active ingredient.
67. The method, use, compound, composition or kit of any one of preceding items, comprising from 1 mg to 100 mg of active ingredient.
68. The method, use, compound, composition or kit of any one of preceding items, comprising from 1 mg to 25 mg of active ingredient.
69. The method, use, compound, composition or kit of any one of preceding items, comprising from 1 mg to 10 mg of active ingredient.
70. The method, use, compound, composition or kit of any one of preceding items, comprising from 1 mg to 5 mg of active ingredient.
71. The method, use, composition, pharmaceutical composition or kit of any one of preceding items, comprising from 1 mg to 5 g of active ingredient, wherein active ingredient is the compound selected from the group consisting of all compounds listed in table 1 ab and Table 1c or its pharmaceutically acceptable salt, any it's hydrate, solvate, tautomer, geometric, optical and stereoisomer thereof, structurally related molecule, structural analog, functional analog, derivative, prodrug, or mixtures thereof in all ratios in therapeutically effective amount and at least one pharmaceutically acceptable excipient.
72. The method, use, compound, composition or kit of any one of preceding items, comprising from 1 mg to 75 mg of active ingredient.
73. The method, use, compound, composition or kit of any one of preceding items, comprising from 10 mg to 75 mg of active ingredient.
74. The method, use, compound, composition or kit of any one of preceding items, comprising from 10 mg to 50 mg of active ingredient.
75. The method, use, compound, composition or kit of any one of preceding items, comprising from 10 mg to 25 mg of active ingredient.
76. The method, use, compound, composition or kit of any one of preceding items, comprising from 1 mg to 10 mg of active ingredient.
77. The method, use, compound, composition or kit of any one of preceding items, wherein composition is for oral administration.
78. The method, use, compound, composition or kit of any one of preceding items, wherein composition is for rectal administration.
79. The method, use, compound, composition or kit of any one of preceding items, wherein composition is for injection.
80. A use of pharmaceutical composition of any one of the preceding items for anti-aging treatment.
81. The method, use, compound, composition or kit of any one of preceding items, wherein pharmaceutical composition is for anti-aging treatment.
82. A use of the compound selected from the group consisting of all compounds listed in table 1 ab and Table 1c or any of their pharmaceutically acceptable salts, hydrates, solvates, tautomers, geometric, optical and stereoisomers thereof, structurally related molecules, structural analogs, functional analogs, derivatives, prodrugs, or mixtures thereof in all ratios in therapeutically effective amount for anti-aging treatment.
83. Kit, comprising pharmaceutical composition of any one of the preceding items and instruction for using it as an anti-aging treatment.
84. The method, use, compound, composition or kit of any one of preceding items, comprising the compound selected from the group consisting of all compounds listed in table 1 ab and Table 1c or any of their pharmaceutically acceptable salts, hydrates, solvates, tautomers, geometric, optical and stereoisomers thereof, structurally related molecules, structural analogs, functional analogs, derivatives, prodrugs, or mixtures thereof in all ratios in therapeutically effective amount and instruction for its use in anti-aging treatment.
85. A use of the compound selected from the group consisting of all compounds listed in table 1 ab and Table 1c or any of their pharmaceutically acceptable salts, hydrates, solvates, tautomers, geometric, optical and stereoisomers thereof, structurally related molecules, structural analogs, functional analogs, derivatives, prodrugs, or mixtures thereof in all ratios in therapeutically effective amount for manufacturing of anti-aging medication.
86. Method of anti-aging treatment, comprising administering to subject a therapeutically effective amount of the compound selected from the group consisting of all compounds listed in table 1 ab and Table 1c or any of their pharmaceutically acceptable salts, hydrates, solvates, tautomers, geometric, optical and stereoisomers thereof, structurally related molecules, structural analogs, functional analogs, derivatives, prodrugs, or mixtures thereof in all ratios in therapeutically effective amount or any combination of it.
87. The method, use, compound, composition or kit of any one of preceding items, wherein anti-aging treatment is a treatment or prevention an age-related disease or disorder or decline.
88. A method of selectively killing one or more senescent cells in a subject in need thereof, the method comprising administering to the subject a composition comprising the compound selected from the group consisting of all compounds listed in table 1 ab and Table 1c.
89. The method, use, compound, composition or kit of any one of preceding items, further comprising selectively killing one or more senescent cells in a subject in need thereof by the compound selected from the table 1 ab and Table 1c.
90. The method, use, compound, composition or kit of any one of preceding items, wherein the senescent cell is senescent due to replicative cellular senescence, premature cellular senescence, or therapy-induced senescence.
91. The method, use, compound, composition or kit of any one of preceding items, wherein the senescent cell is from an age-related pathology.
92. The method, use, compound, composition or kit of any one of preceding items, wherein the senescent cell is a therapy-induced senescent cell from normal and/or tumor tissue following DNA-damaging therapy.
93. The method, use, compound, composition or kit of any one of preceding items, wherein selectively killing senescent cells is assessed by the LD50 of the composition, wherein the LD50 of the composition in non-senescent cells is greater than 3 times higher than the LD50 of the composition in senescent cells.
94. The method, use, compound, composition or kit of any one of preceding items, wherein the killing is due to induction of apoptosis.
95. The method, use, compound, composition or kit of any one of preceding items, wherein the killing is measured as a reduction in viable cells.
96. The method, use, compound, composition or kit of any one of preceding items, wherein the reduction in viable cells is greater than 15%.
97. A method for delaying at least one feature or symptom of aging in a subject, the method comprising administering a composition comprising a therapeutically effective amount of the compound selected from the group consisting of all compounds listed in table 1 ab and Table 1c.
98. A method for anti-aging treatment, the method comprising administering a composition comprising a therapeutically effective amount of the compound selected from the group consisting of all compounds listed in table 1 ab and Table 1c.
99. A method for delaying at least one feature of aging in a subject, the method comprising administering a composition comprising a therapeutically effective amount of the compound selected from the group consisting of all compounds listed in table 1 ab and Table 1c.
100. The method, use, compound, composition or kit of any one of preceding items, wherein such method, use, compound, composition or kit for delaying at least one feature of aging in a subject.
101. The method, use, compound, composition or kit of any one of preceding items, wherein the subject has a received DNA-damaging therapy.
102. The method, use, compound, composition or kit of any one of preceding items, wherein the age-related disease or condition is a degenerative disease or a function-decreasing disorder.
103. The method, use, compound, composition or kit of any one of preceding items, wherein the age-related disease or condition is due to DNA-damaging therapy.
104. The method, use, compound, composition or kit of any one of preceding items, wherein the compound selected from the group consisting of all compounds listed in table 1 ab and Table 1c selectively kills therapy induced-senescent cells in normal and tumor tissues.
105. The method, use, compound, composition or kit of any one of preceding items, wherein the compound selected from the table 1 ab and Table 1c or composition, comprising such compound is administered in a dosage selected from the group consisting of: 10 times less than its LD50, 50 times less than its LD50, 100 times less than its LD50, 500 times less than its LD50, 1000 times less than its LD50, 5000 times less than its LD50, 10000 times less than its LD50, 2 times less than its MAXIMUM TOLERATED DOSE, 3 times less than its MAXIMUM TOLERATED DOSE, 4 times less than its MAXIMUM TOLERATED DOSE, 5 times less than its MAXIMUM TOLERATED DOSE, 7 times less than its MAXIMUM TOLERATED DOSE, 10 times less than its MAXIMUM TOLERATED DOSE, 50 times less than its MAXIMUM TOLERATED DOSE, 100 times less than its MAXIMUM TOLERATED DOSE, 500 times less than its MAXIMUM TOLERATED DOSE, 1000 times less than its MAXIMUM TOLERATED DOSE, 5000 times less than its MAXIMUM TOLERATED DOSE, 10000 times less than its MAXIMUM TOLERATED DOSE, Maximum Tolerated dose, 0.01 mg/mL, 0.05 mg/mL, 0.1 mg/mL, 0.5 mg/mL, 1 mg/mL, 5 mg/mL, 10 mg/mL, 50 mg/Ml, 100 mg/Ml, 500 mg/Ml, 1000 mg/Ml, in the dosage in which such compound was found effective in mice for at least one other indication known in the art, in the dosage in which such compound was found effective in human for at least one other indication known in the art.
106. A pharmaceutical composition of slow or controlled release providing a dosage according any one of preceding items.
107. A drug delivery device, providing dosage according any one of preceding items.
108. The method, use, compound, composition or kit of any one of preceding items, wherein composition is a compound selected from the table 1 ab and Table 1c.
109. The method, use, compound, composition or kit of any one of preceding items, comprising drug delivery device of any one of preceding items and instruction for using it as in anti-aging treatment.
110. The method, use, compound, composition or kit of any one of preceding items, wherein compound selected from the table 1 ab and Table 1c is selected from the group consisting of: CYCLOSPORINE, TARIQUIDAR, MARIMASTAT, PRINOMASTAT, APRATASTAT, YOHIMBINE, QUETIAPINE, DOXEPIN, MIANSERIN, ERGOTAMINE, PIPAMAZINE, PHENTOLAMINE, LISURIDE, TAMSULOSIN, DEXMEDETOMIDINE, INDORAMIN, RISPERIDONE, SERTINDOLE, XYLOMETAZOLINE, NAPHAZOLINE, TETRAHYDROZOLINE, DESIPRAMINE, ALFUZOSIN, SILODOSIN, TERAZOSIN, OLANZAPINE, ZOTEPINE, DROPERIDOL, THIORIDAZINE, CHLORPROMAZINE, FLUPHENAZINE, CARVEDILOL, OXYMETAZOLINE, HALOPERIDOL, PHENOXYBENZAMINE, NAFTOPIDIL, DOXAZOSIN, BROMOCRIPTINE, CLOZAPINE, PRAZOSIN, DIHYDROERGOTAMINE, CLOMIPRAMINE, AMITRIPTYLINE, IMIPRAMINE, NORTRIPTYLINE, ASTEMIZOLE, PROMAZINE, ZIPRASIDONE, PHENYLEPHRINE, CISAPRIDE, DAPIPRAZOLE, ILOPERIDONE, MORPHINE, ZOLMITRIPTAN, LOFEXIDINE, FIPAMEZOLE, FLUOXETINE, ATIPAMEZOLE, APRACLONIDINE, LEVONORDEFRIN, IDAZOXAN, BRIMONIDINE, GUANABENZ, EPINEPHRINE, NOREPINEPHRINE, CLONIDINE, CLONIDINE, METERGOLINE, PROCHLORPERAZINE, LY-2811376, LANABECESTAT, AZD-3839, VERUBECESTAT, BIRABRESIB, MIVEBRESIB, VORINOSTAT, ALOBRESIB, AZD-5153, PANOBINOSTAT, ACETAZOLAMIDE, BRINZOLAMIDE, MAFENIDE, INDISULAM, INDAPAMIDE, DTP-348 TOPIRAMATE, LEVOSULPIRIDE, DORZOLAMIDE, SULPIRIDE, CHLORTHALIDONE, METHAZOLAMIDE, NILOTINIB, ETHOXZOLAMIDE, ZONISAMIDE, DICHLORPHENAMIDE, SULTHIAME, METOLAZONE, TRICHLORMETHIAZIDE, SACCHARIN, LUTEOLIN, ISOQUERCETIN, DAIDZEIN, FAMOTIDINE, AZD-5438, TRILACICLIB, RGB-286638, ZOTIRACICLIB, LEROCICLIB, PHA-793887, PALBOCICLIB, HEXAMETHYL PARAROSANILINE, RG-547 DINACICLIB, FORETINIB, AST-487, AT-7519, AZD-7762, UCN-01, SUNITINIB, SILMITASERTIB, LESTAURTINIB, EDICOTINIB, ILORASERTIB, PEXIDARTINIB, TAK-593, PAZOPANIB, TANDUTINIB, DOVITINIB, CEP-32496, QUIZARTINIB, LINIFANIB, MOTESANIB, MASITINIB, DASATINIB, IMATINIB, SU-014813, RELACATIB, BALICATIB, ODANACATIB, ROTIGOTINE, CLEBOPRIDE, RACLOPRIDE, SUMANIROLE, PRAMIPEXOLE, PF-00217830, PALIPERIDONE, BENPERIDOL, BREXPIPRAZOLE, BIFEPRUNOX, HALOPERIDOL, DECANOATE, ARMODAFINIL, MODAFINIL, MESORIDAZINE, ASENAPINE, BLONANSERIN, CLOTHIAPINE, FALLYPRIDE, ROPINIROLE, SARIZOTAN, PERPHENAZINE, DOPAMINE, APOMORPHINE, PIMOZIDE, ARIPIPRAZOLE, RITANSERIN, CHLORPROTHIXENE, E-FLUPENTIXOL, TRIFLUOPERAZINE, DOMPERIDONE, Adoprazine, AMISULPRIDE, PERGOLIDE, CARIPRAZINE, FLUPENTIXOL, DRONABINOL, CLEMASTINE, KETANSERIN, LOXAPINE, ECOPIPAM, RALOXIFENE, TAMOXIFEN, CLOMIPHENE, ENCLOMIPHENE, IFENPRODIL, TRIPARANOL, TESEVATINIB, ROCILETINIB, CP-724714, AEE-788, CUDC-101 FALNIDAMOL, AZD-3759, DOCETAXEL, SAPITINIB, NAQUOTINIB, MAVELERTINIB PF-06459988 NAZARTINIB, OLMUTINIB, DACOMITINIB, GEFITINIB, ICOTINIB, NERATINIB, TAK-285 MIDOSTAURIN, AFATINIB, VANDETANIB, LAPATINIB, ERLOTINIB, CANERTINIB, IBRUTINIB, POZIOTINIB, VARLITINIB, OSIMERTINIB, PELITINIB, SORAFENIB, BOSUTINIB, PF-04457845, MK-3168 ORLISTAT, URB-597, AT-9283, TAFETINIB, CEP-11981, LUCITANIB, KRN-633, MK-2461, TIVOZANIB, SEMAXANIB, AXITINIB, BRIVANIB, VATALANIB, CEDIRANIB, BRIGATINIB, CEP-5214, BEMCENTINIB, CABOZANTINIB, CRENOLANIB, PACRITINIB, PONATINIB, BARASERTIB, ENMD-2076, CEP-1347, R-406, TOZASERTIB, KW-2449, FEDRATINIB, NINTEDANIB, GILTERITINIB, TELATINIB, LENVATINIB, CHLORAMBUCIL, FIMEPINOSTAT, QUISINOSTAT, ABEXINOSTAT, Givinostat, NANATINOSTAT, PYROXAMIDE, ROMIDEPSIN, MOCETINOSTAT, ENTINOSTAT, TRICHOSTATIN, BELINOSTAT, R-306465, BENDAMUSTINE, RICOLINOSTAT, CITARINOSTAT, AR-42, MAPROTILINE, PYRILAMINE, METHAPYRILENE, AZELASTINE, MIRTAZAPINE, TRIPROLIDINE, GSK-1004723, MIZOLASTINE, RUPATADINE, AZATADINE, CYPROHEPTADINE, HYDROXYZINE, CINNARIZINE, DESLORATADINE, CYCLIZINE, EBASTINE, BENZTROPINE, DIMETHINDENE, LEVOCETIRIZINE, CETIRIZINE, MEPAZINE, TERFENADINE, PROMETHAZINE, DEXCHLORPHENIRAMINE, CHLORPHENIRAMINE, AMOXAPINE, KETOTIFEN, PU-H71, TANESPIMYCIN, LUMINESPIB, GANETESPIB, BIIB021, ALVESPIMYCIN, ALVESPIMYCIN, GELDANAMYCIN, TRAZODONE, PRUVANSERIN, NELOTANSERIN, VOLINANSERIN, TEGASEROD, VELUSETRAG, TEMANOGREL, SEROTONIN, LYSERGIDE, METHYSERGIDE, ERGONOVINE, NEFAZODONE, METHYLERGONOVINE, OXITRIPTAN, LORCASERIN, CABERGOLINE, SUMATRIPTAN, CHLOROPHENYLPIPERAZINE, VABICASERIN, IDALOPIRDINE, CERLAPIRDINE, INTEPIRDINE, LANDIPIRDINE, LATREPIRDINE, JNJ-18038683, BMS-754807, CERITINIB, XL-228 LINSITINIB, AS-602868, DOFETILIDE, IBUTILIDE, HALOFANTRINE, VERAPAMIL, ANAGLIPTIN, CITALOPRAM, QUINIDINE, ESCITALOPRAM, QUININE, OSI-632, OSI-930, RIVOCERANIB, RG-1530, AG-13958, SITRAVATINIB, ALTIRATINIB, REGORAFENIB, CRIZOTINIB, SIROLIMUS, SARACATINIB, BAFETINIB, TALMAPIMOD, NEFLAMAPIMOD, QUERCETIN, CI-1040, DORAMAPIMOD, ARRY-797, PH-797804, VX-702, LOSMAPIMOD, TAK-715, PAMAPIMOD, R-1487, JNJ-49095397, CTS-1027, ZOTAROLIMUS, BGT-226, RG-7603 TACROLIMUS, EVEROLIMUS, PKI-179, AZD-8055, DACTOLISIB, GEDATOLISIB, OMIPALISIB, VISTUSERTIB, PF-04691502, APITOLISIB, SAPANISERTIB, BORTEZOMIB, IXAZOMIB, PD-0166285, RAF-265, TG100-115, TASELISIB, PICTILISIB, BUPARLISIB, COPANLISIB, ALPELISIB, BIMIRALISIB, AZD-8835, AZD-6482, GSK-2636771, NEMIRALISIB, GS-9901, LENIOLISIB, IDELALISIB, DUVELISIB, IPI-549, TAK-960, ONVANSERTIB, RIGOSERTIB, VOLASERTIB, GSK-461364, BI-2536 ADAVOSERTIB, LINOLEIC ACID, PEMAFIBRATE, NAMODENOSON, GW501516, INT131, EFATUTAZONE, MK-0533, ROSIGLITAZONE, MURAGLITAZAR, FARGLITAZAR, SEMAGACESTAT, BEGACESTAT, AVAGACESTAT, NIROGACESTAT, LIAFENSINE, NOMIFENSINE, 1-(3,4-DICHLOROPHENYL)-6-(METHOXYMETHYL)-3-AZABICYCLO[4.1.0]HEPTANE (ENANTIOMERIC MIX), ESREBOXETINE, REBOXETINE, DULOXETINE, ATOMOXETINE, MAZINDOL, AMPHETAMINE, DEXTROAMPHETAMINE, MILNACIPRAN, LEVOMILNACIPRAN, PROTRIPTYLINE, IOFLUPANE, FLUVOXAMINE, PYROVALERONE, VANOXERINE, PAROXETINE, LITOXETINE, AMITIFADINE, UK-390957 INDALPINE, VORTIOXETINE, VILAZODONE, MIDOMAFETAMINE, PRIMAQUINE, COCAINE, DEXTROMETHORPHAN, SERTRALINE, VENLAFAXINE, OZAGREL, TERBOGREL, DOLASTATIN-10, PATUPILONE.
111. The method, use, compound, composition or kit of any one of preceding items, wherein a dosage of compound selected from the table 1 ab and Table 1c is ranging from about 0.05 μg/kg to about 100 mg/kg of a patient's body weight, or 0.01 to about 1000 mg/kg of total body weight per day, or from about 0.1 to about 100 mg/kg of total body weight per day, or from about 0.5 to about 15 mg/kg of total body weight per day, or from about 1 mg/kg to about 50 mg/kg.
112. The method, use, compound, composition or kit of any one of preceding items, is for treatment of the disease, condition or disorder selected from the group, consisting of all diseases, conditions and disorders mentioned in this application.
113. The method, use, compound, composition or kit of any one of preceding items, wherein Compound selected from the table 1 ab and Table 1c is alleviating at least one of the symptoms of any one of the diseases, disorders and conditions selected from the group consisting of all diseases, disorders and conditions named in this application.
114. The method, use, compound, composition or kit of any one of preceding items, wherein Compound selected from the table 1 ab and Table 1c is in therapeutically effective amount.
115. The method, use, compound, composition or kit of any one of preceding items, wherein Compound selected from the table 1 ab and Table 1c is administered by the protocol selected from this application.
116. The method, use, compound, composition or kit of any one of preceding items, wherein Compound selected from the table 1 ab and Table 1c is for rejuvenation.
117. The method, use, compound, composition or kit of any one of preceding items, wherein the age-related disease or disorder or other disease or condition is associated with an alleviated level of a protein selected from the group consisting of Targets in a bodily fluid of the subject.
118. A method of providing an anti-aging treatment or of treating or preventing an age-related disease or disorder of a subject comprising reducing, inhibiting, or degrading a protein selected from the group consisting of Targets.
119. The method, use, compound, composition or kit of any one of preceding items, wherein treatment comprises reducing, inhibiting, or degrading a protein selected from the group consisting of Targets.
120. An anti-aging pharmaceutical composition, comprising a human cell, modified by the compound selected from the group consisting of all compounds listed in the Table 1 ab and Table 1c.
121. A human cell, modified by the compound selected from the group consisting of all compounds listed in the Table 1 ab and Table 1c.
122. A pharmaceutical composition, comprising a human cell, modified by the compound selected from the group consisting of all compounds listed in the Table 1 ab and Table 1c, wherein such composition is for treatment of disease, disorder or condition selected from this application.
123. The method, use, compound, composition or kit of any one of preceding items, wherein instead of compound selected from the table 1 ab and Table 1c a pharmaceutical composition, comprising a human cell, modified by the compound selected from the group consisting of all compounds listed in the Table 1 ab and Table 1c is used. The non-limiting example of such cell is hepatocyte, modified by MIZOLASTINE. Another non-limiting example of such cell is thrombocyte, modified by RUPATADINE. Yet another non-limiting example of such cell is brain neuron modified by TRIPROLIDINE. Yet another non-limiting example of such cell is Adipocyte, modified by RUPATADINE. In some of these examples, the compound selected from the table 1 ab and Table 1c is in therapeutically effective amount.
124. The method, use, compound, composition or kit of any one of preceding items, wherein instead of compound selected from the table 1 ab and Table 1c A pharmaceutical composition, comprising a human senescent cell, modified by the compound selected from the group consisting of all compounds listed in the Table 1 ab and Table 1c is used.
125. The method, use, compound, composition or kit of any one of preceding items, wherein instead of compound selected from the table 1 ab and Table 1c a human senescent cell, modified by compound selected from the table 1 ab and Table 1c is used.
   The non-limiting example of such cell is senescent cell, modified by MIZOLASTINE. Another non-limiting example of such cell is senescent cell, modified by RUPATADINE. Yet another non-limiting example of such cell is senescent cell modified by TRIPROLIDINE. Yet another non-limiting example of such cell is senescent cell, modified by any one of the compound selected from the table 1 ab and Table 1c. In some of these examples, the compound selected from the table 1 ab and Table 1c is in therapeutically effective amount.
      The compounds selected from the table 1 ab and Table 1c can be synthesised by the methods known in the art. Some of the compounds selected from the table 1 ab and Table 1c which chemical names are explicitly indicated can be readily obtained from commercial providers. Some examples of the providers: eMolecules, Inc, Bio-Techne Corporation (NASDAQ: TECH), Mcule, Enamine, ChemDiv, VitasM, UORSY, ChemBridge, LifeChemicals, ZelinskyInstitute, Specs, ChemicalBlock and Maybridge.

### DRAWINGS

Some embodiments are described below in relation to the drawings in which:

Figures are depicting prophetic examples for some of the embodiments, the real experiments can vary in in terms of the power of effect.

FIG. 1

FIG. 2 shows mortality delay in mice treated with the compound selected from Table 1 ab and Table 1c vs control animals. “Antibody” is a small molecule compound selected from the table 1 ab and Table 1c.

FIG. 3 shows the effect in mice of the experimental treatment with the compound selected from Table 1 ab and Table 1c on the Frailty Index (12 and 14 weeks after the intervention). “Antibody” is a small molecule compound selected from the table 1 ab and Table 1c.

FIG. 4 shows the effects of the experimental treatment in mice with the compound selected from Table 1 ab and Table 1c on the Open Field test results (at the baseline, 4 and 7 weeks after the intervention). “AB3” is a compound selected from the table 1 ab and Table 1c.

FIG. 5 shows the effects of the experimental treatments in mice with the compound selected from Table 1 ab and Table 1c on the markers of senescence in peripheral lymphocytes (16 and 18 weeks after the intervention). “AB3” is a compound selected from the table 1 ab and Table 1c.

FIG. 6 shows the effects of the experimental treatments with the compound selected from Table 1 ab and Table 1c on synaptoplasticity in mice (16 and 18 weeks after the intervention). “AB3” is a compound selected from the table 1 ab and Table 1c.

FIG. 7

FIG. 8

FIG. 14 shows the correlation of dFI (dynamic frailty index) with age. dFI is defined in Avchaciov, K et al. (2020).

FIG. 15 shows that dFI is associated with the number of senescent cells. Total flux (TF) in log scale representing p16-dependent luciferase reporter activity as a quantitative indicator of senescent cells: statistically significant correlations with age (a) and with dFI (b) in old mice (>50 weeks).

FIG. 16 shows that dFI correlates with Physiological Fraility Index.

FIG. 17 shows the predicted change in dFI under the treatment by the compound selected from Table 1 ab and Table 1c.

### DESCRIPTION OF VARIOUS EMBODIMENTS

Unless otherwise defined, scientific and technical terms used in connection with the present application shall have the meanings that are commonly understood by those of ordinary skill in the art. Further, unless otherwise required by context, singular terms shall include pluralities and plural terms shall include the singular. For example, the term “a cell” includes a single cell as well as a plurality or population of cells.

Terms of degree such as “about”, “substantially”, and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used herein, the terms “subject” and “animal” include all members of the animal kingdom. More specifically, the subject can be a vertebrate, e.g., a mammal such as a mouse, a primate, a simian or a human. Animals include, but are not limited to, farm animals, sport animals, and pets. A subject can be a healthy individual, an individual that has or is suspected of having a disease or a predisposition to the disease, or an individual that is in need of therapy or suspected of needing therapy, or an aged or frail individual. In one embodiment, the subject is a mammal. In a further embodiment, the subject is a human being.

The term “a cell” includes a single cell as well as a plurality or population of cells. Cells contemplated with the present application include microbial cells such as bacterial or yeast cells and mammalian cells.

The term “amino acid” as used herein refers to a compound having the following chemical structure:

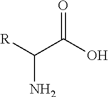

wherein “R” is known as the amino acid residue, or chemical functional group, that is present in the specified amino acid.

The term “natural amino acid” as used herein refers to an amino acid wherein R is one of the chemical functional groups present in amino acids occurring naturally in biological systems.

In an embodiment, the compound selected from Table 1 ab and Table 1c is useful for treatment or prevention of aging, frailty or an age-related disease or condition.

In an embodiment, the expression “the compound selected from Table 1 ab and Table 1c” as refers to an compound selected from compounds, which names or other identification or description are listed in Table 1 ab and Table 1c.

In an embodiment, the compound selected from Table 1 ab and Table 1c is modified for cell permeability, improved stability, and/or better bioavailability.

In some embodiments, the compound selected from Table 1 ab and Table 1c is also or additionally modified with an enhancer moiety. Accordingly, another aspect provides a compound comprising a compound selected from Table 1 ab and Table 1c and an enhancer moiety. In an embodiment, the compound selected from Table 1 ab and Table 1c is conjugated directly or indirectly to the enhancer moiety. As used herein, an enhancer moiety can increase or enhance the activity of the engineered compound. For example, the enhancer may be a permeability enhancer, a stability enhancer and/or a bioavailability enhancer. In an embodiment, the enhancer moiety is selected from a protein carrier, or a polymer carrier. In an embodiment, the enhancer moiety is a carrier protein, thereby forming a fusion protein. In another embodiment, the enhancer moiety is a PEG moiety.

In an embodiment, the compound selected from Table 1 ab and Table 1c is conjugated to a carrier protein, thereby forming a fusion protein.

In an embodiment, the compound selected from Table 1 ab and Table 1c of the application has at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as specified in any given compound selected from Table 1 ab and Table 1c, such compounds, in an embodiment, may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

In an embodiment, the compound selected from Table 1 ab and Table 1c and other compounds of the application are pharmaceutically acceptable salts. In an embodiment the pharmaceutically acceptable salt is an acid addition salt or a base addition salt. The selection of a suitable salt may be made by a person skilled in the art (see, for example, S. M. Berge, et al., “Pharmaceutical Salts,” J. Pharm. Sci. 1977, 66, 1-19). The term “pharmaceutically acceptable” as used herein means suitable for, or compatible with, the treatment of subjects.

An acid addition salt suitable for, or compatible with, the treatment of subjects is any non-toxic organic or inorganic acid addition salt. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In an embodiment, the mono- or di-acid salts are formed, and such salts exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. The selection of the appropriate salt may be useful, for example, so that an ester functionality, if any, elsewhere in a compound is not hydrolyzed. The selection criteria for the appropriate salt will be known to one skilled in the art.

Salts of the compounds of the application are generally formed by dissolving the neutral compound in an inert organic solvent and adding either the desired acid or base and isolating the resulting salt by either filtration or other known means.

Solvates of compounds of the application include, for example, those made with solvents that are pharmaceutically acceptable. Examples of such solvents include water (resulting solvate is called a hydrate) and ethanol and the like. The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

In an embodiment, the compounds of the present application further exist in varying polymorphic forms and it is contemplated that any polymorphs, or mixtures thereof, are included within the scope of the present application

### Compositions

The application also includes a composition, optionally a pharmaceutical composition, comprising the compound selected from Table 1 ab and Table 1c or other compounds described herein.

In an embodiment, the composition comprises a carrier or diluent.

In an embodiment, the carrier is a pharmaceutically acceptable carrier.

As used herein, the term “pharmaceutically acceptable carrier” is intended to include any and all solvents, dispersion media, coatings, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Optional examples of such carriers or diluents include, but are not limited to, water, saline, ringer's solutions, dextrose solution, and 5% human serum albumin and bovine serum albumin (BSA).

A composition is formulated to be compatible with its intended route of administration. Examples of routes of administration include parenteral (e.g., intravenous, subcutaneous, intramuscular), intradermal, intraperitoneal, subcutaneous, intranasal, epidural, sublingual, intravaginal, rectal, by inhalation, topical intracerebral, oral, intranasal, buccal, rectal, or transdermal administration routes. For example, in some instances, the composition described herein is formulated for local administration. This may achieved, for example, by topical application, local infusion during surgery, by injection, by means of a catheter, by means of a suppository or enema, or by means of an implant, the implant being of a porous, non-porous, or gelatinous material, including membranes, such as sialastic membranes, or fibers. In some situations, the composition described herein may introduced into the central nervous system, circulatory system or gastrointestinal tract by any suitable route, including intraventricular, intrathecal injection, paraspinal injection, epidural injection, enema, and by injection adjacent to a peripheral nerve. Pulmonary administration can also be employed, e.g., by use of an inhaler or nebulizer, and formulation with an aerosolizing agent, or via perfusion in a fluorocarbon or synthetic pulmonary surfactant.

In an embodiment, oral or parenteral compositions are formulated in dosage unit form for ease of administration and uniformity of dosage. Dosage unit form as used herein refers to physically discrete units suited as unitary dosages for the subject to be treated; each unit containing a predetermined quantity of active ingredient calculated to produce the desired therapeutic effect in association with the required pharmaceutical carrier. The specification for the dosage unit forms are dictated by and directly dependent on the unique characteristics of the active ingredient and the particular therapeutic effect to be achieved, and the limitations inherent in the art of preparing such an active ingredient for the treatment of individuals.

In an embodiment, the carrier is selected on the basis of compatibility with the compound disclosed herein, and the release profile properties of the desired dosage form. Exemplary carriers include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pennsylvania 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins 1999).

In an embodiment, the carrier protects the compound against rapid elimination from the body, such as in a sustained/controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such compositions will be apparent to those skilled in the art.

In an embodiment, the compositions further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In an embodiment, the compositions include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In an embodiment, the compositions include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In an embodiment, the compositions further include diluents which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®, dibasic calcium phosphate, dicalcium phosphate dihydrate, tricalcium phosphate, calcium phosphate, anhydrous lactose, spray-dried lactose, pregelatinized starch, compressible sugar, such as Di-Pac® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In an embodiment, the compositions include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In an embodiment, the compositions include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the compositions described herein for preventing, reducing or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, polysorbate-20 or Tween® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil, and polyoxyethylene alkylethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

In an embodiment, the compositions include, but are not limited to, aqueous fluid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

The amount of composition described herein that is effective for treating a corresponding disease or condition can be determined using standard clinical or pharmacokinetic techniques known to those with skill in the art. In addition, in vitro or in vivo assays can optionally be employed to help identify optimal dosage ranges. The precise dose to be employed can also depend on the route of administration, the disease or condition, the seriousness of the disease or condition being treated, as well as various physical factors related to the individual being treated, and can be decided according to the judgment of a health-care practitioner. For example, in an embodiment, the composition is a formulation for administration, in an amount of compound selected from the table 1 ab and Table 1c, binding agent, peptide, nucleic acid and/or recombinant cell ranging from about 0.05 μg/kg to about 100 mg/kg of a patient's body weight, or 0.01 to about 1000 mg/kg of total body weight per day, or from about 0.1 to about 100 mg/kg of total body weight per day, or from about 0.5 to about 15 mg/kg of total body weight per day, or from about 1 mg/kg to about 50 mg/kg of total body weight.

In an embodiment, the composition is formulated for administration in one or multiple doses per day or per week or per month or per 6 months or per year or per 3 years or per 8 years or per 12 years or once in a lifetime. In an embodiment, the composition is formulated for equivalent dosages which can be administered over various time periods including, but not limited to, about every 2 hours, about every 4 hours, about every 8 hours, about every 12 hours, about every 24 hours, about every 36 hours, about every 48 hours, about every 72 hours, about every week, about every two weeks, about every three weeks, about every month, and about every two months or every 6 months or every year or every 3 years or every 8 years or every 12 years or once in a lifetime or daily lifelong or as decided by practitioner or patient. The number and frequency of dosages corresponding to a completed course of therapy can be determined according to the judgment of a health-care practitioner. In an embodiment, in case of toxicity or adverse effects the administration is suspended or dosage decreased until the toxicity or adverse effects disappear and then the administration and/or dosage is resumed on the previous level.

In an embodiment, the dose for anti-aging use or any other use disclosed in this application is the same or about the same as an effective dose of the compound for its initial indication.

In an embodiment, the dose for anti-aging use or any other use disclosed in this application is the same or about the same as an effective dose of the compound for the indication for which such compound was approved. In an embodiment, the dose for anti-aging use or any other use disclosed in this application is the same or about the same as an effective dose of the compound for the indication for which such compound was tested in Phase 1 clinical trial or was effective in Phase 2 clinical trial.

### Kits

Also provided herein is a kit, comprising a compound selected from Table 1 ab and Table 1c or composition disclosed in this application and optionally a description or instruction for its use and additionally optionally further comprising medication labeling information.

In an embodiment, the instruction is an instruction for use of the compound selected from Table 1 ab and Table 1c or composition comprising such compound for treating or preventing aging, frailty or an age-related disease or condition in a subject, for delaying one or more signs or symptoms of aging in a subject and/or for increasing longevity in a subject. In an embodiment the instruction is an instruction for use of the compound selected from Table 1 ab and Table 1c or composition comprising such compound for treating or preventing a senescence associated disease or disorder in a subject and for selectively killing one or more senescent cells in a subject in need thereof. In other embodiments the instruction is an instruction for use of the compound selected from Table 1 ab and Table 1c or composition comprising such compound for treating or preventing viral disease, optionally COVID-19 or influenza, or infectious disease, or pneumonia in a subject or for alleviating signs and symptoms of viral disease, optionally COVID-19 or influenza, or infectious disease in a subject.

### Methods and Uses

The application also provides uses and methods relating to the compound selected from Table 1 ab and Table 1c or composition comprising such compound.

In an embodiment, the activity of the target, selected from the Targets tt can be inhibited by at least 5, 10, 15, 25, 50, 75 or 100%. Inhibiting includes any decrease in activity as compared to activity under otherwise the same conditions except in the absence of the binding agent.

### Methods of Treatment or Prevention

The compounds selected from Table 1 ab and Table 1c or compositions comprising such compounds are useful for treating or preventing a condition selected from the group consisting of: aging, frailty, an age-related disease or condition in a subject, for delaying one or more signs or symptoms of aging in a subject and/or for increasing longevity in a subject. The compounds selected from Table 1 ab and Table 1c or compositions comprising such compounds, compounds and compositions of the present application are also useful for treating or preventing a senescence associated disease or disorder in a subject and for selectively killing one or more senescent cells in a subject in need thereof.

In an embodiment, the compounds selected from Table 1 ab and Table 1c or compositions comprising such compounds are used in a method for treating or preventing aging, frailty, an age-related disease or condition in a subject, for delaying one or more signs or symptoms of aging in a subject, for increasing longevity in a subject, treating or preventing a senescence associated disease or disorder in a subject and/or for selectively killing one or more senescent cells in a subject, the method comprising administering an effective amount of the compound selected from Table 1 ab and Table 1c or composition comprising such compound to a subject in need thereof.

In an embodiment, a use of a compound selected from Table 1 ab and Table 1c or composition comprising such compound for treating or preventing aging, frailty or an age-related disease or condition in a subject, for reversing or delaying one or more signs or symptoms of aging in a subject, for increasing longevity in a subject, treating or preventing a senescence associated disease or disorder in a subject and/or for selectively killing one or more senescent cells in a subject is included. In another embodiment, a use of compound selected from Table 1 ab and Table 1c or composition comprising such compound for preparation of a medicament for treating or preventing aging, frailty or an age-related disease or condition in a subject, for delaying one or more signs or symptoms of aging in a subject, for increasing longevity in a subject, treating or preventing a senescence associated disease or disorder in a subject and/or for selectively killing one or more senescent cells in a subject is provided.

In another embodiment, a compound selected from Table 1 ab and Table 1c or composition comprising such compound is for use in treating or preventing aging, frailty or an age-related disease or condition in a subject, for delaying one or more signs or symptoms of aging in a subject, for increasing longevity in a subject, treating or preventing a senescence associated disease or disorder in a subject and/or for selectively killing one or more senescent cells in a subject is included.

In an embodiment, a compound selected from Table 1 ab and Table 1c or composition comprising such compound or other compound or composition disclosed herein is for use in preventing or slowing down the progression of senescence or some of its features. In an embodiment, a compound selected from Table 1 ab and Table 1c or composition comprising such compound, compound or composition disclosed herein for interfering with the progression of cells entering senescence or modulating their activity by reducing senescence-associated secretory phenotype (SASP) generation. In an embodiment, a compound selected from Table 1 ab and Table 1c or composition comprising such compound, or composition disclosed herein for use as senostatic.

As used herein, the phrase “treating” includes reversing, alleviating or inhibiting the progression of a disease or condition or symptoms or conditions associated the disease or condition. “Treating” also includes extending survival in a subject.

In some embodiments, the term “aging” refers to an age-dependent or age-progressive decline in intrinsic physiological function.

As used herein, in some embodiments, the expression “treating or preventing aging, frailty or an age-related disease or condition” relates to the amelioration at least one symptom of the age-related condition in the subject, amelioration at least one symptom of the age-related disease in the subject, amelioration or lessening of the effects of aging, decreasing or delaying an increase in the biological age, slowing rate of aging; treatment, prevention, amelioration and lessening the effects of frailty or at least one of aging related diseases and conditions or declines or slowing down the progression of such decline (including but not limited to those indicated in Table 4, “Declines”), condition or disease, increasing health span or lifespan, rejuvenation, increasing stress resistance or resilience, increasing rate or other enhancement of recovery after surgery, radiotherapy, disease and/or any other stress, decreasing all-causes or multiple causes of mortality risks or mortality risks related to at least one or at least two of age related diseases or conditions or delaying in increase of such risks, decreasing morbidity risks. In some embodiments, the treatment leading to the modulating of at least one of biomarkers of aging into more youthful state or slowing down its change into “elder” state is also regarded to be an anti-aging treatment, including but not limited to biomarkers of aging which are visible signs of aging, such as wrinkles, grey hairs etc.

In the embodiment, an age-related disease or condition is selected from: age-related tissue decline, age-related organ decline, degenerative disease, function-decreasing disorder, Alzheimer's disease, Parkinson's disease, cataracts, macular degeneration, glaucoma, atherosclerosis, acute coronary syndrome, myocardial infarction, stroke, recovery after stroke, PSD-95 decrease, hypertension, idiopathic pulmonary fibrosis (IPF), chronic obstructive pulmonary disease (COPD), osteoarthritis, type 2 diabetes, obesity, fat dysfunction, coronary artery disease, cerebrovascular disease, periodontal disease, cancer treatment-related disability, chemotherapy treatment-related disability, chemotherapy treatment-related frailty, frailty, radiotherapy and other radiation related disability, chemotherapy treatment-related frailty, cancer treatment-related atrophy, cancer treatment-related fibrosis, brain injury, heart injury, and therapy-related myelodysplastic syndrome, accelerated aging, accelerated aging disease, Hutchinson-Gilford progeria syndrome, Werner syndrome, Cockayne syndrome, exroderma pigmentosum, ataxia telangiectasia, Fanconi anemia, dyskeratosis congenital, aplastic anemia, idiopathic pulmonary fibrosis, atherosclerosis, cardiovascular disease, adult cancer, arthritis, cataracts, osteoporosis, type 2 diabetes, hypertension, neurodegeneration (including but not limited to Alzheimer's disease, Huntington's disease, and other age-progressive dementias; Parkinson's disease; and amyotrophic lateral sclerosis [ALS]), stroke, atrophic gastritis, osteoarthritis, NASH, camptocormia, chronic obstructive pulmonary disease, coronary artery disease, dopamine dysregulation syndrome, metabolic syndrome, effort incontinence, Hashimoto's thyroiditis, heart failure, late life depression, immunosenescence, myocardial infarction, acute coronary syndrome, sarcopenia, sarcopenic obesity, senile osteoporosis and urinary incontinence.

In an embodiment, “treating or preventing aging, frailty or an age-related disease or condition” includes cell tumorigenesis through autophagy-related cell death or autophagy induction. In an embodiment, “treating or preventing aging, frailty or an age-related disease or condition” comprises treatment of any one of the disease, condition or decline known in the art as treated by any one of anti-aging interventions, including but not limited to metformin, NAD+, melatonin, rapamycin, any rapalog, any senolytic, BASIS™, caloric restriction or 16/8 intermittent fasting.

Aging-related changes in any parameter or physiological metric are also regarded as age-related conditions, including but not limited to aging related change in blood parameters, heart rate, cognitive functions/decline, bone density, basal metabolic rate, systolic blood pressure, heel bone mineral density (BMD), heel quantitative ultrasound index (QUI), heel broadband ultrasound attenuation, heel broadband ultrasound attenuation, forced expiratory volume in 1-second (FEV1), forced vital capacity (FVC), peak expiratory flow (PEF), duration to first press of snap-button in each round, reaction time, mean time to correctly identify matches, hand grip strength (right and/or left), whole body fat-free mass, leg fat-free mass (right and/or left), and time for recovery after any stress (wound, operation, chemotherapy, disease, change in lifestyle etc.). Aging related change in any parameter of organism is also regarded as an aging related condition, including but not limited to aging related change in at least one of the parameter selected from the Table 4 “Declines”.

In some embodiments, the signs and symptoms of aging in mammals such as humans include, but are not limited to, hearing loss, cognitive decline, wrinkles, fertility decline, hair greying, osteoarthritis, frailty, atherosclerosis, generalized organ atrophy, diminished stress tolerance and reduced longevity. The aging process is also manifested at the cellular level. In some embodiments, signs and symptoms of cellular aging include, but are not limited to, loss of doubling capacity, increased levels of apoptosis, changes in differentiated phenotype, and changes in metabolism, e.g., decreased levels of protein synthesis and turnover. Telomere shortening may also be an indicator of aging. In an embodiment, any of the signs and symptoms described above are decreased by at least 5, 10, 25, 50, 75 or 100% compared to what would be expected without treatment as described herein.

The length of time from birth to death is known as the lifespan of an organism, and each organism has a characteristic average lifespan. In addition, cells which are not capable of continuous growth in culture (non-immortal cells or cell lines) are characterized by a predictable lifespan in vitro, broadly divisible into three phases corresponding to growth, maturation, and decline (i.e., senescence). The lifespans of many non-immortal cells in culture, particularly mammalian cells, frequently varies from only a matter of hours to only several weeks, even under optimal culture conditions. Even “immortal cells” tend to lose viability as a function of time in culture, with corresponding decline of the cell mass.

As used herein, the term “increasing longevity” includes extending lifespan. In an embodiment, lifespan is extended by at least 1, 3 or 5 days, 1, 2, 3, 4, 5 or 6 weeks, 1, 2, 3, 6 or 12 months, or at least 2, 3, 4, 5, 10, 15, 20, 25, 30 or 40 years over the lifespan that would be expected without treatment as described herein.

As used herein, an “FSTL3 associated disease or condition” is a disease or condition that has a relationship with the presence, absence, level and/or function of FSTL3 or a disease or condition that is characterized by the presence, absence, level and/or function of FSTL3. For example, Gao et al. (2020) have shown that up-regulation of FSTL3 promotes proliferation and migration of non-small cell lung cancer cells. In addition, in WO2018067754A1 it was shown that FSTL3 inhibition in particular embodiments, has one or more activities on pancreatic islet cells selected from the group consisting of increasing insulin secretion from beta cells, increasing beta cell regeneration, promoting transdifferentiation of an alpha cell or any other pancreatic cell to a beta cell, and inhibiting transdifferentiation of a beta cell to an alpha cell, or any combination thereof. It has also been shown that FSTL3 Neutralizing Antibodies Restore Function to Diabetic Mouse and Human Islets (Schneyer et al., 2020). In an embodiment, a compound selected from the table 1 ab and Table 1c or composition, comprising such compound is useful for treatment of FSTL3 associated disease or condition.

In an embodiment, an age-related disease or condition is cancer, optionally a cancer selected from: non-small cell lung cancer, atypical teratoid rhabdoid tumor, brain tumor, anal cancer, astrocytoma, vaginal cancer, extrahepatic bile duct cancer, intraocular melanoma, hairy cell leukemia, hepatocellular liver cancer, gestational trophoblastic disease, germ cell tumor, hypopharyngeal cancer, histiocytosis, histiocytosis Langerhans, high-grade astrocytoma, astrocytoma, glioma, brain stem glioma, invasive lobular carcinoma, gastrointestinal carcinoid tumor, gastrointestinal stromal tumor, cutaneous T-cell lymphoma, peripheral T-cell lymphoma, non-specific lymphoma mantle cell, lymphogranulomatosis, colorectal cancer, craniopharyngioma, leukemia, mast cell leukemia, Burkitt's lymphoma, Hodgkin's lymphoma, Waldenstrom's macroglobulinemia (lymphoplasmacytic lymphoma), small bowel cancer, mastocytosis, malignant mesothelioma, melanoma, small-cell carcinoma (small-cell lung cancer), metastatic squamous neck cancer, myelodysplastic/myeloproliferative neoplasms, myelodysplastic syndrome, acute myeloid leukemia, chronic myelogenous leukemia, chronic myeloproliferative disease, multiple myeloma (plasma cell myeloma or Kahler's disease), male breast cancer, nasal cell carcinoma, neuroblastoma, non-small cell lung cancer, non-Hodgkin's lymphoma, Wilms tumor, osteosarcoma, malignant fibrous histiocytoma of bone, acute lymphoblastic leukemia, acute myeloid leukemia, papillomatosis, paraganglioma, parathyroid carcinoma, transitional cell cancer of the renal pelvis, transitional cell cancer of the ureter, pleuropulmonary blastoma, squamous cell carcinoma, renal cell carcinoma, ductal carcinoma in situ, rhabdomyosarcoma, vulvar cancer, eye cancer, head and neck cancer, throat cancer, laryngeal cancer, lip and oral cancer, stomach cancer, gall bladder cancer, bile duct cancer, skin cancer, cancer of the adrenal cortex, bone cancer, uterine cancer, Merkel carcinoma, bladder cancer, nasopharyngeal cancer, esophageal cancer, penile cancer, nasal cavity cancer, paranasal sinus cancer, renal pelvis cancer, ureter cancer, renal cancer, Papillary renal cell carcinoma, prostate cancer, rectal cancer, oral cancer, salivary gland cancer, cancer of the urethra, cancer of the cervix, thyroid cancer, endometrial cancer, cancer of the central nervous system, testis cancer, ovarian cancer, retinoblastoma, sarcoma, Kaposi's sarcoma, uterine sarcoma, soft tissue sarcoma, Ewing's sarcoma, cardiac tumor, Sezary syndrome, pharyngeal cancer, pheochromocytoma, fibrous histiocytoma of bone, chordoma, chronic myeloproliferative disorder, chronic lymphocytic leukemia, ependymoma, erythroleukemia, esthesioneuroblastoma.

Thus, in another embodiment, the age-related disease or condition is diabetes, optionally type 1 diabetes or type 2 diabetes.

In yet another example, the age-related disease or condition is a cardiovascular disorder, a cognitive disorder, a neurodegenerative disorder, a metabolic disorder, a muscular disorder, cardiac hypertrophy, diastolic heart, left ventricle wall thickness, myocardial fibrosis, or both left ventricle wall thickness and myocardial fibrosis any condition set out in WO2016049662.

In another embodiment, the age-related disease or condition comprises at least one symptom of diastolic heart failure in a subject in need thereof. In certain embodiments, the subject has preserved ejection fraction but elevated left ventricular diastolic pressure (LVDP), as compared to subjects without diastolic heart failure. In certain embodiments, the subject has preserved ejection fraction but elevated myocardial fibrosis, as compared to subjects without diastolic heart failure. In another embodiment the age-related disease or condition comprises diastolic heart failure.

Cellular senescence is characterized by the cessation of cell division. As used herein, the expression “selectively killing one or more senescent cells refers to “selectively” (preferentially or to a greater degree) destroying, killing, removing, or facilitating selective destruction of senescent cells. In other words, the binding agent or composition destroys or kills a senescent cell in a biologically, clinically, and/or statistically significant manner compared with its capability to destroy or kill a non-senescent cell. In an embodiment, the binding agent or composition is used in an amount and for a time sufficient that selectively kills established senescent cells but is insufficient to kill (destroy, cause the death of) a non-senescent cell in a clinically significant or biologically significant manner. In an embodiment, alternately or in combination, the compound selected from the table 1 or composition, comprising such compound, selectively or differentially or disproportionately kills senescent cells over non-senescent cells when administered systemically or locally to a mammal. In an embodiment senescent cells are killed, for example by apoptosis. In an embodiment “selectively killing one or more senescent cells” is assessed, for example, by the LD50 of the compound selected from the table 1 or composition, comprising such compound, wherein the LD50 of the compound selected from the table 1 or composition, comprising such compound, in non-senescent cells is greater than 3 times than the LD50 of the composition in senescent cells.

In an embodiment, killing is measured as a reduction in viable cells. In an embodiment, the reduction in viable cells is greater than 15% following the methods and uses described herein.

In an embodiment, “a senescence associated disease or disorder in a subject” refers to a disease or disorder which is fully or partially mediated by the induction or maintenance of a non-proliferating or senescent state in a cell or a population of cells in a subject. Non-limiting examples of senescence associated diseases or disorders include cardiovascular diseases such as angina, aortic aneurysm, arrhythmia, brain aneurysm, cardiac diastolic dysfunction, cardiac fibrosis, cardiac stress resistance, cardiomyopathy, carotid artery disease, coronary thrombosis, endocarditis, hypercholesterolemia, hyperlipidemia, mitral valve prolapsed, and peripheral vascular disease; inflammatory or autoimmune diseases such as herniated intervertebral disc, inflammatory bowel disease, kyphosis, oral mucositis, lupus, interstital cystitis, scleroderma, and alopecia; neurodegenerative diseases such as dementia, Huntington's disease, motor neuron dysfunction, age-related memory decline, and depression/mood disorders; metabolic diseases such as diabetic ulcer and metabolic syndrome; pulmonary diseases such as age-related loss of pulmonary function, asthma, bronchiectasis, cystic fibrosis, emphysema, and age-associated sleep apnea; gastrointestinal diseases such as Barrett's esophagus; age-related disorders such as liver fibrosis, muscle fatigue, oral submucosa fibrosis, pancreatic fibrosis, benign prostatic hyperplasia (BPH), and age-related sleep disorders; reproductive disorders such as menopause (male and female), egg supply (female), sperm viability (male), fertility (male and female), sex drive, and erectile function and arousal (male and female); dermatological diseases such as atopic dermatitis, cutaneous lupus, cutaneous lymphomas, dysesthesia, eczema, eczematous eruptions, eosinophilic dermatosis, fibrohistocytic proliferations of skin, fibrosis, lung fibrosis, hyperpigmentation, immunobullous dermatosis, nevi, pemphigoid, pemphigus, pruritis, psoriasis, rashes, reactive neutrophilic dermatosis, rhytides, and urticarial; and other diseases such as diabetic wound healing, post-transplant kidney fibrosis, Arthritis (Osteo- and Rheumatoid), Cancer Therapy-Related Disability, Hypertension, Metabolic syndrome, Ocular Diseases (such as Glaucoma), Osteoporosis, Sarcopenia/Cachexia, Transplantation Rejection, Type 2 Diabetes, Yamamoto's Muscular Dystrophy, Alzheimer's Disease, Amyotrophic Lateral Sclerosis, Chronic Obstructive Pulmonary Disease, Huntington's Chorea, Idiopathic Pulmonary Fibrosis, Juvenile Rheumatoid Arthritis (JRA), NAFL, NASH, diet induced liver disease, alcohol induced liver disease, and carotid thrombosis.

Mortality from acute respiratory diseases including pneumonia, influenza and COVID-19 is strongly age dependent. Accordingly, a compound selected from the table 1 ab and Table 1c or composition, comprising such compound, other compounds and compositions of the present application are useful for treating or preventing viral disease, optionally COVID-19 or influenza, or infectious disease, or pneumonia in a subject or for alleviating signs and symptoms of viral disease, optionally COVID-19 or influenza, or infectious disease, or pneumonia in a subject.

Thus, in an embodiment, a compound selected from the table 1 ab and Table 1c or composition, comprising such compound, compounds and compositions described herein are used in a method for treating or preventing viral disease, optionally COVID-19 or influenza, or infectious disease, or pneumonia in a subject or for alleviating signs and symptoms of viral disease, optionally COVID-19 or influenza, or infectious disease, or pneumonia in a subject, the method comprising administering an effective amount of a compound selected from the table 1 ab and Table 1c or composition, comprising such compound, other compound or composition disclosed herein to a subject in need thereof.

In an embodiment, a use of a compound selected from the table 1 ab and Table 1c or composition, comprising such compound, other compound or composition disclosed herein for treating or preventing viral disease, optionally COVID-19 or influenza, or infectious disease, or pneumonia in a subject or for alleviating signs and symptoms of viral disease, optionally COVID-19 or influenza, or infectious disease, or pneumonia in a subject is included.

In an embodiment, a compound selected from the table 1 ab and Table 1c or composition, comprising such compound, other compound or composition disclosed herein for use in treating or preventing viral disease, optionally COVID-19 or influenza, or infectious disease, or pneumonia in a subject or for alleviating signs and symptoms of viral disease, optionally COVID-19 or influenza, or infectious disease, or pneumonia in a subject is included. In another embodiment, a use of a compound selected from the table 1 ab and Table 1c or composition, comprising such compound, other compound or composition disclosed herein for preparation of a medicament for treating or preventing viral disease, optionally COVID-19 or influenza, or infectious disease, or pneumonia in a subject or for alleviating signs and symptoms of viral disease, optionally COVID-19 or influenza, or infectious disease, or pneumonia in a subject is provided.

In another embodiment, a compound selected from the table 1 ab and Table 1c or composition, comprising such compound, other compound or composition disclosed herein is for use in treating or preventing viral disease, optionally COVID-19 or influenza, or infectious disease, or pneumonia in a subject or for alleviating signs and symptoms of viral disease, optionally COVID-19 or influenza, or infectious disease, or pneumonia in a subject.

In an embodiment, the viral disease is COVID-19, or a disease, caused by SARS-CoV, SARS-CoV-2 (Severe acute respiratory syndrome coronavirus 2) or other coronavirus. In an embodiment, a compound selected from the table 1 ab and Table 1c or composition, comprising such compound is used for reduction of pulmonary fibrosis caused by viral infection of SARS-CoV, reduction of pulmonary fibrosis caused by viral infection of SARS-CoV-2, reduction of virus titer of SARS-CoV and/or reduction of virus titer of SARS-CoV-2.

In another embodiment, a compound selected from the table 1 ab and Table 1c or composition, comprising such compound, other compound or composition and an immunotherapy are used in a method of treating a cancer or of an infectious disease in a subject in need thereof, the method comprising administering an effective amount of a compound selected from the table 1 ab and Table 1c or composition, comprising such compound, other compound or composition and an immunotherapy disclosed herein to a subject in need thereof. In yet another embodiment, any other immunotherapy is used.

In an embodiment, a use of a compound selected from the table 1 ab and Table 1c or composition, comprising such compound, other compound or composition disclosed herein and an immunotherapy for treating a cancer or an infectious disease is included. In another embodiment, a use of an A compound selected from the table 1 ab and Table 1c or composition, comprising such compound, other compound or composition disclosed herein and an immunotherapy for preparation of a medicament for treating a cancer or an infectious disease is provided. In another embodiment, a compound selected from the table 1 ab and Table 1c or composition, comprising such compound, other compound or composition disclosed herein and an immunotherapy is for treating a cancer or an infectious disease.

In some embodiments, the compound selected from the table 1 ab and Table 1c or composition, comprising such compound, other compound or composition disclosed herein is an adjuvant for the immunotherapy. In another embodiment, the compound selected from the table 1 ab and Table 1c or composition, comprising such compound, other compound or composition disclosed used as a conditioning regimen for the immunotherapy, a conditioning regimen being a therapy for preparing the subject for the immunotherapy.

In some embodiments, the cancer is a solid cancer selected from the group consisting of melanoma, breast carcinoma, colon carcinoma, renal carcinoma, adrenocortical carcinoma, testicular teratoma, skin sarcoma, fibrosarcoma, lung carcinoma, adenocarcinoma, liver carcinoma, glioblastoma, prostate carcinoma and pancreatic carcinoma or any other cancer described in this application. In another embodiment, the infectious disease caused by a virus, a bacterium, a fungus or a protozoan parasite.

The compound selected from the table 1 ab and Table 1c or composition, comprising such compound, other compound or composition disclosed herein can be used or administered prior to and/or concomitantly with the immunotherapy.

In one embodiment, said immunotherapy comprises an adoptive transfer of immune cells, for example, T cells, natural killer (NK) cells, CAR T cells, CAR NK cells, autologous immune cells or CD8+ T cells.

In another embodiment, said immunotherapy comprises a checkpoint inhibitor for example inhibitors of PD−1 such as pembrolizumab, nivolumab, cemiplimab, tislelizumab, spartalizumab, ABBV-181 and JNJ-63723283, inhibitors of PD-L1 such as avelumab, atezolizumab and durvalumab, inhibitors of CTLA-4 such as ipilimumab and tremelimumab, and any mixtures thereof.

In another embodiment, said immunotherapy comprises ipilimumab, Tremelimumab, AGEN1181, BMS-986218, BMS-986205, REGN4659, SDREGN2810, ADU-1604, Balstilimab, cemiplimab, nivolumab, pembrolizumab, avelumab, durvalumab, atezolizumab, Cemiplimab, BCD-217, BCD-100, BCD-145, Pembrolizumab, Vopratelimab, JTX-2011, MEDI4736, Camrelizumab (SHR-1210), Lirilumab, CS1002, CS1003, Relatlimab, AntiCD137, AK104, XmAb20717, NKTR-214 and any mixtures thereof.

In another embodiment, said immunotherapy comprises a vaccination.

In another embodiment, the immunotherapy is selected from the group consisting of: PD-1 inhibitor, PD-L1 inhibitor, T-Cell stimulant, CTLA antibody, T-cell surface glycoprotein, CD3 antibody, T-cell surface glycoprotein CD3 antibody, rituximab (Rituxan), Brentuximab Vedotin (Adcetriz), Ado-trastuzumab emtansine (Kadcyla) Cetuximab (Erbitux), bevacizumab (Avastin), Ibritumomab (Zevalin), vedolizumab (Entyvio), pembrolizumab (Keytruda), Alemtuzamab atezolizumab (Tecentriq), avelumab (Bavencio), durvalumab (Imfinzi), B-701, Ofatumumab, Obinutuzumab (Gazyva) Panitumumab, plozalizumab, BI-754091, OREG-103, COM-701, B 1-754111, Keytruda (MOA: PD-1), Opdivo (MOA: PD-1), Tecentriq (MOA: PD-L1), Imfinzi (MOA: PD-L1), Yervoy (MOA: CTLA), Yescarta (MOA: T-Cell), INCSHR1210 (MOA: PD-1), Tislelizumab (MOA: PD-1), ide-cel (MOA: T-Cell), Kymriah (MOA: T-Cell), Tislelizumab (MOA: PD-1), Atezolizumab (Tecentriq), Durvalumab (Imfinzi), Avelumab (Bavencio), Yescarta (axicabtagene ciloleucel), Kymriah (tisagenlecleucel), and combinations thereof.

In an embodiment, the subject is a subject who is an “aged subject”. An “aged subject” is understood as a human being of chronological age (or in some embodiments, of biological age) of 30 years or older, 35 years or older, 40 years or older, 45 years or older, 50 years or older, 55 years or older, 60 years or older, 65 years or older, 70 years or older, 75 years or older, 80 years or older, 85 years or older, 90 years or older, 95 years or older. In an embodiment of this application “aged subject” is understood as a frail subject.

In an embodiment, the compounds selected from the table 1 ab and Table 1c or compositions, comprising such compounds, other compounds and compositions described herein are for use parenterally (e.g., intravenously, subcutaneously, intramuscularly), intradermally, intraperitoneally, subcutaneously, intranasally, epidurally, sublingually, intravaginally, rectally, by inhalation, topical intracerebrally, orally, intranasally, buccally, or transdermally or are administered parenterally (e.g., intravenously, subcutaneously, intramuscularly), intradermally, intraperitoneally, subcutaneously, intranasally, epidurally, sublingually, intravaginally, rectally, by inhalation, topical intracerebrally, orally, intranasally, buccally, or transdermally. In another embodiment, the compounds selected from the table 1 ab and Table 1c or compositions, comprising such compounds are for use topically or are administered topically.

An effective amount of a compound selected from the table 1 ab and Table 1c or composition, comprising such compound, other compound or composition of the application relates generally to the amount needed to achieve a desired objective.

The amount required to be administered will furthermore depend on the activity of the compounds selected from the table 1 ab and Table 1c or compositions, comprising such compound will also depend on the rate at which an administered compound is depleted from the free volume of the subject to which it is administered. Common ranges for effective dosing of a compound of the application may be, by way of non-limiting example, from about 0.01 mg kg body weight to about 50 mg/kg body weight. Common dosing frequencies may range, for example, from twice daily to once a week.

In an embodiment, a compound selected from the table 1 ab and Table 1c or composition, comprising such compound, other compound or composition is used in combination with at least one additional agent (for example, an immunotherapy). In an embodiment, the additional agent is administered prior to, overlapping with, concurrently, and/or after administration of a compound selected from the table 1 ab and Table 1c or composition, comprising such compound, other compound or composition. When administered concurrently, a compound selected from the table 1 ab and Table 1c or composition, comprising such compound, other compounds or composition and additional agent are administered in a single formulation or in separate formulations, and if administered separately, then optionally, by different modes of administration. In an embodiment, the combination of a compound selected from the table 1 ab and Table 1c or composition, comprising such compound, other compound or composition of this disclosure and one or more other agents act synergistically.

Further discussion of optimization of dosage and treatment regimens can be found in Benet et al., in Goodman & Gilman's The Pharmacological Basis of Therapeutics, Ninth Edition, Hardman et al., Eds., McGraw-Hill, New York, (1996), Chapter 1, pp. 3-27, and L. A. Bauer, in Pharmacotherapy, A Pathophysiologic Approach, Fourth Edition, DiPiro et al., Eds., Appleton & Lange, Stamford, Conn., (1999), Chapter 3, pp. 21-43, and the references cited therein.

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising,” are to be construed in an open, inclusive sense, that is, as “including, but not limited to.” In addition, the term “comprising” (and related terms such as “comprise” or “comprises” or “having” or “including”) is not intended to exclude that in other certain embodiments, for example, an embodiment of any composition of matter, composition, method, or process, or the like, described herein, may “consist of or “consist essentially of the described features. Headings provided herein are for convenience only and do not interpret the scope or meaning of the claimed embodiments.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Also, as used in this specification and the appended claims, the singular forms “a,” “an,” and “the” include plural referents unless the content clearly dictates otherwise. Thus, for example, reference to “a non-human animal” may refer to one or more non-human animals, or a plurality of such animals, and reference to “a cell” or “the cell” includes reference to one or more cells and equivalents thereof (e.g., plurality of cells) known to those skilled in the art, and so forth. When steps of a method are described or claimed, and the steps are described as occurring in a particular order, the description of a first step occurring (or being performed) “prior to” (i.e., before) a second step has the same meaning if rewritten to state that the second step occurs (or is performed) “subsequent” to the first step. The term “about” when referring to a number or a numerical range means that the number or numerical range referred to is an approximation within experimental variability (or within statistical experimental error), and thus the number or numerical range may vary between 1% and 15% of the stated number or numerical range. For example, the use of “about X” shall encompass +/−1%, 2%, 3%, 4%, 5%, 6%, 7%, 8%, 9%, 10%, 11%, 12%, 13%, 14% and 15% of the value X. It should also be noted that the term “or” is generally employed in its sense including “and/or” unless the content clearly dictates otherwise. The term, “at least one,” for example, when referring to at least one compound or to at least one composition, has the same meaning and understanding as the term, “one or more.

Disclosed herein are compounds, compositions and combinations, kits, and methods for anti-aging treatment and related mediums and methods.

It is also known in the field that the effect of lifespan extension goes together with other anti-aging effects, such as, but not limited to healthspan extension, rejuvenation, prevention and treatment of diverse age-related diseases, disorders and declines (including but not limited to Alzheimer's, Parkinson's, and Huntington's diseases, cardiovascular disease, renal failure, muscle wasting [cachexia], osteopenia or osteoporosis, obesity, insulin resistance or diabetes, and diverse adult-onset cancers).

### Definitions

In some embodiments, the term “protocol” is used in the meaning—the combination of formulation, dosage, regimen, root of administration used. For example, in such embodiment, the reference that the compound is used by the same protocol as it was used in phase 2 clinical trials means that such compound is used in the same formulation, dosage, regimen and root of administration as it was used in such phase 2 clinical trial.

In some embodiments, the term “Targets” means all the following proteins: CDK7, CHEK2, CAPN1, CTSB, HSP90AB1, HSP90AA1, HSPA5, NFKB1, NPC1, PABPC1, ABCB11, ABCB1, PLK1, PLK3, ADRA2A, ADRA2C, ADRA2B, TUBB6, SENP8, SENP6, SENP7, MMP14, MMP13, MMP8, MMP3, MMP1, MAPKAPK2, CTSL, CTSK, CTSS, ATXN2, TBXAS1, CREBBP, PDGFRA, FLT4, FLT1, KDR, PDGFRB, FLT3, KIT, CSF1R, IKBKB, IKBKE, PGGT1B, FAAH, GFER, MAPK14, MAPK12, PRKAA1, ABCG2, ADAM17, BACE2, BACE1, TUBB6, CA7, CA2, CA1, CA5A, CA12, CA9, CA4, CTSB, FEN1, HDAC1, HDAC2, HDAC3, CBX1, SLC6A4, SLC6A2, SLC6A3, PPARD, PPARG, PPARA, PSEN2, HTR7, ADRA1B, SMN2, FYN, SRC, LCK, LYN, TARDBP, CLK2, CLK4, USP2, GBA, RECQL, BLM, WRN, CDK2, PRKAG3, TFPI, LTA4H, DPP7, BACE2, BACE1, CAPN1, CTSB, IGF1R, INSR, EYA2, HSPA5, NFKB1, TCF7L2, TARDBP, BRD4, ABCB1, ADRA1A, ADRA1B, ADRA1D, BRD2, BRD3, BRD4, DRD2, DRD3, DRD4, DRD5, EBP, EGFR, EPHA6, EPHA8, ERBB2, HDAC1, HDAC10, HDAC11, HDAC2, HDAC3, HDAC4, HDAC5, HDAC6, HDAC7, HDAC8, HDAC9, HRH1, HSP90AA1, HTR2A, HTR2B, HTR2C, HTR5B, HTR6, HTR7, KCNH2, MAP4K5, PIK3C2A, PIK3C2B, PIK3CA, PIK3CB, PIK3CD, PIK3CG, PIK3R1, PRKDC, SLC6A2, SLC6A3, SLC6A4. In some embodiments, Target means any one protein selected from the group consisting of Targets.

The term “subject,” as used herein, generally refers to an animal, such as a mammalian species (e.g., mouse or human) or avian (i.e., bird) species, nematode (e.g., C. elegans), or other organism, such as a plant. More specifically, the subject can be a vertebrate, e.g., a mammal such as a mouse, a primate, a simian or a human. Animals include, but are not limited to, farm animals, sport animals, and pets. A subject can be a healthy individual, an individual that has or is suspected of having a disease or a predisposition to the disease, or an individual that is in need of therapy or suspected of needing therapy, or an aged or frail individual. A subject can be any human being.

In some embodiments, by treating or preventing an age-related disease or disorder, any anti-aging treatment is meant to include (but is not limited to) treatments leading to prevention, amelioration or lessening the effects of aging, decreasing or delaying an increase in the biological age, slowing rate of aging; treatment, prevention, amelioration and lessening the effects of frailty or at least one of aging related diseases and conditions or declines or slowing down the progression of such decline (including but not limited to those indicated in Table 1, “Declines”), condition or disease, increasing health span or lifespan, rejuvenation, increasing stress resistance or resilience, increasing rate or other enhancement of recovery after surgery, radiotherapy, disease and/or any other stress, decreasing all-causes or multiple causes of mortality risks or mortality risks related to at least one or at least two of age related diseases or conditions or delaying in increase of such risks, decreasing morbidity risks. The treatment leading to the modulating at least one of biomarkers of aging into more youthful state or slowing down its change into “elder” state is also regarded to be an anti-aging treatment, including but not limited to biomarkers of aging which are visible signs of aging, such as wrinkles, grey hairs etc. In some embodiments, an age-related disease or disorder is selected from: atherosclerosis, cardiovascular disease, adult cancer, arthritis, cataracts, osteoporosis, type 2 diabetes, hypertension, neurodegeneration (including but not limited to Alzheimer's disease, Huntington's disease, and other age-progressive dementias; Parkinson's disease; and amyotrophic lateral sclerosis [ALS]), stroke, atrophic gastritis, osteoarthritis, NASH, camptocormia, chronic obstructive pulmonary disease, coronary artery disease, dopamine dysregulation syndrome, metabolic syndrome, effort incontinence, Hashimoto's thyroiditis, heart failure, late life depression, immunosenescence, myocardial infarction, acute coronary syndrome, sarcopenia, sarcopenic obesity, senile osteoporosis, urinary incontinence etc. Aging-related changes in any parameter or physiological metric are also regarded as age-related conditions, including but not limited to aging related change in blood parameters, heart rate, cognitive functions/decline, bone density, basal metabolic rate, systolic blood pressure, heel bone mineral density (BMD), heel quantitative ultrasound index (QUI), heel broadband ultrasound attenuation, heel broadband ultrasound attenuation, forced expiratory volume in 1-second (FEV1), forced vital capacity (FVC), peak expiratory flow (PEF), duration to first press of snap-button in each round, reaction time, mean time to correctly identify matches, hand grip strength (right and/or left), whole body fat-free mass, leg fat-free mass (right and/or left), and time for recovery after any stress (wound, operation, chemotherapy, disease, change in lifestyle etc.). Aging related change in any parameter of organism is also regarded as an aging related condition, including but not limited to aging related change in at least one of the parameter selected from the Table 1 “Declines”.

In some embodiments, the term “small molecule” means an individual compound with molecular weight less than about 2000 daltons, usually less than about 1500 daltons, more usually less than about 750 daltons, preferably less than about 500 daltons, although molecules larger than 2000 daltons in size will also be included herein.

In some embodiments, the term “senescence associated disease or disorder” is used interchangeably with “aging related disease or condition”. In some embodiments, the term “aging related disease or condition” is used interchangeably with “senescence associated disease or disorder”.

COMBINATION WITH OTHER ANTI-AGING DRUGS: ANY compound of this disclosure can be used with other anti-aging drugs or interventions which will increase anti-aging effects, Some possible geroprotectors include melatonin, carnosine, metformin, nicotinamide mononucleotide (NMN), delta sleep-inducing peptide. NAD+, bASIS™, Rejuvant® LifeTabs®, rapamycin, rapalogs, spermidine and others.

In some embodiments such pharmaceutical composition is for use as an anti-aging medication or for use for the anti-aging treatment or rejuvenation.

In some instances, the pharmaceutical composition described herein is formulated for intravenous administration. Compositions for intravenous administration can comprise a sterile isotonic aqueous buffer. The compositions can also include a solubilizing agent. Compositions for intravenous administration can optionally include a local anesthetic such as lignocaine to lessen pain at the site of the injection. Where the pharmaceutical composition described herein is administered by infusion, it can be dispensed, for example, with an infusion bottle containing sterile pharmaceutical grade water or saline. Where the pharmaceutical composition described herein is administered by injection, an ampule of sterile water for injection or saline can be provided so that the enzyme or enzyme and antioxidant and the carrier can be mixed prior to administration. One of the many possible forms of this invention can be a Lyophilized Concentrate for Intravenous (IV) Injection.

The amount of pharmaceutical composition described herein that is effective for treating a corresponding disease or condition can be determined using standard clinical or pharmacokinetic techniques known to those with skill in the art. In addition, in vitro or in vivo assays can optionally be employed to help identify optimal dosage ranges. The precise dose to be employed can also depend on the route of administration, the disease or condition, the seriousness of the corresponding disease or condition being treated, as well as various physical factors related to the individual being treated, and can be decided according to the judgment of a health-care practitioner. For example, any agent or composition of this disclosure, in an amount ranging from about 0.05 μg/kg to about 100 mg/kg of a patient's body weight, or 0.01 to about 1000 mg/kg of total body weight per day, or from about 0.1 to about 100 mg/kg of total body weight per day, or from about 0.5 to about 15 mg/kg of total body weight per day, or from about 1 mg/kg to about 50 mg/kg of total body weight, which may be administered in one or multiple doses per day or per week or per month or per 6 months or per year or per 3 years or per 8 years or per 12 years or once in a lifetime. In some embodiments, this invention is a pharmaceutical composition and formulation, comprising amount of the agent needed for a subject with weight of 50 kg or in some embodiments 75 kg or in some embodiments 90 kg to provide the amount of the agent described above and at least one pharmaceutically acceptable excipient. Equivalent dosages can be administered over various time periods including, but not limited to, about every 2 hours, about every 4 hours, about every 8 hours, about every 12 hours, about every 24 hours, about every 36 hours, about every 48 hours, about every 72 hours, about every week, about every two weeks, about every three weeks, about every month, and about every two months or every 6 months or every year or every 3 years or every 8 years or every 12 years or once in a lifetime or daily lifelong or as decided by practitioner or patient. The number and frequency of dosages corresponding to a completed course of therapy can be determined according to the judgment of a health-care practitioner. In some embodiments, in case of toxicity or adverse effects the administration can be suspended or dosage decreased until the toxicity or adverse effects disappear and then the administration and/or dosage can be resumed on the previous level.

In some embodiments, the pharmaceutical composition and formulations described herein are administered to a subject by any suitable administration route, including but not limited to, parenteral (e.g., intravenous, subcutaneous, intramuscular), intradermal, intraperitoneal, subcutaneous, intranasal, epidural, sublingual, intravaginal, rectal, by inhalation, topical intracerebral, oral, intranasal, buccal, rectal, or transdermal administration routes. For example, in some instances, the pharmaceutical composition described herein is administered locally. This is achieved, for example, by topical application (including but not limited when prodrugs that require esterase activation after uptake may be applicable topically), local infusion during surgery, by injection, by means of a catheter, by means of a suppository or enema, or by means of an implant, the implant being of a porous, non-porous, or gelatinous material, including membranes, such as sialastic membranes, or fibers. In some situations, the pharmaceutical composition described herein is introduced into the central nervous system, circulatory system or gastrointestinal tract by any suitable route, including intraventricular, intrathecal injection, paraspinal injection, epidural injection, enema, and by injection adjacent to a peripheral nerve. Pulmonary administration can also be employed, e.g., by use of an inhaler or nebulizer, and formulation with an aerosolizing agent, or via perfusion in a fluorocarbon or synthetic pulmonary surfactant.

In some embodiments, the pharmaceutical formulations include, but are not limited to, aqueous fluid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pennsylvania 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins 1999).

In some embodiments, the pharmaceutical formulations further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some embodiments, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the pharmaceutical formulations include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In some embodiments, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, polysorbate-20 or Tween® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil, and polyoxyethylene alkylethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Methods and Uses

Described herein are methods of treating or preventing an age-related disease or disorder or other anti-aging treatment comprising administering to a subject in need thereof an agent or pharmaceutical composition described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

In some embodiments, the compound selected from the table 1 ab and Table 1c and combinations of this disclosure are useful for changing selected biomarkers related to aging or mortality risks into a younger state and thus reducing the risks of mortality, including but not limited to biomarkers described in Table 2, https://www.ncbi.nlm.nih.gov/pmc/articles/PMC5611985/or on the website http://mortalitypredictors.org and the publications cited there on blood predictors of mortality or in any other source.

Every web link cited in this application, in case of inaccessibility can be retrieved via https://web.archive.org or similar internet archive services.

### Kit

In some embodiments this invention is a kit, comprising a compound selected from the table 1 ab and Table 1c or other agent, disclosed or described in this disclosure, or composition disclosed in this application or its functional or structural analog or prodrug and the notice, description or instruction for its use for anti-aging treatment, optionally comprising at least the medication labeling information.

In some embodiments this invention is a kit, comprising a compound selected from the table 1 ab and Table 1c or other agent, disclosed or described in this disclosure, or composition disclosed in this application or its functional or structural analog or prodrug and the notice, description or instruction for its use by human or by other animal subject in a dosage and regimen to maintain concentration of such compound in blood of such subject in 1 μM level at 30% of time, or from 0.5 to 5 μM level at from around 1% of time to around 100% of time, or from 0.5 to 5 μM level at from around 10% of time to around 50% of time, or from 0.05 to 5 μM level or at least one other level described in this disclosure.

The kit of this invention can be made of any material such as paper, plastic, steel, glass, etc, including but not limited to materials used in the art for kits for medications.

In some embodiments, this invention is an compound (agent) of this disclosure with the mentioned notice, description or instruction attached to such agent or imprinted or drawn or in any other way displayed on such agent or in any other way associated with such agent (e.g. in machine readable form). One of the primary purposes of some aspects of his invention is to provide medication for anti-aging treatment.

In some embodiments the contents and appearance of such notice, description or instruction is regulated by the respective national or international rules regarding labeling of medication, incorporated here by reference or such notice, description or instruction comprise at least part or optionally most of the or optionally all the information required by applicable labeling regulations.

The notice, description or instruction, including but not limited to labeling means (e.g., treatment and/or operation guidelines) can be provided in any form that conveys the requisite information. Instruction means can be audio, for example spoken word, recorded in analog or digital form (e.g., audio recording), or received and/or transmitted in analog or digital form (e.g., by telephone, conference call, or audio signal transmitted over a network). Such information can also be visual or video, for example hard-copy (e.g., as a manual, recorded medium, booklet, leaflet, book and the like) or soft-copy (e.g., recorded in analog or digital form as a file recorded on an magnit, electronic, optical, or computer readable medium such as a DVD, disk drive, CD-ROM and the like). Additionally, instruction means can be interactive or real-time (e.g., a teleconference or internet chat or chat bot).

Some kits or agents of this invention can include printed or made in any other way instructions to inform the user of the steps required to properly use it.

In some embodiments, agents and kits, of this invention include a label configured to be coupled to respective agent and kits of this invention. The label includes a first surface and a second surface. In some embodiments, the first surface can be coupled to an outer surface of agents and kits of this invention.

In some embodiments, for example, the first surface can include an adhesive. The second surface can include a textual indicia, such as, for example, a description of the of agents and kits of this invention, a mark indicating its manufacturer or distributor and/or an instruction associated with the use of such of agents and kits of this invention. The label can further include an electronic circuit system configured to output an electronic signal. In some embodiments, the electronic signal can include an instruction associated with the use of the mediums, kits, devices or agents of this invention.

In some embodiments the instruction is an instruction for use as anti-aging medication. In some embodiments the instruction is an instruction for use as medication for a treating or preventing an age-related disease or disorder.

In some embodiments, the notice, description or instruction, including but not limited to labeling can be shown on the lenses, computer glasses, transmitted via brain computer interface or by any other means or can be encoded by the Quick Response Code or any other machine readable form.

The notice, description or instruction, including but not limited to labeling, can be implemented in digital electronic circuitry, or in computer firmware, hardware, software, or in combinations thereof. The implementation can be as a computer program product, e.g., a computer program tangibly embodied in an information carrier, e.g., in a machine-readable storage device or in a propagated signal, for execution by, or to control the operation of, data processing apparatus, e.g., a programmable processor, a computer, or multiple computers. A computer program can be recorded in any form of programming language, including compiled or interpreted languages, and the computer program can be deployed in any form, including as a stand-alone program or as a subroutine, element, or other unit suitable for use in a computing environment. A computer program can be deployed to be executed on one computer or on multiple computers at one site or several sites.

The notice, description or instruction, including but not limited to labeling can be performed by one or more programmable processors executing a computer program to perform functions of the invention by operating on input data and generating output. It can also be performed by, and an apparatus can be implemented as, special purpose logic circuitry, e.g., an FPGA (field programmable gate array) or an ASIC (application-specific integrated circuit). Subroutines can refer to portions of the computer program and/or the processor/special circuitry that implements that functionality.

The dosage levels and mode of administration will be dependent on a variety of factors such as the treatment used, the device, the active agent, the context of use (e.g., the patient to be treated), and the like. Optimization of modes of administration, dosage levels, and adjustment of protocols, including monitoring systems to assess effectiveness are routine matters well within ordinary skill.

Further discussion of optimization of dosage and treatment regimens can be found in Benet et al., in Goodman & Gilman's The Pharmacological Basis of Therapeutics, Ninth Edition, Hardman et al., Eds., McGraw-Hill, New York, (1996), Chapter 1, pp. 3-27, and L. A. Bauer, in Pharmacotherapy, A Pathophysiologic Approach, Fourth Edition, DiPiro et al., Eds., Appleton & Lange, Stamford, Conn., (1999), Chapter 3, pp. 21-43, and the references cited therein, to which the reader is referred.

The compound s of this invention are known in the art as well as the process of its manufacturing. The compound s of this invention can also be acquired from commercial sources.

In some embodiments, the one or two or more biomarkers (optionally—with associated measurement units in plasma) which will be changed into more youthful state as a result of administration of compound or combination of this invention is a biological age characteristic. In some embodiments, biological age characterizes the health status of the subject.

In some embodiments, a compound selected from the table 1 ab and Table 1c or the agents and combinations of this disclosure are administered by aged subject.

In some embodiments, the biological age determination approach is described in prior art, including but not limited to any of the following publications https://www.ncbi.nlm.nih.gov/pmc/articles/PMC5514388/, https://www.ncbi.nlm.nih.gov/pmc/articles/PMC4931851/, https://www.ncbi.nlm.nih.gov/pmc/articles/PMC5514388/and corresponding references related to blood based biological age determination.

In some embodiments, the biological age is understood as the distance measured along a continuous trajectory consisting of distinct phases, each corresponding to subsequent human life stages as described in more details in https://www.biorxiv.org/content/biorxiv/early/2017/09/09/186569.full.pdf

In some embodiments, this at least one of the compound, composition or combination of this disclosure reduces mortality risk or high mortality risk in about 1 month, in about 3 months, in about 6 months, in about 1 year, from about 1 month to about 6 months, from about 1 month to about 1 year, from about 1 year to about 3 years, from about 3 years to about 5 years, from about 5 years to about 8 years, from about 5 years to about 10 years, in about 5 years, in about 10 years, in about 15 years. In some embodiments, mortality risk is a risk of dying from age related condition or disease. In some embodiments, mortality risk is all cause mortality risk. Non limiting examples of biomarkers of mortality and its critical volumes are described in prior art, including but not limited to https://www.ncbi.nlm.nih.gov/pmc/articles/PMC4899173/, https://www.ncbi.nlm.nih.gov/pmc/articles/PMC4454670/, https://www.ncbi.nlm.nih.gov/pmc/articles/PMC5334528/
Maximus Peto, Carlos De la Guardia, Ksenia Winslow, Andrew Ho, Kristen Fortney, & Eric Morgen. “Mortalitypredictors.org, a manually curated database of published biomarkers of human all-cause mortality. Aging, 2017.

In some embodiments, a compound selected from the table 1 ab and Table 1c composition or combination of this disclosure reduces morbidity risk in about 1 month, in about 3 months, in about 6 months, in about 1 year, from about 1 month to about 6 months, from about 1 month to about 1 year, from about 1 year to about 3 years, from about 3 years to about 5 years, from about 5 years to about 8 years, from about 5 years to about 10 years, in about 5 years, in about 10 years, in about 15 years. In some embodiments, morbidity risk is a risk of acquiring an age related condition or disease.

In some embodiments this invention is a method, comprising administration of the compound selected from the table 1 ab and Table 1c, composition or combination of this disclosure, for the at least one of the selected from the group consisting of: reducing biological age of the patient, improving of at least one aging biomarker, alleviating of at least one age related deficit or disease, improving of at least one of rejuvenation marker, treating frailty, increasing health span or life span, providing an anti-aging treatment.

In some embodiments, this invention is at least one of the of the compounds selected from the table 1 ab and Table 1c, composition or combination of this disclosure useful for treatment and prevention of disease selected from the group: type 2 diabetes, age-related cardiovascular diseases, including but not limited to ischemic heart disease and stroke, metabolic syndrome, COPD, Alzheimer's disease etc., including but not limited those mentioned in this disclosure or at least one of the aging related declines. In some embodiments, treatment by the compound selected from the table 1 ab and Table 1c may include treatment of any disease or condition which is fully or partially mediated by the induction or maintenance of a non-proliferating or senescent state in a cell or a population of cells in a subject. Non-limiting examples include age-related tissue or organ decline which may lack visible indication of pathology, or overt pathology such as a degenerative disease or a function-decreasing disorder. For example, Alzheimer's disease, Parkinson's disease, cataracts, macular degeneration, glaucoma, atherosclerosis, acute coronary syndrome, myocardial infarction, stroke, hypertension, idiopathic pulmonary fibrosis (IPF), chronic obstructive pulmonary disease (COPD), osteoarthritis, type 2 diabetes, obesity, fat dysfunction, coronary artery disease, cerebrovascular disease, periodontal disease, and cancer treatment-related disability such as atrophy and fibrosis in various tissues, brain and heart injury, and therapy-related myelodysplastic syndromes. Additionally, an age-related pathology may include an accelerated aging disease such as Hutchinson-Gilford progeria syndrome, Werner syndrome, Cockayne syndrome, exroderma pigmentosum, ataxia telangiectasia, Fanconi anemia, dyskeratosis congenital, aplastic anemia, idiopathic pulmonary fibrosis, and others. A method of identifying an age-related disease or condition as described herein may include detecting the presence of senescent cells.

In some embodiments, a senescence-associated pathology that can be treated by compound selected from the table 1 ab and Table 1c may include any disease or condition which is fully or partially mediated by the induction or maintenance of a non-proliferating or senescent state in a cell or a population of cells in a subject. Non-limiting examples include cardiovascular diseases such as angina, aortic aneurysm, arrhythmia, brain aneurysm, cardiac diastolic dysfunction, cardiac fibrosis, cardiac stress resistance, cardiomyopathy, carotid artery disease, coronary thrombosis, endocarditis, hypercholesterolemia, hyperlipidemia, mitral valve prolapsed, and peripheral vascular disease; inflammatory or autoimmune diseases such as herniated intervertebral disc, inflammatory bowel disease, kyphosis, oral mucositis, lupus, interstital cystitis, scleroderma, and alopecia; neurodegenerative diseases such as dementia, Huntington's disease, motor neuron dysfunction, age-related memory decline, and depression/mood disorders; metabolic diseases such as diabetic ulcer and metabolic syndrome; pulmonary diseases such as age-related loss of pulmonary function, asthma, bronchiectasis, cystic fibrosis, emphysema, and age-associated sleep apnea; gastrointestinal diseases such as Barrett's esophagus; age-related disorders such as liver fibrosis, muscle fatigue, oral submucosa fibrosis, pancreatic fibrosis, benign prostatic hyperplasia (BPH), and age-related sleep disorders; reproductive disorders such as menopause (male and female), egg supply (female), sperm viability (male), fertility (male and female), sex drive, and erectile function and arousal (male and female); dermatological diseases such as atopic dermatitis, cutaneous lupus, cutaneous lymphomas, dysesthesia, eczema, eczematous eruptions, eosinophilic dermatosis, fibrohistocytic proliferations of skin, hyperpigmentation, immunobullous dermatosis, nevi, pemphigoid, pemphigus, pruritis, psoriasis, rashes, reactive neutrophiliedermatosis, rhytides, and urticarial; and other diseases such as diabetic wound healing, post-transplant kidney fibrosis, and carotid thrombosis.

In some embodiments, a method is provided for treating a senescence associated disease or disorder in a subject comprising administering to the subject a compound selected from the table 1 ab and Table 1c or combination, combination, comprising such compound; wherein the senescence associated disease or disorder is not a cancer, wherein such compound or combination is administered during a treatment course of 1-7 days every 0.5-12 months; provided that if the senescence associated disease or disorder is a senescence associated metabolic disorder, such compound or combination is administered during a treatment course of 1-7 days every 4-12 months. In certain embodiments, such compound or combination is administered once every 0.5-12 months; provided that if the senescence associated disease or disorder is a senescence associated metabolic disorder, such compound or combination is administered once every 4-12 months. In other certain embodiments, the aging related disease or senescent cell-associated disease or disorder is a cardiovascular disease or disorder, inflammatory disease or disorder, a pulmonary disease or disorder, a neurological disease or disorder. In a specific embodiment, the cardiovascular disease or disorder is atherosclerosis. In another specific embodiment, the inflammatory disease or disorder is osteoarthritis. In another specific embodiment, the pulmonary disease or disorder is idiopathic pulmonary fibrosis or chronic obstructive pulmonary disease. In another specific embodiment, the neurological disease or disorder is selected from mild cognitive impairment; motor neuron dysfunction; Alzheimer's disease; Parkinson's disease; and macular degeneration. In another specific embodiment, the senescence associated metabolic disease or disorder is selected from diabetes, metabolic syndrome, and obesity. In another specific embodiment, the aging related disease or senescence-associated disease or disorder is a dermatological disease or disorder is selected from eczema, psoriasis, hyperpigmentation, nevi, rashes, atopic dermatitis, urticaria, diseases and disorders related to photosensitivity or photoaging, rhytides; pruritis; dysesthesia; eczematous eruptions; eosinophilic dermatosis; reactive neutrophilic dermatosis; pemphigus; pemphigoid; immunobullous dermatosis; fibrohistocytic proliferations of skin; cutaneous lymphomas; and cutaneous lupus.

In some embodiments, a method is provided for treating a senescence-associated metabolic disease or disorder in a subject comprising administering to the subject a compound selected from the table 1 ab and Table 1c or combination, comprising such compound, wherein such compound or combination is administered during a treatment course of 1-7 days every 4-12 months, and wherein the metabolic disease or disorder is selected from diabetes, metabolic syndrome, and obesity. In a specific embodiment, such compound or combination is administered once every 4.12 months. In another specific embodiment, the senescent cell is selected from a senescent fibroblast, a senescent pre-adipocyte, a senescent epithelial cell, a senescent chondrocyte, a senescent neuron, and a senescent endothelial cell. In another specific embodiment, the senescent cell is a senescent pre-adipocyte.

In some embodiments, a method is provided for treating, reducing the likelihood of occurrence of, or delaying onset of age related disease or decline or a senescent cell-associated disease or disorder in a subject who has an age related disease or decline a senescent cell-associated disease or disorder or who has at least one predisposing factor for developing the senescent cell-associated disease or disorder, comprising administering to the subject a compound selected from the table 1 ab and Table 1c or combination, comprising such compound, thereby promoting death of the senescent cell, wherein the senescent cell-associated disease or disorder is a cardiovascular disease or disorder, inflammatory disease or disorder, a pulmonary disease or disorder, a neurological disease or disorder, with the proviso that if the subject has a cancer, and such compound is not a primary therapy for treating the cancer, and wherein the such compound or composition is administered once every 0.5-12 months.

In some embodiments, a method is provided for treating, reducing the likelihood of occurrence of, or delaying onset of a senescent cell-associated disease or disorder in a subject who has a senescent cell-associated disease or disorder or who has at least one predisposing factor for developing the senescent cell-associated disease or disorder, comprising administering to the subject a senolytic combination comprising (a) a first agent that alters either one or both of a cell survival signaling pathway and an

In some embodiments, a method is provided for killing a senescent cell comprising contacting the senescent cell and a compound selected from the table 1 ab and Table 1c or combination, comprising such compound, thereby promoting death of the senescent cell, wherein the senescence cell is present in a subject who has a senescent cell-associated disease or disorder or who has at least one predisposing factor for developing the senescent cell-associated disease or disorder, with the proviso that if the subject has a cancer, and such compound or is not a primary therapy for treating the cancer, wherein the senescent cell-associated disease or disorder is a cardiovascular disease or disorder, inflammatory disease or disorder, a pulmonary disease or disorder, a neurological disease or disorder, wherein the combination is administered once every 0.5-12 months.

In certain embodiments of the methods described above and herein, the senescent cell-associated disease or disorder is selected from atherosclerosis; osteoarthritis; idiopathic pulmonary fibrosis; chronic obstructive pulmonary disease; mild cognitive impairment; motor neuron dysfunction; Alzheimer's disease; Parkinson's disease; and macular degeneration.

In another embodiment, a method is provided for killing a senescent cell comprising contacting the senescent cell and compound selected from the table 1 or combination, comprising such compound, thereby promoting death of the senescent cell, wherein the senescence cell is present in a subject who has a senescent cell-associated disease or disorder or who has at least one predisposing factor for developing the senescent cell-associated disease or disorder, wherein the senescent cell-associated disease or disorder is a metabolic disorder selected from diabetes, metabolic syndrome, and obesity, and wherein the combination is administered once every 4-12 months.

In another embodiment, a method is provided for treating or reducing the likelihood of occurrence of atherosclerosis in a subject who has atherosclerosis or who has at least one predisposing factor for developing atherosclerosis, comprising administering to the subject a compound selected from the table 1 ab and Table 1c or combination, comprising such compound, thereby promoting death of the senescent cell, wherein the combination is administered once every 0.5.12 months, and wherein the first and second agents are different.

In certain embodiments of the methods described above and herein, the senescent cell is selected from a senescent fibroblast, a senescent pre-adipocyte, a senescent epithelial cell, a senescent chondrocyte, a senescent neuron, a senescent smooth muscle cell, a senescent mesenchymal cell, a senescent macrophage, and a senescent endothelial cell. In certain specific embodiments, the senescent cell is a senescent pre-adipocyte.

In some embodiments this invention is a method, including but not limited to method of testing of efficacy of compound, composition or combination of this disclosure, comprising the checking in the subject treated by such therapy at least one of the following: checking biological age of the patient, at least one aging biomarker, at least one age related deficit or disease, at least one of rejuvenation marker, frailty, health span or life span, or any other marker or parameter reasonable for checking in testing of anti-aging therapy efficacy.

In some embodiments, this invention is a method of anti-aging treatment, comprising administering by subject at least one compound, composition or combination of this disclosure, and repeating administration in case biological age increased for more than 1 year, more than 3 years, more than 8 years, more than 10 years. In some embodiments the dosage and regimen for implementing disclosed method is defined to keep biological age at the reasonably minimum level, or close to the age that subject had at the time of first treatment.

In some embodiments checking of efficacy of compound, composition or combination of this disclosure and measurement of markers or symptoms of related diseases or conditions is conducted in 1 month after the infusion of treated plasma or administration of therapy in therapeutically effective amount, in 3 months, in 6 months, in 12 months, in 18 months, in 24 months or in 36 months after such infusion, or in around such date, or in date reasonably defined by the practitioner based on the parameter being measured and other factors known to the expert in the field.

Any of the compound s and combinations of this disclosure can be provided in formulations defined by the expert skilled in the art, including but not limited those known in the art and those suggested in the examples.

In some embodiments, compounds selected from the table 1 ab and Table 1c or their structural or functional analogs or prodrugs and other agents of this disclosure to be useful for anti-aging or treatment of age related disease or other disease indicated in this disclosure shall be administered in the same dosages as such agents are effective in their primary indication (Primary indication effective dosage or PIED), which is known in the art. In some embodiments, the dosage for the agent of this disclosure should be at least 100 times less or least 50 times less or at least 10 times less or at least 5 times less, or at least 2 times less, or at least 50% less, or at least 25% less, or at least 10% less, or at least 5 times more, or at least 2 times more, or at least 50% more, or at least 25% more, or at least 10% more, or at least 10 times more, or at least 100 times more than PIED to have anti-aging therapeutic effect. In some embodiments, to have anti-aging therapeutic effect such agent shall be administered at maximum tolerated dose.

| patient ID | Name | Date of birth | diagnosis | Treatment prescribed |
|---|---|---|---|---|
| 28282839 | John | 2 Jan. 1937 | Before treatment: | The compound selected from the |
| | Junior | | moderate frailty, | group consisting of all compounds |
| | Smith | | moderate cognitive | listed in table 1 ab and Table 1c |
| | | | decline, hand grip | |
| | | | strength - 21.3 kg | |
| | | | and other age related | |
| | | | deficites | |
| 28282838 | peter | 2 Jan. 1937 | Before treatment: | The compound selected from the |
| | Junior | | covid-19, influenza, | group consisting of all compounds |
| | Smith | | pulmonary fibrosis | listed in table 1 ab and Table 1c |
| 28282837 | Val | 2 Jan. 1937 | Before treatment: | INDORAMIN |
| | Smith | | moderate frailty, | |
| | | | moderate cognitive | |
| | | | decline, hand grip | |
| | | | strength - 21.3 kg | |
| | | | and other age related | |
| | | | deficites | |
| 28282836 | Robert | 2 Jan. 1937 | Before treatment: | PHENTOLAMINE |
| | Smith | | covid-19, influenza, | |
| | | | pulmonary fibrosis | |
| 28282835 | John | 2 Jan. 1937 | Before treatment: | DEXMEDETOMIDINE |
| | Junior | | moderate frailty, | |
| | Smith | | moderate cognitive | |
| | | | decline, hand grip | |
| | | | strength - 21.3 kg | |
| | | | and other age related | |
| | | | deficites | |
| 28282834 | Donald | 2 Jan. 1937 | Before treatment: | YOHIMBINE |
| | Junior | | covid-19, influenza, | |
| | Smith | | pulmonary fibrosis | |

In some embodiments, PIED is a dosage which was tested in Phase 1. In some embodiments, PIED is a dosage which proved to be safe in Phase 1.

In some embodiments, the dosage for the compound selected from the table 1 ab and Table 1c should be selected from the group consisting of: about at least 1000 times less at least 100 times less or least 50 times less or at least 10 times less or at least 5 times less, or at least 2 times less, or at least 50% less, or at least 25% less, or at least 10% less, than maximum tolerated dose to have anti-aging therapeutic effect or to be effective against aging, frailty, aging related disease or condition or other disease or condition mentioned in this application.

In some embodiments this invention is a tangible medium comprising a computer program, which, when executed, causes a medium to perform a method comprising: attribution to the information about a subject an information about an anti-aging treatment related to compound, composition or combination of this disclosure, optionally further comprising attributing to the information about patient before or after or before and after the treatment to information about checking of at least one selected from the group: biological age of the patient, at least one aging biomarker, at least one age related deficit or disease or its symptom, at least one of rejuvenation marker, frailty, health span or life span.

A prophetic example of such tangible medium could be a APPLE™ 2014 MACBOOK AIR™ 13″ Intel™ i5 with Microsoft™ Excel™ installed and executed on it, wherein to patient with name John Junior Smith (born 2 Jan. 1937) the information about using compound selected from the table 1 ab and Table 1c or their pharmaceutically acceptable salt or any combination of it or compounds or, optionally, having the similar SAR characteristics in therapeutically effective amount is attributed in the sense that is logically linked as an information in Excel table (in this example attribution is realized as placing the information about treatment by such drug in the same line in the file with the name and ID of the patient to whom such treatment is prescribed) and allows easy finding of patients to whom such treatment is prescribed and other processing of such information.

Processors suitable for the execution of a computer program related to this invention include, by way of example, both general and special purpose microprocessors, and any one or more processors of any kind of digital computer. Generally, a processor receives instructions and data from a read-only memory or a random access memory or both. The essential elements of a computer are a processor for executing instructions and one or more memory devices for storing instructions and data. Generally, a computer also includes, or be operatively coupled to receive data from or transfer data to, or both, one or more mass storage devices for storing data, e.g., magnetic, magneto-optical disks, or optical disks. Data transmission and instructions can also occur over a communications network. Information carriers suitable for embodying computer program instructions and data include all forms of non-volatile memory, including by way of example semiconductor memory devices, e.g., EPROM, EEPROM, and flash memory devices;
magnetic disks, e.g., internal hard disks or removable disks; magneto-optical disks; and CD-ROM and DVD-ROM disks. The processor and the memory can be supplemented by, or incorporated in special purpose logic circuitry.

In some embodiments this invention is a tangible medium comprising a computer program, which, when executed, causes a device to perform a method comprising: attribution to the information regarding compound, composition or combination of this disclosure, an information about anti-aging treatment.

As an example of such attribution the excel file on the same computer as described above or Internet site hosted on server that displays the following can be suggested

| | Agent/Mechanism of | |
|---|---|---|
| Name | Action/Mode of action | Indication |
| ASD1 | Compound selected | anti-aging, anti-frailty, amelioration of moderate |
| | from the table 1 ab | cognitive decline, amelioration hand grip strength |
| | and Table 1c | lose and amelioration of other age related deficits |
| GER23 | OLANZAPINE | anti-aging, anti-frailty, amelioration of moderate |
| | | cognitive decline, amelioration hand grip strength |
| | | lose and amelioration of other age related deficits |
| GER56 | PERPHENAZINE | anti-aging, anti-frailty, amelioration of moderate |
| | | cognitive decline, amelioration hand grip strength |
| | | lose and amelioration of other age related deficits |
| FDS89 | IFENPRODIL | anti-aging, anti-frailty, amelioration of moderate |
| | | cognitive decline, amelioration hand grip strength |
| | | lose and amelioration of other age related deficits |

In some embodiments this invention is a method, method comprising: attribution to the information about a subject an information about an anti-aging treatment related to compound selected from the table 1 ab and Table 1c, composition or combination of this disclosure, optionally further comprising attributing to the information about patient before or after or before and after the treatment to information about checking of at least one selected from the group: biological age of the patient, at least one aging biomarker, at least one age related deficit or disease, at least one of rejuvenation marker, frailty, health span or life span. In some embodiments such method is a computer implemented method.

In some embodiments this invention is a method, the method of this invention, comprising attribution of information executed on the medium of this invention and described in corresponding part of this disclosure related to such medium.

In some embodiments this invention is a tangible medium or computer system or processor, comprising a computer program, which, when executed, causes a medium to perform a method comprising attribution of information described in this disclosure.

In some embodiments this invention is an apparatus to execute method described in this disclosure, the apparatus comprising the processor comprising the tangible medium described in this disclosure

In some embodiments, dose for anti-aging effect of the compound selected from the table 1 ab and Table 1c is about 500 mg (one tablet) 3-4 times a day. In some embodiment, dose of the compound selected from the table 1 ab and Table 1c is from about 50 mg to about 4000 mg. In some embodiment, dose of the compound selected from the table 1 ab and Table 1c is from about 10 mg to about 4000 mg. In some embodiment, dose of the compound selected from the table 1 ab and Table 1c is from 100 mg to 2000 mg. In some embodiment, dose of the compound selected from the table 1 ab and Table 1c is from about 1 mg to about 100 mg. In some embodiment, dose of the compound selected from the table 1 ab and Table 1c is from about 10 mg to about 50 mg. In some embodiments, this invention is a pharmaceutical composition or formulation comprising single dosage in the amount described above and at least one pharmaceutically acceptable excipient.

In some embodiments, for anti-aging effect the compound selected from the table 1 ab and Table 1c is administered intramuscularly. In some embodiments, the single dose for all age groups is 8-16 mg/kg body weight according to the following scheme: 16-31 kg body weight-250 mg the compound selected from the table 1 ab and Table 1c (½ ml); 32-46 kg body weight-500 mg the compound selected from the table 1 ab and Table 1c (1 ml); 47-62 kg body weight—from about 500 to about 750 mg the compound selected from the table 1 ab and Table 1c (1-1.5 ml); and over 63 kg body weight-750-1000 mg the compound selected from the table 1 ab and Table 1c (1.5-2 ml). In some embodiments, the dose can be repeated in 6-8 hours. In some embodiments, the dose in adults is 250-500 mg (½-1 tablet) 2 or 3 times daily. In some embodiments, maximal 24-hour dose is about amount selected from the group consisting of: from about 0.01 mg to about 0.1 mg, from about 0.1 mg to about 1 mg, from about 1 mg to about 10 mg, from about 10 mg to about 100, from about 100 mg to about 1000 mg, from about 1 g to about 10 g, from about 0.01 mg to about 10 g. In some embodiments the compound selected from the group consisting of all compounds listed in table 1 ab and Table 1c is administered from 5 mg/m2 to 200 mg/m2. In some embodiments, the compound selected from the group consisting of all compounds listed in table 1 ab and Table 1c is administered at a dose shown above or of therapeutic efficacy, 2-3 times daily.

In some embodiments, the compound selected from the group consisting of all compounds listed in table 1 ab and Table 1c can be administered as a shot into the fatty part of the skin. In some embodiments, The compound selected from the group consisting of all compounds listed in table 1 ab and Table 1c can be administered as an infusion into a vein over a period of time.

In some embodiments the compound selected from the group consisting of all compounds listed in table 1 ab and Table 1c can be administered in the dosage from about 0.1 mg/m2/day to about 1000 mg/m2/day or from about 1 mg/m2/day to about 100 mg/m2/day via subcutaneous injection or IV infusion for about from 1 day to about 365 days.

In some embodiments for achievement of anti-aging effect the administration of the compound selected from the group consisting of all compounds listed in table 1 ab and Table 1c should be repeated cycles about every 4 weeks, or about every 6 weeks, about every 8 weeks or about every 16 weeks, about every 1 year, about every 2 years, about every 5 years, about every 8 years.

In some embodiments, the compound selected from the group consisting of all compounds listed in table 1 ab and Table 1c or its pharmaceutically acceptable salt or any combination of it or compounds or, optionally, having the similar SAR characteristics or any of its combination to achieve anti-aging effect can be administered by human or by other animal subject in a dosage and regimen to create or in some other embodiment to maintain a concentration of such compound in blood of such subject in 0.01 μM level, or in 0.03 μM level, or in 0.1 μM level, or in 0.02 μM level, or in 0.04 μM level, or in 0.08 UM level, or in 0.1 μM level, or in 0.2 μM level, or in 0.4 μM level, or in 0.8 μM, level, or in 1 μM level, or in 2 μM level, or in 4 μM level, or in 8 μM level, or in 10 μM level, or in 12 μM level, or in 14 μM level, or in 18 μM level, or in 20 μM level, or from 0.01 μM to 0.05 μM level, or from 0.01 μM to 0.1 μM level, or from 0.01 μM to 1 μM level, or from 0.1 μM to 1 μM level, or from 0.5 μM to 5 μM level, or from about 0.5 μM to about 50 μM level, or in about level of +/−10% of the indicated above; in some embodiments such concentration should be maintained in blood for about 1 minute, or for about 10 minutes, or for about 1 hour, or for about 2 hours, or for about 4 hours, or for about 8 hours, or for about 16 hours, or for about 24 hours, or for about 48 hours, or for about 1 week, or for about 2 weeks, or for about 4 weeks, or for about 8 weeks, or for about 16 weeks, or for about 1 year, or for about 2 years, or for about 5 years, or lifelong; in some embodiments such concentration should be maintained in blood for about 10% of time, or for about 20% of time, or for about 30% of time, or for about 40% of time, or for about 50% of time, or for about 60% of time, or for about 70% of time, or for about 80% of time, or for about 90% of time, or for about 100% of time, or for about from 1% of time to 10% of time, or for about from 1% of time to about 90% of time, or for about from 10% of time to 50% of time, wherein time is selected from about 1 second, about 1 minute, or about 10 minutes, or about 1 hour, or about 2 hours, or about 4 hours, or about 8 hours, or about 16 hours, or about 24 hours, or about 48 hours, or about 1 week, or about 2 weeks, or about 4 weeks, or about 8 weeks, or about 16 weeks, or about 1 year, or about 2 years, or about 5 years, or from about 1 second to about 1 day, or from about 1 second to about 1 minute, or from about 1 minute to about 1 hour, or from about 1 hour to about 24 hours, or from about 24 hours to about 1 week, or from about 1 week to about 1 month, or from about 1 month to about 1 year, or from about 1 year to about 5 years, or lifelong.

In some embodiments, this invention is a delivery device or dosing device delivering the compound selected from the group consisting of all compounds listed in table 1 ab and Table 1c or their pharmaceutically acceptable salt or any combination of it or compounds or, optionally, having the similar SAR characteristics or any of its combination, and enabling maintaining a concentration of such compound in blood of such subject in 1 μM level at 30% of time, or from 0.5 to 5 μM level at from around 1% of time to around 100% of time, or from 0.5 to 5 μM level at from around 10% of time to around 50% of time or concentration described in this disclosure for a time described in this disclosure. A non-limiting example of such device—analog of insulin pump, automatically or semi-automatically or manually injecting the compound selected from the group consisting of all compounds listed in table 1 ab and Table 1c or their pharmaceutically acceptable salt or any combination of it or compounds or, optionally, having the similar SAR characteristics or any of its combination to maintain the a concentration of such compound in blood of such subject in 1 μM level at 30% of time, or from 0.5 to 5 μM level at from around 1% of time to around 100% of time, or from 0.5 to 5 μM level at from around 10% of time to around 50% of time or a concentration described in this disclosure for a time described in this disclosure.

In some embodiments, this invention is implantable medical device for controlled delivery of therapeutic agents of this disclosure. One of many examples of this invention can have a titanium reservoir, and a porous titanium oxide based membrane to control the rate of release of the therapeutic agent of this invention. The reservoir can contain a formulation of the active agent, including a stabilizer for the active agent, wherein the stabilizer is provided in an extended release configuration. Further details on some of the examples of devices for delivery of compounds of this invention to maintain the a concentration of such compound in blood of such subject in 1 μM level at 30% of time, or from about 0.01 to about 500 μM level at from around 1% of time to around 100% of time, or from 0.5 to 5 μM level at from around 10% of time to around 50% of time or a concentration described in this disclosure for a time described in this disclosure can be found e.g. in US application US20160220496A1.

In some embodiments, this invention is a formulation comprising the compound selected from the group consisting of all compounds listed in table 1 ab and Table 1c or their pharmaceutically acceptable salt or any combination of it or compounds or, optionally, having the similar SAR characteristics or any of its combination, enabling maintaining a concentration of such compound in blood of subject in 0.5 μM 1 μM level at 30% of time, or from about 0.05 μM to about 500 μM level at from around 1% of time to around 100% of time, or from 0.5 μM to 5 μM level at from around 10% of time to around 50% of time or a concentration described in this disclosure for a time described in this disclosure.

In some embodiments, this invention is a slow release formulation or prodrug, comprising the compound selected from the group consisting of all compounds listed in table 1 ab and Table 1c or their pharmaceutically acceptable salt or any combination of it or compounds or, optionally, having the similar SAR characteristics or any of its combination, enabling maintaining a concentration of such compound in blood of subject in 1 μM level at 30% of time, or from 0.5 to 5 μM level at from around 1% of time to around 100% of time, or from about 0.05 to about 500 μM level at from around 10% of time to around 50% of time or a concentration described in this disclosure for a time described in this disclosure. In some embodiments, such slow release formulation or prodrug is for anti-aging use.

In some embodiments, this invention is a pharmaceutical composition for oral administration, including but not limited to tablet, capsule, suspension, drink etc., comprising the compound selected from the group consisting of all compounds listed in table 1 ab and Table 1c—about 100 mg, or about 50 mg, or about 75 mg, or from about 10 mg to about 150 mg, or from about 20 mg to about 100 mg, or from about 1 mg to about 150 mg, or from about 0.01 mg to about 1000 mg. In some embodiments, this invention is a pharmaceutical composition, comprising the compound selected from the group consisting of all compounds listed in table 1 ab and Table 1c—about 100 mg, or about 50 mg, or about 75 mg, or from about 10 mg to about 150 mg, or from about 20 mg to 100 mg, or from 1 mg to 150 mg, in some embodiments, this invention such pharmaceutical composition is for anti-aging use.

In some embodiments, the compound selected from the group consisting of all compounds listed in table 1 ab and Table 1c should be administered once daily until disease progression stopped or at least one symptom of age related condition is alleviated or no longer tolerated by the patient.

In some embodiments, this invention is a pharmaceutical composition for oral administration, including but not limited to tablet, capsule, suspension, drink etc., comprising the compound selected from the group consisting of all compounds listed in table 1 ab and Table 1c or their pharmaceutically acceptable salt or any combination of it or compounds or, optionally, having the similar SAR characteristics, in some embodiments, this invention such pharmaceutical composition is for anti-aging use.

In some embodiments, this invention is a pharmaceutical composition and formulation, comprising amount of the compound selected from the group consisting of all compounds listed in table 1 ab and Table 1c or their pharmaceutically acceptable salt or any combination of it or compounds or, optionally, having the similar SAR characteristics in the amount needed for a single dose to provide a concentration described in this disclosure for a time described in this disclosure calculated for a subject with weight of 50 kg or in some embodiments 75 kg or in some embodiments 90 kg and at least one pharmaceutically acceptable excipient.

Any of the compound selected from the table 1 and combinations of this disclosure can be provided in formulations defined by the expert skilled in the art, including but not limited those known in the art and those suggested in the examples.

In some embodiments, this invention the new use of the compound selected from the group consisting of all compounds listed in table 1 ab and Table 1c wherein a new use is a treatment of viral disease, decreasing a mortality of elderly and/or frail patients, reduction of a mortality of elderly and/or frail patients from viral disease, decreasing a mortality of elderly and/or frail patients from covid-19, decreasing a mortality of elderly and/or frail patients from influenza, decreasing a mortality of elderly and/or frail patients from disease, caused by SARS-CoV, SARS-CoV-2 (Severe acute respiratory syndrome coronavirus 2) or other coronavirus, reduction of pulmonary fibrosis caused by viral infection of SARS-CoV, reduction of pulmonary fibrosis caused by viral infection of SARS-CoV-2, reduction of virus titer of SARS-CoV, reduction of virus titer of SARS-CoV-2

In some embodiments, this invention the new use of the compound selected from the group consisting of all compounds listed in table 1 ab and Table 1c, wherein a new use is a alleviation of symptoms of viral disease in elderly and/or frail patients, alleviation of symptoms of viral disease in elderly and/or frail patients, caused by covid-19, alleviation of symptoms of viral disease in elderly and/or frail patients, caused by influenza, alleviation of symptoms of viral disease in elderly and/or frail patients, of disease or condition, caused by SARS-CoV, SARS-CoV-2 (Severe acute respiratory syndrome coronavirus 2) or other coronavirus or other virus or bacteria.

In some embodiments, the compound selected from the group consisting of all compounds listed in table 1 ab and Table 1c is useful for treatment and prevention of disease or condition selected from the group: accelerated aging associated with cancer treatment, cancer survival, cognitive impairment associated with cancer treatment and survival, trauma, stroke, HIV, Down syndrome, schizophrenia, condition associated with the cancer treatment, other diseases and conditions associated with increased frailty.

In some embodiments, the compound selected from the group consisting of all compounds listed in table 1 ab and Table 1c is useful as vaccine adjuvant. Examples are vaccine against influenza A, SARS, SARS cov-2, Tetanus and Diphtheria (Td) vaccine; Tetanus, Diphtheria, and Pertussis (Tdap) vaccine; Shingles vaccine; Pneumococcal polysaccharide vaccine; Influenza (flu) vaccine. and vaccines from other infections. In some embodiments, such vaccine adjuvant improves immune response to vaccine.

In some embodiments, the compound selected from the group consisting of all compounds listed in table 1 ab and Table 1c improves immune response to vaccine declined because of aging or frailty or stress or some other disease.

In other specific embodiments of the methods described above and herein, each treatment course is no longer than (a) one month, or (b) no longer than two months, or (e) no longer than 3 months. In a specific embodiment, each treatment course is no longer than (a) 5 days, (b) 7 days, (c) 10 days, (d) 14 days, or (e) 21 days. In a specific embodiment, the compound selected from the table 1 ab and Table 1c is administered every 200 day or every 30 day of each treatment course. In a specific embodiment, the treatment course is one day, two days, three days, or four days. In another specific embodiment, the compound selected from the table 1 ab and Table 1c is administered daily during each treatment course. In another specific embodiment, the non-treatment interval is at least two weeks, at least one month, at least 2 months, at least 3 months, at least 6 months, at least 9 months, or at least 1 year. In another specific embodiment, the treatment course is one In another specific embodiment, the senescence-associated disease or disorder is a cardiovascular disease selected from atherosclerosis, angina, arrhythmia, cardiomyopathy, congestive heart failure, coronary artery disease, carotid artery disease, endocarditis, coronary thrombosis, myocardial infarction, hypertension, aortic aneurysm, cardiac diastolic dysfunction, hypercholesterolemia, hyperlipidemia, mitral valve prolapsed, peripheral vascular disease, cardiac stress resistance, cardiac fibrosis, brain aneurysm, and stroke. In another specific embodiment, the senescence-associated disease or disorder is an inflammatory or autoimmune disease or disorder selected from osteoarthritis, osteoporosis, oral mucositis, inflammatory bowel disease, kyphosis, and herniated intervertebral disc. In another specific embodiment, the senescence-associated disease or disorder is a neurodegenerative disease selected from Alzheimer's disease, Parkinson's disease, Huntington's disease, dementia, mild cognitive impairment, and motor neuron dysfunction. In another specific embodiment, the senescence-associated disease or disorder is a metabolic disease selected from diabetes, diabetic ulcer, metabolic syndrome, and obesity. In another specific embodiment, the senescence-associated disease or disorder is a pulmonary disease selected from pulmonary fibrosis, chronic obstructive pulmonary disease, asthma, cystic fibrosis, emphysema, bronchiectasis, and age-related loss of pulmonary function. In another specific embodiment, the senescence-associated disease or disorder is an eye disease or disorder selected from macular degeneration, glaucoma, cataracts, presbyopia, and vision loss. In another specific embodiment, the senescence-associated disease or disorder is an age-related disorder selected from renal disease, renal failure, frailty, bearing loss, muscle fatigue, skin conditions, skin wound healing, liver fibrosis, pancreatic fibrosis, oral submucosa fibrosis, and sarcopenia. In another specific embodiment, the senescence-associated disease or disorder is a dermatological disease or disorder is selected from eczema, psoriasis, hyperpigmentation, nevi, rashes, atopic dermatitis, urticaria, diseases and disorders related to photosensitivity or photoaging, rhytides; pruritis; dysesthesia; eczematous eruptions; eosinophilic dermatosis; reactive neutrophilic dermatosis; pemphigus; pemphigoid; immunobullous dermatosis; fibrohistocytic proliferations of skin; cutaneous lymphomas; and cutaneous lupus. In another specific embodiment, the senescence-associated disease or disorder is atherosclerosis; osteoarthritis; pulmonary fibrosis; hypertension, or chronic obstructive pulmonary disease. In another specific embodiment, the compound selected from the table 1 ab and Table 1c is administered directly to an organ or tissue that comprises the senolytic cells. In another specific embodiment, the compound selected from the table 1 ab and Table 1c is combined with at least one pharmaceutically acceptable excipient to formulate a pharmaceutically acceptable composition to provide timed-release of the Compound selected from the table 1 ab and Table 1c. In another specific embodiment, the compound selected from the table 1 ab and Table 1c is administered as a bolus infusion. In another specific embodiment, the senescence-associated disease or disorder is osteoarthritis and the compound selected from the table 1 ab and Table 1c is administered directly into the osteoarthritic joint. In another specific embodiment, the compound selected from the table 1 ab and Table 1c is administered intra-articularly to the osteoarthritis joint. In another specific embodiment, the compound selected from the table 1 ab and Table 1c is administered topically, transdermally, or intradermally. In another specific embodiment, the senescence-associated disease or disorder is osteoarthritis and the compound selected from the table 1 ab and Table 1c induces production of Type II collagen in a joint. In another specific embodiment, the senescence-associated disease or disorder is osteoarthritis and the compound selected from the table 1 ab and Table 1c inhibits erosion of a proteoglycan layer in a joint. In another specific embodiment, the senescence-associated disease or disorder is osteoarthritis and the compound selected from the table 1 ab and Table 1c inhibits erosion of a bone of a joint. In another specific embodiment, pulmonary fibrosis is idiopathic pulmonary fibrosis. In another specific embodiment, the compound selected from the table 1 ab and Table 1c reduces the amount of fibrotic pulmonary tissue in the lung. In another specific embodiment, the compound selected from the table 1 ab and Table 1c is administered intranasally, by inhalation, intratracheally, or by intubation. In another specific embodiment, the senescence associated disease or disorder is atherosclerosis, and wherein the compound selected from the table 1 ab and Table 1c increases the stability of atherosclerotic plaque. In another specific embodiment, the senescence associated disease or disorder is atherosclerosis, and wherein the compound selected from the table 1 ab and Table 1c inhibits formation of atherosclerotic plaque in a blood vessel of the subject. In another specific embodiment, the senescence associated disease or disorder is atherosclerosis, and wherein the compound selected from the table 1 ab and Table 1c reduces the lipid content of an atherosclerotic plaque in a blood vessel of the subject. In another specific embodiment, the senescence associated disease or disorder is atherosclerosis, and wherein the compound selected from the table 1 ab and Table 1c increases the fibrous cap thickness of the plaque. In another specific embodiment, the senescent cells are senescent preadipocytes, senescent endothelial cells, senescent fibroblasts, senescent neurons, senescent epithelial cells, senescent mesenchymal cells, senescent smooth muscle cells, senescent macrophages, or senescent chondrocytes. In another specific embodiment, the compound selected from the table 1 ab and Table 1c kills at least 20% of the senescent cells and kills no more than 5% of non-senescent cells in an organ or tissue comprising the senescent cells associated with the senescence associated disease or disorder. In another specific embodiment, the compound selected from the table 1 ab and Table 1c kills at least 25% of the senescent cells in an organ or tissue comprising the senescent cells associated with the senescence associated disease or disorder.

In one embodiment, a method is provided for treating osteoarthritis in a subject comprising administering to the subject a therapeutically-effective amount of a small molecule compound selected from the table 1 ab and Table 1c that selectively kills senescent cells over non-senescent cells, wherein (a) the compound selected from the table 1 ab and Table 1c is administered in at least two treatment cycles wherein each treatment cycle independently comprises a treatment course of from 1 day to 3 months followed by a non-treatment interval, and wherein the non-treatment interval is at least two weeks; or (b) the compound selected from the table 1 ab and Table 1c is administered directly to the osteoarthritic joint. In another specific embodiment, the compound selected from the table 1 ab and Table 1c induces collagen Type II production in the osteoarthritic joint. In another specific embodiment, compound selected from the table 1 ab and Table 1c inhibits erosion of a proteoglycan layer in the osteoarthritic joint. In another specific embodiment, the compound selected from the table 1 ab and Table 1c inhibits erosion of a bone of the osteoarthritic joint. Also provided herein in an embodiment, is a method for inducing production of collagen Type II comprising administering to a subject in need thereof a therapeutically-effective amount of a Compound selected from the table 1 ab and Table 1c, which selectively kills senescent cells over non-senescent cells, wherein (a) the compound selected from the table 1 ab and Table 1c is administered in at least two treatment cycles wherein each treatment cycle independently comprises a treatment course of from 1 day to 3 months followed by a non-treatment interval, wherein the non-treatment interval is at least two weeks; or (b) the compound selected from the table 1 ab and Table 1c is administered directly to the osteoarthritic joint. In another specific embodiment, the compound selected from the table 1 ab and Table 1c is administered intra-articularly In another specific embodiment, the compound selected from the table 1 ab and Table 1c is administered topically, transdermally, or intradermally. In another specific embodiment, the compound selected from the table 1 ab and Table 1c is administered as a bolus infusion. In another specific embodiment, the compound selected from the table 1 ab and Table 1c is combined with at least one pharmaceutical excipient to formulate a pharmaceutical composition that provides timed release of the Compound selected from the table 1 ab and Table 1c. In another specific embodiment, the compound selected from the table 1 ab and Table 1c inhibits erosion of a proteoglycan layer in the osteoarthritic joint. In another specific embodiment, the compound selected from the table 1 ab and Table 1c inhibits erosion of a bone of the osteoarthritic joint. In another specific embodiment, the compound selected from the table 1 ab and Table 1c kills at least 20% of the senescent cells and kills no more than 5% of non-senescent cells in the osteoarthritic joint. In another specific embodiment, the compound selected from the table 1 ab and Table 1c kills at least 25% of the senescent cells in the osteoarthritic joint.

In one embodiment, a method is provided for treating a senescence-associated pulmonary disease or disorder in a subject comprising administering to the subject a therapeutically effective amount of a small molecule compound selected from the table 1 ab and Table 1c that selectively kills senescent cells over non-senescent cells, wherein the compound selected from the table 1 ab and Table 1c is administered as a monotherapy in at least two treatment cycles wherein each treatment cycle independently comprises a treatment course of from 1 day to 3 months followed by a non-treatment interval wherein the non-treatment interval is at least 2 weeks. In another specific embodiment, a method is provided for treating a senescence. associated pulmonary disease or disorder in a subject comprising administering to the subject a Compound selected from the table 1 ab and Table 1c, which compound selected from the table 1 ab and Table 1c is a small molecule compound that selectively kills senescent cells, wherein the compound selected from the table 1 ab and Table 1c is administered in at least two treatment cycles, each cycle comprising a treatment course and a non-treatment interval, and wherein the non-treatment interval is at least 2 months. In a specific embodiment, the senescence-associated pulmonary disease or disorder is pulmonary fibrosis. In another specific embodiment, pulmonary fibrosis is idiopathic pulmonary fibrosis. In another specific embodiment, the senescence-associated pulmonary disease or disorder is chronic obstructive pulmonary disease (COPD). In another specific embodiment, the senescence-associated pulmonary disease or disorder is selected from age-related loss of pulmonary function, cystic fibrosis, bronchiectasis, emphysema, and asthma. In another specific embodiment, the compound selected from the table 1 ab and Table 1c is administered directly to an affected pulmonary tissue that comprises the senescent cells. In another specific embodiment, the compound selected from the table 1 ab and Table 1c is administered by inhalation, intranasally, intratracheally, or by intubation In another specific embodiment, the compound selected from the table 1 ab and Table 1c is administered as a bolus infusion. In another specific embodiment, the compound selected from the table 1 ab and Table 1c is combined with at least one pharmaceutical excipient to formulate a pharmaceutical composition that provides timed release of the Compound selected from the table 1 ab and Table 1c. In another specific embodiment, the compound selected from the table 1 ab and Table 1c kills at least 20% of the senescent cells and kills no more than 5% of non-senescent cells in a lung of the subject. In another specific embodiment, the compound selected from the table 1 ab and Table 1c kills at least 25% of the senescent cells in a lung of the subject.

In one embodiment, a method is provided for treating a cardiovascular disease or disorder caused by or associated with arteriosclerosis in a subject comprising administering to the subject a therapeutically-effective amount of a small molecule compound selected from the table 1 ab and Table 1c that selectively kills senescent cells over non-senescent cells, wherein the compound selected from the table 1 ab and Table 1c is administered in at least two treatment cycles wherein each treatment cycle independently comprises a treatment course from 1 day to 3 months followed by a non-treatment interval, wherein the non-treatment interval is at least 2 weeks. In a specific embodiment, the subject has atherosclerosis, congestive heart failure, peripheral vascular disease, hypertension, or coronary artery disease. In another specific embodiment, the cardiovascular disease or disorder is atherosclerosis. In another specific embodiment, the compound selected from the table 1 ab and Table 1c increases the stability of atherosclerotic plaque. In another specific embodiment, the compound selected from the table 1 ab and Table 1c reduces the lipid content of an atherosclerotic plaque in a blood vessel of the subject. In another specific embodiment, the compound selected from the table 1 ab and Table 1c increases the fibrous cap thickness of the plaque. In another specific embodiment, the compound selected from the table 1 ab and Table 1c inhibits formation of atherosclerotic plaque in a blood vessel of the subject. In another specific embodiment, the likelihood of occurrence of myocardial infarction, angina, stroke, carotid thrombosis, or coronary thrombosis is reduced. In another embodiment, a method is provided for increasing the stability of atherosclerotic plaque present in a blood vessel of a subject comprising administering to the subject a therapeutically-effective amount of a small molecule compound selected from the table 1 ab and Table 1c that selectively kills senescent cells over non-senescent cells, wherein the compound selected from the table 1 ab and Table 1c is administered in at least two treatment cycles wherein each treatment cycle independently comprises a treatment course of from 1 day to 3 months followed by a non-treatment interval, wherein the non-treatment interval is at least 2 weeks. In a specific embodiment, the subject has a cardiovascular disease selected from atherosclerosis, congestive heart failure, peripheral vascular disease, hypertension, or coronary artery disease. In another specific embodiment, the cardiovascular disease or disorder is atherosclerosis. In another specific embodiment, the compound selected from the table 1 ab and Table 1c reduces the lipid content of an atherosclerotic plaque in a blood vessel of the subject. In another specific embodiment, the compound selected from the table 1 ab and Table 1c increases the fibrous cap thickness of the plaque. In another specific embodiment, the compound selected from the table 1 ab and Table 1c inhibits formation of atherosclerotic plaque in a blood vessel of the subject. In another specific embodiment, the compound selected from the table 1 ab and Table 1c reduces the amount of atherosclerotic plaque in a blood vessel of the subject. In another specific embodiment, the compound selected from the table 1 ab and Table 1c is administered parenterally or orally. In another specific embodiment, the compound selected from the table 1 ab and Table 1c is administered directly to an artery that comprises the senescent cells. In another specific embodiment, the compound selected from the table 1 ab and Table 1c is administered as a bolus infusion. In another specific embodiment, the compound selected from the table 1 ab and Table 1c is combined with at least one pharmaceutical excipient to formulate a pharmaceutical composition that provides timed release of the Compound selected from the table 1 ab and Table 1c. In another specific embodiment, the compound selected from the table 1 ab and Table 1c kills at least 20% of the senescent cells and kills no more than 5% of non-senescent cells in an arteriosclerotic artery of the subject. In another specific embodiment, the compound selected from the table 1 ab and Table 1c kills at least 25% of the senescent cells in an arteriosclerotic artery of the subject.

In certain embodiments of the methods described herein and above, the treatment course is no longer than one month or no longer than two months. In another specific embodiment, the treatment course is (a) no longer than 5 days, (b) no longer than 7 days, (c) no longer than 10 days, (d) no longer than 14 days, or (e) no longer than 21 days. In another specific embodiment, the compound selected from the table 1 ab and Table 1c is administered every 2ªd day or every 3Rd day of the treatment course. In another specific embodiment, the treatment course is one day, two days, three days, or four days. In another specific embodiment, the compound selected from the table 1 ab and Table 1c is administered daily during the treatment course. In another specific embodiment, the non-treatment interval is (a) at least one month, (b) at least 2 months, (c) at least 3 months, (d) at least 6 months, (e) at least 9 months, or (f) at least 1 year. In another specific embodiment, the treatment course is one day and the non-treatment interval is between 0.5-12 months. In other particular embodiments, when a compound selected from the table 1 ab and Table 1c is administered, the treatment course is at least 5 days. In another specific embodiment, the compound selected from the table 1 ab and Table 1c is administered as a monotherapy. In another specific embodiment, the compound selected from the table 1 ab and Table 1c is administered in three or more treatment cycles.

In other specific embodiments of the methods described above and herein, each treatment course is no longer than one month or no longer than two months. In another specific embodiment, each treatment course is (a) no longer than 5 days, is (b) no longer than 7 days, is (c) no longer than 10 days, is (d) no longer than 14 days, or is (e) no longer than 21 days. In another specific embodiment, the compound selected from the table 1 ab and Table 1c is administered every 2^{nd} day or every 3^{rd} day of the treatment course. In another specific embodiment, each treatment course is one day, two days, three days, or four days. In another specific embodiment, the compound selected from the table 1 ab and Table 1c is administered daily during the treatment course. In another specific embodiment, the non-treatment interval is at least two weeks, one month, at least 2 months, at least 6 months, at least 9 months, or at least 1 year. In another specific embodiment, the compound selected from the table 1 ab and Table 1c to the subject comprises three or more treatment cycles. In another specific embodiment, the compound selected from the table 1 ab and Table 1c is administered as a monotherapy. In another specific embodiment, the senescence-associated disease or disorder is a cardiovascular disease selected from atherosclerosis, angina, arrhythmia, cardiomyopathy, congestive heart failure, coronary artery disease, carotid artery disease, endocarditis, coronary thrombosis, myocardial infarction, hypertension, aortic aneurysm, cardiac diastolic dysfunction, hypercholesterolemia, hyperlipidemia, mitral valve prolapsed, peripheral vascular disease, cardiac stress resistance, cardiac fibrosis, brain aneurysm, and stroke. In another specific embodiment, the subject has a cardiovascular disease selected from atherosclerosis, congestive heart failure, peripheral vascular disease, hypertension, or coronary artery disease. In another specific embodiment, the senescence-associated disease or disorder is inflammatory or autoimmune disease or disorder selected from osteoarthritis, osteoporosis, oral mucositis, inflammatory bowel disease, kyphosis, and herniated intervertebral disc In another specific embodiment, the senescence-associated disease or disorder is a neurodegenerative disease selected from Alzheimer's disease, Parkinson's disease, Huntington's disease, dementia, mild cognitive impairment, and motor neuron dysfunction. In another specific embodiment, the senescence-associated disease or disorder is a metabolic disease selected from diabetes, diabetic ulcer, metabolic syndrome, and obesity. In another specific embodiment, the senescence-associated disease or disorder is a pulmonary disease selected from idiopathic pulmonary fibrosis, chronic obstructive pulmonary disease, asthma, cystic fibrosis, emphysema, bronchiectasis, and age-related loss of pulmonary function. In another specific embodiment, the senescence-associated disease or disorder is an eye disease or disorder selected from macular degeneration, glaucoma, cataracts, and vision loss. In another specific embodiment, the senescence-associated disease or disorder is an age-related disorder selected from renal disease, renal failure, frailty, hearing loss, muscle fatigue, skin conditions, skin wound healing, liver fibrosis, pancreatic fibrosis, oral submucosa fibrosis, and sarcopenia. In another specific embodiment, the senescence-associated disease or disorder is a dermatological disease or disorder is selected from eczema, psoriasis, hyperpigmentation, nevi, rashes, atopic dermatitis, urticaria, diseases and disorders related to photosensitivity or photoaging, rhytides; pruritis; dysesthesia; eczematous eruptions; eosinophilic dermatosis; reactive neutrophilic dermatosis; pemphigus; pemphigoid; immunobullous dermatosis; fibrohistocytic proliferations of skin; cutaneous lymphomas; and cutaneous lupus. In another specific embodiment, the senescence-associated disease or disorder is atherosclerosis; osteoarthritis; idiopathic pulmonary fibrosis; or chronic obstructive pulmonary disease. In another specific embodiment, n the senescence-associated disease or disorder is osteoarthritis and the compound selected from the table 1 ab and Table 1c is administered directly to the osteoarthritic joint. In another specific embodiment, the compound selected from the table 1 ab and Table 1c is administered intra-articularly to the osteoarthritic joint. In another specific embodiment, the compound selected from the table 1 ab and Table 1c is administered topically, transdermally, or intradermally. In another specific embodiment, the senescence-associated disease or disorder is osteoarthritis and the compound selected from the table 1 ab and Table 1c induces production of Type II collagen in a joint. In another specific embodiment, the senescence-associated disease or disorder is osteoarthritis and the compound selected from the table 1 ab and Table 1c inhibits erosion of a proteoglycan layer in a joint. In another specific embodiment, the senescence-associated disease or disorder is osteoarthritis and the compound selected from the table 1 ab and Table 1c inhibits erosion of a bone of a joint. In another specific embodiment, the senescence-associated disease or disorder is idiopathic pulmonary fibrosis and the compound selected from the table 1 ab and Table 1c reduces the amount of fibrotic pulmonary tissue in the lung. In another specific embodiment, the compound selected from the table 1 ab and Table 1c is administered intranasally, by inhalation, intratracheally, or by intubation. In another specific embodiment, the compound selected from the table 1 ab and Table 1c is combined with at least one pharmaceutically acceptable excipient to formulate a pharmaceutically acceptable composition to provide timed-release of the Compound selected from the table 1 ab and Table 1c. In another specific embodiment, the compound selected from the table 1 ab and Table 1c is administered as a bolus infusion. In another specific embodiment, the senescence-associated disease or disorder is atherosclerosis, and wherein the compound selected from the table 1 ab and Table 1c increases stability of atherosclerotic plaque. In another specific embodiment, the senescence-associated disease or disorder is atherosclerosis, and wherein the compound selected from the table 1 ab and Table 1c inhibits formation of atherosclerotic plaque in a blood vessel of the subject. In another specific embodiment, the senescence-associated disease or disorder is atherosclerosis, and wherein the compound selected from the table 1 ab and Table 1c reduces the lipid content of an atherosclerotic plaque in a blood vessel of the subject. In another specific embodiment, the senescence-associated disease or disorder is atherosclerosis, and wherein the compound selected from the table 1 ab and Table 1c increases the fibrous cap thickness of the plaque. In another specific embodiment, the senescence-associated disease or disorder is atherosclerosis, and wherein the likelihood of occurrence of myocardial infarction, angina, stroke, carotid thrombosis, or coronary thrombosis is reduced. In another specific embodiment, the senescent cells are senescent preadipocytes, senescent endothelial cells, senescent fibroblasts, senescent neurons, senescent epithelial cells, senescent mesenchymal cells, senescent smooth muscle cells, senescent macrophages, or senescent chondrocytes. In another specific embodiment, the compound selected from the table 1 ab and Table 1c kills at least 20% of the senescent cells and kills no more than 5% of non-senescent cells. In another specific embodiment, the compound selected from the table 1 ab and Table 1c kills at least 25% of the senescent cells.

In some embodiments of this invention, the compound selected from the table 1 ab and Table 1c is used interchangeably with the therapeutic agent reducing, inhibiting or degrading at least one of the Targets. In some embodiments of this invention, the therapeutic agent reducing, inhibiting or degrading at least one of the Targets is a small molecule. In some embodiments of this invention, the therapeutic agent reducing, inhibiting or degrading at least one of the Targets is a gene therapy.

The explanation behind the selection of compounds listed in Table 1ab, Table c and Table d.

### Table 1a

1. Train a deep neural network (DNN) according to the algorithm described in the Drug Repurposing Using Deep Embeddings of Gene Expression Profiles Yoni Donner, Stéphane Kazmierczak, and Kristen Fortney Molecular Pharmaceutics 2018 15 (10), 4314-4325
   DOI: 10.1021/acs.molpharmaceut.8b00284 or alike algorithm. e.g. described in “FC1000: Normalized gene expression changes of systematically perturbed human cells” Lonnstedt I., Nelander S., Statistical Applications in Genetics and Molecular Biology 2017 16(4), 217-242, DOI: 10.1515/sagmb-2016-0072.

It can be done like this for example (based on Donner et al., 2018):

### Data

To train the deep neural network, one can use data from the NIH Library of Integrated Network-Based Cellular Signatures Program (LINCS, http://www.lincsproject.org). Phase I and II level 4 (z-scores) data for a total of 1,674,074 samples corresponding to 44,784 perturbagens were retrieved from GEO (accession numbers GSE92742 and GSE70138). After removal of 106,499 control samples (as indicated by the sm_pert_type field), 1,567,575 samples corresponding to 44,693 perturbagens remained. Our analysis only includes the 978 L1000 landmark genes and did not use imputations. To evaluate the embedding performance, one can use functional labels from several sources. Therapeutic target classifications for 1005 drugs can be obtained from the Anatomical Therapeutic Chemical Classification System (ATC) provided by DrugCentral. There are 14, 79, 182, and 444 classes for ATC levels 1, 2, 3, and 4, respectively. Biological protein target annotations for 135 target classes and 411 drugs can be obtained from ChEMBL.

To compute structural similarities, one can use extended-connectivity fingerprints (ECFPs) and Molecular ACCess System (MACCS) keys. Tanimoto coefficients were used to compute similarity.

### Deep Neural Network

The inputs to the deep neural network are vectors of standardized L1000 expression profiles. Because L1000 measures 978 landmark genes, the vectors are 978-dimensional. Standardization is performed per gene by subtracting the mean and dividing by the standard deviation. Means and variances are estimated over the entire training set.

The calculation of the training loss for a single training example is illustrated in FIG. 1D. The embedding network is deep, self-normalizing, and densely connected. One can use dense connections with no convolutions to train deep embedding networks and observed no performance degradation even when training networks with more than 100 layers. A final fully connected layer computes the unnormalized embedding, followed by an L2-normalization layer [symbol] , where x is the unnormalized embedding, to project the embedding on the unit hypersphere.

FIG. 1D. Embedding network architecture and training loss for a single expression profile. Inputs are standardized L1000 profiles and are processed by a densely connected neural network. The output embeddings are used to predict the class (perturbagen identity) of the input by softmax where logits are cosine similarities between the profile embeddings and learned class embeddings, scaled by a learned constant. The prediction cross-entropy loss is used to train the network.

The network is trained with a modified softmax cross-entropy loss over n classes, where classes are perturbagen identities. For ease of notation, the following describes the loss for a single sample. The loss for an entire batch is defined similarly. We use L2-normalized weights with no bias term to obtain the cosines of the angles between the embedding vector and the class weights: cos θᵢ=(Wᵢ^{T}x)/(∥Wᵢ∥₂∥x∥₂), where i is the class index. The loss for a single training sample given θ={cos θᵢ}_{1≤i≤n}, where n is the number of classes (perturbagens), is

$$l ( θ ) = - log \frac{exp ( α ( cos {θ}_{i} - m ) )}{exp ( α ( cos {θ}_{i} - m ) ) + {\sum }_{\text{?}} exp ( αcos {θ}_{j} )} \qquad ( 1 )$$

$$\text{?} \text{indicates text missing or illegible when filed}$$

where i is the label (perturbagen identity), α>0 is a trainable scaling parameter, and m≥0 is a nontrainable margin parameter. During training, m is gradually increased from an initial value of 0, up to some maximum value. Inclusion of the margin forces the embeddings to be more discriminative and serves as a regularizer. A similar margin has been proposed for convolutional neural networks.

For the main experiments described here, one can use 64 hidden layers, a growth rate of 16, and an embedding size of 32. One can train the network for 8000 steps (about 2 h using a single NVIDIA K80 GPU) with a batch size of 8192, adding Gaussian noise with a standard deviation of 0.3 to the input. The margin m are linearly increased at a rate of 0.0002 per step up to a maximum 0.25. These values are chosen without a hyperparameter search and are therefore unlikely to be optimal.
2. We applied trained DNN to L1000 perturbagen profiles and obtained denoised representation of perturbagen signatures.
3. The denoised representation was used as it is described in the attached Article 1 (“Integration of genetic, transcriptomic, and clinical data reveals pharmacological modulators of human longevity”) to obtain the risk model from human genetics data which can be translated to the L1000 perturbagens expression profiles.
4. Compounds from the L1000 dataset were scored by applying the risk model from step 3. Denote compounds and its scores as LIST1.
5. Next, for each target in the ChEMBL database (ChEMBL: towards direct deposition of bioassay data. Mendez D, Gaulton A, Bento A P, Chambers J, De Veij M, Félix E, Magariños M P, Mosquera J F, Mutowo P, Nowotka M, Gordillo-Marañón M, Hunter F, Junco L, Mugumbate G, Rodriguez-Lopez M, Atkinson F, Bosc N, Radoux C J, Segura-Cabrera A, Hersey A, Leach A R.
   Nucleic Acids Res. 2019; 47 (D1): D930-D940. doi: 10.1093/nar/gkyl075 ChEMBL web services: streamlining access to drug discovery data and utilities. Davies M, Nowotka M, Papadatos G, Dedman N, Gaulton A, Atkinson F, Bellis L, Overington J P.
   Nucleic Acids Res. 2015; 43 (W1): W612-20. doi: 10.1093/nar/gkv352
   We selected compounds with activities corresponding to the pChEMBL value>=6. From these compounds find an intersection with compounds from LIST1, denote it as LIST+.
   All other compounds from LIST1 (not included in list LIST+) denote as LIST−. If the number of compounds in the LIST+ is less than 3, then exclude this target. If the number of compounds in the LIST+ more or equal 3, calculate two-sample, one-tailed Kolmogorov-Smirnov (KS) test for scores in LIST+ and LIST−. Accept target if the p-value in the KS test is below 0.01.
6. As a result of the above steps the selected targets (shown as Entrez Gene symbol) are: ABCB1, ADRA1A, ADRA1B, ADRA1D, BRD2, BRD3, BRD4, DRD2, DRD3, DRD4, DRD5, EBP, EGFR, EPHA6, EPHA8, ERBB2, HDAC1, HDAC10, HDAC11, HDAC2, HDAC3, HDAC4, HDAC5, HDAC6, HDAC7, HDAC8, HDAC9, HRH1, HSP90AA1, HTR2A, HTR2B, HTR2C, HTR5B, HTR6, HTR7, KCNH2, MAP4K5, MTOR, PIK3C2A, PIK3C2B, PIK3CA, PIK3CB, PIK3CD, PIK3CG, PIK3R1, PRKDC, SLC6A2, SLC6A3, SLC6A4 (List of Targets 1a).
7. Since MTOR is known for extending the lifespan of mammals we don't use it for the purpose of this application.
8. For the purposes of this application we selected all the compounds inhibiting any one of the targets of the List of Targets 1a with activities corresponding to the pChEMBL value>=8, according the ChEMBL database (version 27) and having a phase of clinical development at least Phase 1 and as a result of this we created compounds for Table 1a.

### Table 1B

1. We collected an age-dependent RNA-seq experimental proprietary dataset consisting of four mutant groups (daf-2 (e1370), age-1 (mg44) [at the first and second gen-erations of homozygosity], and daf-2 (e1391); daf-12 (m20) double mutant), the three examples of life-extending RNAi (daf-4, che-3 and cyc-1, representing knockdown of diverse pathways) and two independent control runs represented by C. elegans wild-type (Bristol-N2, strain DRM); 60 transcriptomes in total; mean adult lifespan extended 2.2- to 9.4-fold.
2. Then, with the help of linear gene-wise regression, we identified the transcriptomic aging signature [1], the list of genes that have statistically significant association with age rescaled by lifespan (the first list).
3. Genes in the aging signature that have positive correlation with age rescaled by lifespan are over-expressed in late life of the animals, and if a gene's correlation is negative its gene expression decreases with age.
4. Since long-lived mutant lines had 9.4-fold increased lifespan with respect to the control strains and showed the time scaling of gene expression changes—the transcriptome of long-lived mutant strains changed similar to the control strains but proportionally slower according to their lifespan.
5. The identified aging signature represents a vector of the aging-related changes of gene expression. For extracting the targets of the second list, (the PI3K-null transcriptomic signature), we corrected for age-related changes in all animals by subtracting aging-related changes in each of the strains. This was done by projecting out all gene expression changes along the aging signature.
6. After that, by gene-wise linear regression, we identified the list of genes that had statistically significant correlation with lifespan of long-lived strains. That list represents the PI3K-null signature. Genes in the PI3K-null signature that have positive correlation with lifespan are over-expressed in long-lived strains, and vise versa, genes with negative correlations have lower gene expression levels in long-lived mutants with respect to the control lines.
7. Hence, by applying the compounds that inhibit the proteins translated from the genes having positive correlation in the aging signature, or negative correlation in the PI3K-signature, we would mimic the effect opposing the aging progression, or the effects distinguishing long-lived mutants from the short-lived wild-type controls. Therefore, we would affect the aging process and either slow-down it or reverse it.
8. We combined those lists, and identified the lists of their human orthologs.
9. The inhibition of these genes or/and the proteins translated from those genes in humans will also produce life-extending effects.
10. The predictive value of the aging signature was previously experimentally tested in paper [1]. Along the lines of paper [1], to test the predictive value of the PI3K-null signature, we identified novel life-extending pharmacological interventions by comparing this signatures with gene expression profile changes in response to pharmacologic perturbations from the Connectivity Map (CMAP) database [2, 3, 16]. Using the PI3K-null signature, we predicted perturbagen-compounds opposing the transcriptomic signature distinguishing long-lived C. elegans mutant strains with a slow aging rate from short-lived strains with a fast aging rate (see Table T1a and T1b). To eliminate the confounding effect of different biological ages of nematodes in experimental cohorts, we applied the algorithm developed earlier [1]. From the top of predicted compounds, we selected 8 compounds for a follow-up lifespan screening in C. elegans assay (see Figure F1, Table T2).
11. We successfully tested the selected compounds in C. elegans survival assay (see Figure F1, Table T2). All 8 compounds produced a significant lifespan extension effect (according to the Log-rank Mantel-Cox method, all results are with p<0.05). Therefore, we experimentally validated the PI3K-null signature's pipeline for the identification of new life-extending compounds.
12. Finally by merging the transcriptomic aging signature (the first list) and the PI3K-null transcriptomic signature (the second list), we obtained the following list of targets (List of targets 1b): CDK7, CHEK2, CAPN1, CTSB, HSP90AB1, HSP90AA1, HSPA5, NFKB1, NPC1, PABPC1, ABCB11, ABCB1, PLK1, PLK3, ADRA2A, ADRA2C, ADRA2B, TUBB6, SENP8, SENP6, SENP7, MMP14, MMP13, MMP8, MMP3, MMP1, MAPKAPK2, CTSL, CTSK, CTSS, ATXN2, TBXAS1, CREBBP, PDGFRA, FLT4, FLT1, KDR, PDGFRB, FLT3, KIT, CSF1R, IKBKB, IKBKE, PGGT1B, FAAH, GFER, MAPK14, MAPK12, PRKAA1, ABCG2, ADAM17, BACE2, BACE1, TUBB6, CA7, CA2, CA1, CA5A, CA12, CA9, CA4, CTSB, FEN1, HDAC1, HDAC2, HDAC3, CBX1, SLC6A4, SLC6A2, SLC6A3, PPARD, PPARG, PPARA, PSEN2, HTR7, ADRA1B, SMN2, FYN, SRC, LCK, LYN, TARDBP, CLK2, CLK4, USP2, GBA, RECQL, BLM, WRN, CDK2, PRKAG3, TFPI, LTA4H, DPP7, BACE2, BACE1, CAPN1, CTSB, IGF1R, INSR, EYA2, HSPA5, NFKB1, TCF7L2, TARDBP, BRD4.
13. For the purposes of this application we selected from the list all the compounds inhibiting any one of the targets of the List of Targets 1b with activities corresponding to the pChEMBL value>=8, according the ChEMBL database (version 27) and having a phase of clinical development at least Phase 1 and as a result of this we created compounds for Table 1b.
14. To further validate the suggested approach for drug discovery and part of the predicted targets and compounds we tested some of the selected drugs for impact for life-span of C. elegance.

TABLE T1a
The output of CMAP tool [16] for the given PI3K-null transcriptomic
signature. Score is the CMAP score for similarity of a drug's
profile to the PI3K-null transcriptomic signature. Name is the drug
name. Description is the mechanism of action of the given drug.

| Drug name | Mechanism of action |
|---|---|
| PI-103 | PI3K inhibitor, mTOR inhibitor |
| staurosporine | PKC inhibitor, AKT inhibitor, BMX inhibitor, CDK |
| | inhibitor, CHK inhibitor, G protein coupled receptor |
| | agonist, glycogen synthase kinase inhibitor, leucine |
| | rich repeat kinase inhibitor, ribosomal protein |
| | inhibitor, sodium/hydrogen exchanger inhibitor |
| triptolide | RNA polymerase inhibitor |
| lestaurtinib | FLT3 inhibitor, growth factor receptor inhibitor, JAK inhibitor, |
| | apoptosis stimulant |
| KU-0063794 | mTOR inhibitor |
| camptothecin | topoisomerase inhibitor, beta amyloid synthesis |
| | inhibitor, hypoxia inducible factor inhibitor |
| topotecan | topoisomerase inhibitor |
| methylene-blue | guanylate cyclase inhibitor, monoamine oxidase inhibitor, nitric |
| | oxide production inhibitor, tau aggregation inhibitor |
| AG-14361 | PARP inhibitor |
| doxorubicin | topoisomerase inhibitor, DNA intercalating drug |
| NVP-BEZ235 | mTOR inhibitor, PI3K inhibitor, protein kinase inhibitor |
| PP-30 | RAF inhibitor, mTOR inhibitor |
| AKT-inhibitor-1-2 | AKT inhibitor, PI3K inhibitor |
| PHA-793887 | CDK inhibitor |
| purvalanol-a | CDK inhibitor, dual specificity tyrosine-(Y)-phosphorylation |
| | regulated kinase inhibitor |
| Merck60 | HDAC inhibitor (HDAC1, HDAC2) |
| apicidin | HDAC inhibitor |
| avrainvillamide-analog-2 | nucleophosmin inhibitor |
| CP466722 | ATM kinase inhibitor |
| BRD-K06217810 | CLK inhibitor, dual specificity tyrosine-(Y)-phosphorylation |
| | regulated kinase inhibitor, dual-specificity tyrosine-(Y)- |
| | phosphorylation regulated kinase 1B inhibitor |
| BRD-K85853281 | radical formation stimulant, RNA synthesis inhibitor, |
| | topoisomerase inhibitor |
| ISOX | HDAC inhibitor |
| MK-2206 | AKT inhibitor |
| vemurafenib | RAF inhibitor, protein kinase inhibitor |
| panobinostat | HDAC inhibitor, apoptosis stimulant, cell cycle |
| | inhibitor |
| ER-27319 | mediator release inhibitor, syk inhibitor |
| ochratoxin-a | phenylalanyl tRNA synthetase inhibitor |
| clotrimazole | cell wall synthesis inhibitor, cytochrome P450 inhibitor, |
| | imidazole inhibitor, intermediate conductance potassium channel |
| | activator, intermediate conductance potassium channel blocker, |
| | lanosterol demethylase inhibitor, sterol demethylase inhibitor |
| dorsomorphin | AMPK inhibitor, DNA damaging, TGF beta receptor inhibitor |
| ZG-10 | JNK inhibitor |
| AZD-8055 | mTOR inhibitor |
| trichostatin-a | HDAC inhibitor, CDK expression enhancer, ID1 |
| | expression inhibitor |
| JAK3-inhibitor-VI | JAK inhibitor |
| aminopurvalanol-a | CDK inhibitor, tyrosine kinase inhibitor |
| hydrocortisone | corticosteroid agonist, glucocorticoid receptor agonist, |
| | immunosuppressant, interleukin receptor antagonist |
| dasatinib | KIT inhibitor, src inhibitor, Bcr-Abl kinase inhibitor, ephrin |
| | receptor inhibitor, PDGFR tyrosine kinase receptor inhibitor, yes |
| | kinase inhibitor, Abl kinase inhibitor, Bruton's tyrosine kinase |
| | (BTK) inhibitor, discoidin domain containing receptor Inhibitor, |
| | lymphocyte specific tyrosine kinase inhibitor, tyrosine kinase |
| | inhibitor |
| ivermectin | GABA receptor agonist, acetylcholine receptor allosteric |
| | modulator, GABA release stimulant, purinergic receptor |
| | allosteric modulator |
| GDC-0941 | PI3K inhibitor |
| BRD-K37940862 | neuropeptide receptor antagonist |

TABLE T1b
The same as Table T1a, but obtained with
the older version of CMAP [2, 3].

| Drug | Type |
|---|---|
| trichostatin A | |
| vorinostat | anti-cancer |
| rottlerin | natural |
| apigenin | natural |
| trifluridine | antiviral eyedrops, combination chemotherapy |
| prochlorperazine | antipsychotic |
| sulconazole | antifungal cream |
| resveratrol | natural |
| latamoxef | antibiotic |
| tretinoin | vitamin A, acne cream, antileukotic |
| 5252917 | |
| promethazine | first-generation antihistamine (antipsychotic) |
| trazodone | antidepressant |
| procaine | =novocaine (local anesthetic) |
| imipenem | antibiotic |
| mebendazole | antihelmintic |
| LY-294002 | PI3K inhibitor |
| scriptaid | related to vorinostat (see above) |
| 0297417-0002B | |
| tonzonium bromide | surface-active compound |

TABLE T2
The summary of lifespan screening assay. The drug dose,
mean lifespan, and mean lifespan extension are presented,
along with the p-value for statistical significance of
lifespan extension (according to logrank test).

| | | | | | p value |
|---|---|---|---|---|---|
| Masked | | Dose | Mean | Mean LS | (logrank |
| name | Compound_name | (uM) | LS* (d) | ext., % | test) |
| Control | No_compounds | 0 | 22.2 | 0 | 0 |
| Compound 1 | Panobinostat | 1 | 24 | 8.1 | 0.00036 |
| Compound 2 | Entinostat | 10 | 24.1 | 8.6 | 0.00058 |
| Compound 3 | Topotecan | 5 | 24.3 | 9.7 | 0.00009 |
| Compound 4 | PI-103 | 10 | 24.1 | 8.6 | 0.00022 |
| Compound 5 | PHA-793887 | 10 | 23.7 | 6.9 | 0.00211 |
| Compound 6 | Triptolide | 10 | 24.4 | 10 | 0.00002 |
| Compound 7 | KU-0063794 | 10 | 24.5 | 10.6 | 0.00001 |
| Compound 8 | Trichostatin A | 2 | 24.4 | 10 | 0.0011 |

### Methods

### Strains

The following C. elegans strains were used in this study: wild-type strain Bristol-N2, subline DRM (herein called “N2” or “N2-DRM”); SR806 [daf-2(e1370)]; DR1694 [daf-2 (e1391); daf-12(m20)], and SR808 [age-1(mg44)] at the first (“F1”) and second (“F2”) generations of homozygosity. Strains SR806-SR808 were outcrossed 6 generations into N2-DRM; please see [5] for details. The above mutant strains, and N2-DRM, were grown in 35-mm Petri dishes, on the surface of NGM-agar (1% Bacto-Peptone, 2% agar in nematode growth medium) spotted with E. coli OP50 (a uracil-requiring mutant). Several RNAi treatments of genes (daf-4, che-3, cyc-1, cco-1, eat-4), mutation or RNAi of most of which were reported to prolong life-span, were also assessed. The animals were maintained on NGM-agar plates at 20° C., seeded with E. coli HT115 expressing double-stranded RNAs for target-gene knockdowns [5] for both RNA-preparation and lifespan studies.

### Survivals

Lifespan assays were conducted at 20° C., as described previously [5]. Briefly, synchronous cultures were initiated by lysis of gravid hermaphrodites in alkaline hypochlorite. Worms were selected at the L4 larval stage, placed 50 worms per plate, and transferred at 1- to 2-day intervals onto fresh plates during days 1-7, and at 2- to 3-day intervals after that. A worm was scored as dead if it failed to move, either spontaneously or in response to a mechanical stimulus; lost worms were excluded (censored) from the survival analysis.

Our survival study confirms longevity of the worm strains subjected to the treatments targeting genes known to affect aging in previous studies. The relative lifespan modification effects in some cases proved to be somewhat smaller, which can probably be attributed to the use of mutation instead of RNAI or a different developmental temperature in the original studies, or neuronal resistance to RNAi, which may be required for full life extension.

We note that the SR806/SR808 survivals and their controls from Table T1 were run quite a few years ago [5,6], and while all results with those strains were consistent over a 4-5 year period preceding publication, the adult lifespans have not been repeated recently under our current lab conditions. The control lifespans for DR1694 were anomalously long, but still indicate a roughly 2.5-fold life extension by the double mutant. We are currently planning additional experiments to see if it is the bacterial stock we use to feed “normal” controls, but it deserves an independent study and is left for future research.

Drugs were prepared in small volumes (60-100 μl per 10-cm plate), at levels calculated to achieve the indicated concentrations upon equilibration with the full agar-medium volume. Plates were overlaid with drug solutions and rocked with rotation as liquid was absorbed into agar, 24 h prior to use. Worms were transferred to fresh drug-equilibrated plates daily for 12 days and after that, on alternate days (M-W-F).

### RNA Isolation

Synchronized strains of C. elegans were grown on 100-mm NGM plates, as above, and harvested for RNA extraction at the ages indicated. Worms were washed off plates and rinsed twice in survival buffer; after 30 min at 20° C. (to allow digestion of enteral bacteria), they were flash frozen and stored at −80° C. Frozen worms were ground in a dry-ice-cooled mortar and pestle, and total RNA was extracted using RNeasy RNA extraction kits (Qiagen), followed by RNA purification for construction of transcript libraries using TruSeq RNA kits (Illumina, v.2). Sequences are generated as PE100 multiplexes, 100-bp paired-end reads from an Illumina HiSeq2500 or NextSeq instrument, producing 40-50×106 reads per sample. Paired samples are analyzed with DESeq2 (v1.4.5), and combined sequences are mapped to the C. elegans genome using TopHat [7].

### Experimental RNA-Seq Dataset

RNA-seq reads were mapped to the C. elegans genome (WBcel235, Ensembl annotation) using TopHat 2.1.1 (with-b2-very-sensitive and -GTF options) [7] and gene-level read counts were obtained using the htseq-count software [8]. Low-expressed genes with at least one zero read count per sample were removed from subsequent analysis. Raw read counts were normalized using the upper quartile method [9] and converted to RPKM values using the edgeR library [10].

### Preparation of the Signature of Aging for the Connectivity Map Screening

To transform the list of genes associated with aging in C. elegans into the form appropriate for the Connectivity Map (CMAP) database [2,3,16], we first identified human orthologs for the genes from this list using OrthoList database comprising information from four other databases: Ensembl Compara [12], InParanoid [13], NCBI HomoIoGene Database, OrthoMCL [14]. Since, CMAP requires human genes to be presented by HG-U133A tags (Affymetrix Human Genome U133A Array), the g: Profiler database [15] was used to map human Ensembl gene IDs to HG-U133A tags. Finally, the lists of up- and down-differentially expressed with age genes were formed and used to predict the list of prospective drugs-perturbagens using CMAP. These drugs are expected to reverse the gene-expression profiles to a younger state.

### Aggregation Test

The aggregation tests were done in the AM141 strain, a model of huntingin-like aggregation in which a tract of 40 glutamines is fused in frame to YFP. These worms have only diffuse YFP fluorescence as larvae, but as adults progressively accrue punctate aggregates over about 6 days. Drugs are usually introduced just before the start of adulthood (late L4 stage). We take pictures of the fluorescent signal and plot either aggregate count (using imageJ) or total YFP intensity within foci.

### Table 1ab

Table 1ab consists of all compounds from the Table 1a and all compounds from the Table 1b created as shown above and the just one of the each repetitious compounds is left in the table 1ab

We also added the wording about other inhibitors of any one of the targets from Target list 1a and Target list 1b which have anti-aging properties.

### Table 1c

We selected all the compounds inhibiting any one of the targets of the List of Targets 1a and List of Targets 1b with activities corresponding to the pChEMBL value>=6 according the ChEMBL database (version 27) and related to IBScreen library.

We also added the wording about analogs of compounds from TABLE 1c will have anti-aging properties.

### REFERENCES

[1] Tarkhov, A. E., Alla, R., Ayyadevara, S. et al. A universal transcriptomic signature of age reveals the temporal scaling of Caenorhabditis elegans aging trajectories. Sci Rep 9, 7368 (2019). https://doi.org/10.1038/s41598-019-43075z
[2] Lamb, Justin, Emily D. Crawford, David Peck, Joshua W. Modell, Irene C. Blat, Matthew J. Wrobel, Jim Lerner et al. “The Connectivity Map: using gene-expression signatures to connect small molecules, genes, and disease.” science 313, no. 5795 (2006): 1929-1935.
[3] Lamb, Justin. “The Connectivity Map: a new tool for biomedical research.” Nature reviews cancer 7, no. 1 (2007): 54.
[4] Kamath, R. S. & Ahringer, J. Genome-wide RNAi screening in Caenorhabditis elegans. Methods 30, 313-321 (2003).
[5] Ayyadevara, S., Alla, R., Thaden, J. J. & Shmookler Reis, R. J. Remarkable longevity and stress resistance of nematode PI3K-null mutants. Aging Cell 7, 13-22 (2008).
[6] Shmookler Reis, R. J. et al. Modulation of lipid biosynthesis contributes to stress resistance and longevity of C. elegans mutants. Aging (Albany NY) 3, 125-147 (2011).
[7] Kim, D. et al. TopHat2: accurate alignment of transcriptomes in the presence of insertions, deletions and gene fusions. Genome Biol. 14, R36 (2013).
[8] Anders, S., Pyl, P. T. & Huber, W. HTSeq, a Python framework to work with high-throughput sequencing data. Bioinformatics 31, 166-169 (2015).
[9] Bullard, J. H., Purdom, E., Hansen, K. D. & Dudoit, S. Evaluation of statistical methods for normalization and differential expression in mRNA-Seq experiments. BMC Bioinformatics 11, 94 (2010).
[10] Robinson, M. D., McCarthy, D. J. & Smyth, G. K. edgeR: a Bioconductor package for differential expression analysis of digital gene expression data. Bioinformatics 26, 139-140 (2010).
[11] Shaye, D. D. & Greenwald, I. Ortholist: a compendium of C. elegans genes with human orthologs. PloS ONE 6, e20085 (2011).
[12] Aken, B. L. et al. The Ensembl gene annotation system. Database 2016, baw093 (2016).
[13] Sonnhammer, E. L. & Östlund, G. Inparanoid 8: orthology analysis between 273 proteomes, mostly eukaryotic. Nucleic Acids Research 43, D234-D239 (2015).
[14] Li, L., Stoeckert, C. J. & Roos, D. S. OrthoMCL: identification of ortholog groups for eukaryotic genomes. Genome Res. 13, 2178-2189 (2003).
[15] Reimand, J. et al. g: Profiler—a web server for functional interpretation of gene lists (2016 update). Nucleic Acids Research 44, W83-W89 (2016).
[16] Subramanian, Aravind, et al. “A next generation connectivity map: L1000 platform and the first 1,000,000 profiles.” Cell 171.6 (2017): 1437-1452

### Article 1

### Article 1 (“Integration of Genetic, Transcriptomic, and Clinical Data Reveals Pharmacological Modulators of Human Longevity”)

### Introduction

The cost of bringing a new drug to market has, until very recently, been increasing exponentially, as described by Eroom's law [1]. Improvements in recent years have been mostly due to increasing reliance on genetic evidence for new therapeutic targets [1]. Overall, 73% of the projects on drug discovery that rely on a genetic link between target and disease are successful in Phase II trials, compared with only 43% of the projects without such support [2]. Moreover, the proportion of drugs with the direct genetic support increased along the development pipeline from 2% at the preclinical stage to 8.2% among the approved drugs, especially when the genes are predicted to be causal [3], [4]. What benefits the research on the genetics/drug discovery most is the fact that Genome-Wide Association Studies (GWAS) enable the identification of disease risk loci without a priori hypotheses [5].

Recent studies involving whole-exome sequencing (WES) data from UK Biobank (UKB) associated loss of function (LOF) burden with 1,741 phenotypes, including disease-associated LOF variants [6]. A survey of LOF variants in humans predicted 100 LOF variants per individual, including 20 genes that carry homozygous LOFs and hence are likely to be non-functional [7]. We also observed that the burden of protein-truncating variants (PTVs) is negatively associated with both remaining lifespan (all-cause mortality) and healthspan (disease-free survival) in UKB [8].

On the technical level, WES provides an extremely rich signal, comprising 39 megabase pairs and 4,735,722 variants within targeted regions [6]. At this number of variables, any attempt to produce a multivariate model for an association with phenotype will most probably fail, and the single SNP association testing may be underpowered due to the missing effects from other SNPs [9]. Feature selection (or dimensionality reduction) is required to empower genetic studies. It can be achieved by aggregating gene variants into biologically relevant gene sets or pathways [10]. Pathway definitions, however, can be incomplete. We hypothesized that genetic variants can be aggregated into pathways without a priori information by deep learning of large-scale gene expression datasets.

In this work, we developed such an approach, taking advantage of the LINCS L1000 [11] and UKB datasets. We applied a deep neural network to de-noise and compress the transcriptomes into low-dimensional representation (embedding) of gene expression. In vitro gene knockouts provide the gene expression response of cells to genetic perturbations and therefore can be used to model gene expression produced by germline LOF mutations in vivo. We thus predicted pathway activation due to germline PTV burden for each UKB subject and then constructed a mortality model using WES and the death register from UKB. We further trained this model and employed it to discover the transcriptomic signature of longevity and scored small molecules from LINCS L1000 according to their predicted effects on human lifespan. Our predictions were validated by using the largest dataset of screenings for longevity drugs in Caenorhabditis elegans [12]. Finally, we predicted molecular pathways and genes with the most genetic support that can be used to develop life-extending interventions in humans.

### Results

### Deep Neural Network for Batch Removal in the LINCS L1000 Dataset

The LINCS L1000 dataset is a unique resource for associating genetic and pharmacological perturbations with gene expression patterns, which is a key feature of our current study. However, the dataset suffers from non-biological variance effects and the need for dimensionality reduction. To address these issues, we utilized a deep neural network (DNN) with an architecture similar to that described in [13]. First, we generated compressed 10-dimensional representations (embeddings) of the 978-dimensional differential gene expression signatures in LINCS L1000 data processing at level 4 [11]. The reduced feature sets were then used for batch removal by classifying biologically identical perturbations in multiple repetitive experiments. Overall, the network was trained using over 1.2M cell culture samples from ˜ 80 cell lines and all molecular and genetic perturbations including more than 7 measurements each.

To obtain a compressed representation of biologically different perturbations, we computed consensus profiles (signatures) treating different doses of the same molecular perturbation as separate biological conditions. The procedure involved averaging over all available repeats including measurements of the same perturbation in different cell lines and times points. Most of the transcriptomic signatures of small molecule perturbations in LINCS L1000 were only available at a very high 10 μM concentration and hence, for consistency, only the measurements at this concentration were used in the calculations below.

We further analyzed gene knockdowns by shRNA in LINCS L1000, since this dataset covers the largest set of gene targets. Altogether, we utilized 17,902 transcriptomic profiles of cells subjected to shRNAs that individually suppress 4,308 genes, including 939 genes corresponding to the transcripts directly measured by the L1000 platform (so-called landmark genes). Aggregation for shRNA profiles was performed over all replicas with knockdowns of the same gene, i.e. different seed sequences, cell lines, and time duration points were collapsed during averaging.

### Mortality Model in UKB and Genetic Signature of Longevity

We hypothesized that gene expression profiles in response to genetic (shRNA) perturbations in the LINCS L1000 dataset can be used to model the effects of LOF variants on gene expression (see FIG. 1 for a summary of the data used and overview of the computational pipeline). The latest update of UKB includes WES data from 200,458 participants, including 90,079 (44.94%) males of mean age 57.6 years and 110,379 (55.06%) females of mean age 56.6 years. In this cohort, 11,279 subjects died during the follow-up period of 14.5 years (2005-2020).

We annotated population-level exome OQFE variants [6] with SNPeff software [15] and identified loss-of-function (LOF) variants using the method from [16], implemented in SNPeff. We found 3,534 genes on chromosomes 1-22 which matched shRNA knockdown data from the denoised L1000 dataset. There were about 26 predicted LoF genes per subject with the standard deviation of 3.75. We employed UKB genotypes to build the matrix LOFⁿ_{g} of binary variables based on the presence of each LOF in a gene g of subject n.

Both the risk of death and the incidence of major age-related diseases increase exponentially as a function of chronological age during adult life. Hence, to associate the effects of LOFs on lifespan, one would have to train a full multivariate all-cause-mortality risk model, such as a Cox proportional hazards model, in the form:

$$log {M}^{n} = {Γ {t}^{n} + γ {sex}^{n} + … + {}^{X} {β}_{g} {LOF}_{g}^{n} ,}_{g} \qquad ( 1 )$$

where Mⁿ is the risk predictor for a patient n, tⁿ and sexⁿ are the age at the time of the assessment and gender, respectively. Here, the dots stand for other covariates, such as regional affiliations/assessment centers, ethnic variables, and principal components (PCs) of UKB genotypes [17]. The coefficients Γ, γ, and β_{g} are the Cox regression coefficients characterizing the effects of age, sex, and loss of a gene g.

As the total number of features in a genotype or even the overall number of genes is very large, the direct solution of the full regression problem (1) is unfeasible. To produce a tractable and numerically stable solution we followed the two-step procedure. First, we assumed that the effect of LOF variants is small and solved a proportional Cox-Gompertz hazards model (1) while neglecting the effects of LOF variants altogether (all beta_g=0).

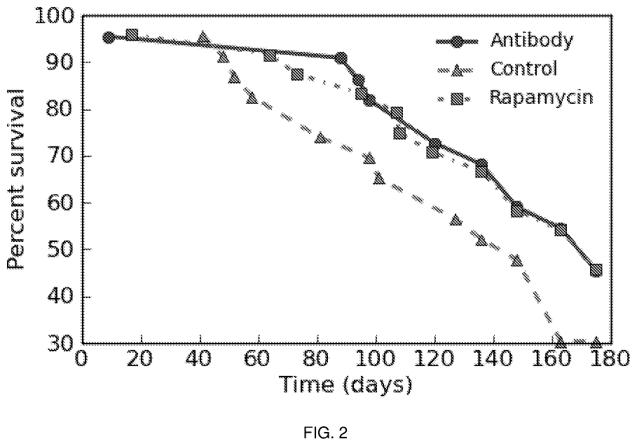

The parameters of the first-order death risk model are shown in FIG. 2. The log-linear regression coefficient for the chronological age in the lifespan model was 0.106 per year, which is consistent with a typical estimate of the Gompertz slope parameter in the range of 0.06-0.11 per year [18]. We found that mortality was higher in males than in females (HR=1.6, p=2e−136). This risk ratio is equivalent to an approximately five years difference in lifespan and is in agreement with the known effect of sex on longevity (XXX refs). The model produced increased mortality estimates associated with smoking, Townsend deprivation index, and increased BMI. We also noted significant effects on mortality risks associated with clinical assessment centers (most notably, the increased risks associated with residing in Edinburgh and Glasgow) and the genetic ancestry variables (PC3, PC4, and PC13).

Following the xxx [8], we validated the effects of LOF burden on longevity in UKB in increasingly larger cohorts. This corresponds to a simplified solution of Eq. (1), which can be obtained assuming that the coefficients β_{g} are the same for every gene. Accordingly, we produced a series of Cox proportional hazards models for the age at the assessment, sex, the first 10 genetic PC scores, and the LOF burden aggregated in bins of increasing MAF (FIG. 7). We found that the significance of the association between the LOF burden and survival increased with the number of samples at any level of MAF. Less frequent damaging variants had consistently stronger effects on longevity. Henceforth we included only LOFs with MAF<1e-3 in subsequent calculations. Of those gene variants, stop codon gained and frameshift variants were responsible for most of the effect at any level of MAF (FIG. 8).

The effect of LOF variants on mortality is small and hence genetic factors may affect both the rate of aging Gamma and the age-independent mortality. The determination of small effects in both quantities at the same time is a mathematically ill procedure due to the degeneracy of Gompertz fit\cite{ }. Accordingly, we fixed Gamma and employed a form or perturbation theory using the first-order death risk model as the baseline (see \cite{ } and MM). The procedure is equivalent to a regression of the genetic variables onto the Martingale residuals of the Cox-Gompertz fit and henceforth is referred to as Martingale Residuals Regression (MRR).

We used sequential (independent) LOF variants MRR screen (FIG. 9) involving the age at the assessment, sex, and the first 10 genetic PC scores in the baseline model. Remarkably, there were two individual gene variants reaching the whole genome significance level: BRCA2 (HR=1.1, p=6e-12) and ASXL1 (HR=1.14, p=1.7e-7).

The complete solution of (1) can be obtained by noting that phenotypic variation should depend on the gene expression patterns, which may be organized into a small number of pathways. Accordingly, we regularized the otherwise intractable regression problem by changing the variables

$${{β}_{g}}_{α} = X {β}_{α} Δ {PA}_{α g} \qquad ( 2 )$$

where α enumerates molecular pathways, PAα are the pathway activations (the normal variables of the underlying molecular interaction network) and ΔPAαg is pathway activation in response to the loss of gene g. Accordingly, βα=d(log Mn)/dPAα are the log-linear risk model coefficients, associated with activation of a pathway a. The expectation is that the total number of meaningful pathways and hence the number of the regression coeffi
of the dimensionality reduction technique proposed here. It cannot be computed and may only be obtained from an experiment. We, therefore, employed the transcriptomic signatures of shRNA knockdowns and CRISPR gene knockouts from LINCS L1000 as the necessary proxies. Since the pathway activations are approximately related to the latent variables of our network by a linear transformation (such as PCA), we used the embeddings as proxies for pathway activations PA_{α}. Accordingly, we used Eq. (2) and transformed the individual genetic profiles PTVⁿ_{g} of every UKB subject in Eq. 1 into the pathway activation due to life-long effects of germline mutations:

$${PA}_{α}^{n} = {\sum }_{g} Δ {PA}_{g}^{α} {PTV}_{g}^{n} ,$$

where the summation occurs over all the genes from the LINCS L1000 shRNA knockdowns subset. The pathway activation scores are representations of the transcriptomic shifts due to the germline PTV mutations and can be used as additional covariates in the multivariate proportional hazards model.

The transcriptomic shifts PAⁿ_{α} can be used instead of the genetic profiles PTVⁿ_{g} in the Cox-proportional hazard model in Eq. 1. We performed the estimation of the parameters using MRR of the pathway activation scores (PAs). The genetic component (the linear combination of the pathway activation scores, GLSⁿ=−Σ_{α}β_{α}PA_{α}ⁿ, of the risk model is hereinafter referred to as “the genetic longevity score” or GLS. The sign convention is such that larger GLS values correspond to the lower risk and hence predict longevity. GLS was significantly associated with mortality in the whole dataset according to the log-likelihood ratio test (HR=1.04 at one std of GLS level, p=0.003, see Materials and Methods for details of the calculation). We also cross-validated GLS by training the MRR model in half of the dataset and testing the association of GLS with mortality in the other half of the data (p=0.008 and p=0.02, respectively).

To understand the biology behind the genetic longevity score, we identified gene transcripts associated with the longevity phenotype. For this, we used full transcriptomes from LINCS (12K gene transcript each) and ran the correlation between gene transcript levels and the genetic longevity score computed for the same samples using the gene expression profile embeddings.

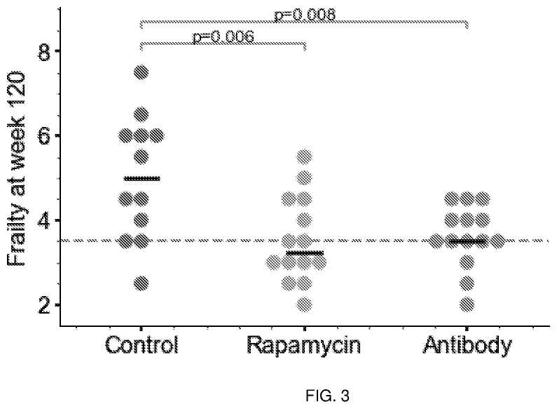

We used the correlation coefficients as the ranked list for GSEA by WebGestalt and obtained the list of over- and under-represented categories, associated with GLS (FIGS. 3a and 3b for GO Biological Process categories and KEGG pathways, respectively). Genes with a positive correlation with GLS were enriched for stress response, lipid metabolism, transition metal homeostasis and other pathways. Genes with a negative correlation with GLS were enriched in DNA replication and repair, ribosome biogenesis pathways, TCA cycle and other pathways. These findings suggest that the identified pathways are important for the regulation of human lifespan.

### Scoring Molecular Perturbations Against Aging

The mortality risk model prediction or GLS can be performed for every molecular or genetic perturbation from LINCS L1000. This can be done, since the DNN can translate any L1000 sample into PA scores, which, in turn, can be used to compute the longevity score, corresponding to the negative risk associated with the drugs. The values can be used to rank-order drugs or genetic interventions according to the expected life-extending effect (the higher expected effect is associated with the larger value of the score).

To validate the GLS model, we used the predictions against the results of a large experimental study of life-extending properties of drugs in Caenorhabditis elegans [12]. This experiment measured lifespan in the presence of 1,274 drug compounds from the Library of Pharmacologically Active Compounds (LOPAC). Of these, 708 drugs were included in the LINCS L1000 library and hence could be directly processed by our computational pipeline. The rank-order of the LOPAC compounds established by our model did not convincingly favor the compounds that extended C. elegans lifespan in the experiment, see LS⁺ ROC AUC in FIG. 5 (p=0.16, 39 positive cases; see notes on the significance evaluation in Materials and Methods). The lack of the association of GLS and the pro- and anti-longevity properties of the compounds required further investigation. One explanation is species difference, as our model was built on human data, but validated in C. elegans. It is also possible that the predicted score of molecular perturbations may be dominated by toxic off-target effects due to high doses. To disentangle the effect of inhibition of a particular target and the multitude of off-target effects, we followed the statistical procedure suggested in [?]. Thus, we grouped compounds according to their claimed Mechanism of Action (MOA, as listed in ChEMBL database [20]). This procedure revealed the difference between the lifespan scores of compounds with the same target vs. the rest of the compounds and reported statistically significant associations of the targets and the lifespan score (Table 1). The advantage of the procedure is that it can be used to rank all drugs from an experiment [12] as soon as there is information regarding drug targets irrespective of the availability of relevant transcriptomes in

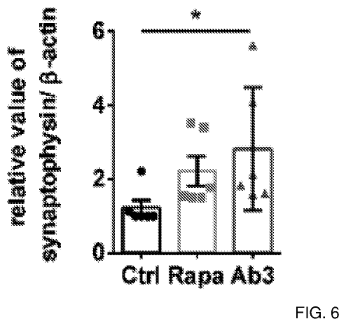

LINCS L1000. To quantify the effect of drugs, we used the mean effect (GLS) associated with the ChEMBL targets of compounds (FIG. 6). This procedure requires a known target (or targets) of a drug. We were able to link ChEMBL targets for 465 of the 1274 LOPAC compounds. This time, the best-scored compounds significantly enriched the list of experimental life-extending compounds (p=0.002, 38 positive cases) and failed to enrich the list of the drugs with the life-shortening effect (p=0.43, 33 “positive” cases).

Some of the predicted longevity targets are the targets of chemotherapies and hence we decided to address the relation of GLS to cell toxicity. We used the cell viability data from [ ] to build a model, using our gene expression representations (the embeddings from DNN) to predict cell viability. The model was a log-linear (logistic) classifier trained to predict the reported loss of cell viability in at least one of the cell lines. The inferred toxicity score, that is the log-odds ratio of the toxicity prediction model, could be computed for every molecular perturbation from LINCS L1000 and exhibited no meaningful correlation with GLS

Finally, we examined if the deep neural network architecture employed here provides a superior performance relative to the state-of-the-art batch removal techniques. We tested our pipeline using the raw L1000 gene transcripts levels and the de-noised LINCS L1000 representations of the same samples from the Fc1000 dataset [ ]. This work suggested a SVD-based batch-removal method to build a linear transformation of the raw L1000 gene expressions into the vector of the same size (1000). The publicly available Fc1000 dataset also provides the transformed representations of shRNA and drug perturbations and, therefore, can be used to train the all-cause mortality model from UKB data.

Neither of the models trained from the raw L1000 or Fc1000 transformed datasets were able to produce a significant enrichment from the list of life-extending and -reducing compounds from [12] using the direct compound scores (FIGS. 10 and 12, respectively). Target-deconvolution universally improved the results (FIGS. 11 and 13, respectively), and calculations in the raw and Fc1000 datasets led to significant enrichment of life-extending compounds from the experiment.

To compare the noise reduction performance of our network and Fc1000 in the most fair way, we performed the target convolution procedure from [ ] using the MoA of drugs from STITCH database as in [21]. The Fc1000-based analysis produced less enrichment (ROC AUC of 0.63 vs. 0.72, see FIGS. 13 and 6, respectively) and returned fewer (30 vs. 36, not shown) targets associated with human longevity than our model. The Fc1000-based GLS score also negatively and significantly enriched the list of drugs reducing the lifespan of nematodes in the experiment.

### Discussion

GWAS has been criticized for the interpretability of its findings and the small effect sizes of most risk variants. Approximately 90% of the SNPs found by GWAS are located in non-coding regions, suggesting that they may alter transcription of one or more target genes by modifying splicing, non-coding RNAs, or transcription factor (TF) binding sites \cite{gallagher2018post, schaub2012linking, maurano2012systematic}. The size of the new biological information in WES, and upcoming Whole Genome Sequencing (WGS), without a proper regularization, places even higher requirements on the number of samples required for successful application of even the simplest linear models.

We hypothesized that the analysis of large-scale transcriptomic datasets from genetically manipulated cell lines may be employed for feature extraction, dimensionality reduction, and hence regularization in genetic studies. This is possible, in principle, since tens of thousands of experiments in multiple repeats should allow to probe the underlying regulatory network interactions and infer correlated molecular patterns (pathways). The genetic regulatory networks produce highly reliable molecular level patterns in response to perturbations. Therefore, gene expression and other physiological data is redundant, and multiple genes regulate the same pathways. That is why we should not be perplexed when the genes usually associated with disease in GWAS are often different from those currently used as targets for pharmacological interventions in animal models and clinical trials.

As a proof of concept, we choose aging, an archetypal example of a complex disease involving life-long changes resulting in progressive loss of resilience and death. Most of the current mechanistic insights into the basic biology of aging originate from studies of lab animals, such as nematodes [ ], fruit flies [ ] or mice [ ]. Mice is currently the predominant preclinical model of aging and age-related diseases, but it is not universally applicable, e.g. mice are not champions of longevity and, unlike humans, they mostly die of cancer [ref]. Therefore, it is an open question as to what the best strategy is when it comes to the identification of anti-aging interventions for successful therapeutic application in humans.

GWAS of human lifespan, including examining its proxies, such as extreme lifespan, parental survival, and healthspan, produced a number of gene variants potentially associated with human aging. GWAS on centenarians consistently associate loci near APOE gene with extreme longevity, and, depending on population, loci near FOXO3A, HLA-DQA1 and SH2B3˜\cite{melzer2019}. GWAS for several longevity proxies, such as parental lifespan˜\cite{Pilling2017} and healthspan˜\cite{zenin2018identification}, confirmed most of the longevity variants and identified additional ones.

Alternatively, large collections of transcriptomes, such as LINCS and L1000, or its earlier version CMAP, have been used extensively for in silico drug repurposing in general and specifically in aging\cite{ }. One possible approach would be to look for drugs capable of reverting the gene expression signature associated with age in human tissues˜\cite{donertacs2018gene}. Similar results were obtained and later experimentally confirmed in \textit{C. elegans}˜\cite{janssens2019transcriptomics}. Aside from well-known life-extending compounds, such as mTOR and PI3K inhibitors, the experiments favored HDAC and HSP90. Recently, HSP90 inhibitors demonstrated senolytic properties˜\cite{ }. However, this approach also has problems in that age-related changes include, in addition to damaging changes, adaptive changes, and their reversals by interventions may lead to unwanted consequences.

GWAS could be used to predict drug targets for diseases˜\cite{lau2020turning}. However, the effects on expression remain unknown for most of the variants. One attractive possibility is to link SNPs to gene expression using eQTL˜\cite{ }. Unfortunately, the largest eQTL dataset, GTEx, is still too small (the dataset contains samples from 49 tissues of approximately 900 subjects), and is biased towards the European population ($67\% $) [ref]. Therefore, only a limited number of frequent SNPs can be associated with gene expression. The limitations in statistical power are even more obvious now, as it turns out that the total number of the most certainly damaging mutations in WES is associated with diseases˜\cite{ganna2018quantifying}, and with lifespan and healthspan˜\cite{Shindyapina2020}.

The following comments are related to the interpretation of the model. The genetic longevity score, GLS, is a combined effect of a multitude of ultra-rare naturally occurring germline mutations. The effect of GLS on mortality and hence the lifespan was not small (HR=1.04), which is comparable to the effect of the genetic locus near APOE (HR=1.05 [Joschi]) or LOF in BRCA2 or ASXI1 in this study (HR˜1.1). Hence the integration of LOF on a pathway level exemplified in this study led to the additional explained variance (0.5 years) of lifespan on par with other established approaches.

The GLS can be computed for every perturbation and works very well in differentiating between the life-extending and shortening compounds in the experiment˜\cite{ye2013pharmacological} (Fig.˜\ref{fig:ci_genetic_Is}). There could be two explanations of these observations: a technical one and the one of more principal importance. First, according to to˜\cite{ye2013pharmacological}, the life-extending compounds in the experiment were measured twice, in the primary and confirmatory screens. In contrast, the compounds reducing lifespan were tested only once. Therefore, the list of life-extending compounds may be more reliable in principle. We also observed all possible combinations of the effect signs for known anti-aging and toxic compounds, and obtained positive enrichment (ROC AUC>0.5) for both life-extending and life-shortening compounds in Fig.˜\ref{fig:ls_deconv}.

We believe that the gene expression profiles of most drugs in LINCS L1000 are dominated by off-target effects due to high concentration of drugs used ($10\mu M$). Such conditions are hardly achievable pharmacologically and hence clinically irrelevant. To reveal the impact of inhibition of specific protein targets, we applied the target deconvolution technique, inspired by TargetScan ˜\cite{ }. We assumed that the off-target effects are random and therefore sought systematic association of the longevity score with molecular perturbagen targets.

The statistical procedure involved grouping of molecular perturbations from LINCS L1000 by their reported ChEMBL targets (direct binding at pKd levels exceeding the cutoff of $7$ or $K_d<100 nM$). Subsequently, we made group-comparison of the genetic longevity scores distributions of drugs hitting a specific target with all other molecules in the dataset and reported the targets, associated with the largest and most significant effects. Target deconvolution identified the longevity effects associated with the inhibition of a protein target. It also improved the performance of GLS, so it outperformed the direct score in the C. elegans dataset˜\cite{ye2013pharmacological}.

We believe that gene expression profiles of drugs obtained at a very high-concentration should be taken with a grain of salt. On average, in tests involving multiple drugs with the same effect, the conclusions from the transcriptomic studies can still be reliable if the effects of drugs are aggregated at the target level.

Target deconvolution may be more reliable but has the obvious drawback. The procedure may only work for the drug targets (MOAs) with a sufficient number of measurements in LINCS L1000 and ChEMBL. By the very nature of the method, there is a bias towards well-studied molecular targets. The choice of LOPAC library in the experiment also suffers from a similar bias: pharmacologically active compounds reflect GPCR modulators' over-representation, comprising XXX\% of drugs sold in pharmacies.

The gene expression levels measured in LINCS L1000 are dominated by batch effects. Thus, it should not be surprising, that training of the mortality risk model in the raw LINCS L1000 gene expressions fails to pick up sufficient signal to noise ratio in the Cox proportional hazards model to produce a significant enrichment of positive and negative hits in the experiment˜\cite{ye2013pharmacological}, Fig.˜\ref{fig:ls_deconv_rawdirect} and \ref{fig:ls_deconv_rawTS}.

Our batch removal algorithm is loosely related to SVD batch removal techniques, such as RUV. To provide a baseline, we trained the survival model using the denoised representations of LINCS L1000 samples (summarily available for download and usage as Fc1000) and produced no significant associations between the predicted longevity scores and the life-extending properties of the compounds (neither by scoring the drugs directly or after target deconvolution using STITCH database, see Figs.˜\ref {fig:ls_deconv_Fc1000direct} and \ref{fig:ls_deconv_Fc1000TS}). The target deconvolution in Fc1000 produced much fewer hits' with a notable overlap with our DNN output (e.g. SLC6A2 and HTR6).

From the biological standpoint, our findings support the empirically established evolutionary conservation of aging pathways. Gene set enrichment of the genes associated with human longevity in UKB revealed DNA repair, replication, stress response, and ribosome biogenesis as relevant pathways, all typically discussed in the context of longevity in model organisms˜\cite{ }. Specifically, reduced translation and ribosome biogenesis are associated with a longer lifespan in model organisms and were negatively associated with longevity in our analysis of human data. The findings suggest that these pathways are evolutionarily conserved targets of aging. Our model further predicted mTOR inhibitors as promising anti-aging drugs. mTOR regulates translation and is the most validated target of aging in model organisms (refs). Most notably, mTOR inhibitor rapamycin extends the lifespan of genetically heterogeneous mice (ref). However, to our knowledge, mTOR has never been identified as a longevity target in a human genetic study. We thus expect that our findings are biologically relevant and may be successfully translated to extend lifespan in humans.

Inhibitors of histone deacetylases (HDACs) represent another prominent example. (Table 1)

Epigenetic drugs including HDAC inhibitors extended lifespan of model organisms, including C. elegans˜\cite{edwards2014d, evason2008valproic}, Drosophila melanogaster˜\cite{mcdonald2013chemical, zhao2005lifespan}, and mouse models of accelerated aging˜\cite{krishnan2011histone}. Mechanistically, the drugs revert the age-related alteration in histone acetylation that in turn activated expression of pro-longevity transcription factors in mice models of age-related diseases˜\cite{mcintyre2019molecular}. Pro-longevity effects of HDAC inhibitors in model organisms are now supported by our analysis of human genetics. Since the drugs are already used in neurology and cancer treatments, we propose a further clinical investigation of the HDAC inhibitors against aging and frailty.

A significant number of top-scoring drugs and targets are linked to serotonin and dopamine signaling. Although serotonin modifying drugs are widely used in psychiatry and neurology, the effect of serotonin is not limited to CNS, it regulates numerous biological processes outside the brain including cardiovascular, gastrointestinal and endocrine function˜\cite{berger2009expanded}.

There are conflicting results regarding the life-extending effect of this group of drugs. On the one hand, the prolongevity effect of serotonin antagonist mianserin was demonstrated in [(Petrascheck M, Ye X, Buck LB: An antidepressant that extends lifespan in adult Caenorhabditis elegans; Nature, Nov. 22, 2007; 450 (7169): 553-6, PMID 18033297)] and in [˜\cite{ye2013pharmacological}], the study we used for validation of our genetic scores of drugs. These findings, however, were not replicated in another study˜\cite{zarse2008antidepressants}, most probably due to the difference in experimental growth conditions (liquid vs solid media). Furthermore, serotonin antagonist, ketanserin, affected lifespan in a diet-dependent manner in experiments with D. melanogaster˜\cite{ro2016serotonin} by altering protein food value perception: it did not affect lifespan in fixed-diet conditions and increased lifespan of flies that were allowed to chose nutrients. It would be harder to expect that such complicated biology may be reproduced in cell-based experiments in LINCS L1000, therefore the appearance of pharmacological targets involved in serotonin and dopamine signalling may be an artifact of our computational procedure. Even if the predictions of our network are real, we believe that the longevity effects of the drugs from this class would be hard to confirm and study in animal models and clinical trials.

The LINCS L1000 dataset provides transcriptional responses of multiple human cells to genetic interventions such as shRNA ($4000$ knockouts out of roughly $20k$ protein-coding genes in the human genome˜\cite{salzberg2018open}). The dimensionality reduction achieved by the deep neural network allowed to build and cross-validate the all-cause mortality risk model in UKB with as little as $˜200k$ exomes available. We, therefore, perceive the results of this study as an encouraging demonstration of modern AM/ML technology to problems involving reverse engineering of molecular interactions in complex biological systems. We expect that the upcoming release of the extended UKB WES database this fall will use our approach to a wide range of phenotypes, especially in relation to major but less prevalent diseases and conditions, than aging.

### Materials and Methods

Deep Learning Gene Expression Representations from LINCS L1000

The DNN was constructed of the three blocks: DenseNet block, Embeddings, and Perturbagen classifier, see Fig. ??. The DenseNet was implemented as suggested in [13] with 32 hidden layers and a growth rate of 48. The Embeddings layer is a dense layer with 2514 Input size and 20 output size. The perturbagen classifier was used to predict the perturbagen class (pert_iname label in the L1000 dataset) from the embedding vector using additive margin Softmax (AM-Softmax) [22] with the margin value was set to 0.2 as in [13].

The training data included profiles from LINCS PHASE I (GEO accession number GSE92742) and PHASE II (GEO accession number GSE70138). We removed samples related to cell lines in which less than 5000 profiles were measured. The preprocessed dataset involving 1467244 gene expression profiles corresponding to 27870 unique compound perturbation classes was split into the training and test datasets at 80/20 ratio. The test dataset did not include gene expression signatures from the training dataset.

Risk Models with Small Effects
MRR
log-likelihood test
cross-validation

### Gene Set Enrichment Analysis

To obtain insights into the biology of human longevity we used LINCS L1000 dataset for search of gene transcripts most associated with the longevity risk scores. We utilized both landmark genes and imputed transcripts at level 4 of LINCS L1000 data, which formed a subset of 12328 genes. Next, the Pearson correlation coefficient was calculated between the each gene expression and longevity score in perturbagens profiles. Genes with the strongest positive and negative correlations were used for the gene-set enrichment analysis, the background was the list of all 12328 genes in L1000.

We used Fisher's exact test for estimating enrichment of gene lists implemented in GSEApy python software package. The analysis was done using ‘KEGG_2019_Human’ and ‘Rare_Diseases_AutoRIF_Gene_Lists’ libraries from Enrichr database. We report Benjamini-Hochberg adjusted p-values and combined scores defined as −log₁₀(p)*OR.

### Target Deconvolution

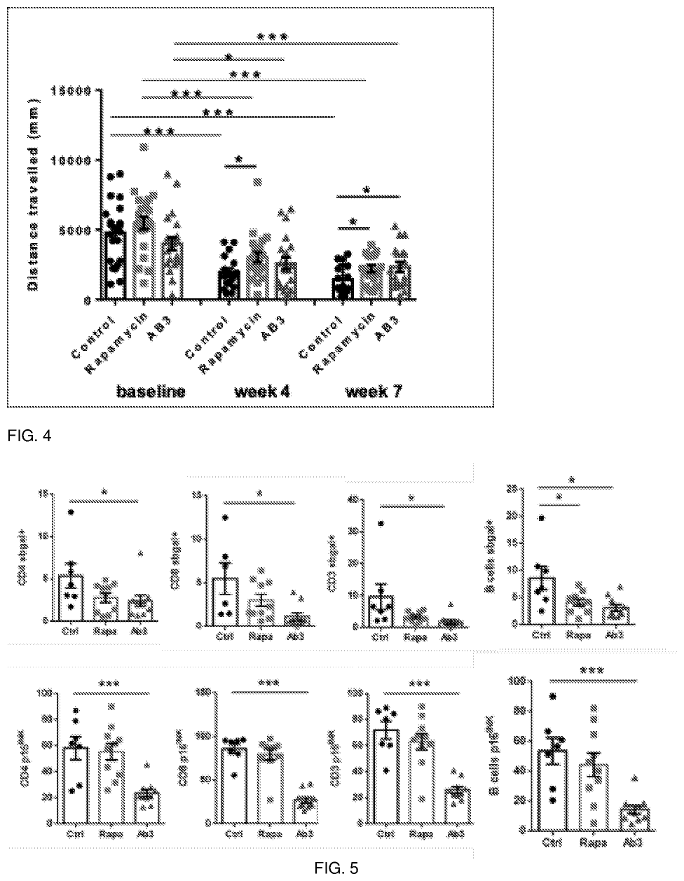

ROC AUC in FIG. 4 is
bootstrapping ROC AUC in
FIG. 5 is shuffling of the label
the method is from [23]

### BIBLIOGRAPHY

[1] J. W. Scannell, A. Blanckley, H. Boldon, and B. Warrington, “Diagnosing the decline in pharmaceutical R&D efficiency,” Nat. Rev. Drug Discov., vol. 11, no. 3, pp. 191-200, 2012.
[2] P. Morgan et al., “Can the flow of medicines be improved? Fundamental pharmacokinetic and pharmacological principles toward improving Phase II survival,” Drug Discov. Today, vol. 17, no. 9, pp. 419-424, 2012.
[3] M. R. Nelson et al., “The support of human genetic evidence for approved drug indications,” Nat. Genet., vol. 47, no. 8, pp. 856-860, 2015.
[4] E. A. King, J. W. Davis, and J. F. Degner, “Are drug targets with genetic support twice as likely to be approved? Revised estimates of the impact of genetic support for drug mechanisms on the probability of drug approval,” bioRxiv, vol. 15, no. 12, p. e1008489, 2019.
[5] A. C. Need and D. B. Goldstein, “Whole genome association studies in complex diseases: where do we stand?,” Dialogues Clin. Neurosci., vol. 12, no. 1, pp. 37-46, 2010.
[6] C. V Van Hout et al., “Whole exome sequencing and characterization of coding variation in 49,960 individuals in the UK Biobank,” bioRxiv, p. 572347, January 2019.
[7] D. G. MacArthur et al., “A systematic survey of loss-of-function variants in human protein-coding genes,” Science (80-.)., vol. 335, no. 6070, pp. 823-828, February 2012.
[8] A. V. Shindyapina, A. A. Zenin, A. E. Tarkhov, D. Santesmasses, P. O. Fedichev, and V. N. Gladyshev, “Germline burden of rare damaging variants negatively affects human healthspan and lifespan,” Elife, vol. 9, April 2020.

### Main Tables

TABLE 1
Target deconvolution of the genetic longevity score using the protein binding information
from ChEMBL (p-values are reported after Benjamini-Hochberg correction)

| gene | | | Number of | Delta | p-value, | |
|---|---|---|---|---|---|---|
| symbol | gene name | gene classification | drugs | score | (KS test) | Cytotoxic |
| MTOR | Serine/threonine-protein kinase | Atypical protein kinase | 15 | 5.046 | 3E−10 | Yes |
| | mTOR | FRAP subfamily | | | | |
| PIK3CB | PI3-kinase p110-beta subunit | Transferase | 12 | 4.186 | 0.0001 | Yes |
| PIK3CD | PI3-kinase p110-delta subunit | Transferase | 15 | 2.133 | 0.0001 | Yes |
| PIK3CG | PI3-kinase p110-gamma subunit | Transferase | 17 | 1.652 | 0.007 | Yes |
| ERBB3 | Receptor tyrosine-protein kinase | Tyrosine protein kinase | 11 | 1.601 | 0.04 | Yes |
| | erbB-3 | EGFR family | | | | |
| ERBB2 | Receptor protein-tyrosine kinase | Tyrosine protein kinase | 17 | 1.593 | 0.008 | Yes |
| | erbB-2 | EGFR family | | | | |
| MAP4K5 | Mitogen-activated protein kinase | STE protein kinase KHS | 11 | 1.4 | 0.01 | Yes |
| | kinase kinase kinase 5 | subfamily | | | | |
| PIK3CA | PI3-kinase p110-alpha subunit | Transferase | 23 | 1.291 | 0.004 | Yes |
| SLC6A2 | Norepinephrine transporter | SLC06 neurotransmitter | 30 | 1.099 | 0.0008 | No |
| | | transporter family | | | | |
| BLK | Tyrosine-protein kinase BLK | Tyrosine protein kinase | 13 | 1.082 | 0.05 | Yes |
| | | Src family | | | | |
| HDAC10 | Histone deacetylase 10 | HDAC class IIb | 11 | 0.937 | 0.0001 | Yes |
| HDAC8 | Histone deacetylase 8 | HDAC class I | 12 | 0.747 | 9E−05 | Yes |
| HDAC7 | Histone deacetylase 7 | HDAC class IIa | 10 | 0.747 | 0.0004 | Yes |
| HTR7 | Serotonin 7 (5-HT7) receptor | Serotonin receptor | 30 | 0.723 | 0.003 | No |
| SLC6A3 | Dopamine transporter | SLC06 neurotransmitter | 26 | 0.63 | 0.002 | No |
| | | transporter family | | | | |
| HDAC6 | Histone deacetylase 6 | HDAC class IIb | 21 | 0.572 | 7E−06 | Yes |
| HRH1 | Histamine H1 receptor | Histamine receptor | 51 | 0.518 | 0.0005 | No |
| EGFR | Epidermal growth factor receptor | Tyrosine protein kinase | 33 | 0.488 | 0.02 | Yes |
| | erbB1 | EGFR family | | | | |
| HDAC2 | Histone deacetylase 2 | HDAC class I | 30 | 0.457 | 1E−06 | Yes |
| HDAC3 | Histone deacetylase 3 | HDAC class I | 28 | 0.456 | 1E−06 | Yes |
| HDAC1 | Histone deacetylase 1 | HDAC class I | 33 | 0.4 | 6E−06 | Yes |
| KCNH2 | HERG | Voltage-gated potassium | 31 | 0.389 | 0.007 | No |
| | | channel | | | | |
| HTR6 | Serotonin 6 (5-HT6) receptor | Serotonin receptor | 40 | 0.377 | 0.005 | No |
| DRD5 | Dopamine D5 receptor | Dopamine receptor | 31 | 0.367 | 0.01 | No |
| HTR2B | Serotonin 2b (5-HT2b) receptor | Serotonin receptor | 84 | 0.243 | 0.001 | No |
| SLC6A4 | Serotonin transporter | SLC06 neurotransmitter | 47 | 0.21 | 0.01 | No |
| | | transporter family | | | | |
| HTR2A | Serotonin 2a (5-HT2a) receptor | Serotonin receptor | 101 | 0.209 | 0.002 | No |
| HTR2C | Serotonin 2c (5-HT2c) receptor | Serotonin receptor | 80 | 0.206 | 0.007 | No |
| DRD1 | Dopamine D1 receptor | Dopamine receptor | 54 | 0.149 | 0.05 | No |
| ADRA1A | Alpha-1a adrenergic receptor | Adrenergic receptor | 86 | 0.146 | 0.007 | No |
| ADRA1B | Alpha-1b adrenergic receptor | Adrenergic receptor | 79 | 0.145 | 0.01 | No |
| DRD3 | Dopamine D3 receptor | Dopamine receptor | 77 | 0.137 | 0.02 | No |
| ADRA1D | Alpha-1d adrenergic receptor | Adrenergic receptor | 77 | 0.134 | 0.01 | No |
| DRD2 | Dopamine D2 receptor | Dopamine receptor | 98 | 0.131 | 0.01 | No |
| CNR1 | Cannabinoid CB1 receptor | Cannabinoid receptor | 18 | 0.122 | 0.05 | No |
| DRD4 | Dopamine D4 receptor | Dopamine receptor | 47 | 0.122 | 0.03 | No |

The following non-limiting examples are illustrative of the present application:

All Examples 1-35 are prophetic

### Example 1

### Animal Treatment/In Vivo Experiments

To evaluate the effect of compound selected from the table 1 ab and Table 1c on frailty and frailty-related phenotypes in C57BL/6 male mice aged 106 weeks, the animals are treated with the compound selected from the table 1 ab and Table 1c or composition, comprising such compound via IV injection. In other example, with the same dosage and regimen as shown in this Example 1 the animals are treated with the compound selected from the table 1 ab, Table 1c and Table 1d, including but not limited to compounds selected from the group consisting of: CYCLOSPORINE, TARIQUIDAR, MARIMASTAT, PRINOMASTAT, APRATASTAT, YOHIMBINE, QUETIAPINE, DOXEPIN, MIANSERIN, ERGOTAMINE, PIPAMAZINE, PHENTOLAMINE, LISURIDE, TAMSULOSIN, DEXMEDETOMIDINE, INDORAMIN, RISPERIDONE, SERTINDOLE, XYLOMETAZOLINE, NAPHAZOLINE, TETRAHYDROZOLINE, DESIPRAMINE, ALFUZOSIN, SILODOSIN, TERAZOSIN, OLANZAPINE, ZOTEPINE, DROPERIDOL, THIORIDAZINE, CHLORPROMAZINE, FLUPHENAZINE, CARVEDILOL, OXYMETAZOLINE, HALOPERIDOL, PHENOXYBENZAMINE, NAFTOPIDIL, DOXAZOSIN, BROMOCRIPTINE, CLOZAPINE, PRAZOSIN, DIHYDROERGOTAMINE, CLOMIPRAMINE, AMITRIPTYLINE, IMIPRAMINE, NORTRIPTYLINE, ASTEMIZOLE, PROMAZINE, ZIPRASIDONE, PHENYLEPHRINE, CISAPRIDE, DAPIPRAZOLE, ILOPERIDONE, MORPHINE, ZOLMITRIPTAN, LOFEXIDINE, FIPAMEZOLE, FLUOXETINE, ATIPAMEZOLE, APRACLONIDINE, LEVONORDEFRIN, IDAZOXAN, BRIMONIDINE, GUANABENZ, EPINEPHRINE, NOREPINEPHRINE, CLONIDINE, CLONIDINE, METERGOLINE, PROCHLORPERAZINE, LY-2811376, LANABECESTAT, AZD-3839, VERUBECESTAT, BIRABRESIB, MIVEBRESIB, VORINOSTAT, ALOBRESIB, AZD-5153, PANOBINOSTAT, ACETAZOLAMIDE, BRINZOLAMIDE, MAFENIDE, INDISULAM, INDAPAMIDE, DTP-348, TOPIRAMATE, LEVOSULPIRIDE, DORZOLAMIDE, SULPIRIDE, CHLORTHALIDONE, METHAZOLAMIDE, NILOTINIB, ETHOXZOLAMIDE, ZONISAMIDE, DICHLORPHENAMIDE, SULTHIAME, METOLAZONE, TRICHLORMETHIAZIDE, SACCHARIN, LUTEOLIN, ISOQUERCETIN, DAIDZEIN, FAMOTIDINE, AZD-5438, TRILACICLIB, RGB-286638, ZOTIRACICLIB, LEROCICLIB, PHA-793887, PALBOCICLIB, HEXAMETHYL PARAROSANILINE, RG-547, DINACICLIB, FORETINIB, AST-487, AT-7519, AZD-7762, UCN-01, SUNITINIB, SILMITASERTIB, LESTAURTINIB, EDICOTINIB, ILORASERTIB, PEXIDARTINIB, TAK-593, PAZOPANIB, TANDUTINIB, DOVITINIB, CEP-32496, QUIZARTINIB, LINIFANIB, MOTESANIB, MASITINIB, DASATINIB, IMATINIB, SU-014813, RELACATIB, BALICATIB, ODANACATIB, ROTIGOTINE, CLEBOPRIDE RACLOPRIDE, SUMANIROLE, PRAMIPEXOLE, PF-00217830, PALIPERIDONE, BENPERIDOL, BREXPIPRAZOLE, BIFEPRUNOX, HALOPERIDOL DECANOATE, ARMODAFINIL, MODAFINIL, MESORIDAZINE, ASENAPINE, BLONANSERIN CLOTHIAPINE, FALLYPRIDE, ROPINIROLE, SARIZOTAN, PERPHENAZINE, DOPAMINE, APOMORPHINE, PIMOZIDE, ARIPIPRAZOLE, RITANSERIN CHLORPROTHIXENE, E-FLUPENTIXOL, TRIFLUOPERAZINE, DOMPERIDONE Adoprazine, AMISULPRIDE, PERGOLIDE, CARIPRAZINE, FLUPENTIXOL DRONABINOL, CLEMASTINE, KETANSERIN, LOXAPINE, ECOPIPAM, RALOXIFENE, TAMOXIFEN, CLOMIPHENE, ENCLOMIPHENE, IFENPRODIL, TRIPARANOL, TESEVATINIB, ROCILETINIB, CP-724714, AEE-788, CUDC-101, FALNIDAMOL, AZD-3759, DOCETAXEL, SAPITINIB, NAQUOTINIB, NAZARTINIB, OLMUTINIB, MAVELERTINIB, PF-06459988, DACOMITINIB, GEFITINIB, ICOTINIB, NERATINIB, TAK-285, MIDOSTAURIN, AFATINIB, VANDETANIB, LAPATINIB, ERLOTINIB, CANERTINIB, IBRUTINIB, POZIOTINIB, VARLITINIB, OSIMERTINIB, PELITINIB, SORAFENIB, BOSUTINIB, PF-04457845, MK-3168, ORLISTAT, URB-597, AT-9283, TAFETINIB, CEP-11981, LUCITANIB, KRN-633, MK-2461, TIVOZANIB, SEMAXANIB, AXITINIB, BRIVANIB, VATALANIB, CEDIRANIB, BRIGATINIB, CEP-5214, BEMCENTINIB, CABOZANTINIB, CRENOLANIB, PACRITINIB, PONATINIB, BARASERTIB, ENMD-2076, CEP-1347, R-406, TOZASERTIB, KW-2449, FEDRATINIB, NINTEDANIB, GILTERITINIB, TELATINIB, LENVATINIB, CHLORAMBUCIL, FIMEPINOSTAT, QUISINOSTAT, ABEXINOSTAT, Givinostat, NANATINOSTAT, PYROXAMIDE, ROMIDEPSIN, MOCETINOSTAT, ENTINOSTAT, TRICHOSTATIN, BELINOSTAT, R-306465, BENDAMUSTINE, RICOLINOSTAT, CITARINOSTAT, AR-42, MAPROTILINE, PYRILAMINE, METHAPYRILENE, AZELASTINE, MIRTAZAPINE, TRIPROLIDINE, GSK-1004723, MIZOLASTINE, RUPATADINE, AZATADINE CYPROHEPTADINE, HYDROXYZINE, CINNARIZINE, DESLORATADINE, CYCLIZINE, EBASTINE, BENZTROPINE, DIMETHINDENE, LEVOCETIRIZINE, CETIRIZINE, MEPAZINE, TERFENADINE, PROMETHAZINE, DEXCHLORPHENIRAMINE CHLORPHENIRAMINE, AMOXAPINE, KETOTIFEN, PU-H71, TANESPIMYCIN LUMINESPIB, GANETESPIB, BIIB021, ALVESPIMYCIN, ALVESPIMYCIN, GELDANAMYCIN, TRAZODONE, PRUVANSERIN, NELOTANSERIN, VOLINANSERIN, TEGASEROD, VELUSETRAG, TEMANOGREL, SEROTONIN, LYSERGIDE METHYSERGIDE, ERGONOVINE, NEFAZODONE, METHYLERGONOVINE, OXITRIPTAN, LORCASERIN, CABERGOLINE, SUMATRIPTAN, CHLOROPHENYLPIPERAZINE, VABICASERIN, IDALOPIRDINE, CERLAPIRDINE, INTEPIRDINE, LANDIPIRDINE, LATREPIRDINE, JNJ-18038683, BMS-754807, CERITINIB, XL-228, LINSITINIB, AS-602868, DOFETILIDE, IBUTILIDE HALOFANTRINE, VERAPAMIL, ANAGLIPTIN, CITALOPRAM, QUINIDINE, ESCITALOPRAM, QUININE, OSI-632, OSI-930, RIVOCERANIB, RG-1530, AG-13958, SITRAVATINIB, ALTIRATINIB, REGORAFENIB, CRIZOTINIB, SIROLIMUS, SARACATINIB, BAFETINIB, TALMAPIMOD, NEFLAMAPIMOD, QUERCETIN, CI-1040, DORAMAPIMOD, ARRY-797, PH-797804, VX-702, LOSMAPIMOD, TAK-715, PAMAPIMOD, R-1487, JNJ-49095397, CTS-1027, ZOTAROLIMUS, BGT-226, RG-7603, TACROLIMUS, EVEROLIMUS, PKI-179, AZD-8055, DACTOLISIB, GEDATOLISIB, OMIPALISIB, VISTUSERTIB, PF-04691502, APITOLISIB, SAPANISERTIB, BORTEZOMIB, IXAZOMIB, PD-0166285, RAF-265, TG100-115, TASELISIB, PICTILISIB, BUPARLISIB, COPANLISIB, ALPELISIB, BIMIRALISIB, AZD-8835, AZD-6482, GSK-2636771, NEMIRALISIB, GS-9901, LENIOLISIB, IDELALISIB, DUVELISIB, IPI-549, TAK-960, ONVANSERTIB, RIGOSERTIB, VOLASERTIB, GSK-461364, BI-2536, ADAVOSERTIB, LINOLEIC ACID, PEMAFIBRATE, NAMODENOSON, GW501516, INT131, EFATUTAZONE, MK-0533, ROSIGLITAZONE, MURAGLITAZAR, FARGLITAZAR, SEMAGACESTAT, BEGACESTAT, AVAGACESTAT, NIROGACESTAT, LIAFENSINE, NOMIFENSINE, 1-(3,4-DICHLOROPHENYL)-6-(METHOXYMETHYL)-3-AZABICYCLO[4.1.0]HEPTANE (ENANTIOMERIC MIX), ESREBOXETINE, REBOXETINE, DULOXETINE, ATOMOXETINE, MAZINDOL, AMPHETAMINE, DEXTROAMPHETAMINE, MILNACIPRAN, LEVOMILNACIPRAN, PROTRIPTYLINE, IOFLUPANE, FLUVOXAMINE, PYROVALERONE, VANOXERINE, PAROXETINE, LITOXETINE, AMITIFADINE, UK-390957, INDALPINE, VORTIOXETINE, VILAZODONE, MIDOMAFETAMINE, PRIMAQUINE, COCAINE, DEXTROMETHORPHAN, SERTRALINE, VENLAFAXINE, OZAGREL, TERBOGREL, DOLASTATIN-10, PATUPILONE via Intraperitoneal (IP) route of administration. In yet another example, with the same dosage and regimen as shown in this Example 1 the animals are treated subcutaneously. In yet another example, with the same dosage and regimen as shown in this Example 1 the animals are treated by oral gavage. In yet another example, with the same dosage and regimen as shown in this Example 1 the animals are treated orally with the food mixed with the compound selected from the table 1 ab and Table 1c.

Any one of the compounds selected from Tables 1 ab and Table 1c are dissolved in at least one of the following ways:
1) dissolved in DMSO to 10 mM stocks and used in the final assay buffer with minimum 1% DMSO (for 10 micro M final measurements) and with 2% DMSO (for 200 micro M final measurements),
2) dissolved in water to 200 micro M stock solution. The stock solution is then used for further dilutions with assay buffer, and
3) any other dissolution known in the field.

The compound selected from the table 1 ab and Table 1c in any other example is dissolved by any one of the methods described if better method is not suggested here or by the expert.

In one example, the compound, selected from the table 1 ab and Table 1c or composition, comprising such compound is administered in a dosage 20 times less than its LD50.

In another example, the compound, selected from the table 1 ab and Table 1c or composition, comprising such compound is administered in a Maximum Tolerated dose.

In yet another example, the compound selected from the table 1 ab and Table 1c is administered by intraperitoneal injection (IP) in a doses: group #1—1 mg/kg, group #2—10 mg/kg, group #3—100 mg/kg.

In yet another example, the compound selected from the table 1 ab and Table 1c or composition, comprising such compound is administered in a dosage selected from the group consisting of: 10 times less than its LD50, 50 times less than its LD50, 100 times less than its LD50, 500 times less than its LD50, 1000 times less than its LD50, 5000 times less than its LD50, 10000 times less than its LD50, Maximum Tolerated dose, or in the dosage in which such compound was found effective in mice for at least one other indication known in the art.

The compound selected from the table 1 ab and Table 1c or composition, comprising such compound is administered in the one of the following regimens: once, once a day for 1 week, once every two days for 1 week, once every two days for 2 weeks, once every week for 1 month, once every two days for 1 month, once every two days or once every week until at least one symptom of aging is alleviated, in the regimen in which this compound was effective in animal model for its initial indication, in any other regimen described in this application, in any other regimen reasonably defined by the expert in the field taking into the account the knowledge about the compound. In some examples, compound selected from the table 1 ab and Table 1c is compound selected from the group consisting of: CYCLOSPORINE, TARIQUIDAR, MARIMASTAT, PRINOMASTAT, APRATASTAT, YOHIMBINE, QUETIAPINE, DOXEPIN, MIANSERIN, ERGOTAMINE, PIPAMAZINE, PHENTOLAMINE, LISURIDE, TAMSULOSIN, DEXMEDETOMIDINE, INDORAMIN, RISPERIDONE, SERTINDOLE, XYLOMETAZOLINE, NAPHAZOLINE, TETRAHYDROZOLINE, DESIPRAMINE, ALFUZOSIN, SILODOSIN, TERAZOSIN, OLANZAPINE, ZOTEPINE, DROPERIDOL, THIORIDAZINE, CHLORPROMAZINE, FLUPHENAZINE, CARVEDILOL, OXYMETAZOLINE, HALOPERIDOL, PHENOXYBENZAMINE, NAFTOPIDIL, DOXAZOSIN, BROMOCRIPTINE, CLOZAPINE, PRAZOSIN, DIHYDROERGOTAMINE, CLOMIPRAMINE, AMITRIPTYLINE, IMIPRAMINE, NORTRIPTYLINE, ASTEMIZOLE, PROMAZINE, ZIPRASIDONE, PHENYLEPHRINE, CISAPRIDE, DAPIPRAZOLE, ILOPERIDONE, MORPHINE, ZOLMITRIPTAN, LOFEXIDINE, FIPAMEZOLE, FLUOXETINE, ATIPAMEZOLE, APRACLONIDINE, LEVONORDEFRIN, IDAZOXAN, BRIMONIDINE, GUANABENZ, EPINEPHRINE, NOREPINEPHRINE, CLONIDINE, CLONIDINE, METERGOLINE, PROCHLORPERAZINE, LY-2811376, LANABECESTAT, AZD-3839, VERUBECESTAT, BIRABRESIB, MIVEBRESIB, VORINOSTAT, ALOBRESIB, AZD-5153 PANOBINOSTAT, ACETAZOLAMIDE, BRINZOLAMIDE, MAFENIDE, INDISULAM INDAPAMIDE, DTP-348, TOPIRAMATE, LEVOSULPIRIDE, DORZOLAMIDE, SULPIRIDE, CHLORTHALIDONE, METHAZOLAMIDE, NILOTINIB, ETHOXZOLAMIDE, ZONISAMIDE, DICHLORPHENAMIDE, SULTHIAME, METOLAZONE, TRICHLORMETHIAZIDE, SACCHARIN, LUTEOLIN, ISOQUERCETIN, DAIDZEIN, FAMOTIDINE, AZD-5438, TRILACICLIB, RGB-286638, ZOTIRACICLIB, LEROCICLIB, PHA-793887, PALBOCICLIB, HEXAMETHYL PARAROSANILINE, RG-547, DINACICLIB, FORETINIB, AST-487, AT-7519, AZD-7762, UCN-01, SUNITINIB, SILMITASERTIB, LESTAURTINIB, EDICOTINIB, ILORASERTIB, PEXIDARTINIB, TAK-593, PAZOPANIB, TANDUTINIB, DOVITINIB, CEP-32496, QUIZARTINIB, LINIFANIB, MOTESANIB, MASITINIB, DASATINIB, IMATINIB, SU-014813, RELACATIB, BALICATIB, ODANACATIB, ROTIGOTINE, CLEBOPRIDE, RACLOPRIDE, SUMANIROLE, PRAMIPEXOLE, PF-00217830, PALIPERIDONE, BENPERIDOL, BREXPIPRAZOLE, BIFEPRUNOX, HALOPERIDOL DECANOATE, ARMODAFINIL, MODAFINIL, MESORIDAZINE, ASENAPINE, BLONANSERIN, CLOTHIAPINE, FALLYPRIDE, ROPINIROLE, SARIZOTAN, PERPHENAZINE, DOPAMINE, APOMORPHINE, PIMOZIDE, ARIPIPRAZOLE, RITANSERIN, CHLORPROTHIXENE, E-FLUPENTIXOL, TRIFLUOPERAZINE, DOMPERIDONE, Adoprazine, AMISULPRIDE, PERGOLIDE, CARIPRAZINE, FLUPENTIXOL, DRONABINOL, CLEMASTINE, KETANSERIN, LOXAPINE, ECOPIPAM, RALOXIFENE, TAMOXIFEN, CLOMIPHENE, ENCLOMIPHENE, IFENPRODIL, TRIPARANOL, TESEVATINIB, ROCILETINIB, CP-724714, AEE-788, CUDC-101, FALNIDAMOL, AZD-3759, DOCETAXEL, SAPITINIB, NAQUOTINIB, NAZARTINIB, OLMUTINIB, MAVELERTINIB, PF-06459988, DACOMITINIB, GEFITINIB, ICOTINIB, NERATINIB, TAK-285, MIDOSTAURIN, AFATINIB, VANDETANIB, LAPATINIB, ERLOTINIB, CANERTINIB, IBRUTINIB, POZIOTINIB, VARLITINIB, OSIMERTINIB, PELITINIB, SORAFENIB, BOSUTINIB, PF-04457845, MK-3168, ORLISTAT, URB-597, AT-9283, TAFETINIB, CEP-11981, LUCITANIB, KRN-633, MK-2461, TIVOZANIB, SEMAXANIB, AXITINIB, BRIVANIB, VATALANIB, CEDIRANIB, BRIGATINIB, CEP-5214, BEMCENTINIB, CABOZANTINIB, CRENOLANIB, PACRITINIB, PONATINIB, BARASERTIB, ENMD-2076, CEP-1347, R-406, TOZASERTIB, KW-2449, FEDRATINIB, NINTEDANIB, GILTERITINIB, TELATINIB, LENVATINIB, CHLORAMBUCIL, FIMEPINOSTAT, QUISINOSTAT, ABEXINOSTAT, Givinostat, NANATINOSTAT, PYROXAMIDE, ROMIDEPSIN, MOCETINOSTAT, ENTINOSTAT, TRICHOSTATIN, BELINOSTAT, R-306465, BENDAMUSTINE, RICOLINOSTAT, CITARINOSTAT, AR-42, MAPROTILINE, PYRILAMINE, METHAPYRILENE, AZELASTINE, MIRTAZAPINE, TRIPROLIDINE, GSK-1004723 MIZOLASTINE, RUPATADINE, AZATADINE, CYPROHEPTADINE, HYDROXYZINE, CINNARIZINE, DESLORATADINE, CYCLIZINE, EBASTINE, BENZTROPINE, DIMETHINDENE, LEVOCETIRIZINE, CETIRIZINE, MEPAZINE, TERFENADINE, PROMETHAZINE, DEXCHLORPHENIRAMINE, CHLORPHENIRAMINE, AMOXAPINE, KETOTIFEN, PU-H71, TANESPIMYCIN, LUMINESPIB, GANETESPIB, BIIB021, ALVESPIMYCIN, ALVESPIMYCIN, GELDANAMYCIN, TRAZODONE, PRUVANSERIN, NELOTANSERIN, VOLINANSERIN, TEGASEROD, VELUSETRAG, TEMANOGREL, SEROTONIN, LYSERGIDE, METHYSERGIDE, ERGONOVINE, NEFAZODONE, METHYLERGONOVINE, OXITRIPTAN, LORCASERIN, CABERGOLINE, SUMATRIPTAN, CHLOROPHENYLPIPERAZINE, VABICASERIN, IDALOPIRDINE, CERLAPIRDINE, INTEPIRDINE, LANDIPIRDINE, LATREPIRDINE, JNJ-18038683, BMS-754807, CERITINIB, XL-228, LINSITINIB, AS-602868, DOFETILIDE, IBUTILIDE, HALOFANTRINE, VERAPAMIL, ANAGLIPTIN, CITALOPRAM, QUINIDINE, ESCITALOPRAM, QUININE, OSI-632, OSI-930, RIVOCERANIB, RG-1530, AG-13958, SITRAVATINIB, ALTIRATINIB, REGORAFENIB, CRIZOTINIB, SIROLIMUS, SARACATINIB, BAFETINIB, TALMAPIMOD, NEFLAMAPIMOD, QUERCETIN, CI-1040, DORAMAPIMOD, ARRY-797, PH-797804, VX-702, LOSMAPIMOD, TAK-715, PAMAPIMOD, R-1487, JNJ-49095397, CTS-1027, ZOTAROLIMUS, BGT-226, RG-7603, TACROLIMUS, EVEROLIMUS, PKI-179, AZD-8055, DACTOLISIB, GEDATOLISIB, OMIPALISIB, VISTUSERTIB, PF-04691502, APITOLISIB, SAPANISERTIB, BORTEZOMIB, IXAZOMIB, PD-0166285, RAF-265, TG100-115, TASELISIB, PICTILISIB, BUPARLISIB, COPANLISIB, ALPELISIB, BIMIRALISIB, AZD-8835, AZD-6482, GSK-2636771, NEMIRALISIB, GS-9901, LENIOLISIB, IDELALISIB, DUVELISIB, IPI-549, TAK-960, ONVANSERTIB, RIGOSERTIB, VOLASERTIB, GSK-461364, BI-2536, ADAVOSERTIB, LINOLEIC ACID, PEMAFIBRATE, NAMODENOSON, GW501516, INT131, EFATUTAZONE, MK-0533, ROSIGLITAZONE, MURAGLITAZAR, FARGLITAZAR, SEMAGACESTAT, BEGACESTAT, AVAGACESTAT, NIROGACESTAT, LIAFENSINE, NOMIFENSINE, 1-(3,4-DICHLOROPHENYL)-6-(METHOXYMETHYL)-3-AZABICYCLO[4.1.0]HEPTANE (ENANTIOMERIC MIX), ESREBOXETINE, REBOXETINE, DULOXETINE, ATOMOXETINE, MAZINDOL, AMPHETAMINE, DEXTROAMPHETAMINE, MILNACIPRAN LEVOMILNACIPRAN, PROTRIPTYLINE, IOFLUPANE, FLUVOXAMINE, PYROVALERONE, VANOXERINE, PAROXETINE, LITOXETINE, AMITIFADINE, UK-390957, INDALPINE, VORTIOXETINE, VILAZODONE, MIDOMAFETAMINE, PRIMAQUINE, COCAINE DEXTROMETHORPHAN, SERTRALINE, VENLAFAXINE, OZAGREL, TERBOGREL DOLASTATIN-10, PATUPILONE.

In yet another example, to get the anti-aging effect compound selected from the table 1 ab and Table 1c is administered by the root of administration, in dosage and regimen which were previously used (known in the art) to show the efficacy in mice (or if mice model had not been done in any other animal model, but with the correction in the dosage for the mice) of the same compound in at least one disease or condition. In yet another examples, the dosage is taken two-fold greater than the dosage in which the compound was effective for other disease in mice, or in yet another example the dosage is taken two-fold less. In yet another examples, the dosage is taken ten-fold greater than the dosage in which the compound was effective for other disease in mice, or in other example the dosage is taken ten-fold less than the dosage in which the compound was effective for other disease in mice.

The mice from reference group are treated with 42 ppm of a well-characterized life-extending drug-rapamycin as the positive control. The life-extending properties of rapamycin have been confirmed in multiple experiments, including a large study within the NIH (NIA) Intervention Testing Program (https://www.nia.nih.gov/research/dab/interventions-testing-program-itp).

The interventions in the animals can reduce their age related morbidity and mortality. The effects of the interventions in the animals can be evaluated with the help of the Frailty Index and Open Field Test. Markers of senescence in peripheral lymphocytes, synaptoplasticity, and, ultimately, survival are also measured.

### Survival

The prophetic survival curves from the experiment are shown in FIG. 2. Animals in the one of the Treatment groups (treated with the compounds selected from the table 1 ab and Table 1c or compositions, comprising such compound) and in the rapamycin treatment groups demonstrate a significant improvement in lifespan (see FIG. 2).

### Frailty Index

The Frailty Index (FI) is measured as in Whitehead et al (2014) and consists of 31 phenotypes that are indicators of age-associated health deterioration including alopecia, physical/musculoskeletal, auditory, ocular, nasal, signatures of digestive and other disorders. Each phenotype is scored on a 0, 0.5 or 1 scale, based on its severity. The 31 metrics share many characteristics of the human frailty indicators and were previously reported to progress similarly with aging in mice and humans (Kane, A. E., et al, 2016). As shown in FIG. 3, treatment with the compound selected from the table 1 ab and Table 1c or compositions, comprising such compound as well as rapamycin demonstrate a significant reduction of Frailty Index.

### Open Field Test

Locomotor activity recordings are carried out using a square open field. Locomotor activity data including total distance traveled (mm) and velocity (mm/s) are measured. Animal motor function deteriorated significantly while ageing. At 4 weeks and 7 weeks after the start of rapamycin treatment (baseline), mice show significant improvement in motor function compared to the control group. At 5 weeks after treatment with the compound selected from the table 1 ab and Table 1c or compositions, comprising such compound (7 weeks after baseline measurement), mice show significant improvement in motor function compared to the control group.

### Senescence

In a study using human peripheral lymphocytes (Liu Y et al, 2009), it was shown that the expression of p16INK4a correlated with donor chronologic age, smoking status, physical inactivity, and plasma interleukin-6 concentration, a marker of human frailty. These data suggest that p16INK4a expression in peripheral lymphocytes may be used as a blood biomarker of frailty. Expression of p16 and beta-galactosidase in peripheral lymphocytes were combined in a single principal component. FIG. 5 shows that animals from the Treatment group show a significantly lower number of senescent cells in all lymphocyte subpopulations compared to control animals.

### Synaptoplasticity

Progressive reduction of structural and functional plasticity is associated with the gradual decline in cognitive function. Moreover, chemotherapy-induced cognitive decline is associated with loss of synaptic integrity and it was demonstrated that cisplatin-treated mice show reduced levels of PSD95 and synaptophysin (Chiang A C A et al., 2019). Synaptophysin immunoreactivity has been identified as a useful marker for presynaptic density, whereas PSD95 is a marker of post-synaptic density. In order to analyze synaptic plasticity in the experiment, Western blot analysis of mouse brain tissue are performed using antibodies against PSD95 and synaptophysin. As shown in FIG. 6, the expression of PSD95 and synaptophysin are significantly higher in the Treatment group compared to the control group.

### Senescence and COVID-19 and Other Respiratory Diseases.

Mortality from acute respiratory diseases, including pneumonia, influenza, and COVID-19 is strongly age-dependent. According to Morbidity and Mortality Weekly Report (MMWR) on the Mar. 18, 2020: “This first preliminary description of outcomes among patients with COVID-19 in the United States indicates that fatality was highest in persons aged ≥85, ranging from 10% to 27%, followed by 3% to 11% among persons aged 65-84 years, 1% to 3% among persons aged 55-64 years, <1% among persons aged 20-54 years, and no fatalities among persons aged ≤19 years”. This is at least partially due to increased frailty associated with aging, which involves immunosenescence (decreased immune function in the elderly), general loss of function, and existing comorbidities. It is known that immunosenescence is a major factor affecting vaccination response, as well as the severity and lethality of infectious diseases. The interventions that reduce the number of senescent cells and alleviate frailty might reduce the risk of severe outcomes due to respiratory infections, including the risk of mortality.

### Example 2

### Effect of Removal of Senescent Cells in Animal Models of Atherosclerosis

The study is to assess the extent to which clearance of senescent cells from plaques in LDLR″″ mice with compound selected from the table 1 ab and Table 1c reduces plaque load. Two groups of LDLR″″ mice (10 weeks) are fed a high fat diet (HFD) (Harlan Teklad TD.88137) having 42% calories from fat, beginning at Week 0 and throughout the study. Two groups (n=50 in each group) of LDLR″″ mice (10 weeks) are fed normal chow (−HFD). From weeks 0-2, one group of HFD mice and −HFD mice are treated with compound selected from the table 1 ab and Table 1c with the dosage and root of administration selected from the Example 1.

In one example, the compound, selected from the table 1 ab and Table 1c or composition, comprising such compound is administered in a dosage 20 times less than its LD50 by intraperitoneal injection.

In another example, the compound, selected from the table 1 ab and Table 1c or composition, comprising such compound is administered in a Maximum Tolerated dose by intraperitoneal injection.

In yet another example, the compound selected from the table 1 ab and Table 1c is administered by intraperitoneal injection (IP) in a doses: group #1—1 mg/kg, group #2—10 mg/kg, group #3—100 mg/kg.

In yet another example, the treatment by the compound selected from the table 1 ab and Table 1c consists in single administration of the compound selected from the table 1 ab and Table 1c in the dosage selected from this example.

In yet another example, dosage, regimen and root of administration are selected from the Example 1.

In yet another example, one treatment cycle is 14 days treatment, 14 days off. Vehicle is administered to one group of HFD mice and one group of −HFD mice. At week 4 (timepoint 1), one group of mice are sacrificed and to assess presence of senescent cells in the plaques. For the some of the remaining mice, the compound selected from the table 1 ab and Table 1c and vehicle administration is repeated from weeks 4-6. At week 8 (timepoint 2), the mice are sacrificed and to assess presence of senescent cells in the plaques. The remaining mice are treated with the compound selected from the table 1 ab and Table 1c or vehicle from weeks 8-10. At week 12 (timepoint 3), the mice are sacrificed and to assess the level of plaque and the number of senescent cells in the plaques.

Plasma lipid levels are measured in LDLR″^{/}″ mice fed a HFD and treated with the compound selected from the table 1 ab and Table 1c or vehicle at timepoint 1 as compared with mice fed a −HFD (n=30 per group). Plasma are collected mid-afternoon and analyzed for circulating lipids and lipoproteins. At the end of timepoint 1, LDLR″″ mice fed a HFD and treated with the compound selected from the table 1 ab and Table 1c or vehicle are sacrificed (n=30, all groups), and the aortic arches are dissected for RT-PCR analysis of SASP factors and senescent cell markers. Values are normalized to GAPDH and expressed as fold-change versus age-matched, vehicle-treated LDLR″^{/}″ mice on a normal diet. The data show that clearance of senescent cells with the compound selected from the table 1 ab and Table 1c in LDLR″^{/}″ mice fed a HFD reduce expression of several SASP factors and senescent cell markers, MMP3, MMP13, PAIL, p21, IGFBP2, IL-1A, and IL-1B after 1 treatment cycle.

At the end of timepoint 2, LDLR″^{/}″ mice fed a HFD and treated with the compound selected from the table 1 ab and Table 1c or vehicle (n=30 for all groups) are sacrificed, and aortic arches are dissected for RT-PCR analysis of SASP factors and senescent cell markers. Values are normalized to GAPDH and expressed as fold-change versus age-matched, vehicle-treated LDLR″^{/}″ mice on a normal diet. The data show expression of some SASP factors and senescent cell markers in the aortic arch within HFD mice. Clearance of senescent cells with multiple treatment cycles of the compound selected from the table 1 ab and Table 1c in LDLR″^{/}″ mice fed a HFD reduces expression at least one of the markers.

At the end of timepoint 3, LDLR″^{/}″ mice fed a HED and treated with the compound selected from the table 1 ab and Table 1c A or vehicle (n=30 for all groups) are sacrificed, and aortas are dissected and stained with Sudan IV to detect the presence of lipid. Body composition of the mice are analyzed by MRI, and circulating blood cells are counted by Hemavet. The data show that treatment with the compound selected from the table 1 ab and Table 1c reduces plaques in the descending aorta by −45% (The platelet and lymphocyte counts are equivalent between the compound selected from the table 1 ab and Table 1c and vehicle treated mice. The treatment with the compound selected from the table 1 ab and Table 1c also decreases mass and body fat composition in mice fed a HFD.

### Study

The study assesses the extent to which the compound selected from the table 1 ab and Table 1c based clearance of senescent cells from LDLR″^{/}″/3MR double transgenic mice improves pre-existing atherogenic disease. LDLR″ 73MR double transgenic mice (10 weeks) and LDLR″^{/}″ single transgenic mice (10 weeks) are fed a high fat diet beginning at Week 0 until Week. 12.

In one example, The compound selected from the table 1 ab and Table 1c is administered to groups of mice (dosages for respective groups: 0.1 mg/kg—for group #1, 1 mg/kg—for group #2, 10 mg/kg—for group #3, 100 mg/kg—for group #4 and 1 g/kg—for group #5) intraperitoneally from weeks 12-13 and weeks 14-15.

In another example, the compound, selected from the table 1 ab and Table 1c or composition, comprising such compound is administered in a dosage 20 times less than its LD50 by intraperitoneal injection.

In another example, the compound, selected from the table 1 ab and Table 1c or composition, comprising such compound is administered in a Maximum Tolerated dose by intraperitoneal injection.

In yet another example, the compound selected from the table 1 ab and Table 1c is administered by intraperitoneal injection (IP) in a doses: group #1-1 mg/kg, group #2-10 mg/kg, group #3-100 mg/kg.

In yet another example, the treatment by the compound selected from the table 1 ab and Table 1c consists in single administration of the compound selected from the table 1 ab and Table 1c in the dosage selected from this example.

In yet another example, dosage, regimen and root of administration are selected from the Example 1.

At week 16, the level of plaque and the number of senescent cells in the plaques are determined. The clearance of senescent cells with the compound selected from the table 1 ab and Table 1c in LDLR″ 73MR double transgenic mice fed a HFD (n=30) reduces the % of the aorta covered with plaque as compared to LDLR″^{/}″ mice/HFD controls (n=30). The clearance of senescent cells with the compound selected from the table 1 ab and Table 1c also reduces the plaque cross-sectional area in LDLR″ 73MR double transgenic mice fed a HFD (n=9) as compared to LDLR″^{/}″ mice/HFD controls (n=15).

### Example 3

An Animal Study for Evaluating of the Senolytic Effect of Compound Selected from the Table 1 ab and Table 1c in Mice

The senolytic effect of the compound selected from the Table 1 ab and Table 1c, can be assessed in animal models of senescence. An example of such an animal study is described here. Senescence in animals can be induced through the administration of doxorubicin followed by treatment of the compound selected from the Table 1 ab and Table 1c.

On day 35, mice are sacrificed, and fat and skin are collected for RNA analysis, while lungs are collected and flash frozen for immunomicroscopy analysis. RNA is analyzed for expression of SASP factors (mmp3, IL-6) and senescence markers (p21, i 6, and p53). Frozen lung tissue is analyzed for DNA damage marker (YH2AX).

The mice to be tested contain a transgene insertion of p16-3MR. 3MR (tri-modality reporter) is a fusion protein containing functional domains of a synthetic Renilla luciferase (LUC), monomeric red fluorescence protein (mRFP), and truncated herpes simplex virus (HSV)-1 thymidine kinase ((TK), which allows killing by ganciclovir (GCV). The 3 MR cDNA is inserted in frame with p16 in exon 2, creating a fusion protein containing the first 62 amino acids of pi 6, but does not include the full-length wild-type p16 protein. Insertion of the 3MR cDNA also introduces a stop codon in the p 19^{ARF} reading frame in exon 2.

The effect of the compound selected from the Table 1 ab and Table 1c is analyzed by the reduction of luminescence intensity.

Female C57/B16 p16-3MR mice are treated with Doxorubicin.

Luminescence is measured 10 days later and used as baseline for each mouse (100% intensity).

In one example, The compound selected from the table 1 ab and Table 1c is administered to groups of mice (dosages for respective groups: 0.1 mg/kg—for group #1, 1 mg/kg—for group #2, 10 mg/kg—for group #3, 100 mg/kg—for group #4 and 1 g/kg—for group #5) intraperitoneally daily from day 10 to day 24 post-doxorubicin treatment.

In another example, the compound selected from the Table 1 ab and Table 1c is administered according to the protocol selected from the Example 1.

In another example, the compound, selected from the table 1 ab and Table 1c or composition, comprising such compound is administered in a dosage 20 times less than its LD50 by intraperitoneal injection daily from day 10 to day 24 post-doxorubicin treatment.

In another example, the compound, selected from the table 1 ab and Table 1c or composition, comprising such compound is administered in a Maximum Tolerated dose by intraperitoneal injection daily from day 10 to day 24 post-doxorubicin treatment.

In yet another example, the compound selected from the table 1 ab and Table 1c is administered by intraperitoneal injection (IP) in a doses: group #1—1 mg/kg, group #2—10 mg/kg, group #3—100 mg/kg.

In yet another example, the treatment by the compound selected from the table 1 ab and Table 1c consists in single administration of the compound selected from the table 1 ab and Table 1c in the dosage selected from this example.

In yet another example, dosage, regimen and root of administration are selected from the Example 1.

Luminescence is then measured at day 7, 14, 21, 28, 35 post-compound selected from the Table 1 ab and Table 1c treatments, and final values calculated as % of the baseline values. Control animals (DOXO) are injected with equal volume of PBS.

The level of mRNA of endogenous mmp-3, IL-6, p21, p16, and p53 in the skin and fat from animals after treatment with doxorubicin alone (DOXO) or doxorubicin plus compound selected from the Table 1 ab and Table 1c is plotted. The values represent the fold induction of the particular mRNA compared with untreated control animals.

Immunofluorescence microscopy of lung sections from doxorubicin treated animals (DOXO) and doxorubicin and compound selected from the Table 1 ab and Table 1c can be detected by binding to a primary rabbit polyclonal antibody specific for γH2AX followed by incubation with a secondary goat anti-rabbit antibody, and then counterstained with DAPI. The percent positive cells from immunofluorescence microscopy are calculated and can be represented as percentage of the total number of cells. Data can be obtained from doxorubicin-treated mice (Doxo), and doxorubicin+compound selected from the Table 1 ab and Table 1c-treated mice).

The compound selected from the Table 1 ab and Table 1c can be analyzed for reduced senescence-associated (SA) B-galactosidase (B-gal) intensity of fat biopsies from animals first treated with doxorubicin. Female C57/BL6 p16-3MR mice are treated with doxorubicin. A portion of the doxorubicin treated animals receive compound selected from the Table 1 ab and Table 1c or PBS (DOXO) daily from day 10 to day 24 post-doxorubicin treatment. Three weeks after the compound selected from the Table 1 ab and Table 1c treatment, mice are sacrificed and fat biopsies immediately fixed and stained with a solution containing X-Gal. Untreated animals are used as negative control (CTRL).

### Example 4

Improving at least one of the symptoms or biomarkers of aging or age related disease or condition in mice.

The anti-aging properties of the compounds selected from the table 1 ab and Table 1c or compositions, comprising such compound are confirmed with the following tests:

In one example, The compound selected from the table 1 ab and Table 1c is administered to groups of mice (dosages for respective groups: 0.1 mg/kg—for group #1, 1 mg/kg—for group #2, 10 mg/kg—for group #3, 100 mg/kg—for group #4 and 1 g/kg—for group #5) intraperitoneally from weeks 12-13 and weeks 14-15.

In another example, the compound, selected from the table 1 ab and Table 1c or composition, comprising such compound is administered in a dosage 20 times less than its LD50 by intraperitoneal injection.

In another example, the compound, selected from the table 1 ab and Table 1c or composition, comprising such compound is administered in a Maximum Tolerated dose by intraperitoneal injection.

In yet another example, the compound selected from the table 1 ab and Table 1c is administered by intraperitoneal injection (IP) in a doses: group #1—1 mg/kg, group #2—10 mg/kg, group #3—100 mg/kg.

In one example, the compound selected from the Table 1 ab and Table 1c is administered in dosage, regimen and root of administration as described in Example 1.

Administration of the compound selected from the table 1 ab and Table 1c or composition, comprising such compound produces at least one anti-aging effect, selected from the variety known in the art, including but not limited to those described in this application.

Administration of the compound selected from the table 1 ab and Table 1c or composition, comprising such compound produces at least one of those effects described in Example 1 (that can be less but still statistically meaningful).

Administration of the compound selected from the table 1 ab and Table 1c or composition, comprising such compound produces at least one of those effects described in Example 1 that is at least at the range of shown in Example 1 for treatment group.

Any of the compound selected from Tables 1 ab and Table 1c are dissolved in at least one of the following ways:
1) dissolved in DMSO to 10 mM stocks and used in the final assay buffer with minimum 1% DMSO (for 10 micro M final measurements) and with 2% DMSO (for 200 micro M final measurements),
2) dissolved in DMSO to 10 mM stocks and used in the final assay buffer with minimum 0.1% DMSO
3) dissolved in water to 200 micro M stock solution. The stock solution is then used for further dilutions with assay buffer, and
4) any other dissolution known in the field.

In yet another example, the compound selected from the table 1 ab and Table 1c or composition, comprising such compound is optionally administered in at least one of the dosages listed above once, one time a week during one month, one time a week during two months, one time a week during 3 months, daily during 1 week, 1 month, 6 months or 12 months.

In yet another example, the compound selected from the table 1 ab and Table 1c or composition, comprising such compound is optionally administered once a week until biological age or frailty index of the subject is decreased or the treatment is repeated when the biological age or frailty index of the subject returns to the level when the first treatment had been administered or at least one symptom of aging or aging related disease or condition is alleviated.

The biological age or frailty index can be measured by methods known in the art, including

but not limited to those described in “Identification of a blood test-based biomarker of aging through deep learning of aging trajectories in large phenotypic datasets of mice” (Avchaciov et al., 2020).

### Example 5

### Injection Formulation

A composition comprising an Exemplary Injection Formulation containing a compound selected from the table 1 ab and Table 1c or composition, comprising such compound is prepared. The vial contains 5 mg of any one of the compounds selected from the table 1 ab and Table 1c or composition, comprising such compound as a powder for injection. The powder for injection is reconstituted with sterile water for injections and further diluted in 0.9% sodium chloride solution for infusion. After reconstitution, each vial contains substance for injection. Inactive ingredients include sodium phosphate monobasic monohydrate, sodium phosphate dibasic dihydrate, sucrose and polysorbate 80. In other example, there are no inactive ingredients, and in yet another example inactive ingredients are chosen by the expert in the field.

In other injection formulation examples, compound's selected from the table 1 ab and Table 1c concentration is selected from the group consisting of: 0.01 mg/mL, 0.05 mg/mL, 0.1 mg/mL, 0.5 mg/mL, 1 mg/mL, 5 mg/mL, 10 mg/mL, 50 mg/mL or 100 mg/mL diluted in sodium chloride solution for infusion.

In yet other injection formulation examples, compound's selected from the table 1 ab and Table 1c concentration is selected from the group consisting of: 0.01 mg/mL, 0.05 mg/mL, 0.1 mg/mL, 0.5 mg/mL, 1 mg/mL, 5 mg/mL, 10 mg/mL, 50 mg/mL or 100 mg/mL diluted in sterile water for injections.

In some injection formulation examples such formulations are for IV injections.

In some injection formulation examples such formulations are for any one of other roots of injections mentioned in this application or known in the art.

### Example 6

### Tablet Formulation

10 mg of any one of the compounds selected from the table 1 ab and Table 1c or composition, comprising such compound or its orally bioavailable form or delivery device or agent providing oral bioavailability, 50 mg microcrystalline cellulose, 10 mg potato or modified starch, 10 mg polyvinylpyrolidone, 10 mg methylcellulose, 25 mg dibasic calcium phosphate, 2 mg magnesium stearate, and 1.92 g Hydroxypropylmethylcellulose are prepared in a tablet formulation.

### Example 7

### Injection Formulation II

10 mg of any one of the compounds selected from Table 1 ab and Table 1c is provided in a vial in a sterile form for reconstitution as a suspension for subcutaneous injection or reconstitution as a solution with further dilution for intravenous infusion and 100 mg of mannitol as a sterile lyophilized powder.

### Example 8

### Tablet Formulation II

Any one of the compounds selected from the table 1 ab and Table 1c-20 mg, Ludipress −100 mg, Kollidon CL-10 mg, Magnesium stearate-10 mg and Aerosil-5 mg are prepared in a tablet formulation.

### Example 9

### Treatment for Frailty Reduction

C57BL/6J male mice aged 60 weeks (15 months) are ordered from Jackson Laboratories.

Animals are allowed to acclimate to the housing environment (the animal holding room) for 3 days prior to the initiation of the study. Animals are housed individually to avoid fighting. The animals have ad libitum access to standard diet (2018, Global 18% Protein Rodent Diet from Envigo++++, San Diego, CA) and acidified water (pH 2.5-3.0) throughout the study period. The bedding material is hardwood chips (Sani-Chips, Cat #7115, Envigo++++, CA, USA) and is changed Bi-weekly.

Four days before treatment start blood samples are collected for CBC analysis. CBC test parameters (Table 2) are used for DFI calculation for each animal. Animals are stratified in 3 groups based on DFI: control (n=48), rapamycin (n=48), and any one of the compound selected from the table 1 ab and Table 1c or composition, comprising such compound (n=48). Thus, at the beginning of the experiment, there is no statistically significant difference between all groups.

Rapamycin is prepared as 40 mg/mL stock solution in DMSO and stored at −20 C. Stock solutions are diluted to 1.2 mg/ml. Final formulation-5% Tween-80, 5% PEG-400, 3% DMSO (from stock solution) in water.

Any one of the compounds selected from the table 1 ab and Table 1c or composition, comprising such compound is prepared as 66.7 mg/mL stock solution in DMSO and stored at −20 C. Stock solutions are diluted to 2 mg/ml. Final formulation-5% Tween-80, 5% PEG-400, 3% DMSO (from stock solution) in water.

Control animals are treated with 5% Tween-80, 5% PEG-400, 3% DMS (from stock solution) in water.

Test/Control Agents are administered via intraperitoneal or intravenous or subcutaneous injection daily (Mon-Sun) or by any one of the protocols (dosage, regimen, root of administration) described in Example 1 or in other parts of this application. Animals' body weights are taken on Day-3 for randomization purposes and animals are monitored by weekly body weights throughout the entire study duration. Detailed clinical observations are performed weekly and recorded.

Every two weeks blood samples are collected and analyzed on hematology analyzer. The blood (120 μL) is collected into EDTA tubes via submandibular or facial vein using a lancet.

The mean value of bioage, frailty index or dFI in treatment group is less in comparison to the control group. At least one aging hallmark in the treatment group is reversed or alleviated. At least one parameter measured as shown in Example 1 indicates anti-aging effect or senolytic effect of compound of this application.

Alternatively, compound selected from the table 1 ab and Table 1c or composition, comprising such compound is dissolved in DMSO to 10 mM stocks and used with minimum 1% DMSO (for 10 micro M final measurements) and with 2% DMSO (for 200 micro M final measurements) or compounds are dissolved in water to 200 micro M stock solution. The stock solution is then used for further tests.

Alternatively, compound selected from the table 1 ab and Table 1c or composition, comprising such compound is dissolved in sterile water to 10 mM stocks or compounds are dissolved in water to 200 micro M stock solution. The stock solution is then used for further tests.

In another example, The compound selected from the table 1 ab and Table 1c is administered to groups of mice (dosages for respective groups: 0.1 mg/kg—for group #1, 1 mg/kg—for group #2, 10 mg/kg—for group #3, 100 mg/kg—for group #4 and 1 g/kg—for group #5) intraperitoneally from weeks 12-13 and weeks 14-15.

In yet another example, the compound, selected from the table 1 ab and Table 1c or composition, comprising such compound is administered in a dosage 20 times less than its LD50 by intraperitoneal injection.

In yet another example, the compound, selected from the table 1 ab and Table 1c or composition, comprising such compound is administered in a Maximum Tolerated dose by intraperitoneal injection.

In yet another example, the compound selected from the table 1 ab and Table 1c is administered by intraperitoneal injection (IP) in a doses: group #1-1 mg/kg, group #2-10 mg/kg, group #3-100 mg/kg.

TABLE 2
Complete blood counts parameters measured in the experiments,
which are used for calculation of marker of biological age.

| CBC parameter | Units |
|---|---|
| Granulocytes (neutrophils) | K/uL |
| Granulocytes | % |
| Hemoglobin | g/dL |
| Hematocrit | % |
| Lymphocytes | K/uL |
| Lymphocytes | % |
| Mean corpuscular hemoglobin | pg |
| Mean corpuscular hemoglobin | g/dL |
| concentration | |
| Mean corpuscular volume | fL |
| Platelets | K/uL |
| Red blood cells | M/uL |
| White blood cells | K/uL |

### dFI Correlates to Age

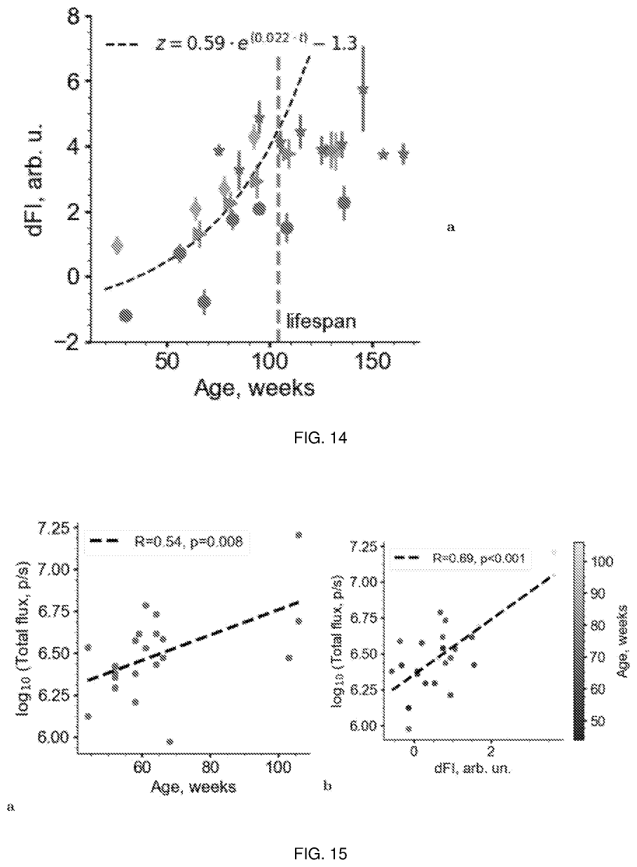

Dynamical frailty index (dFI) as a function of age in the test experiment is shown in FIG. 14: MA0071 (males, diamonds), MA0071 (females, circles) and MA0072 (triangles). The black curved dashed line is the exponential fit in the age groups younger than the average lifespan of NIH Swiss mice (indicated by the dashed vertical line). Stars mark the average dFI in age-matched groups of frail animals from the MA0073 cohort. All data are presented as mean±SEM.

dFI is Associated with the Number of Senescent Cells

Total flux (TF) in log scale representing p16-dependent luciferase reporter activity as a quantitative indicator of senescent cells is shown in FIG. 15: statistically significant correlations with age (a) and with dFI (b) in old mice (>50 weeks).

dFI is Associated with the Remaining Lifespan

TABLE 3
dFI is associated with the remaining lifespan

| | M (26 w) | F (26 w) | M (52 w) | F (52 w) | M (78 w) | F (78 w) |
|---|---|---|---|---|---|---|
| Cohort 1, animals | 98 | 125 | 155 | 188 | 167 | 150 |
| dF1 | −0.31 (1.68e−03) | −0.18 (4.77e−02) | −0.38 (1.35e−06) | −0.23 (1.37e−03) | −0.41 (5.12e−08) | −0.28 (5.23e−04) |
| Cohort 2, animals | 80 | 118 | 145 | 177 | 133 | 129 |
| dF1 | −0.34 (1.37e−03) | −0.21 (2.05e−02) | −0.38 (2.33e−06) | −0.25 (7.82e−04) | −0.32 (1.79e−04) | −0.31 (3.19e−04) |
| IGF1 | −0.31 (1.96e−03) | −0.17 (6.92e−02) | −0.19 (2.09e−01) | −0.11 (1.23e−01) | 0.03 (6.80e−01) | 0.05 (5.59e−01) |
| Body weight | −0.32 (1.61e−03) | −0.18 (3.34e−02) | −0.25 (2.25e−03) | −0.24 (9.71e−04) | 0.03 (0.92e−01) | 0.04 (6.78e−01) |

Spearman's rank-order correlation values and the corresponding p-values (in parentheses) for dFI with lifespan. Analysis is shown for two cohorts: Cohort 1 includes all animals with mortality data, Cohort 2 includes the subset of animals from Cohort 1 for which IGF1 measurements were available. Significant correlations (p<0.05) are highlighted in bold.

dFI correlates with Physiological Frailty index

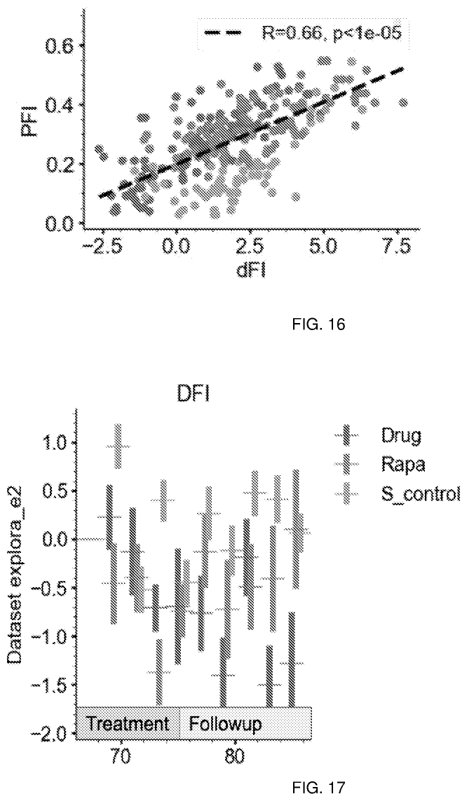

Correlation of dFI with the physiological frailty index (PFI) from Avchaciov et al., (2020) is shown in FIG. 16 and FIG. 17 shows the predicted change in dFI under compound treatment.

### Example 10

### Use for Anti-Aging Treatment in Humans

In some examples, a composition comprising any one of the compounds selected from Table 1 ab and Table 1c is administered orally.

In another examples, composition comprising any one of the compounds selected from Table 1 ab and Table 1c is administered IV.

In another examples, composition comprising any one of the compounds selected from Table 1 ab and Table 1c is administered by the root and in a formulation selected from this application.

In some examples, a composition comprising any one of the compounds selected from Table 1 ab and Table 1c is administered in the dosage of 0.1 mg. In some examples, a composition comprising any one of the compounds selected from Table 1 ab and Table 1c is administered in the dosage of 1 mg. In some examples, a composition comprising any one of the compounds selected from Table 1 ab and Table 1c is administered in the dosage of 10 mg. In some examples, a composition comprising any one of the compounds selected from Table 1 ab and Table 1c is administered in the dosage of 25 mg. In some examples, a composition comprising any one of the compounds selected from Table 1 ab and Table 1c is administered in the dosage of 50 mg. In some examples, a composition comprising any one of the compounds selected from Table 1 ab and Table 1c is administered in the dosage of 100 mg. In some examples, a composition comprising any one of the compounds selected from Table 1 ab and Table 1c is administered in the dosage of 250 mg. In some examples, a composition comprising any one of the compounds selected from Table 1 ab and Table 1c is administered in the dosage of 500 mg. In some examples, a composition comprising any one of the compounds selected from Table 1 ab and Table 1c is administered in the dosage of 1000 mg.

In yet another example, the compound selected from the table 1 ab and Table 1c or composition, comprising such compound is administered by the root of administration and in the highest dosage as it was in Phase 1 clinical trial of such compound.

In yet another example, the compound selected from the table 1 ab and Table 1c or composition, comprising such compound is administered by the root of administration and in the dosage selected from the group of dosages used in Phase 1 (if any) clinical trial of such compound.

In yet another example, the compound selected from the table 1 ab and Table 1c or composition, comprising such compound is administered by the root of administration and in the highest dosage as it was in Phase 2 (if any) clinical trial of such compound.

In yet another example, the compound selected from the table 1 ab and Table 1c or composition, comprising such compound is administered by the root of administration and in the lowest dosage as it was in Phase 2 (if any) clinical trial of such compound.

In yet another example, the compound selected from the table 1 ab and Table 1c or composition, comprising such compound is administered by the root of administration and in the highest dosage as it was in Phase 2 (if any) clinical trial of such compound.

In yet another example, the compound selected from the table 1 ab and Table 1c or composition, comprising such compound is administered by the root of administration and in the dosage as it was approved for other indication.

In yet another example, the compound selected from the table 1 ab and Table 1c or composition, comprising such compound is administered in its Maximum Tolerated Dose.

In yet another example, the compound selected from the table 1 ab and Table 1c or composition, comprising such compound is administered in dosage corresponding to 75% of its Maximum Tolerated Dose. In yet another example, the compound selected from the table 1 ab and Table 1c or composition, comprising such compound is administered in dosage corresponding to 50% of its Maximum Tolerated Dose. In yet another example, the compound selected from the table 1 ab and Table 1c or composition, comprising such compound is administered in dosage corresponding to 25% of its Maximum Tolerated Dose. In yet another example, the compound selected from the table 1 ab and Table 1c or composition, comprising such compound is administered in dosage corresponding to 10% of its Maximum Tolerated Dose. In yet another example, the compound selected from the table 1 ab and Table 1c or composition, comprising such compound is administered in dosage corresponding to 5% of its Maximum Tolerated Dose. In yet another example, the compound selected from the table 1 ab and Table 1c or composition, comprising such compound is administered in dosage corresponding to 1% of its Maximum Tolerated Dose. In yet another example, the compound selected from the table 1 ab and Table 1c or composition, comprising such compound is administered in dosage corresponding to 0.1% of its Maximum Tolerated Dose. In yet another example, the compound selected from the table 1 ab and Table 1c or composition, comprising such compound is administered in dosage corresponding to 0.01% of its Maximum Tolerated Dose.

In some examples, the compound selected from the table 1 ab and Table 1c or composition, comprising such compound is administered in the regimen as it was administered at the highest phase of its clinical trials known in the art.

In yet another example, the dosage is calculated to get the dosage selected from the group consisting of: 0.5 mg, 0.2 mg, 0.05 mg, 0.005 mg, 0.001 mg, 0.1 mg, 1 mg, 5 mg or 10 mg of any one of the compounds selected from the Table 1 ab and Table 1c per 1 kg of body weight.

The compound selected from the table 1 ab and Table 1c or composition, comprising such compound is optionally administered orally, intravenously, intra-arterially or intra-coronary arterially.

In some examples, the compound selected from the table 1 ab and Table 1c or composition, comprising such compound is administered once

In some examples, the compound selected from the table 1 ab and Table 1c or composition, comprising such compound is optionally administered in at least one of the dosages listed in this application in the regimen selected from the group consisting of: once, one time a week during one month, one time a week during two months, one time a week during 3 month, daily during 1 week, daily during 1 month, daily during 6 months or daily during 12 months, every 12 hours lifelong, 1 week daily having 1 week off the drug lifelong, 1 week daily having 1 month off the drug lifelong, 1 month daily having 1 year off the drug lifelong.

In some examples, the treatment is optionally repeated when the biological age of the subject returns to the level when the first treatment was administered.

In some examples, the treatment is optionally repeated when the symptom of aging or aging related disease or condition has returned to the level existed just before the first treatment.

In yet another example, composition comprising any one of the compounds selected from the table 1 ab and Table 1c or composition, comprising such compound is administered orally every 24 hours for 1 week, 1 month and 3 months and is repeated when the biological age of the subject returns to the level when the first treatment had been administered.

In yet another example, composition comprising any one of compound selected from the table 1 ab and Table 1c or composition, comprising such compound is administered orally every 12 hours for 1 week, 1 month and 6 months and is repeated when the biological age of the subject returns to the level when the first treatment had been administered.

In yet another example, composition comprising any one of the compounds selected from the table 1 ab and Table 1c or composition, comprising such compound is administered by the protocol selected from this application.

Optionally, the compounds are administered to subject of 75 kg weight in a tablet formulation, where the tablet comprises: any one of the compounds selected from Table 1 ab and Table 1c—in the dosage selected from the group comprising: 1 mg, 10 mg, 50 mg, 100 mg, 200 mg, 500 mg, 750 mg, 1 g and 1 Ludipress-17 mg, Kollidon CL-2 mg, Magnesium stearate-2 mg, Aerosil-1 mg.

Optionally, the patient is healthy, aged or has at least one aging related disease, disorder or decline. For example, for the component of frailty “hands grip”, the hands grip force is measured by the means known in the art. For example, for cognitive decline a test such as “Reaction time” is used: subjects complete a timed test of symbol matching, similar to the common card game ‘Snap’ hereafter referred to as reaction time (RT) (http://biobank.ctsu.ox.ac.uk/crystal/field.cgi?id=20023). The score on this task is the mean response time in milliseconds across trials which contained matching pairs. See for details e.g. Cognitive Test Scores in UK Biobank: Data Reduction in 480,416 Participants and Longitudinal Stability in 20,346 Participants (Donald M. Lyall et al., 2016). After the treatment, the force of hands grip is increased and the time of mean response time in test of symbol matching is decreased.

Any one of the compounds selected from Table 1 ab and Table 1c is used to prevent and treat frailty. The frailty index of a subject can be measured by many ways known in the art, including but not limited to as described in Rockwood et al., 2005, Searle et al. 2008 and Rockwood et al., 2017.

Other anti-aging effects are measured in many ways known in the art including but not limited to those described in Justice et al., 2016, an effect against particular diseases—by the methods, tests and equipment known to the expert in the particular disease etc. The list of the parameters that can be controlled after the treatment, as well as the regimen of such control (by default-1 month, 3 months, 6 months, 12 months, 18 months, 24 months, 5 years, 10 years, 20 years) after the treatment but can be changed), and methods and equipment for control are known to the experts in the field of specific aging related disease, disorder or decline. Other parameters that are improved by the treatment are healthspan and lifespan.

Anti-aging effects are also identified as a change of at least one of the biomarker of aging or aging related decline into more juvenile state or the delay in progression into more biological elder state in comparison with untreated.

Anti-aging effects are also identified as an alleviation of at least one symptoms of aging or aging related disease or condition.

### Example 11

### Mice Model of Metabolic Syndrome and Animal Models for Other Diseases

### Metabolic Syndrome

Metabolic syndrome is an age related disease. To confirm the effect of any one of the compound selected from the table 1 ab and Table 1c or composition, comprising such compound on an animal model of metabolic syndrome, mice with diet induced obesity are used. 15 months old C57BL/6J mice are divided into groups (n=50): one group receives standard laboratory chow (LabDiet JL 6% Oval, Cat #5K0Q), other groups are fed on 60% fat chow (DIO Rodent Purified Diet w/60% Energy From Fat, Cat #58Y1). Groups on high fat diet are treated with vehicle and any one of the compounds selected from the table 1 ab and Table 1c or composition, comprising such compound, PO administration, every 6 h at 0.05 mg/kg, 0.5 mg/kg, 1 mg/kg or 10 mg/kg.

In some examples, the mice are treated by the protocol (dosage, regimen and root of the administration) described in Example 1.

Optionally, any one of the compound selected from the table 1 ab and Table 1c or composition, comprising such compound or any other compound of this disclosure is injected, optionally 0.001 mg/kg, 0.025 mg/kg, 0.25 mg/kg, 0.01 mg/kg, 0.5 mg/kg, 1 mg/kg, 2.5 mg/kg, 5 mg/kg, 10 mg/kg, 25 mg/kg or 50 mg/kg, 2 times a day by IV. In some examples, in a same dosage subcutaneous or intraperitoneal injection can be done.

In other example, injection of any one of the compound selected from the table 1 ab and Table 1c or composition, comprising such compound or any other compound of this disclosure in the same dosage as shown above in this example is administered subcutaneously or orally.

Optionally, the animals are treated once, or daily: for 1 week, for 2 weeks, for 1 month, for 3 months or for 6 months.

After 6 weeks or (in other example) after the end of treatment animal weight and fasting blood glucose, insulin, triglycerides, and leptin levels are assessed and glucose/insulin tolerance tests are performed. In prophetic example animals treated with the agent or combination of this application will have healthier levels of the parameter selected from the group consisting of: weight, fasting blood glucose, insulin, triglycerides, leptin levels and glucose/insulin tolerance.

### Example 12

### Example 12.1 Animal Experiment on Aging Related Diseases and Disorders

This experiment is conducted on a relevant species (for example, mice, rat etc.) and disease model of a particular aging related disease, disorder or decline.

As non-limiting examples, it could be animal models known in the art, e.g. models of disease or condition selected from the group consisting of: Alzheimer's disease, Parkinson's disease, cataracts, macular degeneration, glaucoma, atherosclerosis, acute coronary syndrome, myocardial infarction, stroke, recovery after stroke, PSD-95 decrease, hypertension, idiopathic pulmonary fibrosis (IPF), chronic obstructive pulmonary disease (COPD), type 1 diabetes, obesity, fat dysfunction, coronary artery disease, cerebrovascular disease, periodontal disease, cancer treatment-related disability, chemotherapy treatment-related disability, chemotherapy treatment-related frailty, frailty, radiotherapy and other radiation related disability, chemotherapy treatment-related frailty, cancer treatment-related atrophy, cancer treatment-related fibrosis, brain injury, heart injury, and therapy-related myelodysplastic syndrome, accelerated aging, accelerated aging disease, Hutchinson-Gilford progeria syndrome, Werner syndrome, Cockayne syndrome, exroderma pigmentosum, ataxia telangiectasia, Fanconi anemia, dyskeratosis congenital, aplastic anemia, idiopathic pulmonary fibrosis, atherosclerosis, cardiovascular disease, adult cancer, arthritis, cataracts, osteoporosis, type 2 diabetes, diabetes, hypertension, neurodegeneration, stroke, atrophic gastritis, osteoarthritis, NASH, camptocormia, chronic obstructive pulmonary disease, coronary artery disease, dopamine dysregulation syndrome, metabolic syndrome, effort incontinence, Hashimoto's thyroiditis, heart failure, late life depression, immunosenescence, myocardial infarction, acute coronary syndrome, sarcopenia, sarcopenic obesity, senile osteoporosis and urinary incontinence or any other disease or disorder mentioned in this application or known in the art age related disease or condition.

In yet another example, the animal models for senescence associated diseases and conditions are described in patent application EP3099380A1 “Methods and compositions for killing senescent cells and for treating senescence-associated diseases and disorders”, optionally the dosage, regimen and root of administration is selected from the Example 1.

In yet another example, the dosage and regimen are taken as defined as expert in the field.

In yet another example, the protocol of compound selected from the table 1 ab and Table 1c is taken from the Example 1.

The monitoring of efficacy of the compound selected from the table 1 ab and Table 1c tested in such model can be trivially done using the methods known for the respective animal model.

### Example 12.2 Stroke Recovery

### Humans

Patients who had ischaemic stroke and hemiplegia or hemiparesis, have Fugl-Meyer motor scale (FMMS) scores of 55 or less or any other post-stroke syndromes known to the experts in the field are studied in this example.

In one example, the compound selected from the table 1 ab and Table 1c is used by in the formulation, root of administration, dosage and regimen selected from the Example 10.

The primary outcome measure is the change on the FMMS between day 0 and day 90 after the start of the study drug in comparison with placebo. Participants, carers, and physicians assessing the outcome are masked to group assignment. 1000 patients are randomly assigned to Treatment (n=500) or placebo (n=500), and 1000 are included in the analysis. FMMS improvement or at least one other health parameter worsened after the stroke at day 90 is be greater in the Treatment group than in the placebo group. In other example stroke recovery can be evaluated by the Behavior Tests

### Mice

In one example, any one of the compounds selected from the table 1 ab and Table 1c or composition, comprising such compound or placebo for 3 months are administered starting 5-10 days after the onset of stroke by the route, regimen and dosage selected from the described in any one of the Examples 1, 4, 9, 10, 11, 12.1 or 12.2 or by the route, regimen and dosage as it is used for its primary indication or by the route, regimen as it is used for its primary indication, but the dosage is about selected from the group consisting of: 0.1; 0.5; 2 times more, 5 times more, 10 times, 50 times more in comparison with the effective dosage of its primary indication.

In other example stroke recovery can be evaluated by the Behavior Tests

To assess functional outcomes, beam walking and gait analysis can be used to evaluate the motor and sensorimotor asymmetries, respectively. The beam walking test canb e performed blindly before stroke and were repeated at 1, 7, 14, 28, and 42 days after stroke as described previously (Fan et al., 2014). Gait analysis can be performed with an automated computer-assisted method (CatWalk™, Noldus Information Technology, Wageningen, The Netherlands) in accordance with the manufacturer's instructions and published procedures (Balkaya et al., 2013; Caballerogarrido et al., 2015). In brief, mice are allowed to move freely across an elevated 1.3 m long glass platform. Footprints are captured by a high-speed camera and analyzed on the computer using the Catwalk XT 8.1 software. In some examples, The animals are trained for 3 days before surgery and tested at days 1, 7, 14, 28, 42 after stroke. The max contact area and the print length are used to evaluate the influence of the Compound selected from the table 1 ab and Table 1c treatment to the mice after stroke.

Compound selected from the table 1 ab and Table 1c Increases Neuronal Regeneration and Improved Functional Recovery after Stroke

To determine whether Compound selected from the table 1 ab and Table 1c affects NPCs differentiation, one can treat mice with either vehicle or Compound selected from the table 1 ab and Table 1c at 7-13 days and examine brains at 42 days after stroke. The results indicate that Compound selected from the table 1 ab and Table 1c increases the number of BrdU+ cells and BrdU+/NeuN+ cells in the peri-infarct cortex at 42 days. The number of BrdU+/GFAP+ cells are comparable between control and Compound selected from the table 1 ab and Table 1c-treated mice. These data suggests that Compound selected from the table 1 ab and Table 1c promotes regeneration of neuronal cells. Measurements of motor activity by the beam walking test and sensorimotor performance by the CatWalk gait indicate no differences in behavioral deficits in Compound selected from the table 1 ab and Table 1c group before treatment compared with vehicle-treated group at 1-7 days after stroke. However, Compound selected from the table 1 ab and Table 1c-treated mice has profound improvement in long-term motor activity and sensorimotor performance.

More details on the protocol is available in the art, e.g. in Lu L, Bai X, Cao Y, et al. Growth Differentiation Factor 11 Promotes Neurovascular Recovery After Stroke in Mice. Front Cell Neurosci. 2018; 12:205. Published 2018 Jul. 16. doi:10.3389/fncel.2018.00205

### Example 13

### Mice Experiment on Frailty and Lifespan

In this experiment, the dosage and regimen as shown in Example 1 is used to show that any one of the compounds selected from the table 1 ab and Table 1c or composition, comprising such compound of this application has an anti-aging effect.

Effect of the treatment on biological age is estimated based on changes in standard blood count (Antoch et al, 2017), DNA methylation (Stubbs et al., 2017; Horvath, 2013), lifespan (Harrison et al., 2009), and healthspan assessed by frailty index. A frailty index is created by counting the accumulation of deficits in health across many systems in the body. Deficits measured to construct a frailty index include a large number of health-related variables related to the function of systems that are known to change with age in both human and animal models (Parks et al., 2012). These variables provide information about the following: (a) activity, including distance moved, velocity of movement and rearing frequency; (b) hemodynamic status, including heart rate, systolic and diastolic blood pressure; (c) body composition, including body mineral content, percent body fat and percent lean tissue; and (d) basic metabolism and organ function, including serum electrolyte levels, hematocrit and urea levels clinical signs, symptoms, diseases, and laboratory and radiographic abnormalities.

Cognitive score is assessed using Barnes maze, activity chamber and fear-conditioning tests.

The Barnes maze test consists of a large circular maze containing 40 holes and elevated approximately 40 cm above the floor. The escape hole consists of a long PVC elbow joint connector that was similar in texture and color to the maze. The escape hole position is fixed within a day but changed for each successive day of testing. For each trial within a day, the starting location for the mouse is rotated relative to the escape hole position. Between each trial, the maze and escape hole are thoroughly cleaned to remove any cues that might affect performance in subsequent trials. With overhead illumination and a persistent 2 kHz tone, mice are given 90 s for each trial to identify the escape hole by jumping in or identifying the hole with extended/overt head pokes.

An activity chamber (Med Associates Inc., St. Albans, VT) is used to evaluate general locomotor activity and exploratory behavior in a novel environment. It consists of a square arena located in a sound-attenuated chamber. Mice are placed in the center of the arena and tracked by an automated tracking system with three planes of infrared detectors during a 10-min trial. Before each trial, the surface of the arena is cleaned with 10% ethanol. Distance moved, velocity, and rearing activity are measured.

To assess pain sensitivity to the foot-shock apparatus used for fear-conditioning, each mouse is placed in the foot-shock chamber with a front-mounted camera to visualize various pain sensitivity parameters, which encompasses four clearly defined behaviors, ‘flinch’, ‘run’, ‘vocalization’, and ‘two-paw jump’. One-second shocks of various intensities are delivered each spaced by 30 s of recovery time. Thresholds reflected the minimum shock intensity at which each behavior was observed using the front-mounted camera video.

### Example 14

### COVID-19 Experiments

### In Vitro Experiments

To investigate anti-SARS-CoV-2 activity of any one of the compounds selected from the table 1 ab and Table 1c or composition, comprising such compound in vitro, Vero E6 cells (ATCC-1586) are used. The cells are grown in Dulbecco's Modified Eagle Medium (DMEM) (Sigma Aldrich, Boston, MA, USA) supplemented with 5% fetal bovine serum (Logan, UT, USA) at 37° C. and 5% CO2.

Cytotoxicity of the compounds in Vero cells is determined by the CCK8 assay.

For the treatment study, Vero cells are seeded into 96-well plates at a density of 1×104 cells/well and grown for 24 hours. Cells are then infected with SARS-CoV-2 at a multiplicity of infection (MOI) of 0.01 (100 PFU/well) for 2 hours at a temperature of 37° C. Virus input is washed with DMEM and the cells are then treated with medium containing test compounds at various concentrations (3-fold dilutions at seven different concentrations starting from CC50/10) for 24 or 48 hours.

For the prophylactic study, Vero cells are grown as described above and are pretreated with various concentrations of the compounds ((3-fold dilutions at seven different concentrations starting from CC50/10) for 2 hours, after that drug containing medium is removed and SARS-CoV-2 virus-containing medium is added (as described for the treatment study) for 2 hours. Following this, the virus-containing medium is removed and replaced with fresh medium that does not contain drugs or viruses.

Efficacies are evaluated by quantification of viral copy numbers in the cell supernatant via quantitative real-time RT-PCR (qRT-PCR) (as described in [Huang C, Wang Y, Li X, et al. Clinical features of patients infected with 2019 novel coronavirus in Wuhan, China. Lancet. 2020. 395 (10223): 497-506]) and confirmed with visualization of virus nucleoprotein (NP) expression through immunofluorescence microscopy at 48 h post infection.

Based on this data cytotoxicity (CC50) and virus replication and infection inhibition (EC50) is determined. EC50 is lower than CC50.

### In Vivo Experiments

To investigate the anti-COVID-19 activity of the any one of the compounds selected from the table 1 ab and Table 1c or composition, comprising such compound in vivo, BALB/C mice are infected with SARS-CoV (MA15). All infections are performed in an animal biosafety level 3 facility using appropriate practices, including a HEPA-filtered bCON caging system, HEPA-filtered powered air-purifying respirators (PAPRs), and Tyvek suiting. All animals are grown to 10 weeks of age prior to use in experiments. The animals are anesthetized using a mixture of xylazine (0.38 mg/mouse) and ketamine (1.3 mg/mouse) in a 50-μl total volume by intraperitoneal injection. The mice are inoculated intranasally with 50 μl of either PBS or 1×105 PFU of rMA15 SARS-CoV, after which all animals are monitored daily for weight loss. Test compounds are administered-every day via i.p route in the dosage which is used for the successful efficacy tests made at the same or alike animal for such drug's initial indication
or by the route, regimen and dosage which is used for the efficacy tests made at the same animal.
   In another example, the protocol of compound administration is selected from the Example 1.
Mice are euthanized at days 2, 5, or 9 post infection, and lung tissue was harvested for further analysis.

Fifty percent tissue culture infectious dose (TCID50) values from SARS-CoV (MA15)-infected lungs is calculated by infecting multiple replicates of Vero E6 cells plated on 96-well plates. Serial dilutions (1:5) are performed for the virus-containing lung lysates in Vero E6 culture medium (complete MEM) such that at least the last two dilutions had no detectable virus in any of the replicates. The infection proceeds for 3 days before cells are fixed with 4% PFA and stained with 0.05% crystal violet in 20% methanol. The TCID50 is calculated using the formula log 10 TCID50=Xp+ (0.5×D)−(D×Sp), where Xp is the last sample where all sample replicates are positive, D is the serial dilution log, and Sp is the sum of the proportion of replicates at all dilutions where positive values are seen (starting with the Xp dilution).

### Example 15

### Cancer Supportive Care Treatment

### 15.1 Preclinical

Animals (in some examples—mice) are treated with a dose corresponding to LD10 or to LD25 of a chemotherapy agent, for example but not limited to one selected from the following group consisting of: aminoglutethimide, amsacrine, anastrozole, asparaginase, bcg, bicalutamide, bleomycin, bortezomib, buserelin, busulfan, campothecin, capecitabine, carboplatin, carfilzomib, carmustine, chlorambucil, chloroquine, cisplatin, cladribine, clodronate, colchicine, cyclophosphamide, cyproterone, cytarabine, dacarbazine, dactinomycin, daunorubicin, demethoxyviridin, dichloroacetate, dienestrol, diethylstilbestrol, docetaxel, doxorubicin, epirubicin, estradiol, estramustine, etoposide, everolimus, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, lenalidomide, letrozole, leucovorin, leuprolide, levamisole, lomustine, lonidamine, mechlorethamine, medroxyprogesterone, megestrol, melphalan, mercaptopurine, mesna, metformin, methotrexate, mitomycin, mitotane, mitoxantrone, nilutamide, nocodazole, octreotide, oxaliplatin, paclitaxel, pamidronate, pentostatin, perifosine, plicamycin, pomalidomide, porfimer, procarbazine, raltitrexed, rituximab, sorafenib, streptozocin, sunitinib, suramin, tamoxifen, temozolomide, temsirolimus, teniposide, testosterone, thalidomide, thioguanine, thiotepa, titanocene dichloride, topotecan, trastuzumab, tretinoin, vinblastine, vincristine, vindesine, vinorelbine, ABT-263, dexamethasone, 5-fluorouracil, PF-04691502, romidepsin, and vorinostat (SAHA) by the route known the art as effective for anti-cancer use for such animals. Frailty Index (FI) is measured as described in Animals are left for one month of recovery.

FI measurement is repeated. Animals are stratified in two groups: the control and the treatment groups. Treatment by a compound selected from Table 1 ab and Table 1c is performed for 4-8 weeks followed by FI measurement, followed by another dose of LD10 or LD25 of chemotherapy agent.

In some examples, compound selected from Table 1 ab and Table 1c is administered by the root of administration, dosage, formulation selected from the example 1.

Survival is evaluated. Animals in treatment group demonstrate better survival.

### Clinical

Patients undergoing chemotherapy are enrolled. After the first round of chemotherapy FI is evaluated. Patients are stratified in placebo and treatment groups. Treatment groups receive a compound selected from the Table 1 ab and Table 1c or from any other compounds described herein. Patients receive the second round of chemotherapy treatment. Rate of withdrawal and dose reduction is evaluated. Patients in the treatment group have lower rates of withdrawal and dose reduction.

In some examples, Any one of the compounds selected from Table 1 ab and Table 1c or any other compound described herein is administered by PO administration, every 24 h in a dosage of 0.001 mg/kg, 0.005 mg/kg, 0.01 mg/kg, 0.05 mg/kg, 0.5 mg/kg, 1 mg/kg or 10 mg/kg.

In some examples, Any one of the compounds selected from Table 1 ab and Table 1c or any other compound described herein may be administered with food. For example, the compound could be mixed with feed at a dosage of 0.01 mg/kg, 0.1 mg/kg, 1 mg/kg, 5 mg/kg, 10 mg/kg, 20 mg/kg or 50 mg/kg.

In some examples, Any one of the compounds selected from Table 1 ab and Table 1c or any other compound described herein may be administered by IV injection, for example 0.025 mg/kg, 0.25 mg/kg, 0.01 mg/kg, 0.5 mg/kg, 1 mg/kg, 2.5 mg/kg, 5 mg/kg, 10 mg/kg, 25 mg/kg or 50 mg/kg−1 time a day.

Any one of the compounds selected from Table 1 ab and Table 1c or any other compound described herein may be administered subcutaneously or orally in the same dosage as shown above in this example.

In some examples, the protocol of administration is selected from the Example 10.

Patients in the treatment group have lower rates of withdrawal and dose reduction.

15.2 Use Together with Immunotherapy

In some examples, any one of the compounds selected from Table 1 ab and Table 1c, or composition, comprising such compound, other compound or composition disclosed herein can be used or administered prior the immunotherapy in the formulation, dosage and regimen as shown in any one of the Examples 10 above or any other part of this disclosure or as defined by practitioner.

In some other examples, any one of the compounds selected from Table 1 ab and Table 1c or composition, comprising such compound can be used or administered concomitantly with the immunotherapy in the formulation, dosage and regimen as shown in Examples 10.

In some other examples, any one of the compounds selected from Table 1 ab and Table 1c or composition, comprising such compound can be used or administered as defined by practitioner.

In one of the many possible examples, the compound of this disclosure is administered prior the treatment by the immunotherapy in regimen and dosage effective for changing at least one of aging biomarkers of the patient in younger or more healthy state, and immunotherapy is used soon after such biomarker was improved.

In yet another of the many possible examples, the biomarker (or aging related biomarker) known for practitioners as being predictive for the efficacy of the particular immunotherapy is measured. In case if there is a therapeutic need to improve a predicted efficacy of such immunotherapy, the compound of this invention is administered in the formulation, dosage and regimen as shown in Example 10 or in any other part of this disclosure or as defined by practitioner or as needed to achieve efficacy of the corresponding immunotherapy or as needed to change such biomarker to the level predictive that the corresponding immunotherapy will be effective or more effective.

### Example 16 Direct Anti-Cancer Action

### Primary Adenocarcinoma NSCLC Xenograft Model

Tumor tissue is excised from a patient with a poorly differentiated adenocarcinoma. This tumor tissue is implanted subcutaneously in the flank of a SCID mouse and passaged twice before compound testing. For compound testing passage-two tumor tissue is implanted subcutaneously in the flank of SCID mice and tumors grown to a volume of about 200 mm3.

A compound selected from Table 1 ab and Table 1c is dosed as a solution alone in the dosage, regimen and route of administration for compounds is as in Example 1 or on 4 consecutive days per weekly cycle (qd4, dosed on days 1, 2, 3 and 4 each week). A control group receives vehicle alone (10% Vitamin E TPGS in water, po qd4). All drug treatment is stopped on Day 28. Vehicle, and compound treatment groups are sacrificed and the remainder monitored for a further 40 days to assess tumor re-growth. Tumor size in treatment group are less and other cancer symptoms are alleviated. Additional details on this method are described in Kellar et al, 2015. Other models used to demonstrate anti-cancer activity are listed in Kellar et al., 2015.

### Example 17

### Treatment of P16-3MR Transgenic Mice (Prophetic)

This example describes an animal model useful for determining the capability of a compound selected from the table 1 ab and Table 1c to selectively kill senescent cells in vivo. The capability of the compound selected from the table 1 ab and Table 1c to remove senescent cells in vivo is determined in transgenic p16-3MR mice (see, e.g., International Application Publication No. WO2013/090645). The transgenic mouse comprises a p16^{Ink4a} promoter operatively linked to a trimodal fusion protein for detecting senescent cells and for selective clearance of senescent cells in these transgenic mice. The promoter, p16^{Ink4a}, which is transcriptionally active in senescent cells but not in non-senescent cells (see, e.g., Wang et al, J. Biol. Chem. 276:48655-61 (2001); Baker et al, Nature 479:232-36 (2011)), is engineered into a nucleic acid construct. 3MR (tri-modality reporter) is a fusion protein containing functional domains of a synthetic Renilla luciferase (LUC), monomeric red fluorescence protein (mRFP), and truncated herpes simplex virus (HSV)-1 thymidine kinase ((TK), which allows killing by ganciclovir (GCV) (see, e.g., Ray et al, Cancer Res. 64:1323-30 (2004)). The 3MR cDNA is inserted in frame with p16 in exon 2, creating a fusion protein containing the first 62 amino acids of pi 6, but not a full-length wild-type p16 protein, Insertion of the 3MR cDNA also result in the occurrence of a stop codon in the p19ARF reading frame in exon 2, thereby preventing full-length pi 9ARF expression from the BAC as well. The p16Ink4a gene promoter (approximately 100 kilobase pairs) is introduced upstream of a nucleotide sequence encoding a trimodal reporter fusion protein. Alternatively, a truncated pi 6Ink4a promoter may be used (see, e.g., Baker et al., Nature, supra; International Application Publication No. WO2012/177927; Wang et al, supra). Thus, the expression of 3MR is driven by the p16Ink4a promoter in senescent cells only. The detectable markers, LUC and mRFP permitted detection of senescent cells by bio luminescence and fluorescence, respectively. The expression of tTK permitted selective killing of senescent cells by exposure to the pro-drug ganciclovir (GCV), which is converted to a cytotoxic moiety by (TK. Transgenic founder animals, which have a C57B16 background, are established and bred using known procedures for introducing transgenes into animals (see, e.g., Baker et al, Nature 479:232-36 (2011)).

To determine the senolytic activity of an agent, such as the compound selected from the table 1 ab and Table 1c, female C57/BL6 p16-3MR mice are randomized into doxorubicin+the compound selected from the table 1 ab and Table 1c treated or doxorubicin only treated groups. Senescence is induced by intraperitoneal administration of doxorubicin at 10 mg/kg to the mice ten days prior to administration of the compound selected from the table 1 ab and Table 1c (Day −10).

In one example, the compound selected from the table 1 ab and Table 1c is administered intraperitoneally in different dosages (each for each treatment group): 0.1 mg/kg, 1 mg/kg, 10 mg/kg, 100 mg/kg and 1 g/kg daily from day 10 to day 24 post-doxorubicin treatment (Each Treatment group=90 mice). Control mice (doxorubicin treated) are injected with equal volumes of PBS (Group=30 mice).

In another example, The compound selected from the table 1 ab and Table 1c is administered by the root of administration and dosage selected from the Example 1 from day 10 to day 24 post-doxorubicin treatment (Group=90 mice).

Luminescence imaging (Xenogen Imaging system) is performed at Day 0 (i.e., 10 days post-doxorubicin treatment) as a baseline for each mouse (100% intensity).

Luminescence imaging of the mice is performed on day 7, 14, 21, 28, and 35 following the initiation of the compound selected from the table 1 ab and Table 1c treatment. Reduction of luminescence (L) is calculated as: L=(Imaging post—the compound selected from the table 1 ab and Table 1c treatment)/(Baseline Imaging) %. If L is greater than or equal to 100%, the number of senescent cells was not reduced. If L is less than 100%), then the number of senescent cells was reduced. Every mouse is calculated independently, and background is subtracted from each sample.

Experiments are performed to determine the effect of the compound selected from the table 1 ab and Table 1c treatment on expression of genes associated with senescence. Groups of female C57/BL6 p16-3MR are treated as described above. Three weeks after the end of the compound selected from the table 1 ab and Table 1c treatment (day 35), the doxorubicin treated mice (control) (N=30) and doxorubicin+WEHI-539-treated mice (N=60) are sacrificed. Skin and fat biopsies are collected for RNA extraction; fat biopsies are collected for detection of senescence-associated β-galactosidase; and lungs are flash frozen in cryoprotectant OCT media for eryostat sectioning.

R A is analyzed for mR A levels of endogenous senescence markers (e.g., p21, p16^{INK4a} (pi 6), and p53) and SASP factors (e.g., mmp-3 and IL-6) relative to actin mRNA (control for cDNA quantity) using the Roche Universal Probe Library for real-time PCR assay.

The frozen lung tissue is sectioned to 10 μM thickness and stained with primary rabbit polyclonal antibody against γH2AX (Novus Biologicals, LLC), which is a marker for double-strand breaks in cells (DNA damage). The sections are then stained with ALEXA FLUOR® dye-labeled secondary goat anti-rabbit antibody (Life Technologies) and counterstained with 4′,6-diamidino-2-phenylindole (DAPI) (Life Technologies). The number of positive cells is calculated using Image J image processing program (National Institutes of Health, see Internet at imagej.nih.gov/ij/index.html) and represented as a percentage of the total number of cells. Upon collection, fat biopsies are immediately fixed in 4% formalin and then stained with a solution containing X-gal to detect the presence of senescence-associated B-galactosidase (β-gal). Fat biopsies are incubated overnight at 37° C. in X-gal solution and are photographed the next day. Fat biopsies from untreated animals are used as a negative control (CTRL).

### Example 18

### Animal Model of Osteoarthritis

The two treatment studies are designed to determine the effect of removing senescent cells in an animal model of osteoarthritis.

Parallel studies can be performed. One study investigates the effect of eliminating senescent cells with the compound selected from the table 1 ab and Table 1c in 3MR mice. Mice should go under surgery to cut the anterior cruciate ligament of one rear limb to induce osteoarthritis in the joint of that limb. During week 2 post-surgery, seven 3MR mice groups (n=30 in each group) receive 0.01 μg, 0.1 μg, 1 μg, 2.5 μg, 5 μg, 10 μg and 100 μg of the compound selected from the table 1 ab and Table 1c to the operated knee by intra-articular injection, qd for 5 days, with a 2ªd treatment (the same dose of the compound selected from the table 1 ab and Table 1c qd for 5 days) during week 4 post-surgery. At the end of 4 weeks post-surgery, operated joints of the mice are monitored for presence of senescent cells, assessed for function, monitored for markers of inflammation, and underwent histological assessment. In a parallel study, C57BL/6J mice should go under surgery to cut the anterior cruciate ligament of one rear limb to induce osteoarthritis in the joint of that limb.

In some examples, During week 3 and week 4 post-surgery, seven 3MR mice groups (n=30 in each group) receive each group—particular dosage: 0.01 μg, 0.1 μg, 1 μg, 2.5 μg, 5 μg, 10 μg and 100 μg of the compound selected from the table 1 ab and Table 1c per operated knee by intra-articular injection, qod for 2 weeks.

In some other examples, the protocol of administration of compound selected from the table 1 ab and Table 1c is selected from the Example 1.

In some other examples, compound selected from the table 1 ab and Table 1c is administered During week 3 and week 4 post-surgery, the rest of the protocol of administration of compound selected from the table 1 ab and Table 1c is selected from the Example 1.

At the end of 4 weeks post-surgery, joints of the mice are monitored for presence of senescent cells, assessed for function, monitored for markers of inflammation, and underwent histological assessment.

Two control groups of mice are included in the studies performed: one group comprising C57BL/6J or 3MR mice that should undergo a sham surgery (n=3) (i.e., surgical procedures followed except for cutting the ACL) and intra-articular injections of vehicle parallel to the compound selected from the table 1 ab and Table 1c-treated group; and one group comprising C57BL/6J or 3MR mice that had undergone an ACL surgery and received intraarticular injections of vehicle (n=5) parallel to the compound selected from the table 1 ab and Table 1c-treated group.

RNA from the operated joints of mice from the compound selected from the table 1 ab and Table 1c treated mice are analyzed for expression of SASP factors (mmp3, IL-6) and senescence markers (pi 6). qRT-PCR is performed to detect mRNA levels. In some examples, the treatment with the compound selected from the table 1 ab and Table 1c clears senescent cells from the joint. RNA from the operated joints of mice are also analyzed for expression of type 2 collagen and compared with expression of actin as a control. The treatment with the compound selected from the table 1 ab and Table 1c in mice that have undergone osteoarthritis surgery drives collagen production as compared to untreated mice.

Function of the limbs are assessed 4 weeks post-surgery by a weight bearing test to determine which leg the mice favored. The mice are allowed to acclimate to the chamber on at least 3 occasions prior to taking measurements. Mice are maneuvered inside the chamber to stand with 1 hind paw on each scale. The weight that is placed on each hind limb is measured over a 3-second period. At least 3 separate measurements are made for each animal at each time point. The results are expressed as the percentage of the weight placed on the operated limb versus the contralateral unoperated limb. The untreated mice that have undergone osteoarthritis surgery favor the unoperated hind limb over the operated hind limb (4). In some examples, clearing senescent cells with the compound selected from the table 1 ab and Table 1c abrogates this effect in mice that have undergone surgery (V). In some examples, the compound selected from the table 1 ab and Table 1c abrogates this effect in mice that have undergone surgery (V

The function of the limbs is also assessed at 4 weeks post-surgery by hotplate analysis to show sensitivity and reaction to pain stimulus. In brief, a mouse is placed on a hotplate at 55° C. When placed on the hot surface of the plate, mice will lift their paws and lick them (paw-lick response) due to attainment of pain threshold. The latency period for the hind limb response (paw-lick response) is recorded as response time. The untreated mice that have undergone osteoarthritis surgery have an increased response time as compared to normal mice that have not been surgically altered. However, treatment of mice that have undergone osteoarthritis surgery with the compound selected from the table 1 ab and Table 1c decreases the response time in a significant manner.

Histopathology of osteoarthritis induced by ACL surgery illustrates that the proteoglycan layer is destroyed. The compound selected from the table 1 ab and Table 1c abrogated this effect.

in some examples, Clearing of senescent cells from the 3MR mice treated with the compound selected from the table 1 ab and Table 1c had the same impact on pathophysiology of osteoarthritis.

### Example 19

Any one of the treatment methods of humans mentioned in this application, wherein the protocol of administration of compound selected from the table 1 ab and Table 1c or composition, comprising such compound is selected from the Example 10, while the methods of efficacy control for the specific disease are used as described for this disease in this application or, if absent—as known in the art.

### Example 20

Effect Compound Selected from Table 1 ab and Table 1c in pulmonary disease Models

One animal model study assesses the effect of clearance of senescence cells in the transgenic mouse strain 3MR that has bleomycin induced lung injury. In the bleomycin injury model for idiopathic pulmonary fibrosis, mice develop lung fibrosis within 7-14 days after bleomycin treatment. Bleomycin is administered to anesthetized 6-8 week old 3MR mice by intratracheal aspiration (2.5 U/kg of bleomycin in 50 ρt PBS) using a microsprayer syringe (Penn-Century, Inc.) as described in Daniels et al. (2004, J. Clin. Invest. 114:1308-1316). Control mice are administered saline.

In one example, the day following bleomycin treatment, Compound selected from the table 1 ab and Table 1c in different dosages (0.01 mg/kg—for group #1, 0.1 mg/kg—for group ##2, 1 mg/kg—for group #3, 10 mg/kg—for group #4, 25 mg/kg—for group #5, 50 mg/kg—for group #6, 100 mg/kg—for group #7, 500 mg/kg—for group #8 in PBS) is administered.

In another example, the day following bleomycin treatment, Compound selected from the table 1 ab and Table 1c is administered in dosage and root of administration selected from the Example 1.

In yet another example, the day following bleomycin treatment, Compound selected from the table 1 ab and Table 1c is administered via intraperitoneal injection in the dosage 10-fold less than LD50.

In yet another example, the day following bleomycin treatment, Compound selected from the table 1 ab and Table 1c is administered via intraperitoneal injection in the dosage 100-fold less than LD50.

In yet another example, the day following bleomycin treatment, Compound selected from the table 1 ab and Table 1c is administered in dosage, regimen and root of administration selected from the Example 1.

In one example, 3MR mice is treated via intraperitoneal injection with Compound selected from the table 1 ab and Table 1c for 5 consecutive days, followed by 5 days of rest, followed by a second treatment cycle of 5 consecutive days.

In another example, 3MR mice is treated with Compound selected from the table 1 ab and Table 1c in dosage, regimen and root of administration selected from the Example 1.

Untreated mice receive an equal volume of vehicle. At 7, 14, and 21 days post-bleomycin treatment, lung function is assessed by monitoring oxygen saturation using the MouseSTAT PhysioSuite pulse oximeter (Kent Scientific), Animals are anesthetized with isoflurane (1.5%) and a toe clip is applied. Mice are monitored for 30 seconds and the average peripheral capillary oxygen saturation (Sp0₂) measurement over this duration is calculated. The bleomycin administration significantly reduce Sp0₂ levels in vehicle treated mice, and removal of senescent cells results in higher Sp0₂ levels, which approach normal levels at 21 days post bleomycin administration. At 21 days post-bleomycin treatment, airway hyper-reactivity (AHR) of mice is examined. AHR of mice is measured by methacholine challenge while other parameters of lung function (airway mechanics, lung volume and lung compliance) is determined using a SCIREQ flexiVent ventilator. While under ketamine/xylazine anesthesia and subjected to cannulation of the trachea via a tracheostomy (19Fr blunt Luer cannula), airway resistance (elastance) and compliance of mice are assessed at baseline and in response to increasing concentrations of methacholine (0 to 50 mg/ml in PBS) delivered via nebulization (AeroNeb) as described in Aravamudan et al. (Am. J. Physiol. Lung Cell. Mol. Physiol. (2012) 303: L669-L681). Animals are maintained at 37° C., and while under muscle paralysis (pancuronium); airway function is measured by using the FlexiVent™ ventilator and lung mechanics system (SCIREQ, Montreal, Quebec, Canada), which is housed on Stabile 8. In vehicle treated mice, bleomycin administration increase lung elastance, whereas Compound selected from the table 1 ab and Table 1c treatment reduces lung elastance. The bleomycin administration reduces static compliance and (dynamic) compliance in vehicle treated mice. Clearance of senescent cells with Compound selected from the table 1 ab and Table 1c in bleomycin exposed mice improves compliance values. Mice are euthanized by i.p injection of pentobarbital.

Bronchoalveolar lavage (BAL) fluids and lungs is obtained and analyzed.

Hydroxyproline content of lungs is measured as described in Christensen et al. (1999, Am. J. Pathol. 155:1773-1779), and quantitative histopathology is performed. RNA is extracted from lung tissue to measure senescence cell markers by qRT-PCR in treated and control mice.

The effect of clearance of senescence cells in the bleomycin induced lung injury model of IPF may also be studied in INK-ATT AC transgenic mice in the study design described above. INK-ATT AC (p16^{Ink4a} apoptosis through targeted activation of caspase) transgenic mice have an FK506-binding protein (FKBP)-caspase 8 (Casp8) fusion polypeptide under the control of the p16^{Ink4a} promoter (see, e.g., Baker et al, Nature, supra; Int'l Patent Application Publication No. WO/2012/177927). In the presence of AP20187, a synthetic drug that induces dimerization of a membrane bound myristoylated FKBP-Casp8 fusion protein, senescent cells specifically expressing the FKBP-Casp8 fusion protein via the p16^{Ink4a} promoter undergo programmed cell death (apoptosis) (see, e.g., Baker, Nature, supra, FIG. 1 therein). A second study also assesses the effect of clearance of senescence cells using a compound selected from the table 1 ab and Table 1c in C57BL6/J mice that have bleomycin induced lung injury. Bleomycin is administered to 6 week old C57BL6/J mice as described above. A compound selected from the table 1 ab and Table 1c is administered during the first and third week post-bleomycin treatment in the dosage and root of administration selected from the Example 1. Control mice are treated with vehicle. At 21 days post-bleomycin treatment, clearance of senescent cells and lung function/histopathology is assessed.

In a second animal model for pulmonary diseases (e.g., COPD), mice are exposed to cigarette smoke. The effect of a Compound selected from the table 1 ab and Table 1c on the mice exposed to smoke is assessed by senescent cell clearance, lung function, and histopathology.

Six week-old 3 MR (n=70) or INK-ATTAC (n=70) mice are chronically exposed to cigarette smoke generated from a Teague TE-10 system, an automatically-controlled cigarette smoking machine that produces a combination of side-stream and mainstream cigarette smoke in a chamber, which is transported to a collecting and mixing chamber where varying amounts of air is mixed with the smoke mixture. The COPD protocol is adapted from the COPD core facility at Johns Hopkins University (at Internet site web.jhu.edu/Biswal/exposure_core/copd.html #Cigarette_Smoke) (Rangasamy et al, 2004, J. Clin. Invest. 114: 1248-1259; Yao et al, 2012, J. Clin. Invest. 122:2032-2045). Mice receive a total of 6 hours of cigarette smoke exposure per day, 5 days a week for 6 months. Each lighted cigarette (3R4F research cigarettes containing 10.9 mg of total particulate matter (TPM), 9.4 mg of tar, and 0.726 mg of nicotine, and 11.9 mg carbon monoxide per cigarette [University of Kentucky, Lexington, KY]) is puffed for 2 seconds and once every minute for a total of 8 puffs, with the flow rate of 1.05 L/min, to provide a standard puff of 35 cm³. The smoke machine is adjusted to produce a mixture of side stream smoke (89%) and mainstream smoke (11%) by smoldering 2 cigarettes at one time. The smoke chamber atmosphere is monitored for total suspended particulates (80-120 mg/m³) and carbon monoxide (350 ppm).

In one example, beginning at day 7, (10) INK-ATTAC and (10) 3 MR mice are treated with compound selected from the table 1 ab and Table 1c (3× per week) (5 consecutive days of treatment followed by 16 days off drug, repeated until the end of the experiment), respectively.

In another example, beginning at day 7, (10) INK-ATTAC and (10) 3 MR mice are treated with compound selected from the table 1 ab and Table 1c administered in the dosage and root of administration selected from the Example 1 (5 consecutive days of treatment followed by 16 days off drug, repeated until the end of the experiment), respectively.

An equal number of mice received the corresponding vehicle. The remaining 30 mice (15 INK-ATTAC and 15 3 MR) were evenly split with 5 of each genetically modified strain placed into treatment groups.

One group (n=30) receives a compound selected from the table 1 ab and Table 1c in PBS, treated 14 days consecutively followed by 14 days off drug, repeated until the end of the experiment). The dosage and root of administration is selected from the Example 1.

An additional 70 animals that did not receive exposure to cigarette smoke are used as controls for the experiment.

After two months of cigarette smoke exposure, lung function is assessed by monitoring oxygen saturation using the MouseSTAT PhysioSuite pulse oximeter (Kent Scientific). Animals are anesthetized with isoflurane (1.5%) and the toe clip was applied. Mice are monitored for 30 seconds and the average peripheral capillary oxygen saturation (Sp0₂) measurement over this duration is calculated. The clearance of senescent cells via Compound selected from the table 1 ab and Table 1c results in statistically significant increase in Sp0₂ levels in mice after 2 months of cigarette smoke exposure compared to untreated controls.

At the end of the experimental period, airway hyper-reactivity (AHR) of mice to methacholine challenge using a SCIREQ flexiVent ventilator and lung mechanics system is examined as described above. After AHR measurement, mice are killed by i.p. injection of pentobarbital for in-depth analysis of lung histopathology as previously described (Rangasamy et al, 2004, J. Clin. Invest. 114:1248-1259). Briefly, lungs are inflated with 0.5% low-melting agarose at a constant pressure of 25 cm. Part of the lung tissue is collected for RNA extraction and qRT-PCR analysis of senescent markers. Other parts of lungs are fixed in 10% buffered formalin and embedded in paraffin. Sections (5 um) are stained with hematoxylin and eosin. Mean alveolar diameter, alveolar length, and mean linear intercepts are determined by computer-assisted morphometry with Image Pro Plus software (Media Cybernetics).

The potential therapeutic effect of clearance of senescent cells after COPD is fully developed may be assessed in 3MR or INK-ATTAC mice. Six week-old 3MR or INK-ATTAC mice are chronically exposed to cigarette smoke for 6 months as described above.

In some examples, at 6 months following the start of smoke exposure, 3MR or INK-ATTAC mice are treated with Compound selected from the table 1 ab and Table 1c (5 consecutive days of treatment followed by 16 days off drug) in a dosage and root of administration selected from the table 1 ab and Table 1c, respectively, until 9 months following the start of smoke exposure, when assessment of senescent cell clearance, lung function, and histopathology is performed.

In some examples, the lung function of the treatment group is improved in comparison with the control group even without the direct senolytic action of compound selected from the table 1 ab and Table 1c.

### Example 21

Effect of the Compound Selected from the Table 1 Ab and Table 1c to Reduce Chemotherapy Related Side Effects

The capability of COMPOUND SELECTED FROM THE TABLE 1 ab and Table 1c, to reduce chemotherapy related side effects, such as fatigue, is examined in p16-3MR transgenic mice. In addition to doxorubicin, paclitaxel also induces cellular senescence when administered to animals.

Paclitaxel induces senescence and SASP in p16-3MR transgenic mice. Groups of mice (n=20 in each group) are treated three times every two days with 20 mg/kg paclitaxel or vehicle. Senescence is observed as shown by luminescence in mice treated with paclitaxel (known in the art). The level of mRNA in skin is determined for each of the target genes: pi 6, 3MR transgene, and IL-6. The levels of mRNA for each of pi 6, 3MR, and IL-6 increased in paclitaxel treated animals compared with vehicle treated animals.

In this experiment, paclitaxel is administered to groups of p16-3mr mice three times, every two days.

In one example, two days after the third dose of paclitaxel, COMPOUND SELECTED FROM THE TABLE 1 ab and Table 1c is administered daily for three days (days 1, 2, and 3) at different dosages (0.01 mg/kg—for group #1, 0.1 mg/kg—for group #2, 1 mg/kg—for group #3, 10 mg/kg—for group #4, 25 mg/kg—for group #5, 50 mg/kg—for group #6, 100 mg/kg—for group #7, 500 mg/kg—for group #8)

In another example, COMPOUND SELECTED FROM THE TABLE 1 ab and Table 1c is administered intraperitoneally daily for seven days after paclitaxel administration at different dosages (0.01 mg/kg—for group #1, 0.1 mg/kg—for group #2, 1 mg/kg—for group #3, 10 mg/kg—for group #4, 25 mg/kg—for group #5, 50 mg/kg—for group #6, 100 mg/kg—for group #7, 500 mg/kg—for group #8).

In yet another example, COMPOUND SELECTED FROM THE TABLE 1 ab and Table 1c is administered daily for seven days after paclitaxel administration at dosages and by the root of administration selected from the Example 1.

Two days after the last dose of COMPOUND SELECTED FROM THE TABLE 1 ab and Table 1c, all groups of animals are housed in metabolic cages (promethion, sable systems international, Las Vegas, NV) to monitor voluntary exercise as determined by wheel counts. Data is collected and analyzed two days later.

Administration of COMPOUND SELECTED FROM THE TABLE 1 ab and Table 1c restores wheel count reduction caused by chemotherapy treatment.

### Example 22

Compound Selected from the Table 1 ab and Table 1c Improves Glucose Tolerance and Insulin

### Sensitivity

Groups of p16-3MR mice (n=27) are fed a high fat diet for four months mice or a regular chow diet.

In one example, Animals are then treated with COMPOUND SELECTED FROM THE TABLE 1 ab and Table 1c (3 rounds administered daily for five consecutive days at the dosage 50-fold less than LD50) or vehicle.

In another example, Animals are then treated with COMPOUND SELECTED FROM THE TABLE 1 ab and Table 1c via intraperitoneal injection (3 rounds administered daily for five consecutive days at the dosage 10-fold less than LD50) or vehicle.

In yet another example, Animals are then treated with COMPOUND SELECTED FROM THE TABLE 1 ab and Table 1c via intraperitoneal injection (3 rounds administered daily for five consecutive days at the dosage 100-fold less than LD50) of vehicle.

In yet another example, Animals are then treated with COMPOUND SELECTED FROM THE TABLE 1 ab and Table 1c (3 rounds administered daily for five consecutive days at the dosage and root of administration selected from the Example 1) or vehicle.

In yet another example, Animals are then treated with COMPOUND SELECTED FROM THE TABLE 1 ab and Table 1c according to the protocol selected from the Example 1.

A glucose bolus is given at time zero, and blood glucose is monitored at 20, 30, 60, and 120 minutes after delivering glucose to determine glucose disposal). This is also quantified as “area under the curve” (AUC), with a higher AUC value indicating glucose intolerance. AUCs of mice treated with COMPOUND SELECTED FROM THE TABLE 1 ab and Table 1c are lower than their vehicle-treated counterparts although not as low as chow-fed animals. Hemoglobin Ale is lower in COMPOUND SELECTED FROM THE TABLE 1 ab and Table 1c-treated mice suggesting that the animals longer-term glucose handling is also improved.

Insulin sensitivity was also determined (Insulin Tolerance Testing (ITT)). COMPOUND SELECTED FROM THE TABLE 1 ab and Table 1c-treated mice show a greater decrease in blood glucose at 0, 14, 30, 60, and 120 minutes after the administration of glucose bolus at time zero, suggesting that senescent cell clearance improves insulin sensitivity.

Changes in weight, body composition, and food intake are also monitored. In this example, treatment by COMPOUND SELECTED FROM THE TABLE 1 ab and Table 1c will not alter body weight, body composition monitored by measuring percent of fat, or food intake (measured in grams per week).

### Example 23

Compound Selected from the Table 1 ab and Table 1c Sustains Cardiac Stress Resistance with Aging

The cohorts of INK-ATTAC transgenic mice on FVB×129Sv/E×C57BL/6 mixed or C57BL/6 pure genetic backgrounds are established.

In one example, at 12 months age, one half of each cohort is injected three times/week with COMPOUND SELECTED FROM THE TABLE 1 ab and Table 1c 0.2 mg/kg and 2 mg/kg COMPOUND SELECTED FROM THE TABLE 1 ab and Table 1c for the mixed and the pure C57BL/6 cohorts, respectively), while the other half of each cohort received vehicle.

In another example, the day following bleomycin treatment, Compound selected from the table 1 ab and Table 1c is administered in dosage and root of administration selected from the Example 1.

In yet another example, the day following bleomycin treatment, Compound selected from the table 1 ab and Table 1c is administered via intraperitoneal injection in the dosage 10-fold less than LD50.

In yet another example, the day following bleomycin treatment, Compound selected from the table 1 ab and Table 1c is administered via intraperitoneal injection in the dosage 100-fold less than LD50.

In yet another example, the day following bleomycin treatment, Compound selected from the table 1 ab and Table 1c is administered in dosage, regimen and root of administration selected from the Example 1.

At 18 months, subsets of male and female mice from each cohort are subjected to a cardiac stress test, in which mice are injected with a lethal dose of isoproterenol (680 mg/kg) and the time to cardiac arrest was recorded.

While 18-month-old untreated (vehicle) mice consistently show a marked acceleration of cardiac arrest compared to 12-month-old control mice, COMPOUND SELECTED FROM THE TABLE 1 ab and Table 1c-treated mice sustain youthful cardio-protection against isoproterenol, regardless of gender and genetic background.

Cardio-protective signaling pathways are known to provide tolerance to metabolic stresses such as ischemia and hypoxia decline (Granfeldt et al., 2009, Cardiovasc. Res. 83:234-246). However, cardio-protective signaling deteriorates with aging, thus decreasing the functional and adaptive reserve capacity of the heart (Ogawa et al., 1992, Circulation 86:494-503; Wiebe et al., 1998, Clin. J. Sport Med. 8:272-279).

### Example 24

### Animal Studies Retinopathy

The breakdown of vascular beds in ischemic retinopathies—whether glycemia-driven, as in diabetic retinopathy (DR), or oxygen-driven, as in retinopathy of prematurity (ROP)—yields hypoxic/ischemic central nervous system (CNS) tissue subjected to biochemical and inflammatory stressors that compromise cellular function. These avascular zones are the source of proangiogenic factors that mediate pathological angiogenesis, as evidenced by the clinical success of ablation of these areas with laser photocoagulation therapy.

### Oxygen-Induced Retinopathy

Mouse pups from different strains (C57BL/6 WT, LysM-Cre, LysM-Cre/IRE1fl/fl, LysM-Cre/IRE1+/+, IRE1fl/fl, and LysM-Cre/ROSA26EYFPfl/fl) and their fostering mothers (CD1, Charles River) can be exposed to 75% O2 from P7 to P12 and returned to room air. This model serves as a proxy to human ocular neovascular diseases such as ROP and DR characterized by a late phase of destructive pathological. Upon return to room air, hypoxia-driven neovascularization develops from P14 onward

In some examples, a therapeutic inhibition of the SASP through intravitreal delivery of the compound selected from the table 1 ab and Table 1c in mice reduces destructive retinal neovascularization in vivo.

In some other examples, a intravitreal delivery of the compound selected from the table 1 ab and Table 1c in mice reduces destructive retinal neovascularization in vivo.

In some other examples, delivery of the compound selected from the table 1 ab and Table 1c by the protocol selected from the Example 1 in mice reduces destructive retinal neovascularization in vivo.

To elucidate the cellular processes triggered subsequent to vascular de-generation in ischemic retinopathies, mouse pups shall be subjected to a model of oxygen-induced retinopathy (OIR) that yields avascular neural zones similar to those observed in DR and ROP. Mouse pups are exposed to 75% oxygen from postnatal day 7 (P7) to P12 to induce vaso-obliteration and are returned to ambient air where maximal preretinal neovascularization is reached at P17. Further details on the model can be found in Oubaha et al., Sci. Transl. Med. 8, 362ra144 (2016).

In some examples, a single intravitreal injection of compound selected from the table 1 ab and Table 1c at P12 attenuates nuclear factor kB and IRE1a activation in mouse retinas subjected to OIR. This leads to a decrease in Il6, Cdkn1a, Cdkn2a, and Sema3A, as determined by qRT-PCR, and translates into a significant decrease in SA-b-gal at P14 and P17. In some embodiments, components of the VEGF signaling pathway are not affected.

In one example, Animals are treated with COMPOUND SELECTED FROM THE TABLE 1 ab and Table 1c at the dosage 50-fold less than LD50.

In another example, Animals are treated with COMPOUND SELECTED FROM THE TABLE 1 ab and Table 1c at the dosage 10-fold less than LD50.

In yet another example, Animals are treated with COMPOUND SELECTED FROM THE TABLE 1 ab and Table 1c at the dosage 100-fold less than LD50.

In yet another example, Animals are treated with COMPOUND SELECTED FROM THE TABLE 1 ab and Table 1c at the dosage selected from the Example 1.

In yet another example, Animals are treated with COMPOUND SELECTED FROM THE TABLE 1 ab and Table 1c at the dosage and root of administration selected from the Example 1.

In some examples, intravitreal injection of compound selected from the table 1 ab and Table 1c enhances vascular regeneration as assessed at P17 and suppresses pathological neovascularization.

In some other examples, administration of compound selected from the table 1 ab and Table 1c enhances vascular regeneration as assessed at P17 or/and suppresses pathological neovascularization.

These data support the potential of compound selected from the table 1 ab and Table 1c in the treatment of pathological retinal angiogenesis.

### Application in Humans

In some embodiments, Compound selected from the table 1 ab and Table 1c can be administered intravitreally and all patients will be followed for approximately 6 months.

In some embodiments, Compoud selected from the table 1 ab and Table 1c can be administered by the protocol selected from the Example 10.

In some embodiments, Inclusion Criteria:

Nonproliferative diabetic retinopathy (DR) patients with DME.

Center-involved DME with central subfield thickness (CST)≥350 μm on SD-OCT.

BCVA in the study eye (most affected) of 35 Early Treatment Diabetic Retinopathy Study (ETDRS) letters or worse of 6 months duration or longer prior to screening.

The measurements of efficacy are conducted by the methods known it the art.

In some examples, at least one of the symptoms of DR is alleviated by the treatment. Non-limiting examples of the symptoms are Spots or dark strings floating in the vision (floaters), Blurred vision, Fluctuating vision, Impaired color vision, Dark or empty areas in the vision, Vision loss.

### Example 25

### Age-Related Bone Loss

Compound selected from the table 1 ab and Table 1c prevents age-related bone loss in mice

Use of compound selected from the table 1 ab and Table 1c prevents age-related bone loss. Experimental design for testing the effect using a transgenic approach on age-related bone loss: 20-month-old female INK-ATTAC mice are randomized to either vehicle (n=13) or COMPOUND SELECTED FROM THE TABLE 1 ab and Table 1c (n=32 for each treatment group) treatment.

In one example, compound selected from the table 1 ab and Table 1c is treated (intraperitoneally [i.p] twice weekly) for 4 months at dosage 10-fold less than LD50.

In another example, compound selected from the table 1 ab and Table 1c is treated (intraperitoneally [i.p] twice weekly) for 4 months at dosage 50-fold less than LD50.

In yet another example, compound selected from the table 1 ab and Table 1c is treated (intraperitoneally [i.p] twice weekly) for 4 months at dosage 100-fold less than LD50.

In yet another example, compound selected from the table 1 ab and Table 1c is treated (intraperitoneally [i.p] twice weekly) for 4 months at dosage 500-fold less than LD50.

In yet another example, compound selected from the table 1 ab and Table 1c is treated (intraperitoneally [i.p] twice weekly) for 4 months at dosage 1000-fold less than LD50.

In yet another example, compound selected from the table 1 ab and Table 1c is treated (intraperitoneally [i.p] twice weekly) for 4 months at different dosages (0.01 mg/kg—for group #1, 0.1 mg/kg—for group #2, 1 mg/kg—for group #3, 10 mg/kg—for group #4, 25 mg/kg—for group #5, 50 mg/kg—for group #6, 100 mg/kg—for group #7, 500 mg/kg—for group #8.

In yet another example, compound selected from the table 1 ab and Table 1c is treated (intraperitoneally [i.p] twice weekly) for 4 months at dosages and by the root of administration selected from the Example 1.

In yet another example, compound selected from the table 1 ab and Table 1c is treated at dosages, regimen and by the root of administration selected from the Example 1.

The other protocol in details is described in Farr J N, Xu M, Weivoda M M, et al. Targeting cellular senescence prevents age-related bone loss in mice [published correction appears in Nat Med. 2017 Nov. 7; 23 (11): 1384]. Nat Med. 2017; 23(9): 1072-1079. doi:10.1038/nm.4385

In some examples, The compound, selected from the table 1 ab and Table 1c treatment results in lower p16Ink4a mRNA expression in bone relative to vehicle-treated mice as well as lower EGFP mRNA encoded by the INK-ATTAC transgene, consistent with clearance of senescent cells. This can also be confirmed by fewer senescent osteocytes in compound selected from the table 1 ab and Table 1c treated—versus vehicle-treated mice, as assessed by an established senescence biomarker (senescence-associated distension of satellites [SADS]

In some examples, Relative to vehicle treatment, compound selected from the table 1 ab and Table 1c-treated mice has better spine trabecular bone microarchitecture, with similar improvements in trabecular bone at the femur.

In some examples, Compound selected from the table 1 ab and Table 1c treatment also results in higher cortical thickness and bone strength (by micro-finite element analysis [μFEA]) at the femur.

In some embodiments, consistent with the uFEA results, better caudal vertebrae bone biomechanical properties are found (if assessed by biomechanical testing) in compound selected from the table 1 ab and Table 1c—as compared to vehicle-treated mice, whereas bone material properties (assessed by nanoindentation testing) are not different between compound selected from the table 1 ab and Table 1c—and vehicle-treated mice.

Trabecular bone histomorphometry in the old INK-ATTAC mice demonstrates lower bone resorption (osteoclast numbers per bone perimeter) in compound selected from the table 1 ab and Table 1c-versus vehicle-treated mice, without a coupled reduction in bone formation indices (osteoblast numbers, mineral apposition rate, and bone formation rate.

COMPOUND SELECTED FROM THE TABLE 1 ab and Table 1c treatment results in lower osteoclast numbers, resulting in an improvement in the osteoblast: osteoclast ratio in the old COMPOUND SELECTED FROM THE TABLE 1 ab and Table 1c-treated mice. The lower bone resorption in COMPOUND SELECTED FROM THE TABLE 1 ab and Table 1c—versus vehicle-treated mice is also evident on the endocortical surface of the femur additionally, on this surface, osteoblast numbers, mineral apposition rate, and bone formation rate are all higher in the COMPOUND SELECTED FROM THE TABLE 1 ab and Table 1c-versus vehicle-treated mice, thus suggesting that the impairment in bone formation is improved by COMPOUND SELECTED FROM THE TABLE 1 ab and Table 1c.

### Mouse Strains and Drug Treatments

Mice are housed in ventilated cages and maintained within a pathogen-free, accredited facility under a 12-hr light/dark cycle with constant temperature (23° C.) and access to food (standard mouse diet, Lab Diet 5053, St. Louis, MO) and water ad libitum. All experiments are performed in accordance with IACUC guidelines. Both sexes are studied as specified below.

Genotype them to select INK-ATTAC heterozygotes and aged them to 6-12 (young) or 20 (old) months. All experiments using the INK-ATTAC mice are performed on transgenic line 3, which contains 13 copies of the transgene inserted into a single locus.

In some examples, Female INK-ATTAC mice from this cohort are randomly assigned to be injected (twice weekly beginning at 20 months of age for a total of 4 months (all old mice are sacrificed at 24 months of age).

In some examples, Young (12-month) female INK-ATTAC mice are treated using the same dosing regimen for 1.5 months (to be sacrificed at 13.5 months of age). Obtain plasma samples from young (6-month) female INK-ATTAC mice that are treated using the same dosing regimen for 4 months.

C57BL/6 male mice are obtained from the National Institute on Aging (NIA) at 7-, 20- or 22-months of age. Alternatively, C57BL/6 breeding colonies are maintained to generate animals for in vitro osteoclastogenesis assays.

In some examples, Twenty-month-old male C57BL/6 mice are randomly assigned to once monthly treatments by oral gavage with compound selected from the table 1 ab and Table 1c or vehicle for four months 5.

### Tissue Collection

Mice are sacrificed, body mass is recorded and serum/plasma is collected via cardiac puncture at sacrifice and stored at −80° C. The right femur/tibia and lumbar vertebrae are fixed in 10% neutral buffered formalin and stored in ethanol to be used for micro-computed tomography (μCT), histomorphometry, and SADS). The remaining vertebrae are used for osteocyte-enriched cell isolations. The tail is collected at the base and stored in PBS soaked gauze at −20° C. for biomechanical compression and biomaterial nanoindentation testing of the caudal vertebrae. For in vitro analyses of osteoclast progenitors or osteoclastogenesis, left femurs/tibiae are isolated and dissected free of soft tissue, epiphyses are removed, and marrow is flushed with PBS. As described in “Xu M, et al. JAK inhibition alleviates the cellular senescence-associated secretory phenotype and frailty in old age. Proc Natl Acad Sci USA. 2015; 112:301-310”, perigonadal adipose tissue is collected and either fixed (for SA-β-Gal staining) or snap frozen in liquid nitrogen for RNA and subsequent rt-qPCR analyses.

### Isolation of Osteocyte-Enriched Cells

Detailed methods and validation of our osteocyte-enriched cell isolation protocol are presented elsewhere. Briefly, mouse vertebrae are stripped of muscle/connective tissues, minced, and sequentially digested twice for 30-min in collagenase (Liberase; Roche Diagnostics GmbH, Mannheim, Germany). As shown previously, the remaining cell fraction represents a highly enriched population of osteocytes used for rt-qPCR analyses.

### SADS Analysis of Senescent Osteocytes

Senescence-associated distention of satellites (SADS) are measured in vivo in a blinded fashion as described in the art in osteocytes located in cortical bone in the long bone diaphyses. Briefly, the decalcified right tibiae of mice are fixed, embedded in methylmethacrylate (MMA), and sectioned, followed by fluorescent in situ hybridization (FISH). Bone sections are crosslinked with 4% paraformaldehyde (PFA) for 20 min, is hed 3 times in PBS (5 min each), and dehydrated in graded ethanol as follows: 70%, 90%, and 100% (3 min each). Following a brief air drying, sections are denatured for 10 min at 80° C. in hybridization buffer: 70% formamide (Sigma), 25 mM MgCl2, 0.1M Tris (pH 7.2), 5% blocking reagent (Roche) containing 1.0 μg/mL of Cy3-labelled (F3002, reacts to both human and mouse), and CENPB-specific (ATTCGTTGGAAACGGGA) peptide nucleic acid (PNA) FISH probe (Panagene Inc, Korea), followed by hybridization for 2 hrs at room temperature in the dark. Slides are is hed, stained, and mounted with vectashield DAPI-containing mounting media (Life Technologies). In addition, we performed the SADS assay on primary murine osteocytes isolated from long-bone diaphyses using the Bonewald Laboratory technique cells are either untreated or treated with 10 Gy of cesium irradiation (to induce senescence) and cultured in a humidified incubator (maintained at 37° C. and 5% CO2) for 20 days. SADS are visualized using confocal microscopy (Mayo Clinic Microscopy and Cell Analysis Core) as described. The number of decondensed/elongated centromeres per osteocyte are quantified, as described in the art, a cut-off for defining cell senescence is ≥4 SADS per cell

### Skeletal Phenotyping

All imaging is performed in a blinded fashion. Areal bone mineral density (aBMD; g/cm2) of the lumbar spine (L1-L4) is measured by dual-energy X-ray absorptiometry (DXA) using a Lunar PIXImus densitometer (software version 1.44.005; Lunar Corp., Madison, WI). Measures of total (metaphysis), trabecular (metaphysis), and cortical (midshaft diaphysis) volumetric BMD (vBMD; mg/cm3) at the tibia are obtained using peripheral quantitative computed tomography (pQCT; Stratec XCT Research SA Plus, software v5.40, Nordland Medical Systems, Fort Atkinson, WI). Quantitative analysis of the lumbar spine (L4-L6) and distal femoral metaphysis are performed using the Viva Scan 40 uCT scanner (Scanco Medical AG, Basserdorf, Switzerland) with the following parameters: 55 kVp, 145 mA, high resolution, 21.5 diameter, 10.5 μm voxel size, 300 ms integration time. Using two-dimensional (2D) data from scanned slices, 3D analysis is used to calculate morphometric parameters at both the lumbar spine (200 slices) and distal femoral metaphysis (100 slices) defining trabecular bone mass and microarchitecture, including trabecular bone volume fraction (BV/TV; %), trabecular number (Tb.N; 1/mm), trabecular thickness (Tb.Th; mm), trabecular separation (Tb.Sp; mm [higher values are associated with weaker bone]), and the structure model index (SMI), which indicates whether trabeculae are stronger, plate-like (lower values) or weaker, rod-like (higher values). Cortical thickness (Ct.Th; mm) is assessed at the distal femoral metaphysis (50 slices). Micro-finite element analysis (μFEA) is performed at the femoral metaphysis to assess failure load (N; i.e., bone strength) using the manufacture's software (Scanco Medical AG, Basserdorf, Switzerland; Finite Element-Software Version 1.13). All μCT parameters are derived using the manufacturer's protocols.

### Compression Loading

Loading tests are performed in a blinded fashion. The sixth caudal vertebrae (Ca6) is removed from the tail, stripped of soft tissue, and measured end-to-end with a digital caliper (ABSOLUTE, Mitutoyo, Aurora, IL). Vertebrae are soaked in PBS for 15 to 30 minutes before testing to ensure hydration. Cyanoacrylate adhesive is applied to each end of the bones to mount them between two #10-32 stainless steel nuts filled with PMMA resin. The assembly is loaded into an alignment guide to impart parallel loading surfaces at each end of the bone while the adhesive cured for five minutes. Compression testing is conducted with a servohydraulic test system instrumented with a 450-Newton (N) capacity load cell (Mini Bionix II, MTS Systems, Eden Prairie, MN). Bones are pre-loaded to 1 N and then loaded until failure under displacement control at a rate of 0.02 mm/s, as previously described. Force and displacement data are collected at a sample rate of 100 Hz. The yield load (N), ultimate load (N), ultimate displacement (mm), and energy to ultimate failure (mJ) are evaluated.

### Nanoindentation

Nanoindentation is performed in a blinded fashion. The seventh caudal vertebra (Ca7) is removed from the tail and stripped of soft tissue. Specimens are embedded in PMMA resin (Lecoset 100, Leco, St. Joseph, MO) and then sectioned transversely within the body of the vertebrae with a low-speed diamond saw (Isomet, Buehler, Lake Bluff, IL). Sections are manually polished with a polishing/griding system (Ecomet 250, Buehler) using successively finer abrasive cloths (400, 600, 800, and 1200 grit) with a final polish using a microcloth and slurry of 0.05 μm aluminum abrasive (Union Carbide, Houston, TX). Indentation testing is conducted on cortical bone with a nanoindentation system (TI 950, Hysitron, Minneapolis, MN) equipped with a diamond Berkovitch pyramidal tip. Four sites, widely distributed around cross-section, are tested on each bone. At each site, a 2×2 array is indented with 15 μm spacing between indents. Indentation is conducted under load control at a rate of 500 μN/s to a peak load of 2000 μN with a 60 s hold before unloading to reduce viscoelastic effects, as previously described26. The reduced modulus (Er; GPa), and hardness (H; GPa), are calculated using the Oliver-Pharr model, as previously described in the art. Measures are averaged over the four indents of the array to generate a value for the site. Values obtained at the four sites are averaged to generate an aggregate value for each specimen.

### Histomorphometric Analyses

All histomorphometric analyses are performed in a blinded fashion. For dynamic histomorphometric analyses, mice are injected subcutaneously with Alizarin Red (0.1 mL/animal, 7.5 mg/mL) and calcein (0.1 mL/animal, 2.5 mg/mL) on days 9 and 2, respectively, prior to euthanasia. The lumbar vertebrae and right femur are isolated from female INK-ATTAC mice treated with vehicle or COMPOUND SELECTED FROM THE TABLE 1 ab and Table 1c or male C57BL/6 mice treated with vehicle or D+Q and are embedded in MMA, sectioned, and stained with Masson Trichrome to assess osteoblast numbers per bone perimeter (N.Ob/B.Pm,/mm), or stained for tartrate-resistant acid phosphatase (TRAP) activity to assess osteoclast numbers per bone perimeter, N.Oc/B.Pm,/mm). Alternatively, sections are left unstained to quantify mineralizing surfaces (mineral apposition rate, MAR, mcm/d; bone formation rate per bone surface, BFR/BS, mcm3/mcm2/d). Femurs obtained from vehicle or JAKi-treated C57BL/6 male mice are decalcified for two weeks in 12.5% EDTA, followed by paraffin embedding, sectioning, and staining with Goldner's Trichrome stain. Osteoblast (N.Ob/B.Pm) and osteoclast (N.Oc/B.Pm) numbers are assessed. The determination of bone marrow adipocyte parameters has been described in the art previously. Briefly, adipocyte number, adipocyte perimeter (mm), and adipocyte volume per tissue volume are measured by tracing out individual adipocytes in all the fields analyzed. All histomorphometric measurements and calculations are performed with the Osteomeasure Analysis system (Osteometrics, Atlanta, Georgia).

### Osteoclast Differentiation and Quantification

Following euthanasia, long bones are isolated from C57BL/6 mice, 5-8 weeks of age. As described above, bone marrow is flushed with sterile PBS. Red blood cells (RBCs) are lysed using RBC lysis buffer (1×) (eBioscience, San Diego, CA), and marrow counts are obtained. Equal cell numbers are cultured overnight in vehicle (negative control), 25 ng/mL rmM-CSF (positive control) (R&D Systems, Minneapolis, MN), control CM, senescent CM, or CM from senescent cells treated with the JAK inhibitor 1 (SEN/JAKi). CM treatments are 1:1 with a-Minimal Essential Medium (MEM, Invitrogen, Carlsbad, CA) with 10% fetal bovine serum (FBS) and 1× antibiotic/antimycotic. Negative and positive controls are 1:1 with control RPMI 1640 used for AdMSC culture. As a control for the presence of the JAK inhibitor 1, cells are treated with control CM or senescent CM with the addition of freshly added vehicle (DMSO) or JAK inhibitor 1 (0.3 μM final concentration). The non-adherent populations are collected 24 hrs after plating. Cell counts are obtained, and viability is assessed with Trypan Blue. Equal cell numbers are then plated in osteoclast differentiation medium [αMEM with 10% FBS, 1× antibiotic/antimycotic, 1:50 CMB 14-12 supernatant (source of M-CSF44) and 200 ng/mL mouse receptor activator of NFκB ligand (RANKL)-GST (generated using a recombinant RANKL expression construct provided by Dr. Beth Lee, Ohio State University)]. Osteoclast differentiation is carried out for two to four days, with differentiation media replaced on day three. Mature osteoclasts formed on days three and four.

Alternatively, mouse monocytes are isolated from mouse bone marrow by negative selection using the Mouse Monocyte Isolation Kit (Miltenyi Biotec, Inc., San Diego, CA) and magnetic activated cell sorting (MACS, autoMACS-Pro magnetic cell sorter, Miltenyi Biotec, Inc.). Monocytes are cultured with CON or SEN CM overnight, as described above. Non-adherent populations are collected, counted, and plated at equal cell densities in osteoclast differentiation medium.

To assess osteoclastogenesis in vehicle or ruxolitinib (JAKi) treated mice, RBC-lysed marrow is plated directly in osteoclast differentiation medium. Osteoclast differentiation medium is replaced on day three and osteoclast cultures are fixed on day four.

Mature osteoclast cultures are fixed in 1% PFA and osteoclast differentiation is assessed by TRAP activity (Acid Phosphatase, Leukocyte Kit, Sigma, St. Louis, MO) 45. Osteoclasts are defined as TRAP positive cells with greater than three nuclei.

### MethoCult Assay

Bone marrow is plated in MethoCult™ GF 3434 Media (StemCell Technologies, Vancouver, BC, Canada). Each mouse is tested in duplicate. Cultures are incubated for 10 days and colonies are identified as monocyte/macrophage (M) or granulocyte-monocyte/macrophage (GM). Colony-forming units (CFU)-M and CFU-GM per plate are counted and averaged per mouse. Analyses are done in a blinded fashion.

### Serum/Plasma Assays

Peripheral blood for serum/plasma measurements is collected from overnight fasted mice by cardiac puncture; samples are stored at −80° C. in aliquots. Circulating plasma C-terminal telopeptide of type I collagen (CTx) levels in young and old INK-ATTAC mice are measured by enzyme immunoassay (EIA) (Immunodiagnostic Systems). Serum IL-6 and CXCL1 (IL-8) levels in JAKi-treated mice are previously measured7 using Luminex xMAP technology. Mouse ELISA kits (R&D Systems) are used to measure circulating plasma levels of IL-6, CXCL1 (mouse homolog to IL-8), and PAI-1 in all the other mice. All assays are performed in a blinded fashion.

### Statistical Analyses

Sample sizes are based on pilot or previously conducted and published experiments (e.g., Syed et al.) in which statistically significant differences are observed on bone with various interventions in other laboratories. For each experiment, replicates are done. All samples presented represent biological replicates. No samples are excluded from analyses. Investigators are blinded to allocation during experiments and outcome assessments, as noted specifically above. Data are checked for normality using histograms and all variables are tested for skewness and kurtosis. Analyses of differences between groups are performed by independent samples t-test, Wilcoxon rank-sum tests, one-way ANOVA, or repeated-measures ANOVA where justified as appropriate (see Figure Legends). Following ANOVA, the Bonferroni post-hoc test is used to adjust for multiple comparisons. Data are presented as Means±SEM (unless otherwise specified) with P<0.05 (two-tailed) considered statistically significant. Statistical analyses are performed using the Statistical Package for the Social Sciences for Windows, Version 22.0 (SPSS, Chicago, IL).

### Example 26

### Cardiovascular Dysfunction

Effects of Compound Selected from the Table 1 ab and Table 1c on Cardiovascular Function in Old Mice

Cellular senescence is associated with cardiovascular dysfunction in humans, a major cause of morbidity and mortality in the elderly. While only mild cardiac dysfunction has been reported in old mice, substantial impairment in vascular reactivity is observed in aged mice.

In one example, compound selected from the table 1 ab and Table 1c, is administered as a single dose, in a 5 days after an assaying cardiac function is made.

In another example, compound selected from the table 1 ab and Table 1c, is administered by the protocol selected from the table 1 ab and Table 1c.

The treatment of 24-month-old mice with the compound selected from the table 1 ab and Table 1c improves left ventricular ejection fraction and fractional shortening, in some embodiments, effects that are mediated by reductions in end-systolic cardiac dimensions but not cardiac preload or alterations in cardiac mass.

In some embodiments, compound selected from the table 1 ab and Table 1c yields physiologically important and consistent improvements in vascular smooth muscle sensitivity to nitroprusside.

In some embodiments, compound selected from the table 1 ab and Table 1c improves cardiovascular function and reduces morbidity and mortality from cardiovascular disease.

### Example 27

### NAFLD

The incidence of non-alcoholic fatty liver disease (NAFLD) increases with age. Cellular senescence refers to a state of irreversible cell-cycle arrest combined with the secretion of proinflammatory cytokines and mitochondrial dysfunction. Senescent cells contribute to age-related tissue degeneration.

Any one of the Compounds selected from the table 1 ab and Table 1c reduces overall hepatic steatosis in ageing, obese and diabetic mice.

In some examples, INK-ATTAC mice can be used at 24 months of age, treat them with compound selected from the table 1 ab and Table 1c in dosage selected from the Example 1 for 3 months

In some examples, INK-ATTAC mice can be used at 24 months of age, treat them with compound selected from the table 1 ab and Table 1c by protocol selected from the Example 1

In some examples, compound selected from the table 1 ab and Table 1c is administered to 24-month-old mice by intraperitoneal injection every 3 days, for 3 months.

In some examples, vehicle or Compound selected from the table 1 ab and Table 1c are administered by oral gavage once per month for 3 months.

In some examples, four treatments by Compound selected from the table 1 ab and Table 1c or vehicle (60% Phosal, 10% ethanol and 30% PEG-400) are administered for 5 consecutive days biweekly via oral gavage.).

In some examples, Mice are injected intraperitoneally with compound selected from the table 1 ab and Table 1c or vehicle for 3 days every 2 weeks for 10 weeks in a dosage selected from the Example 1.

In some examples, All mice are killed at the age of 15 months (6 male HF (3 vehicle, 3 mice treated by the compound selected from the table 1 ab and Table 1c), 9 female HF (4 vehicle, 5 mice treated by the compound selected from the table 1 ab and Table 1c), 8 male control (4 vehicle, 4 mice treated by the compound selected from the table 1 ab and Table 1c) and 5 female control (2 vehicle, 3 mice treated by the compound selected from the table 1 ab and Table 1c)).

In some examples, the animals are killed at 27 months of age.

In some examples, The treatments are not significantly alter body or liver weight.

The untreated INK-ATTAC mice at 27 months of age display higher frequencies of TAF-positive and karyomegalic hepatocytes than control AL mice at 15 months of age.

The compound selected from the table 1 ab and Table 1c treatment reduces the frequencies of TAF-positive hepatocytes and the average numbers of TAF per hepatocyte.

In some embodiments, the total DNA damage is also reduced changed with compound selected from the table 1 ab and Table 1c.

The compound selected from the table 1 ab and Table 1c also reduces the percentage of karyomegalic hepatocytes.

The compound selected from the table 1 ab and Table 1c administration results in a reduction in hepatic fat deposition in comparison with the control.

### Animals

### Mice to be Fed Ad Libitum (AL) and Dietary Restricted (DR)

Experiments are performed in male C57Bl/6 mice aged 3, 12 and 14.2±1.2 months¹⁵ purchased from Harlan (Blackthorn, UK). Mice are housed in same-sex cages in groups of 4-6 (56 ×38×18 cm³, North Kent Plastics, Kent, UK) and individually identified by an ear notch. Mice are housed at 20±2° C. under a 12 h light/12 h dark photoperiod with lights on at 0700 hours. The diet used is standard rodent pelleted chow (CRM (P); Special Diets Services, Witham, UK) for AL-fed mice and the same diet, but as smaller pellets, is offered to DR mice. The smaller pellet size reduced competition for food. DR mice are offered 60% of AL intake (calculated based on average food intake in 90 control AL mice between 5 and 12 months of age) as one ration at 0930 hours daily. Half of the animals are subjected to DR, while the other half, matched for body mass, food intake and age, served as AL controls. Additionally, control, young mice are killed at 3 months of age. DR is introduced at 3 months of age and lasted for 9-12 months. At the age of 12 months, some mice from the AL and DR groups had their dietary regime changed AL to DR or DR to AL for 3 months. All mice are killed at the time points mentioned above and at the end of the experiment.

A variety of tissues are collected. Tissues are frozen in optimal cutting temperature compound or OCT media for cryosections, snap-frozen in liquid nitrogen for biochemistry and fixed in 10% formalin for 24 h before processing and paraffin embedding. Cryosectioning is performed at 10 um intervals and paraffin-embedded tissues are cut at 3 um intervals. Haematoxylin-eosin (H&E)-stained mouse liver sections are graded for steatosis by a single expert liver pathologist (DT) who is not aware of the genotype/treatment.

Alb-Xpg transgenic mice, with a liver-specific Xpg gene inactivation, are generated and genotyped as previously described. Xpg^{f/-} Alb-Cre⁺ mice (in a C57BL6J/FVB F1 hybrid background; referred to as Alb-Cre) are heterozygous for Xpg in their entire body, except for the hepatocytes in the liver, which are homozygous for Xpg after Cre excision of the floxed allele. Littermates, with and without Cre-recombinase expression (Xpg^{f/+} Alb-Cre⁺ and Xpgf-Alb-Cre⁻ respectively), are used as controls (referred to as wt). Mice are maintained in a controlled environment (20-22° C., 12 h light; 12 h dark cycle) and are housed in individual ventilated cages under specific pathogen-free conditions. All animals has AL access to water and standard mouse food (CRM pellets, SDS BP Nutrition Ltd.; gross energy content 4.39 kcal g⁻¹ dry mass, digestible energy 3.2 kcal g⁻¹). At 6 (control: 6 male, Xpg: 6 male, 1 female) and 12 months (control: 4 male, 2 female and Xpg: 3 male, 3 female) of age, mice are killed for tissue collection. Tissues are snap-frozen in liquid nitrogen, embedded in TissueTek and sliced in 10 μm thick cryosections or fixed overnight in 10% phosphate-buffered formalin, paraffin-embedded, sectioned at 3 μm and mounted on Superfrost Plus glass slides. Oil Red O and H&E images are generated using the NanoZoomer Digital slide scanner with the NDP view software (Hamamatsu Photonics, Japan).

A new stock of INK-ATTAC transgenic mice is generated and genotyped as previously described· Mice are house at 2-5 mice per cage in a 12 h light/12 h dark cycle at 24° C. with free access to food (standard mouse diet, Lab Diet 5053, St Louis, MO, USA) and water in a pathogen-free facility.

For dietary intervention studies, INK-ATTAC mice are housed 2-5 per cage, at 22=0.5° C. on a 12-12-h day-night cycle and provided food and water AL. Mice are randomly assigned into the chow diet or HF diet group. HF food is purchased from Research Diets (cat no #D12492, 60% of calories in this diet are from fat).

Db/db mice homozygotic males and females are purchased from Jackson Laboratory (Bar Harbor, ME, stock number: 000642). Mixed gender cohort consisting of 13 male db/db, 10 female db/db, 8 male db/+ and 8 female db/+ is first time treated at the age of 4 months. Animals are killed at the age of 6 months.

Mouse adult fibroblasts Ear clippings are transported and stored (not longer than 1 h) in DMEM on ice. Punches are washed three times with serum-free media, finely cut and incubated for 2-3 h at 37° C. in 2 mg ml⁻¹ collagenase A in DMEM. A single-cell suspension is obtained by repeated pipetting and passing through a 24-G fine needle. Cells are centrifuged for 10 min at 1,000 r.p.m. and cultured in Advanced D-MEM/F-12 (DMEM, Invitrogen) plus 10% FCS (Sigma) in 3% O₂ 5% CO₂. Each cell strain is derived from a separate donor. MAFs are seeded and allowed to grow for 24 h and then X-ray irradiated with 5 or 10 Gy using a PXI X-Rad 225 (RPS Services Ltd) to induce cellular senescence. Alternatively, MAFs are treated with 100 nM of complex I inhibitor rotenone, which is replaced daily. Following 10 days of treatment, induction of senescent markers is observed.

Hepatocytes Hepatocytes are isolated from the livers of wild-type mice by digestion with collagenase from Clostridium histolyticum (Sigma) and then filtered through a 70-μm cell strainer. Cells are collected by centrifugation (500 r.p.m. for 3 min), washed three times in Krebs-Ringer buffer (Sigma) and re-suspended in Williams medium E with 10% serum (WME Gibco) and plated onto collagen-coated plates (type I collagen, BD Biosciences). After 4 h, medium is removed and cells are cultured in fresh 10% or 0.5% Williams medium E. Hepatocytes are incubated at 37° C. and 3% oxygen overnight and are exposed the next day to 10 Gy irradiation in order to induce senescence. Following 10 Gy X-ray irradiation, hepatocytes acquire a morphology characteristic of senescence and SA-β-Gal activity after 6 days. Monitoring cell numbers revealed that a small percentage of hepatocytes experienced cell death after irradiation; however, most of the cells survived and acquired a senescent-like phenotype. Non-irradiated controls are analysed 1-2 days following isolation, at the same time as irradiation took place for the irradiated cells (this is necessary to prevent overgrowth of other cell types, which are present in very low numbers).

Oil Red O Preparation of Oil Red O (Sigma-Aldrich, #01391) working solution and staining of slides is performed according to Mehlem et al.⁵⁰ and the manufacturer's instructions. Briefly, Oil Red O working solution is prepared from stock solution mixed 3:2 with water and incubated at 4° C. for 10 min. Solution is filtered through 0.45-μm filters and applied on OCT-embedded liver sections for 5 min. Slides are washed twice in water, 15 min each ish, and mounted in vectashield mounting media. For representative images, sections are counterstained with haematoxylin. Samples are imaged within 6 h. Surface of lipid droplets is quantified using the ImageJ software by measuring area occupied by red pixels.

Nile red In all, 2 μl of Nile red solution (Nile red (Sigma N3013) 150 μg ml⁻¹ in acetone) are added to 1 ml 80% glycerol. Frozen OCT-embedded liver 10-μm sections are air dried for 30 min. MAFs are washed briefly with PBS and fixed for 10 min with 2% paraformaldehyde dissolved in PBS. DAPI solution is added for 10 min and afterwards sections are washed with PBS for 5 min. Some sections are stained with ActinGreen 488 (ThermoFisher, 1 drop in 0.5 ml PBS) for 30 min and washed with PBS for 3×5 min. In all, 20-30 μl of Nile red/glycerol are directly added to each section, mounted on a glass microscope slide and covered with a cover slip. Images are taken immediately after mounting.

BODIPY 493/503 staining MAFs are washed briefly with PBS and fixed for 10 min with 2% paraformaldehyde dissolved in PBS. Cells are permeabilized with PBG for 30 min and incubated for 10 min with 4 ml ml⁻¹ of BODIPY. Cells are washed with PBS for 3×5 min, stained with DAPI solution and mounted.

Histochemistry and immunofluorescence (IF) Paraffin sections are deparaffinized with Histoclear and ethanol, and antigen is retrieved by incubation in 0.01 M citrate buffer (pH 6.0) at 95° C. for 10 min. Slides are incubated in 0.9% H₂O₂ for 30 min and afterwards placed in blocking buffer (normal goat serum 1:60 in PBS/BSA, #S-1000; Vector Laboratories) for 30-60 min at room temperature (RT). Livers are further blocked with Avidin/Biotin (Vector Laboratories, no. SP-2001) for 15 min each. MAFs are washed briefly with PBS and fixed for 10 min with 2% paraformaldehyde dissolved in PBS. Cells are permeabilized for 45 min with PBG (0.5% BSA, 0.2% Fish Gelatine, 0.5% Triton X-100 in PBS). Primary antibodies are applied overnight at 4° C. Slides are washed three times with PBS and incubated for 30 min with secondary antibody (no. PK-6101; Vector Lab). Antibodies are detected using a rabbit peroxidase ABC Kit (no. PK-6101; Vector Lab) according to the manufacturer's instructions. Substrate is developed using NovaRed (no. SK-4800; Vector Lab) or 3′3′-diaminobenzidine (no. SK4100, Vector Lab). Sections are counterstained with haematoxylin. For IF, sections are treated as before, and after the secondary antibody incubation, Fluorescein Avidin DCS (1:500 in PBS, no. A-2011, Vector Lab) is applied for 20 min. For IF on MAFs, Alexa Fluor secondary antibody (1:2,000; Molecular Probes) is applied for 30 min at RT. Sections or cells are stained with DAPI for 5-10 min and mounted in vectashield mounting media.

p21 immunohistochemistry is performed on formalin-fixed sections using rat anti-p21 antibody (clone HUGO 291H, Abcam, UK) and the ImPRESS Rat immunodetection system (MP-7444, Vector laboratories, Country) using 3′3′-diaminobenzidine (Dako, UK) as chromagen followed by counterstaining with haematoxylin. Sections are then dehydrated and coverslipped. Ten blinded consecutive non-overlapping fields are acquired at ×200 magnification and quantified as previously described

More details on the protocol can be found in Ogrodnik M, Miwa S, Tchkonia T, et al. Cellular senescence drives age-dependent hepatic steatosis. Nat Commun. 2017; 8:15691. Published 2017 Jun. 13. doi: 10.1038/ncomms15691

### Example 28

### Glaucoma

Glaucoma is comprised of progressive optic neuropathies characterized by degeneration of retinal ganglion cells (RGC) and resulting changes in the optic nerve.

Removal of endogenous senescent retinal cells after elevated intraocular pressure (IOP) elevation by a treatment with the compound selected from the table 1 ab and Table 1c prevents loss of retinal functions and cellular structure.

Compound selected from the table 1 ab and Table 1c protects retina degeneration.

In one of the examples, after unilateral IOP elevation, mice are daily injected with Compound selected from the table 1 ab and Table 1c (5 mg/kg) intraperitoneally.

In other examples, after unilateral IOP elevation, mice are treated with compound selected from the table 1 ab and Table 1c by protocol selected from the Example 1.

In some examples, At day 5, Visual evoked potential (VEP) is measured and tissue is collected for further experiments. Immunohistochemistry of Brn3a and activated caspase show increase of apoptosis at day 3 after IOP treatment. Retina flat-mount immunohistochemistry at day 5 with anti-Brn3a antibody specifically labeling ˜80% of RGC cells. Quantification of RGC number or VEP responses at day 5 (four conditions) or day 12 (additional 7 days of “recovery,” two conditions) after the 5 days treatment of p16-3MR animals with Compound selected from the table 1 ab and Table 1c. N>4 animals in each group. Statistical tests are performed using ANOVA with post hoc Tukey correction for multiple testing. *p<0.05, *p<0.001, n.s. —not significant.

Model. Upon elevated IOP damaged cells become senescent and start to express SASP molecules. While disease progresses, the SASP molecule induces senescence or apoptosis in neighbouring cells.

When senescent cells are removed using compound selected from the table 1 ab and Table 1c the neighbouring cells are not exposed to detrimental SASPs and the disease progression is significantly slowed down. Remaining cells are healthy.

In this model it can be explored whether Compound selected from the table 1 ab and Table 1c, could have a beneficial effect similar to GCV in p16-3MR mice. To this end, p16-3MR mice are treated with Compound selected from the table 1 ab and Table 1c or vehicle for 5 days by intraperitoneal injection, similarly to the experimental procedure used for GCV in doi:10.1111/acel.13089.

Performing this experiment in the transgenic mice allows direct comparison of the efficiencies of both treatments in the same mouse strain. At day five after IOP elevation, VEP measurement is performed and retinas are immunostained to quantify RGC loss. Compound selected from the table 1 ab and Table 1c treatment prevents the loss of RGC similar to what is observed in GCV-treated animals VEP analysis reveals that Compound selected from the table 1 ab and Table 1c treatment successfully prevented vision loss upon IOP elevation.

To explore whether the protective impact of the drug is caused by the sustained inhibition of the cellular processes and whether it is maintained even after the drug is no longer present, in some examples, p16-3MR mice are treated with Compound selected from the table 1 ab and Table 1c or vehicle for 5 days by intraperitoneal injection, similarly to the experimental procedure used for GCV.

In some other examples, p16-3MR mice are treated with Compound selected from the table 1 ab and Table 1c or vehicle by the protocol selected from the Example 1.

After that, the mice are no longer treated with drug or PBS and at day twelve after IOP elevation, functional measurement is performed and RGCs are quantified.

Also in this treatment regime, Compound selected from the table 1 ab and Table 1c prevents the loss of similar to what is observed in GCV-treated animals. Additionally, VEP analysis reveals that Compound selected from the table 1 ab and Table 1c treatment with seven days “chase” still successfully prevents vision loss upon IOP elevation.

### Animals

Adult p16-3MR (Demaria et al., 2014) or C57BL/6 mice (12-16 weeks old, Jackson Labs) are housed in 20° C. environment with standard (12 hr light/dark) cycling, food, and water available ad libitum. For all experiments, an equal number of male and female mice are used.

### Drug Treatment

The p16-3MR transgenic model, in which the mice carry a trimodal reporter protein (3MR) under the control of p16 regulatory region (Demaria et al., 2014), allows potent selective removal of senescent cells. The 3MR transgene encodes a fusion protein consisting of Renilla luciferase, a monomeric red fluorescent protein (mRFP) and herpes simplex virus thymidine kinase (HSV-TK) which converts ganciclovir (GCV) into a toxic DNA chain terminator to selectively kill HSV-TK expressing cells. The experimental group of animals is treated by intraperitoneal (IP) administration of GCV (Sigma, 25 mg/kg once a day) or Compound selected from the table 1 ab and Table 1c after IOP elevation, and a control group of mice is sham-treated with PBS or vehicle (DMSO). Each mouse undergo unilateral hydrostatic pressure-induced IOP elevation to 90 mm Hg, with the contralateral eye left as an untreated control. In some examples, The mice are IP injected intraperitoneally with GCV or Compound selected from the table 1 ab and Table 1c at day 0 (IOP elevation day) and continued for four consecutive days. At day 5, animals are euthanized, and retinas are isolated and immunostained with anti-Brn3a antibody to evaluate the number of RGCs. To ensure a sterile environment, compounds are prepared under the tissue culture hood using sterile PBS. The final solution is filtered through a 0.22-μm PES membrane just before injection. Tips, tubes, and syringes are sterile.

### Hydrostatic Intraocular Pressure (IOP) Elevation

Animals are anesthetized with an intraperitoneal injection of ketamine/xylazine cocktail, (100 and mg/kg, respectively), their eyes anesthetized with one drop of proparacaine (0.5%, Bausch-Lomb) and dilated with one drop of tropicamide (1%, Alcon Laboratories). Unilateral elevation of IOP is achieved by infusing balanced salt solution (Alcon Laboratories) into the anterior chamber of the eye through using an intravenous (IV) infusion set. The level of IOP increase is determined by the height of the saline bottles on the IV infusion set. Stable elevated IOP of 85-90 mm Hg is maintained for 60 min and controlled by IOP measurements using a veterinary rebound tonometer (Tonovet). Both eyes are lubricated throughout testing with an ophthalmic lubricant gel (GenTeal, Alcon Laboratories). Animals recovered on a Deltaphase isothermal pad (Braintree Scientific) until awake. The contralateral eye without IOP elevation served as a healthy non-IOP control (CTRL).

### Visual Evoked Potential

VEP measurements are taken at five days post-IOP elevation. This protocol is adapted from prior studies (Ridder & Nusinowitz, 2006). Mice are dark-adapted for at least 12 hr before the procedure. Animals are anesthetized with ketamine/xylazine and their eyes dilated as above. The top of the mouse's head is cleaned with an antiseptic solution. A scalpel is used to incise the scalp skin, and a metal electrode is inserted into the primary visual cortex through the skull, 0.8 mm deep from the cranial surface, 2.3 mm lateral to the lambda. A platinum subdermal needle (Grass Telefactor) is inserted through the animal's mouth as a reference and through the tail as ground. The measurements commenced when the baseline waveform became stable, 10-15 s after attaching the electrodes. Flashes of light at 2 log cd·s/m² are delivered through a full-field Ganzfeld bowl at 2 Hz. Signal is amplified, digitally processed by the software (Veris Instruments), then exported, and peak-to-peak responses are analyzed in Excel (Microsoft). To isolate VEP of the measured eye from the crossed signal originating in the contralateral eye, a black aluminum foil eyepatch is placed over the eye not undergoing measurement. For each eye, peak-to-peak response amplitude of the major component P1-N1 in IOP eyes is compared to that of their contralateral non-IOP controls. Following the readings, the animals are euthanized, and their eyes collected and processed for immunohistological analysis.

### Immunohistochemistry

Following euthanasia, eyes are enucleated and fixed in 4% paraformaldehyde (PFA) in PBS (Affymetrix) for 1 hr and subsequently transferred to PBS. The eyes are then dissected, the retinas flat-mounted on microscope slides, and immunostained using a standard sandwich assay with anti-Brn3a antibodies (Millipore, MAB1595) and secondary AlexaFluor 555 anti-mouse (Invitrogen, A32727). Mounted samples (Fluoromount, Southern Biotech 0100-01) are imaged in the fluorescent microscope at 20× magnification (Biorevo BZ-X700, Keyence), focusing on the central retina surrounding the optic nerve. Overall damage and retina morphology are also taken into consideration for optimal assessment of the retina integrity. Micrographs are quantified using manufacturer software for Brn3a-positive cells in 6 independent 350×350 μm areas per flat mount.

### Real-Time PCR

Total RNA extraction from mouse tissues, cDNA synthesis, and RT-qPCR experiments are performed as previously described (Skowronska-Krawczyk et al., 2015). Assays are performed in triplicate. Relative mRNA levels are calculated by normalizing results using GAPDH. The primers used for RT-qPCR are the same as in (Skowronska-Krawczyk et al., 2015). The differences in quantitative PCR data are analyzed with an independent two-sample t test.

### SA-βgal Assay to Test Senescence on Retinas Mouse Eyes

Senescence assays are performed using the Senescence b-Galactosidase Staining Kit (Cell Signaling) according to the manufacturer's protocol. Images are acquired using a Hamamatsu Nanozoomer 2.0HT Slide Scanner and quantified in independent images of 0.1 mm² covering the areas of interest using Keyence software.

### RNA-Seq Analysis

High-quality RNA is extracted using TRIzol Reagent (Invitrogen) and treated with TURBO DNA-free Kit (Invitrogen). RNA-Seq libraries are made from 1 μg total RNA per tissue sample using TruSeq stranded mRNA Library Prep Kit (Illumina, kit cat. no. 20020597) according to the manufacturer's instructions. The libraries are size-selected using Agencourt Ampure XP beads (Beckman Coulter) and quality-checked by Bioanalyzer (Agilent). The strand-specific RNA-Seq paired-end reads sequence data (PE50: 2×50 bp) are obtained on a HiSeq4000. RNA-Seq reads are counted using HOMER software considering only exonic regions for RefSeq genes.

More details on the protocol can be found in Rocha L R, Nguyen Huu V A, Palomino La Torre C, et al. Early removal of senescent cells protects retinal ganglion cells loss in experimental ocular hypertension. Aging Cell. 2020; 19 (2): e13089. doi: 10.1111/acel.13089

### Example 29

Wild-type and PS19 mice are treated with a repeating schedule of COMPOUND SELECTED FROM THE TABLE 1 ab and Table 1c, beginning at weaning age and continuing until the mice reached six months of age. Notably, this treatment prevents the upregulation of senescence-associated genes and attenuates tau phosphorylation in PS19 mice.

In some examples, mice are treated with compound selected from the table 1 ab and Table 1c by protocol selected from the Example 1.

MAPT^{P301S}PS19 mouse model of tau-dependent neurodegenerative disease accumulates p16^{INK4A}-positive senescent astrocytes and microglia.

In one example, clearance of these cells as they arise using compound selected from the table 1 ab and Table 1c prevents gliosis, hyperphosphorylation of both soluble and insoluble tau leading to neurofibrillary tangle deposition, and degeneration of cortical and hippocampal neurons, thus preserving cognitive function

In some examples, Pharmacological intervention with a Compound selected from the table 1 ab and Table 1c modulates tau aggregation.

Compared with control mice, vehicle-treated PS19; ATTAC mice has nearly double the number of cells containing X-Gal crystals in both the hippocampus and the cortex, whereas COMPOUND SELECTED FROM THE TABLE 1 ab and Table 1c-treated PS19; ATTAC mice has a similar incidence of X-Gal crystals as control mice.

A distinguishing characteristic of PS19 mice is the development of aggregates consisting of hyperphosphorylated tau protein by six months of age.

One can measure the levels of soluble total and phosphorylated tau (S202/T205) in addition to the level of insoluble phosphorylated tau in vehicle-treated PS19; ATTAC and COMPOUND SELECTED FROM THE TABLE 1 ab and Table 1c-treated ATTAC and PS19; ATTAC mice.

Vehicle-treated PS19; ATTAC mice displays increased levels both of soluble total and phosphorylated tau and of insoluble phosphorylated tau. Compound selected from the table 1 ab and Table 1c-treated PS19; ATTAC mice has identical levels of soluble total tau to vehicle-treated PS19; ATTAC mice, indicating that overexpression of tau from the transgene is maintained. Treatment of PS19; ATTAC mice with Compound selected from the table 1 ab and Table 1c-treated reduces the amount of phosphorylated tau in both the soluble and the insoluble fractions. Immunohistochemistry staining for phospho-tau modifications at S202/T205, T231 and S396 confirms that the clearance of senescent cells attenuates tau phosphorylation at several residues that are relevant for tau aggregation. Additionally, the staining of brain sections of eight-month-old mice from these same groups with thioflavin-S reveals that NFT deposition in the dentate gyrus—the site of neurogenesis in the hippocampus that is traditionally associated with memory formation and cognition—is reduced when senescent cells are removed.

PS19 mice show neurodegeneration by eight months of age. NFT deposition is attenuated upon treatment with Compound selected from the table 1 ab and Table 1c-treated in both the cortex and the hippocampus of PS19; ATTAC mice, one can perform assessments for degeneration in these areas. The overt brain size of vehicle-treated PS19; ATTAC mice is reduced compared to both ATTAC and Compound selected from the table 1 ab and Table 1c-treated PS19; ATTAC mice. In addition, one can observe localized neurodegeneration in the dentate gyrus of the hippocampus through Nissl staining in vehicle-treated PS19; ATTAC mice. The administration of Compound selected from the table 1 ab and Table 1c prevents thinning of the dentate gyrus and increased neuron density. Sequential coronal sectioning and NeuN staining reveals that the dentate gyrus is significantly reduced in area in vehicle-treated PS19; ATTAC mice, further demonstrating that senescent cells promote neurodegeneration in PS19 mice.

To test whether the effects observed upon administration of Compound selected from the table 1 ab and Table 1c results in improved cognitive function, one can perform novel-scent discrimination assessments to test for changes in short-term memory. Whereas Compound selected from the table 1 ab and Table 1c-treated ATTAC mice are more inquisitive towards the novel scent during the testing phase, vehicle-treated PS19; ATTAC mice are not. By contrast, Compound selected from the table 1 ab and Table 1c-treated PS19; ATTAC mice behaves nearly identically to control (Compound selected from the table 1 ab and Table 1c-treated ATTAC) mice, indicating that the elimination of senescent cells mitigates the short-term memory loss that will be observed in vehicle-treated PS19; ATTAC mice. the overall distance travelled by mice in all groups is unchanged, and similar results are obtained in novel-object discrimination tests that has the same setup but used visual cues instead of scents.

Thus, these results demonstrate that Compound selected from the table 1 ab and Table 1c treats neurodegeneration and loss of cognition in PS19 mice.

In some examples, Wild-type and PS19 mice are treated with a repeating schedule of COMPOUND SELECTED FROM THE TABLE 1 ab and Table 1c, beginning at weaning age and continuing until the mice reached six months of age.

In some examples, this treatment prevents the upregulation of senescence-associated genes and attenuated tau phosphorylation in PS19 mice.

In some examples, the continuous clearance of p16^{Ink4a}-expressing senescent cells by Compound selected from the table 1 ab and Table 1c before disease onset in a model of aggressive tauopathy has a treatment effect on various aspects of disease progression, including gliosis, NFT formation, neurodegeneration and cognitive decline.

In some examples, Senescent-cell clearance by Compound selected from the table 1 ab and Table 1c also has a notable effect on the accumulation of phosphorylated tau protein in both the soluble and insoluble fractions. The amount of total soluble tau is unchanged in Compound selected from the table 1 ab and Table 1c-treated PS19; ATTAC mice, indicating that the aberrant hyperphosphorylation of tau protein and subsequent aggregation into NFTs is mediated by extracellular signalling from p16^{Ink4a}-expressing senescent glial cells. In some examples, The absence of neurodegeneration in mice treated with Compound selected from the table 1 ab and Table 1c-treated demonstrates that the attenuation of disease severity does not result from the clearance of neurons containing NFTs.

In some examples, COMPOUND SELECTED FROM THE TABLE 1 ab and Table 1c can modulate senescent cells and attenuate tau phosphorylation. a, RT-qPCR analysis of the expression of senescence markers from the hippocampus (left) and the cortex (right) of six-month-old mice treated with either vehicle (−COMPOUND SELECTED FROM THE TABLE 1 ab and Table 1c) or COMPOUND SELECTED FROM THE TABLE 1 ab and Table 1c (+COMPOUND SELECTED FROM THE TABLE 1 ab and Table 1c). normalized to the WT-COMPOUND SELECTED FROM THE TABLE 1 ab and Table 1c group

Mouse strains and drug treatment. MAPT^{P301S}PS19 (PS19) mice are purchased from The Jackson Laboratory (stock no. 008169) and bred to C57BL/6 for three generations. C57BL/6 ATTAC transgenic mice are as previously described. Male PS19 mice are bred to ATTAC females to generate cohorts of ATTAC and PS19; ATTAC mice. All mice are on a pure C57BL/6 genetic background. Mice from this cohort are randomly assigned to receive COMPOUND SELECTED FROM THE TABLE 1 ab and Table 1c (AP; B/B homodimerizer; Clontech) or vehicle twice a week beginning at weaning age (3 weeks). Six-month-old short-term In some examples, Compound selected from the table 1 ab and Table 1c pulse-treated mice received a dose for five consecutive days before tissue collection. In some examples, The intervention by Compound selected from the table 1 ab and Table 1c is performed in C57BL/6 wild-type and PS19 mice. At weaning, mice are assigned to receive either COMPOUND SELECTED FROM THE TABLE 1 ab and Table 1c or vehicle (Phosal 50 PG, Lipoid NC0130871, 60%; PEG400, Sigma 91893, 30%; ethanol, 10%). COMPOUND SELECTED FROM THE TABLE 1 ab and Table 1c is administered on a repeating regimen of five consecutive days of treatment followed by 16 days of rest.

Mice are housed in a 12 h: 12 h light: dark cycle environment in pathogen-free barrier conditions as previously described in detail. Compliance with relevant ethical regulations and all animal procedures are reviewed and approved by the Mayo Clinic Institutional Animal Care and Use Committee.

Statistical analysis. Prism software is used for all statistical analysis. A Student's two-tailed unpaired t-test with Welch's correction is used in 1 ab and Table 1c and Extended Data 4b-e; two-way ANOVA with Tukey's multiple comparisons test is used for 3d and Extended Data 10; and one-way ANOVA with Tukey's multiple comparisons test is used in all other figures. For consistency in these comparisons, the following denotes significance in all figures: *P<0.05, **P<0.01, ***P<0.001. note that no power calculations are used. Sample sizes are based on previously published experiments in which differences are observed. No samples are excluded. Investigators are blinded to allocation during experiments and outcome assessment, except for rare instances in which blinding is not possible.

Senescence-associated β-galactosidase transmission electron microscopy (Gal-TEM). Detection of X-Gal crystals by TEM after senescence-associated β-galactosidase (SA-β-Gal) staining is performed as previously described, with the following alterations to accommodate central nervous tissue. Mice are transcardially perfused with ice-cold Dulbecco's phosphate-buffered saline (DPBS; pH 7.4) until fluid run-off is clear. This is followed by perfusion with 4% para-formaldehyde (PFA) for 10 min at a rate of 3 ml min 1, and then ice-cold DPBS is perfused again for 2 min at the same rate to remove the remaining fixative. Brains are then isolated and the hippocampus and cortex are dissected out. A 1 mm×1 mm piece from the CA1 and M1 regions, respectively, is then incubated in SA-β-Gal staining solution (Cell Signaling) at 37° C. for 6 h (hippocampus) or 18 h (cortex). The samples are placed in Trump's fixative overnight at 4° C. before being processed for routine transmission electron microscopy (dehydration by xylene-alcohol series, osmium tetroxide staining, and Epon resin embedding). Images are acquired and quantified using a Jeol 1400+ electron microscope with an 80-kV acceleration voltage. Two grids from each tissue are produced, and >100 cells are scanned per grid at a magnification of 20,000× to detect cells containing X-Gal crystals. On average, half of all cells examined are neurons. Cells with one or more crystals, and the total number of cells, are counted. Cells containing crystals are imaged and independently assessed for distinguishing morphology. To define cell type, the following criteria are applied. Astrocytes: circular nucleus with spattered electron density pattern; microglia: abnormally shaped nucleus with a much darker, often phagosome-containing cytoplasm; and neuron: large circular nucleus with less electron density and periodically denoted by an offshooting axon. Only cells with morphology consistent with astrocytes or microglia are clearly positive for X-gal crystals. Novel-object recognition. Novel-object recognition testing is performed as previously described. In brief, mice from each cohort are acclimatized to a 50 cm×50 cm testing environment for a period of two minutes. After acclimatization, the mice are removed, the testing area is cleaned with 70% ethanol, and two identical scented candles are placed in either corner of the testing area approximately 5 cm from either wall. Mice are reintroduced, and the ratio of both the number of visits and the time spent at each candle is recorded for a period of ten minutes. Recording is performed from above (Panasonic WV-CP294) and all video files are analysed with TopScan Version 3.00 (Clever Sys). Afterwards, the mice are removed, the testing area cleaned with 70% ethanol, and one candle is replaced with a novel scent. The mice are reintroduced and the number of visits and total time per candle is recorded as before. Testing is also performed with visual stimuli, by placing identical toy brick towers at either corner and then replacing with a different toy brick tower in the testing phase, using the same experimental paradigm monitoring for the number of investigations.

Quantitative RT-PCR. RNA extraction, cDNA synthesis and RT-qPCR analysis are performed on hippocampi and cortical samples from mouse brains as previously described. Primers used to amplify Casp8, GFP, p16^{Ink4a}, p19^{Arf}, p21, Pai1, Il-6, Il-1b and Cd11b are as previously described. The following additional primers are used: Gfap forward 5′-CCTTCTGACACGGATTTGGT-3′, reverse 5′-TAAGCTAGCCCTGGACATCG-3′; S100b forward 5′-CCGGAGTACTGG TGGAAGAC-3′, reverse 5′-GGACACTGAAGCCAGAGAGG-3′; Agp4 forward 5′-TGAGCTCCACATCAGGACAG-3′, reverse 5′-TCCAGCTCGATCTT TTGGAC-3′; Cx3cr1 forward 5′-GTTCCAAAGGCCACAATGTC-3′, reverse 5′-TGAGTGACTGGCACTTCCTG-3′; Olig2 forward 5′-CCCCA GGGATGATCTAAGC-3′, reverse 5′-CAGAGCCAGGTTCTCCTCC-3′; Nef1 forward 5′-AGGCCATCTTGACATTGAGG-3′, reverse 5′-GCAGAATGCAGAC ATTAGCG-3′; Tbp forward 5′-GGCCTCTCAGAAGCATCACTA-3′, reverse 5′-GCCAAGCCCTGAGCATAA-3′. Expression for all experiments is normalized first to Tbp.

Immunohistochemistry and immunofluorescence staining. Mice are transcar-dially perfused as described in ‘Senescence-associated β-galactosidase transmission electronmicroscopy (Gal-TEM)’.Brains are stored in 4% PFA overnight at 4° C. and then cryoprotected by incubating in a 30% sucrose solution for 48 h at 4° C. Samples are sectioned into 30-μM-thick coronal sections and stored in antifreeze solution (300 g sucrose, 300 ml ethylene glycol, 500 ml PBS) at −20° C. Nissl staining (bregma-2.1 to -2.4 mm), thioflavin S staining (bregma-1.4 to -1.6 mm), and phospho-tau S202/S205 (Thermo Fisher, MN1020; 1:500), phospho-tau T231 (Thermo Fisher, MN1040; 1:500), phospho-tau S396 (Abcam, 109390; 1:500), and GFAP (Dako, Z0334; 1:500) and IBA1 (Novus, NB100-1028; 1:100) immunohistochemistry staining (bregma 1.6 to 1.0 mm and lateral 2.0 to 2.7 mm) is performed on free-floating sections as described. NeuN staining (EMD, MAB377; 1:200) of five sections (between bregma-1.3 to -2.5) to measure the dentate gyrus area is performed as previously described. For cellular proliferation assays, mice are injected with EdU (Carbosynth, NE08701; 75 mg kg⁻¹) intraperitoneally 24 h before euthanasia. Imaging of EdU-positive cells (lateral 0.75 to 1.25 mm) is performed following the manufacturer's instructions (Invitrogen Click-iT EdU Alexa Fluor 488 Imaging Kit, C10337). IBA1 (Wako, 019-19741; 1:500) immuno-fluorescent staining and IBA1/EdU colocalization assessments are performed as previously described²⁸. TUNEL staining (lateral 0.75 to 1.25 mm) is performed according to the manufacturer's instructions (Roche In situ Cell Death Detection Kit, Fluorescein: 11684795910). Thioflavin S, EdU/IBA1 colocalization, and in vivo TUNEL-stained images are acquired on a Zeiss LSM 780 confocal system using multi-track configuration.

Single-cell preparation and FACS. Dissociation of cerebral tissue is performed using the Adult Brain Dissociation kit from Miltenyi (MACS, 130-107-677), according to the manufacturer's instructions. Samples are then incubated with a viability dye, LIVE/DEAD Aqua (Invitrogen, L34966; 1:250) followed by incubation with CD11b eFluor 450 (eBioscience, 48-0112-80, 1:100), CD45 APC eFluor 780 (eBioscience, 47-0451-82; 1:200), Glast1 PE (Miltenyi Biotec, 130-095-821; 1:100), O1 AF 700 (R&D Systems, FAB1327N-100UG; 1:100) and CD56 APC (R&D Systems, FAB7820A; 1:100). These samples are then sorted using a FACSAria Ilu SORP (BD Biosciences), with gating parameters created using FACSDiva 8.0.1 (BD Biosciences). A precise gating strategy is used to maximize the purification of each isolated cell population. In brief, populations are isolated first by a negative report of the viability dye indicating the cell is viable (Extended Data 4), followed by a positive report of the desired marker, then negative reports of the other labels used. This strategy allowed for live cells containing only the desired marker to be sorted, while eliminating dead cells. Cells are sorted directly into lysis buffer and RNA is extracted with RNeasy Micro kits according to the manufacturer's instructions (Qiagen, cat no. 74004). cDNA synthesis and RT-qPCR analysis are performed as described above.

IncuCyte tracking of basal and activated astrocytes. To track basal and activated astrocytic response to AP, astrocytes are plated in a 48-well culture plate (10,000 cells per well) and placed into the IncuCyte S3 Live-Cell Analysis System. The IncuCyte System is a time-lapse imaging system that records changes in cell culture through photographic capture of the culture well within the incubator. Cultures are acclimatized to the system for a period of 6 h, then exposed to medium containing IFNγ (R&D Systems, 285-IF; 200 ng ml⁻¹), LPS (Sigma, L2654; 100 ng ml⁻¹) or a combination of both for a period of 24 h to induce an inflammatory response³⁵. Cells are also plated on 10-well slides and processed in tandem for immunofluorescence staining to verify activation status with anti-GFAP (DAKO, Z0334; 1:500) and counterstained with DAPI (Invitrogen, D1306; 1:1,000). After activation, astrocytes are exposed to COMPOUND SELECTED FROM THE TABLE 1 ab and Table 1c (Clontech, 635059; 10 nM or 100 nM) for a period of 24 h. The IncuCyte-captured phase images of each culture well are taken every 30 min over this period using the following settings: segmentation adjustment: 0.8, hole fill: 450, adjust size (pixels): −1, Minimum area (μm²): 0.1. The phase confluency difference is calculated by subtracting the final phase confluency of each image from its initial value.

More details on the protocol can be found in Bussian, T. J., Aziz, A., Meyer, C. F. et al. Clearance of senescent glial cells prevents tau-dependent pathology and cognitive decline. Nature 562, 578-582 (2018). https://doi.org/10.1038/s41586-018-0543-y

### Example 30

### Arthritis

Definitions for this Example: ABTS 2,2′-azino-bis 3-ethylbenzothiazoline-6-sulphonic acid AF-488 Alexa Fluor 488 AP1 activator protein-1 APC antigen-presenting cell APC allophycocyanin bCII bovine collagen type II CD cluster of differentiation CFA complete Freund's adjuvant CIA collagen-induced arthritis DC dendritic cell dLN draining lymph node DMARD disease modifying anti-arthritic drug EDTA ethylenediamine tetraacetate ELISA enzyme-linked immunosorbent assay FACS fluorescence-activated cell sorting FITC fluorescein isothiocyanate H&E hematoxylin and eosin IFNγ interferon-gamma Ig immunoglobulin IKK inhibitory kappa-B (I-kB) kinase IL interleukin IACUC Institutional Animal Care and Use Committee MAPK mitogen-activated protein kinase MTX methotrexare NF-κB nuclear factor kappa-B NSAID nonsteroidal anti-inflammatory drug PBMC peripheral blood mononuclear cells PBS phosphate-buffered saline PE phycoerythrin PGE2 prostaglandins RA rheumatoid arthritis TAK1 transforming growth factor-β activator kinase-1 TCR T-cell receptor TGF-β transforming growth factor-β TNFα tumor necrosis factor-alpha

In one example, CIA-induced DBA/1J mice are treated with compound selected from the table 1 ab and Table 1c or methotrexate (MTX) for 5-weeks.

In some other examples, CIA-induced DBA/1J mice are treated with compound selected from the table 1 ab and Table 1c or methotrexate (MTX) by protocol selected from the Example 1.

Arthritis severity is evaluated by arthritic score and joint histopathology. Draining lymph node (dLN), joint and peripheral-blood mononuclear-cell (PBMC) counts, and activation/localization of T-/B-lymphocytes, dendritic cells (DCs) and neutrophils are examined by FACS analysis. Serum anti-type-II-collagen-(CII) antibody levels and cultured-splenocyte and serum cytokines are also evaluated. Compound selected from the table 1 ab and Table 1c reduces CIA-induced arthritic score, histopathology and leukocyte counts. Besides, compound selected from the table 1 ab and Table 1c suppresses CIA-activated T-cells including CD3+, CD3+/CD69+, CD8+, CD4+ and CD4+/CD25+ in dLNs or joints. It also modifies CD19+ or CD20+/CD23+ (B-cells), MHCII+/CD11c+ (DCs) and Gr-1+/CD11b+ (neutrophil) subpopulations. It further depresses total anti-CII-IgG, anti-CII-IgG1 and anti-CII-IgG2a antibody productions. Moreover, while IFN-γ and IL-10 are not affected, compound selected from the table 1 ab and Table 1c suppresses CIA-induced serum TNF-α, IL-1β and IL-6 levels. Compound selected from the table 1 ab and Table 1c also blocks IFN-γ, IL-17 and IL-6 cytokines while it does not affect IL-10 but enhances IL-4 in splenocytes. These results show that compound selected from the table 1 ab and Table 1c attenuates arthritis severity, modifies leukocyte activations in dLNs or joints, and restores serum and splenocyte cytokine imbalances. Compound selected from the table 1 ab and Table 1c have immunomodulatory and anti-inflammatory properties with the capacity to ameliorate the inflammatory response in CIA-mice.

In some examples, compound selected from the table 1 ab and Table 1c reduces severity of arthritis in CIA mice. DBA1J mice with established CIA are injected either with phosphate-buffered saline (control) or 0.2 mg/kg methotrexate intraperitoneally or with oral dose of compound selected from the table 1 ab and Table 1c 30 or 90 mg/kg daily for 5 weeks after day 29 post-immunization.

In some examples, compound selected from the table 1 ab and Table 1c reduces severity of arthritis in CIA mice. DBA1J mice with established CIA are injected either with phosphate-buffered saline (control) or 0.2 mg/kg methotrexate intraperitoneally or are administered the compound selected from the table 1 ab and Table 1c daily for 5 weeks after day 29 post-immunization by the root of administration and dosage selected from the Example 1. In some examples, compound selected from the table 1 ab and Table 1c reduces severity of arthritis in CIA mice. DBA1J mice with established CIA are injected either with phosphate-buffered saline (control) or 0.2 mg/kg methotrexate intraperitoneally or are administered the compound selected from the table 1 ab and Table 1c after day 29 post-immunization by the protocol selected from the Example 1.

Arthritic score is assessed for the indicated weeks after the onset of arthritis. Paw joints are obtained at the end of the experiment as indicated in the Materials and methods section. Joint tissues should be formalin-fixed, decalcified, sectioned, and stained with hematoxylin-eosin stains and analyzed as naïve unimmunized, vehicle CIA-induced, 30 mg/kg compound selected from the table 1 ab and Table 1c (or, in other example—other dosage suggested in such other example) or 0.2 mg/kg MTX, treated arthritic animals. Extensive leukocyte infiltration and synovial expansion into the articular surface are observed in vehicle-treated animals. Paw joints of CIA-mice demonstrates extensive inflammatory cell infiltration, synovial hyperplasia, cartilage and bone erosions. “Compound selected from the table 1 ab and Table 1c” recipients develops a less cellular infiltration with smooth articular surface and wider articular space, lesser cartilage erosion or bone damage, which is comparable to that of naïve or MTX groups. PBS recipient (untreated) mice revealed no apparent inflammatory cell involvement and normal cartilage integrity. Histology score index calculated from microphoto-graphs is depicted. Histopathological images are magnified at 50×. n=20 for each group.

CIA induction is performed in accordance with nature protocol (Brand D D, Latham K A, Rosloniec E F. Collagen-induced arthritis. Nat Protoc 2007; 2:1269-75.) Briefly, bovine collagen type II (bCII) (Chondrex, Inc., WA, USA), is dissolved in 10 mM acetic acid to 2 mg/ml. This solution is then emulsified in equal volumes of complete Freund's adjuvant (CFA) containing 250 μg/mouse heat-killed Mycobacterium tuberculosis H37Ra (Difco laboratories Inc., MI, USA). Then, 200 μl of the emulsion is injected intradermally at the base of the tail of each mouse on day 0. At day 21 after primary immunization, mice are given booster doses of 200 μl of CII in incomplete Freund's adjuvant (IFA).

### Animals

Male DBA/1J mice (8-weeks old and 20-22 g weight) are obtained from (The Jackson Laboratory; Bar Harbor, Maine, USA), and housed under specific pathogen free conditions with free access to feed and water. Mice are acclimated for two weeks before use and all in vivo experiments are carried out in a specific pathogen-free facility. The experiments are conducted according to internationally accepted guidelines on the use of laboratory animals.

### Collagen-Induced Arthritis

CIA induction is performed in accordance with nature protocol known in the art. Briefly, bovine collagen type II (bCII) (Chondrex, Inc., WA, USA), is dissolved in 10 mM acetic acid to 2 mg/ml. This solution is then emulsified in equal volumes of complete Freund's adjuvant (CFA) containing 250 μg/mouse heat-killed Mycobacterium tuberculosis H37Ra (Difco laboratories Inc., MI, USA). Then, 200 μl of the emulsion is injected intradermally at the base of the tail of each mouse on day 0. At day 21 after primary immunization, mice are given booster doses of 200 μl of CII in incomplete Freund's adjuvant (IFA).

### Treatment

In some examples, Fifty mice are randomly allotted into five groups of 10 each. In one example, Group 1 is used as the non-immunized (normal), whereas mice in Groups 2-5 are immunized on day 0. Group 2 is immunized and untreated vehicle [phosphate-buffered saline (PBS) once daily, orally]; Group 3 is treated with dose of compound selected from the table 1 ab and Table 1c (90 mg/kg/day); Group 4 is treated with low dose of compound selected from the table 1 ab and Table 1c (30 mg/kg/day) both doses are given by oral gavage; and Group 5 is treated with methotrexate (MTX 0.2 mg/kg/day, sc.). In this example, Compound selected from the table 1 ab and Table 1c treatment is initiated at 8 days after booster dose and given orally 5 days/week for 5 weeks.

In other examples, the compound selected from the table 1 ab and Table 1c is administered by protocol suggested in Example 1.

### Clinical Assessment of Arthritis

The clinical arthritis is assessed daily beginning from 18 days of primary immunization and arthritic score is conducted by three in-dependent, blinded examiners for 3 times per week. The severity of arthritis in each of the four paws is graded in a scale of 0-4 according to established protocol and ranked as follows: 0=no evidence of erythema and swelling; 1=erythema and mild swelling confined to the tarsals or ankle joint; 2=erythema and mild swelling extending from the ankle to the tarsals; 3=erythema and moderate swelling extending from ankle to metatarsal joints; and 4=erythema and severe swelling encompass the ankle, foot and digits, or ankylosis of the limb. Each limb is graded, giving a maximum possible score of 16 per animal.

### Hematology of Draining Lymph Node, Joint and Peripheral Blood Mononuclear Total Cell Counts

Body weight gains, total and differential white blood cell counts and red blood cell parameters are determined according to the standard procedure. Total cell counts from draining lymph nodes, joints and peripheral blood mononuclear cells (PBMCs) are conducted before flow cytometric analysis is conducted as indicated below.

Flow cytometry Inguinal and axillary draining lymph node (dLN) cells of 5 arthritic mice from each treatment group are digested with collagenase type D (Roche) (1 mg/ml) and DNase I (Roche) (0.1 mg/ml) in Hanks' balanced salt solution at 37° C. for 30 min and passed through mesh screens to prepare a single-cell suspension. The cells are ished, counted, and incubated with fluorochrome-conjugated antibodies for fluorescence-activated cell sorter (FACS) analysis. To obtain cells from arthritic joints, the limb joints of 5 mice from each treatment group are removed, and synovial tissue is isolated as described. The muscle, patella and ligament are removed before isolating the synovial tissue. The tissue is cut into small pieces and incubated with 1 μg/ml type VI collagenase (Sigma) in Hanks' balanced salt so-lution (Sigma) with 2% FBS and 1 mM ethylenediamine tetraacetate (EDTA). The cells are harvested, ished, and incubated with antibodies for FACS analysis (FACSCalibur; BD Biosciences, San Jose, CA). Characterization of specific subpopulations, developmental stages, and activation status of cells from lymph node or joint is performed by multiparameter FACS analysis. The T- or B-lymphocyte subpopulations, neutrophils and antigen presenting cells are sorted using cell surface marker antibodies. Antibodies from eBioscience, Inc. (San Diego, CA, USA) can be used in this study included the following: anti-CD3-fluorescein isothiocyanate (FITC), anti-CD4-Alexa Fluor 488; anti-B20-FITC, anti-CD8-phycoerythrin (PE), anti-CD4-PE, anti-CD25-allophycocyanin (APC); anti-CD19-PE; anti-Gr-1-PE, anti-CD11b-FITC, anti-MHC-II-PE, anti-CD11c-APC, and anti-CD69-APC.

### Analysis of Anti-Collagen Type II Antibody Production

Mice are bled at the termination of experiment, and sera are analyzed for anti-CII total IgG, IgG1 and IgG2a antibody levels by quan-titative ELISA. Briefly, enzyme-linked immunosorbent assay (ELISA) plates (Thermo Fisher Scientific, NY, USA) are coated with 10 μg/ml of type II collagen dissolved in Tris buffer (50 M Tris, containing 200 mM NaCl, pH 7.4, 0.1% Tween 20), ished and blocked with 3% bovine serum albumin in Tris buffer, and then incubated with serial dilutions of test sera overnight at 4° C. After three ishes, bound total IgG, IgG1 or IgG2a is detected by incubation for 1 h with horseradish peroxidase-conjugated sheep anti-mouse IgG (BD Bio-sciences). After ishing, plates are developed using 2,2′-azino-bis 3-ethylbenzothiazoline-6- sulphonic acid (ABTS) (Roche Diagnostic Systems, IN, USA) as substrate, the reaction is stopp, and the absorbance is then measured at 450 nm in a Spectra Max Plus reader (R & D systems, Minneapolis, MN, USA). A standard serum from arthritic and nonimmunized syngeneic mice is added to each plate in serial dilutions as positive and negative controls, respectively.

Preparation of Spleen Cell Suspensions from CIA Mice

Spleens are aseptically removed from 5 mice in each group and ished with cold PBS and the tissues are minced. Single-cell suspensions are prepared by passing each spleen through a 70-μm-cell strainer with the end of a 10-ml plastic syringe plunger (BD Falcon™; Bedford, MA, USA). The suspensions are then centrifuged on Ficoll-Paque™ at 1200 g for 5 min (Fisher Scientific, PA, USA) and the cell layers are collected. After repeated ishes in PBS, the cells are resuspended in triplicate in 24-well plates cultured with RPMI 1640 medium containing 50 mM 2-mercaptoethanol, 100 U/ml penicillin, 100 μg/ml streptomycin, 50 μg/ml gentamicin and 10% fetal bovine serum (FBS). The cells are dispersed in trypsin, counted and adjusted to 2×cells/ml.

### Analysis of Plasma and Cultured Primary Spleen Cell Cytokine Levels

For the analysis of serum cytokine levels, immunoassays are performed using antibodies against IL-1β, TNF-α, IL-6, IFN-γ, IL-17 and IL-10 of a mouse cytokine immunoassay kit (R&D Systems, MN, USA). Blood samples are collected from all mice via cardiac punctures prior to sacrifice and allowed to clot for 2 h at room temperature, and centrifuged at 2000 g for 20 min at 4° C. to obtain serum. All measurements are made according to the instructions given by the manufacturers of the ELISA kits. For the analysis of splenocyte cytokine levels, single-cell suspensions at a density of 2×1 cells/well are cultured in triplicate to 24-well plates. After 24 h incubation, splenocytes in the absence or presence of compound selected from the table 1 ab and Table 1c (30 mg/kg) or MTX (0.2 mg/kg) are stimulated with 50 μg/ml anti-collagen type II peptide (Cyanogen bromide cleaved peptide 11 (CB11) of bovine type II collagen (Chondrex, Inc., WA, USA)), for 48 h at 37° C. in a 5% CO2-humidified atmosphere. Cytokine (IL-17, IL-6, INF-γ, IL-10 and IL-4) levels in the culture media supernatants are assayed as indicated above in accordance with the manufacturer's recommendations.

### Histological Analysis

For histologic analysis, hind paws are randomly collected from 5 mice of each group, fixed in 10% buffered formalin and decalcified in 15% EDTA. The paws are processed for paraffin embedding with 5-μm sagittal serial tissue sections of whole hind paws and stained with hematoxylin and eosin (H&E) according to standard methods. Histopathological changes are examined by microscopic evaluation and scored in a blinded manner by two independent observers based on cell infiltration, cartilage destruction and bone erosion parameters as described in the art. Histopathologic changes are assessed and scored using a 0 to 3 severity grade scoring system as follows: 0=normal joint structure; 1=mild changes, synovitis, and pannus front with few discrete cartilage focal erosions; 2=moderate changes, accompanying loss of large areas of cartilage, eroding pannus front, and synovial hyperplasia with infiltrating inflammatory cells; and 3=severe synovitis, cartilage and bone erosion and destruction of joint architecture.

### Statistical Analysis

Significant changes in clinical arthritis as a result of drug treatment are determined using a dynamic modeling approach, assuming a linear fit for the slope of arthritis progression for each individual animal (SAS Institute, Inc., Cary, NC, USA). Significant differences in serum cytokines and antibody levels are assessed using the Student's t-test, and Pb 0.05 is considered statistically significant. The clinical and histological score is analyzed using nonparametric analysis; Mann-Whitney test is used when two groups are compared. For differences among study groups, we used Kruskal-Wallis method followed by Dunn's test.

### Result

Effect of Compound Selected from the Table 1 Ab and Table 1c Treatment on Severity of Collagen II-Induced Arthritis

In one example, the systemic arthritis is induced by the injection of bCII and CFA at day 0, plus a booster immunization on day 21 in DBA/1J mice. In some examples, the treatment by compound selected from the table 1 ab and Table 1c is initiated after 8 days of booster dose and assessed the severity of the arthritis with an established arthritic score system. In some examples, one will not detect any overt toxicity or lethality caused by daily compound selected from the table 1 ab and Table 1c treatment evidenced from no difference in body weight gain, complete blood count and red blood cell parameters between the treatment groups. The arthritic scores, indicate that arthritis is progressed rapidly in vehicle-treated mice and the maximal clinical symptoms are achieved at 3 weeks after the onset of arthritis. Compound selected from the table 1 ab and Table 1c shows a decrease in the severity of paw swelling and reduction in arthritis score and this pharmacological action appears to be dose-dependent.

In some examples, The arthritic scores of the mice treated with high (90)- and low (30 mg/kg) doses of compound selected from the table 1 ab and Table 1c are 60-70 and 52-59% decrease in the maximal arthritic scores. MTX (0.2 mg/kg), which is used as control to our test com-pound compound selected from the table 1 ab and Table 1c, exhibited strong inhibitory effect on the development of arthritis.

### 3.2. Histology of the Severity of Joint Inflammation and Cartilage Destruction

CIA and RA are well characterized with synovial hyperplasia, synovitis, pannus formation, and cartilage and bone erosion in the joint. To confirm the protective effects of compound selected from the table 1 ab and Table 1c, histological analysis is carried out on the mouse hind paws. Consistent with the trend toward reduced paw swelling, there is notably a decreased histological signs of inflammation, and cartilage and bone destruction. The vehicle treatment group had serious cellular infiltration, synovial hyperplasia, cartilage damage and bone erosion with a high cumulative histological score, whereas significantly diminished signs of the above indicated pathological lesions, are observed in compound selected from the table 1 ab and Table 1c- and MTX-treated groups compared to vehicle group. This suggests that, in addition to the reduction in inflammatory infiltration, compound selected from the table 1 ab and Table 1c administration could attenuate the inflammatory response of infiltrating/proliferating synovial cells.

### 3.3. Characterization of Specific Subpopulations, Developmental Stage and Activation Status of Isolated Lymph Node or Joint Cells

To better understand how compound selected from the table 1 ab and Table 1c might influence the relative subpopulation of inflammatory cells during the onset of CIA, one can examine the distribution of B and T-lymphocytes in mice after the disease progression. As compared to naive control, the total cell count of CIA mice is increased and compound selected from the table 1 ab and Table 1c treatment exerts a reduction in cell count from the dLNs, joint and PMN cells and this effect is not different from that of MTX group. One can also address the possibility that compound selected from the table 1 ab and Table 1c may regulate the distribution, activation or differentiation of distinct T- and B-cell subsets. The relative population of CD4 and CD8 T-lymphocyte proliferation is increased in dLN of CIA mice with the higher proportion of CD4 T-lymphocytes. While the decrease in CD8 T-cells is significant in compound selected from the table 1 ab and Table 1c or MTX treated groups, the difference in CD4 T-cell subpopulations is not statistically significant. Similarly, CIA-induced CD4/CD25 double positive (black bars) and CD3/CD69 double positive (early T-cell activation marker, gray bar) subpopulations are markedly reduced by compound selected from the table 1 ab and Table 1c or MTX treatment. The relative population of T- and B-lymphocytes in the lymphatic tissue of CIA mice is compared and a there are a profound reduction in CIA-stimulated T- and B-cell populations by compound selected from the table 1 ab and Table 1c.

Effect of compound selected from the table 1 ab and Table 1c on the total cell count of dLN, joints and peripheral circulation in CIA mice. Cells are counted manually using hemocytometer from dLN, synovial fluids, or peripheral PBMN as indicated in the Materials and methods section. Compound selected from the table 1 ab and Table 1c treatment reduces total cell count in the above indicated sites.

In some examples, addition, CIA-induced B-lymphocyte activation (increase in CD20/CD23 double positive cell sub-population) is reversed to normal level by compound selected from the table 1 ab and Table 1c or MTX treatment. co-localization of CIA-activated CD3 expressing T-lymphocytes and Gr-1/CD11b expressing neutrophils in joints are highly diminished in compound selected from the table 1 ab and Table 1c or MTX groups, suggesting the inhibitory effect of compound selected from the table 1 ab and Table 1c in leukocyte infiltration to the synovium, which indicates its beneficial therapeutic property. Similarly, CIA-induced upregulation of MHCII/CD11c expressing antigen presenting cells (APCs) in the dLN is highly reduced in compound selected from the table 1 ab and Table 1c or MTX treated CIA-mice.

Compound Selected from the Table 1 ab and Table 1c Treatment Inhibits Humoral Collagen-Specific Immunity

In CIA mice, the activated dendritic cells (DCs) migrate to dLNs and present the processed CII peptide on appropriate MHC class II molecules to naïve T cells. Engagement of the activated DCs and T-cells creates an environment in which adaptive immunity to CII is induced. The compound selected from the table 1 ab and Table 1c treatment resulted in a significant suppression of anti-CII autoantibody production. The levels of anti-bCII total IgG, IgG1 and IgG2a are reduced in compound selected from the table 1 ab and Table 1c or MTX-treated groups. The result suggests that compound selected from the table 1 ab and Table 1c treatment of mice with chronic arthritis resulted in a reduction in the serum levels of anti-CII IgG antibodies, which correlates with its effect in activated B-cells.

Compound Selected from the Table 1 ab and Table 1c Modulates Serum Cytokine Levels in CIA Mice

Levels of pro-inflammatory and anti-inflammatory cytokines in serum are analyzed using a multiplex immunoassay on day 48 after immunization with CIA. Consistent with the joint swelling, TNF-α, IL-1B, IL-6, and IL-17 in the vehicle-treated CIA mice are systemically over-produced in serum. The elevated cytokine levels in mice treated with compound selected from the table 1 ab and Table 1c are decreased progressively in a manner correlated positively with the degree of joint swelling in individual animals. In the model, the raised serum IL-1β levels in CIA mice are markedly increased. In mice treated with compound selected from the table 1 ab and Table 1c or MTX, serum IL-1β levels are significantly decreased compared with the vehicle treated CIA group). Also, compound selected from the table 1 ab and Table 1c reduces serum levels of TNF-α and IL-6 However, in some examples, inhibitory effect of compound selected from the table 1 ab and Table 1c to serum levels of IFN-γ is not significant while it slightly increased IL-10 levels in the serum of CIA mice, suggesting the shift of Th1/Th2 balance towards the anti-inflammatory cytokine signature Th2 cells that would be expected to be clinically beneficial.

Compound Selected from the Table 1 ab and Table 1c Influences the Balance of Cytokines in CIA-Derived Cultured and Anti-CII Peptide-Treated Splenocytes

Since an imbalance between pro- and anti-inflammatory cytokine activities favors the induction of autoimmunity, chronic inflammation and thereby joint damage, one, in addition to serum levels also, can examine whether such an imbalance could be modulated by compound selected from the table 1 ab and Table 1c treatment in cultured and C-II peptide stimulated-splenocytes isolated from CIA mice. In some examples, the compound selected from the table 1 ab and Table 1c decreases the production of proinflammatory cytokines including Il-17, IL-6 and IFN-γ. In some examples, compound selected from the table 1 ab and Table 1c promotes the anti-inflammatory cytokine, IL-4 production while it does not affect the level of another anti-inflammatory cytokine, IL-10. This suggests that compound selected from the table 1 ab and Table 1c may modulate the balance of cytokines within a complex regulatory network related to specific immunological processes that promote autoimmunity, chronic inflammation and tissue destruction. More Details on the protocol can be found in Endale M, Lee W M, Kwak Y S, Kim N M, Kim B K, Kim S H, Cho J, Kim S, Park S C, Yun B S, Ko D, Rhee M. Torilin ameliorates type II collagen-induced arthritis in mouse model of rheumatoid arthritis. Int Immunopharmacol. 2013 June; 16 (2): 232-42. doi: 10.1016/j.intimp.2013.04.012. Epub 2013 Apr. 24. PMID: 23623942.

### Example 31

Reducing Cellular Senescence in Human Dermal Fibroblasts (HDF) from Hutchinson-Gilford Progeria (HGPS) Patients.

In some examples, Compound selected from the table 1 ab and Table 1c is administered by mice by protocol selected from the Example 1. In some examples, Compound selected from the table 1 ab and Table 1c is administered by human patients by protocol selected from the Example 10.

Compound selected from the table 1 ab and Table 1c effectively decreases HDF senescence induced by HGPS, chronological aging, ultraviolet-B radiation, and etoposide treatment, without inducing significant cell death, and likely by modulating longevity and senescence pathways. One can further validate the effectiveness of Compound selected from the table 1 ab and Table 1c using human skin equivalents and skin biopsies, where Compound selected from the table 1 ab and Table 1c promotes skin health and reduces senescent cell markers, as well as the biological age of samples, according to the Skin-Specific DNA methylation clock, MolClock. Compound selected from the table 1 ab and Table 1c protects fibroblasts from acute UVB and etoposide-induced cellular senescence Ultraviolet-B (UVB) exposure is recognized as a relevant driver of skin aging and is also an effective strategy to induce premature cellular senescence in vitro. To determine whether Compound selected from the table 1 ab and Table 1c could also protect skin cells from extrinsic inducers of cellular senescence, one can treat UVB-exposed HDF with Compound selected from the table 1 ab and Table 1c and assesses cellular senescence markers.

UVB doses of 0.05 J/cm2 and 0.1 J/cm2 effectively induce dose-dependent cellular senescence, as can be verified by SA-BGal staining relative to cell count, and reduces overall cell count). UVB-exposed fibroblasts from 30 and 79 year old donors treated with increasing concentrations of Compound selected from the table 1 ab and Table 1c present decreases SA-BGal staining/nuclei and prevents UVB-induced cell death. To support the reduction in senescence one can measure gene expression of cells exposed to 0.1 J/cm2 and treated with Compound selected from the table 1 ab and Table 1c. UVB-damaged 30 yr fibroblasts treated with Compound selected from the table 1 ab and Table 1c displays a significant decrease in the mRNA expression of genes related to aging, inflammation and SASP (B2M, IL-6, and IL-8), and a trend towards a decrease in senescence (P16). In UVB-dosed 79 yr HDFs treated with Compound selected from the table 1 ab and Table 1c, a trend is observed for decreased gene expression of P16 and B2M but no change in inflammatory markers is observed with Compound selected from the table 1 ab and Table 1c treatment. On the protein level, Compound selected from the table 1 ab and Table 1c also decreases the levels of the senescence marker P21 in 30 yr and 79 yr HDFs after treatment with an acute dose of UVB 2E. Compound selected from the table 1 ab and Table 1c reduces biological age in skin models. In order to assess whether Compound selected from the table 1 ab and Table 1c treatment would effectively decrease cellular senescence in more complex and representative skin samples, one can replicate skin aging in vitro by building 3D skin equivalents using fibroblast and keratinocyte cultures derived from elderly donors. The aged phenotype can be confirmed by measuring the overall structure and 65 quality of the skin, which decreased according to the donor age. Gene expression analysis reveals that, as cell donor age increased, P16 and IL-8 mRNA expression significantly increased, while Ki67 and HAS-2 mRNA expression tended to decrease. Rapamycin is a long studied molecule affecting mTOR/nutrient signaling and has recently been shown to decrease P16 levels of aging skin 21, therefore it is chosen as a positive control of senotherapeutic effect in aging skin models. When added to culture media, Compound selected from the table 1 ab and Table 1c promotes the maintenance of the overall structure of the 3D skin equivalents created with cells from 48 yr old donors, as analyzed by H&E staining, whereas Rapamycin caused a detrimental effect in overall skin structure, including a thinner and more disorganized epidermis. The effect of both molecules is quantified according to morphological changes analyzed through an unbiased skin score and shows that, while Compound selected from the table 1 ab and Table 1c treatment increases the score, Rapamycin treatment lead to a decrease. The mRNA expression of the Compound selected from the table 1 ab and Table 1c-treated epidermis from 3D skin equivalents built from donors aged 32, 48, and 60 years exhibits a significant decrease in P16 and a trend towards decreases IL6, in addition to a significant increase in Keratin 1 (a marker of keratinocyte terminal differentiation) and 14 (a marker of non-differentiated, proliferative keratinocytes). Rapamycin induces a significant increase in P16 expression, a trend towards increased expression of inflammatory markers (IL6 and IL8), and a significant decrease in Keratin 1 gene expression levels 3B). In the dermis, Compound selected from the table 1 ab and Table 1c treatment promotes a significant reduction in B2M gene expression, a pro-aging factor, as well as in the expression of IL8. Rapamycin treatment induces no significant changes in these markers and increases Matrix Metalloproteinase-1 (MMP1) gene expression, indicative of breakdown of the extracellular matrix 3C). Rapamycin and Compound selected from the table 1 ab and Table 1c reduce gene expression of Hyaluronidase-1 (HYAL1), which plays a role in the degradation of hyaluronic acid.

In some examples, Unlike Rapamycin, Compound selected from the table 1 ab and Table 1c treatment stimulates increased gene expression of Collagen 1 and Hyaluronan Synthase-2 (HAS2) 3C). Therefore, compared to Rapamycin, Compound selected from the table 1 ab and Table 1c treatment of 3D human skin equivalents improvs numerous markers of skin health and longevity.

To support the results observed with Compound selected from the table 1 ab and Table 1c treatment in human skin equivalents, one can treat ex vivo skins from donors aged 35, 55, and 79 years. Histological imaging can show an increase in epidermal area with increasing concentration of Compound selected from the table 1 ab and Table 1c and no significant changes are observed with Rapamycin treatment compared to control. In the epidermis of treated skins, both Compound selected from the table 1 ab and Table 1c and Rapamycin decreases P16 mRNA expression yet only Compound selected from the table 1 ab and Table 1c treatments decreases B2M expression. IL8 and tyrosinase (TYR) gene expression are reduced in all treatments though an increase in Keratin 1 and 14 is only observed with Compound selected from the table 1 ab and Table 1c treatment. In the dermis, Compound selected from the table 1 ab and Table 1c treatment more drastically decreased P16 mRNA expression compared to Rapamycin treatment and only the higher concentration of Compound selected from the table 1 ab and Table 1c is able to reduce B2M gene expression. Rapamycin treatment reduces both IL8 and HYAL1 gene expression and, similarly to Compound selected from the table 1 ab and Table 1c, is able to increase HAS2 expression. Compound selected from the table 1 ab and Table 1c treatment also increases dermal Collagen 1 and the proliferation marker Ki67 in dermis though does increase HYAL1 and MMP1 gene expression in the higher concentration 3G).

In order to have a more in depth understanding of how both Compound selected from the table 1 ab and Table 1c and Rapamycin treatments might alter skin's biological age, the 79 year old ex vivo skin biopsies are processed for DNA isolation and methylome analysis. Using the Skin-Specific DNA methylation algorithm (Boroni, M. et al. Highly accurate skin-specific methylome analysis algorithm as a platform to screen and validate therapeutics for healthy aging. Clinical Epigenetics vol. 12 (2020), one can observe that while Rapamycin treatment did not generate any significant alterations (p=0.21), Compound selected from the table 1 ab and Table 1c treatment reduces the DNA methylation age (p<0.01) 3H).

### Cell Culture

Primary human dermal fibroblasts derived from HGPS donors are obtained from The Progeria Research Foundation Cell and Tissue bank. Healthy normal human dermal fibroblasts and human keratinocytes are either purchased from Coriell Institute for Medical Research (Camden, NJ), MatTek Life Science (Ashland, MA), or isolated from ex vivo human skin explants obtained from ZenBio (Research Triangle, NC). The cells can be purchased from Coriell Institute for Medical Research included HDF 71 yr (AG05811, XX, arm, caucasian), HDF 84 yr (AG11725, XX, arm, caucasian) and HDF 90 yr (AG08712, XX, arm, caucasian).

Cells can be purchased from MatTek Life Science included HDF 60 yr (F13400A, XX, african-american), keratinocytes 60yr (K13400A, XX, african-american), neonatal HDFs (F90800, XY, foreskin, caucasian), neonatal keratinocytes (K90800A, XY,foreskin, caucasian).

All other cells are isolated from ex vivo human skin obtained from ZenBio (Research Triangle, NC). All skin samples are from XX donors, caucasian, and abdomen area. The ages included 30, 35, 41, 48, 55 and 79 years. The cell isolation is performed as described by Zonari et al. Briefly, the tissue samples are cut into small pieces of 0.5 cm2 and incubated in PBS containing dispase (2.5 U/mL, BD Biosciences) overnight at 4° C. The epidermis is then mechanically separated from the dermis and incubated in 0.5% trypsin-EDTA (Gibco, USA) for 7 min at 37° C. to isolate the keratinocytes. The cells are separated from the remaining tissue using a 100 mm pore size cell strainer (BD Biosciences, USA) and the cell suspension is centrifuged at 290 g for 5 min. Human keratinocytes are seeded at a density of 100,000 cells/cm in Keratinocyte Serum Free Medium (KSFM) supplemented with Epidermal growth factor and Bovine pituitary extract (Gibco). For the isolation of HDF, the dermis separated from the epidermis is incubated in PBS containing collagenase IA (250 U/mL, Sigma) for 3 hr at 37° C. The HDF are separated from the remaining tissue using a 100 mm pore size cell strainer, centrifuged at 300 g for 5 min and seeded at a density of 50,000 cells/cm in Dulbecco's Modified Eagle Medium (Invitrogen, Carlsbad, CA), supplemented with 10% v.v. Fetal Bovine Serum (FBS; VWR) and 1% v.v. Penicillin-Streptomycin (Invitrogen).

### Example 32

### Topical Formulation 1

### Pharmaceutical Composition for Topical Application.

Compound selected from the table 1 ab and Table 1c, Water (Aqua), Glycerin, Calycophyllum spruceanum Bark Oleic Extract (Pau Mulato), Sorbitan Olivate, Cetearyl Olivate, Squalane, Carapa guianensis Seed (Andiroba) Oil, Vitis Vinifera (Grape) Seed Oil, Pentaclethra macroloba (Pracaxi) Oil, Rosa Canina (Rose hips) Fruit Oil, Prunus Domestica (Plum) Seed Oil, Caprylhydroxamic Acid, Glyceryl Caprylate, Decapeptide-52*, Hyaluronic Acid, Sodium Hyaluronate, Sodium Hyaluronate Crosspolymer, Niacinamide, Allantoin, Bentonite, 65 Tocopheryl Acetate, Tocopherol, Cellulose, Cetyl Palmitate, Sorbitan Palmitate, Xanthan Gum, Potassium Sorbate, Phenoxyethanol, Sorbic Acid, Caprylyl Glycol, Tetrasodium Glutamate Diacetate.

### Topical Formulation 2

### Pharmaceutical Composition for Topical Application.

Compound selected from the table 1 ab and Table 1c-100 mg, Water (Aqua)-100 mg,

Every other substance 10 mg each: Glycerin, Calycophyllum spruceanum Bark Oleic Extract (Pau Mulato), Sorbitan Olivate, Cetearyl Olivate, Squalane, Carapa guianensis Seed (Andiroba) Oil, Vitis Vinifera (Grape) Seed Oil, Pentaclethra macroloba (Pracaxi) Oil, Rosa Canina (Rose hips) Fruit Oil, Prunus Domestica (Plum) Seed Oil, Caprylhydroxamic Acid, Glyceryl Caprylate, Hyaluronic Acid, Sodium Hyaluronate, Sodium Hyaluronate Crosspolymer, Niacinamide, Allantoin, Bentonite, Tocopheryl Acetate, Tocopherol, Cellulose, Cetyl Palmitate, Sorbitan Palmitate, Xanthan Gum, Potassium Sorbate, Phenoxyethanol, Sorbic Acid, Caprylyl Glycol, Tetrasodium Glutamate Diacetate.

### Example 33

Fur (alopecia), liver and renal function improvement by compound selected from the table 1 ab and Table 1c

In some other examples, mice are treated with compound selected from the table 1 ab and Table 1c by protocol selected from the Example 1.

COMPOUND SELECTED FROM THE TABLE 1 ab and Table 1c counteracts frailty and loss of renal function in naturally aged mice as well improves fur.

One can test if compound selected from the table 1 ab and Table 1c target senescence and tissue homeostasis in normal mice that are allowed to age naturally. The biological variation in p16-driven senescence is substantial in aged p16::3MR, compared to young Xpd^{TTD/TTD}-p16::. The variation in running wheel activity is too large to perform meaningful experiments. Nonetheless, while not influencing platelet levels, COMPOUND SELECTED FROM THE TABLE 1 ab and Table 1c significantly reduces p16-driven RLUC, and improve fur density and responsiveness. Furthermore, in the kidneys of these mice COMPOUND SELECTED FROM THE TABLE 1 ab and Table 1c increases the number of LMNB1 positive cells, reduces IL-6 expression and restores renal filtering capacity measured by decreased plasma Urea. As an extra control, also the plasma levels of a second metabolite indicative of reduced renal function, Creatinine, can be measured. Also this is improved by COMPOUND SELECTED FROM THE TABLE 1 ab and Table 1c, independently confirming the beneficial effect of COMPOUND SELECTED FROM THE TABLE 1 ab and Table 1c on the restoration of renal filtering capacity in naturally aged mice. As can be seen for the Xpd^{TTD/TTD}-p16::3MR mice, administration of COMPOUND SELECTED FROM THE TABLE 1 ab and Table 1c or GCV reduce plasma Urea and Creatinine levels. In some examples, senescent cells are causal for the reduction in renal function in fast aging Xpd^{TTD/TTD} and naturally aged wildtype mice and by selective targeting of high-SASP expressing senescent cells in the tubuli, COMPOUND SELECTED FROM THE TABLE 1 ab and Table 1c can restore kidney homeostasis. In some examples, By inducing TASC, COMPOUND SELECTED FROM THE TABLE 1 ab and Table 1c may thus be a potent drug to restore loss of health after natural aging and is an attractive option to explore further in the battle against those age-related diseases that are at least in part driven by senescence.

Doxorubicin if administered by mice reduces total body weight and induces expression of FOXO4 foci and IL-6 in the liver. These effects are neutralized after sequential treatment with COMPOUND SELECTED FROM THE TABLE 1 ab and Table 1c.

Doxorubicin strongly induce plasma levels of Aspartate Aminotransferase, AST, an established indicator of liver damage. COMPOUND SELECTED FROM THE TABLE 1 ab and Table 1c potently counteracted the Doxorubicin-induced increase in plasma AST.

### Animals

All the mice to be used in this study are of a C57BL/6J background; either wildtype, Xpd^{TTD/TTD} mutated, expressing p16::3MR, or a combination thereof. The individual strains are backcrossed at least 10 times prior to this study. For the combination Xpd^{TTD/TTD}×p16::3MR the F1 generation is used. The mice are used for Doxorubicin-experiments at 10-40 wks of age, for Xpd^{TTD/TTD} vs. wt experiments at 26-60 wks of age and for naturally aged mice at 115-130wks of age. All mice are kept in group housing until the start of the experiment after which they are placed in individual cages containing free access to a running wheel. Both sexes are used throughout the study. Where feasible, littermates of the same sex are used. These are randomly assigned to experimental groups.

### Fur Density Analysis

Xpd^{TTD/TTD} mice show reduced fur density. To score any changes that might occur over time, the phenotype is ranged from 0-4 where 0 is very patched and 4 is wildtype. Each mouse is scored before and after the experiment. The final score is determined as the ratio (final-baseline)/baseline and the % change is subsequently plotted. Following several initial pilot observations, the experiment contains mice from two independent cohorts. For naturally aged mice only males can be included, since one often do not observe significant hair loss in females. In these cohorts, around 80% shows (varying degrees of) loss of hair at the beginning (115+wks), or developed it over the course of the experiment.

### Plasma Values as Measure for Tissue Function

On the indicated time points, whole blood samples are collected in a Microvette with Lithium Heparin (Sarstedt) for plasma separation and spun for 10 min at 4.6×g. The (clear) supernatant is transferred into regular 1.5 ml tubes and spun again for 5 min at 4.6×g. The supernatants are transferred again into 1.5 ml tubes, snap frozen in liquid N₂ and stored at −80° C. [AST] is measured using an AST Activity Assay Kit (Sigma). The samples are incubated with 100 μl reaction mix in a 96 well plate and place at 37° C. The absorbance at 450 nm is determined after 2 minutes for baseline analysis and after 40 minutes for a final analysis. [Urea] is measured using a QuantiChrom Urea Assay Kit (Gentaur). The samples are incubated in 200 μl reaction mix for 10 minutes at room temperature before absorbance is measured at 520 nm. [Creatinine] is measured using Creatinine Assay Kit (Sigma). Samples are incubated with 50 μl reaction mix at 37° C. for 60 minutes and the absorbance is measured at 570 nm. Ratios comparing plasma values after treatment compared to baseline are determined and plotted as % over baseline in scatter plots.

### Kidney Slice Culture

Freshly isolated kidneys are sectioned in 200 M thick slices using a Vibratome (Leica, Eindhoven, the Netherlands). The sections are cultured in Dulbecco's modified eagle medium with 10% FCS at 37° C., 5% CO2 on a shaker (60 rpm). Following incubation with shRNA-containing lentiviral particles, or COMPOUND SELECTED FROM THE TABLE 1 ab and Table 1c, as indicated, the slices are fixed for 30 min in formalin and stored at −80. Subsequently, they are subsectioned to 10 M slices using a Cryostat, placed on a charged microscopy slide and processed for TUNEL positivity.

### Alopecia

In some examples, topical application of compound selected from the table 1 ab and Table 1c is effective in treatment of alopecia (In humans) and fur improvement (in mice and other animals).

In some examples, application of compound selected from the table 1 ab and Table 1c is effective in treatment of alopecia (In humans if administered PO or IV) and fur improvement (in mice and other animals if administered PO or IV or IP).

In some examples, application of compound selected from the table 1 ab and Table 1c is effective in treatment of alopecia (In humans if administered in dosage selected from Example 10 PO or IV) and fur improvement (in mice and other animals if administered in dosage selected from Example 1 PO or IV or IP)

More details on the protocol can be found in Baar M P, Brandt R M C, Putavet D A, et al. Targeted Apoptosis of Senescent Cells Restores Tissue Homeostasis in Response to Chemotoxicity and Aging. Cell. 2017; 169(1):132-147.e16. doi:10.1016/j.cell.2017.02.031.

### Example 34

Lisuride Patch known in the art for transdermal delivery can be a non-limiting example for composition and delivery device for any one of the compounds selected from the table 1 ab and Table 1c.

### Example 35

Any Example of the treatment suggested by this application, wherein the treatment groups of mice is treated each—by the protocol suggested below until the at least one symptom of the respective disease is not alliviated.
1. Compound selected from the table 1 ab and Table 1c, PO administration, every 24 h 0.001 mg/kg
2. Compound selected from the table 1 ab and Table 1c, PO administration, every 24 h 0.005 mg/kg
3. Compound selected from the table 1 ab and Table 1c, PO administration, every 24 h 0.01 mg/kg
4. Compound selected from the table 1 ab and Table 1c, PO administration, every 24 h 0.05 mg/kg
5. Compound selected from the table 1 ab and Table 1c, PO administration, every 24 h 0.5 mg/kg
6. Compound selected from the table 1 ab and Table 1c, PO administration, every 24 h 1 mg/kg
7. Compound selected from the table 1 ab and Table 1c, administration with food, compound should be mixed with feed at dosage 0.01 mg/kg,
8. Compound selected from the table 1 ab and Table 1c, administration with food, compound should be mixed with feed at dosage 0.1 mg/kg,
9. Compound selected from the table 1 ab and Table 1c, administration with food, compound should be mixed with feed at dosage 1 mg/kg,
10. Compound selected from the table 1 ab and Table 1c, administration with food, compound should be mixed with feed at dosage 5 mg/kg,
11. Compound selected from the table 1 ab and Table 1c, administration with food, compound should be mixed with feed at dosage 10 mg/kg,
12. Compound selected from the table 1 ab and Table 1c, administration with food, compound should be mixed with feed at dosage 20 mg/kg,
13. Compound selected from the table 1 ab and Table 1c, administration with food, compound should be mixed with feed at dosage 50 mg/kg
14. injection of Compound selected from the table 1 ab and Table 1c-0,025 mg/kg-1 time a day, IP
15. injection of Compound selected from the table 1 ab and Table 1c-0.25 mg/kg-1 time a day, IP
16. injection of Compound selected from the table 1 ab and Table 1c-0.01 mg/kg 1 time a day, IP
17. injection of Compound selected from the table 1 ab and Table 1c-0.5 mg/kg-1 time a day, IP
18. injection of Compound selected from the table 1 ab and Table 1c-1 mg/kg-1 time a day, IP
19. injection of Compound selected from the table 1 ab and Table 1c-2.5 mg/kg-1 time a day, IP
20. injection of Compound selected from the table 1 ab and Table 1c-5 mg/kg-1 time a day, IP
21. injection of Compound selected from the table 1 ab and Table 1c-10 mg/kg-1 time a day, IP
22. injection of Compound selected from the table 1 ab and Table 1c-25 mg/kg-1 time a day, IP
23. injection of Compound selected from the table 1 ab and Table 1c-50 mg/kg-1 time a day, IP
24. injection of Compound selected from the table 1 ab and Table 1c-1 time a day, IP, in the dosage 10-fold less than LD50.
25. injection of Compound selected from the table 1 ab and Table 1c-1 time a day, IP, in the dosage 50-fold less than LD50.
26. injection of Compound selected from the table 1a-1 time a day, IP, in the dosage 100-fold less than LD50.

### Table 4. Declines

Non-limiting list of parameters which age related change is regarded as age related decline and which can be changed into younger state or stabilized or its further change into the older state delayed by anti-aging interventions of this application.

| Field | Units |
|---|---|
| Standing height | cm |
| Forced expiratory volume in 1-second | litres |
| (FEV1) | |
| Leg fat-free mass (right) | Kg |
| Leg predicted mass (right) | Kg |
| Basal metabolic rate | KJ |
| Forced vital capacity (FVC) | litres |
| Leg fat-free mass (left) | Kg |
| Leg predicted mass (left) | Kg |
| Systolic blood pressure, automated reading | mmHg |
| Heel bone mineral density (BMD) (left) | g/cm2 |
| Heel quantitative ultrasound index (QUI), | |
| direct entry (left) | |
| Whole body fat-free mass | Kg |
| Whole body water mass | Kg |
| Heel bone mineral density (BMD) T-score, | Std. Devs |
| automated (left) | |
| Speed of sound through heel (left) | m/s |
| Sitting height | cm |
| Heel bone mineral density (BMD) (right) | g/cm2 |
| Heel quantitative ultrasound index (QUI), | |
| direct entry (right) | |
| Speed of sound through heel (right) | m/s |
| Heel bone mineral density (BMD) T-score, | Std. Devs |
| automated (right) | |
| Peak expiratory flow (PEF) | litres/min |
| Leg fat percentage (left) | percent |
| Trunk fat-free mass | Kg |
| Leg fat percentage (right) | percent |
| Trunk predicted mass | Kg |
| Hand grip strength (left) | Kg |
| Heel broadband ultrasound attenuation (left) | dB/MHz |
| Heel broadband ultrasound attenuation | dB/MHz |
| (right) | |
| Hand grip strength (right) | Kg |
| Duration to first press of snap-button in each | milliseconds |
| round | |
| Mean time to correctly identify matches | milliseconds |
| Body fat percentage | percent |
| Trunk fat percentage | percent |
| Body mass index (BMI) | Kg/m2 |
| Leg fat mass (left) | Kg |
| Arm fat-free mass (left) | Kg |
| Arm predicted mass (left) | Kg |
| Arm fat-free mass (right) | Kg |
| Haematocrit percentage | percent |
| Arm predicted mass (right) | Kg |
| Waist circumference | cm |
| Leg fat mass (right) | Kg |
| Haemoglobin concentration | grams/ |
| | decilitre |
| Arm fat percentage (left) | percent |
| Ankle spacing width (left) | mm |
| Whole body fat mass | Kg |
| Body mass index (BMI) | Kg/m2 |
| Pulse wave peak to peak time | milliseconds |
| Arm fat percentage (right) | percent |
| Weight | Kg |
| Mean corpuscular volume | femtolitres |
| Trunk fat mass | Kg |
| Pulse wave Arterial Stiffness index | milliseconds |
| Ankle spacing width (right) | mm |
| Platelet crit | percent |
| Red blood cell (erythrocyte) count | 10\hat{ }12 |
| | cells/Litre |
| Mean sphered cell volume | femtolitres |
| Mean platelet (thrombocyte) volume | femtolitres |
| Weight | Kg |
| Arm fat mass (left) | Kg |
| Lymphocyte percentage | percent |
| Neutrophill percentage | percent |
| Arm fat mass (right) | Kg |
| Impedance of leg (left) | ohms |
| Mean reticulocyte volume | femtolitres |
| Platelet count | 10\hat{ }9 |
| | cells/Litre |
| Mean corpuscular haemoglobin | picograms |
| Impedance of leg (right) | ohms |
| Red blood cell (erythrocyte) distribution | percent |
| width | |
| Pulse rate, automated reading | bpm |
| Impedance of whole body | ohms |
| Diastolic blood pressure, automated reading | mmHg |
| Lymphocyte count | 10\hat{ }9 |
| | cells/Litre |
| Number of measurements made | |
| Neutrophill count | 10\hat{ }9 |
| | cells/Litre |
| Monocyte percentage | percent |
| Hip circumference | cm |
| Monocyte count | 10\hat{ }9 |
| | cells/Litre |
| Platelet distribution width | percent |
| Mean corpuscular haemoglobin | grams/ |
| concentration | decilitre |
| Immature reticulocyte fraction | ratio |
| Impedance of arm (right) | ohms |
| Reticulocyte percentage | percent |
| Number of times snap-button pressed | |
| White blood cell (leukocyte) count | 10\hat{ }9 |
| | cells/Litre |
| Pulse rate | bpm |
| High light scatter reticulocyte count | 10\hat{ }12 |
| | cells/Litre |
| Basophill percentage | percent |
| Impedance of arm (left) | ohms |
| Pulse wave reflection index | |
| Eosinophill count | 10\hat{ }9 |
| | cells/Litre |
| Nucleated red blood cell count | 10\hat{ }9 |
| | cells/Litre |
| Eosinophill percentage | percent |
| Basophill count | 10\hat{ }9 |
| | cells/Litre |
| Reticulocyte count | 10\hat{ }12 |
| | cells/Litre |
| High light scatter reticulocyte percentage | percent |
| Nucleated red blood cell percentage | percent |

TABLE 5
Non-limiting list of biomarkers related to mortality risks.
Name

Red blood cell distribution width
Mean reticulocytes volume
Neutrophil number
Alpha-1-acid glycoprotein
White blood cell count
Hemoglobin
Red blood cell count
Monocyte count
Basophil count
Neutrophils percentage
Albumin, serum/plasma
Lymphocyte percentage
Hematocrit
Leukocyte telomere length
Mean sphered cells volume
Very-low-density lipoprotein
Insulin-like growth factor 1
Mean corpuscular hemoglobin
Mean corpuscular volume
Citrate
trans-lycopene
Monocyte percentage
Platelet count
Reticulocytes count
Lymphocyte count
Platelet distribution width
Plateletcrit
Immature reticulocytes fraction
Reticulocytes, high light scatter, percentage
Reticulocytes, high light scatter, number
Reticulocytes percentage
Soluble CD14
Eosinophils percentage
25-hydroxyvitamin D
Adiponectin
Ascorbic acid
Brain natriuretic peptide
C-reactive protein
Cardiac troponin I
Estimated glomerular filtration rate
Fibroblast growth factor-23
Gamma-glutamyltransferase
Glucose
Growth differentiation factor 15
H-FABP
Homeostatic model
assessment of insulin
resistance
Homocysteine
Lipoprotein-associated
phospholipase
A2
activity
N-terminal atrial natriuretic peptide
SUA
Type I collagen degradation
Vitamin A
High-density lipoprotein cholesterol
Klotho
Leptin
Club (a.k.a. Clara) cell secretory protein
Antinuclear autoantibodies
Soluble ST2
Alanine transaminase
Alkaline phosphatase
Alpha-1-antichymotrypsin
Angiopoietin-2
ApoB/ApoA1 ratio
Asymmetric dimethylarginine
C-reactive protein, high-sensitivity
Cardiac troponin T, high-sensitivity
Cholesterol
Creatinine
Cystatin C
Fibrinogen
Glycated hemoglobin
Growth hormone
Homoarginine
Insulin-like growth factor binding protein 1
Interleukin-6
Low-density lipoprotein cholesterol
Lycopene
Mitochondrial DNA copy number
N-terminal pro-brain natriuretic peptide
Neutrophil gelatinase-associated lipocalinin
Osteocalcin
Osteoprotegerin
Phosphorus
Testosterone
Triglycerides
Tumor necrosis factor alpha
Uric acid
alpha-carotene
beta-trace protein
β2-microglobulin
Anion gap, serum albumin adjusted
CD4:CD8 ratio
CD8 cells
Carboxyl-terminal telopeptide of collagen type I
Plasma viscosity
Insulin-like growth factor binding protein 2
Peroxiredoxin 4
Stromal cell derived factor
Carotenoids
Oxygenated carotenoids
Urea
sj/B-TREC ratio
Insulin-like growth factor binding protein 3
Proinsulin
Factor VIIc
IgA to tissue transglutaminase
Bilirubin
Mean platelet volume
Galectin-3
Interleukin-8
Loss of Y chromosome
Soluble tumour necrosis factor receptor 1
Symmetrical dimethyl arginine
T cells
Thyroxine
Cell-free DNA concentration
beta-cryptoxanthin
Basophil percentage
Interleukin-10
Apolipoprotein A-1
Amino-terminal propeptide of type I collagen
Estradiol-to-sex hormone binding globulin ratio
Neutrophilia
Butyrylcholinesterase activity
Reticulocytes number
Parathyroid hormone
Follicle stimulating hormone
Interleukin 1-beta
17beta-E(2)
Amino-terminal compound of procollagen type
III

The method of biological age/DFI evaluation can be used to evaluate compounds and composition of this application for anti-aging use.

### REFERENCES

Whitehead J C, Hildebrand B A, Sun M, Rockwood M R, Rose R A, Rockwood K, Howlett S E. A clinical frailty index in aging mice: comparisons with frailty index data in humans. J Gerontol A Biol Sci Med Sci. 2014; 69(6):621-32
Kane, A. E., et al., Animal models of frailty: current applications in clinical research. Clin Interv Aging, 2016; 11:1519-1529
Burd C E, Sorrentino J A, Clark K S, Darr D B, Krishnamurthy J, Deal A M, Bardeesy N, Castrillon D H, Beach D H, Sharpless N E. Monitoring tumorigenesis and senescence in vivo with a p16(INK4a)-luciferase model. Cell. 2013; 152(1-2):340-51.
Guillon J, Petit C, Toutain B, Guette C, Lelièvre E, Coqueret O. Chemotherapy-induced senescence, an adaptive mechanism driving resistance and tumor heterogeneity Cell Cycle. 2019; 18(19):2385-2397
Krishnamurthy J, Torrice C, Ramsey M R, Kovalev G I, Al-Regaiey K, Su L, Sharpless N E Ink4a/Arf expression is a biomarker of aging. J Clin Invest. 2004; 114(9):1299-307.
Liu Y, Sanoff H K, Cho H, Burd C E, Torrice C, Ibrahim J G, Thomas N E, Sharpless N E. Expression of p16 (INK4a) in peripheral blood T-cells is a biomarker of human aging. Aging Cell. 2009; 8(4):439-48
Chiang A C A, Huo X, Kavelaars A, Heijnen C J. Chemotherapy accelerates age-related development of tauopathy and results in loss of synaptic integrity and cognitive impairment. Brain Behav Immun. 2019; 79:319-325
Chen J, Lau Y F, Lamirande E W, Paddock C D, Bartlett J H, Zaki S R, Subbarao K. Cellular immune responses to severe acute respiratory syndrome coronavirus (SARS-CoV) infection in senescent BALB/c mice: CD4+ T cells are important in control of SARS-CoV infection. J Virol. 2010; 84:1289-301
Interleukin-6 in COVID-19: A Systematic Review and Meta-Analysis https://www.medrxiv.org/content/10.1101/2020.03.30.20048058v1
Anti-IL-6 Agents Suggested by SITC to Treat Patients with COVID-19 https://www.cancernetwork.com/news/anti-il-6-agents-suggested-site-treat-patients-covid-19
Santesmasses et al., 2020, COVID-19 is an emergent disease of aging; medRxiv 2020.04.15.20060095; doi:https://doi.org/10.1101/2020.04.15.20060095
Avchaciov et al., 2020, Identification of a blood test-based biomarker of aging through deep learning of aging trajectories in large phenotypic datasets of mice, bioRxiv 2020.01.23.917286; doi: https://doi.org/10.1101/2020.01.23.917286
Rockwood et al., 2005, A global clinical measure of fitness and frailty in elderly people CMAJ 173 (5) 489-495; DOI: https://doi.org/10.1503/cmaj.050051.
Searle et al., 2008, A standard procedure for creating a frailty index, BMC Geriatrics volume 8, Article number: 24.
Rockwood et al., 2017, A Frailty Index Based On Deficit Accumulation Quantifies Mortality Risk in Humans and in Mice.” Scientific reports vol. 7 43068.
Justice et al., 2016, Frameworks for Proof-of-Concept Clinical Trials of Interventions That Target Fundamental Aging Processes, J Gerontol A Biol Sci Med Sci; 71(11):1415-1423.
Stubbs et al., 2017, Multi-tissue DNA methylation age predictor in mouse. Genome Biol.; 18 (1): 68
Horvath S., 2013, DNA methylation age of human tissues and cell types. Genome Biol. 2013; 14(10):R115
Harrison et al., 2009, Rapamycin fed late in life extends lifespan in genetically heterogeneous mice. Nature. 2009 Jul. 16; 460(7253):392-5
Parks et al, 2012, A procedure for creating a frailty index based on deficit accumulation in aging mice. J Gerontol A Biol Sci Med Sci 2012; 67:217-227
Antoch, et al. 2017, Physiological frailty index (PFI): quantitative in-life estimate of individual biological age in mice. Aging (Albany NY). 2017; 9(3):615-626. doi:10.18632/aging.101206
Rebo et al., 2016, A single heterochronic blood exchange reveals rapid inhibition of multiple tissues by old blood, Nature Communications volume 7, Article number: 13363 (2016)
Miyamoto and Nosé, 2010, Can an Apheresis Therapy become an Effective Method for Anti-Aging Medicine?, Hiroshi Miyamoto, Yukihiko Nosé Sep. 1, 2010, Anti-Aging Medicine 7 (9): 100-106, 2010 (c) Japanese Society of Anti-Aging Medicine
Kellar A, Egan C, Morris D. Preclinical Murine Models for Lung Cancer: Clinical Trial Applications. Biomed Res Int. 2015; 2015:621324. doi: 10.1155/2015/621324.
Gao L, Chen X, Wang Y, Zhang J. Up-Regulation of FSTL3, Regulated by lncRNA DSCAM-AS1/miR-122-5p Axis, Promotes Proliferation and Migration of Non-Small Cell Lung Cancer Cells. Onco Targets Ther. 2020; 13:2725-2738 https://doi.org/10.2147/OTT.S236359
Mukherjee A et al. FSTL3 deletion reveals roles for TGF-beta family ligands in glucose and fat homeostasis in adults. Proc Natl Acad Sci USA. 2007; 104 (4): 1348-1353)
Schneyer A., et al. OR14-01 FSTL3 Neutralizing Antibodies Restore Function to Diabetic Mouse and Human Islets: A New Approach for Treating Diabetes, Journal of the Endocrine Society, Volume 4, Issue Supplement_1, April-May 2020, OR14-01, https://doi.org/10.1210/jendso/bvaa046.1862.

TABLE 1ab
TABLE 1ab

| inchi_keys | chembl_id | pref_name | full_molformula | synonyms | trade_names | Target |
|---|---|---|---|---|---|---|
| PMATZTZNYRCHOR-CGLBZJNRSA-N | CHEMBL160 | CYCLOSPORINE | C62H111N11O12 | Ciclosporin; Cyclosporine; | Capimune; Capsorin; Cequa; | ABCB1 |
| | | | | Cyclosporin A; 27- | Cyclosporine; Deximune; Gengraf; | |
| | | | | 400; ANTIBIOTIC S- | Ikervis; Neoral; Optimmune; | |
| | | | | 7481F1; NSC-290193; OL- | Restasis; Restasis | |
| | | | | 27-400; SANG-35; SDZ-OXL- | multidose; Sandimmun; | |
| | | | | 400 | Sandimmune; Sangcya; Vanquoral | |
| LGGHDPFKSSRQNS-UHFFFAOYSA-N | CHEMBL348475 | TARIQUIDAR | C38H38N4O6 | Tariquidar; XR-9576 | | ABCB1 |
| OCSMOTCMPXTDND-OUAUKWLOSA-N | CHEMBL279785 | MARIMASTAT | C15H29N3O5 | Marimastat; BB 2516; BB- | | ADAM17 |
| | | | | 2516; GI 5712; GI-5712; KB- | | |
| | | | | R 8898; TA 2516; TA-2516 | | |
| YKPYIPVDTNNYCN-INIZCTEOSA-N | CHEMBL75094 | PRINOMASTAT | C18H21N3O5S2 | Prinomastat; AG-3340; KB- | | ADAM17 |
| | | | | R-9896 | | |
| MAVDNGWEBZTACC-HNNXBMFYSA-N | CHEMBL206815 | APRATASTAT | 22N2C17HO6S2 | Apratastat; TMI-005; TMI- | | ADAM17 |
| | | | | 05 | | |
| BLGXFZZNTVWLAY-SCYLSFHTSA-N | CHEMBL15245 | YOHIMBINE | C21H26N2O3 | Yohimbine | | ADRA1A |
| URKOMYMAXPYINW-UHFFFAOYSA-N | CHEMBL716 | QUETIAPINE | C21H25N3O2S | Quetiapine | Atrolak XL; Biquelle | ADRA1A |
| | | | | | XL; Brancico XL; Ebesque | |
| | | | | | XL; Mintreleq XL; Psyquet | |
| | | | | | XL; Seotiapim | |
| | | | | | XL; Seroquel; Seroquel | |
| | | | | | XL; Sondate XL; Tenprolide | |
| | | | | | XL; Zaluron XL | |
| ODQWQRRAPPTVAG-UHFFFAOYSA-N | CHEMBL1628227 | DOXEPIN | C19H21NO | Doxepin; Zonalon; P-3693A | Sinepin; Sinequan | ADRA1A |
| UEQUQVLFIPOEMF-UHFFFAOYSA-N | CHEMBL6437 | MIANSERIN | C18H20N2 | Mianserin; J16.3891 | | ADRA1A |
| XCGSFFUVFURLIX-VFGNJEKYSA-N | CHEMBL442 | ERGOTAMINE | C33H35N5O5 | Ergotamine; NSC- | | ADRA1A |
| | | | | 95090; Ergotamine | | |
| | | | | Tartrate | | |
| OSJJYEUEJRVVOD-UHFFFAOYSA-N | CHEMBL1909072 | PIPAMAZINE | C21H24ClN3OS | Pipamazine | | ADRA1A |
| MRBDMNSDAVCSSF-UHFFFAOYSA-N | CHEMBL597 | PHENTOLAMINE | C17H19N3O | Phentolamine | | ADRA1A |
| BKRGVLQUQGGVSM-KBXCAEBGSA-N | CHEMBL157138 | LISURIDE | C20H26N4O | Lisuride; Lisuride | Revanil 200 | ADRA1A |
| | | | | Maleate; Lysuride | | |
| DRHKJLXJIQTDTD-OAHLLOKOSA-N | CHEMBL836 | TAMSULOSIN | C20H28N2O5S | Tamsulosin; HGP-0412; HIP- | | ADRA1A |
| | | | | 1402; HIP1402 | | |
| CUHVIMMYOGQXCV-NSHDSACASA-N | CHEMBL778 | DEXMEDETOMIDINE | C13H16N2 | Dexmedetomidine; MPV- | Precedex | ADRA1A |
| | | | | 1440 | | |
| JXZZEXZZKAWDSP-UHFFFAOYSA-N | CHEMBL279516 | INDORAMIN | C22H25N3O | Indoramin; WY-21901 | Baratol; Doralese Tiltab | ADRA1A |
| RAPZEAPATHNIPO-UHFFFAOYSA-N | CHEMBL85 | RISPERIDONE | C23H27FN4O2 | Risperidone; LY- | Perseris | ADRA1A |
| | | | | 03004; LY03004; N05AX08; | kit; Risperdal; Risperdal | |
| | | | | R-64,766; R-64-766; R- | Consta; Risperdal Consta Long | |
| | | | | 64766; RCN-3028; RCN3028 | Acting; Risperdal M; Risperdal | |
| | | | | | M-Tab; Risperdal | |
| | | | | | Quicklet; Risperidone | |
| GZKLJWGUPQBVJQ-UHFFFAOYSA-N | CHEMBL12713 | SERTINDOLE | C24H26ClFN4O | Sertindole; LU 23-174; LU- | SerLect | ADRA1A |
| | | | | 23-174 | | |
| HUCJFAOMUPXHDK-UHFFFAOYSA-N | CHEMBL312448 | XYLOMETAZOLINE | C16H24N2 | Xylometazoline | | ADRA1A |
| CNIIGCLFLJGOGP-UHFFFAOYSA-N | CHEMBL761 | NAPHAZOLINE | C14H14N2 | Naphazoline; Nafazair | | ADRA1A |
| BYJAVTDNIXVSPW-UHFFFAOYSA-N | CHEMBL1266 | TETRAHYDROZOLINE | C13H16N2 | Tetryzoline; Tetrahydrozoline; | | ADRA1A |
| | | | | Tyzine | | |
| HCYAFALTSJYZDH-UHFFFAOYSA-N | CHEMBL72 | DESIPRAMINE | C18H22N2 | Desipramine | | ADRA1A |
| WNMJYKCGWZFFKR-UHFFFAOYSA-N | CHEMBL709 | ALFUZOSIN | C19H27N5O4 | Alfuzosin; Uroxatral | | ADRA1A |
| PNCPYILNMDWPEY-QGZVFWFLSA-N | CHEMBL24778 | SILODOSIN | C25H32F3N3O4 | Silodosin; KAD-3213; KMD- | Rapaflo | ADRA1A |
| | | | | 3213 | | |
| VCKUSRYTPJJLNI-UHFFFAOYSA-N | CHEMBL611 | TERAZOSIN | C19H25N5O4 | Terazosin; Hytrin | | ADRA1A |
| KVWDHTXUZHCGIO-UHFFFAOYSA-N | CHEMBL715 | OLANZAPINE | C17H20N4S | Olanzapine; LY- | Arkolamyl; Olanzapine; Zalasta; | ADRA1A |
| | | | | 170053; LY170053 | Zyprexa; Zyprexa Zydis | |
| HDOZVRUNCMBHFH-UHFFFAOYSA-N | CHEMBL285802 | ZOTEPINE | C18H18ClNOS | Zotepine | Zoleptil 100; Zoleptil | ADRA1A |
| | | | | | 25; Zoleptil 50 | |
| RMEDXOLNCUSCGS-UHFFFAOYSA-N | CHE1108 | DROPERIDOL | C22H22FN3O2 | Droperidol; McN-JR- | Dridol; Droleptan; Droperidol; | ADRA1A |
| | | | | 4749; NSC-169874; R-4749 | Inapsine | |
| KLBQZWRITKRQQV-UHFFFAOYSA-N | CHEMBL479 | THIORIDAZINE | C21H26N2S2 | Thioridazine; Mellaril; TP-21 | Mellaril; Mellaril- | ADRA1A |
| | | | | | S; Melleril; Rideril | |
| ZPEIMTDSQAKGNT-UHFFFAOYSA-N | CHEMBL71 | CHLORPROMAZINE | C17H19ClN2S | Chlorpromazine; Chlorpromazine | Elmarin; Largactil; Megaphen; | ADRA1A |
| | | | | Hibenzate; Chlorpromazine | Thorazine | |
| | | | | Phenolphthalinate; | | |
| | | | | Chlorpromazine | | |
| | | | | Tannate; Sonazine; J2.794D; | | |
| | | | | NSC-167745; NSC- | | |
| | | | | 226514; SKF-2601A | | |
| PLDUPXSUYLZYBN-UHFFFAOYSA-N | CHEMBL726 | FLUPHENAZINE | C22H26F3N3OS | Fluphenazine; Permitil; | | ADRA1A |
| | | | | Prolixin; NSC-62323 | | |
| OGHNVEJMJSYVRP-UHFFFAOYSA-N | CHEMBL723 | CARVEDILOL | 26N2C24HO4 | Carvedilol; BM 14.190; BM- | Carvedilol; Coreg; Coreg | ADRA1A |
| | | | | 14-190; BM-14.190; BM- | CR; Eucardic 12.5; Eucardic | |
| | | | | 14190; C07AG02; DQ- | 25; Eucardic 3.125; Eucardic | |
| | | | | 2466; SKF-105517 | 6.25 | |
| WYWIFABBXFUGLM-UHFFFAOYSA-N | CHEMBL762 | OXYMETAZOLINE | C16H24N2O | Oxymetazoline; Ocuclear | | ADRA1A |
| LNEPOXFFQSENCJ-UHFFFAOYSA-N | CHEMBL54 | HALOPERIDOL | C21H23ClFNO2 | Haloperidol; MCN-JR- | Dolpin; Dozic; Fortunan; Haldol; | ADRA1A |
| | | | | 1625; NSC-170973; NSC- | Haldol | |
| | | | | 615296; R-1625 | Solutab; Haloperidol; Kentace; | |
| | | | | | Keselen; Serenace | |
| QZVCTJOXCFMACW-UHFFF AOYSA-N | CHEMBL753 | PHENOXYBENZAMINE | C18H22ClNO | Phenoxybenzamine | | ADRA1A |
| HRRBJVNMSRJFHQ-UHFFFAOYSA-N | CHEMBL142635 | NAFTOPIDIL | C24H28N2O3 | Naftopidil | | ADRA1A |
| RUZYUOTYCVRMRZ-UHFFFAOYSA-N | CHEMBL707 | DOXAZOSIN | C23H25N5O5 | Doxazosin; Cardura; C02CA04; | | ADRA1A |
| | | | | UK-33274 | | |
| OZVBMTJYIDMWIL-AYFBDAFISA-N | CHEMBL493 | BROMOCRIPTINE | C32H40BrN5O5 | Bromocriptine; CB- | Parlodel | ADRA1A |
| | | | | 154; SANDOZ 15-754 | | |
| QZUDBNBUXVUHMW-UHFFFAOYSA-N | CHEMBL42 | CLOZAPINE | C18H19ClN4 | Clozapine; HF- | Clozapine; Clozaril; Fazaclo; | ADRA1A |
| | | | | 1854; Clozapine resolution | Fazaclo Odt; Versacloz; Zaponex | |
| | | | | mixture | | |
| IENZQIKPVFGBNW-UHFFFAOYSA-N | CHEMBL2 | PRAZOSIN | C19H21N5O4 | Prazosin; CP-12299; CP- | | ADRA1A |
| | | | | 122991 | | |
| LUZRJRNZXALNLM-JGRZULCMSA-N | CHEMBL1732 | DIHYDROERGOTAMINE | C33H37N5O5 | Dihydroergotamine; MAP- | | ADRA1A |
| | | | | 0004; MAP0004 | | |
| GDLIGKIOYRNHDA-UHFFFAOYSA-N | CHEMBL415 | CLOMIPRAMINE | C19H23ClN2 | Clomipramine; Anafranil; | | ADRA1A |
| | | | | NSC-169865 | | |
| KRMDCWKBEZIMAB-UHFFFAOYSA-N | CHEMBL629 | AMITRIPTYLINE | C20H23N | Amitriptyline; Amitid; Amitril; | | ADRA1A |
| | | | | Elavil; Endep; Etrafon- | | |
| | | | | A; Etrafon-Forte; Limbitrol | | |
| BCGWQEUPMDMJNV-UHFFFAOYSA-N | CHEMBL11 | IMIPRAMINE | C19H24N2 | Imipramine; Janimine; | | ADRA1A |
| | | | | Pramine; Presamine; Tofranil; | | |
| | | | | Tofranil-PM; NSC- | | |
| | | | | 169866; ORG-2463 | | |
| PHVGLTMQBUFIQQ-UHFFFAOYSA-N | CHEMBL445 | NORTRIPTYLINE | C19H21N | Nortriptyline; Pamelor; NCI- | Allegron; Aventyl; Motipress; | ADRA1A |
| | | | | 169453 | Motival | |
| GXDALQBWZGODGZ-UHFFFAOYSA-N | CHEMBL296419 | ASTEMIZOLE | C28H31FN4O | Astemizole; GNF-Pf- | Hismanal; Pollon-Eze | ADRA1A |
| | | | | 2461; R-43512 | | |
| ZGUGWUXLJSTTMA-UHFFFAOYSA-N | CHEMBL564 | PROMAZINE | C17H20N2S | Promazine; NSC-31447 | | ADRA1A |
| MVWVFYHBGMAFLY-UHFFFAOYSA-N | CHEMBL708 | ZIPRASIDONE | C21H21ClN4OS | Ziprasidone; Geodon; CP- | | ADRA1A |
| | | | | 88059 | | |
| SONNWYBIRXJNDC-VIFPVBQESA-N | CHEMBL1215 | PHENYLEPHRINE | C9H13NO2 | Phenylephrine; Cyclomydril; | | ADRA1A |
| | | | | Duo-Medihaler; AB-101 | | |
| | | | | (PHENYLEPHRINE); J8.601K | | |
| DCSUBABJRXZOMT-UHFFFAOYSA-N | CHEMBL1729 | CISAPRIDE | C23H29ClFN3O4 | Cisapride; Cisapride | Alimix; Prepulsid; Prepulsid | ADRA1A |
| | | | | Monohydrate; Propulsid; R- | Quicklet; Propulsid; Propulsid | |
| | | | | 51619 | Quicksolv | |
| RFWZESUMWJKKRN-UHFFFAOYSA-N | CHEMBL1201216 | DAPIPRAZOLE | C19H27N5 | Dapiprazole | | ADRA1A |
| XMXHEBAFVSFQEX-UHFFFAOYSA-N | CHEMBL14376 | ILOPERIDONE | C24H27FN2O4 | Iloperidone; HP-873 | Fanapt; Iloperidone | ADRA2A |
| BQJCRHHNABKAKU-KBQPJGBKSA-N | CHEMBL70 | MORPHINE | C17H19NO3 | Morphine; Avinza; Depodur; | | ADRA2A |
| | | | | Infumorph; Kadian; IDS- | | |
| | | | | NM-009; N02AA01 | | |
| ULSDMUVEXKOYBU-ZDUSSCGKSA-N | CHEMBL1185 | ZOLMITRIPTAN | C16H21N3O2 | Zolmitriptan; 311C90; 311- | Zolmitriptan; Zomig; Zomig- | ADRA2A |
| | | | | C-90 | Zmt | |
| KSMAGQUYOIHWFS-UHFFFAOYSA-N | CHEMBL17860 | LOFEXIDINE | C11H12Cl2N2O | Lofexidine | | ADRA2A |
| KXSUAWAUCNFBQJ-UHFFFAOYSA-N | CHEMBL1255582 | FIPAMEZOLE | C14H15FN2 | Fipamezole | | ADRA2A |
| RTHCYVBBDHJXIQ-UHFFFAOYSA-N | CHEMBL41 | FLUOXETINE | C17H18F3NO | Fluoxetine; Prozac; Sarafem; | | ADRA2A |
| | | | | Symbyax; NSC-283480 | | |
| HSWPZIDYAHLZDD-UHFFFAOYSA-N | CHEMBL353972 | ATIPAMEZOLE | C14H16N2 | Atipamezole; MPV-1248 | Antisedan | ADRA2A |
| IEJXVRYNEISIKR-UHFFFAOYSA-N | CHEMBL647 | APRACLONIDINE | C9H10Cl2N4 | Apraclonidine | Iopidine; Iopidine 1% | ADRA2A |
| GEFQWZLICWMTKF-CDUCUWFYSA-N | CHEMBL677 | LEVONORDEFRIN | C9H13NO3 | Corbadrine; Levonordefrin; | Neo-Cobefrin | ADRA2A |
| | | | | Neo-Cobefrin; Nordefrin | | |
| HPMRFMKYPGXPEP-UHFFFAOYSA-N | CHEMBL10316 | IDAZOXAN | C11H12N2O2 | Idazoxan; RX-781094 | | ADRA2A |
| XYLJNLCSTIOKRM-UHFFFAOYSA-N | CHEMBL844 | BRIMONIDINE | C11H10BrN5 | Brimonidine; AGN-190342 | | ADRA2A |
| | | | | FREE BASE; UK-14304; UK- | | |
| | | | | 1430418 FREE BASE | | |
| WDZVGELJXXEGPV-YIXHJXPBSA-N | CHEMBL420 | GUANABENZ | C8H8Cl2N4 | Guanabenz; NSC- | | ADRA2A |
| | | | | 68982; WY-8678 | | |
| UCTWMZQNUQWSLP-VIFPVBQESA-N | CHEMBL679 | EPINEPHRINE | C9H13NO3 | Epinephrine; Adrenaline; | Adren | ADRA2A |
| | | | | Epinephrine HCl; Levo- | Co; Adrenaclick; Adrenalin; | |
| | | | | Methylaminoethanolcatechol; | Ana-Guard; Ana- | |
| | | | | Twinject; NSC-62786 | Kit; Anapen; Anaphylactic | |
| | | | | | Shock Drug Kit; Asma- | |
| | | | | | Vydrin; Asthmahaler; Auvi- | |
| | | | | | Q; Bronkaid | |
| | | | | | Mist; Brovon; Emerade; Epi | |
| | | | | | E Z Pen | |
| | | | | | Jr; Epinephrine; Epipen; Epipen | |
| | | | | | E Z Pen; Epipen Jr; Epipen | |
| | | | | | Jr .; Eppy; Gppe Inh | |
| | | | | | Soln; Jext; Medihaler- | |
| | | | | | epi; Primatene; Primatene | |
| | | | | | Mist; Riddobron; Riddofan; | |
| | | | | | Rybarvin; Simplene; Soln Made To | |
| | | | | | Rybarvin For; Sus-Phrine; Sus- | |
| | | | | | phrine sulfite free; Sus-Phrine | |
| | | | | | Sulfite-Free; Symjepi; Twinject | |
| | | | | | 0.15; Twinject 0.3 | |
| SFLSHLFXELFNJZ-QMMMGPOBSA-N | CHEMBL1437 | NOREPINEPHRINE | C8H11NO3 | Norepinephrine; Levophed; | | ADRA2A |
| | | | | R-norepinephrine | | |
| GJSURZIOUXUGAL-UHFFFAOYSA-N | CHEMBL134 | CLONIDINE | C9H9Cl2N3 | Clonidine; Catapres; Clorpres; | Catapres; Catapres-Tts- | ADRA2A; |
| | | | | Combipres; Duraclon; ST- | 1; Catapres-Tts-2; Catapres- | ADRA2A |
| | | | | 155-BS; ST-155BS | Tts-3; Clonidine | |
| GJSURZIOUXUGAL-UHFFFAOYSA-N | CHEMBL134 | CLONIDINE | C9H9Cl2N3 | Clonidine; Catapres; Clorpres; | Catapres; Catapres-Tts- | ADRA2A; |
| | | | | Combipres; Duraclon; ST- | 1; Catapres-Tts-2; Catapres- | ADRA2A |
| | | | | 155-BS; ST-155BS | Tts-3; Clonidine | |
| WZHJKEUHNJHDLS-QTGUNEKASA-N | CHEMBL19215 | METERGOLINE | C25H29N3O2 | Metergoline; FI-6337; MCE | Liserdol | ADRA2B |
| WIKYUJGCLQQFNW-UHFFFAOYSA-N | CHEMBL728 | PROCHLORPERAZINE | C20H24ClN3S | Prochlorperazine; | Compazine; Compro; | ADRA2B |
| | | | | Prochlorperazine Mesilate | Prochlorperazine; Stemetil Eff | |
| MJQMRGWYPNIERM-HNNXBMFYSA-N | CHEMBL2333941 | LY-2811376 | C15H14F2N4S | Ly- | | BACE1 |
| | | | | 2811376; Ly2811376; J3.079.793K; | | |
| | | | | LY2811376 | | |
| WKDNQONLGXOZRG-HRNNMHKYSA-N | CHEMBL3989948 | LANABECESTAT | 28N4C26HO | Lanabecestat; AZD- | | BACE1 |
| | | | | 3293; AZD3293; LY- | | |
| | | | | 3314814; LY3314814 | | |
| MRXBCEQZNKUUIP-DEOSSOPVSA-N | CHEMBL2177913 | AZD-3839 | C24H16F3N5 | Azd 3839; Azd- | | BACE1 |
| | | | | 3839; Azd3839; AZD3839 | | |
| YHYKUSGACIYRML-KRWDZBQOSA-N | CHEMBL3301601 | VERUBECESTAT | C17H17F2N5O3S | Verubecestat; MK- | | BACE1 |
| | | | | 8931; SCH 900931; SCH- | | |
| | | | | 900931; SCH900931 | | |
| GNMUEVRJHCWKTO-FQEVSTJZSA-N | CHEMBL3581647 | BIRABRESIB | C25H22ClN5O2S | Birabresib; MK-8628; OTX- | | BRD2 |
| | | | | 015; OTX-015 ANHYDROUS; OTX015 | | |
| | | | | ANHYDROUS | | |
| RDONXGFGWSSFMY-UHFFFAOYSA-N | CHEMBL3987016 | MIVEBRESIB | C22H19F2N3O4S | Mivebresib; Abbv- | | BRD2 |
| | | | | 075; ABBV-075; ABBV075 | | |
| WAEXFXRVDQXREF-UHFFFAOYSA-N | CHEMBL98 | VORINOSTAT | C14H20N2O3 | Vorinostat; MK- | Zolinza | BRD4 |
| | | | | 0683; MK0683 | | |
| CMSUJGUHYXQSOK-UHFFFAOYSA-N | CHEMBL3982690 | ALOBRESIB | 23N5C26HO2 | Alobresib; Gs-5829 | | BRD4 |
| RSMYFSPOTCDHHJ-GOSISDBHSA-N | CHEMBL4078100 | AZD-5153 | C25H33N7O3 | Azd 5153; Azd- | | BRD4 |
| | | | | 5153; Azd5153; AZD- | | |
| | | | | 5153; AZD5153 | | |
| FPOHNWQLNRZRFC-ZHACJKMWSA-N | CHEMBL483254 | PANOBINOSTAT | C21H23N3O2 | Panobinostat; LBH589 | Farydak | BRD4 |
| BZKPWHYZMXOIDC-UHFFFAOYSA-N | CHEMBL20 | ACETAZOLAMIDE | C4H6N4O3S2 | Acetazolamide; L | Acetazolamide; Diamox; | CA1 |
| | | | | 579486; NSC-145177 | Diamox SR; Eytazox | |
| HCRKCZRJWPKOAR-JTQLQIEISA-N | CHEMBL220491 | BRINZOLAMIDE | C12H21N3O5S3 | Brinzolamide; Azopt; AL- | Azopt | |
| | | | | 4862 | | |
| TYMRLRRVMHJFTF-UHFFFAOYSA-N | CHEMBL419 | MAFENIDE | C7H10N2O2S | Mafenide; 4- | Sulfamylon | CA12 |
| | | | | Homosulfanilamide; D06BA03; | | |
| | | | | NSC-34632 | | |
| SETFNECMODOHTO-UHFFFAOYSA-N | CHEMBL77517 | INDISULAM | C14H12ClN3O4S2 | Indisulam; E-7070; E7070 | | CA12 |
| NDDAHWYSQHTHNT-UHFFFAOYSA-N | CHEMBL406 | INDAPAMIDE | C16H16ClN3O3S | Indapamide; KYD-041; S- | Cardide SR; Ethibide | CA12 |
| | | | | 1520; SE-1520 | XL; Indapamide; Indaxa | |
| | | | | | 2.5; Indipam | |
| | | | | | XL; Lozol; Mapemid | |
| | | | | | XL; Natrilix; Natrilix SR; Nindaxa | |
| | | | | | 2.5; Opumide 2.5; Rawel | |
| | | | | | XL; Tensaid XL; Varbim XL | |
| AVSMSXHPIYIKIJ-UHFFFAOYSA-N | CHEMBL3087323 | DTP-348 | C6H11N5O4S | Dtp-348; Dtp348; DTP348 | | CA12 |
| KJADKKWYZYXHBB-XBWDGYHZSA-N | CHEMBL220492 | TOPIRAMATE | C12H21NO8S | Topiramate; MCN- | Epitomax; Qudexy | CA12 |
| | | | | 4853; RWJ-17021; RWJ- | XR; Topamax; Topamax | |
| | | | | 17021-000; USL- | Sprinkle; Topiramate; Trokendi | |
| | | | | 255; USL255 | XR | |
| BGRJTUBHPOOWDU-NSHDSACASA-N | CHEMBL267044 | LEVOSULPIRIDE | C15H23N3O4S | Levosulpiride; Sulpiride I- | | CA12 |
| | | | | form | | |
| IAVUPMFITXYVAF-XPUUQOCRSA-N | CHEMBL218490 | DORZOLAMIDE | C10H16N2O4S3 | Dorzolamide; Trusopt; MK- | Dorzant; Trusopt | CA12 |
| | | | | 507 | | |
| BGRJTUBHPOOWDU-UHFFFAOYSA-N | CHEMBL26 | SULPIRIDE | C15H23N304S | Sulpiride; N05AL01; RD- | Dogmatyl; Dolmatil | CA12 |
| | | | | 1403 | | |
| JIVPVXMEBJLZRO-UHFFFAOYSA-N | CHEMBL1055 | CHLORTHALIDONE | C14H11ClN204S | Chlortalidone; Chlorthalidone; | Chlorthalidone; Hygroton; | CA12 |
| | | | | G-33182; NSC-69200 | Thalitone | |
| FLOSMHQXBMRNHR-QPJJXVBHSA-N | CHEMBL19 | METHAZOLAMIDE | C5H8N4O3S2 | Methazolamide; L- | Methazolamide; Neptazane | CA12 |
| | | | | 584601; VVP-808; VVP808 | | |
| HHZIURLSWUIHRB-UHFFFAOYSA-N | CHEMBL255863 | NILOTINIB | C28H22F3N7O | Nilotinib; AMN- | Tasigna | CA2 |
| | | | | 107; AMN107 | | |
| OUZWUKMCLIBBOG-UHFFFAOYSA-N | CHEMBL18 | ETHOXZOLAMIDE | C9H1ON2O3S2 | Ethoxzolamide | Cardrase; Ethamide | CA2 |
| UBQNRHZMVUUOMG-UHFFFAOYSA-N | CHEMBL750 | ZONISAMIDE | C8H8N2O3S | Zonisamide; AD-810; CI- | Zonegran; Zonisamide | CA2 |
| | | | | 912; PD-110843 | | |
| GJQPMPFPNINLKP-UHFFFAOYSA-N | CHEMBL17 | DICHLORPHENAMIDE | C6H6Cl2N2O4S2 | Diclofenamide; Dichlorphenamide | Daranide; Keveyis; Oratrol | CA2 |
| HMHVCUVYZFYAJI-UHFFFAOYSA-N | CHEMBL328560 | SULTHIAME | C10H14N2O4S2 | Sultiame; Sulthiame; Riker | Conadil; Ospolot; Trolone | CA2 |
| | | | | 594; RIKER-594 | | |
| AQCHWTWZEMGIFD-UHFFFAOYSA-N | CHEMBL878 | METOLAZONE | C16H16ClN3O3S | Metolazone; SR-720-22 | Diulo; Metenix; Metolazone; | CA7 |
| | | | | | Mykrox; Xuret; Zaroxolyn | |
| LMJSLTNSBFUCMU-UHFFFAOYSA-N | CHEMBL1054 | TRICHLORMETHIAZIDE | C8H8Cl3N3O4S2 | Trichlormethiazide | Metahydrin; Naqua; Trichlorex; | CA7 |
| | | | | | Trichlormas; Trichlormethiazide | |
| CVHZOJJKTDOEJC-UHFFFAOYSA-N | CHEMBL310671 | SACCHARIN | C7H5NO3S | E954; Saccharin; Benzosulfinide; | Sweeta | CA7 |
| | | | | Gluside; E-954(1); INS | | |
| | | | | NO.954(1); INS-954(1); NSC- | | |
| | | | | 5349 | | |
| IQPNAANSBPBGFQ-UHFFFAOYSA-N | CHEMBL151 | LUTEOLIN | C15H10O6 | Luteolin; Digitoflavone; | | CA7 |
| | | | | Flacitran; Luteoline; Luteolol | | |
| OVSQVDMCBVZWGM-QSOFNFLRSA- N | CHEMBL250450 | ISOQUERCETIN | C21H20O12 | Isoquercitrin; Isoquercetin; | | CA7 |
| | | | | Quercetin 3-o- | | |
| | | | | glucopyranoside; NSC- | | |
| | | | | 115918; NSC-407304 | | |
| ZQSIJRDFPHDXIC-UHFFFAOYSA-N | CHEMBL8145 | DAIDZEIN | C15H10O4 | Daidzein | | CA7 |
| XUFQPHANEAPEMJ-UHFFFAOYSA-N | CHEMBL902 | FAMOTIDINE | C8H15N7O2S3 | Famotidine; L 643341; MK- | Famotidine; Famotidine | CA7 |
| | | | | 208; YM-11170 | preservative | |
| | | | | | free; Fluxid; Pepcid; Pepcid | |
| | | | | | Ac; Pepcid preservative | |
| | | | | | free; Pepcid Rpd | |
| WJRRGYBTGDJBFX-UHFFFAOYSA-N | CHEMBL488436 | AZD-5438 | C18H21N5O2S | AZD-5438 | | CDK2 |
| PDGKHKMBHVFCMG-UHFFFAOYSA-N | CHEMBL3894860 | TRILACICLIB | C24H30N8O | Trilaciclib; G1T28 | | CDK2 |
| XLSYZSRXVVCHLS-UHFFFAOYSA-N | CHEMBL3545083 | RGB-286638 | C29H35N7O4 | RGB-286638; RGB 286638 | | CDK2 |
| VXBAJLGYBMTJCY-NSCUHMNNSA-N | CHEMBL1944698 | ZOTIRACICLIB | C23H24N4O | TG-02; Tg02; EX45; SB- | | CDK2 |
| | | | | 1317; SB1317; TG02; | | |
| | | | | Zotiraciclib | | |
| YPJRHEKCFKOVRT-UHFFFAOYSA-N | CHEMBL3904602 | LEROCICLIB | C26H34N8O | Lerociclib; G1t38; G1T38; | | CDK2 |
| | | | | G1T38 FREE BASE | | |
| HUXYBQXJVXOMKX-UHFFFAOYSA-N | CHEMBL1230607 | PHA-793887 | C19H31N5O2 | PHA-793887 | | CDK2 |
| AHJRHEGDXFFMBM-UHFFFAOYSA-N | CHEMBL189963 | PALBOCICLIB | 29N7C24HO2 | Palbociclib; PD-0332991 | Ibrance | CDK2 |
| LGLFFNDHMLKUMI-UHFFFAOYSA-N | CHEMBL459265 | HEXAMETHYL | C25H30N3+ | | | CDK2 |
| | | PARAROSANILINE | | | | |
| JRNJNYBQQYBCLE-UHFFFAOYSA-N | CHEMBL384304 | RG-547 | C18H21F2N5O4S | R-547; RG-547; | | CDK2 |
| | | | | Ro4584820; R547; | | |
| | | | | RO4584820 | | |
| PIMQWRZWLQKKBJ-SFHVURJKSA-N | CHEMBL2103840 | DINACICLIB | C21H28N6O2 | Dinaciclib; MK-7965; SCH- | | CDK2 |
| | | | | 727965 | | |
| CXQHYVUVSFXTMY-UHFFFAOYSA-N | CHEMBL1230609 | FORETINIB | C34H34F2N4O6 | Foretinib; GSK1363089; EXE | | CDK7 |
| | | | | L-2880; GSK- | | |
| | | | | 1363089G; GSK089; GSK136 | | |
| | | | | 3089G; XL-880; XL880 | | |
| ODPGGGTTYSGTGO-UHFFFAOYSA-N | CHEMBL574738 | AST-487 | C26H30F3N7O2 | AST-487; NVP-AST487 | | CDK7 |
| OVPNQJVDAFNBDN-UHFFFAOYSA-N | CHEMBL445813 | AT-7519 | C16H17Cl2N5O2 | AT-7519; AT 7519; AT7519 | | CDK7 |
| IAYGCINLNONXHY-LBPRGKRZSA-N | CHEMBL2041933 | AZD-7762 | C17H19FN4O2S | Azd 7762; AZD- | | CHEK2 |
| | | | | 7762; AZD7762 | | |
| PBCZSGKMGDDXIJ-KRUBCLEUSA-N | CHEMBL574737 | UCN-01 | C28H26N4O4 | Ucn 01; UCN-01; KRX- | | CHEK2 |
| | | | | 0601; KW-2401; NSC- | | |
| | | | | 638850; UCN-02 | | |
| WINHZLLDWRZWRT-ATVHPVEESA-N | CHEMBL535 | ISUNITINIB | C22H27FN4O2 | Sunitinib; Sutent; SU- | Sutent | CHEK2 |
| | | | | 011248; SU- 11248; SU011248 | | |
| MUOKSQABCJCOPU-UHFFFAOYSA-N | CHEMBL1230165 | SILMITASERTIB | C19H12ClN3O2 | Silmitasertib; CX-4945; CX | | CLK2 |
| | | | | 4945; CX4945 | | |
| UIARLYUEJFELEN-LROUJFHJSA-N | CHEMBL603469 | LESTAURTINIB | C26H21N3O4 | Lestaurtinib; Cep-701; A- | | CLK4 |
| | | | | 154475; A-1544750; CEP- | | |
| | | | | 701; KT-555; KT-5555; SP- | | |
| | | | | 924; SP924; SPM-924 | | |
| BNVPFDRNGHMRJS-UHFFFAOYSA-N | CHEMBL3674570 | EDICOTINIB | 35N5C27HO2 | Edicotinib; JNJ-40346527 | | CSF1R |
| WPHKIQPVPYJNAX-UHFFFAOYSA-N | CHEMBL1980297 | ILORASERTIB | C25H21FN6O2S | Ilorasertib; ABT-348; A- | ABT-348, A-968660 | CSF1R |
| | | | | 968660; A- | | |
| | | | | 968660.0; ABBOTT-968660 | | |
| JGWRKYUXBBNENE-UHFFFAOYSA-N | CHEMBL3813873 | PEXIDARTINIB | C20H15ClF3N5 | Pexidartinib; PLX- | | CSF1R |
| | | | | 3397; CML-261; PLX3397 | | |
| DZFZXPPHBWCXPQ-UHFFFAOYSA-N | CHEMBL2180604 | TAK-593 | C23H23N7O3 | TAK-593; TAK 593 | | CSF1R |
| CUIHSIWYWATEQL-UHFFFAOYSA-N | CHEMBL477772 | PAZOPANIB | 23N7C21HO2S | Pazopanib; GW786034 | Votrient | CSF1R |
| UXXQOJXBIDBUAC-UHFFFAOYSA-N | CHEMBL124660 | TANDUTINIB | C31H42N6O4 | Tandutinib; MLN-0518; CT- | | CSF1R |
| | | | | 53518; CT53518; MLN-518 | | |
| PIQCTGMSNWUMAF-UHFFFAOYSA-N | CHEMBL522892 | DOVITINIB | C21H21FN6O | Dovitinib; TKI-258; CHIR- | | CSF1R |
| | | | | 258; GFKI-258; NVP-TKI258 | | |
| DKNUPRMJNUQNHR-UHFFFAOYSA-N | CHEMBL2029988 | CEP-32496 | C24H22F3N5O5 | Agerafenib; CEP- | | CSF1R |
| | | | | 32496; Rxdx 105; AB- | | |
| | | | | 024; AC-013773; RXDX-105 | | |
| CVWXJKQAOSCOAB-UHFFFAOYSA-N | CHEMBL576982 | QUIZARTINIB | C29H32N6O4S | Quizartinib; AC-220; AC- | | CSF1R |
| | | | | 010220; AC010220; AC220; ASP-2689 | | |
| MPVGZUGXCQEXTM-UHFFFAOYSA-N | CHEMBL223360 | LINIFANIB | C21H18FN5O | Linifanib; A-741439; ABT- | | CSF1R |
| | | | | 869; AL-39324; RG-3635 | | |
| RAHBGWKEPAQNFF-UHFFFAOYSA-N | CHEMBL572881 | MOTESANIB | C22H23N5O | Motesanib; AMG-706 | | CSF1R |
| WJEOLQLKVOPQFV-UHFFFAOYSA-N | CHEMBL1908391 | MASITINIB | C28H30N6OS | Masitinib; AB-1010 | Kinavet; Masivet | CSF1R |
| ZBNZXTGUTAYRHI-UHFFFAOYSA-N | CHEMBL1421 | DASATINIB | C22H26ClN702S | Dasatinib; Dasatinib | Dasatinib; Sprycel | CSF1R |
| | | | | hydrate; Dasatinib | | |
| | | | | monohydrate; BMS- | | |
| | | | | 354825; BMS-354825 | | |
| | | | | HYDRATE; BMS-354825-03 | | |
| KTUFNOKKBVMGRW-UHFFF AOYSA-N | CHEMBL941 | IMATINIB | 31N7C29HO | Imatinib; Gleevec; STI-571 | | CSF1R |
| CTNPALGJUAXMMC-PMFHANACSA-N | CHEMBL1721885 | SU-014813 | C23H27FN4O4 | SU-014813; Su-14813; SU- | | CSF1R |
| | | | | 14813 | | |
| BWYBBMQLUKXECQ-GIVPXCGWSA-N | CHEMBL203665 | RELACATIB | C27H32N4O6S | Relacatib; SB-462795 | | CTSK |
| LLCRBOWRJOUJAE-UHFFFAOYSA-N | CHEMBL371064 | BALICATIB | C23H33N5O2 | Balicatib; AAE-581; AAE581 | | CTSK |
| FWIVDMJALNEADT-SFTDATJTSA-N | CHEMBL481611 | ODANACATIB | C25H27F4N3O3S | Odanacatib; MK- | | CTSK |
| | | | | 0822; MK0822 | | |
| KFQYTPMOWPVWEJ-INIZCTEOSA-N | CHEMBL1303 | ROTIGOTINE | C19H25NOS | Rotigotine; SPM-962 | Neupro | DRD2 |
| BVPWJMCABCPUQY-UHFFFAOYSA-N | CHEMBL325109 | CLEBOPRIDE | C20H24ClN3O2 | Clebopride; Clebopride | Clanzoflat; Cleboril | DRD2 |
| | | | | Malate; LAS-9273 | | |
| WAOQONBSWFLFPE-VIFPVBQESA-N | CHEMBL8809 | RACLOPRIDE | C15H20Cl2N2O3 | Raclopride | | DRD2 |
| RKZSNTNMEFVBDT-MRVPVSSYSA-N | CHEMBL419792 | SUMANIROLE | C11H13N3O | Sumanirole; PNU-95666E | | DRD2 |
| FASDKYOPVNHBLU-ZETCQYMHSA-N | CHEMBL301265 | PRAMIPEXOLE | 17N3C10HS | Pramipexole; SUD- | Mirapexin; Neliprax; Oprymea; | DRD2 |
| | | | | 919CL2Y; U-98528E | Pipexus | |
| QGNOXTFZOLDODX-UHFFFAOYSA-N | CHEMBL1774080 | PF-00217830 | C26H30N4O2 | Pf- | | DRD2 |
| | | | | 00217830; J2.975.999E; PF- | | |
| | | | | 00217830; PF-217830 | | |
| PMXMIIMHBWHSKN-UHFFFAOYSA-N | CHEMBL1621 | PALIPERIDONE | C23H27FN4O3 | Paliperidone; R-76477; Ro- | Invega; Paliperidone; Trevicta; | DRD2 |
| | | | | 76477 | Xeplion | |
| FEBOTPHFXYHVPL-UHFFFAOYSA-N | CHEMBL297302 | BENPERIDOL | C22H24FN3O2 | Benperidol; MCN-JR- | Anquil; Benquil | DRD2 |
| | | | | 4584; NSC-170982; R-4584 | | |
| ZKIAIYBUSXZPLP-UHFFFAOYSA-N | CHEMBL2105760 | BREXPIPRAZOLE | C25H27N3O2S | Brexpiprazole; OPC-34712 | Rexulti | DRD2 |
| CYGODHVAJQTCBG-UHFFFAOYSA-N | CHEMBL218166 | BIFEPRUNOX | C24H23N3O2 | Bifeprunox | | DRD2 |
| GUTXTARXLVFHDK-UHFFFAOYSA-N | CHEMBL1200986 | HALOPERIDOL | C31H41ClFNO3 | Haloperidol Decanoate; R- | Haldol; Haldol | DRD2 |
| | | DECANOATE | | 13672 | Dec; Haloperidol Decanoate | |
| YFGHCGITMMYXAQ-LJQANCHMSA-N | CHEMBL1201192 | ARMODAFINIL | C15H15NO2S | Armodafinil; CEP- | Armodafinil; Nuvigil | DRD2 |
| | | | | 10952; CEP-10953; CRL- | | |
| | | | | 40982 | | |
| YFGHCGITMMYXAQ-UHFFFAOYSA-N | CHEMBL1373 | MODAFINIL | C15H15NO2S | Modafinil; CEP-1538; CRC- | Modafinil; Provigil | DRD2 |
| | | | | 40476; CRL-40476; DEP- | | |
| | | | | 1538; THN-102 | | |
| | | | | COMPONENT | | |
| | | | | MODAFINIL; THN102 | | |
| | | | | COMPONENT | | |
| | | | | MODAFINIL; Modafinil civ | | |
| SLVMESMUVMCQIY-UHFFFAOYSA-N | CHEMBL1088 | MESORIDAZINE | C21H26N2OS2 | Mesoridazine; Serentil; NC- | Lidanar | DRD2 |
| | | | | 123; TPS-23 | | |
| VSWBSWWIRNCQIJ-UHFFFAOYSA-N | CHEMBL3187365 | ASENAPINE | C17H16ClNO | Asenapine | Sycrest | DRD2 |
| XVGOZDAJGBALKS-UHFFFAOYSA-N | CHEMBL178803 | BLONANSERIN | C23H30FN3 | Blonanserin; AD-5423 | | DRD2 |
| KAAZGXDPUNNEFN-UHFFFAOYSA-N | CHEMBL304902 | CLOTHIAPINE | C18H18ClN3S | Clotiapine; Clothiapine; HF- | | DRD2 |
| | | | | 2159 | | |
| OABRYNHZQBZDMG-INIZCTEOSA-N | CHEMBL392158 | FALLYPRIDE | C20H29FN2O3 | Fallypride | | DRD2 |
| UHSKFQJFRQCDBE-UHFFFAOYSA-N | MBLCHE589 | ROPINIROLE | C16H24N2O | Ropinirole; Requip; SK&F- | Requip | DRD2 |
| | | | | 101468 | | |
| HKFMQJUJWSFOLY-OAQYLSRUSA-N | CHEMBL220808 | SARIZOTAN | C22H21FN2O | Sarizotan; EMD-77697 | | DRD2 |
| RGCVKNLCSQQDEP-UHFFFAOYSA-N | CHEMBL567 | PERPHENAZINE | C21H26ClN3OS | Perphenazine; Perphenazine | Chlorpiprazine; Decentan; | DRD2 |
| | | | | Fendizoate; Perphenazine | Fentazin; Perphenazine; | |
| | | | | Maleate; Etaperazine; NSC- | Perphenazine Fendizoate; | |
| | | | | 150866 | Trilafon; Trilafon-La | |
| VYFYYTLLBUKUHU-UHFFFAOYSA-N | CHEMBL59 | DOPAMINE | C8H11NO2 | Dopamine; Carbilev; Intropin; | | DRD2 |
| | | | | Parcopa; Sinemet; NSC-173182 | | |
| VMWNQDUVQKEIOC-CYBMUJFWSA-N | CHEMBL53 | APOMORPHINE | C17H17NO2 | Apomorphine; APL- | | DRD2 |
| | | | | 130277; VR-040; VR040 | | |
| YVUQSNJEYSNKRX-UHFFFAOYSA-N | CHEMBL1423 | PIMOZIDE | C28H29F2N3O | Pimozide; MCN-JR- | Orap; Pimozide | DRD2 |
| | | | | 6238; NSC-170984; R 623; R- | | |
| | | | | 623; R-6238 | | |
| CEUORZQYGODEFX-UHFFFAOYSA-N | CHEMBL1112 | ARIPIPRAZOLE | C23H27Cl2N3O2 | Aripiprazole; OPC- | Abilify; Abilify | DRD2 |
| | | | | 14597; OPC-31 | Maintena; Abilify maintena | |
| | | | | | kit; Abilify mycite | |
| | | | | | kit; Aripiprazole | |
| JUQLTPCYUFPYKE-UHFFFAOYSA-N | CHEMBL267777 | RITANSERIN | C27H25F2N3OS | Ritanserin; R-55667 | Tiserton | DRD2 |
| WSPOMRSOLSGNFJ-AUWJEWJLSA-N | CHEMBL908 | CHLORPROTHIXENE | C18H18ClNS | Chlorprothixene; N-714; N- | Taractan; Truxal | DRD2 |
| | | | | 714RO 4-0403; NSC- | | |
| | | | | 18720; RO-4-0403; Ro- | | |
| | | | | 40403 | | |
| NJMYODHXAKYRHW-BLLMUTORSA-N | CHEMBL42055 | E-FLUPENTIXOL | C23H25F3N2OS | E-flupentixol; | | DRD2 |
| | | | | Flupenthixol; | | |
| | | | | FIupentixole | | |
| ZEWQUBUPAILYHI-UHFFFAOYSA-N | CHEMBL422 | TRIFLUOPERAZINE | C21H24F3N3S | Trifluoperazine | Amylozine; Stelazine; Stelazine | DRD2 |
| | | | | | Fte | |
| FGXWKSZFVQUSTL-UHFFFAOYSA-N | CHEMBL219916 | DOMPERIDONE | C22H24ClN5O2 | Domperidone; R-33812 | Domilium; Evoxin; Motilium; | DRD2 |
| | | | | | Motilium 10; Motilium | |
| | | | | | Instants; Vivadone | |
| IUVSEUFHPNITEQ-UHFFFAOYSA-N | CHEMBL221692 | Adoprazine | C24H24FN3O2 | Adoprazine; SLV-313 | | DRD2 |
| NTJOBXMMWNYJFB-UHFFFAOYSA-N | CHEMBL243712 | AMISULPRIDE | C17H27N3O4S | Amisulpride; APD- | Solian; Solian 100; Solian | DRD2 |
| | | | | 421; APD421; DAN-2163 | 200; Solian 400; Solian 50 | |
| YEHCICAEULNIGD-MZMPZRCHSA-N | CHEMBL531 | PERGOLIDE | 26N2C19HS | Pergolide | | DRD2 |
| KPWSJANDNDDRMB-QAQDUYKDSA-N | CHEMBL2028019 | CARIPRAZINE | C21H32Cl2N4O | Cariprazine; GED-129; MP- | Reagila | DRD2 |
| | | | | 214; RGH-188 | | |
| NJMYODHXAKYRHW-DVZOWYKESA-N | CHEMBL54661 | FLUPENTIXOL | C23H25F3N2OS | Flupentixol; LC-44; N-7009 | | DRD2 |
| CYQFCXCEBYINGO-IAGOWNOFSA-N | CHEMBL465 | DRONABINOL | C21H30O2 | Dronabinol; delta-9- | Dronabinol; Marinol; Syndros | DRD3 |
| | | | | Tetrahydrocannabinol; | | |
| | | | | delta-9-THC; ABBOTT | | |
| | | | | 40566; ABBOTT- | | |
| | | | | 40566; J882F; NSC- | | |
| | | | | 134454; QCD 84924; QCD- | | |
| | | | | 84924; SP 104; SP-104 | | |
| YNNUSGIPVFPVBX-NHCUHLMSSA-N | CHEMBL1626 | CLEMASTINE | C21H26ClNO | Clemastine; HS-592 | | DRD3 |
| FPCCSQOGAWCVBH-UHFFFAOYSA-N | CHEMBL51 | KETANSERIN | C22H22FN3O3 | Ketanserin; R-41468 | Sufrexal | DRD4 |
| XJGVXQDUIWGIRW-UHFFFAOYSA-N | CHEMBL831 | LOXAPINE | C18H18ClN3O | Loxapine; Loxapac; Loxitane; | Adasuve; Loxitane | DRD4 |
| | | | | CL-62362; SUM 3170; SUM-3170 | Intramuscular [as | |
| | | | | | hydrochloride]; Loxitane-C | |
| | | | | | Oral Suspension [as | |
| | | | | | hydrochloride] | |
| DMJWENQHWZZWDF-PKOBYXMFSA-N | CHEMBL298406 | ECOPIPAM | C19H20ClNO | Ecopipam; SCH-39166 | | DRD5 |
| GZUITABIAKMVPG-UHFFFAOYSA-N | CHEMBL81 | RALOXIFENE | C28H27NO4S | Raloxifene; Evista; J22.982B; | | EBP |
| | | | | LY-139481 | | |
| NKANXQFJJICGDU-QPLCGJKRSA-N | CHEMBL83 | TAMOXIFEN | C26H29NO | Tamoxifen; Nolvadex; Soltamox; | | EBP |
| | | | | ICI-46474; ICI-47699 | | |
| GKIRPKYJQBWNGO-UHFFFAOYSA-N | CHEMBL 2355051 | CLOMIPHENE | C26H28ClNO | Clomifene; Clomiphene | | EBP |
| GKIRPKYJQBWNGO-OCEACIFDSA-N | CHEMBL954 | ENCLOMIPHENE | C26H28ClNO | Enclomifene; Cisclomiphene; | | EBP |
| | | | | Enclomiphene; Isomer | | |
| | | | | B; Milophene; Serophene; | | |
| | | | | RMI-16289 | | |
| UYNVMODNBIQBMV-UHFFFAOYSA-N | CHEMBL305187 | IFENPRODIL | C21H27NO2 | Ifenprodil; Ifenprodil | Vadilex | EBP |
| | | | | Tartrate; RC 61-91; RC-61- | | |
| | | | | 91 | | |
| SYHDSBBKRLVLFF-UHFFFAOYSA-N | CHEMBL187709 | TRIPARANOL | C27H32ClNO2 | Triparanol | | EBP |
| HVXKQKFEHMGHSL-QKDCVEJESA-N | CHEMBL3544983 | TESEVATINIB | C24H25Cl2FN4O2 | Tesevatinib; XL-647; EXEL- | | EGFR |
| | | | | 7647; KD-019; KD- | | |
| | | | | 020; KD019; XL647 | | |
| HUFOZJXAKZVRNJ-UHFFFAOYSA-N | CHEMBL3545308 | ROCILETINIB | C27H28F3N7O3 | Rociletinib; AVL-301; CNX- | | EGFR |
| | | | | 419; CO-1686; CS-1631 | | |
| LLVZBTWPGQVVLW-SNAWJCMRSA-N | CHEMBL483321 | CP-724714 | C27H27N5O3 | CP-724714; CP-724,714 | | EGFR |
| OONFNUWBHFSNBT-HXUWFJFHSA-N | CHEMBL587723 | AEE-788 | C27H32N6 | 5343; NVP-AEE-788; NVP- | | EGFR |
| | | | | Aee 788; AEE- | | |
| | | | | 788; AEE788; GNF-PF- | | |
| | | | | AEE788 | | |
| PLIVFNIUGLLCEK-UHFFFAOYSA-N | CHEMBL598797 | CUDC-101 | C24H26N4O4 | Cudc 101; CUDC-101 | | EGFR |
| FTFRZXFNZVCRSK-UHFFFAOYSA-N | CHEMBL258940 | FALNIDAMOL | C18H19ClFN7 | Falnidamol; Bibx | | EGFR |
| | | | | 1382; BIBX-1382; BIBX 1382 | | |
| MXDSJQHFFDGFDK-CYBMUJFWSA-N | CHEMBL3623290 | AZD-3759 | C22H23ClFN5O | Azd 3759; Azd- | | |
| | | | | 3759; Azd3759; AZD3759 | | EGFR |
| ZDZOTLJHXYCWBA-VCVYQWHSSA-N | CHEMBL92 | DOCETAXEL | C43H53NO14 | Taxotere; RP-56976 | | EGFR |
| DFJSJLGUIXFDJP-UHFFFAOYSA-N | CHEMBL2408045 | SAPITINIB | C23H25ClFN5O3 | Sapitinib; AZD- | | EGFR |
| | | | | 8931; AZD8931 | | |
| QKDCLUARMDUUKN-IXPASAXMMP-N | CHEMBL3663929 | NAQUOTINIB | C30H42N8O3 | Naquotinib; ASP- | | EGFR |
| | | | | 8273; ASP8273 | | |
| IOMMMLWIABWRKL-WUTDNEBXSA-N | CHEMBL3787344 | NAZARTINIB | C26H31ClN6O2 | Nazartinib; EGF-816; NVP- | | EGFR |
| | | | | EGF816-NX | | |
| FDMQDKQUTRLUBU-UHFFFAOYSA-N | CHEMBL3786343 | OLMUTINIB | C26H26N6O2S | Olmutinib; HM-61713; BI | | EGFR |
| | | | | 1482694; BI- | | |
| | | | | 1482694; HM61713 | | |
| JYIUNVOCEFIUIU-GHMZBOCLSA-N | CHEMBL3989970 | MAVELERTINIB | C18H22FN9O2 | Mavelertinib; PF-06747775 | | EGFR |
| ODMXWZROLKITMS-RISCZKNCSA-N | CHEMBL3786098 | PF-06459988 | C19H22ClN7O3 | Pf-06459988; PF-06459988 | | EGFR |
| LVXJQMNHJWSHET-AATRI KPKSA-N | CHEMBL2110732 | DACOMITINIB | C24H25ClFN5O2 | Dacomitinib; Dacomitinib | | EGFR |
| | | | | hydrate; PF-00299804 | | |
| XGALLCVXEZPNRQ-UHFFFAOYSA-N | CHEMBL939 | GEFITINIB | C22H24ClFN4O3 | Gefitinib; Iressa; ZD-1839 | Iressa | EGFR |
| QQLKULDARVNMAL-UHFFFAOYSA-N | CHEMBL2087361 | ICOTINIB | C22H21N3O4 | Icotinib; BPI-2009H | | EGFR |
| JWNPDZNEKVCWMY-VQHVLOKHSA-N | CHEMBL180022 | NERATINIB | C30H29ClN6O3 | Neratinib; CDP-820; HKI- | | EGFR |
| | | | | 272; WAY-179272 | | |
| ZYQXEVJIFYIBHZ-UHFFFAOYSA-N | CHEMBL1614725 | TAK-285 | C26H25ClF3N5O3 | Tak 285; TAK-285 | | EGFR |
| BMGQWWVMWDBQGC-IIFHNQTCSA-N | CHEMBL608533 | MIDOSTAURIN | C35H30N4O4 | Midostaurin; PKC-412; CGP- | Rydapt | EGFR |
| | | | | 41251; NVP-PKC412 | | |
| ULXXDDBFHOBEHA-CWDCEQMOSA-N | CHEMBL1173655 | AFATINIB | C24H25ClFN5O3 | Afatinib; BIBW-2992 | Gilotrif; Giotrif | EGFR |
| UHTHHESEBZOYNR-UHFFFAOYSA-N | CHEMBL24828 | VANDETANIB | C22H24BrFN4O2 | Vandetanib; GNF-PF- | Caprelsa; Zactima | EGFR |
| | | | | 2188; ZD-64; ZD-6474 | | |
| BCFGMOOMADDAQU-UHFFFAOYSA-N | CHEMBL554 | LAPATINIB | C29H26ClFN4O4S | Lapatinib; GSK- | Tyverb | EGFR |
| | | | | 572016; GW-2016; GW- | | |
| | | | | 572016; GW- | | |
| | | | | 572016X; GW572016 | | |
| AAKJLRGGTJKAMG-UHFFFAOYSA-N | CHEMBL553 | ERLOTINIB | C22H23N3O4 | Erlotinib; CP-358,774; CP- | Tarceva | EGFR |
| | | | | 358774; CP-35877401; OSI- | | |
| | | | | 774; R-1415; RG-1415; Ro- | | |
| | | | | 508231 | | |
| OMZCMEYTWSXEPZ-UHFFFAOYSA-N | CHEMBL31965 | CANERTINIB | C24H25ClFN5O3 | Canertinib; CI-1033 | | EGFR |
| XYFPWWZEPKGCCK-GOSISDBHSA-N | CHEMBL1873475 | IBRUTINIB | C25H24N6O2 | Ibrutinib; CRA-032765; PC- | Imbruvica | EGFR |
| | | | | 32765; PCI 32765; PCI- | | |
| | | | | 32765; PCI-32765-00 | | |
| LPFWVDIFUFFKJU-UHFFFAOYSA-N | CHEMBL3545154 | POZIOTINIB | C23H21Cl2FN4O3 | Poziotinib; HM-781- | | EGFR |
| | | | | 36B; HM 781-36B; NOV- | | |
| | | | | 120101 | | |
| UWXSAYUXVSFDBQ-CYBMUJFWSA-N | CHEMBL2103842 | VARLITINIB | C22H19ClN6O2S | Varlitinib; AR- | | EGFR |
| | | | | 00334543; ARRY- | | |
| | | | | 334543; ARRY-543; ASLAN- | | |
| | | | | 001; ASLAN001 | | |
| DUYJMQONPNNFPI-UHFFFAOYSA-N | CHEMBL335341 | OSIMERTINIB | C28H33N7O2 | Osimertinib; AZD- | Tagrisso | EGFR |
| | | | | 9291; AZD-9291 FREE BASE | | |
| WVUNYSQLFKLYNI-AATRIKPKSA-N | CHEMBL607707 | PELITINIB | C24H23ClFN5O2 | Pelitinib; EKB-569; WAY- | | EGFR |
| | | | | EKB-569 | | |
| MLDQJTXFUGDVEO-UHFFFAOYSA-N | CHEMBL1336 | SORAFENIB | C21H16ClF3N4O3 | Sorafenib; BAY-43-9006 | Nexavar | EGFR |
| UBPYILGKFZZVDX-UHFFFAOYSA-N | CHEMBL288441 | BOSUTINIB | C26H29Cl2N5O3 | Bosutinib; Bosutinib | Bosulif | EPHA8 |
| | | | | Monohydrate; SK-606; SKI- | | |
| | | | | 606 | | |
| BATCTBJIJJEPHM-UHFFFAOYSA-N | CHEMBL1651534 | PF-04457845 | C23H20F3N5O2 | Pf-04457845; PF-04457845 | | FAAH |
| WRBFPGYDKJBYBI-SJORKVTESA-N | CHEMBL2386558 | MK-3168 | C21H21ClN4OS | | | FAAH |
| AHLBNYSZXLDEJQ-FWEHEUNISA-N | CHEMBL175247 | ORLISTAT | C29H53NO5 | Orlistat; Lipstatin; Orlipastat; | ALLI; Beacita; Xenical | FAAH |
| | | | | Tetrahydrolipstatin; RO- | | |
| | | | | 180647-002; Ro- | | |
| | | | | 180647002 | | |
| ROFVXGGUISEHAM-UHFFFAOYSA-N | CHEMBL184238 | URB-597 | C20H22N2O3 | Urb-597; URB597; KDS- | | FAAH |
| | | | | 4103; ORG-231295; URB 597 | | |
| LOLPPWBBNUVNQZ-UHFFFAOYSA-N | CHEMBL495727 | AT-9283 | C19H23N7O2 | At 9283; AT-9283; AT9283 | | FLT1 |
| KGSRYTUWXUESJK-FXBPSFAMSA-N | CHEMBL2180602 | TAFETINIB | C24H29FN4O2 | Tafetinib; SIM 010603; SIM- | | FLT1 |
| | | | | 010603 | | |
| AEULIVPVIDOLIN-UHFFFAOYSA-N | CHEMBL2010872 | CEP-11981 | C28H27N7O | CEP-11981; BOL-303213X | | FLT1 |
| CUDVHEFYRIWYQD-UHFFFAOYSA-N | CHEMBL2220486 | LUCITANIB | C26H25N3O4 | Lucitanib; AL-3810; CO- | | FLT1 |
| | | | | 3810; E-3810; S-80881 | | |
| VPBYZLCHOKSGRX-UHFFFAOYSA-N | CHEMBL406381 | KRN-633 | C20H21ClN4O4 | KRN-633 | | FLT1 |
| JGEBLDKNWBUGRZ-HXUWFJFHSA-N | CHEMBL1822792 | MK2461 | C24H25N5O5S | MK-2461 | | FLT1 |
| SPMVMDHWKHCIDT-UHFFFAOYSA-N | CHEMBL1289494 | TIVOZANIB | C22H19ClN4O5 | Tivozanib; KRN-951; ASP- | Fotivda | FLT1 |
| | | | | 4130; AV-951; Kil- | | |
| | | | | 8951; Kil8951 | | |
| WUWDLXZGHZSWQZ-WQLSENKSSA-N | CHEMBL276711 | SEMAXANIB | C15H14N2O | Semaxanib; Semaxinib; SU- | | FLT1 |
| | | | | 5416; NSC- 696819; SU005416 | | |
| RITAVMQDGBJQJZ-FMIVXFBMSA-N | CHEMBL1289926 | AXITINIB | C22H18N4OS | Axitinib; AG-013736; AG- | Inlyta | FLT1 |
| | | | | 13736 | | |
| WCWUXEGQKLTGDX-LLVKDONJSA-N | CHEMBL377300 | BRIVANIB | C19H19FN4O3 | Brivanib; BMS-540215 | | FLT1 |
| YCOYDOIWSSHVCK-UHFFFAOYSA-N | CHEMBL101253 | VATALANIB | C20H15ClN4 | Vatalanib; PTK-787; BAY-86- | | FLT1 |
| | | | | 5127; CGP-79787; K- | | |
| | | | | 222584; NVP- | | |
| | | | | PTK787; PTK787; ZK-222584 | | |
| XXJWYDDUDKYVKI-UHFFFAOYSA-N | CHEMBL491473 | CEDIRANIB | C25H27FN4O3 | Cediranib; AZD-2171; ZD- | Recentin | FLT1 |
| | | | | 2171 | | |
| AILRADAXUVEEIR-UHFFFAOYSA-N | CHEMBL3545311 | BRIGATINIB | C29H39ClN7O2P | Brigatinib; AP-26113 | Alunbrig | FLT3 |
| MLIFNJABMANKEU-UHFFFAOYSA-N | CHEMBL150894 | CEP-5214 | C28H28N2O3 | CEP-5214 | | FLT3 |
| KXMZDGSRSGHMMK-VWLOTQADSA-N | CHEMBL3809489 | BEMCENTINIB | C30H34N8 | Bemcentinib; R-428; BGB | | FLT3 |
| | | | | 324; BGB-324; CS-1046; HY- | | |
| | | | | 15150; KB-80319; QC- | | |
| | | | | 11751; SYN-1131; W-5845 | | |
| ONIQOQHATWINJY-UHFFFAOYSA-N | CHEMBL2105717 | CABOZANTINIB | C28H24FN3O5 | Cabozantinib; BMS- | Cabometyx; Cometriq | FLT3 |
| | | | | 907351; XL-184; XL-184 | | |
| | | | | FREE BASE | | |
| DYNHJHQFHQTFTP-UHFFFAOYSA-N | CHEMBL2105728 | CRENOLANIB | C26H29N5O2 | Crenolanib; ARO 002; ARO- | | FLT3 |
| | | | | 002; CP 868596; CP- | | |
| | | | | 868,596; CP-868596 | | |
| HWXVIOGONBBTBY-ONEGZZNKSA-N | CHEMBL2035187 | PACRITINIB | C28H32N4O3 | Pacritinib; SB-1518; ONX- | | FLT3 |
| | | | | 0803; SB1518 | | |
| PHXJVRSECIGDHY-UHFFFAOYSA-N | CHEMBL1171837 | PONATINIB | C29H27F3N6O | Ponatinib; AP-24534 | Iclusig | FLT3 |
| GBJVVSCPOBPEIT-UHFFFAOYSA-N | CHEMBL415049 | BARASERTIB | C26H31FN7O6P | Barasertib; AZD-1152; AZD | | FLT3 |
| | | | | 1152; AZD1152 | | |
| BLQYVHBZHAISJM-CMDGGOBGSA-N | CHEMBL482968 | ENMD-2076 | C21H25N7 | Enmd 2076; ENMD-2076 | | FLT3 |
| SCMLRESZJCKCTC-KMYQRJGFSA-N | CHEMBL290352 | CEP-1347 | C33H33N3O5S2 | CEP-1347; KT-1575; KT- | | FLT3 |
| | | | | 7515; KT7515 | | |
| NHHQJBCNYHBUSI-UHFFFAOYSA-N | CHEMBL475251 | R-406 | C22H23FN6O5 | R-406 | | FLT3 |
| GCIKSSRWRFVXBI-UHFFFAOYSA-N | CHEMBL572878 | TOZASERTIB | C23H28N8OS | Tozasertib; MK-045; MK- | | FLT3 |
| | | | | 0457; VX-68; VX-680 | | |
| YYLKKYCXAOBSRM-JXMROGBWSA-N | CHEMBL1908397 | KW-2449 | C20H20N4O | Kw 2449; KW-2449 | | FLT3 |
| JOOXLOJCABQBSG-UHFFFAOYSA-N | CHEMBL1287853 | FEDRATINIB | C27H36N6O3S | Fedratinib; SAR- | | FLT3 |
| | | | | 302503; TG101348; TG- | | |
| | | | | 101348 | | |
| XZXHXSATPCNXJR-ZIADKAODSA-N | CHEMBL502835 | NINTEDANIB | C31H33N5O4 | Nintedanib; Intedanib; BIBF- | Ofev; Vargatef | FLT3 |
| | | | | 1120; BIBF1120 | | |
| GYQYAJJFPNQOOW-UHFFFAOYSA-N | CHEMBL3301622 | GILTERITINIB | C29H44N8O3 | Gilteritinib; ASP-2215 | | FLT3 |
| QFCXANHHBCGMAS-UHFFFAOYSA-N | CHEMBL2079588 | TELATINIB | C20H16ClN5O3 | Telatinib; BAY-57-9352; BAY | | FLT4 |
| | | | | 57-9352; BAY-579352 | | |
| WOSKHXYHFSIKNG-UHFFFAOYSA-N | CHEMBL1289601 | LENVATINIB | C21H19ClN4O4 | Lenvatinib; E-7080; ER- | Kisplyx; Lenvima | FLT4 |
| | | | | 203492-00 | | |
| JCKYGMPEJWAADB-UHFFFAOYSA-N | CHEMBL515 | CHLORAMBUCIL | C14H19Cl2NO2 | Chlorambucil; Chlorbutin; | Leukeran | GFER |
| | | | | CB-1348; NCI-3088; NSC- | | |
| | | | | 3088 | | |
| JOWXJLIFIIOYMS-UHFFFAOYSA-N | CHEMBL3622533 | FIMEPINOSTAT | C23H24N8O4S | Fimepinostat; Cudc | | HDAC1 |
| | | | | 907; CUDC-907; CUDC907 | | |
| PAWIYAYFNXQGAP-UHFFFAOYSA-N | CHEMBL2105763 | QUISINOSTAT | C21H26N6O2 | Quisinostat; JNJ | | HDAC1 |
| | | | | 26481585; JNJ-26481585 | | |
| MAUCONCHVWBMHK-UHFFFAOYSA-N | CHEMBL2103863 | ABEXINOSTAT | C21H23N3O5 | Abexinostat; CRA | | HDAC1 |
| | | | | 024781; CRA 24781; CRA- | | |
| | | | | 024781; PCI 24781; PCI- | | |
| | | | | 24781; PZP- | | |
| | | | | 115891; PZP115891; S- | | |
| | | | | 78454 | | |
| YALNUENQHAQXEA-UHFFFAOYSA-N | CHEMBL1213492 | Givinostat | C24H27N3O4 | Givinostat | | HDAC1 |
| QRGHOAATPOLDPF-VQFNDLOPSA-N | CHEMBL1801250 | NANATINOSTAT | C20H19FN6O2 | Nanatinostat; CHR- | | HDAC1 |
| | | | | 3996; VRX-3996 | | |
| PTJGLFIIZFVFJV-UHFFFAOYSA-N | CHEMBL353581 | PYROXAMIDE | C13H19N3O3 | Octanedioic acid | | HDAC1 |
| | | | | hydroxyamide pyridin-3- | | |
| | | | | ylamide; Pyroxamide | | |
| OHRURASPPZQGQM-GCCNXGTGSA-N | CHEMBL343 | ROMIDEPSIN | C24H36N4O6S2 | Romidepsin; Depsipeptide; | Chromadax; Istodax | HDAC1 |
| | | | | FK-228; FR-901228; NSC- 630176 | | |
| HRNLUBSXIHFDHP-UHFFFAOYSA-N | CHEMBL272980 | MOCETINOSTAT | C23H20N6O | Mocetinostat; MGCD- | | HDAC1 |
| | | | | 0103; MGCD0103 | | |
| INVTYAOGFAGBOE-UHFFFAOYSA-N | CHEMBL27759 | ENTINOSTAT | C21H20N4O3 | Entinostat; MS-27-275; MS- | | HDAC1 |
| | | | | 275; MS-275-27; SNDX-275 | | |
| RTKIYFITIVXBLE-QEQCGCAPSA-N | CHEMBL99 | TRICHOSTATIN | C17H22N2O3 | Trichostatin(s); Trichostatin; | | HDAC1 |
| | | | | A-300-I; ANTIBIOTIC A- | | |
| | | | | 300; GNF-Pf-1011 | | |
| NCNRHFGMJRPRSK-MDZDMXLPSA-N | CHEMBL408513 | BELINOSTAT | C15H14N2O4S | Belinostat; NSC726630; PX- | Beleodaq | HDAC1 |
| | | | | 105684; PXD-101 | | |
| MUTBJZVSRNUIHA-UHFFFAOYSA-N | CHEMBL609583 | R-306465 | C19H19N5O4S | R-306465; R306465; JNJ- | | HDAC1 |
| | | | | 16241199 | | |
| YTKUWDBFDASYHO-UHFFFAOYSA-N | CHEMBL487253 | BENDAMUSTINE | C16H21Cl2N3O2 | Bendamustine | | HDAC2 |
| QGZYDVAGYRLSKP-UHFFFAOYSA-N | CHEMBL2364628 | RICOLINOSTAT | C24H27N5O3 | Ricolinostat; ACY- | | HDAC6 |
| | | | | 1215; ACY-63 | | |
| VLIUIBXPEDFJRF-UHFFFAOYSA-N | CHEMBL3693786 | CITARINOSTAT | C24H26ClN5O3 | Citarinostat; ACY- | | HDAC6 |
| | | | | 241; HDAC-IN-2 | | |
| LAMIXXKAWNLXOC-INIZCTEOSA-N | CHEMBL191482 | AR-42 | C18H20N2O3 | Ar-42; (S)-HDAC 42; (S)- | | HDAC6 |
| | | | | HDAC-42; AR42 | | |
| QSLMDECMDJKHMQ-UHFFFAOYSA-N | CHEMBL21731 | MAPROTILINE | C20H23N | Maprotiline; BA-34,276 | | HRH1 |
| | | | | FREE BASE; BA-34276 FREE | | |
| | | | | BASE; BA-34276 [As | | |
| | | | | Hydrochloride] | | |
| YECBIJXISLIIDS-UHFFFAOYSA-N | CHEMBL511 | PYRILAMINE | C17H23N3O | Mepyramine; Pyrilamine | | HRH1 |
| HNJJXZKZRAWDPF-UHFFFAOYSA-N | CHEMBL1411979 | METHAPYRILENE | C14H19N3S | Methapyrilene | | HRH1 |
| MBUVEWMHONZEQD-UHFFFAOYSA-N | CHEMBL639 | AZELASTINE | C22H24ClN3O | Azelastine | | HRH1 |
| RONZAEMNMFQXRA-UHFFFAOYSA-N | CHEMBL654 | MIRTAZAPINE | C17H19N3 | Mirtazapine; ORG-3770 | Mirtazapine; Remeron; | HRH1 |
| | | | | | Remeron Soltab; Zispin | |
| CBEQULMOCCWAQT-WOJGMQOQSA-N | CHEMBL855 | TRIPROLIDINE | C19H22N2 | Triprolidine; Actahist; Actidil; | | HRH1 |
| | | | | Actifed; Allerfed; Corphed; | | |
| | | | | Histafed; Myfed; Myidyl; Trilitron; | | |
| | | | | Triphed | | |
| YANGEESWIGIKOP-UUWRZZSWSA-N | CHEMBL1767164 | GSK-1004723 | C39H49ClN4O2 | Gsk- | | HRH1 |
| | | | | 1004723; Gsk1004723; GSK | | |
| | | | | 1004723 | | |
| PVLJETXTTWAYEW-UHFFFAOYSA-N | CHEMBL94454 | MIZOLASTINE | C24H25FN6O | Mizolastine | Mizollen | HRH1 |
| WUZYKBABMWJHDL-UHFFFAOYSA-N | CHEMBL91397 | RUPATADINE | C26H26ClN3 | Rupatadine; UR-12592 | | HRH1 |
| SEBMTIQKRHYNIT-UHFFFAOYSA-N | CHEMBL946 | AZATADINE | C20H22N2 | Azatadine; Optimine; Trinalin | | HRH1 |
| JJCFRYNCJDLXIK-UHFFFAOYSA-N | CHEMBL516 | CYPROHEPTADINE | C21H21N | Cyproheptadine | | HRH1 |
| ZQDWXGKKHFNSQK-UHFFFAOYSA-N | CHEMBL896 | HYDROXYZINE | C21H27ClN2O2 | Hydroxyzine; Atarax; Orgatrax; | | HRH1 |
| | | | | NSC-169188 | | |
| DERZBLKQOCDDDZ-JLHYYAGUSA-N | CHEMBL43064 | CINNARIZINE | C26H28N2 | Cinnarizine; Stugeron; | Marzine R. F.; Mylan Travel | HRH1 |
| | | | | Stunarone; Stutgin; 516 | Sickness; Stugeron; Stugeron | |
| | | | | MD; 516-MD; R-1575; R-516 | Fte | |
| JAUOIFJMECXRGI-UHFFFAOYSA-N | CHEMBL1172 | DESLORATADINE | C19H19ClN2 | Desloratadine; SCH- | Aerius; Clarinex; Desloratadine; | HRH1 |
| | | | | 34117; Loratadine related | Midetorin; Neoclarityn | |
| | | | | compound a; Loratadine | | |
| | | | | related compound a rs | | |
| UVKZSORBKUEBAZ-UHFFFAOYSA-N | CHEMBL648 | CYCLIZINE | C18H22N2 | Cyclizine; Marezine | | HRH1 |
| MJJALKDDGIKVBE-UHFFFAOYSA-N | CHEMBL305660 | EBASTINE | C32H39NO2 | Ebastine; LAS W-090; LAS- | Kestine | HRH1 |
| | | | | W-090; RP-64305 | | |
| GIJXKZJWITVLHI-PMOLBWCYSA-N | CHEMBL1201203 | BENZTROPINE | C21H25NO | Benzatropine; Benztropine; | | HRH1 |
| | | | | NK-02 | | |
| MVMQESMQSYOVGV-UHFFFAOYSA-N | CHEMBL22108 | DIMETHINDENE | C20H24N2 | Dimetindene | | HRH1 |
| ZKLPARSLTMPFCP-OAQYLSRUSA-N | CHEMBL1201191 | LEVOCETIRIZINE | C21H25ClN2O3 | Levocetirizine; Cetirizine | Xyzal | HRH1 |
| | | | | (r)-form | | |
| ZKLPARSLTMPFCP-UHFFFAOYSA-N | CHEMBL1000 | CETIRIZINE | C21H25ClN2O3 | Cetirizine; AC-170 | | HRH1 |
| CBHCDHNUZWWAPP-UHFFFAOYSA-N | CHEMBL395110 | MEPAZINE | C19H22N2S | Pecazine | | HRH1 |
| GUGOEEXESWIERI-UHFFFAOYSA-N | CHEMBL17157 | TERFENADINE | C32H41NO2 | Terfenadine; RMI-9918 | Antihistamine; Histafen; Histafen | HRH1 |
| | | | | | 120; Seldane; Terfenor; Terfenor | |
| | | | | | Fte; Terfex; Terfinax | |
| | | | | | 120; Terfinax | |
| | | | | | 60; Triludan; Triludan Fte | |
| PWWVAXIEGOYWEE-UHFFFAOYSA-N | CHEMBL643 | PROMETHAZINE | C17H20N2S | Promethazine; Phenergan; | | HRH1 |
| | | | | Promethacon; Promethegan; | | |
| | | | | Remsed; Zipan-25; Zipan- | | |
| | | | | 50; NSC-30321; RP-3277 | | |
| SOYKEARSMXGVTM-HNNXBMFYSA-N | CHEMBL1201353 | DEXCHLORPHENIRAMINE | C16H19ClN2 | Dexchlorpheniramine; | | HRH1 |
| | | | | Chlorpheniramine d-form | | |
| SOYKEARSMXGVTM-UHFFFAOYSA-N | CHEMBL505 | CHLORPHENIRAMINE | C16H19ClN2 | Chlorphenamine; Chlorpheniramine; | | HRH1 |
| | | | | Antagonate; Chlor- | | |
| | | | | Trimeton; Isoclor; Kloromin; | | |
| | | | | Phenetron; Teldrin | | |
| QWGDMFLQWFTERH-UHFFFAOYSA-N | CHEMBL1113 | AMOXAPINE | C17H16ClN3O | Amoxapine; CL-67772 | Amoxapine; Asendin; Asendis; | HRH1 |
| | | | | | Defanyl | |
| ZCVMWBYGMWKGHF-UHFFFAOYSA-N | CHEMBL534 | KETOTIFEN | C19H19NOS | Ketotifen; (R)-Ketotifen | | HRH1 |
| SUPVGFZUWFMATN-UHFFFAOYSA-N | CHEMBL200102 | PU-H71 | C18H21IN6O2S | Pu-h71; PU-H 71; PU-H- | | HSP90AA1 |
| | | | | 71; PU-H71; PUH-71 | | |
| AYUNIORJHRXIBJ-TXHRRWQRSA-N | CHEMBL109480 | TANESPIMYCIN | C31H43N3O8 | Tanespimycin; 17- | | HSP90AA1 |
| | | | | AAG; BMS-722782; CNF- | | |
| | | | | 1010; CNF1010; KOS- | | |
| | | | | 953; NSC-330507 | | |
| NDAZATDQFDPQBD-UHFFFAOYSA-N | CHEMBL252164 | LUMINESPIB | C26H31N3O5 | Luminespib; NVP-AUY- | | HSP90AA1 |
| | | | | 922; NVP-AUY922; NVP- | | |
| | | | | AUY922-NX; VER-52296 | | |
| RVAQIUULWULRNW-UHFFFAOYSA-N | CHEMBL2103879 | GANETESPIB | C2020N4HO3 | Ganetespib; STA 9090; STA- | | HSP90AA1 |
| | | | | 9090 | | |
| QULDDKSCVCJTPV-UHFFFAOYSA-N | CHEMBL467399 | BIIB021 | C14H15ClN6O | Biib021; Cnf2024; BIIB | | HSP90AA1 |
| | | | | 021; BIIB-021; CNF2024 | | |
| KUFRQPKVAWMTJO-LMZWQJSESA-N | CHEMBL383824 | ALVESPIMYCIN | C32H48N4O8 | Alvespimycin; NSC-707545 | | HSP90AA1; |
| | | | | | | HSP90AA1 |
| KUFRQPKVAWMTJO-LMZWQJSESA-N | CHEMBL383824 | ALVESPIMYCIN | C32H48N4O8 | Alvespimycin; NSC-707545 | | HSP90AA1; |
| | | | | | | HSP90AA1 |
| QTQAWLPCGQOSGP-KSRBKZBZSA-N | CHEMBL278315 | GELDANAMYCIN | C29H40N2O9 | Geldanamycin; NSC- | | HSP90AB1 |
| | | | | 122750 | | |
| PHLBKPHSAVXXEF-UHFFFAOYSA-N | CHEMBL621 | TRAZODONE | C19H22ClN5O | Trazodone; Desyrel; | | HTR2A |
| | | | | Trialodine; J10.767K | | |
| AQRLDDAFYYAIJP-UHFFFAOYSA-N | CHEMBL1215661 | PRUVANSERIN | C22H21FN4O | Pruvanserin; LY- | | HTR2A |
| | | | | 2422347; EMD- | | |
| | | | | 281014; EMD-390920; LSN- | | |
| | | | | 2422347; LY-2420586; LY- | | |
| | | | | 2422347 FREE | | |
| | | | | BASE; LY2420586 | | |
| COSPVUFTLGQDQL-UHFFFAOYSA-N | CHEMBL598172 | NELOTANSERIN | C18H15BrF2N4O2 | Nelotanserin; APD-125 | | HTR2A |
| HXTGXYRHXAGCFP-OAQYLSRUSA-N | CHEMBL74355 | VOLINANSERIN | C22H28FNO3 | Volinanserin; MDL- | | HTR2A |
| | | | | 100907; M-100907; MDL- | | |
| | | | | 100.907 | | |
| IKBKZGMPCYNSLU-RGVLZGJSSA-N | CHEMBL76370 | TEGASEROD | C16H23N5O | Tegaserod; HTF-919; SDZ- | Zelmac | HTR2A |
| | | | | HTF-919 | | |
| HXLOHDZQBKCUCR-WOZUAGRISA-N | CHEMBL2087337 | VELUSETRAG | C25H36N4O5S | Velusetrag; TD-5108 | | HTR2A |
| ZEOQUKRCASTCFR-UHFFFAOYSA-N | CHEMBL1084617 | TEMANOGREL | C24H28N4O4 | Temanogrel; APD-791 | | HTR2A |
| QZAYGJVTTNCVMB-UHFFFAOYSA-N | CHEMBL39 | SEROTONIN | C10H12N2O | Serotonin; 5-HT; 5- | | HTR2A |
| | | | | Hydroxytryptamine | | |
| VAYOSLLFUXYJDT-RDTXWAMCSA-N | CHEMBL263881 | LYSERGIDE | C2025N3HO | Lysergide; Lysergic Acid | | HTR2A |
| | | | | Diethylamide; LSD; Lysergic | | |
| | | | | acid diethylamide tartrateci | | |
| KPJZHOPZRAFDTN-NQUBZZJWSA-N | CHEMBL485253 | METHYSERGIDE | C21H27N3O2 | Methysergide | Deseril | HTR2A |
| WVVSZNPYNCNODU-XTQGRXLLSA-N | CHEMBL119443 | ERGONOVINE | C19H23N3O2 | Ergometrine | | HTR2A |
| VRBKIVRKKCLPHA-UHFFFAOYSA-N | CHEMBL623 | NEFAZODONE | C25H32ClN5O2 | Nefazodone | | HTR2A |
| UNBRKDKAWYKMIV-QWQRMKEZSA-N | CHEMBL1201356 | METHYLERGONOVINE | C20H25N3O2 | Methylergometrine; | | HTR2A |
| | | | | Methylergonovine | | |
| LDCYZAJDBXYCGN-VIFPVBQESA-N | CHEMBL350221 | OXITRIPTAN | C11H12N2O3 | Oxitriptan; 5- | Levotonine | HTR2A |
| | | | | hydroxytryptophan I- | | |
| | | | | form; 5-hydroxy-I- | | |
| | | | | tryptophan | | |
| XTTZERNUQAFMOF-QMMMGPOBSA-N | CHEMBL360328 | LORCASERIN | C11H14ClN | Lorcaserin | | HTR2A |
| KORNTPPJEAJQIU-KJXAQDMKSA-N | CHEMBL1201087 | CABERGOLINE | C26H37N5O2 | Cabergoline; FCE-21336 | Cabaser; Cabergoline; Dostinex | HTR2B |
| KQKPFRSPSRPDEB-UHFFFAOYSA-N | CHEMBL128 | SUMATRIPTAN | C14H21N3O2S | Sumatriptan; N02CC01 | Imitrex; Sumatriptan; Tosymra | HTR2C |
| VHFVKMTVMIZMIK-UHFFFAOYSA-N | CHEMBL478 | CHLOROPHENYL- | C10H13ClN2 | 1-M-Chlorophenyl | | HTR2C |
| | | PIPERAZINE | | Piperazine | | |
| | | | | Metabolite; | | |
| | | | | Chlorophenylpiperazine; mCPP | | |
| NPTIPEQJIDTVKR-STQMWFEESA-N | CHEMBL2110670 | VABICASERIN | C15H20N2 | Vabicaserin | | HTR2C |
| YBAWYTYNMZWMMJ-UHFFFAOYSA-N | CHEMBL3286580 | IDALOPIRDINE | C20H19F5N2O | Idalopirdine; LU- | | HTR6 |
| | | | | AE58054; LU AE-58054; LU- | | |
| | | | | AE-58054 | | |
| NXQGEDVQXVTCDA-UHFFFAOYSA-N | CHEMBL2103880 | CERLAPIRDINE | C22H23N3O3S | Cerlapirdine; SAM-531; PF- | | HTR6 |
| | | | | 05212365; SAM-531 FREE | | |
| | | | | BASE; WAY-262,531; WAY- | | |
| | | | | 262531 | | |
| JJZFWROHYSMCMU-UHFFFAOYSA-N | CHEMBL1083390 | INTEPIRDINE | C19H19N3O2S | Intepirdine; SB- | | HTR6 |
| | | | | 742457; GSK- | | |
| | | | | 742457; GSK742457; RVT- | | |
| | | | | 101 | | |
| MTTHRRVVGMPYQG-ZDUSSCGKSA-N | CHEMBL1082508 | LANDIPIRDINE | C18H19FN2O3S | Landipirdine; R0- | | HTR6 |
| | | | | 5025181; R0-5025181- | | |
| | | | | 000; R05025181; R05025181- | | |
| | | | | 000; RO5025181; RO50251 | | |
| | | | | 81-000; SYN-120; SYN120 | | |
| JNODQFNWMXFMEV-UHFFFAOYSA-N | CHEMBL589390 | LATREPIRDINE | C21H25N3 | Latrepirdine; Dimebolin | | HTR7 |
| UKJPMZGILXATGT-UHFFFAOYSA-N | CHEMBL4205783 | JNJ-18038683 | C20H20ClN3 | | | HTR7 |
| LQVXSNNAFNGRAH-QHCPKHFHSA-N | CHEMBL575448 | BMS-754807 | C23H24FN9O | BMS-754807 | | IGF1R |
| VERWOWGGCGHDQE-UHFFFAOYSA-N | CHEMBL2403108 | CERITINIB | C28H36ClN5O3S | Ceritinib; LDK- | Zykadia | IGF1R |
| | | | | 378; LDK378; NVP-LDK-378- | | |
| | | | | NX; NVP-LDK378-NX | | |
| ALKJNCZNEOTEMP-UHFFFAOYSA-N | CHEMBL3545085 | XL-228 | C22H31N9O | XL-228; XL228 | | IGF1R |
| PKCDDUHJAFVJJB-VLZXCDOPSA-N | CHEMBL1091644 | LINSITINIB | C26H23N5O | Linsitinib; ASP-7487; OSI- | | IGF1R |
| | | | | 906; OSI-906AA | | |
| GZGLPBNOIFLLRE-UHFFFAOYSA-N | CHEMBL129857 | AS-602868 | C18H14N4O3S | AS-602868; SPC-839 | | IKBKB |
| IXTMWRCNAAVVAI-UHFFFAOYSA-N | CHEMBL473 | DOFETILIDE | C19H27N3O5S2 | Dofetilide; UK-68798 | Dofetilide; Tikosyn | KCNH2 |
| ALOBUEHUHMBRLE-UHFFFAOYSA-N | CHEMBL533 | IBUTILIDE | C20H36N2O3S | Ibutilide | | KCNH2 |
| FOHHNHSLJDZUGQ-UHFFFAOYSA-N | CHEMBL1107 | HALOFANTRINE | C26H30Cl2F3NO | Halofantrine | | KCNH2 |
| SGTNSNPWRIOYBX-UHFFFAOYSA-N | CHEMBL6966 | VERAPAMIL | C27H38N2O4 | Verapamil; Calan; Covera- | | KCNH2 |
| | | | | HS; Iproveratril; Isoptin; | | |
| | | | | Tarka; Verelan; CP-16533-1; CP- | | |
| | | | | 165331; D-365; NSC- | | |
| | | | | 272306NA | | |
| LDXYBEHACFJIEL-HNNXBMFYSA-N | CHEMBL1929396 | ANAGLIPTIN | C19H25N7O2 | Anagliptin; Sk-0403; CWP- | | KCNH2 |
| | | | | 403; SK-0403 | | |
| WSEQXVZVJXJVFP-UHFFFAOYSA-N | CHEMBL549 | CITALOPRAM | C20H21FN2O | Citalopram; Celexa | | KCNH2 |
| LOUPRKONTZGTKE-LHHVKLHASA-N | CHEMBL1294 | QUINIDINE | C20H24N2O2 | Quinidine | | KCNH2 |
| WSEQXVZVJXJVFP-FQEVSTJZSA-N | CHEMBL1508 | ESCITALOPRAM | 21FNC20H2O | Escitalopram; LU-26-054 | Cipralex | KCNH2 |
| LOUPRKONTZGTKE-WZBLMQSHSA-N | CHEMBL170 | QUININE | C20H24N2O2 | Quinine; NSC- | | KCNH2 |
| | | | | 192949; WR297608 | | |
| HXHAJRMTJXHJJZ-UHFFFAOYSA-N | CHEMBL253969 | OSI-632 | C20H24BrF2N5O3S | 547632; OSI-632; CP- | | KDR |
| | | | | Cp-547,632; CP- | | |
| | | | | 547,632; CP-632; PAN | | |
| | | | | 90806; PAN- | | |
| | | | | 90806; PAN90806 | | |
| FGTCROZDHDSNIO-UHFFFAOYSA-N | CHEMBL1614710 | OSI-930 | C22H16F3N3O2S | Osi 930; OSI-930 | | KDR |
| WPEWQEMJFLWMLV-UHFFFAOYSA-N | CHEMBL3186534 | RIVOCERANIB | C24H23N5O | Rivoceranib; Apatinib; YN96 | | KDR |
| | | | | 8D1 | | |
| UOVCGJXDGOGOCZ-UHFFFAOYSA-N | CHEMBL1980391 | RG1530 | C18H14ClFN4O | R-1530; RG-1530 | | KDR |
| JXSVVZKPEDIRTN-DHZHZOJOSA-N | CHEMBL3545335 | AG-13958 | C26H22FN7O | AG-13958 | | KDR |
| WLAVZAAODLTUSW-UHFFFAOYSA-N | CHEMBL3989926 | SITRAVATINIB | C33H29F2N5O4S | Sitravatinib; MG-516; MG- | | KDR |
| | | | | 91516; MGCD- | | |
| | | | | 516; MGCD516 | | |
| GNNDEPIMDAZHRQ-UHFFFAOYSA-N | CHEMBL3545365 | ALTIRATINIB | C26H21F3N4O4 | Altiratinib; DCC-2701; DP- | | KDR |
| | | | | 5164 | | |
| FNHKPVJBJVTLMP-UHFFFAOYSA-N | CHEMBL1946170 | REGORAFENIB | C21H15ClF4N4O3 | Regorafenib; BAY-73- | Stivarga | KIT |
| | | | | 4506; BAY-734506; BAY- | | |
| | | | | 734506 monohydrate | | |
| KTEIFNKAUNYNJU-GFCCVEGCSA-N | CHEMBL601719 | CRIZOTINIB | C21H22Cl2FN5O | Crizotinib; PF- | Xalkori | LCK |
| | | | | 02341066; PF-2341066 | | |
| QFJCIRLUMZQUOT-HPLJOQBZSA-N | CHEMBL413 | SIROLIMUS | C51H79NO13 | Sirolimus; Rapamycin; AY- | Rapamune; Sirolimus | LCK |
| | | | | 22989; L04AA10; NSC- | | |
| | | | | 226080; SM-88 | | |
| | | | | COMPONENT | | |
| | | | | SIROLIMUS; WY-090217 | | |
| OUKYUETWWIPKQR-UHFFFAOYSA-N | CHEMBL217092 | SARACATINIB | C27H32ClN5O5 | Saracatinib; AZD-0530; AZ- | | LCK |
| | | | | 10353926 | | |
| ZGBAJMQHJDFTQJ-DEOSSOPVSA-N | CHEMBL206834 | BAFETINIB | C30H31F3N8O | Bafetinib; CNS-9; INNO- | | LYN |
| | | | | 406; NS-187 | | |
| ZMELOYOKMZBMRB-DLBZAZTESA-N | CHEMBL514201 | TALMAPIMOD | C27H30ClFN4O3 | Talmapimod; SCIO-469 | | MAPK12 |
| VEPKQEUBKLEPRA-UHFFFAOYSA-N | CHEMBL119385 | NEFLAMAPIMOD | C19H9Cl2F2N3OS | Neflamapimod; VX- | | MAPK12 |
| | | | | 745; 745; VD-31; VD- | | |
| | | | | 31,745; VD-31745; VRT- | | |
| | | | | 031745 | | |
| REFJWTPEDVJJIY-UHFFFAOYSA-N | CHEMBL50 | QUERCETIN | C15H10O7 | 9219; NSC-57655; NSC- | | MAPK12 |
| | | | | Quercetin; C.I. Natural Red | | |
| | | | | 1; Meletin; Sophoretin; Xant | | |
| | | | | haurine; LDN | | |
| | | | | 0052529; LDN- | | |
| | | | | 0052529; NSC 57655; NSC | | |
| | | | | 9219 | | |
| GFMMXOIFOQCCGU-UHFFFAOYSA-N | CHEMBL105442 | CI-1040 | C17H14ClF2IN2O2 | Ci 1040; CI-1040; PD- | | MAPK12 |
| | | | | 18435; PD-184352 | | |
| MVCOAUNKQVWQHZ-UHFFFAOYSA-N | CHEMBL103667 | DORAMAPIMOD | C31H37N5O3 | Doramapimod; BIRB- | | MAPK12 |
| | | | | 796; BIRB 796 BS; BIRB-796 | | |
| | | | | BS | | |
| IFGWYHGYNVGVRB-UHFFFAOYSA-N | CHEMBL1088750 | ARRY-797 | C22H26F2N4O2 | ARRY-797; ARRY-371797 | | MAPK14 |
| KCAJXIDMCNPGHZ-UHFFFAOYSA-N | CHEMBL1088751 | PH-797804 | C22H19BrF2N2O3 | PH-797804; PHA-797804 | | MAPK14 |
| FYSRKRZDBHOFAY-UHFFFAOYSA-N | CHEMBL1090090 | VX-702 | C19H12F4N4O2 | VX-702 | | MAPK14 |
| KKYABQBFGDZVNQ-UHFFFAOYSA-N | CHEMBL1088752 | LOSMAPIMOD | C22H26FN3O2 | Losmapimod; GW856553; | | MAPK14 |
| | | | | GSKAHAB; GW- | | |
| | | | | 856553X; GW856553X | | |
| HEKAIDKUDLCBRU-UHFFFAOYSA-N | CHEMBL363648 | TAK-715 | C24H21N3OS | TAK-715; TAK 715 | | MAPK14 |
| JYYLVUFNAHSSFE-UHFFFAOYSA-N | CHEMBL1090089 | PAMAPIMOD | C19H20F2N4O4 | Pamapimod; R-1503; JTT- | | MAPK14 |
| | | | | 705; Ro-4402257 | | |
| KKKRKRMVJRHDMG-UHFFFAOYSA-N | CHEMBL1230122 | R-1487 | C19H18F2N4O3 | | | MAPK14 |
| RTDCVLCTBQDLBW-UHFFFAOYSA-N | CHEMBL3695568 | JNJ-49095397 | C34H36N6O4 | Jnj-49095397; Rv568; JNJ- | | MAPK14 |
| | | | | 49095397; RV-568; RV568 | | |
| ROSNVSQTEGHUKU-UHFFFAOYSA-N | CHEMBL440498 | CTS-1027 | C19H20ClNO6S | Cts-1027; RO-1130830; RS- | | MMP13 |
| | | | | 130830 | | |
| CGTADGCBEXYWNE-JUKNQOCSSA-N | CHEMBL219410 | ZOTAROLIMUS | C52H79N5O12 | Zotarolimus; ABT-578 | | MTOR |
| BMMXYEBLEBULND-UHFFFAOYSA-N | CHEMBL3218578 | BGT-226 | C28H25F3N6O2 | BGT-226; NVP-BGT226 | | MTOR |
| RGJOJUGRHPQXGF-INIZCTEOSA-N | CHEMBL2331680 | RG-7603 | C24H32N6O3 | RG-7603; GDC-0349 | | MTOR |
| QJJXYPPXXYFBGM-LFZNUXCKSA-N | CHEMBL269732 | TACROLIMUS | C44H69NO12 | Tacrolimus | Prograf; Protopic; Tacrolimus | MTOR |
| | | | | monohydrate; FK- | | |
| | | | | 506; FK506; FR-900506 | | |
| HKVAMNSJSFKALM-GKUWKFKPSA-N | CHEMBL1908360 | EVEROLIMUS | C53H83NO14 | Everolimus; RAD 666; RAD- | Afinitor; Afinitor | MTOR |
| | | | | 001; RAD- 666; | Disperz; Certican; Votubia; | |
| | | | | RAD001; SDZ-RAD | Zortress | |
| WXUUCRLKXQMWRY-UHFFFAOYSA-N | CHEMBL1258517 | PKI-179 | C25H28N8O3 | PKI-179 | | MTOR |
| KVLFRAWTRWDEDF-IRXDYDNUSA-N | CHEMBL1801204 | AZD-8055 | C25H31N5O4 | AZD-8055; AZ- | | MTOR |
| | | | | 12600000; | | |
| | | | | AZD8055; AZD8055 | | |
| JOGKUKXHTYWRGZ-UHFFFAOYSA -N | CHEMBL1879463 | DACTOLSIB | C30H23N5O | Dactolisib; BEZ- | | MTOR |
| | | | | 235; BEZ235; NVP- | | |
| | | | | BEZ235; NVP-BEZ235-NX | | |
| DWZAEMINVBZMHQ-UHFFFAOYSA-N | CHEMBL592445 | GEDATOLISIB | C32H41N9O4 | Gedatolisib; PF- | | MTOR |
| | | | | 05212384; PKI-587 | | |
| CGBJSGAELGCMKE-UHFFFAOYSA-N | CHEMBL1236962 | OMIPALISIB | C25H17F2N5O3S | Omipalisib; GSK2126458 | | MTOR |
| JUSFANSTBFGBAF-IRXDYDNUSA-N | CHEMBL2336325 | VISTUSERTIB | C25H30N6O3 | Vistusertib; Azd-2014; AZD- | | MTOR |
| | | | | 2014; AZD2014 | | |
| XDLYKKIQACFMJG-WKILWMFISA-N | CHEMBL1234354 | PF-04691502 | C22H27N5O4 | PF-04691502; PF-4691502 | | MTOR |
| YOVVNOKCSKSHKT-HNNXBMFYSA-N | CHEMBL1922094 | APITOLISIB | C23H30N8O3S | Apitolisib; RG-7422; G- | | MTOR |
| | | | | 038390; G-038390.1; GDC- | | |
| | | | | 0980; GDC-0980.1 | | |
| GYLDXIAOMVERTK-UHFFFAOYSA-N | CHEMBL354509 | SAPANISERTIB | C15H15N7O | Ink-128; MLN- | | MTOR |
| | | | | 0128; Sapanisertib; INK- | | |
| | | | | 128; INK128; MLN0128; TAK-228 | | |
| GXJABQQUPOEUTA-RDJZCZTQSA-N | CHEMBL325041 | BORTEZOMIB | C19H25BN4O4 | Bortezomib; Bortezomib | Velcade | NFKB1 |
| | | | | hydrate; Velcade; LDP- | | |
| | | | | 341; NSC-681239; PS-341 | | |
| MXAYKZJJDUDWDS-LBPRGKRZSA-N | CHEMBL2141296 | IXAZOMIB | C14H19BCl2N2O4 | Ixazomib; MLN-2238; MLN- | Ninlaro | NFKB1 |
| | | | | 9708 FREE BASE; MLN2238 | | |
| IFPPYSWJNWHOLQ-UHFFFAOYSA-N | CHEMBL49120 | PD-0166285 | C26H27Cl2N5O2 | TCMDC-140940; PD- | | PDGFRA |
| | | | | 0166285 | | |
| YABJJWZLRMPFSI-UHFFFAOYSA-N | CHEMBL558752 | RAF-265 | C24H16F6N6O | Raf 265; RAF-265; CHIR- | | PDGFRB |
| | | | | 265; NVP-RAF265; RAF265 | | |
| UJIAQDJKSXQLIT-UHFFFAOYSA-N | CHEMBL230011 | TG100-115 | C18H14N6O2 | TG100-115 | | PIK3C2B |
| BEUQXVWXFDOSAQ-UHFFFAOYSA-N | CHEMBL2380708 | TASELISIB | C24H28N8O2 | Taselisib; GDC-0032; RG- | | PIK3CA |
| | | | | 7604 | | |
| LHNIIDJUOCFXAP-UHFFFAOYSA-N | CHEMBL521851 | PICTILISIB | C23H27N7O3S2 | Pictilisib; RG-7321; CDC- | | PIK3CA |
| | | | | 0941; GDC 0941; GDC-0941 | | |
| CWHUFRVAEUJCEF-UHFFFAOYSA-N | CHEMBL2017974 | BUPARLISIB | C18H21F3N6O2 | Buparlisib; BKM-120; BKM- | | PIK3CA |
| | | | | 120NX; BKM120-NX; NVP- | | |
| | | | | BKM-120; NVP-BKM120 | | |
| PZBCKZWLPGJMAO-UHFFFAOYSA-N | CHEMBL3218576 | COPANLISIB | C23H28N8O4 | Copanlisib; BAY-80- | | PIK3CA |
| | | | | 6946; BAY 80-6946; BAY80- | | |
| | | | | 6946 | | |
| STUWGJZDJHPWGZ-LBPRGKRZSA-N | CHEMBL2396661 | ALPELISIB | C19H22F3N5O2S | Alpelisib; BYL-719; NVP- | Piqray | PIK3CA |
| | | | | BYL719 | | |
| ADGGYDAFIHSYFI-UHFFFAOYSA-N | CHEMBL4084907 | BIMIRALISIB | C17H20F3N7O2 | Bimiralisib; Pqr309; NC- | | PIK3CA |
| | | | | B5; NCB5; PI3K-IN-2; PQR- | | |
| | | | | 309; PQR309 | | |
| ZGRDYKFVDCFJCZ-UHFFFAOYSA-N | CHEMBL3894518 | AZD-8835 | C22H31N9O3 | Azd 8835; Azd- | | PIK3CA |
| | | | | 8835; Azd8835; AZD- | | |
| | | | | 8835; AZD8835 | | |
| IRTDIKMSKMREGO-OAHLLOKOSA-N | CHEMBL2165191 | AZD-6482 | C22H24N4O4 | Azd 6482; AZD- | | PIK3CB |
| | | | | 6482; AZD6482 | | |
| XTKLTGBKIDQGQL-UHFFFAOYSA-N | CHEMBL3188551 | GSK-2636771 | C22H22F3N3O3 | Gsk | | PIK3CB |
| | | | | 2636771; GSK2636771; GSK- | | |
| | | | | 2636771 | | |
| MCIDWGZGWVSZMK-UHFFFAOYSA-N | CHEMBL2216859 | NEMIRALISIB | C26H28N6O | 2269557; GSK-2269557 | | PIK3CD |
| | | | | Nemiralisib; GSK- | | |
| | | | | FREE BASE; GSK- | | |
| | | | | 2269557A; GSK2269557A | | |
| XDSXYMOZKDUASY-INIZCTEOSA-N | CHEMBL3928407 | GS-9901 | C22H17ClFN9O | Gs 9901; Gs-9901 | | PIK3CD |
| MWKYMZXCGYXLPL-ZDUSSCGKSA-N | CHEMBL3643413 | LENIOLISIB | C21H25F3N6O2 | CDZ173-NX; Leniolisib | | PIK3CD |
| IFSDAJWBUCMOAH-HNNXBMFYSA-N | CHEMBL2216870 | IDELALISIB | C22H18FN7O | Idelalisib; CAL-101; GS- | Zydelig | PIK3CD |
| | | | | 11CAL-101; GS-1101 | | |
| SJVQHLPISAIATJ-ZDUSSCGKSA-N | CHEMBL3039502 | DUVELISIB | C22H17ClN6O | Duvelisib; INK-1147; INK- | Copiktra | PIK3CD |
| | | | | 1197; IPI-145 | | |
| XUMALORDVCFWKV-IBGZPJMESA-N | CHEMBL3984425 | IPI-549 | C30H24N8O2 | Ipi-549; IPI-549 | | PIK3CG |
| GWRSATNRNFYMDI-UHFFFAOYSA-N | CHEMBL2392545 | TAK-960 | C27H34F3N7O3 | TAK-960; TAK960 | | PLK1 |
| QHLVBNKYJGBCQJ-UHFFFAOYSA-N | CHEMBL1738758 | ONVANSERTIB | C24H27F3N8O3 | NMS- | | PLK1 |
| | | | | 1286937; Onvansertib; | | |
| | | | | NMS-P937; PCM-075 | | |
| OWBFCJROIKNMGD-BQYQJAHWSA-N | CHEMBL1241855 | RIGOSERTIB | C21H25NO8S | Rigosertib; ON-01910 | | PLK1 |
| SXNJFOWDRLKDSF-STROYTFGSA-N | CHEMBL1233528 | VOLASERTIB | C34H50N8O3 | Volasertib; BI-6727 | | PLK1 |
| ZHJGWYRLJUCMRT-QGZVFWFLSA-N | CHEMBL1908394 | GSK-461364 | C27H28F3N5O2S | Gsk | | PLK1 |
| | | | | 461364; GSK461364; GSK- | | |
| | | | | 461364A | | |
| XQVVPGYIWAGRNI-JOCHJYFZSA-N | CHEMBL513909 | BI-2536 | C28H39N7O3 | Bi 2536; BI-2536 | | PLK1 |
| BKWJAKQVGHWELA-UHFFFAOYSA-N | CHEMBL1976040 | ADAVOSERTIB | C27H32N8O2 | Adavosertib; AZD- | | PLK1 |
| | | | | 1775; AZD | | |
| | | | | 1775; AZD1775; MK-1775 | | |
| OYHQOLUKZRVURQ-HZJYTTRNSA-N | CHEMBL267476 | LINOLEIC ACID | C18H32O2 | Linoleic acid; FEMA NO. | | PPARA |
| | | | | 3380, LINOLEIC ACID-; NSC- | | |
| | | | | 281243; Octadeca-9,12- | | |
| | | | | Dienoic Acid | | |
| ZHKNLJLMDFQVHJ-RUZDIDTESA-N | CHEMBL247951 | PEMAFIBRATE | C28H30N2O6 | Pemafibrate; (R)-K-13675 | | PPARA |
| IPSYPUKKXMNCNQ-PFHKOEEOSA-N | CHEMBL431733 | NAMODENOSON | C18H18ClIN6O4 | CF-102; Chloro-IB- | | PPARD |
| | | | | MECA; CI-IB- | | |
| | | | | MECA; Namodenoson; CL- | | |
| | | | | IB-MECA | | |
| YDBLKRPLXZNVNB-UHFFFAOYSA-N | CHEMBL38943 | GW501516 | C21H18F3NO3S2 | Gw501516; GSK-516; GW- | | PPARD |
| | | | | 1516; GW501516 | | |
| NMRWDFUZLLQSBN-UHFFFAOYSA-N | CHEMBL1236924 | INT131 | C21H12Cl4N2O3S | INT-131; AMG-131; T- | | PPARG |
| | | | | 0903131; T131 | | |
| JCYNMRJCUYVDBC-UHFFFAOYSA-N | CHEMBL3545280 | EFATUTAZONE | C27H26N4O4S | Efatutazone | | PPARG |
| STWITCBWQHTJFJ-XMMPIXPASA-N | CHEMBL490029 | MK-0533 | C28H24F3NO6 | MK-0533; MK0533 | | PPARG |
| YASAKCUCGLMORW-UHFFFAOYSA-N | CHEMBL121 | ROSIGLITAZONE | C18H19N3O3S | Rosiglitazone; Avandamet; | Avandia | PPARG |
| | | | | Avandaryl; Avandia; BRL- | | |
| | | | | 49653; TDZ-01 | | |
| IRLWJILLXJGJTD-UHFFFAOYSA-N | CHEMBL186179 | MURAGLITAZAR | C29H28N2O7 | Muraglitazar; BMS- | | PPARG |
| | | | | 298585; BMS-298585- | | |
| | | | | 01; MK-0478; MK0478 | | |
| ZZCHHVUQYRMYLW-HKBQPEDESA-N | CHEMBL107367 | FARGLITAZAR | C34H30N2O5 | Farglitazar; GI-262570; G1- | | PPARG |
| | | | | 262570; GI-262570X | | |
| PKXWXXPNHIWQHW-RCBQFDQVSA-N | CHEMBL520733 | SEMAGACESTAT | C19H27N3O4 | Semagacestat; LY-450139 | | PSEN2 |
| PSXOKXJMVRSARX-SCSAIBSYSA-N | CHEMBL463981 | BEGACESTAT | C9H8ClF6NO3S2 | Begacestat; GSI-953 | | PSEN2 |
| XEAOPVUAMONVLA-QGZVFWFLSA-N | CHEMBL1090771 | AVAGACESTAT | C20H17ClF4N4O4S | Avagacestat; BMS | | PSEN2 |
| | | | | 708163; BMS-708163; BMS- | | |
| | | | | 708163-01; BMS-70816301 | | |
| VFCRKLWBYMDAED-REWPJTCUSA-N | CHEMBL1770916 | NIROGACESTAT | C27H41F2N5O | Nirogacestat; PF | | PSEN2 |
| | | | | 03084014; PF 3084014; PF- | | |
| | | | | 03084014; PF-3084014 | | |
| VCIBGDSRPUOBOG-QFIPXVFZSA-N | CHEMBL2364614 | LIAFENSINE | C24H22N4 | Liafensine; BMS-820836 | | SLC6A2 |
| XXPANQJNYNUNES-UHFFFAOYSA-N | CHEMBL273575 | NOMIFENSINE | C16H18N2 | Nomifensine | | SLC6A2 |
| ICXJGCSEMJXNQF-ZIAGYGMSSA-N | CHEMBL1173532 | 1-(3,4- | C14H17Cl2NO | | | SLC6A2 |
| | | DICHLOROPHENYL)-6- | | | | |
| | | (METHOXYMETHYL)- | | | | |
| | | 3-AZA- | | | | |
| | | BICYCLO[4.1.0]HEP- | | | | |
| | | TANE (ENANTIOMERIC | | | | |
| | | MIX) | | | | |
| CBQGYUDMJHNJBX-OALUTQOASA-N | CHEMBL180101 | ESREBOXETINE | C19H23NO3 | Esreboxetine; (S,S)- | | SLC6A2 |
| | | | | Reboxetine; Reboxetine | | |
| CBQGYUDMJHNJBX-UHFFFAOYSA-N | CHEMBL14370 | REBOXETINE | C19H23NO3 | Reboxetine | Edronax | SLC6A2 |
| ZEUITGRIYCTCEM-KRWDZBQOSA-N | CHEMBL1175 | DULOXETINE | C18H19NOS | Duloxetine; Cymbalta; LY- | | SLC6A2 |
| | | | | 248686; LY248686 | | |
| VHGCDTVCOLNTBX-QGZVFWFLSA-N | CHEMBL641 | ATOMOXETINE | C17H21NO | Atomoxetine; Strattera | | SLC6A2 |
| ZPXSCAKFGYXMGA-UHFFFAOYSA-N | CHEMBL781 | MAZINDOL | C16H13ClN2O | Mazindol; 42-548; AN- | Mazanor; Sanorex; Teronac | SLC6A2 |
| | | | | 448; SAH-42548; Mazindol civ | | |
| KWTSXDURSIMDCE-UHFFFAOYSA-N | CHEMBL405 | AMPHETAMINE | C9H13N | Amfetamine; Amphetamin | Adzenys ER; Adzenys xr- | SLC6A2 |
| | | | | e; Amphetamine resin | odt; Dyanavel XR | |
| | | | | complex; NSC-27159 | | |
| KWTSXDURSIMDCE-QMMMGPOBSA-N | CHEMBL612 | DEXTROAMPHETAMINE | C9H13N | Dexamfetamine; Dexamphetamine; | Dexedrine; Dextrostat | SLC6A2 |
| | | | | Dextroamphetamine; | | |
| | | | | Dextroamphetamine | | |
| | | | | resin complex; d- | | |
| | | | | Amphetamine; Dexampex; | | |
| | | | | Dexedrine; Dextrostat; | | |
| | | | | Ferndex; NSC-73713 | | |
| GJJFMKBJSRMPLA-UHFFFAOYSA-N | CHEMBL259209 | MILNACIPRAN | C15H22N2O | Milnacipran; Midalcipran | | SLC6A2 |
| GJJFMKBJSRMPLA-DZGCQCFKSA-N | CHEMBL99946 | LEVOMILNACIPRAN | C15H22N2O | Levomilnacipran; F 2695; F- | Fetzima | SLC6A2 |
| | | | | 2695 | | |
| BWPIARFWQZKAIA-UHFFFAOYSA-N | CHEMBL668 | PROTRIPTYLINE | C19H21N | Protriptyline | | SLC6A2 |
| HXWLAJVUJSVENX-HZMVEIRTSA-N | CHEMBL1945778 | IOFLUPANE | C18H23FINO2 | | | SLC6A3 |
| CJOFXWAVKWHTFT-XSFVSMFZSA-N | CHEMBL814 | FLUVOXAMINE | C15H21F3N2O2 | Fluvoxamine; N06AB08 | | SLC6A3 |
| SWUVZKWCOBGPTH-UHFFFAOYSA-N | CHEMBL201960 | PYROVALERONE | C16H23NO | Pyrovalerone | | SLC6A3 |
| NAUWTFJOPJWYOT-UHFFFAOYSA-N | CHEMBL281594 | VANOXERINE | C28H32F2N2O | Vanoxerine; GBR | | SLC6A3 |
| | | | | 12909; GBR-12909 | | |
| AHOUBRCZNHFOSL-YOEHRIQHSA-N | CHEMBL490 | PAROXETINE | C19H20FNO3 | Paroxetine; Paxil; Pexeva; | Paxil [as hydrochloride] | SLC6A3 |
| | | | | BRL-29060 | | |
| MJJDYOLPMGIWND-UHFFFAOYSA-N | CHEMBL471036 | LITOXETINE | C16H19NO | Litoxetine | | SLC6A4 |
| BSMNRYCSBFHEMQ-KCJUWKMLSA-N | CHEMBL592374 | AMITIFADINE | C11H11Cl2N | Amitifadine; DOV- | | SLC6A4 |
| | | | | 21947; EB-1010 | | |
| VCNSPGHSQPMCFF-UHFFFAOYSA-N | CHEMBL505147 | UK-390957 | C16H20N2O3S2 | Uk-390,957; Uk- | | SLC6A4 |
| | | | | 390957; UK-390,957 | | |
| SADQVAVFGNTEOD-UHFFFAOYSA-N | CHEMBL276520 | INDALPI NE | C15H20N2 | Indalpine; LM-5008 | | SLC6A4 |
| YQNWZWMKLDQSAC-UHFFFAOYSA-N | CHEMBL2104993 | VORTIOXETINE | C18H22N2S | Vortioxetine; LU | Brintellix | SLC6A4 |
| | | | | AA21004; LU- | | |
| | | | | AA21004; LUAA21004 | | |
| SGEGOXDYSFKCPT-UHFFFAOYSA-N | CHEMBL439849 | VILAZODONE | C26H27N5O2 | Vilazodone; EMD- | | SLC6A4 |
| | | | | 515259; EMD-68-843 | | |
| SHXWCVYOXRDMCX-UHFFFAOYSA-N | CHEMBL43048 | MIDOMAFETAMINE | C11H15NO2 | Mdma; 3,4- | | SLC6A4 |
| | | | | Methylenedioxymethamphetamine; | | |
| | | | | Methylenedioxy | | |
| | | | | Methamphetamine; | | |
| | | | | Methylenedioxymethamphetamine; | | |
| | | | | Midomafetamine | | |
| INDBQLZJXZLFIT-UHFFFAOYSA-N | CHEMBL506 | PRIMAQUINE | C15H21N3O | Primaquine; NSC- | | SLC6A4 |
| | | | | 27296; SN-13272; WR-2975 | | |
| ZPUCINDJVBIVPJ-LJISPDSOSA-N | CHEMBL370805 | COCAINE | C17H21NO4 | Cocaine; IDS-NC-004; RX- | | SLC6A4 |
| | | | | 0041; RX0041 | | |
| MKXZASYAUGDDCJ-NJAFHUGGSA-N | CHEMBL52440 | DEXTROMETHORPHAN | C18H25NO | Dextromethorphan; Dimetane- | | SLC6A4 |
| | | | | DX | | |
| VGKDLMBJGBXTGI-SJCJKPOMSA-N | CHEMBL809 | SERTRALINE | C17H17Cl2N | Sertraline | | SLC6A4 |
| PNVNVHUZROJLTJ-UHFFFAOYSA-N | CHEMBL637 | VENLAFAXINE | C17H27NO2 | Venlafaxine; Effexor | Alventa XL; Amphero | SLC6A4 |
| | | | | | XL; Bonilux XL; Depefex | |
| | | | | | XL; Efexor; Efexor XL; Foraven | |
| | | | | | XL; Mentaven XL; Politid | |
| | | | | | XL; Ranfaxine XL; Rodomel | |
| | | | | | XL; Sunveniz XL; Tardcaps | |
| | | | | | XL; Tifaxin XL; Tonpular | |
| | | | | | XL; Trixat XL; Vaxalin | |
| | | | | | XL; Venaxx XL; Venlablue | |
| | | | | | XL; Venladex XL; Venlalic | |
| | | | | | XL; Venlaneo XL; Vensir | |
| | | | | | XL; Vexarin XL; Viepax; ViePax | |
| | | | | | XL; Winfex XL | |
| SHZKQBHERIJWAO-AATRIKPKSA-N | CHEMBL11662 | OZAGREL | C13H12N2O2 | Ozagrel; Ozagrel | | TBXAS1 |
| | | | | Sodium; KCT-0809 | | |
| XUTLOCQNGLJNSA-RGVLZGJSSA-N | CHEMBL281398 | TERBOGREL | C23H27N5O2 | Terbogrel; BIBV-308- | | TBXAS1 |
| | | | | SE; BIBV-308SE | | |
| OFDNQWIFNXBECV-VFSYNPLYSA-N | CHEMBL39541 | DOLASTATIN-10 | C42H68N6O6S | Dolastatin 10; Dolastatin- | | TUBB6 |
| | | | | 10; Dolostatin 10 | | |
| QXRSDHAAWVKZLJ-PVYNADRNSA-N | CHEMBL94657 | PATUPILONE | C27H41NO6S | Patupilone; Epothilone | | TUBB6 |
| | | | | b; EPO 906; EPO 906A; EPO- | | |
| | | | | 906; EPO-906A; GNF-PF-193 | | |
| ADDITIONAL FIELDS TO TABLE 1AB | | | | | | |
| SMALL MOLECULE | INHIBITOR OF | ABCB1 | | | | |
| SMALL MOLECULE | INHIBITOR OF | ABCB11 | | | | |
| SMALL MOLECULE | INHIBITOR OF | ABCG2 | | | | |
| SMALL MOLECULE | INHIBITOR OF | ADAM17 | | | | |
| SMALL MOLECULE | INHIBITOR OF | ADRA1A | | | | |
| SMALL MOLECULE | INHIBITOR OF | ADRA1B | | | | |
| SMALL MOLECULE | INHIBITOR OF | ADRA1D | | | | |
| SMALL MOLECULE | INHIBITOR OF | ADRA2A | | | | |
| SMALL MOLECULE | INHIBITOR OF | ADRA2B | | | | |
| SMALL MOLECULE | INHIBITOR OF | ADRA2C | | | | |
| SMALL MOLECULE | INHIBITOR OF | ATXN2 | | | | |
| SMALL MOLECULE | INHIBITOR OF | BACE1 | | | | |
| SMALL MOLECULE | INHIBITOR OF | BACE2 | | | | |
| SMALL MOLECULE | INHIBITOR OF | BLM | | | | |
| SMALL MOLECULE | INHIBITOR OF | BRD2 | | | | |
| SMALL MOLECULE | INHIBITOR OF | BRD3 | | | | |
| SMALL MOLECULE | INHIBITOR OF | BRD4 | | | | |
| SMALL MOLECULE | INHIBITOR OF | CA1 | | | | |
| SMALL MOLECULE | INHIBITOR OF | CA12 | | | | |
| SMALL MOLECULE | INHIBITOR OF | CA2 | | | | |
| SMALL MOLECULE | INHIBITOR OF | CA4 | | | | |
| SMALL MOLECULE | INHIBITOR OF | CA5A | | | | |
| SMALL MOLECULE | INHIBITOR OF | CA7 | | | | |
| SMALL MOLECULE | INHIBITOR OF | CA9 | | | | |
| SMALL MOLECULE | INHIBITOR OF | CAPN1 | | | | |
| SMALL MOLECULE | INHIBITOR OF | CBX1 | | | | |
| SMALL MOLECULE | INHIBITOR OF | CDK2 | | | | |
| SMALL MOLECULE | INHIBITOR OF | CDK7 | | | | |
| SMALL MOLECULE | INHIBITOR OF | CHEK2 | | | | |
| SMALL MOLECULE | INHIBITOR OF | CLK2 | | | | |
| SMALL MOLECULE | INHIBITOR OF | CLK4 | | | | |
| SMALL MOLECULE | INHIBITOR OF | CREBBP | | | | |
| SMALL MOLECULE | INHIBITOR OF | CSF1R | | | | |
| SMALL MOLECULE | INHIBITOR OF | CTSB | | | | |
| SMALL MOLECULE | INHIBITOR OF | CTSK | | | | |
| SMALL MOLECULE | INHIBITOR OF | CTSL | | | | |
| SMALL MOLECULE | INHIBITOR OF | CTSS | | | | |
| SMALL MOLECULE | INHIBITOR OF | DPP7 | | | | |
| SMALL MOLECULE | INHIBITOR OF | DRD2 | | | | |
| SMALL MOLECULE | INHIBITOR OF | DRD3 | | | | |
| SMALL MOLECULE | INHIBITOR OF | DRD4 | | | | |
| SMALL MOLECULE | INHIBITOR OF | DRD5 | | | | |
| SMALL MOLECULE | INHIBITOR OF | EBP | | | | |
| SMALL MOLECULE | INHIBITOR OF | EGFR | | | | |
| SMALL MOLECULE | INHIBITOR OF | EPHA6 | | | | |
| SMALL MOLECULE | INHIBITOR OF | EPHA8 | | | | |
| SMALL MOLECULE | INHIBITOR OF | ERBB2 | | | | |
| SMALL MOLECULE | INHIBITOR OF | EYA2 | | | | |
| SMALL MOLECULE | INHIBITOR OF | FAAH | | | | |
| SMALL MOLECULE | INHIBITOR OF | FEN1 | | | | |
| SMALL MOLECULE | INHIBITOR OF | FLT1 | | | | |
| SMALL MOLECULE | INHIBITOR OF | FLT3 | | | | |
| SMALL MOLECULE | INHIBITOR OF | FLT4 | | | | |
| SMALL MOLECULE | INHIBITOR OF | FYN | | | | |
| SMALL MOLECULE | INHIBITOR OF | GBA | | | | |
| SMALL MOLECULE | INHIBITOR OF | GFER | | | | |
| SMALL MOLECULE | INHIBITOR OF | HDAC1 | | | | |
| SMALL MOLECULE | INHIBITOR OF | HDAC10 | | | | |
| SMALL MOLECULE | INHIBITOR OF | HDAC11 | | | | |
| SMALL MOLECULE | INHIBITOR OF | HDAC2 | | | | |
| SMALL MOLECULE | INHIBITOR OF | HDAC3 | | | | |
| SMALL MOLECULE | INHIBITOR OF | HDAC4 | | | | |
| SMALL MOLECULE | INHIBITOR OF | HDAC5 | | | | |
| SMALL MOLECULE | INHIBITOR OF | HDAC6 | | | | |
| SMALL MOLECULE | INHIBITOR OF | HDAC7 | | | | |
| SMALL MOLECULE | INHIBITOR OF | HDAC8 | | | | |
| SMALL MOLECULE | INHIBITOR OF | HDAC9 | | | | |
| SMALL MOLECULE | INHIBITOR OF | HRH1 | | | | |
| SMALL MOLECULE | INHIBITOR OF | HSP90AA1 | | | | |
| SMALL MOLECULE | INHIBITOR OF | HSP90AB1 | | | | |
| SMALL MOLECULE | INHIBITOR OF | HSPA5 | | | | |
| SMALL MOLECULE | INHIBITOR OF | HTR2A | | | | |
| SMALL MOLECULE | INHIBITOR OF | HTR2B | | | | |
| SMALL MOLECULE | INHIBITOR OF | HTR2C | | | | |
| SMALL MOLECULE | INHIBITOR OF | HTR5B | | | | |
| SMALL MOLECULE | INHIBITOR OF | HTR6 | | | | |
| SMALL MOLECULE | INHIBITOR OF | HTR7 | | | | |
| SMALL MOLECULE | INHIBITOR OF | IGF1R | | | | |
| SMALL MOLECULE | INHIBITOR OF | IKBKB | | | | |
| SMALL MOLECULE | INHIBITOR OF | IKBKE | | | | |
| SMALL MOLECULE | INHIBITOR OF | INSR | | | | |
| SMALL MOLECULE | INHIBITOR OF | KCNH2 | | | | |
| SMALL MOLECULE | INHIBITOR OF | KDR | | | | |
| SMALL MOLECULE | INHIBITOR OF | KIT | | | | |
| SMALL MOLECULE | INHIBITOR OF | LCK | | | | |
| SMALL MOLECULE | INHIBITOR OF | LTA4H | | | | |
| SMALL MOLECULE | INHIBITOR OF | LYN | | | | |
| SMALL MOLECULE | INHIBITOR OF | MAP4K5 | | | | |
| SMALL MOLECULE | INHIBITOR OF | MAPK12 | | | | |
| SMALL MOLECULE | INHIBITOR OF | MAPK14 | | | | |
| SMALL MOLECULE | INHIBITOR OF | MAPKAPK2 | | | | |
| SMALL MOLECULE | INHIBITOR OF | MMP1 | | | | |
| SMALL MOLECULE | INHIBITOR OF | MMP13 | | | | |
| SMALL MOLECULE | INHIBITOR OF | MMP14 | | | | |
| SMALL MOLECULE | INHIBITOR OF | MMP3 | | | | |
| SMALL MOLECULE | INHIBITOR OF | MMP8 | | | | |
| SMALL MOLECULE | INHIBITOR OF | MTOR | | | | |
| SMALL MOLECULE | INHIBITOR OF | NFKB1 | | | | |
| SMALL MOLECULE | INHIBITOR OF | NPC1 | | | | |
| SMALL MOLECULE | INHIBITOR OF | PABPC1 | | | | |
| SMALL MOLECULE | INHIBITOR OF | PDGFRA | | | | |
| SMALL MOLECULE | INHIBITOR OF | PDGFRB | | | | |
| SMALL MOLECULE | INHIBITOR OF | PGGT1B | | | | |
| SMALL MOLECULE | INHIBITOR OF | PIK3C2A | | | | |
| SMALL MOLECULE | INHIBITOR OF | PIK3C2B | | | | |
| SMALL MOLECULE | INHIBITOR OF | PIK3CA | | | | |
| SMALL MOLECULE | INHIBITOR OF | PIK3CB | | | | |
| SMALL MOLECULE | INHIBITOR OF | PIK3CD | | | | |
| SMALL MOLECULE | INHIBITOR OF | PIK3CG | | | | |
| SMALL MOLECULE | INHIBITOR OF | PIK3R1 | | | | |
| SMALL MOLECULE | INHIBITOR OF | PLK1 | | | | |
| SMALL MOLECULE | INHIBITOR OF | PLK3 | | | | |
| SMALL MOLECULE | INHIBITOR OF | PPARA | | | | |
| SMALL MOLECULE | INHIBITOR OF | PPARD | | | | |
| SMALL MOLECULE | INHIBITOR OF | PPARG | | | | |
| SMALL MOLECULE | INHIBITOR OF | PRKAA1 | | | | |
| SMALL MOLECULE | INHIBITOR OF | PRKAG3 | | | | |
| SMALL MOLECULE | INHIBITOR OF | PRKDC | | | | |
| SMALL MOLECULE | INHIBITOR OF | PSEN2 | | | | |
| SMALL MOLECULE | INHIBITOR OF | RECQL | | | | |
| SMALL MOLECULE | INHIBITOR OF | SENP6 | | | | |
| SMALL MOLECULE | INHIBITOR OF | SENP7 | | | | |
| SMALL MOLECULE | INHIBITOR OF | SENP8 | | | | |
| SMALL MOLECULE | INHIBITOR OF | SLC6A2 | | | | |
| SMALL MOLECULE | INHIBITOR OF | SLC6A3 | | | | |
| SMALL MOLECULE | INHIBITOR OF | SLC6A4 | | | | |
| SMALL MOLECULE | INHIBITOR OF | SMN2 | | | | |
| SMALL MOLECULE | INHIBITOR OF | SRC | | | | |
| SMALL MOLECULE | INHIBITOR OF | TARDBP | | | | |
| SMALL MOLECULE | INHIBITOR OF | TBXAS1 | | | | |
| SMALL MOLECULE | INHIBITOR OF | TCF7L2 | | | | |
| SMALL MOLECULE | INHIBITOR OF | TFPI | | | | |
| SMALL MOLECULE | INHIBITOR OF | TUBB6 | | | | |
| SMALL MOLECULE | INHIBITOR OF | USP2 | | | | |
| SMALL MOLECULE | INHIBITOR OF | WRN | | | | |
| RNAi | INHIBITOR OF | ABCB1 | | | | |
| RNAi | INHIBITOR OF | ABCB11 | | | | |
| RNAi | INHIBITOR OF | ABCG2 | | | | |
| RNAi | INHIBITOR OF | ADAM17 | | | | |
| RNAi | INHIBITOR OF | ADRA1A | | | | |
| RNAi | INHIBITOR OF | ADRA1B | | | | |
| RNAi | INHIBITOR OF | ADRA1D | | | | |
| RNAi | INHIBITOR OF | ADRA2A | | | | |
| RNAi | INHIBITOR OF | ADRA2B | | | | |
| RNAi | INHIBITOR OF | ADRA2C | | | | |
| RNAi | INHIBITOR OF | ATXN2 | | | | |
| RNAi | INHIBITOR OF | BACE1 | | | | |
| RNAi | INHIBITOR OF | BACE2 | | | | |
| RNAi | INHIBITOR OF | BLM | | | | |
| RNAi | INHIBITOR OF | BRD2 | | | | |
| RNAi | INHIBITOR OF | BRD3 | | | | |
| RNAi | INHIBITOR OF | BRD4 | | | | |
| RNAI | INHIBITOR OF | CA1 | | | | |
| RNAi | INHIBITOR OF | CA12 | | | | |
| RNAi | INHIBITOR OF | CA2 | | | | |
| RNAi | INHIBITOR OF | CA4 | | | | |
| RNAi | INHIBITOR OF | CA5A | | | | |
| RNAi | INHIBITOR OF | CA7 | | | | |
| RNAi | INHIBITOR OF | CA9 | | | | |
| RNAi | INHIBITOR OF | CAPN1 | | | | |
| RNAi | INHIBITOR OF | CBX1 | | | | |
| RNAi | INHIBITOR OF | CDK2 | | | | |
| RNAi | INHIBITOR OF | CDK7 | | | | |
| RNAi | INHIBITOR OF | CHEK2 | | | | |
| RNAi | INHIBITOR OF | CLK2 | | | | |
| RNAi | INHIBITOR OF | CLK4 | | | | |
| RNAi | INHIBITOR OF | CREBBP | | | | |
| RNAi | INHIBITOR OF | CSF1R | | | | |
| RNAi | INHIBITOR OF | CTSB | | | | |
| RNAi | INHIBITOR OF | CTSK | | | | |
| RNAi | INHIBITOR OF | CTSL | | | | |
| RNAi | INHIBITOR OF | CTSS | | | | |
| RNAI | INHIBITOR OF | DPP7 | | | | |
| RNAi | INHIBITOR OF | DRD2 | | | | |
| RNAi | INHIBITOR OF | DRD3 | | | | |
| RNAi | INHIBITOR OF | DRD4 | | | | |
| RNAi | INHIBITOR OF | DRD5 | | | | |
| RNAi | INHIBITOR OF | EBP | | | | |
| RNAi | INHIBITOR OF | EGFR | | | | |
| RNAi | INHIBITOR OF | EPHA6 | | | | |
| RNAi | INHIBITOR OF | EPHA8 | | | | |
| RNAi | INHIBITOR OF | ERBB2 | | | | |
| RNAi | INHIBITOR OF | EYA2 | | | | |
| RNAi | INHIBITOR OF | FAAH | | | | |
| RNAi | INHIBITOR OF | FEN1 | | | | |
| RNAi | INHIBITOR OF | FLT1 | | | | |
| RNAi | INHIBITOR OF | FLT3 | | | | |
| RNAi | INHIBITOR OF | FLT4 | | | | |
| RNAi | INHIBITOR OF | FYN | | | | |
| RNAi | INHIBITOR OF | GBA | | | | |
| RNAi | INHIBITOR OF | GFER | | | | |
| RNAi | INHIBITOR OF | HDAC1 | | | | |
| RNAi | INHIBITOR OF | HDAC10 | | | | |
| RNAi | INHIBITOR OF | HDAC11 | | | | |
| RNAi | INHIBITOR OF | HDAC2 | | | | |
| RNAi | INHIBITOR OF | HDAC3 | | | | |
| RNAi | INHIBITOR OF | HDAC4 | | | | |
| RNAi | INHIBITOR OF | HDAC5 | | | | |
| RNAi | INHIBITOR OF | HDAC6 | | | | |
| RNAi | INHIBITOR OF | HDAC7 | | | | |
| RNAi | INHIBITOR OF | HDAC8 | | | | |
| RNAi | INHIBITOR OF | HDAC9 | | | | |
| RNAi | INHIBITOR OF | HRH1 | | | | |
| RNAi | INHIBITOR OF | HSP90AA1 | | | | |
| RNAi | INHIBITOR OF | HSP90AB1 | | | | |
| RNAi | INHIBITOR OF | HSPA5 | | | | |
| RNAi | INHIBITOR OF | HTR2A | | | | |
| RNAi | INHIBITOR OF | HTR2B | | | | |
| RNAi | INHIBITOR OF | HTR2C | | | | |
| RNAi | INHIBITOR OF | HTR5B | | | | |
| RNAi | INHIBITOR OF | HTR6 | | | | |
| RNAi | INHIBITOR OF | HTR7 | | | | |
| RNAi | INHIBITOR OF | IGF1R | | | | |
| RNAi | INHIBITOR OF | IKBKB | | | | |
| RNAi | INHIBITOR OF | IKBKE | | | | |
| RNAi | INHIBITOR OF | INSR | | | | |
| RNAi | INHIBITOR OF | KCNH2 | | | | |
| RNAi | INHIBITOR OF | KDR | | | | |
| RNAi | INHIBITOR OF | KIT | | | | |
| RNAi | INHIBITOR OF | LCK | | | | |
| RNAi | INHIBITOR OF | LTA4H | | | | |
| RNAi | INHIBITOR OF | LYN | | | | |
| RNAi | INHIBITOR OF | MAP4K5 | | | | |
| RNAi | INHIBITOR OF | MAPK12 | | | | |
| RNAi | INHIBITOR OF | MAPK14 | | | | |
| RNAi | INHIBITOR OF | MAPKAPK2 | | | | |
| RNAi | INHIBITOR OF | MMP1 | | | | |
| RNAi | INHIBITOR OF | MMP13 | | | | |
| RNAi | INHIBITOR OF | MMP14 | | | | |
| RNAi | INHIBITOR OF | MMP3 | | | | |
| RNAi | INHIBITOR OF | MMP8 | | | | |
| RNAi | INHIBITOR OF | MTOR | | | | |
| RNAi | INHIBITOR OF | NFKB1 | | | | |
| RNAi | INHIBITOR OF | NPC1 | | | | |
| RNAi | INHIBITOR OF | PABPC1 | | | | |
| RNAi | INHIBITOR OF | PDGFRA | | | | |
| RNAi | INHIBITOR OF | PDGFRB | | | | |
| RNAi | INHIBITOR OF | PGGT1B | | | | |
| RNAi | INHIBITOR OF | PIK3C2A | | | | |
| RNAi | INHIBITOR OF | PIK3C2B | | | | |
| RNAi | INHIBITOR OF | PIK3CA | | | | |
| RNAi | INHIBITOR OF | PIK3CB | | | | |
| RNAi | INHIBITOR OF | PIK3CD | | | | |
| RNAi | INHIBITOR OF | PIK3CG | | | | |
| RNAi | INHIBITOR OF | PIK3R1 | | | | |
| RNAi | INHIBITOR OF | PLK1 | | | | |
| RNAi | INHIBITOR OF | PLK3 | | | | |
| RNAi | INHIBITOR OF | PPARA | | | | |
| RNAi | INHIBITOR OF | PPARD | | | | |
| RNAi | INHIBITOR OF | PPARG | | | | |
| RNAi | INHIBITOR OF | PRKAA1 | | | | |
| RNAi | INHIBITOR OF | PRKAG3 | | | | |
| RNAi | INHIBITOR OF | PRKDC | | | | |
| RNAi | INHIBITOR OF | PSEN2 | | | | |
| RNAi | INHIBITOR OF | RECQL | | | | |
| RNAi | INHIBITOR OF | SENP6 | | | | |
| RNAi | INHIBITOR OF | SENP7 | | | | |
| RNAi | INHIBITOR OF | SENP8 | | | | |
| RNAi | INHIBITOR OF | SLC6A2 | | | | |
| RNAi | INHIBITOR OF | SLC6A3 | | | | |
| RNAi | INHIBITOR OF | SLC6A4 | | | | |
| RNAi | INHIBITOR OF | SMN2 | | | | |
| RNAi | INHIBITOR OF | SRC | | | | |
| RNAi | INHIBITOR OF | TARDBP | | | | |
| RNAi | INHIBITOR OF | TBXAS1 | | | | |
| RNAi | INHIBITOR OF | TCF7L2 | | | | |
| RNAi | INHIBITOR OF | TFPI | | | | |
| RNAi | INHIBITOR OF | TUBB6 | | | | |
| RNAi | INHIBITOR OF | USP2 | | | | |
| RNAi | INHIBITOR OF | WRN | | | | |
| | INHIBITOR OF | ABCB1 | | | | |
| | INHIBITOR OF | ABCB11 | | | | |
| | INHIBITOR OF | ABCG2 | | | | |
| | INHIBITOR OF | ADAM17 | | | | |
| | INHIBITOR OF | ADRA1A | | | | |
| | INHIBITOR OF | ADRA1B | | | | |
| | INHIBITOR OF | ADRA1D | | | | |
| | INHIBITOR OF | ADRA2A | | | | |
| | INHIBITOR OF | ADRA2B | | | | |
| | INHIBITOR OF | ADRA2C | | | | |
| | INHIBITOR OF | ATXN2 | | | | |
| | INHIBITOR OF | BACE1 | | | | |
| | INHIBITOR OF | BACE2 | | | | |
| | INHIBITOR OF | BLM | | | | |
| | INHIBITOR OF | BRD2 | | | | |
| | INHIBITOR OF | BRD3 | | | | |
| | INHIBITOR OF | BRD4 | | | | |
| | INHIBITOR OF | CA1 | | | | |
| | INHIBITOR OF | CA12 | | | | |
| | INHIBITOR OF | CA2 | | | | |
| | INHIBITOR OF | CA4 | | | | |
| | INHIBITOR OF | CA5A | | | | |
| | INHIBITOR OF | CA7 | | | | |
| | INHIBITOR OF | CA9 | | | | |
| | INHIBITOR OF | CAPN1 | | | | |
| | INHIBITOR OF | CBX1 | | | | |
| | INHIBITOR OF | CDK2 | | | | |
| | INHIBITOR OF | CDK7 | | | | |
| | INHIBITOR OF | CHEK2 | | | | |
| | INHIBITOR OF | CLK2 | | | | |
| | INHIBITOR OF | CLK4 | | | | |
| | INHIBITOR OF | CREBBP | | | | |
| | INHIBITOR OF | CSF1R | | | | |
| | INHIBITOR OF | CTSB | | | | |
| | INHIBITOR OF | CTSK | | | | |
| | INHIBITOR OF | CTSL | | | | |
| | INHIBITOR OF | CTSS | | | | |
| | INHIBITOR OF | DPP7 | | | | |
| | INHIBITOR OF | DRD2 | | | | |
| | INHIBITOR OF | DRD3 | | | | |
| | INHIBITOR OF | DRD4 | | | | |
| | INHIBITOR OF | DRD5 | | | | |
| | INHIBITOR OF | EBP | | | | |
| | INHIBITOR OF | EGFR | | | | |
| | INHIBITOR OF | EPHA6 | | | | |
| | INHIBITOR OF | EPHA8 | | | | |
| | INHIBITOR OF | ERBB2 | | | | |
| | INHIBITOR OF | EYA2 | | | | |
| | INHIBITOR OF | FAAH | | | | |
| | INHIBITOR OF | FEN1 | | | | |
| | INHIBITOR OF | FLT1 | | | | |
| | INHIBITOR OF | FLT3 | | | | |
| | INHIBITOR OF | FLT4 | | | | |
| | INHIBITOR OF | FYN | | | | |
| | INHIBITOR OF | GBA | | | | |
| | INHIBITOR OF | GFER | | | | |
| | INHIBITOR OF | HDAC1 | | | | |
| | INHIBITOR OF | HDAC10 | | | | |
| | INHIBITOR OF | HDAC11 | | | | |
| | INHIBITOR OF | HDAC2 | | | | |
| | INHIBITOR OF | HDAC3 | | | | |
| | INHIBITOR OF | HDAC4 | | | | |
| | INHIBITOR OF | HDAC5 | | | | |
| | INHIBITOR OF | HDAC6 | | | | |
| | INHIBITOR OF | HDAC7 | | | | |
| | INHIBITOR OF | HDAC8 | | | | |
| | INHIBITOR OF | HDAC9 | | | | |
| | INHIBITOR OF | HRH1 | | | | |
| | INHIBITOR OF | HSP90AA1 | | | | |
| | INHIBITOR OF | HSP90AB1 | | | | |
| | INHIBITOR OF | HSPA5 | | | | |
| | INHIBITOR OF | HTR2A | | | | |
| | INHIBITOR OF | HTR2B | | | | |
| | INHIBITOR OF | HTR2C | | | | |
| | INHIBITOR OF | HTR5B | | | | |
| | INHIBITOR OF | HTR6 | | | | |
| | INHIBITOR OF | HTR7 | | | | |
| | INHIBITOR OF | IGF1R | | | | |
| | INHIBITOR OF | IKBKB | | | | |
| | INHIBITOR OF | IKBKE | | | | |
| | INHIBITOR OF | INSR | | | | |
| | INHIBITOR OF | KCNH2 | | | | |
| | INHIBITOR OF | KDR | | | | |
| | INHIBITOR OF | KIT | | | | |
| | INHIBITOR OF | LCK | | | | |
| | INHIBITOR OF | LTA4H | | | | |
| | INHIBITOR OF | LYN | | | | |
| | INHIBITOR OF | MAP4K5 | | | | |
| | INHIBITOR OF | MAPK12 | | | | |
| | INHIBITOR OF | MAPK14 | | | | |
| | INHIBITOR OF | MAPKAPK2 | | | | |
| | INHIBITOR OF | MMP1 | | | | |
| | INHIBITOR OF | MMP13 | | | | |
| | INHIBITOR OF | MMP14 | | | | |
| | INHIBITOR OF | MMP3 | | | | |
| | INHIBITOR OF | MMP8 | | | | |
| | INHIBITOR OF | MTOR | | | | |
| | INHIBITOR OF | NFKB1 | | | | |
| | INHIBITOR OF | NPC1 | | | | |
| | INHIBITOR OF | PABPC1 | | | | |
| | INHIBITOR OF | PDGFRA | | | | |
| | INHIBITOR OF | PDGFRB | | | | |
| | INHIBITOR OF | PGGT1B | | | | |
| | INHIBITOR OF | PIK3C2A | | | | |
| | INHIBITOR OF | PIK3C2B | | | | |
| | INHIBITOR OF | PIK3CA | | | | |
| | INHIBITOR OF | PIK3CB | | | | |
| | INHIBITOR OF | PIK3CD | | | | |
| | INHIBITOR OF | PIK3CG | | | | |
| | INHIBITOR OF | PIK3R1 | | | | |
| | INHIBITOR OF | PLK1 | | | | |
| | INHIBITOR OF | PLK3 | | | | |
| | INHIBITOR OF | PPARA | | | | |
| | INHIBITOR OF | PPARD | | | | |
| | INHIBITOR OF | PPARG | | | | |
| | INHIBITOR OF | PRKAA1 | | | | |
| | INHIBITOR OF | PRKAG3 | | | | |
| | INHIBITOR OF | PRKDC | | | | |
| | INHIBITOR OF | PSEN2 | | | | |
| | INHIBITOR OF | RECQL | | | | |
| | INHIBITOR OF | SENP6 | | | | |
| | INHIBITOR OF | SENP7 | | | | |
| | INHIBITOR OF | SENP8 | | | | |
| | INHIBITOR OF | SLC6A2 | | | | |
| | INHIBITOR OF | SLC6A3 | | | | |
| | INHIBITOR OF | SLC6A4 | | | | |
| | INHIBITOR OF | SMN2 | | | | |
| | INHIBITOR OF | SRC | | | | |
| | INHIBITOR OF | TARDBP | | | | |
| | INHIBITOR OF | TBXAS1 | | | | |
| | INHIBITOR OF | TCF7L2 | | | | |
| | INHIBITOR OF | TFPI | | | | |
| | INHIBITOR OF | TUBB6 | | | | |
| | INHIBITOR OF | USP2 | | | | |
| | INHIBITOR OF | WRN | | | | |

ADDITIONAL FIELDS TO TABLE 1ab pharmaceutically acceptable salt of any one of the compounds of this table 1ab, solvate of any one of the compounds of this table 1ab, hydrate of any one of the compounds of this table 1ab; tautomer of any one of the compounds of this table 1ab, geometric isomer of any one of the compounds of this table 1ab, optical isomer of any one of the compounds of this table 1ab stereoisomer of any one of the compounds of this table 1ab, structural analog of any one of the compounds of this table 1ab, functional analog of any one of the compounds of this table 1ab, derivative of any one of the compounds of this table 1ab, prodrug of any one of the compounds of this table 1ab, human senolytic cell modified by any one of the compounds of this table 1ab, human cell modified by any one of the compounds of this table 1ab.

TABLE 1c

| | chembl_ | | full_ | | | |
|---|---|---|---|---|---|---|
| inchi_keys | id | pref_name | molformula | synonyms | trade_names | Target |
| HIYM | CHEMBL | | C22H16O4 | | | ABCB1 |
| WHBM | 165418 | | | | | |
| OOSAEF- | | | | | | |
| UHFFF | | | | | | |
| AOYSA-N | | | | | | |
| PMATZ | CHEMBL | CYCLOSPORINE | C62H111N11O12 | Ciclosporin; Cyclosporine; | Capimune; Capsorin; | ABCB1 |
| TZNYR | 160 | | | Cyclosporin A; 27- | Cequa; Cyclosporine; | |
| CHOR- | | | | 400; ANTIBIOTIC S- | Deximune; Gengraf; Ikervis; | |
| CGLBZJ | | | | 7481F1; NSC-290193; OL- | Neoral; Optimmune; | |
| NRSA- | | | | 27-400; SANG-35; SDZ- | Restasis; Restasis | |
| N | | | | OXL-400 | multidose; Sandimmun; | |
| | | | | | Sandimmune; | |
| | | | | | Sangcya; Vanquoral | |
| WMB | CHEMBL | EPIGALOCATECHIN | C22H18O11 | (-)-Epigallocatechin | | ABCB1 |
| WREP | 297453 | GALLATE | | Gallate; Epigalocatechin | | |
| UVVBIL | | | | gallate | | |
| R- | | | | | | |
| WIYYLY | | | | | | |
| MNSA- | | | | | | |
| N | | | | | | |
| SPBDX | CHEMBL | IVERMECTIN | C95H146O28 | Ivermectin; Ivermectin | Cardomec; Eqvalan; | ABCB1 |
| SGPUH | 1200633 | | | Component B1A; CD- | Heartguard; Ivermectin; | |
| CETR- | | | | 5024; CD5024; L | Ivomec; Mectizan; Sklice; | |
| JFUDT | | | | 640471; L-640471; MK | Soolantra; Stromectol | |
| MANS | | | | 933; MK-933 | | |
| A-N | | | | | | |
| SGTNS | CHEMBL | VERAPAMIL | C27H38N2O4 | Verapamil; Calan; Covera- | | ABCB1 |
| NPWRI | 6966 | | | HS; Iproveratril; | | |
| OYBX- | | | | Isoptin; Tarka; | | |
| UHFFF | | | | Verelan; CP-16533- | | |
| AOYSA-N | | | | 1; CP-165331; D- | | |
| | | | | 365; NSC-272306NA | | |
| FUZM | CHEMBL | HERNANDEZINE | C39H44N2O7 | Hernandezine | | ABCB1 |
| QNZAC | 504936 | | | | | |
| IFDBL- | | | | | | |
| KYJUH | | | | | | |
| HDHSA-N | | | | | | |
| QEVHR | CHEMBL | RESERPINE | C33H4ON2O9 | Reserpine; ENT- | Hiserpia; Rau-Sed; | ABCB1 |
| UUCFG | 772 | | | 50146; NSC- | Reserpine; Sandril; Serpalan; | |
| RFIF- | | | | 237659; NSC-59272 | Serpanray; Serpasil; | |
| MDEJG | | | | | Serpate; Serpivite | |
| ZGSSA- | | | | | | |
| N | | | | | | |
| JXLYSJ | CHEMBL | VINBLASTINE | C46H58N4O9 | Vinblastine; Velban; [3H]- | | ABCB1 |
| RDGCG | 159 | | | Vinblastine; NSC-47842 | | |
| ARV- | | | | | | |
| CFWM | | | | | | |
| RBGOS | | | | | | |
| A-N | | | | | | |
| HRRBJ | CHEMBL | NAFTOPIDIL | C24H28N2O3 | Naftopidil | | ADRA1A |
| VNMS | 142635 | | | | | |
| RJFHQ- | | | | | | |
| UHFFF | | | | | | |
| AOYSA-N | | | | | | |
| QZUDB | CHEMBL | CLOZAPINE | C18H19ClN4 | Clozapine; HF- | Clozapine; Clozaril; | ADRA1A |
| NBUXV | 42 | | | 1854; Clozapine | Fazaclo; Fazaclo Odt; | |
| UHMW- | | | | resolution mixture | Versacloz; Zaponex | |
| UHFFF | | | | | | |
| AOYSA-N | | | | | | |
| UCTW | CHEMBL | EPINEPHRINE | C9H13NO3 | Epinephrine; Adrenaline; | Adren Co; | ADRA1A |
| MZQN | 679 | | | Epinephrine HCl; Levo- | Adrenaclick; Adrenalin; | |
| UQWS | | | | Methylamino- | Ana-Guard; Ana-Kit; | |
| LP- | | | | ethanolcatechol; | Anapen; Anaphylactic | |
| VIFPVB | | | | Twinject; NSC- | Shock Drug Kit; Asma- | |
| QESA-N | | | | 62786 | Vydrin; Asthmahaler; | |
| | | | | | Auvi-Q; Bronkaid | |
| | | | | | Mist; Brovon; | |
| | | | | | Emerade; Epi E Z Pen | |
| | | | | | Jr; Epinephrine; | |
| | | | | | Epipen; Epipen E Z | |
| | | | | | Pen; Epipen Jr; Epipen | |
| | | | | | Jr.; Eppy; Gppe Inh | |
| | | | | | Soln; Jext; Medihaler- | |
| | | | | | epi; Primatene; Primatene | |
| | | | | | Mist; Riddobron; | |
| | | | | | Riddofan; Rybarvin; | |
| | | | | | Simplene; Soln Made To | |
| | | | | | Rybarvin For; | |
| | | | | | Sus-Phrine; Sus- | |
| | | | | | phrine sulfite free; | |
| | | | | | Sus-Phrine | |
| | | | | | Sulfite-Free; | |
| | | | | | Symjepi; Twinject | |
| | | | | | 0.15; Twinject 0.3 | |
| JCTYW | CHEMBL | | C18H17NO2 | (-)-Roemerine | | ADRA1A |
| RARKV | 483825 | | | | | |
| GOBK- | | | | | | |
| CQSZA | | | | | | |
| CIVSA- | | | | | | |
| N | | | | | | ADRA1A |
| BLGXF | CHEMBL | YOHIMBINE | C21H26N2O3 | Yohimbine | | |
| ZZNTV | 15245 | | | | | |
| WLAY- | | | | | | |
| SCYLSF | | | | | | |
| HTSA- | | | | | | |
| N | | | | | | |
| WNMJ | CHEMBL | ALFUZOSIN | C19H27N5O4 | Alfuzosin; Uroxatral | | ADRA1A |
| YKCG | 709 | | | | | |
| WZFFK | | | | | | |
| R- | | | | | | |
| UHFFF | | | | | | |
| AOYSA-N | | | | | | |
| DRHKJ | CHEMBL | TAMSULOSIN | C20H28N2O5S | Tamsulosin; HGP- | | ADRA1A |
| LXJIQT | 836 | | | 0412; HIP-1402; | | |
| DTD- | | | | HIP1402 | | |
| OAHLL | | | | | | |
| OKOSA-N | | | | | | |
| SFLSHL | CHEMBL | NOREPINEPHRINE | C8H11NO3 | Norepinephrine; | | ADRA1A |
| FXELFN | 1437 | | | Levophed; | | |
| JZ- | | | | R-norepinephrine | | |
| QMM | | | | | | |
| MGPO | | | | | | |
| BSA-N | | | | | | |
| JWZZK | CHEMBL | ISOPROTERENOL | C11H17NO3 | Isoprenaline; | | ADRA1B |
| OKVBU | 434 | | | Isoproterenol; | | |
| JMES- | | | | Isoproterenol dl- | | |
| UHFFF | | | | form; A-21; NSC- | | |
| AOYSA-N | | | | 33791; NSC-9975 | | |
| METKI | CHEMBL | ATENOLOL | C14H22N2O3 | Atenolol; Esatenolol; | Antipressan; Atenamin; | ADRA1B |
| MKYRP | 24 | | | C07 AB03; ICI 66082; | Atenix-100; Atenix-25; | |
| QLGS- | | | | ICI-66082 | Atenix-50; | |
| UHFFF | | | | | Atenolol; Ibinolo; | |
| AOYSA-N | | | | | Kentol; Tenor | |
| | | | | | min; Tenormin 25; | |
| | | | | | Tenormin L.S.; Totamol; | |
| | | | | | Unibloc; Vasaten | |
| RUZYU | CHEMBL | DOXAZOSIN | C23H25N5O5 | Doxazosin; Cardura; C02 | | ADRA1B |
| OTYCV | 707 | | | CA04; UK-33274 | | |
| RMRZ- | | | | | | |
| UHFFF | | | | | | |
| AOYSA-N | | | | | | |
| IENZQI | CHEMBL | PRAZOSIN | C19H21N5O4 | Prazosin; CP-12299; CP- | | ADRA1B |
| KPVFG | 2 | | | 122991 | | |
| BNW- | | | | | | |
| UHFFF | | | | | | |
| AOYSA-N | | | | | | |
| PLDUP | CHEMBL | FLUPHENAZINE | C22H26F3N3OS | Fluphenazine; Permitil; | | ADRA1D |
| XSUYLZ | 726 | | | Prolixin; NSC-62323 | | |
| YBN- | | | | | | |
| UHFFF | | | | | | |
| AOYSA-N | | | | | | |
| WZHJK | CHEMBL | METERGOLINE | C25H29N3O2 | Metergoline; FI- | Liserdol | ADRA1D |
| EUHNJ | 19215 | | | 6337; MCE | | |
| HDLS- | | | | | | |
| QTGU | | | | | | |
| NEKAS | | | | | | |
| A-N | | | | | | |
| SPXAC | CHEMBL | DIHYDROERGO | C36H45N5O8S | Dihydroergocristine | | ADRA1D |
| GZWW | 1255837 | CRISTINE | | Mesylate | | |
| VIDGR- | | MESYLATE | | | | |
| SPZWA | | | | | | |
| CKZSA- | | | | | | |
| N | | | | | | |
| URKO | CHEMBL | QUETIAPINE | C21H25N3O2S | Quetiapine | Atrolak XL; Biquelle | ADRA1D |
| MYMA | 716 | | | | XL; Brancico | |
| XPYIN | | | | | XL; Ebesque XL; Mintreleq | |
| W- | | | | | XL; Psyquet XL; Seotiapim | |
| UHFFF | | | | | | |
| AOYSA-N | | | | | | |
| JRWZL | CHEMBL | DOBUTAMINE | C18H23NO3 | Dobutamine; Dobutrex; | XL; Seroquel; | ADRA1D |
| RBJNM | 926 | | | Compound | Seroquel XL; Sondate | |
| ZMFE- | | | | 81929; COMPOUND- | XL; Tenprolide XL; | |
| UHFFF | | | | 81929 | Zaluron XL | |
| AOYSA-N | | | | | | |
| BLGXF | CHEMBL | RAUWOLSCINE | C21H26N2O3 | Rauwolscine | | ADRA1D |
| ZZNTV | 10347 | | | | | |
| WLAY- | | | | | | |
| DIRVCL | | | | | | |
| HFSA- | | | | | | |
| N | | | | | | DRD2 |
| VMWN | CHEMBL | APOMORPHINE | C17H17NO2 | Apomorphine; APL- | | |
| QDUV | 53 | | | 130277; VR-040; VR040 | | |
| QKEIO | | | | | | |
| C- | | | | | | |
| CYBM | | | | | | |
| UJFWS | | | | | | |
| A-N | | | | | | |
| VYFYYT | CHEMBL | DOPAMINE | C8H11NO2 | Dopamine; Carbilev; | | DRD2 |
| LLBUK | 59 | | | Intropin; Parcopa; | | |
| UHU- | | | | Sinemet; NSC-173182 | | |
| UHFFF | | | | | | |
| AOYSA-N | | | | | | |
| CTENF | CHEMBL | DOPAMINE | C8H12ClNO2 | Dopamine HCl; ASL- | Dopamin-Natterman; | DRD2 |
| NNZB | 1557 | HYDROCHLORIDE | | 279; NSC-169105 | Dopamine HCl; | |
| MHDD | | | | | Dopmin; Dynatra; | |
| G- | | | | | Intropin; Sabax Dopamin; | |
| UHFFF | | | | | Selectajet | |
| AOYSA-N | | | | | | |
| UWCV | CHEMBL | PERGOLIDE | C20H30N2O3S2 | Pergolide | Celance; Pergolide | DRD2 |
| GPLTG | 1275 | MESYLATE | | mesilate; Pergolide | Mesylate; Permax | |
| ZWHG | | | | Mesylate; LY-127809 | | |
| S- | | | | | | |
| ZORIO | | | | | | |
| USZSA- | | | | | | |
| N | | | | | | |
| CIRSPT | CHEMBL | | C13H14N2 | | | DRD2 |
| XGPFA | 27811 | | | | | |
| XRE- | | | | | | |
| UHFFF | | | | | | |
| AOYSA-N | | | | | | |
| NOOJL | CHEMBL | | C13H24N4O3 | | | DRD2 |
| ZTTWS | 317488 | | | | | |
| NHOX- | | | | | | |
| UWVG | | | | | | |
| GRQHS | | | | | | |
| A-N | | | | | | |
| CEUOR | CHEMBL | ARIPIPRAZOLE | C23H27C12N3O2 | Aripiprazole; OPC- | Abilify; Abilify | DRD2 |
| ZQYGO | 1112 | | | 14597; OPC-31 | Maintena; Abilify | |
| DEFX- | | | | | maintena kit; Abilify mycite | |
| UHFFF | | | | | kit; Aripiprazole | |
| AOYSA-N | | | | | | |
| XTKDA | CHEMBL | AZAPERONE | C19H22FN3O | Azaperone; R-1929 | Stresnil; Suicalm | DRD2 |
| FGWC | 340211 | | | | | |
| DAMP | | | | | | |
| Y- | | | | | | |
| UHFFF | | | | | | |
| AOYSA-N | | | | | | |
| SMAN | CHEMBL | FLUNARIZINE | 26F2NC26H2 | Flunarizine | | DRD2 |
| XXCAT | 30008 | | | | | |
| UTDDT- | | | | | | |
| QPJJXV | | | | | | |
| BHSA- | | | | | | |
| N | | | | | | |
| DERZB | CHEMBL | CINNARIZINE | C26H28N2 | Cinnarizine; Stugeron; | Marzine R.F.; Mylan Travel | DRD2 |
| LKQOC | 43064 | | | Stunarone; Stutgin; 516 | Sickness; Stugeron; | |
| DDDZ- | | | | MD; 516-MD; | Stugeron Fte | |
| JLHYYA | | | | R-1575; R-516 | | |
| GUSA- | | | | | | |
| N | | | | | | |
| NTJOB | CHEMBL | AMISULPRIDE | C17H27N3O4S | Amisulpride; APD- | Solian; Solian 100; Solian | DRD2 |
| XMM | 243712 | | | 421; APD421; | 200; Solian 400; Solian 50 | |
| WNYJF | | | | DAN-2163 | | |
| B- | | | | | | |
| UHFFF | | | | | | |
| AOYSA-N | | | | | | |
| BGRJT | CHEMBL | SULPIRIDE | C15H23N3O4S | Sulpiride; N05AL01; | Dogmatyl; Dolmatil | DRD3 |
| UBHPO | 26 | | | RD-1403 | | |
| OWDU- | | | | | | |
| UHFFF | | | | | | |
| AOYSA-N | | | | | | |
| VNFPB | CHEMBL | CLOTRIMAZOLE | C22H17ClN2 | Clotrimazole; BAY- | Abtrim; Actavall; Candiden; | DRD3 |
| HJOKIV | 104 | | | 5097; GNF-PF-3499; | Canesten; Canesten AF; | |
| QEB- | | | | NSC-257473 | Canesten Crm | |
| UHFFF | | | | | Combi; Canesten | |
| AOYSA-N | | | | | Internal; Canesten | |
| | | | | | VC; Clotrimazole; | |
| | | | | | Fungederm; | |
| | | | | | Gyne-Lotrimin; | |
| | | | | | Gyne-Lotrimin | |
| | | | | | 3; Gyne-Lotrimin | |
| | | | | | 3 Combination | |
| | | | | | Pack; Gyne-Lotrimin | |
| | | | | | Combination Pack; Gynix; | |
| | | | | | Masnoderm; Mycelex; | |
| | | | | | Mycelex-7; Mycelex-7 | |
| | | | | | Combination Pack; | |
| | | | | | Mycelex-G; Mycil | |
| | | | | | Gold; Privacom; | |
| | | | | | Trimysten; Trivagizole 3 | |
| LNEPO | CHEMBL | HALOPERIDOL | C21H23ClFNO2 | Haloperidol; MCN-JR- | Dolpin; Dozic; Fortunan; | DRD3 |
| XFFQS | 54 | | | 1625; NSC-170973; | Haldol; Haldol | |
| ENCJ- | | | | NSC-615296; R-1625 | Solutab; Haloperidol; | |
| UHFFF | | | | | Kentace; | |
| AOYSA-N | | | | | Keselen; Serenace | |
| IBRKLU | CHEMBL | | C10H13NO2 | | | DRD3 |
| SXDYA | 1195032 | | | | | |
| TLG- | | | | | | |
| LURJT | | | | | | |
| MIESA- | | | | | | |
| N | | | | | | |
| AEQDJ | CHEMBL | TETRAHY- | C21H25NO4 | Tetrahydropalmatine; Te | | DRD5 |
| SLRWY | 487182 | DROPALMATINE | | trahydropalmatine I- | | |
| MAQI- | | | | form; (-)- | | |
| KRWD | | | | Tetrahydropalmatine; L- | | |
| ZBQOS | | | | Tetrahydropalmatine | | |
| A-N | | | | | | |
| AFSDN | CHEMBL | ELLAGIC | C14H6O8 | Ellagic Acid; Benzoaric | | EGFR |
| FLWKV | 6246 | ACID | | Acid; NSC-407286; | | |
| MVRB- | | | | NSC-656272 | | |
| UHFFF | | | | | | |
| AOYSA-N | | | | | | |
| BCFGM | CHEMBL | LAPATINIB | C29H26ClFN4O4S | Lapatinib; GSK- | Tyverb | EGFR |
| OOMA | 554 | | | 572016; GW-2016; GW- | | |
| DDAQ | | | | 572016; GW- | | |
| U- | | | | 572016X; GW572016 | | |
| UHFFF | | | | | | |
| AOYSA-N | | | | | | |
| GTTBE | CHEMBL | ERLOTINIB | C22H24ClN3O4 | Erlotinib HCl; CP-358; | Erlotinib HCl; Tarceva | EGFR |
| UCJPZ | 1079742 | HYDROCHLORIDE | | CP-358774-01; CP- | | |
| QMDZ- | | | | 35877401; OSI-774 | | |
| UHFFF | | | | | | |
| AOYSA-N | | | | | | |
| XGALL | CHEMBL | GEFITINIB | C22H24ClFN4O3 | Gefitinib; Iressa; | Iressa | |
| CVXEZ | 939 | | | ZD-1839 | | |
| PNRQ- | | | | | | |
| UHFFF | | | | | | |
| AOYSA-N | | | | | | |
| XNRVG | CHEMBL | LAPATINIB- | C43H44ClFN4O11S3 | Lapatinib | Tykerb | EGFR |
| THNYC | 1201179 | DITOSYLATE | | Ditosylate; Lapatinib | | |
| NCFF- | | | | tosilate | | |
| UHFFF | | | | hydrate; Lapatinib | | |
| AOYSA-N | | | | ditoluenesulfonate | | |
| | | | | monohydrate; Lapatinib; | | |
| | | | | Lapatinib ditosylate | | |
| | | | | monohydrate; GW- | | |
| | | | | 572016F; GW572016F | | |
| REFJW | CHEMBL | QUERCETIN | C15H10O7 | Quercetin; C.I. Natural | | EGFR |
| TPEDVJ | 50 | | | Red | | |
| JIY- | | | | 1; Meletin; Sophoretin; X | | |
| UHFFF | | | | anthaurine; LDN | | |
| | | | | 0052529; LDN- | | |
| AOYSA-N | | | | 0052529; NSC | | |
| | | | | 57655; NSC 9219; NSC- | | |
| | | | | 57655; NSC-9219 | | |
| AAKJLR | CHEMBL | ERLOTINIB | C22H23N3O4 | Erlotinib; CP- | Tarceva | EPHA6 |
| GGTJK | 553 | | | 358,774; CP-358774; | | |
| AMG- | | | | CP-35877401; OSI-774; | | |
| UHFFF | | | | R-1415; RG-1415; Ro- | | |
| AOYSA-N | | | | 508231 | | |
| MLDQJ | CHEMBL | SORAFENIB | C21H16ClF3N4O3 | Sorafenib; BAY-43-9006 | Nexavar | EPHA6 |
| TXFUG | 1336 | | | | | |
| DVEO- | | | | | | |
| UHFFF | | | | | | |
| AOYSA-N | | | | | | |
| WZUV | CHEMBL | HEMATOXYLIN | C16H14O6 | Hematoxylin | | ERBB2 |
| PPKBW | 477197 | | | | | |
| HMQC | | | | | | |
| E- | | | | | | |
| XJKSG | | | | | | |
| UPXSA- | | | | | | |
| N | | | | | | |
| CWVRJ | CHEMBL | CHLOROGENIC | C16H18O9 | Chlorogenic acid; NSC- | | HDAC10 |
| TMFET | 284616 | ACID | | 407296; NSC-70861 | | |
| XNAD- | | | | | | |
| JUHZA | | | | | | |
| CGLSA- | | | | | | |
| N | | | | | | |
| YTKUW | CHEMBL | BENDAMUSTINE | C16H21Cl2N3O2 | Bendamustine | | HDAC2 |
| DBFDA | 487253 | | | | | |
| SYHO- | | | | | | |
| UHFFF | | | | | | |
| AOYSA-N | | | | | | |
| RZEKV | CHEMBL | CELECOXIB | C17H14F3N3O2S | Celecoxib; DFN- | Celebra; Celebrex; | HDAC6 |
| GVHFL | 118 | | | 15; DFN15; SC-58635 | Celecoxib; | |
| EQIL- | | | | | Onsenal | |
| UHFFF | | | | | | |
| AOYSA-N | | | | | | |
| FUWU | CHEMBL | HINOKITIOL | C10H12O2 | beta-thujaplicin | | HDAC8 |
| EFKEXZ | 48310 | | | | | |
| QKKA- | | | | | | |
| UHFFF | | | | | | |
| AOYSA-N | | | | | | |
| NTYJJO | CHEMBL | HISTAMINE | C5H9N3 | Histamine; NSC-33792 | | HRH1 |
| PFIAH | 90 | | | | | |
| URM- | | | | | | |
| UHFFF | | | | | | |
| AOYSA-N | | | | | | |
| KVWD | CHEMBL | OLANZAPINE | C17H20N4S | Olanzapine; LY- | Arkolamyl; Olanzapine; | HRH1 |
| HTXUZ | 715 | | | 170053; LY170053 | Zalasta; Zyprexa; | |
| HCGIO- | | | | | Zyprexa Zydis | |
| UHFFF | | | | | | |
| AOYSA-N | | | | | | |
| ANTSC | CHEMBL | CHLORDIAZEP | C16H14ClN3O | Chlordiazepoxide; A- | Clopoxide; Librelease; | HRH1 |
| NMPP | 451 | OXIDE | | Poxide; Chlordiazachel; | Libritabs; Librium; | |
| GJYLG- | | | | Clopoxide; | Sarlib; Trakipeal; Zeisin | |
| UHFFF | | | | Librium; Lygen; | | |
| AOYSA-N | | | | Chlordiazepoxide civ | | |
| ZMISO | CHEMBL | AZACYCLONOL | C18H21NO | Azacyclonol; Ataractan; | | HRH1 |
| DWVF | 127508 | | | Calmeran; Frenoton; | | |
| HHWN | | | | Frenquel; gamma- | | |
| R- | | | | Pipradol; Psychosan | | |
| UHFFF | | | | | | |
| AOYSA-N | | | | | | |
| MJJALK | CHEMBL | EBASTINE | C32H39NO2 | Ebastine; LAS W- | Kestine | HRH1 |
| DDGIK | 305660 | | | 090; LAS-W-090; RP- | | |
| VBE- | | | | 64305 | | |
| UHFFF | | | | | | |
| AOYSA-N | | | | | | |
| MBUV | CHEMBL | AZELASTINE | C22H24ClN3O | Azelastine | | HRH1 |
| EWMH | 639 | | | | | |
| ONZEQ | | | | | | |
| D- | | | | | | |
| UHFFF | | | | | | |
| AOYSA-N | | | | | | |
| BAINIU | CHEMBL | OXATOMIDE | C27H30N4O | Oxatomide; R-35443 | Tinset | HRH1 |
| MDFU | 13828 | | | | | |
| RPJM- | | | | | | |
| UHFFF | | | | | | |
| AOYSA-N | | | | | | |
| JCCNY | CHEMBL | LORATADINE | C22H23ClN2O2 | Loratadine; Claritin- | Alavert; Children's Claritin; | HRH1 |
| MKQO | 998 | | | D; SCH-29851 | Claritin; Claritin Hives | |
| SZNPW | | | | | Relief; Claritin Hives Relief | |
| UHFFF | | | | | Reditab; Claritin | |
| AOYSA-N | | | | | Reditabs; Clarityn; Clarityn | |
| | | | | | Rapide; Clarityne; Hay- | |
| | | | | | Rite; Loratadine; Loratadine | |
| | | | | | Redidose; Roletra | |
| HVRLZ | CHEMBL | OLOPATADINE | C21H24ClNO3 | Olopatadine HCl; ALO- | Olopatadine | HRH1 |
| EKDTU | 1719 | HYDROCHLORIDE | | 4943A; KW-4679 | HCl; Pataday; Patanase; | |
| EKQH- | | | | | Patanol; Pazeo | |
| NOILC | | | | | | |
| QHBSA-N | | | | | | |
| JAUOIF | CHEMBL | DESLORATADINE | C19H19ClN2 | Desloratadine; SCH- | Aerius; Clarinex; | HRH1 |
| JMECX | 1172 | | | 34117; Loratadine | Desloratadine; | |
| RGI- | | | | related compound | Midetorin; Neoclarityn | |
| UHFFF | | | | a; Loratadine related | | |
| AOYSA-N | | | | compound a rs | | |
| JJCFRY | CHEMBL | CYPROHEPTADINE | C21H21N | Cyproheptadine | | HRH1 |
| NCJDL | 516 | | | | | |
| XIK- | | | | | | |
| UHFFF | | | | | | |
| AOYSA-N | | | | | | |
| WSPO | CHEMBL | CHLORPROTHIXENE | C18H18ClNS | Chlorprothixene; N- | Taractan; Truxal | HRH1 |
| MRSOL | 908 | | | 714; N-714RO 4- | | |
| SGNFJ- | | | | 0403; NSC-18720; - | | |
| AUWJE | | | | RO-40403; Ro-40403 | | |
| WJLSA- | | | | | | |
| N | | | | | | |
| UXBGZ | CHEMBL | | C15H7FN2O2 | 8-Fluorotryptanthrin | | HSP90AA1 |
| QXHNV | 1098875 | | | | | |
| NPFT- | | | | | | |
| UHFFF | | | | | | |
| AOYSA-N | | | | | | |
| PYOUA | CHEMBL | | C11H14N2 | | | HTR2A |
| IQXJAL | 331241 | | | | | |
| PKW- | | | | | | |
| UHFFF | | | | | | |
| AOYSA-N | | | | | | |
| APJYD | CHEMBL | TRYPTAMINE | C10H12N2 | | | |
| QYYAC | 6640 | | | | | HTR2A |
| XCRM- | | | | | | |
| UHFFF | | | | | | |
| AOYSA-N | | | | | | |
| LDCYZ | CHEMBL | OXITRIPTAN | C11H12N2O3 | Oxitriptan; 5- | Levotonine | HTR2A |
| AJDBX | 350221 | | | hydroxytryptophan I- | | |
| YCGN- | | | | form; 5-hydroxy-I- | | |
| VIFPVB | | | | tryptophan | | |
| QESA- | | | | | | |
| N | | | | | | |
| RUZIU | CHEMBL | GLAUCINE | C21H25NO4 | Glaucine | | HTR2A |
| YOSRD | 228082 | | | | | |
| WYQF- | | | | | | |
| HNNXB | | | | | | |
| MFYSA-N | | | | | | |
| ZKIORV | CHEMBL | | C10H11FN2 | 5-Fluorotryptamine | | HTR2A |
| IXEWI | 275628 | | | | | |
| OGB- | | | | | | |
| UHFFF | | | | | | |
| AOYSA-N | | | | | | |
| JTEJPP | CHEMBL | 5- | C11H14N2O | | | HTR2A |
| KMYBD | 8165 | METHOXY- | | | | |
| EMY- | | TRYPTAMINE | | | | |
| UHFFF | | | | | | |
| AOYSA-N | | | | | | |
| KTUFN | CHEMBL | IMATINIB | C29H31N7O | Imatinib; Gleevec; STI- | | HTR2A |
| OKKBV | 941 | | | 571 | | |
| MGRW- | | | | | | |
| UHFFF | | | | | | |
| AOYSA-N | | | | | | |
| QOYH | CHEMBL | FLUSPIRILENE | C29H31F2N3O | Fluspirilene; Imap; | Imap; Redeptin | HTR2B |
| HIBFX | 46516 | | | Redeptin; | | |
| OOAD | | | | MCN-JR-6218; R-6218 | | |
| H- | | | | | | |
| UHFFF | | | | | | |
| AOYSA-N | | | | | | |
| AQHH | CHEMBL | PROPRANOLOL | C16H21NO2 | Propranolol; Inderal; | | HTR2B |
| HDLHH | 27 | | | Inderide-40/25; Inderide- | | |
| XJYJD- | | | | 80/25; AY-64043- | | |
| UHFFF | | | | | | |
| AOYSA-N | | | | | | |
| YEHClC | CHEMBL | PERGOLIDE | C19H26N2S | Pergolide | | HTR2C |
| AEULNI | 531 | | | | | |
| GD- | | | | | | |
| MZMP | | | | | | |
| ZRCHS | | | | | | |
| A-N | | | | | | |
| LBGZM | CHEMBL | | C13H17ClN2 | | | HTR2C |
| MNZW | 2011564 | | | | | |
| GMAG | | | | | | |
| D- | | | | | | |
| UHFFF | | | | | | |
| AOYSA-N | | | | | | |
| VDHFY | CHEMBL | | C14H18N2 | | | HTR2C |
| TROGA | 170854 | | | | | |
| AQAG- | | | | | | |
| UHFFF | | | | | | |
| AOYSA-N | | | | | | |
| SECBX | CHEMBL | | C13H15ClN2 | | | HTR2C |
| DQTD | 2011570 | | | | | |
| NNDSK- | | | | | | |
| UHFFF | | | | | | |
| AOYSA-N | | | | | | |
| ZSTKHS | CHEMBL | 5-MEO- | C13H18N2O | 5-OME-DMT; [2-(5- | | HTR2C |
| QDNIG | 7257 | DMT | | Methoxy-1H-Indol- | | |
| FLM- | | | | 3-YI)-Ethyl]- | | |
| UHFFF | | | | Dimethyl-Amine | | |
| AOYSA-N | | | | | | |
| QSQQ | CHEMBL | | C11H14N2 | alpha-Methyltryptamine | | HTR2C |
| QURBV | 30713 | | | | | |
| YWZKJ- | | | | | | |
| UHFFF | | | | | | |
| AOYSA-N | | | | | | |
| CWOYL | CHEMBL | | C12H12N2 | | | HTR2C |
| IJQLSN | 295234 | | | | | |
| RRN- | | | | | | |
| UHFFF | | | | | | |
| AOYSA-N | | | | | | |
| QZAYG | CHEMBL | SEROTONIN | C10H12N2O | Serotonin; 5-HT; 5- | | HTR2C |
| JVTTN | 39 | | | Hydroxytryptamine | | |
| CVMB- | | | | | | |
| UHFFF | | | | | | |
| AOYSA-N | | | | | | |
| DYLGF | CHEMBL | LUMEFANTRINE | C30H32Cl3NO | Lumefantrine; CPG- | | KCNH2 |
| OYVTX | 38827 | | | 56695; GNF-PF-1971 | | |
| JFJP- | | | | | | |
| MYYYX | | | | | | |
| RDXSA- | | | | | | |
| N | | | | | | |
| HXGBX | CHEMBL | DARIFENACIN | C28H30N2O2 | Darifenacin; Enablex; | | KCNH2 |
| QDTNZ | 1346 | | | UK-88525 | | |
| RUZDI DTESA- | | | | | | |
| N | | | | | | |
| RAPZE | CHEMBL | RISPERIDONE | C23H27FN4O2 | 64766; RCN- | Perseris kit; Risperdal; l | KCNH2 |
| APATH | 85 | | | Risperidone; LY- | Risperda Consta; | |
| NIPO- | | | | 03004; LY03004; | Risperdal Consta Long | |
| UHFFF | | | | N05AX08; | Acting; Risperdal M; | |
| AOYSA-N | | | | R-64,766; R-64-766; R- | Risperdal M-Tab; Risperdal | |
| | | | | 3028; RCN3028 | Quicklet; Risperidone | |
| DOQPX | CHEMBL | VERAPAMIL | C27H39ClN2O4 | Verapamil HCl; CP- | Berkatens; Calan; Calan | KCNH2 |
| TMNIU | 1280 | HYDROCHLORIDE | | 16533-1; LU-20175; | Sr; Cordilox; Cordilox I.V.; | |
| COSY- | | | | NSC-272366; | Cordilox MR 240; | |
| UHFFF | | | | NSC-657799 | Covera; Covera-Hs; | |
| AOYSA-N | | | | | Ethimil MR 240; Geangin; | |
| | | | | | Half Securon SR; | |
| | | | | | Isoptin; Isoptin | |
| | | | | | Sr; Manidon SR; Ranvera | |
| | | | | | MR; Securon; | |
| | | | | | Securon IV; Securon | |
| | | | | | Mid; Securon SR; | |
| | | | | | Univer; Vera-Til SR; | |
| | | | | | Verapamil HCl; | |
| | | | | | Verapress MR | |
| | | | | | 240; Verelan; | |
| | | | | | Verelan pm; Vertab | |
| | | | | | SR 240; Zolvera | |
| ZDIGN | CHEMBL | BROMPHENIRAMINE | C16H19BrN2 | Brompheniramine; BPN; | | KCNH2 |
| SYAAC | 811 | | | Bromfed; Bromfenex; | | |
| HWNL- | | | | Dimetane; Dimetapp; | | |
| UHFFF | | | | Lodrane | | |
| AOYSA-N | | | | | | |
| OGHN | CHEMBL | CARVEDILOL | C24H26N2O4 | Carvedilol; BM | Carvedilol; Coreg; Coreg | KCNH2 |
| VEJMJS | 723 | | | 14.190; BM-14-190; | CR; Eucardic 12.5; Eucardic | |
| YVRP- | | | | BM-14.190; BM- | 25; Eucardic 3.125; | |
| UHFFF | | | | 14190; C07AG02; DQ- | Eucardic 6.25 | |
| AOYSA-N | | | | 2466; SKF-105517 | | |
| FGXWK | CHEMBL | DOMPERIDONE | C22H24ClN5O2 | Domperidone; R-33812 | Domilium; | KCNH2 |
| SZFVQ | 219916 | | | | Evoxin; Motilium; | |
| USTL- | | | | | Motilium 10; Motilium | |
| UHFFF | | | | | Instants; Vivadone | |
| AOYSA-N | | | | | | |
| WINHZ | CHEMBL | SUNITINIB | C22H27FN4O2 | Sunitinib; Sutent; SU- | Sutent | MAP4K5 |
| LLDWR | 535 | | | 011248; SU- | | |
| ZWRT- | | | | 11248; SU011248 | | |
| ATVHP | | | | | | |
| VEESA- | | | | | | |
| N | | | | | | |
| KKZJGL | CHEMBL | MITOXANTRONE | C22H28N4O6 | Mitoxantrone; | Mitoxantrone HCl; | MTOR |
| LVHKM | 58 | | | Novantrone; | Novantrone | |
| TCM- | | | | NSC-279836 | | |
| UHFFF | | | | | | |
| AOYSA-N | | | | | | |
| YCIMN | CHEMBL | CATECHOL | C6H6O2 | Pyrocatechol | | MTOR |
| LLNPG | 280998 | | | | | |
| FGHC- | | | | | | |
| UHFFF | | | | | | |
| AOYSA-N | | | | | | |
| UHDG | CHEMBL | CYTARABINE | C9H13N3O5 | Cytarabine; Ara- | Alexan; Alexan | MTOR |
| CWIW | 803 | | | Cytidine; Arabinosyl | 100; Cytarabine; Cytosar; | |
| MRVC | | | | Cytosine; NSC-287459; | Cytosar-U; Depocyt; | |
| DJ- | | | | U-19920 | Depocyte; Tarabine Pfs | |
| CCXZU | | | | | | |
| QQUSA-N | | | | | | |
| JUVIOZ | CHEMBL | ROTENONE | C23H22O6 | | | MTOR |
| PCNVV | 429023 | | | | | |
| QFO- | | | | | | |
| HBGV | | | | | | |
| WJBIS | | | | | | |
| A-N | | | | | | |
| QFJCIR | CHEMBL | SIROLIMUS | C51H79NO13 | Sirolimus; Rapamycin; | Rapamune; Sirolimus | MTOR |
| LUMZ | 413 | | | AY-22989; L04AA10; | | |
| QUOT- | | | | NSC-226080; SM-88 | | |
| HPLJO | | | | COMPONENT | | |
| QBZSA- | | | | SIROLIMUS; | | |
| N | | | | WY-090217 | | |
| RCINIC | CHEMBL | PACLITAXEL | C47H51NO14 | Paclitaxel; Taxol; ABI- | Abraxane; Pacitaxel; | MTOR |
| ONZNJ | 428647 | | | 007; BMS 181339- | Paclitaxel; Paxene; | |
| XQF- | | | | 01; BMS-181339-01; | Taxol; Taxol 100 | |
| MZXO | | | | DHP 107; MBT 0206; | | |
| DVADS | | | | NK 105; NSC-125973; | | |
| A-N | | | | QW-8184 | | |
| VSJKW | CHEMBL | CAMPTOTHECIN | C20H16N2O4 | NSC-94600 | | MTOR |
| CGYPA | 65 | | | | | |
| HWDS- | | | | | | |
| FQEVS | | | | | | |
| TJZSA- | | | | | | |
| N | | | | | | |
| LUKBX | CHEMBL | RESVERATROL | C14H12O3 | Resveratrol; (E)- | | PIK3CA |
| SAWLP | 165 | | | resveratrol; 3,5,4′- | | |
| MMSZ- | | | | Trihydroxy-trans- | | |
| OWOJ | | | | stilbene; BIA 6-512; | | |
| BTEDS | | | | BIA-6-512; | | |
| A-N | | | | NSC-327430 | | |
| GKIRPK | CHEMBL | ENCLOMIPHENE | C26H28ClNO | Enclomifene; | | SLC6A2 |
| YJQBW | 954 | | | Cisclomiphene; | | |
| NGO- | | | | Enclomiphene; Isomer | | |
| OCEAC | | | | B; Milophene; Serophene; | | |
| IFDSA- | | | | RMI-16289 | | |
| N | | | | | | |
| BFPYW | CHEMBL | ETHINYLESTRADIOL | C20H24O2 | Ethinylestradiol; Enovid; | Estinyl; Etinestryl; | SLC6A2 |
| IDHMR | 691 | | | Enovid- | Feminone; | |
| ZLRN- | | | | E; Ethinyloestradiol; Ethy | Follicoral; Lynoral | |
| SLHNC | | | | nylestradiol; Ginestrene; | | |
| BLASA- | | | | Norinyl; Ovulen; Ovulen- | | |
| N | | | | 21; Ovulen-28; NSC- | | |
| | | | | 10973; Ethinyl Estradiol | | |
| RGLYK | CHEMBL | DIETHYLSTIL- | C18H20O2 | Diethylstilbestrol; Cyren | Apstil; Diethylstilbestrol; | SLC6A3 |
| WWBQ | 411 | BESTROL | | A; Estrobene; Estrogenine; | Distilbene; Stilbestrol; | |
| GJZGM- | | | | Estromenin; Fonatol; | Stilbetin; Stilboesterol; | |
| | | | | New-Estranol | Tampovagan | |
| ISLYRV | | | | 1; Palestrol; Stilbestrofor | | |
| AYSA- | | | | m; Stilbestrol; Stilboestro | | |
| N | | | | I; Synestrin; Synthestrin; | | |
| | | | | NSC-3070 | | |
| SNPPW | CHEMBL | BUPROPION | C13H18ClNO | Bupropion | | SLC6A3 |
| IUOZR | 894 | | | | | |
| MYNY- | | | | | | |
| UHFFF | | | | | | |
| AOYSA-N | | | | | | |
| PNVNV | CHEMBL | VENLAFAXINE | C17H27NO2 | Venlafaxine; Effexor | Alventa XL; Amphero | SLC6A4 |
| HUZRO | 637 | | | | XL; Bonilux | |
| JLTJ- | | | | | XL; Depefex XL; | |
| | | | | | Efexor; Efexor | |
| UHFFF | | | | | XL; Foraven XL; Mentaven | |
| AOYSA-N | | | | | XL; Politid XL; Ranfaxine | |
| | | | | | XL; Rodomel XL; Sunveniz | |
| | | | | | XL; Tardcaps XL; Tifaxin | |
| | | | | | XL; Tonpular XL; Trixat | |
| | | | | | XL; Vaxalin | |
| | | | | | XL; Venaxx XL; Venlablue | |
| | | | | | XL; Venladex XL; Venlalic | |
| | | | | | XL; Venlaneo XL; | |
| | | | | | Vensir XL; Vexarin | |
| | | | | | XL; Viepax; ViePax XL; | |
| | | | | | Winfex XL | |
| RTHCY | CHEMBL | FLUOXETINE | C17H18F3NO | Fluoxetine; Prozac; | | SLC6A4 |
| VBBDH | 41 | | | Sarafem; | | |
| JXIQ- | | | | Symbyax; NSC- | | |
| UHFFF | | | | 283480 | | |
| AOYSA-N | | | | | | |
| MKXZA | CHEMBL | DEXTRO- | C18H25NO | Dextromethorphan; | | SLC6A4 |
| SYAUG | 52440 | METHORPHAN | | Dimetane-DX | | |
| DDCJ- | | | | | | |
| NJAFH | | | | | | |
| UGGSA-N | | | | | | |
| QYRYF | CHEMBL | VENLAFAXINE | C17H28ClNO2 | Venlafaxine HCl; WY- | Effexor; Effexor Xr; | SLC6A4 |
| NHXAR | 1201066 | HYDROCHLORIDE | | 45030 | Venlafaxine | |
| DNFZ- | | | | | HC | |
| UHFFF | | | | | | |
| AOYSA-N | | | | | | |
| ZNRGQ | CHEMBL | TROPISETRON | C17H20N2O2 | Tropisetron; | Navoban | SLC6A4 |
| MMCG | 56564 | | | ICS-205-930 | | |
| HDTEI- | | | | | | |
| ITGUQ | | | | | | |
| SILSA- | | | | | | |
| N | | | | | | |
| WIHM | CHEMBL | CITALOPRAM | C20H22BrFN2O | Citalopram | Celexa; Cipramil; | SLC6A4 |
| BLDNR | 1200781 | HYDROBROMIDE | | Hydrobromide; LU 10- | Citalopram | |
| MIGD | | | | 171-B; LU-10-171-B | Hydrobromide; Paxoran | |
| W- | | | | | | |
| UHFFF | | | | | | |
| AOYSA-N | | | | | | |
| WSEQX | CHEMBL | CITALOPRAM | C20H21FN2O | Citalopram; Celexa | | SLC6A4 |
| VZVJXJ | 549 | | | | | |
| VFP- | | | | | | |
| UHFFF | | | | | | |
| AOYSA-N | | | | | | |
| IUENQ | CHEMBL | | C14H16N2 | | | SLC6A4 |
| RYBZH | 26655 | | | | | |
| HPBN- | | | | | | |
| UHFFF | | | | | | |
| AOYSA-N | | | | | | |
| GIYXAJ | CHEMBL | FLUOXETINE | C17H19ClF3NO | Fluoxetine HCl; LY- | Felicium; Fluoxetine; | SLC6A4 |
| PCNFJE | 1201082 | HYDROCHLORIDE | | 110140; LY110140 | Fluoxetine | |
| HY- | | | | | HCl; Olena; Oxactin; | |
| UHFFF | | | | | Prozac; Prozac 60; Prozac | |
| AOYSA-N | | | | | Weekly; Prozep; Prozit; | |
| | | | | | Ranflutin; Sarafem; Selfemra | |
| INDBQ | CHEMBL | PRIMAQUINE | C15H21N3O | Primaquine; NSC- | | SLC6A4 |
| LZJXZL | 506 | | | 27296; SN-13272; WR- | | |
| FIT- | | | | 2975 | | |
| UHFFF | | | | | | |
| AOYSA-N | | | | | | |
| ADDITIONAL | | | | | | |
| FIELDS | | | | | | |
| TO | | | | | | |
| TABLE | | | | | | |
| 1C | | | | | | |
| pharma- | | | | | | |
| ceutically | | | | | | |
| acceptable | | | | | | |
| salt | | | | | | |
| of | | | | | | |
| any | | | | | | |
| one | | | | | | |
| of | | | | | | |
| the | | | | | | |
| compounds of | | | | | | |
| this | | | | | | |
| table | | | | | | |
| 1c, | | | | | | |
| solvate of | | | | | | |
| anyone | | | | | | |
| of | | | | | | |
| the | | | | | | |
| compounds | | | | | | |
| of | | | | | | |
| this | | | | | | |
| table | | | | | | |
| 1c, | | | | | | |
| hydrate | | | | | | |
| of | | | | | | |
| anyone | | | | | | |
| of | | | | | | |
| the | | | | | | |
| compounds | | | | | | |
| of | | | | | | |
| this | | | | | | |
| table | | | | | | |
| 1c; | | | | | | |
| tautomer | | | | | | |
| of | | | | | | |
| any | | | | | | |
| one | | | | | | |
| of | | | | | | |
| the | | | | | | |
| compounds | | | | | | |
| of | | | | | | |
| this | | | | | | |
| table | | | | | | |
| 1c, | | | | | | |
| geo | | | | | | |
| metric | | | | | | |
| isomer of | | | | | | |
| anyone | | | | | | |
| of | | | | | | |
| the | | | | | | |
| compounds | | | | | | |
| of | | | | | | |
| this | | | | | | |
| table | | | | | | |
| 1c, | | | | | | |
| optical | | | | | | |
| isomer of | | | | | | |
| any | | | | | | |
| one | | | | | | |
| of | | | | | | |
| the | | | | | | |
| compounds | | | | | | |
| of | | | | | | |
| this | | | | | | |
| table | | | | | | |
| 1c | | | | | | |
| stereoisomer | | | | | | |
| of | | | | | | |
| any | | | | | | |
| one | | | | | | |
| of | | | | | | |
| the | | | | | | |
| compounds | | | | | | |
| of | | | | | | |
| this | | | | | | |
| table | | | | | | |
| 1c, | | | | | | |
| structural | | | | | | |
| analog of | | | | | | |
| any | | | | | | |
| one | | | | | | |
| of | | | | | | |
| the | | | | | | |
| compounds | | | | | | |
| of | | | | | | |
| this | | | | | | |
| table | | | | | | |
| 1c, | | | | | | |
| functional | | | | | | |
| analog of | | | | | | |
| anyone | | | | | | |
| of | | | | | | |
| the | | | | | | |
| compounds | | | | | | |
| of | | | | | | |
| this | | | | | | |
| table | | | | | | |
| 1c, | | | | | | |
| derivative | | | | | | |
| of | | | | | | |
| anyone | | | | | | |
| of | | | | | | |
| the | | | | | | |
| compounds | | | | | | |
| of | | | | | | |
| this | | | | | | |
| table | | | | | | |
| 1c, | | | | | | |
| prodrug | | | | | | |
| of | | | | | | |
| anyone | | | | | | |
| of | | | | | | |
| the | | | | | | |
| compounds | | | | | | |
| of | | | | | | |
| this | | | | | | |
| table | | | | | | |
| 1c, | | | | | | |
| human | | | | | | |
| senolytic | | | | | | |
| cell | | | | | | |
| modified | | | | | | |
| by | | | | | | |
| anyone | | | | | | |
| of | | | | | | |
| the | | | | | | |
| compounds | | | | | | |

of
this
table
1c,
human
cell
modified
by
anyone
of
the
compounds
of
this
table
1c.

Accordingly, the present invention also relates to the following claims

1. The compound selected from the group consisting of all compounds listed in the Table 1 ab, Table 1c and Table 1d or its pharmaceutically acceptable salt or solvate, hydrate; tautomer, geometric, optical and stereoisomer thereof, structural analog, functional analog, derivative, prodrug, or compound, having the similar SAR characteristics, mixture thereof in all ratios or combination thereof in all ratios for the use as anti-aging therapy.

2. The compound selected from the group consisting of all compounds listed in the Table 1 ab, Table 1c and Table 1d, for ameliorating at list one symptom of the disorder selected from the group, consisting of aging, frailty, senescence, aging related disease, aging related condition, senescence related disease.

3. An anti-aging pharmaceutical composition, comprising a compound selected from the group consisting of all compounds listed in the Table 1 ab, Table 1c and Table 1d.

4. A method of providing an anti-aging treatment or of treating or preventing an age-related disease or disorder of a subject comprising reducing, inhibiting, or degrading a protein selected from the group consisting of: CDK7, CHEK2, CAPN1, CTSB, HSP90AB1, HSP90AA1, HSPA5, NFKB1, NPC1, PABPC1, ABCB11, ABCB1, PLK1, PLK3, ADRA2A, ADRA2C, ADRA2B, TUBB6, SENP8, SENP6, SENP7, MMP14, MMP13, MMP8, MMP3, MMP1, MAPKAPK2, CTSL, CTSK, CTSS, ATXN2, TBXAS1, CREBBP, PDGFRA, FLT4, FLT1, KDR, PDGFRB, FLT3, KIT, CSF1R, IKBKB, IKBKE, PGGT1B, FAAH, GFER, MAPK14, MAPK12, PRKAA1, ABCG2, ADAM17, BACE2, BACE1, TUBB6, CA7, CA2, CA1, CA5A, CA12, CA9, CA4, CTSB, FEN1, HDAC1, HDAC2, HDAC3, CBX1, SLC6A4, SLC6A2, SLC6A3, PPARD, PPARG, PPARA, PSEN2, HTR7, ADRA1B, SMN2, FYN, SRC, LCK, LYN, TARDBP, CLK2, CLK4, USP2, GBA, RECQL, BLM, WRN, CDK2, PRKAG3, TFPI, LTA4H, DPP7, BACE2, BACE1, CAPN1, CTSB, IGF1R, INSR, EYA2, HSPA5, NFKB1, TCF7L2, TARDBP, BRD4, ABCB1, ADRA1A, ADRA1B, ADRA1D, BRD2, BRD3, BRD4, DRD2, DRD3, DRD4, DRD5, EBP, EGFR, EPHA6, EPHA8, ERBB2, HDAC1, HDAC10, HDAC11, HDAC2, HDAC3, HDAC4, HDAC5, HDAC6, HDAC7, HDAC8, HDAC9, HRH1, HSP90AA1, HTR2A, HTR2B, HTR2C, HTR5B, HTR6, HTR7, KCNH2, MAP4K5, PIK3C2A, PIK3C2B, PIK3CA, PIK3CB, PIK3CD, PIK3CG, PIK3R1, PRKDC, SLC6A2, SLC6A3, SLC6A4 in the organism of the subject, including but not limited to the compound selected from the group consisting of all compounds listed in the Table 1 ab, Table 1c and Table 1d.

5. The method of any one of preceding claims, wherein the age-related disease or disorder is associated with an alleviated level of a protein selected from the group consisting of CDK7, CHEK2, CAPN1, CTSB, HSP90AB1, HSP90AA1, HSPA5, NFKB1, NPC1, PABPC1, ABCB11, ABCB1, PLK1, PLK3, ADRA2A, ADRA2C, ADRA2B, TUBB6, SENP8, SENP6, SENP7, MMP14, MMP13, MMP8, MMP3, MMP1, MAPKAPK2, CTSL, CTSK, CTSS, ATXN2, TBXAS1, CREBBP, PDGFRA, FLT4, FLT1, KDR, PDGFRB, FLT3, KIT, CSF1R, IKBKB, IKBKE, PGGT1B, FAAH, GFER, MAPK14, MAPK12, PRKAA1, ABCG2, ADAM17, BACE2, BACE1, TUBB6, CA7, CA2, CA1, CA5A, CA12, CA9, CA4, CTSB, FEN1, HDAC1, HDAC2, HDAC3, CBX1, SLC6A4, SLC6A2, SLC6A3, PPARD, PPARG, PPARA, PSEN2, HTR7, ADRA1B, SMN2, FYN, SRC, LCK, LYN, TARDBP, CLK2, CLK4, USP2, GBA, RECQL, BLM, WRN, CDK2, PRKAG3, TFPI, LTA4H, DPP7, BACE2, BACE1, CAPN1, CTSB, IGF1R, INSR, EYA2, HSPA5, NFKB1, TCF7L2, TARDBP, BRD4, ABCB1, ADRA1A, ADRA1B, ADRA1D, BRD2, BRD3, BRD4, DRD2, DRD3, DRD4, DRD5, EBP, EGFR, EPHA6, EPHA8, ERBB2, HDAC1, HDAC10, HDAC11, HDAC2, HDAC3, HDAC4, HDAC5, HDAC6, HDAC7, HDAC8, HDAC9, HRH1, HSP90AA1, HTR2A, HTR2B, HTR2C, HTR5B, HTR6, HTR7, KCNH2, MAP4K5, PIK3C2A, PIK3C2B, PIK3CA, PIK3CB, PIK3CD, PIK3CG, PIK3R1, PRKDC, SLC6A2, SLC6A3, SLC6A4 in an organism of the subject or with the increased activity of such protein.

6. The method of any one of preceding claims, wherein the age-related disease or disorder is selected from the group consisting of frailty, Alzheimer's disease, Parkinson's disease, Huntington's diseases, cardiovascular disease, renal failure, muscle wasting or cachexia, osteopenia or osteoporosis, obesity, insulin resistance or diabetes, diverse adult-onset cancers, atherosclerosis, cardiovascular disease, adult cancer, arthritis, cataracts, osteoporosis, type 2 diabetes, hypertension, age-progressive dementia; amyotrophic lateral sclerosis, stroke, atrophic gastritis, osteoarthritis, NASH, camptocormia, chronic obstructive pulmonary disease, coronary artery disease, dopamine dysregulation syndrome, metabolic syndrome, effort incontinence, Hashimoto's thyroiditis, heart failure, late life depression, immunosenescence, age related decline in immune response to vaccines, age related decline in response to immunotherapy, myocardial infarction, acute coronary syndrome, sarcopenia, sarcopenic obesity, senile osteoporosis, urinary incontinence, stroke, atrophic gastritis, camptocormia, chronic obstructive pulmonary disease, coronary artery disease, dopamine dysregulation syndrome, late life depression, osteoarthritis, chronic fatigue syndrome, senile dementia, mild cognitive impairment due to aging, Creutzfeldt-Jakob disease, stroke, CNS cerebral senility, pre-diabetes, diabetes, peripheral arterial disease, aortic valve disease, stroke, Lewy body disease, progressive subcortical gliosis, progressive supranuclear palsy, thalamic degeneration syndrome, hereditary aphasia, myoclonus epilepsy, macular degeneration, pressure ulcers, delirium, progressive subcortical gliosis, progressive supranuclear palsy, thalamic degeneration syndrome, hereditary aphasia, myoclonus epilepsy, and metabolic disorder.

7. The method of any one of preceding claims, wherein the anti-aging treatment is selected from the group consisting of a treatment leading to prevention, amelioration or lessening at least one effect of aging; prevention, amelioration or lessening at least one symptom of aging, prevention, amelioration or lessening at least one symptom of age related disease or condition, decreasing or delaying an increase in a biological age of the subject; slowing a rate of aging of the subject; prevention, amelioration or lessening the effects of frailty; prevention, amelioration or lessening the effects of at least one of an aging-related disease or conditions; increasing a health span or lifespan of the subject; increasing a stress resistance or resilience of the subject; increasing a rate or other enhancement of recovery after surgery, radiotherapy, disease and/or any other stress; prevention, amelioration or lesion the effects of menopausal syndrome; restoring reproductive function; elimination or lessening the spread of senescent cells; modulation of at least one biomarker of aging into the healthier state; a decrease in a rate of wrinkle development; and decrease in a rate of hair greying.

8. The method of any one of preceding claims, wherein the anti-aging treatment is a treatment leading to changing to a healthier state a parameter selected from the group consisting of a blood parameter, a heart rate, a cognitive function, a bone density, a basal metabolic rate, a systolic blood pressure, a heel bone mineral density (BMD), a heel quantitative ultrasound index (QUI), a heel broadband ultrasound attenuation, a forced expiratory volume in 1-second (FEV1), forced vital capacity (FVC), a peak expiratory flow (PEF), a duration to first press of snap-button in each round, a reaction time, a mean time to correctly identify matches, a right or left hand grip strength, a whole body fat-free mass, a leg fat-free mass, a time for recovery after a stress-inducing event, a resistance to radiation, a morbidity risk, and a mortality risk of the subject.

9. A method of providing an anti-aging treatment or of treating or preventing an age-related disease or disorder of a subject, comprising administering to the subject a pharmaceutical composition comprising an inhibitor of a protein selected from the group consisting of: CDK7, CHEK2, CAPN1, CTSB, HSP90AB1, HSP90AA1, HSPA5, NFKB1, NPC1, PABPC1, ABCB11, ABCB1, PLK1, PLK3, ADRA2A, ADRA2C, ADRA2B, TUBB6, SENP8, SENP6, SENP7, MMP14, MMP13, MMP8, MMP3, MMP1, MAPKAPK2, CTSL, CTSK, CTSS, ATXN2, TBXAS1, CREBBP, PDGFRA, FLT4 FLT1, KDR, PDGFRB, FLT3, KIT, CSF1R, IKBKB, IKBKE, PGGT1B, FAAH, GFER, MAPK14, MAPK12, PRKAA1, ABCG2, ADAM17, BACE2, BACE1, TUBB6, CA7, CA2, CA1, CA5A, CA12, CA9, CA4, CTSB, FEN1, HDAC1, HDAC2, HDAC3, CBX1, SLC6A4, SLC6A2, SLC6A3, PPARD, PPARG, PPARA, PSEN2, HTR7, ADRA1B, SMN2, FYN, SRC, LCK, LYN, TARDBP, CLK2, CLK4, USP2, GBA, RECQL, BLM, WRN, CDK2, PRKAG3, TFPI, LTA4H, DPP7, BACE2, BACE1, CAPN1, CTSB, IGF1R, INSR, EYA2, HSPA5, NFKB1, TCF7L2, TARDBP, BRD4, ABCB1, ADRA1A, ADRA1B, ADRA1D, BRD2, BRD3, BRD4, DRD2, DRD3, DRD4, DRD5, EBP, EGFR, EPHA6, EPHA8, ERBB2, HDAC1, HDAC10, HDAC11, HDAC2, HDAC3, HDAC4, HDAC5, HDAC6, HDAC7, HDAC8, HDAC9, HRH1, HSP90AA1, HTR2A, HTR2B, HTR2C, HTR5B, HTR6, HTR7, KCNH2, MAP4K5, PIK3C2A, PIK3C2B, PIK3CA, PIK3CB, PIK3CD, PIK3CG, PIK3R1, PRKDC, SLC6A2, SLC6A3, SLC6A4; and at least one pharmaceutically acceptable excipient.
